SEQUENCE LISTING (1695 sequences)

SEQ ID NO: 1 - protein, 8 aa, synthetic construct
GYTFTRYV

SEQ ID NO: 2 - protein, 8 aa, synthetic construct
INPNNDGT

SEQ ID NO: 3 - protein, 20 aa, synthetic construct
ARGDLEILPYYDHARNAMDY

SEQ ID NO: 4 - protein, 6 aa, synthetic construct
GNIHNY

SEQ ID NO: 5 - skipped

SEQ ID NO: 6 - protein, 9 aa, synthetic construct
QHFWSSPPT

SEQ ID NO: 7 - protein, 5 aa, synthetic construct
RYVMH

SEQ ID NO: 8 - protein, 17 aa, synthetic construct
YINPNNDGTKYNEKFKG

SEQ ID NO: 9 - protein, 18 aa, synthetic construct
GDLEILPYYDHARNAMDY

SEQ ID NO: 10 - protein, 11 aa, synthetic construct
RASGNIHNYLA

SEQ ID NO: 11 - protein, 7 aa, synthetic construct
NAKTLAD

SEQ ID NO: 12 - protein, 9 aa, synthetic construct
QHFWSSPPT

SEQ ID NO: 13 - protein, 7 aa, synthetic construct
GYTFTRY

SEQ ID NO: 14 - protein, 6 aa, synthetic construct
NPNNDG

SEQ ID NO: 15 - protein, 18 aa, synthetic construct
GDLEILPYYDHARNAMDY

SEQ ID NO: 16 - protein, 11 aa, synthetic construct
RASGNIHNYLA

SEQ ID NO: 17 - protein, 7 aa, synthetic construct
NAKTLAD

SEQ ID NO: 18 - protein, 9 aa, synthetic construct
QHFWSSPPT

SEQ ID NO: 19 - protein, 13 aa, synthetic construct
KASGYTFTRYVMH

SEQ ID NO: 20 - protein, 10 aa, synthetic construct
YINPNNDGTK

SEQ ID NO: 21 - protein, 20 aa, synthetic construct
ARGDLEILPYYDHARNAMDY

SEQ ID NO: 22 - protein, 11 aa, synthetic construct
RASGNIHNYLA

SEQ ID NO: 23 - protein, 8 aa, synthetic construct
YNAKTLAD

SEQ ID NO: 24 - protein, 9 aa, synthetic construct
QHFWSSPPT

SEQ ID NO: 25 - protein, 8 aa, synthetic construct
GYTFTRYV

SEQ ID NO: 26 - protein, 8 aa, synthetic construct
INPNNDGT

SEQ ID NO: 27 - protein, 20 aa, synthetic construct
ARGDLEILPYYDHARNAMDY

SEQ ID NO: 28 - protein, 6 aa, synthetic construct
GNIHNY

SEQ ID NO: 29 - skipped

SEQ ID NO: 30 - protein, 9 aa, synthetic construct
QHFWSSPPT

SEQ ID NO: 31 - protein, 8 aa, synthetic construct
GYTFTRYV

SEQ ID NO: 32 - protein, 8 aa, synthetic construct
INPNNDGT

SEQ ID NO: 33 - protein, 20 aa, synthetic construct
ARGDLEILPYYDHARNAMDY

SEQ ID NO: 34 - protein, 6 aa, synthetic construct
ENIYSY

SEQ ID NO: 35 - skipped

SEQ ID NO: 36 - protein, 9 aa, synthetic construct
QHFWSTPPT

SEQ ID NO: 37 - protein, 5 aa, synthetic construct
RYVMH

SEQ ID NO: 38 - protein, 17 aa, synthetic construct
YINPNNDGTKYNEKFKG

SEQ ID NO: 39 - protein, 18 aa, synthetic construct
GDLEILPYYDHARNAMDY

SEQ ID NO: 40 - protein, 11 aa, synthetic construct
RASENIYSYLA

SEQ ID NO: 41 - protein, 7 aa, synthetic construct
NAKTLAE

SEQ ID NO: 42 - protein, 9 aa, synthetic construct
QHFWSTPPT

SEQ ID NO: 43 - protein, 7 aa, synthetic construct
GYTFTRY

SEQ ID NO: 44 - protein, 6 aa, synthetic construct
NPNNDG

SEQ ID NO: 45 - protein, 18 aa, synthetic construct
GDLEILPYYDHARNAMDY

SEQ ID NO: 46 - protein, 11 aa, synthetic construct
RASENIYSYLA

SEQ ID NO: 47 - protein, 7 aa, synthetic construct
NAKTLAE

SEQ ID NO: 48 - protein, 9 aa, synthetic construct
QHFWSTPPT

SEQ ID NO: 49 - protein, 13 aa, synthetic construct
KASGYTFTRYVMH

SEQ ID NO: 50 - protein, 10 aa, synthetic construct
YINPNNDGTK

SEQ ID NO: 51 - protein, 20 aa, synthetic construct
ARGDLEILPYYDHARNAMDY

SEQ ID NO: 52 - protein, 11 aa, synthetic construct
RASENIYSYLA

SEQ ID NO: 53 - protein, 8 aa, synthetic construct
YNAKTLAE

SEQ ID NO: 54 - protein, 9 aa, synthetic construct
QHFWSTPPT

SEQ ID NO: 55 - protein, 8 aa, synthetic construct
GYTFTRYV

SEQ ID NO: 56 - protein, 8 aa, synthetic construct
INPNNDGT

SEQ ID NO: 57 - protein, 20 aa, synthetic construct
ARGDLEILPYYDHARNAMDY

SEQ ID NO: 58 - protein, 6 aa, synthetic construct
ENIYSY

SEQ ID NO: 59 - skipped

SEQ ID NO: 60 - protein, 9 aa, synthetic construct
QHFWSTPPT

SEQ ID NO: 61 - protein, 8 aa, synthetic construct
GYTFTRYV

SEQ ID NO: 62 - protein, 8 aa, synthetic construct
INPNNDGT

SEQ ID NO: 63 - protein, 20 aa, synthetic construct
ARGDLELLPYYDYVRNAMDY

SEQ ID NO: 64 - protein, 6 aa, synthetic construct
GNIHNY

SEQ ID NO: 65 - skipped

SEQ ID NO: 66 - protein, 9 aa, synthetic construct
QHFWSTPPT

SEQ ID NO: 67 - protein, 5 aa, synthetic construct
RYVMY

SEQ ID NO: 68 - protein, 17 aa, synthetic construct
YINPDNDGTKYNEKFKG

SEQ ID NO: 69 - protein, 18 aa, synthetic construct
GDLELLPYYDYVRNAMDY

SEQ ID NO: 70 - protein, 11 aa, synthetic construct
RASGNIHNYLA

SEQ ID NO: 71 - protein, 7 aa, synthetic construct
NAKTLAD

SEQ ID NO: 72 - protein, 9 aa, synthetic construct
QHFWSTPPT

SEQ ID NO: 73 - protein, 7 aa, synthetic construct
GYTFTRY

SEQ ID NO: 74 - protein, 6 aa, synthetic construct
NPDNDG

SEQ ID NO: 75 - protein, 18 aa, synthetic construct
GDLELLPYYDYVRNAMDY

SEQ ID NO: 76 - protein, 11 aa, synthetic construct
RASGNIHNYLA

SEQ ID NO: 77 - protein, 7 aa, synthetic construct
NAKTLAD

SEQ ID NO: 78 - protein, 9 aa, synthetic construct
QHFWSTPPT

SEQ ID NO: 79 - protein, 13 aa, synthetic construct
KASGYTFTRYVMY

SEQ ID NO: 80 - protein, 10 aa, synthetic construct
YINPDNDGTK

SEQ ID NO: 81 - protein, 20 aa, synthetic construct
ARGDLELLPYYDYVRNAMDY

SEQ ID NO: 82 - protein, 11 aa, synthetic construct
RASGNIHNYLA

SEQ ID NO: 83 - protein, 8 aa, synthetic construct
YNAKTLAD

SEQ ID NO: 84 - protein, 9 aa, synthetic construct
QHFWSTPPT

SEQ ID NO: 85 - protein, 8 aa, synthetic construct
GYTFTRYV

SEQ ID NO: 86 - protein, 8 aa, synthetic construct
INPDNDGT

SEQ ID NO: 87 - protein, 20 aa, synthetic construct
ARGDLELLPYYDYVRNAMDY

SEQ ID NO: 88 - protein, 6 aa, synthetic construct
GNIHNY

SEQ ID NO: 89 - skipped

SEQ ID NO: 90 - protein, 9 aa, synthetic construct
QHFWSTPPT

SEQ ID NO: 91 - protein, 8 aa, synthetic construct
GFNIKDYY

SEQ ID NO: 92 - protein, 8 aa, synthetic construct
IDPESGNT

SEQ ID NO: 93 - protein, 13 aa, synthetic construct
ARLDGSSYYAMDY

SEQ ID NO: 94 - protein, 10 aa, synthetic construct
ESVDSYGNSF

SEQ ID NO: 95 - skipped

SEQ ID NO: 96 - protein, 9 aa, synthetic construct
QQNNEDPLT

SEQ ID NO: 97 - protein, 5 aa, synthetic construct
DYYMH

SEQ ID NO: 98 - protein, 17 aa, synthetic construct
WIDPESGNTIYDPKFQG

SEQ ID NO: 99 - protein, 11 aa, synthetic construct
LDGSSYYAMDY

SEQ ID NO: 100 - protein, 15 aa, synthetic construct
RASESVDSYGNSFMH

SEQ ID NO: 101 - protein, 7 aa, synthetic construct
LASNLES

SEQ ID NO: 102 - protein, 9 aa, synthetic construct
QQNNEDPLT

SEQ ID NO: 103 - protein, 7 aa, synthetic construct
GFNIKDY

SEQ ID NO: 104 - protein, 6 aa, synthetic construct
DPESGN

SEQ ID NO: 105 - protein, 11 aa, synthetic construct
LDGSSYYAMDY

SEQ ID NO: 106 - protein, 15 aa, synthetic construct
RASESVDSYGNSFMH

SEQ ID NO: 107 - protein, 7 aa, synthetic construct
LASNLES

SEQ ID NO: 108 - protein, 9 aa, synthetic construct
QQNNEDPLT

SEQ ID NO: 109 - protein, 13 aa, synthetic construct
KASGFNIKDYYMH

SEQ ID NO: 110 - protein, 10 aa, synthetic construct
WIDPESGNTI

SEQ ID NO: 111 - protein, 13 aa, synthetic construct
ARLDGSSYYAMDY

SEQ ID NO: 112 - protein, 15 aa, synthetic construct
RASESVDSYGNSFMH

SEQ ID NO: 113 - protein, 8 aa, synthetic construct
YLASNLES

SEQ ID NO: 114 - protein, 9 aa, synthetic construct
QQNNEDPLT

SEQ ID NO: 115 - protein, 8 aa, synthetic construct
GFNIKDYY

SEQ ID NO: 116 - protein, 8 aa, synthetic construct
IDPESGNT

SEQ ID NO: 117 - protein, 13 aa, synthetic construct
ARLDGSSYYAMDY

SEQ ID NO: 118 - protein, 10 aa, synthetic construct
ESVDSYGNSF

SEQ ID NO: 119 - skipped

SEQ ID NO: 120 - protein, 9 aa, synthetic construct
QQNNEDPLT

SEQ ID NO: 121 - protein, 8 aa, synthetic construct
GYTFSSSW

SEQ ID NO: 122 - protein, 8 aa, synthetic construct
ILPGSGST

SEQ ID NO: 123 - protein, 15 aa, synthetic construct
ARSGAGGYDSGAFAY

SEQ ID NO: 124 - protein, 10 aa, synthetic construct
ESIDTYGNSF

SEQ ID NO: 125 - skipped

SEQ ID NO: 126 - protein, 9 aa, synthetic construct
QQSNEDPWT

SEQ ID NO: 127 - protein, 5 aa, synthetic construct
SSWTE

SEQ ID NO: 128 - protein, 17 aa, synthetic construct
EILPGSGSTNYNEKFKG

SEQ ID NO: 129 - protein, 13 aa, synthetic construct
SGAGGYDSGAFAY

SEQ ID NO: 130 - protein, 15 aa, synthetic construct
RASESIDTYGNSFMH

SEQ ID NO: 131 - protein, 7 aa, synthetic construct
RTSNLES

SEQ ID NO: 132 - protein, 9 aa, synthetic construct
QQSNEDPWT

SEQ ID NO: 133 - protein, 7 aa, synthetic construct
GYTFSSS

SEQ ID NO: 134 - protein, 6 aa, synthetic construct
LPGSGS

SEQ ID NO: 135 - protein, 13 aa, synthetic construct
SGAGGYDSGAFAY

SEQ ID NO: 136 - protein, 15 aa, synthetic construct
RASESIDTYGNSFMH

SEQ ID NO: 137 - protein, 7 aa, synthetic construct
RTSNLES

SEQ ID NO: 138 - protein, 9 aa, synthetic construct
QQSNEDPWT

SEQ ID NO: 139 - protein, 13 aa, synthetic construct
KATGYTFSSSWTE

SEQ ID NO: 140 - protein, 10 aa, synthetic construct
EILPGSGSTN

SEQ ID NO: 141 - protein, 15 aa, synthetic construct
ARSGAGGYDSGAFAY

SEQ ID NO: 142 - protein, 15 aa, synthetic construct
RASESIDTYGNSFMH

SEQ ID NO: 143 - protein, 8 aa, synthetic construct
YRTSNLES

SEQ ID NO: 144 - protein, 9 aa, synthetic construct
QQSNEDPWT

SEQ ID NO: 145 - protein, 8 aa, synthetic construct
GYTFSSSW

SEQ ID NO: 146 - protein, 8 aa, synthetic construct
ILPGSGST

SEQ ID NO: 147 - protein, 15 aa, synthetic construct
ARSGAGGYDSGAFAY

SEQ ID NO: 148 - protein, 10 aa, synthetic construct
ESIDTYGNSF

SEQ ID NO: 149 - skipped

SEQ ID NO: 150 - protein, 9 aa, synthetic construct
QQSNEDPWT

SEQ ID NO: 151 - protein, 8 aa, synthetic construct
GYTFSNYW

SEQ ID NO: 152 - protein, 8 aa, synthetic construct
ILPGSGST

SEQ ID NO: 153 - protein, 15 aa, synthetic construct
ARSGAGGYDSGAFAY

SEQ ID NO: 154 - protein, 10 aa, synthetic construct
ESVDTYGNSF

SEQ ID NO: 155 - skipped

SEQ ID NO: 156 - protein, 9 aa, synthetic construct
QQSNEDPWT

SEQ ID NO: 157 - protein, 5 aa, synthetic construct
NYWIE

SEQ ID NO: 158 - protein, 17 aa, synthetic construct
EILPGSGSTDYNEKFEG

SEQ ID NO: 159 - protein, 13 aa, synthetic construct
SGAGGYDSGAFAY

SEQ ID NO: 160 - protein, 15 aa, synthetic construct
RASESVDTYGNSFMH

SEQ ID NO: 161 - protein, 7 aa, synthetic construct
RTSNLES

SEQ ID NO: 162 - protein, 9 aa, synthetic construct
QQSNEDPWT

SEQ ID NO: 163 - protein, 7 aa, synthetic construct
GYTFSNY

SEQ ID NO: 164 - protein, 6 aa, synthetic construct
LPGSGS

SEQ ID NO: 165 - protein, 13 aa, synthetic construct
SGAGGYDSGAFAY

SEQ ID NO: 166 - protein, 15 aa, synthetic construct
RASESVDTYGNSFMH

SEQ ID NO: 167 - protein, 7 aa, synthetic construct
RTSNLES

SEQ ID NO: 168 - protein, 9 aa, synthetic construct
QQSNEDPWT

SEQ ID NO: 169 - protein, 13 aa, synthetic construct
KATGYTFSNYWIE

SEQ ID NO: 170 - protein, 10 aa, synthetic construct
EILPGSGSTD

SEQ ID NO: 171 - protein, 15 aa, synthetic construct
ARSGAGGYDSGAFAY

SEQ ID NO: 172 - protein, 15 aa, synthetic construct
RASESVDTYGNSFMH

SEQ ID NO: 173 - protein, 8 aa, synthetic construct
YRTSNLES

SEQ ID NO: 174 - protein, 9 aa, synthetic construct
QQSNEDPWT

SEQ ID NO: 175 - protein, 8 aa, synthetic construct
GYTFSNYW

SEQ ID NO: 176 - protein, 8 aa, synthetic construct
ILPGSGST

SEQ ID NO: 177 - protein, 15 aa, synthetic construct
ARSGAGGYDSGAFAY

SEQ ID NO: 178 - protein, 10 aa, synthetic construct
ESVDTYGNSF

SEQ ID NO: 179 - skipped

SEQ ID NO: 180 - protein, 9 aa, synthetic construct
QQSNEDPWT

SEQ ID NO: 181 - protein, 8 aa, synthetic construct
GYTFSNYW

SEQ ID NO: 182 - protein, 8 aa, synthetic construct
ILPGSGST

SEQ ID NO: 183 - protein, 15 aa, synthetic construct
ARSGAGGYDSGAFAY

SEQ ID NO: 184 - protein, 10 aa, synthetic construct
ESVDSYGNSF

SEQ ID NO: 185 - skipped

SEQ ID NO: 186 - protein, 9 aa, synthetic construct
QQSNEDPWT

SEQ ID NO: 187 - protein, 5 aa, synthetic construct
NYWIE

SEQ ID NO: 188 - protein, 17 aa, synthetic construct
EILPGSGSTNYNEKFKG

SEQ ID NO: 189 - protein, 13 aa, synthetic construct
SGAGGYDSGAFAY

SEQ ID NO: 190 - protein, 15 aa, synthetic construct
RASESVDSYGNSFMH

SEQ ID NO: 191 - protein, 7 aa, synthetic construct
RTSNLES

SEQ ID NO: 192 - protein, 9 aa, synthetic construct
QQSNEDPWT

SEQ ID NO: 193 - protein, 7 aa, synthetic construct
GYTFSNY

SEQ ID NO: 194 - protein, 6 aa, synthetic construct
LPGSGS

SEQ ID NO: 195 - protein, 13 aa, synthetic construct
SGAGGYDSGAFAY

SEQ ID NO: 196 - protein, 15 aa, synthetic construct
RASESVDSYGNSFMH

SEQ ID NO: 197 - protein, 7 aa, synthetic construct
RTSNLES

SEQ ID NO: 198 - protein, 9 aa, synthetic construct
QQSNEDPWT

SEQ ID NO: 199 - protein, 13 aa, synthetic construct
KATGYTFSNYWIE

SEQ ID NO: 200 - protein, 10 aa, synthetic construct
EILPGSGSTN

SEQ ID NO: 201 - protein, 15 aa, synthetic construct
ARSGAGGYDSGAFAY

SEQ ID NO: 202 - protein, 15 aa, synthetic construct
RASESVDSYGNSFMH

SEQ ID NO: 203 - protein, 8 aa, synthetic construct
YRTSNLES

SEQ ID NO: 204 - protein, 9 aa, synthetic construct
QQSNEDPWT

SEQ ID NO: 205 - protein, 8 aa, synthetic construct
GYTFSNYW

SEQ ID NO: 206 - protein, 8 aa, synthetic construct
ILPGSGST

SEQ ID NO: 207 - protein, 15 aa, synthetic construct
ARSGAGGYDSGAFAY

SEQ ID NO: 208 - protein, 10 aa, synthetic construct
ESVDSYGNSF

SEQ ID NO: 209 - skipped

SEQ ID NO: 210 - protein, 9 aa, synthetic construct
QQSNEDPWT

SEQ ID NO: 211 - protein, 8 aa, synthetic construct
GNTFSSYW

SEQ ID NO: 212 - protein, 8 aa, synthetic construct
ILPGSGST

SEQ ID NO: 213 - protein, 15 aa, synthetic construct
ARSGAGGYDSGAFAY

SEQ ID NO: 214 - protein, 10 aa, synthetic construct
ESIDSYGNSF

SEQ ID NO: 215 - skipped

SEQ ID NO: 216 - protein, 9 aa, synthetic construct
QQSNEDPWT

SEQ ID NO: 217 - protein, 5 aa, synthetic construct
SYWIE

SEQ ID NO: 218 - protein, 17 aa, synthetic construct
EILPGSGSTNYNEKFKG

SEQ ID NO: 219 - protein, 13 aa, synthetic construct
SGAGGYDSGAFAY

SEQ ID NO: 220 - protein, 15 aa, synthetic construct
RASESIDSYGNSFMH

SEQ ID NO: 221 - protein, 7 aa, synthetic construct
RTSNLES

SEQ ID NO: 222 - protein, 9 aa, synthetic construct
QQSNEDPWT

SEQ ID NO: 223 - protein, 7 aa, synthetic construct
GNTFSSY

SEQ ID NO: 224 - protein, 6 aa, synthetic construct
LPGSGS

SEQ ID NO: 225 - protein, 13 aa, synthetic construct
SGAGGYDSGAFAY

SEQ ID NO: 226 - protein, 15 aa, synthetic construct
RASESIDSYGNSFMH

SEQ ID NO: 227 - protein, 7 aa, synthetic construct
RTSNLES

SEQ ID NO: 228 - protein, 9 aa, synthetic construct
QQSNEDPWT

SEQ ID NO: 229 - protein, 13 aa, synthetic construct
KATGNTFSSYWIE

SEQ ID NO: 230 - protein, 10 aa, synthetic construct
EILPGSGSTN

SEQ ID NO: 231 - protein, 15 aa, synthetic construct
ARSGAGGYDSGAFAY

SEQ ID NO: 232 - protein, 15 aa, synthetic construct
RASESIDSYGNSFMH

SEQ ID NO: 233 - protein, 8 aa, synthetic construct
YRTSNLES

SEQ ID NO: 234 - protein, 9 aa, synthetic construct
QQSNEDPWT

SEQ ID NO: 235 - protein, 8 aa, synthetic construct
GNTFSSYW

SEQ ID NO: 236 - protein, 8 aa, synthetic construct
ILPGSGST

SEQ ID NO: 237 - protein, 15 aa, synthetic construct
ARSGAGGYDSGAFAY

SEQ ID NO: 238 - protein, 10 aa, synthetic construct
ESIDSYGNSF

SEQ ID NO: 239 - skipped

SEQ ID NO: 240 - protein, 9 aa, synthetic construct
QQSNEDPWT

SEQ ID NO: 241 - protein, 8 aa, synthetic construct
GNTFSSYW

SEQ ID NO: 242 - protein, 8 aa, synthetic construct
ILPGSGSS

SEQ ID NO: 243 - protein, 15 aa, synthetic construct
ARSGAGGYDSGAFAY

SEQ ID NO: 244 - protein, 10 aa, synthetic construct
ESVDSYGNSF

SEQ ID NO: 245 - skipped

SEQ ID NO: 246 - protein, 9 aa, synthetic construct
QQSNEDPWT

SEQ ID NO: 247 - protein, 5 aa, synthetic construct
SYWIE

SEQ ID NO: 248 - protein, 17 aa, synthetic construct
EILPGSGSSNYNEKFKG

SEQ ID NO: 249 - protein, 13 aa, synthetic construct
SGAGGYDSGAFAY

SEQ ID NO: 250 - protein, 15 aa, synthetic construct
RASESVDSYGNSFMH

SEQ ID NO: 251 - protein, 7 aa, synthetic construct
RTSNLES

SEQ ID NO: 252 - protein, 9 aa, synthetic construct
QQSNEDPWT

SEQ ID NO: 253 - protein, 7 aa, synthetic construct
GNTFSSY

SEQ ID NO: 254 - protein, 6 aa, synthetic construct
LPGSGS

SEQ ID NO: 255 - protein, 13 aa, synthetic construct
SGAGGYDSGAFAY

SEQ ID NO: 256 - protein, 15 aa, synthetic construct
RASESVDSYGNSFMH

SEQ ID NO: 257 - protein, 7 aa, synthetic construct
RTSNLES

SEQ ID NO: 258 - protein, 9 aa, synthetic construct
QQSNEDPWT

SEQ ID NO: 259 - protein, 13 aa, synthetic construct
KTTGNTFSSYWIE

SEQ ID NO: 260 - protein, 10 aa, synthetic construct
EILPGSGSSN

SEQ ID NO: 261 - protein, 15 aa, synthetic construct
ARSGAGGYDSGAFAY

SEQ ID NO: 262 - protein, 15 aa, synthetic construct
RASESVDSYGNSFMH

SEQ ID NO: 263 - protein, 8 aa, synthetic construct
YRTSNLES

SEQ ID NO: 264 - protein, 9 aa, synthetic construct
QQSNEDPWT

SEQ ID NO: 265 - protein, 8 aa, synthetic construct
GNTFSSYW

SEQ ID NO: 266 - protein, 8 aa, synthetic construct
ILPGSGSS

SEQ ID NO: 267 - protein, 15 aa, synthetic construct
ARSGAGGYDSGAFAY

SEQ ID NO: 268 - protein, 10 aa, synthetic construct
ESVDSYGNSF

SEQ ID NO: 269 - skipped

SEQ ID NO: 270 - protein, 9 aa, synthetic construct
QQSNEDPWT

SEQ ID NO: 271 - protein, 8 aa, synthetic construct
GNTFSSYW

SEQ ID NO: 272 - protein, 8 aa, synthetic construct
ILPGSGST

SEQ ID NO: 273 - protein, 15 aa, synthetic construct
ARSGAGGYDSGAFAY

SEQ ID NO: 274 - protein, 10 aa, synthetic construct
ESVDSYGNSF

SEQ ID NO: 275 - skipped

SEQ ID NO: 276 - protein, 9 aa, synthetic construct
QQSNEDPWT

SEQ ID NO: 277 - protein, 5 aa, synthetic construct
SYWIE

SEQ ID NO: 278 - protein, 17 aa, synthetic construct
EILPGSGSTNYNEKFKG

SEQ ID NO: 279 - protein, 13 aa, synthetic construct
SGAGGYDSGAFAY

SEQ ID NO: 280 - protein, 15 aa, synthetic construct
RASESVDSYGNSFMH

SEQ ID NO: 281 - protein, 7 aa, synthetic construct
RTSNLES

SEQ ID NO: 282 - protein, 9 aa, synthetic construct
QQSNEDPWT

SEQ ID NO: 283 - protein, 7 aa, synthetic construct
GNTFSSY

SEQ ID NO: 284 - protein, 6 aa, synthetic construct
LPGSGS

SEQ ID NO: 285 - protein, 13 aa, synthetic construct
SGAGGYDSGAFAY

SEQ ID NO: 286 - protein, 15 aa, synthetic construct
RASESVDSYGNSFMH

SEQ ID NO: 287 - protein, 7 aa, synthetic construct
RTSNLES

SEQ ID NO: 288 - protein, 9 aa, synthetic construct
QQSNEDPWT

SEQ ID NO: 289 - protein, 13 aa, synthetic construct
KTTGNTFSSYWIE

SEQ ID NO: 290 - protein, 10 aa, synthetic construct
EILPGSGSTN

SEQ ID NO: 291 - protein, 15 aa, synthetic construct
ARSGAGGYDSGAFAY

SEQ ID NO: 292 - protein, 15 aa, synthetic construct
RASESVDSYGNSFMH

SEQ ID NO: 293 - protein, 8 aa, synthetic construct
YRTSNLES

SEQ ID NO: 294 - protein, 9 aa, synthetic construct
QQSNEDPWT

SEQ ID NO: 295 - protein, 8 aa, synthetic construct
GNTFSSYW

SEQ ID NO: 296 - protein, 8 aa, synthetic construct
ILPGSGST

SEQ ID NO: 297 - protein, 15 aa, synthetic construct
ARSGAGGYDSGAFAY

SEQ ID NO: 298 - protein, 10 aa, synthetic construct
ESVDSYGNSF

SEQ ID NO: 299 - skipped

SEQ ID NO: 300 - protein, 9 aa, synthetic construct
QQSNEDPWT

SEQ ID NO: 301 - protein, 8 aa, synthetic construct
GNTFSNYW

SEQ ID NO: 302 - protein, 8 aa, synthetic construct
ILPGSGST

SEQ ID NO: 303 - protein, 15 aa, synthetic construct
ARSGAGGYDSGAFAY

SEQ ID NO: 304 - protein, 10 aa, synthetic construct
ESVDSYGNSF

SEQ ID NO: 305 - skipped

SEQ ID NO: 306 - protein, 9 aa, synthetic construct
QQSNEDPWT

SEQ ID NO: 307 - protein, 5 aa, synthetic construct
NYWIE

SEQ ID NO: 308 - protein, 17 aa, synthetic construct
EILPGSGSTNYNEKFKG

SEQ ID NO: 309 - protein, 13 aa, synthetic construct
SGAGGYDSGAFAY

SEQ ID NO: 310 - protein, 15 aa, synthetic construct
RASESVDSYGNSFMH

SEQ ID NO: 311 - protein, 7 aa, synthetic construct
RTSNLES

SEQ ID NO: 312 - protein, 9 aa, synthetic construct
QQSNEDPWT

SEQ ID NO: 313 - protein, 7 aa, synthetic construct
GNTFSNY

SEQ ID NO: 314 - protein, 6 aa, synthetic construct
LPGSGS

SEQ ID NO: 315 - protein, 13 aa, synthetic construct
SGAGGYDSGAFAY

SEQ ID NO: 316 - protein, 15 aa, synthetic construct
RASESVDSYGNSFMH

SEQ ID NO: 317 - protein, 7 aa, synthetic construct
RTSNLES

SEQ ID NO: 318 - protein, 9 aa, synthetic construct
QQSNEDPWT

SEQ ID NO: 319 - protein, 13 aa, synthetic construct
KATGNTFSNYWIE

SEQ ID NO: 320 - protein, 10 aa, synthetic construct
EILPGSGSTN

SEQ ID NO: 321 - protein, 15 aa, synthetic construct
ARSGAGGYDSGAFAY

SEQ ID NO: 322 - protein, 15 aa, synthetic construct
RASESVDSYGNSFMH

SEQ ID NO: 323 - protein, 8 aa, synthetic construct
YRTSNLES

SEQ ID NO: 324 - protein, 9 aa, synthetic construct
QQSNEDPWT

SEQ ID NO: 325 - protein, 8 aa, synthetic construct
GNTFSNYW

SEQ ID NO: 326 - protein, 8 aa, synthetic construct
ILPGSGST

SEQ ID NO: 327 - protein, 15 aa, synthetic construct
ARSGAGGYDSGAFAY

SEQ ID NO: 328 - protein, 10 aa, synthetic construct
ESVDSYGNSF

SEQ ID NO: 329 - skipped

SEQ ID NO: 330 - protein, 9 aa, synthetic construct
QQSNEDPWT

SEQ ID NO: 331 - protein, 8 aa, synthetic construct
GSTFSSYW

SEQ ID NO: 332 - protein, 8 aa, synthetic construct
ILPGSGST

SEQ ID NO: 333 - protein, 15 aa, synthetic construct
ARSGAGGYDSGAFAY

SEQ ID NO: 334 - protein, 10 aa, synthetic construct
ESVDTYGNSF

SEQ ID NO: 335 - skipped

SEQ ID NO: 336 - protein, 9 aa, synthetic construct
QQSNEDPWT

SEQ ID NO: 337 - protein, 5 aa, synthetic construct
SYWIE

SEQ ID NO: 338 - protein, 17 aa, synthetic construct
EILPGSGSTNYNEKFKG

SEQ ID NO: 339 - protein, 13 aa, synthetic construct
SGAGGYDSGAFAY

SEQ ID NO: 340 - protein, 15 aa, synthetic construct
RASESVDTYGNSFMH

SEQ ID NO: 341 - protein, 7 aa, synthetic construct
RTSNLES

SEQ ID NO: 342 - protein, 9 aa, synthetic construct
QQSNEDPWT

SEQ ID NO: 343 - protein, 7 aa, synthetic construct
GSTFSSY

SEQ ID NO: 344 - protein, 6 aa, synthetic construct
LPGSGS

SEQ ID NO: 345 - protein, 13 aa, synthetic construct
SGAGGYDSGAFAY

SEQ ID NO: 346 - protein, 15 aa, synthetic construct
RASESVDTYGNSFMH

SEQ ID NO: 347 - protein, 7 aa, synthetic construct
RTSNLES

SEQ ID NO: 348 - protein, 9 aa, synthetic construct
QQSNEDPWT

SEQ ID NO: 349 - protein, 13 aa, synthetic construct
KATGSTFSSYWIE

SEQ ID NO: 350 - protein, 10 aa, synthetic construct
EILPGSGSTN

SEQ ID NO: 351 - protein, 15 aa, synthetic construct
ARSGAGGYDSGAFAY

SEQ ID NO: 352 - protein, 15 aa, synthetic construct
RASESVDTYGNSFMH

SEQ ID NO: 353 - protein, 8 aa, synthetic construct
YRTSNLES

SEQ ID NO: 354 - protein, 9 aa, synthetic construct
QQSNEDPWT

SEQ ID NO: 355 - protein, 8 aa, synthetic construct
GSTFSSYW

SEQ ID NO: 356 - protein, 8 aa, synthetic construct
ILPGSGST

SEQ ID NO: 357 - protein, 15 aa, synthetic construct
ARSGAGGYDSGAFAY

SEQ ID NO: 358 - protein, 10 aa, synthetic construct
ESVDTYGNSF

SEQ ID NO: 359 - skipped

SEQ ID NO: 360 - protein, 9 aa, synthetic construct
QQSNEDPWT

SEQ ID NO: 361 - protein, 8 aa, synthetic construct
GYTLSSYW

SEQ ID NO: 362 - protein, 8 aa, synthetic construct
ILPGSGST

SEQ ID NO: 363 - protein, 15 aa, synthetic construct
ARSGAGGYDSGAFAY

SEQ ID NO: 364 - protein, 10 aa, synthetic construct
ESVDTYGNSF

SEQ ID NO: 365 - skipped

SEQ ID NO: 366 - protein, 9 aa, synthetic construct
QQSNEDPWT

SEQ ID NO: 367 - protein, 5 aa, synthetic construct
SYWIE

SEQ ID NO: 368 - protein, 17 aa, synthetic construct
EILPGSGSTNYNEKFKG

SEQ ID NO: 369 - protein, 13 aa, synthetic construct
SGAGGYDSGAFAY

SEQ ID NO: 370 - protein, 15 aa, synthetic construct
RASESVDTYGNSFMH

SEQ ID NO: 371 - protein, 7 aa, synthetic construct
RTSNLES

SEQ ID NO: 372 - protein, 9 aa, synthetic construct
QQSNEDPWT

SEQ ID NO: 373 - protein, 7 aa, synthetic construct
GYTLSSY

SEQ ID NO: 374 - protein, 6 aa, synthetic construct
LPGSGS

SEQ ID NO: 375 - protein, 13 aa, synthetic construct
SGAGGYDSGAFAY

SEQ ID NO: 376 - protein, 15 aa, synthetic construct
RASESVDTYGNSFMH

SEQ ID NO: 377 - protein, 7 aa, synthetic construct
RTSNLES

SEQ ID NO: 378 - protein, 9 aa, synthetic construct
QQSNEDPWT

SEQ ID NO: 379 - protein, 13 aa, synthetic construct
KATGYTLSSYWIE

SEQ ID NO: 380 - protein, 10 aa, synthetic construct
EILPGSGSTN

SEQ ID NO: 381 - protein, 15 aa, synthetic construct
ARSGAGGYDSGAFAY

SEQ ID NO: 382 - protein, 15 aa, synthetic construct
RASESVDTYGNSFMH

SEQ ID NO: 383 - protein, 8 aa, synthetic construct
YRTSNLES

SEQ ID NO: 384 - protein, 9 aa, synthetic construct
QQSNEDPWT

SEQ ID NO: 385 - protein, 8 aa, synthetic construct
GYTLSSYW

SEQ ID NO: 386 - protein, 8 aa, synthetic construct
ILPGSGST

SEQ ID NO: 387 - protein, 15 aa, synthetic construct
ARSGAGGYDSGAFAY

SEQ ID NO: 388 - protein, 10 aa, synthetic construct
ESVDTYGNSF

SEQ ID NO: 389 - skipped

SEQ ID NO: 390 - protein, 9 aa, synthetic construct
QQSNEDPWT

SEQ ID NO: 391 - protein, 8 aa, synthetic construct
GYTFSRYW

SEQ ID NO: 392 - protein, 8 aa, synthetic construct
ILPGDGST

SEQ ID NO: 393 - protein, 15 aa, synthetic construct
ARSGAGGYDSGAFAY

SEQ ID NO: 394 - protein, 10 aa, synthetic construct
ESVDTYGNSF

SEQ ID NO: 395 - skipped

SEQ ID NO: 396 - protein, 9 aa, synthetic construct
QQSNEDPWT

SEQ ID NO: 397 - protein, 5 aa, synthetic construct
RYWIE

SEQ ID NO: 398 - protein, 17 aa, synthetic construct
EILPGDGSTNYNAKFKG

SEQ ID NO: 399 - protein, 13 aa, synthetic construct
SGAGGYDSGAFAY

SEQ ID NO: 400 - protein, 15 aa, synthetic construct
RASESVDTYGNSFMH

SEQ ID NO: 401 - protein, 7 aa, synthetic construct
RTSNLES

SEQ ID NO: 402 - protein, 9 aa, synthetic construct
QQSNEDPWT

SEQ ID NO: 403 - protein, 7 aa, synthetic construct
GYTFSRY

SEQ ID NO: 404 - protein, 6 aa, synthetic construct
LPGDGS

SEQ ID NO: 405 - protein, 13 aa, synthetic construct
SGAGGYDSGAFAY

SEQ ID NO: 406 - protein, 15 aa, synthetic construct
RASESVDTYGNSFMH

SEQ ID NO: 407 - protein, 7 aa, synthetic construct
RTSNLES

SEQ ID NO: 408 - protein, 9 aa, synthetic construct
QQSNEDPWT

SEQ ID NO: 409 - protein, 13 aa, synthetic construct
KATGYTFSRYWIE

SEQ ID NO: 410 - protein, 10 aa, synthetic construct
EILPGDGSTN

SEQ ID NO: 411 - protein, 15 aa, synthetic construct
ARSGAGGYDSGAFAY

SEQ ID NO: 412 - protein, 15 aa, synthetic construct
RASESVDTYGNSFMH

SEQ ID NO: 413 - protein, 8 aa, synthetic construct
YRTSNLES

SEQ ID NO: 414 - protein, 9 aa, synthetic construct
QQSNEDPWT

SEQ ID NO: 415 - protein, 8 aa, synthetic construct
GYTFSRYW

SEQ ID NO: 416 - protein, 8 aa, synthetic construct
ILPGDGST

SEQ ID NO: 417 - protein, 15 aa, synthetic construct
ARSGAGGYDSGAFAY

SEQ ID NO: 418 - protein, 10 aa, synthetic construct
ESVDTYGNSF

SEQ ID NO: 419 - skipped

SEQ ID NO: 420 - protein, 9 aa, synthetic construct
QQSNEDPWT

SEQ ID NO: 421 - protein, 8 aa, synthetic construct
GYTFSSYW

SEQ ID NO: 422 - protein, 8 aa, synthetic construct
ILPGDGST

SEQ ID NO: 423 - protein, 15 aa, synthetic construct
ARSGAGGYDSGAFAY

SEQ ID NO: 424 - protein, 10 aa, synthetic construct
ESVDTYGNSF

SEQ ID NO: 425 - skipped

SEQ ID NO: 426 - protein, 9 aa, synthetic construct
QQSNEDPWT

SEQ ID NO: 427 - protein, 5 aa, synthetic construct
SYWIE

SEQ ID NO: 428 - protein, 17 aa, synthetic construct
EILPGDGSTNYNAKFKG

SEQ ID NO: 429 - protein, 13 aa, synthetic construct
SGAGGYDSGAFAY

SEQ ID NO: 430 - protein, 15 aa, synthetic construct
RASESVDTYGNSFMH

SEQ ID NO: 431 - protein, 7 aa, synthetic construct
RTSNLES

SEQ ID NO: 432 - protein, 9 aa, synthetic construct
QQSNEDPWT

SEQ ID NO: 433 - protein, 7 aa, synthetic construct
GYTFSSY

SEQ ID NO: 434 - protein, 6 aa, synthetic construct
LPGDGS

SEQ ID NO: 435 - protein, 13 aa, synthetic construct
SGAGGYDSGAFAY

SEQ ID NO: 436 - protein, 15 aa, synthetic construct
RASESVDTYGNSFMH

SEQ ID NO: 437 - protein, 7 aa, synthetic construct
RTSNLES

SEQ ID NO: 438 - protein, 9 aa, synthetic construct
QQSNEDPWT

SEQ ID NO: 439 - protein, 13 aa, synthetic construct
KATGYTFSSYWIE

SEQ ID NO: 440 - protein, 10 aa, synthetic construct
EILPGDGSTN

SEQ ID NO: 441 - protein, 15 aa, synthetic construct
ARSGAGGYDSGAFAY

SEQ ID NO: 442 - protein, 15 aa, synthetic construct
RASESVDTYGNSFMH

SEQ ID NO: 443 - protein, 8 aa, synthetic construct
YRTSNLES

SEQ ID NO: 444 - protein, 9 aa, synthetic construct
QQSNEDPWT

SEQ ID NO: 445 - protein, 8 aa, synthetic construct
GYTFSSYW

SEQ ID NO: 446 - protein, 8 aa, synthetic construct
ILPGDGST

SEQ ID NO: 447 - protein, 15 aa, synthetic construct
ARSGAGGYDSGAFAY

SEQ ID NO: 448 - protein, 10 aa, synthetic construct
ESVDTYGNSF

SEQ ID NO: 449 - skipped

SEQ ID NO: 450 - protein, 9 aa, synthetic construct
QQSNEDPWT

SEQ ID NO: 451 - protein, 8 aa, synthetic construct
GYTFSSYW

SEQ ID NO: 452 - protein, 8 aa, synthetic construct
ILPGSGST

SEQ ID NO: 453 - protein, 15 aa, synthetic construct
ARSGAGGYDSGAFAY

SEQ ID NO: 454 - protein, 10 aa, synthetic construct
ESVDTYGNSF

SEQ ID NO: 455 - skipped

SEQ ID NO: 456 - protein, 9 aa, synthetic construct
QQSNEDPWT

SEQ ID NO: 457 - protein, 5 aa, synthetic construct
SYWIE

SEQ ID NO: 458 - protein, 17 aa, synthetic construct
EILPGSGSTNYNEKFKG

SEQ ID NO: 459 - protein, 13 aa, synthetic construct
SGAGGYDSGAFAY

SEQ ID NO: 460 - protein, 15 aa, synthetic construct
RASESVDTYGNSFMH

SEQ ID NO: 461 - protein, 7 aa, synthetic construct
RTSNLES

SEQ ID NO: 462 - protein, 9 aa, synthetic construct
QQSNEDPWT

SEQ ID NO: 463 - protein, 7 aa, synthetic construct
GYTFSSY

SEQ ID NO: 464 - protein, 6 aa, synthetic construct
LPGSGS

SEQ ID NO: 465 - protein, 13 aa, synthetic construct
SGAGGYDSGAFAY

SEQ ID NO: 466 - protein, 15 aa, synthetic construct
RASESVDTYGNSFMH

SEQ ID NO: 467 - protein, 7 aa, synthetic construct
RTSNLES

SEQ ID NO: 468 - protein, 9 aa, synthetic construct
QQSNEDPWT

SEQ ID NO: 469 - protein, 13 aa, synthetic construct
KITGYTFSSYWIE

SEQ ID NO: 470 - protein, 10 aa, synthetic construct
EILPGSGSTN

SEQ ID NO: 471 - protein, 15 aa, synthetic construct
ARSGAGGYDSGAFAY

SEQ ID NO: 472 - protein, 15 aa, synthetic construct
RASESVDTYGNSFMH

SEQ ID NO: 473 - protein, 8 aa, synthetic construct
YRTSNLES

SEQ ID NO: 474 - protein, 9 aa, synthetic construct
QQSNEDPWT

SEQ ID NO: 475 - protein, 8 aa, synthetic construct
GYTFSSYW

SEQ ID NO: 476 - protein, 8 aa, synthetic construct
ILPGSGST

SEQ ID NO: 477 - protein, 15 aa, synthetic construct
ARSGAGGYDSGAFAY

SEQ ID NO: 478 - protein, 10 aa, synthetic construct
ESVDTYGNSF

SEQ ID NO: 479 - skipped

SEQ ID NO: 480 - protein, 9 aa, synthetic construct
QQSNEDPWT

SEQ ID NO: 481 - protein, 8 aa, synthetic construct
GYTFSSYW

SEQ ID NO: 482 - protein, 8 aa, synthetic construct
ILPGSGST

SEQ ID NO: 483 - protein, 15 aa, synthetic construct
ARSGAGGYDSGAFAY

SEQ ID NO: 484 - protein, 10 aa, synthetic construct
ESVDTYGNSF

SEQ ID NO: 485 - skipped

SEQ ID NO: 486 - protein, 9 aa, synthetic construct
QQSNEDPWT

SEQ ID NO: 487 - protein, 5 aa, synthetic construct
SYWIE

SEQ ID NO: 488 - protein, 17 aa, synthetic construct
EILPGSGSTNYNEKFKG

SEQ ID NO: 489 - protein, 13 aa, synthetic construct
SGAGGYDSGAFAY

SEQ ID NO: 490 - protein, 15 aa, synthetic construct
RASESVDTYGNSFMH

SEQ ID NO: 491 - protein, 7 aa, synthetic construct
RTSNLES

SEQ ID NO: 492 - protein, 9 aa, synthetic construct
QQSNEDPWT

SEQ ID NO: 493 - protein, 7 aa, synthetic construct
GYTFSSY

SEQ ID NO: 494 - protein, 6 aa, synthetic construct
LPGSGS

SEQ ID NO: 495 - protein, 13 aa, synthetic construct
SGAGGYDSGAFAY

SEQ ID NO: 496 - protein, 15 aa, synthetic construct
RASESVDTYGNSFMH

SEQ ID NO: 497 - protein, 7 aa, synthetic construct
RTSNLES

SEQ ID NO: 498 - protein, 9 aa, synthetic construct
QQSNEDPWT

SEQ ID NO: 499 - protein, 13 aa, synthetic construct
KATGYTFSSYWIE

SEQ ID NO: 500 - protein, 10 aa, synthetic construct
EILPGSGSTN

SEQ ID NO: 501 - protein, 15 aa, synthetic construct
ARSGAGGYDSGAFAY

SEQ ID NO: 502 - protein, 15 aa, synthetic construct
RASESVDTYGNSFMH

SEQ ID NO: 503 - protein, 8 aa, synthetic construct
YRTSNLES

SEQ ID NO: 504 - protein, 9 aa, synthetic construct
QQSNEDPWT

SEQ ID NO: 505 - protein, 8 aa, synthetic construct
GYTFSSYW

SEQ ID NO: 506 - protein, 8 aa, synthetic construct
ILPGSGST

SEQ ID NO: 507 - protein, 15 aa, synthetic construct
ARSGAGGYDSGAFAY

SEQ ID NO: 508 - protein, 10 aa, synthetic construct
ESVDTYGNSF

SEQ ID NO: 509 - skipped

SEQ ID NO: 510 - protein, 9 aa, synthetic construct
QQSNEDPWT

SEQ ID NO: 511 - protein, 8 aa, synthetic construct
GYTFSSYW

SEQ ID NO: 512 - protein, 8 aa, synthetic construct
ILPGSGST

SEQ ID NO: 513 - protein, 15 aa, synthetic construct
ARSGAGGYDSGAFGY

SEQ ID NO: 514 - protein, 10 aa, synthetic construct
ESIDTYGNSF

SEQ ID NO: 515 - skipped

SEQ ID NO: 516 - protein, 9 aa, synthetic construct
QQSNEDPWT

SEQ ID NO: 517 - protein, 5 aa, synthetic construct
SYWIE

SEQ ID NO: 518 - protein, 17 aa, synthetic construct
EILPGSGSTNYNEKFKG

SEQ ID NO: 519 - protein, 13 aa, synthetic construct
SGAGGYDSGAFGY

SEQ ID NO: 520 - protein, 15 aa, synthetic construct
RASESIDTYGNSFMH

SEQ ID NO: 521 - protein, 7 aa, synthetic construct
RASNLES

SEQ ID NO: 522 - protein, 9 aa, synthetic construct
QQSNEDPWT

SEQ ID NO: 523 - protein, 7 aa, synthetic construct
GYTFSSY

SEQ ID NO: 524 - protein, 6 aa, synthetic construct
LPGSGS

SEQ ID NO: 525 - protein, 13 aa, synthetic construct
SGAGGYDSGAFGY

SEQ ID NO: 526 - protein, 15 aa, synthetic construct
RASESIDTYGNSFMH

SEQ ID NO: 527 - protein, 7 aa, synthetic construct
RASNLES

SEQ ID NO: 528 - protein, 9 aa, synthetic construct
QQSNEDPWT

SEQ ID NO: 529 - protein, 13 aa, synthetic construct
KATGYTFSSYWIE

SEQ ID NO: 530 - protein, 10 aa, synthetic construct
EILPGSGSTN

SEQ ID NO: 531 - protein, 15 aa, synthetic construct
ARSGAGGYDSGAFGY

SEQ ID NO: 532 - protein, 15 aa, synthetic construct
RASESIDTYGNSFMH

SEQ ID NO: 533 - protein, 8 aa, synthetic construct
YRASNLES

SEQ ID NO: 534 - protein, 9 aa, synthetic construct
QQSNEDPWT

SEQ ID NO: 535 - protein, 8 aa, synthetic construct
GYTFSSYW

SEQ ID NO: 536 - protein, 8 aa, synthetic construct
ILPGSGST

SEQ ID NO: 537 - protein, 15 aa, synthetic construct
ARSGAGGYDSGAFGY

SEQ ID NO: 538 - protein, 10 aa, synthetic construct
ESIDTYGNSF

SEQ ID NO: 539 - skipped

SEQ ID NO: 540 - protein, 9 aa, synthetic construct
QQSNEDPWT

SEQ ID NO: 541 - protein, 8 aa, synthetic construct
GYTFSSSW

SEQ ID NO: 542 - protein, 8 aa, synthetic construct
ILPGSGTT

SEQ ID NO: 543 - protein, 15 aa, synthetic construct
ARSGAGGYDSGAFTY

SEQ ID NO: 544 - protein, 10 aa, synthetic construct
ESIDTYGNSF

SEQ ID NO: 545 - skipped

SEQ ID NO: 546 - protein, 9 aa, synthetic construct
QQNNEDPWT

SEQ ID NO: 547 - protein, 5 aa, synthetic construct
SSWIE

SEQ ID NO: 548 - protein, 17 aa, synthetic construct
EILPGSGTTNYNEKFKG

SEQ ID NO: 549 - protein, 13 aa, synthetic construct
SGAGGYDSGAFTY

SEQ ID NO: 550 - protein, 15 aa, synthetic construct
RASESIDTYGNSFMH

SEQ ID NO: 551 - protein, 7 aa, synthetic construct
RASNLES

SEQ ID NO: 552 - protein, 9 aa, synthetic construct
QQSNEDPWT

SEQ ID NO: 553 - protein, 7 aa, synthetic construct
GYTFSSS

SEQ ID NO: 554 - protein, 6 aa, synthetic construct
LPGSGT

SEQ ID NO: 555 - protein, 13 aa, synthetic construct
SGAGGYDSGAFTY

SEQ ID NO: 556 - protein, 15 aa, synthetic construct
RASESIDTYGNSFMH

SEQ ID NO: 557 - protein, 7 aa, synthetic construct
RASNLES

SEQ ID NO: 558 - protein, 9 aa, synthetic construct
QQSNEDPWT

SEQ ID NO: 559 - protein, 13 aa, synthetic construct
KATGYTFSSSWIE

SEQ ID NO: 560 - protein, 10 aa, synthetic construct
EILPGSGTTN

SEQ ID NO: 561 - protein, 15 aa, synthetic construct
ARSGAGGYDSGAFTY

SEQ ID NO: 562 - protein, 15 aa, synthetic construct
RASESIDTYGNSFMH

SEQ ID NO: 563 - protein, 8 aa, synthetic construct
YRASNLES

SEQ ID NO: 564 - protein, 9 aa, synthetic construct
QQSNEDPWT

SEQ ID NO: 565 - protein, 8 aa, synthetic construct
GYTFSSSW

SEQ ID NO: 566 - protein, 8 aa, synthetic construct
ILPGSGTT

SEQ ID NO: 567 - protein, 15 aa, synthetic construct
ARSGAGGYDSGAFTY

SEQ ID NO: 568 - protein, 10 aa, synthetic construct
ESIDTYGNSF

SEQ ID NO: 569 - skipped

SEQ ID NO: 570 - protein, 9 aa, synthetic construct
QQSNEDPWT

SEQ ID NO: 571 - protein, 8 aa, synthetic construct
GYTFSSSW

SEQ ID NO: 572 - protein, 8 aa, synthetic construct
ILPGSGST

SEQ ID NO: 573 - protein, 15 aa, synthetic construct
ARSGAGGYDSGAFTY

SEQ ID NO: 574 - protein, 10 aa, synthetic construct
ESIDTYGNSF

SEQ ID NO: 575 - skipped

SEQ ID NO: 576 - protein, 9 aa, synthetic construct
QQSNEDPWT

SEQ ID NO: 577 - protein, 5 aa, synthetic construct
SSWIE

SEQ ID NO: 578 - protein, 17 aa, synthetic construct
EILPGSGSTNYNEKFKG

SEQ ID NO: 579 - protein, 13 aa, synthetic construct
SGAGGYDSGAFTY

SEQ ID NO: 580 - protein, 15 aa, synthetic construct
RASESIDTYGNSFMH

SEQ ID NO: 581 - protein, 7 aa, synthetic construct
RASNLES

SEQ ID NO: 582 - protein, 9 aa, synthetic construct
QQSNEDPWT

SEQ ID NO: 583 - protein, 7 aa, synthetic construct
GYTFSSS

SEQ ID NO: 584 - protein, 6 aa, synthetic construct
LPGSGS

SEQ ID NO: 585 - protein, 13 aa, synthetic construct
SGAGGYDSGAFTY

SEQ ID NO: 586 - protein, 15 aa, synthetic construct
RASESIDTYGNSFMH

SEQ ID NO: 587 - protein, 7 aa, synthetic construct
RASNLES

SEQ ID NO: 588 - protein, 9 aa, synthetic construct
QQSNEDPWT

SEQ ID NO: 589 - protein, 13 aa, synthetic construct
KATGYTFSSSWIE

SEQ ID NO: 590 - protein, 10 aa, synthetic construct
EILPGSGSTN

SEQ ID NO: 591 - protein, 15 aa, synthetic construct
ARSGAGGYDSGAFTY

SEQ ID NO: 592 - protein, 15 aa, synthetic construct
RASESIDTYGNSFMH

SEQ ID NO: 593 - protein, 8 aa, synthetic construct
YRASNLES

SEQ ID NO: 594 - protein, 9 aa, synthetic construct
QQSNEDPWT

SEQ ID NO: 595 - protein, 8 aa, synthetic construct
GYTFSSSW

SEQ ID NO: 596 - protein, 8 aa, synthetic construct
ILPGSGST

SEQ ID NO: 597 - protein, 15 aa, synthetic construct
ARSGAGGYDSGAFTY

SEQ ID NO: 598 - protein, 10 aa, synthetic construct
ESIDTYGNSF

SEQ ID NO: 599 - skipped

SEQ ID NO: 600 - protein, 9 aa, synthetic construct
QQSNEDPWT

SEQ ID NO: 601 - protein, 8 aa, synthetic construct
GYTFSSSW

SEQ ID NO: 602 - protein, 8 aa, synthetic construct
ILPGSGTT

SEQ ID NO: 603 - protein, 15 aa, synthetic construct
ARSGAGGYDSGAFTY

SEQ ID NO: 604 - protein, 10 aa, synthetic construct
ESIDTYGNSF

SEQ ID NO: 605 - skipped

SEQ ID NO: 606 - protein, 9 aa, synthetic construct
QQSNEDPWT

SEQ ID NO: 607 - protein, 5 aa, synthetic construct
SSWIE

SEQ ID NO: 608 - protein, 17 aa, synthetic construct
EILPGSGTTNYNEKFKG

SEQ ID NO: 609 - protein, 13 aa, synthetic construct
SGAGGYDSGAFTY

SEQ ID NO: 610 - protein, 15 aa, synthetic construct
RASESIDTYGNSFMH

SEQ ID NO: 611 - protein, 7 aa, synthetic construct
RASNLES

SEQ ID NO: 612 - protein, 9 aa, synthetic construct
QQSNEDPWT

SEQ ID NO: 613 - protein, 7 aa, synthetic construct
GYTFSSS

SEQ ID NO: 614 - protein, 6 aa, synthetic construct
LPGSGT

SEQ ID NO: 615 - protein, 13 aa, synthetic construct
SGAGGYDSGAFTY

SEQ ID NO: 616 - protein, 15 aa, synthetic construct
RASESIDTYGNSFMH

SEQ ID NO: 617 - protein, 7 aa, synthetic construct
RASNLES

SEQ ID NO: 618 - protein, 9 aa, synthetic construct
QQSNEDPWT

SEQ ID NO: 619 - protein, 13 aa, synthetic construct
KATGYTFSSSWIE

SEQ ID NO: 620 - protein, 10 aa, synthetic construct
EILPGSGTTN

SEQ ID NO: 621 - protein, 15 aa, synthetic construct
ARSGAGGYDSGAFTY

SEQ ID NO: 622 - protein, 15 aa, synthetic construct
RASESIDTYGNSFMH

SEQ ID NO: 623 - protein, 8 aa, synthetic construct
YRASNLES

SEQ ID NO: 624 - protein, 9 aa, synthetic construct
QQSNEDPWT

SEQ ID NO: 625 - protein, 8 aa, synthetic construct
GYTFSSSW

SEQ ID NO: 626 - protein, 8 aa, synthetic construct
ILPGSGTT

SEQ ID NO: 627 - protein, 15 aa, synthetic construct
ARSGAGGYDSGAFTY

SEQ ID NO: 628 - protein, 10 aa, synthetic construct
ESIDTYGNSF

SEQ ID NO: 629 - skipped

SEQ ID NO: 630 - protein, 9 aa, synthetic construct
QQSNEDPWT

SEQ ID NO: 631 - protein, 8 aa, synthetic construct
GNTFSSYW

SEQ ID NO: 632 - protein, 8 aa, synthetic construct
ILPGRGST

SEQ ID NO: 633 - protein, 15 aa, synthetic construct
ARSGGGGYDSGAFAY

SEQ ID NO: 634 - protein, 10 aa, synthetic construct
ESVDTYGNSF

SEQ ID NO: 635 - skipped

SEQ ID NO: 636 - protein, 9 aa, synthetic construct
QQSNEDPWT

SEQ ID NO: 637 - protein, 5 aa, synthetic construct
SYWIE

SEQ ID NO: 638 - protein, 17 aa, synthetic construct
EILPGRGSTNNNEKFKG

SEQ ID NO: 639 - protein, 13 aa, synthetic construct
SGGGGYDSGAFAY

SEQ ID NO: 640 - protein, 15 aa, synthetic construct
RASESVDTYGNSFMH

SEQ ID NO: 641 - protein, 7 aa, synthetic construct
RTSNLES

SEQ ID NO: 642 - protein, 9 aa, synthetic construct
QQSNEDPWT

SEQ ID NO: 643 - protein, 7 aa, synthetic construct
GNTFSSY

SEQ ID NO: 644 - protein, 6 aa, synthetic construct
LPGRGS

SEQ ID NO: 645 - protein, 13 aa, synthetic construct
SGGGGYDSGAFAY

SEQ ID NO: 646 - protein, 15 aa, synthetic construct
RASESVDTYGNSFMH

SEQ ID NO: 647 - protein, 7 aa, synthetic construct
RTSNLES

SEQ ID NO: 648 - protein, 9 aa, synthetic construct
QQSNEDPWT

SEQ ID NO: 649 - protein, 13 aa, synthetic construct
KTTGNTFSSYWIE

SEQ ID NO: 650 - protein, 10 aa, synthetic construct
EILPGRGSTN

SEQ ID NO: 651 - protein, 15 aa, synthetic construct
ARSGGGGYDSGAFAY

SEQ ID NO: 652 - protein, 15 aa, synthetic construct
RASESVDTYGNSFMH

SEQ ID NO: 653 - protein, 8 aa, synthetic construct
YRTSNLES

SEQ ID NO: 654 - protein, 9 aa, synthetic construct
QQSNEDPWT

SEQ ID NO: 655 - protein, 8 aa, synthetic construct
GNTFSSYW

SEQ ID NO: 656 - protein, 8 aa, synthetic construct
ILPGRGST

SEQ ID NO: 657 - protein, 15 aa, synthetic construct
ARSGGGGYDSGAFAY

SEQ ID NO: 658 - protein, 10 aa, synthetic construct
ESVDTYGNSF

SEQ ID NO: 659 - skipped

SEQ ID NO: 660 - protein, 9 aa, synthetic construct
QQSNEDPWT

SEQ ID NO: 661 - protein, 8 aa, synthetic construct
GYTFSSSW

SEQ ID NO: 662 - protein, 8 aa, synthetic construct
ILPGSGNT

SEQ ID NO: 663 - protein, 14 aa, synthetic construct
ARSGLYYDYGGFAY

SEQ ID NO: 664 - protein, 10 aa, synthetic construct
ESVDTYGNSF

SEQ ID NO: 665 - skipped

SEQ ID NO: 666 - protein, 9 aa, synthetic construct
QQSNEDPFT

SEQ ID NO: 667 - protein, 5 aa, synthetic construct
SSWIE

SEQ ID NO: 668 - protein, 17 aa, synthetic construct
EILPGSGNTSYNEKFKG

SEQ ID NO: 669 - protein, 12 aa, synthetic construct
SGLYYDYGGFAY

SEQ ID NO: 670 - protein, 15 aa, synthetic construct
RASESVDTYGNSFMH

SEQ ID NO: 671 - protein, 7 aa, synthetic construct
RTSNLES

SEQ ID NO: 672 - protein, 9 aa, synthetic construct
QQSNEDPFT

SEQ ID NO: 673 - protein, 7 aa, synthetic construct
GYTFSSS

SEQ ID NO: 674 - protein, 6 aa, synthetic construct
LPGSGN

SEQ ID NO: 675 - protein, 12 aa, synthetic construct
SGLYYDYGGFAY

SEQ ID NO: 676 - protein, 15 aa, synthetic construct
RASESVDTYGNSFMH

SEQ ID NO: 677 - protein, 7 aa, synthetic construct
RTSNLES

SEQ ID NO: 678 - protein, 9 aa, synthetic construct
QQSNEDPFT

SEQ ID NO: 679 - protein, 13 aa, synthetic construct
KATGYTFSSSWIE

SEQ ID NO: 680 - protein, 10 aa, synthetic construct
EILPGSGNTS

SEQ ID NO: 681 - protein, 14 aa, synthetic construct
ARSGLYYDYGGFAY

SEQ ID NO: 682 - protein, 15 aa, synthetic construct
RASESVDTYGNSFMH

SEQ ID NO: 683 - protein, 8 aa, synthetic construct
YRTSNLES

SEQ ID NO: 684 - protein, 9 aa, synthetic construct
QQSNEDPFT

SEQ ID NO: 685 - protein, 8 aa, synthetic construct
GYTFSSSW

SEQ ID NO: 686 - protein, 8 aa, synthetic construct
ILPGSGNT

SEQ ID NO: 687 - protein, 14 aa, synthetic construct
ARSGLYYDYGGFAY

SEQ ID NO: 688 - protein, 10 aa, synthetic construct
ESVDTYGNSF

SEQ ID NO: 689 - skipped

SEQ ID NO: 690 - protein, 9 aa, synthetic construct
QQSNEDPFT

SEQ ID NO: 691 - protein, 8 aa, synthetic construct
GFNIKDYY

SEQ ID NO: 692 - protein, 8 aa, synthetic construct
IDPENGNT

SEQ ID NO: 693 - protein, 11 aa, synthetic construct
ARSRDHGAMDY

SEQ ID NO: 694 - protein, 7 aa, synthetic construct
SSVSSSN

SEQ ID NO: 695 - skipped

SEQ ID NO: 696 - protein, 9 aa, synthetic construct
HQWSSYPYT

SEQ ID NO: 697 - protein, 5 aa, synthetic construct
DYYMH

SEQ ID NO: 698 - protein, 17 aa, synthetic construct
WIDPENGNTVYAPKFQG

SEQ ID NO: 699 - protein, 9 aa, synthetic construct
SRDHGAMDY

SEQ ID NO: 700 - protein, 12 aa, synthetic construct
SASSSVSSSNLY

SEQ ID NO: 701 - protein, 7 aa, synthetic construct
STSNLAS

SEQ ID NO: 702 - protein, 9 aa, synthetic construct
HQWSSYPYT

SEQ ID NO: 703 - protein, 7 aa, synthetic construct
GFNIKDY

SEQ ID NO: 704 - protein, 6 aa, synthetic construct
DPENGN

SEQ ID NO: 705 - protein, 9 aa, synthetic construct
SRDHGAMDY

SEQ ID NO: 706 - protein, 12 aa, synthetic construct
SASSSVSSSNLY

SEQ ID NO: 707 - protein, 7 aa, synthetic construct
STSNLAS

SEQ ID NO: 708 - protein, 9 aa, synthetic construct
HQWSSYPYT

SEQ ID NO: 709 - protein, 13 aa, synthetic construct
KGSGFNIKDYYMH

SEQ ID NO: 710 - protein, 10 aa, synthetic construct
WIDPENGNTV

SEQ ID NO: 711 - protein, 11 aa, synthetic construct
ARSRDHGAMDY

SEQ ID NO: 712 - protein, 12 aa, synthetic construct
SASSSVSSSNLY

SEQ ID NO: 713 - protein, 8 aa, synthetic construct
YSTSNLAS

SEQ ID NO: 714 - protein, 9 aa, synthetic construct
HQWSSYPYT

SEQ ID NO: 715 - protein, 8 aa, synthetic construct
GFNIKDYY

SEQ ID NO: 716 - protein, 8 aa, synthetic construct
IDPENGNT

SEQ ID NO: 717 - protein, 11 aa, synthetic construct
ARSRDHGAMDY

SEQ ID NO: 718 - protein, 7 aa, synthetic construct
SSVSSSN

SEQ ID NO: 719 - skipped

SEQ ID NO: 720 - protein, 9 aa, synthetic construct
HQWSSYPYT

SEQ ID NO: 721 - protein, 8 aa, synthetic construct
GYTFTDYS

SEQ ID NO: 722 - protein, 8 aa, synthetic construct
INTETGEP

SEQ ID NO: 723 - protein, 11 aa, synthetic construct
ARVVRRGYFDV

SEQ ID NO: 724 - protein, 6 aa, synthetic construct
GNIHNY

SEQ ID NO: 725 - skipped

SEQ ID NO: 726 - protein, 9 aa, synthetic construct
QHFWSTPFT

SEQ ID NO: 727 - protein, 5 aa, synthetic construct
DYSMH

SEQ ID NO: 728 - protein, 17 aa, synthetic construct
WINTETGEPTYADDFKG

SEQ ID NO: 729 - protein, 9 aa, synthetic construct
VVRRGYFDV

SEQ ID NO: 730 - protein, 11 aa, synthetic construct
RASGNIHNYLA

SEQ ID NO: 731 - protein, 7 aa, synthetic construct
NAKTLAD

SEQ ID NO: 732 - protein, 9 aa, synthetic construct
QHFWSTPFT

SEQ ID NO: 733 - protein, 7 aa, synthetic construct
GYTFTDY

SEQ ID NO: 734 - protein, 6 aa, synthetic construct
NTETGE

SEQ ID NO: 735 - protein, 9 aa, synthetic construct
VVRRGYFDV

SEQ ID NO: 736 - protein, 11 aa, synthetic construct
RASGNIHNYLA

SEQ ID NO: 737 - protein, 7 aa, synthetic construct
NAKTLAD

SEQ ID NO: 738 - protein, 9 aa, synthetic construct
QHFWSTPFT

SEQ ID NO: 739 - protein, 13 aa, synthetic construct
KASGYTFTDYSMH

SEQ ID NO: 740 - protein, 10 aa, synthetic construct
WINTETGEPT

SEQ ID NO: 741 - protein, 11 aa, synthetic construct
ARVVRRGYFDV

SEQ ID NO: 742 - protein, 11 aa, synthetic construct
RASGNIHNYLA

SEQ ID NO: 743 - protein, 8 aa, synthetic construct
YNAKTLAD

SEQ ID NO: 744 - protein, 9 aa, synthetic construct
QHFWSTPFT

SEQ ID NO: 745 - protein, 8 aa, synthetic construct
GYTFTDYS

SEQ ID NO: 746 - protein, 8 aa, synthetic construct
INTETGEP

SEQ ID NO: 747 - protein, 11 aa, synthetic construct
ARVVRRGYFDV

SEQ ID NO: 748 - protein, 6 aa, synthetic construct
GNIHNY

SEQ ID NO: 749 - skipped

SEQ ID NO: 750 - protein, 9 aa, synthetic construct
QHFWSTPFT

SEQ ID NO: 751 - protein, 127 aa, synthetic construct
EVQLQQSGPELVKPGASVKMSCKASGYTFTRYVMHWVKQKPGQGLEWIGYINPNNDGTKYNEKFKGKVTLTSDKSSSTAYMELSSLTSEDSAVYYCARGDLEILPYYDHARNAMDYWGQGTSVTVSS

SEQ ID NO: 752 - protein, 107 aa, synthetic construct
DIVMTQSPASQSASLGESVIITCRASGNIHNYLAWYQQKQGKSPQLLVYNAKTLADGVPSRFSGSGSGTQYSLKINSLQPEDFGTYYCQHFWSSPPTFGGGTKLELK

SEQ ID NO: 753 - protein, 127 aa, synthetic construct; identical to SEQ ID NO: 751

SEQ ID NO: 754 - protein, 107 aa, synthetic construct
DVQMIQSPASLSASVGETVTITCRASENIYSYLAWYQQKQGKSPQLLVYNAKTLAEGVPSRFSGSGSGTQFSLKINSLQPEDFGSYYCQHFWSTPPTFGGGTKLEIK

SEQ ID NO: 755 - protein, 127 aa, synthetic construct
QIQLLQSGPELVKPGASVKMSCKASGYTFTRYVMYWVKQKPGQGLEWIGYINPDNDGTKYNEKFKGKATLTSDKSSSTAYMELSSLTSEDSAVYYCARGDLELLPYYDYVRNAMDYWGQGTSVTVSS

SEQ ID NO: 756 - protein, 107 aa, synthetic construct
DIQMTQSPASLSASVGETVTITCRASGNIHNYLAWYQQKQGKSPQLLVYNAKTLADGVPSRFSGSGSGTQYSLKINSLQPEDFGSYYCQHFWSTPPTFGGGTKLEIK

SEQ ID NO: 757 - protein, 120 aa, synthetic construct
EVQLQQSGAELVRPGALVKLSCKASGFNIKDYYMHWVKQRPEQGLEWIGWIDPESGNTIYDPKFQGKASMTADTSSNTAYLQLSSLTSEDTAVYYCARLDGSSYYAMDYWGQGTSVTVSS

SEQ ID NO: 758 - protein, 111 aa, synthetic construct
DILLTQSPASLAVSLGQRATISCRASESVDSYGNSFMHWYQQKPGQPPKLLIYLASNLESGVPARFSGSGSRTDFTLTIDPVEADDAATYYCQQNNEDPLTFGAGTKLELK

SEQ ID NO: 759 - protein, 122 aa, synthetic construct
EVQLQQPGAELMKPGASVKISCKATGYTFSSSWTEWVKQRPGHGLEWIGEILPGSGSTNYNEKFKGKATFTADTSSNTAYMQLSSLTSEDSAVYYCARSGAGGYDSGAFAYWGQGTLVTVSA

SEQ ID NO: 760 - protein, 111 aa, synthetic construct
DIVLTQSPASLAVSLGQRATISCRASESIDTYGNSFMHWYQQKPGQPPKLLIYRTSNLESGIPARFSGSGSRTDFTLTINPVEADDVATYYCQQSNEDPWTFGGGTKLEIK

SEQ ID NO: 761 - protein, 122 aa, synthetic construct
EVQLQQSGAELMKPGASVKISCKATGYTFSNYWIEWVKQRPGHGLEWIGEILPGSGSTDYNEKFEGKATFTADTSSNTAYMQLSSLTSEDSAVYYCARSGAGGYDSGAFAYWGQGTLVTVSA

SEQ ID NO: 762 - protein, 111 aa, synthetic construct
DIQMTQSPASLAVSLGQRATISCRASESVDTYGNSFMHWYQQKPGQPPKLLIYRTSNLESGIPARFSGSGSRTDFTLTINPVEADDVATYYCQQSNEDPWTFGGGTKLEIK

SEQ ID NO: 763 - protein, 122 aa, synthetic construct
QIQLLQSGAELMKPGASVKISCKATGYTFSNYWIEWVKQRPGHGLEWIGEILPGSGSTNYNEKFKGKATFTADTSSNTAYMQLSSLTSEDSAVYYCARSGAGGYDSGAFAYWGQGTLVTVSS

SEQ ID NO: 764 - protein, 111 aa, synthetic construct
DIVLTQSPASLAVSLGQRATISCRASESVDSYGNSFMHWYQQKPGQPPKLLIYRTSNLESGIPARFSGSGSRTDFTLTINPVEADDVAAYYCQQSNEDPWTFGGGTKLEIK

SEQ ID NO: 765 - protein, 122 aa, synthetic construct
EVQLQQSGAELMKPGASVKISCKATGNTFSSYWIEWVKQRPGHDLEWIGEILPGSGSTNYNEKFKGKATFTADTSSNTAYMQLNSLTSEDSAVYYCARSGAGGYDSGAFAYWGQGTLVTVSA

SEQ ID NO: 766 - protein, 111 aa, synthetic construct
DIEMTQSPASLAVSLGQRATISCRASESIDSYGNSFMHWYQQKPGQPPKLLIYRTSNLESGIPARFSGSGSRTDFTLTINPVEADDVATYYCQQSNEDPWTFGGGTKLEIK

SEQ ID NO: 767 - protein, 122 aa, synthetic construct
EVQLQQSGAELMKPGASVKISCKTTGNTFSSYWIEWVKQRPGHDLEWIGEILPGSGSSNYNEKFKGKATFTADTSSNTAYMQLNSLTSEDSAVYYCARSGAGGYDSGAFAYWGQGTLVTVSA

SEQ ID NO: 768 - protein, 111 aa, synthetic construct
DIVLTQSPASLAVSLGQRATISCRASESVDSYGNSFMHWYQQKPGQPPKLLIYRTSNLESGIPSRFSGSGSRTDFTLTINPVEADDVATYYCQQSNEDPWTFGGGTKLEIK

SEQ ID NO: 769 - protein, 122 aa, synthetic construct
EVQLQQSGAELMKPGASVKISCKTTGNTFSSYWIEWVKQRPGHDLEWIGEILPGSGSTNYNEKFKGKATFTADTSSNTAYMQLNSLTSEDSAVYYCARSGAGGYDSGAFAYWGQGTLVTVSA

SEQ ID NO: 770 - protein, 111 aa, synthetic construct
DIVLTQSPASLAVSLGQRATISCRASESVDSYGNSFMHWYQQKPGQPPKLLIYRTSNLESGIPARFSGSGSRTDFTLTINPVEADDVATYYCQQSNEDPWTFGGGTKLEIK

SEQ ID NO: 771 - protein, 122 aa, synthetic construct
QVQFQQSGVELMKPGASVKISCKATGNTFSNYWIEWVKQRPGHDLEWIGEILPGSGSTNYNEKFKGKATFTADTSSNTAYMQLNSLTSEDSAVYYCARSGAGGYDSGAFAYWGQGTLVTVSA

SEQ ID NO: 772 - protein, 111 aa, synthetic construct
DIVMTQSPASLAVSLGQRATISCRASESVDSYGNSFMHWYQQKPGQPPKLLIYRTSNLESGIPARFSGSGSRTDFTLTINPVEADDVATYYCQQSNEDPWTFGGGTKLEIK

SEQ ID NO: 773 - protein, 122 aa, synthetic construct
QVQLQQSGAELMKPGASVKISCKATGSTFSSYWIEWVKQRPGHGLEWIGEILPGSGSTNYNEKFKGKATFTADTSSNTAYMQLSSLTSEDSAVYYCARSGAGGYDSGAFAYWGQGTLVTVSA

SEQ ID NO: 774 - protein, 111 aa, synthetic construct
DIVLTQSPASLAVSLGQRATISCRASESVDTYGNSFMHWYQQKPGQPPKLLIYRTSNLESGIPARFSGSGSRTDFTLTINPVEADDVATYYCQQSNEDPWTFGGGTKLEIK

SEQ ID NO: 775 - protein, 122 aa, synthetic construct
QVQLQQSGAELMKPGASVKISCKATGYTLSSYWIEWVKQRPGHGLEWIGEILPGSGSTNYNEKFKGKATFTADTSSNTAYMQLSSLTSEDSAVYYCARSGAGGYDSGAFAYWGQGTLVTVSA

SEQ ID NO: 776 - protein, 111 aa, synthetic construct; identical to SEQ ID NO: 774

SEQ ID NO: 777 - protein, 122 aa, synthetic construct
QVQMQQPGAELMKPGASVKISCKATGYTFSRYWIEWVKQRPGHGLEWIGEILPGDGSTNYNAKFKGQATFTADTSSNTAYMQFSSLTYEDSAVYYCARSGAGGYDSGAFAYWGQGTLVTVSS

SEQ ID NO: 778 - protein, 111 aa, synthetic construct; identical to SEQ ID NO: 774

SEQ ID NO: 779 - protein, 122 aa, synthetic construct
QVQLQQPGAELMKPGASVKISCKATGYTFSSYWIEWVKQRPGHGLEWIGEILPGDGSTNYNAKFKGQATFTADTSSNTAYMQFSSLTSEDSAVYYCARSGAGGYDSGAFAYWGQGTLVTVSS

SEQ ID NO: 780 - protein, 111 aa, synthetic construct
DIVLTQSPASLAVSLGQRATISCRASESVDTYGNSFMHWYQQKPGQPPKLLVYRTSNLESGIPARFSGSGSGTDFTLTINPVEADDVATYYCQQSNEDPWTFGGGTKLEIK

SEQ ID NO: 781 - protein, 122 aa, synthetic construct
EVQLQQSGAELMKPGASVKISCKITGYTFSSYWIEWVKQRPGHDLEWIGEILPGSGSTNYNEKFKGKASFTADTSSNTAYMQLNSLTSEDSAVYYCARSGAGGYDSGAFAYWGQGTLVTVSA

SEQ ID NO: 782 - protein, 111 aa, synthetic construct; identical to SEQ ID NO: 774

SEQ ID NO: 783 - protein, 122 aa, synthetic construct
EVQLQQSGAELMKPGASVKISCKATGYTFSSYWIEWVKQRPGHGLEWIGEILPGSGSTNYNEKFKGKATFTADTSSNTAYMQLSSLTSEDSAVYYCARSGAGGYDSGAFAYWGQGTLVTVSS

SEQ ID NO: 784 - protein, 111 aa, synthetic construct
DIVMTQSPASLAVSLGQRATISCRASESVDTYGNSFMHWYQQKPGQPPKLLIYRTSNLESGIPARFSGSGSRTDFTLTINPVEADDVATYYCQQSNEDPWTFGGGTKLEIK

SEQ ID NO: 785 - protein, 122 aa, synthetic construct
EVQLQQSGAEVKKPGASVKISCKATGYTFSSYWIEWVKQRPGHGLEWIGEILPGSGSTNYNEKFKGKATFTADTSSNTAYMQLSSLTSEDFTVYYCARSGAGGYDSGAFGYWGQGTLVTVSS

SEQ ID NO: 786 - protein, 111 aa, synthetic construct
DIVMTQSPASLAVSLGQRATISCRASESIDTYGNSFMHWYQQKPGQPPKLLIYRASNLESGIPARFSGSGSRTDFTLTINPVEADDVATYYCQQSNEDPWTFGGGTKLEIK

SEQ ID NO: 787 - protein, 122 aa, synthetic construct
QVQMQPFGAELMKPGASVRISCKATGYTFSSSWIEWVKQRPGHGLEWIGEILPGSGTTNYNEKFKGKATFTADTSSNTAYMQLSSLTSEDSAVYYCARSGAGGYDSGAFTYWGQGTLVTVSA

SEQ ID NO: 788 - protein, 111 aa, synthetic construct
DIVLTQSPASLAVSLGQRATISCRASESIDTYGNSFMHWYQQKPGQPPKLLIYRASNLESGIPARFSGSGSRTDFTLTINPVEADDVATYYCQQSNEDPWTFGGGTKLEIK

SEQ ID NO: 789 - protein, 122 aa, synthetic construct
QVQLQQSGAELMKPGASVRISCKATGYTFSSSWIEWVKQRPGHGLEWIGEILPGSGSTNYNEKFKGKATFTADTSSNTAYMQLSSLTSEDSAVYYCARSGAGGYDSGAFTYWGQGTLVTVSA

SEQ ID NO: 790 - protein, 111 aa, synthetic construct; identical to SEQ ID NO: 788

SEQ ID NO: 791 - protein, 122 aa, synthetic construct
QVQLQQSGAELMKPGASVRISCKATGYTFSSSWIEWVKQRPGHGLEWIGEILPGSGTTNYNEKFKGKATFTADTSSNTAYMQLSSLTSEDSAVYYCARSGAGGYDSGAFTYWGQGTLVTVSA

SEQ ID NO: 792 - protein, 111 aa, synthetic construct; identical to SEQ ID NO: 788

SEQ ID NO: 793 - protein, 122 aa, synthetic construct
EVQLQQSGAELMKPGASVKISCKTTGNTFSSYWIEWVKQRPGHDLEWIGEILPGRGSTNNNEKFKGKAPFTADTSSNTAYMQLNSLTSEDSAVYYCARSGGGGYDSGAFAYWGQGTLVTVSA

SEQ ID NO: 794 - protein, 111 aa, synthetic construct; identical to SEQ ID NO: 774

SEQ ID NO: 795 - protein, 121 aa, synthetic construct
DVQLQQSGAELMKPGASVKISCKATGYTFSSSWIEWVKQRPGHGLEWIGEILPGSGNTSYNEKFKGKATFTADTSSSAAYMQLSSLTSEDSAVYYCARSGLYYDYGGFAYWGQGTLVTVSA

SEQ ID NO: 796 - protein, 111 aa, synthetic construct
DIVLTQSPASLAVSLGQRATISCRASESVDTYGNSFMHWYQQKPGQPPKLLIYRTSNLESGIPARFSGSGSRTDFTLTINPVEADDVATYYCQQSNEDPFTFGSGTKLEIK

SEQ ID NO: 797 - protein, 118 aa, synthetic construct
EVQLQQSGAELVRPGALVKLSCKGSGFNIKDYYMHWVRQRPEQGLEWIGWIDPENGNTVYAPKFQGKASITSDTSSNIAYLQFSSLTSEDTAVYYCARSRDHGAMDYWGQGTSVTVSS

SEQ ID NO: 798 - protein, 108 aa, synthetic construct
DVQITQSPAIMSASPGEKVTLTCSASSSVSSSNLYWYQQKPGSSPKLWIYSTSNLASGVPARFSGSGFGTSYALTISSMEAEDAASYFCHQWSSYPYTFGGGTKLEIK

SEQ ID NO: 799 - protein, 118 aa, synthetic construct
QIQLLQSGPELKKPGETVKISCKASGYTFTDYSMHWVKQAPGKGLKWMGWINTETGEPTYADDFKGRFAFSLETSASTAYLQINNLKNEDTATYFCARVVRRGYFDVWGAGTTVTVSS

SEQ ID NO: 800 - protein, 107 aa, synthetic construct
DIQMTQSPASLSASVGETVTITCRASGNIHNYLAWYQQKQGKSPQVLVYNAKTLADGVPSRFSGSGSGTQYSLKINSLQPEDFGSYYCQHFWSTPFTFGSGTKLEIK

SEQ ID NO: 801 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 802 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 803 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 804 - protein, 110 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAK

SEQ ID NO: 805 - protein, 107 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 806 - protein, 107 aa, synthetic construct [sequence omitted]

SEQ ID NO: 807 - protein, 457 aa, synthetic construct
EVQLQQSGPELVKPGASVKMSCKASGYTFTRYVMHWVKQKPGQGLEWIGYINPNNDGTKYNEKFKGKVTLTSDKSSSTAYMELSSLTSEDSAVYYCARGDLEILPYYDHARNAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 808 - protein, 214 aa, synthetic construct
DVQMIQSPASLSASVGETVTITCRASENIYSYLAWYQQKQGKSPQLLVYNAKTLAEGVPSRFSGSGSGTQFSLKINSLQPEDFGSYYCQHFWSTPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 809 - protein, 452 aa, synthetic construct
QVQLQQPGAELMKPGASVKISCKATGYTFSSYWIEWVKQRPGHGLEWIGEILPGDGSTNYNAKFKGQATFTADTSSNTAYMQFSSLTSEDSAVYYCARSGAGGYDSGAFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 810 - protein, 218 aa, synthetic construct
DIVLTQSPASLAVSLGQRATISCRASESVDTYGNSFMHWYQQKPGQPPKLLVYRTSNLESGIPARFSGSGSGTDFTLTINPVEADDVATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 811 - protein, 451 aa, synthetic construct
EVQLQQSGPELVKPGASVKMSCKASGYTFTRYVMHWVKQKPGQGLEWIGYINPNNDGTKYNEKFKGKVTLTSDKSSSTAYMELSSLTSEDSAVYYCARGDLEILPYYDHARNAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 812 - protein, 214 aa, synthetic construct
DVQMIQSPASLSASVGETVTITCRASENIYSYLAWYQQKQGKSPQLLVYNAKTLAEGVPSRFSGSGSGTQFSLKINSLQPEDFGSYYCQHFWSTPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 813 - protein, 651 aa, synthetic construct [sequence omitted]

SEQ ID NO: 814 - protein, 598 aa, synthetic construct
MTPIVTVLICLGLSLGPRTHVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEDEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAYNLSSEWSAPSDPLDILITGQIHGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEGEPPAEPSIYATLAIH

SEQ ID NO: 815 - protein, 459 aa, synthetic construct
GHLPKPTLWAEPGSVITQGSPVTLRCQGGQETQEYRLYREKKTAPWITRIPQELVKKGQFPIPSITWEHAGRYRCYYGSDTAGRSESSDPLELVVTGAYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVHHHHHHGLNDIFEAQKIEWHE

SEQ ID NO: 816 - protein, 598 aa, Macaca fascicularis
MTPILMVLICLGLSLGPRTHVQAGILPKPTLWAEPGSVISEGSPVTLRCQGSLQVQEYHLYREKNPASWVRQIRQELVKKGYFAIGFITWEHTGQYRCQYYSHSWWSEPSDPLELVVTGAYSKPTLSALPSPVVASGGNVTLQCDSQVAFDSFTLCKEGEDEHPQRLNCQSHARGWSWAVFSVGPVSPSRRWSYRCYGYISSAPNVWSLPSDLLELLVPGVSKKPSLSVQPGPVVAPGDKLTLQCGSDAGYDRFALYKEGEGDFLQRPVRQPQAGLSQANFLLGPVSRSHGGQYRCSGAHNLSSEWSAPSDPLDILIAGQIRGRPFLSVQPGPKVVSGENVTLLCQSSWQFHAFLLTQAGAADAHLHLRSMYKYPKYQAEFPMSPVTSAHAGTYRCYGSRSSNPYLLSVPSDPLELVVSGPSGGPSSPTTGPTSTCAGPEDQPLTPTGSAPQSGLGRHLGVVTGVLVAFVLLLFLLLLLFLVLRYRRQGKRWTSAQRKADFQHPAGAVEPEPRDRGLQRRSSPAADTQEENLYAAVKDTQPEDGVELDSRAAASEDPQDVTYAQLQSLTLRREATEPPPSQERAPPVESSIYATLTIH

SEQ ID NO: 817 - protein, 439 aa, Macaca fascicularis
MGWSCIILFLVATATGVHSQAGILPKPTLWAEPGSVISEGSPVTLRCQGSLQVQEYHLYREKNPASWVRQIRQELVKKGYFAIGFITWEHTGQYRCQYYSHSWWSEPSDPLELVVTGAYSKPTLSALPSPVVASGGNVTLQCDSQVAFDSFTLCKEGEDEHPQRLNCQSHARGWSWAVFSVGPVSPSRRWSYRCYGYISSAPNVWSLPSDLLELLVPGVSKKPSLSVQPGPVVAPGDKLTLQCGSDAGYDRFALYKEGEGDFLQRPVRQPQAGLSQANFLLGPVSRSHGGQYRCSGAHNLSSEWSAPSDPLDILIAGQIRGRPFLSVQPGPKVVSGENVTLLCQSSWQFHAFLLTQAGAADAHLHLRSMYKYPKYQAEFPMSPVTSAHAGTYRCYGSRSSNPYLLSVPSDPLELVVSGHHHHHHGLNDIFEAQKIEWHE

SEQ ID NO: 818 - protein, 390 aa, Macaca fascicularis
MGWSCIILFLVATATGVHSQAGILPKPMLWAEPDRVITQGSPVTLRCQGNLEARGYHLYRERKSASWITLIRPELVKKGQFPIPSITWEDAGRYRCQYYSHSWWSEHSDPLELVVTGAYRKPTLSALPSPVVASGGNVTLQCDSRVALDGFILCKEGEDEHSQRLNSQPRTRGSSRAVFSVGPVSPSRRWSYRCYGYELHSRYVWSLPSDLLELLVPGVSKKPSLSVQPGPVVAGGDKLTLQCGSDAGYDRFALYKEGERDFLQRPGQQLQAGLAQANFTLDPVRGSHGGQYRCYGAHNLSSEWSAPSDPLDILISAGPHSGLRRECDPAVSVTGMDGHFLSDQGGSSSPGGGSGGGSEQKLISEEDLGHHHHHHGLNDIFEAQKIEWHE

SEQ ID NO: 819 - protein, 598 aa, Macaca fascicularis
MTPIVTVLICLGLSLGPRTHVQAGILPKPMLWAEPDRVITQGSPVTLRCQGNLEARGYHLYRERKSASWITLIRPELVKKGQFPIPSITWEDAGRYRCQYYSHSWWSEHSDPLELVVTGAYRKPTLSALPSPVVASGGNVTLQCDSRVALDGFILCKEGEDEHSQRLNSQPRTRGSSRAVFSVGPVSPSRRWSYRCYGYELHSRYVWSLPSDLLELLVPGVSKKPSLSVQPGPVVAGGDKLTLQCGSDAGYDRFALYKEGERDFLQRPGQQLQAGLAQANFTLDPVRGSHGGQYRCYGAYNLSSEWSAPSDPLDILITGQIHGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEGEPPAEPSIYATLAIH

SEQ ID NO: 820 - protein, 693 aa, synthetic construct
MTPIVTVLICLGLSLGPRTHVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEDEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAYNLSSEWSAPSDPLDILITGQIHGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 821 - protein, 693 aa, synthetic construct
MTPILTVLICLGLSLGPRTHVQAGHLPKPTLWAEPGSVITQGSPVTLRCQGGQETQEYRLYREKKTAPWITRIPQELVKKGQFPIPSITWEHAGRYRCYYGSDTAGRSESSDPLELVVTGAYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 822 - protein, 638 aa, Macaca mulatta
MTPILTILICLGLSLGSRTRVQEGILPKPTLWAEPGSMISEGSPVTLRCQGSLQVQDYCLYREKKPASWVRQIRQELVKKGYFAIGFITWEHTGQYRCQYYNHSWWSEPSDPLELVVIGAYSKPTLSALPSPVVASGRNVTLQCDSQVAFDGFILCKEGEDEHPQRLNCQSHARGWSWAVFSVGPVSPSHRWSYRCYGYISSDPYVWSLPSDLLELLVPGVSKKPSLSVQPGPVVARGDKLTLQCGSDAGYDRFALYKEGEGDFLQRPGQQPQAGLSQANFLLGPVSRSHGGQYRCSGAHNLSPEWSAPSDPLDILISRQIRGRPFLSVQPGPKVVSGENVTLLCQSSWQFHAFLLTQAGAADAHLHLRSMHKYPKYQAEFPISPVTSAHAGTYRCYGSRSSNPYLLSVPSDPLELVVSGPSGGPSSPTTGPTSTCGPEDQPLTPTGSAPQSGLGRHLGVVTGVLVAFVLLLFLLLLFLVLRYRRQGKRWTSAQRKADFQHPAGAVEPEPRDRGLQRRSSPAADTQEENLYAAVKDTQPEDGVELDSQRPHDEDPQAVTYARVKHSRPRREMASPPSPLSEEFLDTKDTQAAASEDPQDVTYAQLQSLTLRREATEPPPSQERAPPVESSIYATLTIH

SEQ ID NO: 823 - protein, 463 aa, synthetic construct
MGWSCIILFLVATATGVHSGHLPKPTLWAEPGSVITQGSPVTLRCQGGQETQEYRLYREKKTAPWITRIPQELVKKGQFPIPSITWEHAGRYRCYYGSDTAGRSESSDPLELVVTGAYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVLGGGGSGGGGSGGGGSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 824 - protein, 462 aa, synthetic construct
MGWSCIILFLVATATGVHSGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEDEHPQCLNSQPHARGSSRAIFSVGPVSPNRRWSHRCYGYDLNSPYVWSSPSDLLELLVPGGGGSGGGGSGGGGSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 825 - protein, 651 aa, synthetic construct [sequence omitted]

SEQ ID NO: 826 - protein, 598 aa, synthetic construct; identical to SEQ ID NO: 814

SEQ ID NO: 827 - protein, 631 aa, Homo sapiens
MTPALTALLCLGLSLGPRTRVQAGPFPKPTLWAEPGSVISWGSPVTIWCQGSQEAQEYRLHKEGSPEPLDRNNPLEPKNKARFSIPSMTEHHAGRYRCHYYSSAGWSEPSDPLEMVMTGAYSKPTLSALPSPVVASGGNMTLRCGSQKGYHHFVLMKEGEHQLPRTLDSQQLHSRGFQALFPVGPVTPSHRWRFTCYYYYTNTPWVWSHPSDPLEILPSGVSRKPSLLTLQGPVLAPGQSLTLQCGSDVGYNRFVLYKEGERDFLQRPGQQPQAGLSQANFTLGPVSPSNGGQYRCYGAHNLSSEWSAPSDPLNILMAGQIYDTVSLSAQPGPTVASGENVTLLCQSWWQFDTFLLTKEGAAHPPLRLRSMYGAHKYQAEFPMSPVTSAHAGTYRCYGSYSSNPHLLSHPSEPLELVVSGHSGGSSLPPTGPPSTPGLGRYLEVLIGVSVAFVLLLFLLLFLLLRRQRHSKHRTSDQRKTDFQRPAGAAETEPKDRGLLRRSSPAADVQEENLYAAVKDTQSEDRVELDSQSPHDEDPQAVTYAPVKHSSPRREMASPPSSLSGEFLDTKDRQVEEDRQMDTEAAASEASQDVTYAQLHSLTLRRKATEPPPSQEGEPPAEPSIYATLAIH

SEQ ID NO: 828 - protein, 631 aa, Homo sapiens
MTPALTALLCLGLSLGPRTRVQAGPFPKPTLWAEPGSVISWGSPVTIWCQGSLEAQEYRLDKEGSPEPLDRNNPLEPKNKARFSIPSMTEHHAGRYRCHYYSSAGWSEPSDPLELVMTGFYNKPTLSALPSPVVASGGNMTLRCGSQKGYHHFVLMKEGEHQLPRTLDSQQLHSGGFQALFPVGPVNPSHRWRFTCYYYYMNTPQVWSHPSDPLEILPSGVSRKPSLLTLQGPVLAPGQSLTLQCGSDVGYDRFVLYKEGERDFLQRPGQQPQAGLSQANFTLGPVSPSHGGQYRCYGAHNLSSEWSAPSDPLNILMAGQIYDTVSLSAQPGPTVASGENVTLLCQSWWQFDTFLLTKEGAAHPPLRLRSMYGAHKYQAEFPMSPVTSAHAGTYRCYGSYSSNPHLLSFPSEPLELMVSGHSGGSSLPPTGPPSTPGLGRYLEVLIGVSVAFVLLLFLLLFLLLRRQRHSKHRTSDQRKTDFQRPAGAAETEPKDRGLLRRSSPAADVQEENLYAAVKDTQSEDRVELDSQSPHDEDPQAVTYAPVKHSSPRREMASPPSSLSGEFLDTKDRQVEEDRQMDTEAAASEASQDVTYAQLHSLTLRRKATEPPPSQEGEPPAEPSIYATLAIH

SEQ ID NO: 829 - protein, 489 aa, Homo sapiens
MTPIVTVLICLRLSLGPRTHVQAGTLPKPTLWAEPGSVITQGSPVTLWCQGILETQEYRLYREKKTAPWITRIPQEIVKKGQFPIPSITWEHTGRYRCFYGSHTAGWSEPSDPLELVVTGAYIKPTLSALPSPVVTSGGNVTLHCVSQVAFGSFILCKEGEDEHPQCLNSQPRTHGWSRAIFSVGPVSPSRRWSYRCYAYDSNSPHVWSLPSDLLELLVLGVSKKPSLSVQPGPIVAPGESLTLQCVSDVSYDRFVLYKEGERDFLQLPGPQPQAGLSQANFTLGPVSRSYGGQYRCSGAYNLSSEWSAPSDPLDILIAGQFRGRPFISVHPGPTVASGENVTLLCQSWGPFHTFLLTKAGAADAPLRLRSIHEYPKYQAEFPMSPVTSAHSGTYRCYGSLSSNPYLLSHPSDSLELMVSGAAETLSPPQNKSDSKAGAANTLSPSQNKTASHPQDYTVENLIRMGIAGLVLVVLGILLFEAQHSQRSL

SEQ ID NO: 830 - protein, 481 aa, Homo sapiens
MTPALTALLCLGLSLGPRTRVQAGPFPKPTLWAEPGSVISWGSPVTIWCQGSLEAQEYQLDKEGSPEPLDRNNPLEPKNKARFSIPSMTQHHAGRYRCHYYSSAGWSEPSDPLELVMTGFYNKPTLSALPSPVVASGGNMTLRCGSQKGYHHFVLMKEGEHQLPRTLDSQQLHSGGFQALFPVGPVTPSHRWRFTCYYYYTNTPRVWSHPSDPLEILPSGVSRKPSLLTLQGPVLAPGQSLTLQCGSDVGYDRFVLYKEGERDFLQRPGQQPQAGLSQANFTLGPVSPSHGGQYRCYGAHNLSSEWSAPSDPLNILMAGQIYDTVSLSAQPGPTVASGENVTLLCQSRGYFDTFLLTKEGAAHPPLRLRSMYGAHKYQAEFPMSPVTSAHAGTYRCYGSYSSNPHLLSFPSEPLELMVSGHSGGSSLPPTGPPSTPASHAKDYTVENLIRMGMAGLVLVFLGILLFEAQHSQRNPQDAAGR

SEQ ID NO: 831 - protein, 338 aa, synthetic construct
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 832 - protein, 365 aa, synthetic construct [sequence omitted]

SEQ ID NO: 833 - protein, 362 aa, synthetic construct
MLVMAPRTVLLLLSAALALTETWAGSHSMRYFDTAMSRPGRGEPRFISVGYVDDTQFVRFDSDAASPREEPRAPWIEQEGPEYWDRNTQIFKTNTQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 834 - protein, 366 aa, synthetic construct
MRVMAPRTLILLLSGALALTETWAGSHSMRYFSTSVSWPGRGEPRFIAVGYVDDTQFVRFDSDAASPRGEPREPWVEQEGPEYWDRETQKYKRQAQADRVNLRKLRGYYNQSEDGSHTLQRMFGCDLGPDGRLLRGYNQFAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWKPSSQPTIPIVGIVAGLAVLAVLAVLGAMVAVVMCRRKSSGGKGGSCSQAASSNSAQGSDESLIACKA

SEQ ID NO: 835 - protein, 409 aa, synthetic construct
MERHWIFLLLLSVTAGVHSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 836 - protein, 642 aa, synthetic construct
MERHWIFLLLLSVTAGVHSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRIEGRMDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 837 - protein, 236 aa, synthetic construct
MGWSCIILFLVATATGVHSESVLTQPPSVSGAPGQRVTISCTGSSSNIGAGYDVHWYQQLPGTAPKLLIYGNSNRPSGVPDRFSVSKSGASASLAITGLQAEDEADYYCQSFDNSLSAYVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 838 - protein, 467 aa, Homo sapiens
MGWSLILLFLVAVATRVLSEVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARLPTRWVTTRYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 839 - protein, 233 aa, synthetic construct
MGWSCIILFLVATATGVHSSSELTQDPAVSVALGQTVRITCQGDSLRSYYESWYQQKPGQAPVLVIYGKNNRPSGIPDRFSGSISGNTASLTITGTQAEDEADYYCNSRDNSGDHWVFGGGTRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 840 - protein, 463 aa, Homo sapiens
MGWSLILLFLVAVATRVLSEVQLVESGGGLVQPGGSLRLSCAASGFTFSDYDMHWVRQATGKGLEWVATIDTAGDTYYPDSVRGRFTISRENAKNSLSLQMNSLRAGDTAVYFCARDVPGTGFDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 841 - protein, 127 aa, synthetic construct
QVQLVQSGAEVKKPGASVKVSCKASGYTFTRYVMHWVRQAPGQRLEWMGWINPNNDGTKYSQKFKGRVTLTRDTSASTAYMELSSLRSEDTAVYYCARGDLEILPYYDHARNAMDYWGQGTLVTVSS

SEQ ID NO: 842 - protein, 107 aa, synthetic construct
DIQMTQSPSSVSASVGDRVTITCRASENIYSYLAWYQQKPGKAPKLLVYNAKSLQSGVPSRFSGSGSGTDFTLTISSLQPEDFATYYCQHFWSTPPTFGGGTKVEIK

SEQ ID NO: 843 - protein, 127 aa, synthetic construct
QVQLVQSGAEVKKPGASVKVSCKASGYTFTRYVINWVRQATGQGLEWIGWINPNNDGTGYAQKFQGRVTLTSNKSISTAYMELSSLRSEDTAVYYCARGDLEILPYYDHARNAMDYWGQGTLVTVSS

SEQ ID NO: 844 - protein, 107 aa, synthetic construct; identical to SEQ ID NO: 842

SEQ ID NO: 845 - protein, 122 aa, synthetic construct
QVQLVQPGAEVKKPGSSVKVSCKASGYTFSSYWISWVRQAPGQGLEWMGGILPGDGSTNYAAKFQGQVTFTADTSTSTAYMQFSSLRSEDTAVYYCARSGAGGYDSGAFAYWGQGTLVTVSS

SEQ ID NO: 846 - protein, 111 aa, synthetic construct
AIQLTQSPSSLSASVGDRVTITCRASESVDTYGNSFLAWYQQKPGKPPKLLVYRTSSLESGVPSRFSGSGSGTDFTLTISSLQPEDFATYYCQQSNEDPWTFGGGTKVEIK

SEQ ID NO: 847 - protein, 122 aa, synthetic construct
QVQLVQSGAEVKKPGASVKVSCKASGYTFSSYWISWVRQAPGQGLEWIGWILPGDGSTNYAAKLQGQATFTADTSTSTAYMEFRSLRSDDTAVYYCARSGAGGYDSGAFAYWGQGTLVTVSS

SEQ ID NO: 848 - protein, 111 aa, synthetic construct
EIVLTQSPATLSLSPGERATLSCRASESVDTYGNSFLAWYQQKPGQAPRLLVYRTSNRATGIPARFSGSGSGTDFTLTISSLEPEDFAVYYCQQSNEDPWTFGGGTKVEIK

SEQ ID NO: 849 - protein, 127 aa, synthetic construct
QVQLVQSGAEVKKPGASVKVSCKASGYTFTRYVMHWVRQAPGQRLEWMGWINPANEETKYSQKFKGRVTLTRDTSASTAYMELSSLRSEDTAVYYCARADLEILPYYDHARNAMDYWGQGTLVTVSS

SEQ ID NO: 850 - protein, 107 aa, synthetic construct; identical to SEQ ID NO: 842

SEQ ID NO: 851 - protein, 127 aa, synthetic construct
QVQLVQSGAEVKKPGASVKVSCKASGYAFTRYVMHWVRQAPGQRLEWMGWINPNNEETKYSQKFKGRVTLTRDTSASTAYMELSSLRSEDTAVYYCARADLEILPYYDHARNAMDYWGQGTLVTVSS

SEQ ID NO: 852 - protein, 107 aa, synthetic construct
DIQMTQSPSSVSASAGDRVTITCRASENIYSYLAWYQQKPGKAPKLLVYSAKSLQSGVPSRFSGSGSGTDFTLTISSLQPEDFATYYCQHFWSTPPTFGGGTKVEIK

SEQ ID NO: 853 - protein, 127 aa, synthetic construct
QVQLVQSGAEVKKPGASVKVSCKASGYTFTRYVMHWVRQAPGQRLEWMGWINPNNEETKYSQKFKGGVTLTRDTSASTAYMELSSLRSEDTAVYYCARADLEILPYYDHARNAMDYWGQGTLVTVSS

SEQ ID NO: 854 - protein, 107 aa, synthetic construct
DIQMTQSPSSVSASVGDRVTITCRASENIYSHLAWYQQKPGKAPKLLVYNAKSLQSGVPSRFSGSGSGTDFTLTISSLQPEDFATYYCQHFWSTPPTFGGGTKVEIK

SEQ ID NO: 855 - protein, 127 aa, synthetic construct
QVQLVQSGAEVKKPGASVKVSCKASGYTFTRYVMHWVRQAPGQRLEWMGWINPNNEETKYSQKFKGRVTLTRDTSASTAYMELSSLRSEDTAVYYCARADLEILPYYDHARNAMDYWGQGTLVTVSS

SEQ ID NO: 856 - protein, 107 aa, synthetic construct
DIQMTQSPSSVSASVGDRVTITCRASENIYSYLAWYQQKPGKAPKLLVYQAKSLQSGVPSRFSGSGSGTDFTLTISSLQPEDFATYYCQHFWSTPPTFGGGTKVEIK

SEQ ID NO: 857 - protein, 127 aa, synthetic construct
QVQLVQSGAEVKKPGASVKVSCKASGYTFTRYVMHWVRQAPGQRLEWMGWINPNNEETKYSQKFKGRVTLTRDTSASTAYMELSSLRSEDTAVYYCARADLEILPYYDHARNAMDYWGQGTSVTVSS

SEQ ID NO: 858 - protein, 107 aa, synthetic construct; identical to SEQ ID NO: 854

SEQ ID NO: 859 - protein, 127 aa, synthetic construct
QVQLVQSGAEVKKPGASIKVSCKASGYTFTRYVMHWVRQAPGQRLEWMGWINPNNEKTKYSQKFKGRVTLTRDTSASTAYMELSSLRSEDTAVYYCARADLEILPYYDHARNAMDYWGQGTLVTVSS

SEQ ID NO: 860 - protein, 107 aa, synthetic construct; identical to SEQ ID NO: 842

SEQ ID NO: 861 - protein, 127 aa, synthetic construct
QVQLVQSGAEVKKPGASVKVSCKASGYTFTRYVMHWVRQAPGQRLEWMGWINPNNGGTEYSQKFKGRVTLTRDTSASTAYMELSSLRSEDTAVYYCARADLEILPYYDHARNAMDYWGQGTLVTVSS

SEQ ID NO: 862 - protein, 107 aa, synthetic construct
DIQMTQSPSSVSASVGDRVTITCRASENIYSYLAWYQQKPGKAPKLLVYSAKSLQSGVPSRFSGSGSGTDFTLTISSLQPEDFATYYCQHFWSTPPTFGGGTKVEIK

SEQ ID NO: 863 - protein, 127 aa, synthetic construct
QVQLVQSGAEVKKPGASVKVSCKASGYTFTRYVMHWVRQAPGQRLEWMGWINPNNGNTKYSQKFKGRVTLTRDTSASTAYMELSSLRSEDTAVYYCARADLEILPYYDHARNAMDYWGQGTLVTVSS

SEQ ID NO: 864 - protein, 107 aa, synthetic construct; identical to SEQ ID NO: 862

SEQ ID NO: 865 - protein, 127 aa, synthetic construct; identical to SEQ ID NO: 863

SEQ ID NO: 866 - protein, 107 aa, synthetic construct
DIQMTQSPSSVSASVGDRVTITCRASKNIYSLLAWYQQKPGKAPKLLVYSAKSLQSGVPSRFSGSGSGTDFTLTISSLQPEDFATYYCQHFWSTPPTFGGGTKVEIK

SEQ ID NO: 867 - protein, 127 aa, synthetic construct; identical to SEQ ID NO: 855

SEQ ID NO: 868 - protein, 107 aa, synthetic construct; identical to SEQ ID NO: 856

SEQ ID NO: 869 - protein, 127 aa, synthetic construct
QVQLVQSGAEVKKPGASVKVSCKASGYTFTRYVMHWVRQAPGQRLEWMGWINPNNGETKYSQKFKGRVTLTRDTSASTAYMELSSLRSEDTAVYYCARFDLEILPYYDHARNAMDYWGQGTLVTVSS

SEQ ID NO: 870 - protein, 107 aa, synthetic construct
YIQMTQSPSSVSASVGDRVTITCRASENIYSYLAWYQQKPGKAPKLLVYSAKSLQSGVPSRFSGSGSGTDFTLTISSLQPEDFATYYCQHFWSTPPTFGGGTKVEIK

SEQ ID NO: 871 - protein, 127 aa, synthetic construct
QVQLVQSGAEVKKPGASVKVSCKASGYTFTRYVMHWVRQAPGQRLEWMGWINPHNEGTKYSQKFKGRVTLTRDTSASTAYMELSSLRSEDTAVYYCARGDLEILPYYDHARNAMDYWGQGTLVTVSS

SEQ ID NO: 872 - protein, 107 aa, synthetic construct
DIQMTQSPSSVSASVGDRVTITCRASENIYNYLAWYQQKPGKAPKLLVYSAKSLQSGVPSRFSGSVSGTDFTLTISSLQPEDFATYYCQHFWSTPPTFGGGTKVEIK

SEQ ID NO: 873 - protein, 127 aa, synthetic construct
QVQLVQSGAEVKKPGASVKVSCKASGYTFTRYVMHWVRQAPGQRLEWMGWINPHNENTKYSQKFKGRVTLTRDTSASTAYMELSSLRSEDTAVYYCARGDLEILPYYDHARNAMDYWGQGTLVTVSS

SEQ ID NO: 874 - protein, 107 aa, synthetic construct; identical to SEQ ID NO: 862

SEQ ID NO: 875 - protein, 127 aa, synthetic construct
QVQLVQSGAEVKKPGASVKVSCKASGYTFTRYVMHWVRQAPGQRLEWMGWINPNNEKTKYSQKFKGRVTLTRDTSASTAYMELSSLRSEDAAVYYCARGDLEILPYYDHARNAMDYWGQGTLVTVSS

SEQ ID NO: 876 - protein, 107 aa, synthetic construct; identical to SEQ ID NO: 862

SEQ ID NO: 877 - protein, 127 aa, synthetic construct
QVQLVQSGAEVKKPGASVKVSCKASGYTFTRYVMHWVRQAPGQRLEWMGWINPNNERTKYSQKFKGRVTLTRDTSASTAYMELSSLRSEDTAVYYCARGDLEILPYYDHARNAMDYWGQGTLVTVSS

SEQ ID NO: 878 - protein, 107 aa, synthetic construct
DIQMTQSPSSVSASVGDRVTITCRASENIYNYLAWYQQKPGKAPKLLVYSAKSLQSGVPSRFSGSGSGTDFTLTISSLQPEDFATYYCQHFWSTPPTFGGGTKVEIK

SEQ ID NO: 879 - protein, 127 aa, synthetic construct
QVQLVQSGAEVKKPGASVKVSCKASGYTFTRYVMHWVRQAPGQRLEWMGWINPNNEVTKYSQKFKGRVTLTRDTSASTAYMELSSLRSEDTAVYYCARGDLEILPYYDHARNAMDYWGQGTLVTVSS

SEQ ID NO: 880 - protein, 107 aa, synthetic construct; identical to SEQ ID NO: 862

SEQ ID NO: 881 - protein, 127 aa, synthetic construct
QVQLVQSGAEVKKPGASVKVSCKASGYTFTRYVMHWVRQAPGQRLEWMGWINPNNGNTKYSQKFKGRVTLTRDTSASTAYMELSSLRSEDTAVYYCARGDLEILPYYDHARNAMDYWGQGTLVTVSS

SEQ ID NO: 882 - protein, 107 aa, synthetic construct; identical to SEQ ID NO: 862

SEQ ID NO: 883 - protein, 127 aa, synthetic construct
QVQLVQSGAEVKKPGASVKVSCKASGYTFTRYVMHWVRQAPGQRLEWMGWINPQNDNTKYSQKFKGRVTLTRDTSASTAYMELSSLRSEDTAVYYCARGDLEILPYYDHARNAMDYWGQGTLVTVSS

SEQ ID NO: 884 - protein, 107 aa, synthetic construct; identical to SEQ ID NO: 856

SEQ ID NO: 885 - protein, 127 aa, synthetic construct
QVQLVQSGAEVKKPGASVKVSCKASGYTFTRYVMHWVRQAPGQRLEWMGWINPQNEATKYSQKFKGRVTLTRDTSASTAYMELSSLRSEDTAVYYCARGDLEILPYYDHARNAMDYWGQGTLVTVSS

SEQ ID NO: 886 - protein, 107 aa, synthetic construct; identical to SEQ ID NO: 862

SEQ ID NO: 887 - protein, 127 aa, synthetic construct; identical to SEQ ID NO: 885

SEQ ID NO: 888 - protein, 107 aa, synthetic construct
DIQMTQSPSSVSASVGDRVTITCRASENIYSYLAWYQQKPGKAPKLLVYSDKSLQSGVPSRFSGSGSGTDFTLTISSLQPEDFATYYCQHFWSTPPTFGGGTKVEIK

SEQ ID NO: 889 - protein, 127 aa, synthetic construct
QVQLVQSGAEVKKPGASVKVSCKASGYTFTRYVMHWVRQAPGQRLEWMGWINPQNEETKYSQKFKGRVTLTRDTSASTAYMELSSLRSEDTAVYYCARGDLEILPYYDHARNAMDYWGQGTLVTVSS

SEQ ID NO: 890 - protein, 107 aa, synthetic construct; identical to SEQ ID NO: 842

SEQ ID NO: 891 - protein, 127 aa, synthetic construct
QVQLVQSGAEVKKPGASVKVSCKASGYTFTRYVMHWVRQAPGQRLEWMGWINPQNEGTKYSQKFKGRVTLTRDTSASTAYMELSSLRSEDTAVYYCARGDLEILPYYDHARNAMDYWGQGTLVTVSS

SEQ ID NO: 892 - protein, 107 aa, synthetic construct; identical to SEQ ID NO: 878

SEQ ID NO: 893 - protein, 127 aa, synthetic construct; identical to SEQ ID NO: 891

SEQ ID NO: 894 - protein, 107 aa, synthetic construct; identical to SEQ ID NO: 862

SEQ ID NO: 895 - protein, 127 aa, synthetic construct
QVQLVQSGAEVKKPGASVKVSCKASGYTFTRYVMHWVRQAPGQRLEWMGWINPQNEKTKYSQKFKGRVTLTRDTSASTAYMELSSLRSEDTAVYYCARGDLEILPYYDHARNAMDYWGQGTLVTVSS

SEQ ID NO: 896 - protein, 107 aa, synthetic construct
DIQMTQSPSSVSASVGDRVTITCRASENIYNYLAWYQQKPGKAPKLLVYQAKSLQSGVPSRFSGSGSGTDFTLTISSLQPEDFATYYCQHFWSTPPTFGGGTKVEIK

SEQ ID NO: 897 - protein, 127 aa, synthetic construct; identical to SEQ ID NO: 895

SEQ ID NO: 898 - protein, 107 aa, synthetic construct; identical to SEQ ID NO: 856

SEQ ID NO: 899 - protein, 127 aa, synthetic construct; identical to SEQ ID NO: 895

SEQ ID NO: 900 - protein, 107 aa, synthetic construct; identical to SEQ ID NO: 862

SEQ ID NO: 901 - protein, 127 aa, synthetic construct
QVQLVQSGAEVKKPGASVKVSCKASGYTFTRYVNAWVRQAPGQRLEWMGWITPQNEATKYSQKFKGRVTLTRDTSASTAYMELSSLRSEDTAVYYCARGDLEILPYYDHARNAMDYWGQGTLVTVSS

SEQ ID NO: 902 - protein, 107 aa, synthetic construct; identical to SEQ ID NO: 862

SEQ ID NO: 903 - protein, 127 aa, synthetic construct
QVQLVQSGAEVKKPGASVKVSCKASGYTFTRYVMHWVRQAPGQRPEWMGWINPEREETKYSQKFKGRVTLTRDTSASTAYMELSSLRSEDTAVYYCARGDLEILPYYDHVRNAMDYWGQGTLVTVSS

SEQ ID NO: 904 - protein, 107 aa, synthetic construct; identical to SEQ ID NO: 856

SEQ ID NO: 905 - protein, 127 aa, synthetic construct
QVQLVQSGAEVKKPGASVKVSCKASGYTFTRYVMHWVRQAPGQRLEWMGWINPANEETKYSQKFKGRVTLTRDTSASTAYMELSSLRSEDTAVYYCARSDLEILPYYDHARNAMDYWGQGTLVTVSS

SEQ ID NO: 906 - protein, 107 aa, synthetic construct; identical to SEQ ID NO: 842

SEQ ID NO: 907 - protein, 127 aa, synthetic construct
QVQLVQSGAEVKKPGASVKVSCKASGYTFTRYVMHWVRQAPGQRLEWMGWINPANGATKYSQKFKGRVTLTRDTSASTAYMELSSLRSEDTAVYYCARSDLEILPYYDHARNAMDYWGQGTLVTVSS

SEQ ID NO: 908 - protein, 107 aa, synthetic construct; identical to SEQ ID NO: 842

SEQ ID NO: 909 - protein, 127 aa, synthetic construct; identical to SEQ ID NO: 907

SEQ ID NO: 910 - protein, 107 aa, synthetic construct
YIQMTQSPSSVSASVGDRVTITCRASENIYSYLAWYQQKPGKAPKLLVYNAKSLQSGVPSRFSGSGSGTDFTLTISSLQPEDFATYYCQHFWSTPPTFGGGTKVEIK

SEQ ID NO: 911 - protein, 127 aa, synthetic construct
QVQMVQSGAEVKKPGASVKVSCKASGYTFTRYVMHWVRQAPGQRLEWMGWINPANGATKYSQKFKGRVTLTRDTSASTAYMELSSLRSEDTAVYYCARSDLEILPYYDHARNAMDYWGQGTLVTVSS

SEQ ID NO: 912 - protein, 107 aa, synthetic construct; identical to SEQ ID NO: 842

SEQ ID NO: 913 - protein, 127 aa, synthetic construct
QVQLVQSGAEVKKPGASVKVSCKASGYTFTRYVMHWVRQAPGQRLEWMGWINPANGKTKYSQKFKGRVTLTRDTSASTAYMELSSLRSEDTAVYYCARSDLEILPYYDHARNAMDYWGQGTLVTVSS

SEQ ID NO: 914 - protein, 107 aa, synthetic construct; identical to SEQ ID NO: 856

SEQ ID NO: 915 - protein, 127 aa, synthetic construct
QVQLVQSGAEVKKPGASVKVSCKASGYTFTRYVMHWVRQAPGQRLEWMGWINPENEETKYSQKFKGRVTLTRDTSASTAYMELSSLRSEDTAVYYCARSDLEILPYYDHARNAMDYWGQGTLVTVSS

SEQ ID NO: 916 - protein, 107 aa, synthetic construct; identical to SEQ ID NO: 842

SEQ ID NO: 917 - protein, 127 aa, synthetic construct
QVQLVQSGAEVKKPGASVKVSCKASGYTFTRYVMHWVRQAPGQRLEWMGWINPHNEETKYSQKFKGRVTLTRDTSASTAYMELSSLRSEDTAVYYCARSDLEILPYYDHARNAMDYWGQGTLVTVSS

SEQ ID NO: 918 - protein, 107 aa, synthetic construct; identical to SEQ ID NO: 896

SEQ ID NO: 919 - protein, 127 aa, synthetic construct; identical to SEQ ID NO: 917

SEQ ID NO: 920 - protein, 107 aa, synthetic construct; identical to SEQ ID NO: 856

SEQ ID NO: 921 - protein, 127 aa, synthetic construct
QVQLVQSGAEVKKPGASVKVSCKASGYTFTRYVMHWVRQAPGQRLEWMGWINPHNEETRYSQKFKGRVTLTRDTSASTAYMELSSLRSEDTAVYYCARSDLEILPYYDHARNAMDYWGQGTLVTVSS

SEQ ID NO: 922 - protein, 107 aa, synthetic construct; identical to SEQ ID NO: 842

SEQ ID NO: 923 - protein, 126 aa, synthetic construct
QVQLVQSGAEVQKPGASVVSCKASGYTFTRYVMHWVRQAPGQRLEWMGWINPHNEETKYSQKFKGRVTLTRDTSASTAYMELSSLRSEDTAVYYCARSDLEILPYYDHARNAMDYWGQGTLVAVSS

SEQ ID NO: 924 - protein, 107 aa, synthetic construct
DIQMTPSPSSVSASVGDRVTITCRASENIYSYLAWYQQKPGKAPKLLVYSAKSLQSGVPSRFSGSGSGTDFTLTISSLQPEDFATYYCQHFWSTPPTFGGGTKVEIK

SEQ ID NO: 925 - protein, 127 aa, synthetic construct
QVQLVQSGAEVKKPGASVKVSCKASGYTFTRYVMHWVRQAPGQRLEWMGWINPHNEGTKYSQKFKGRVTLTRDTSASTAYMELSSLRSEDTAVYYCARSDLEILPYYDHARNAMDYWGQGTLVTVSS

SEQ ID NO: 926 - protein, 107 aa, synthetic construct
DIQMTQSPSSVSASVGDRVTITCRASENIYSYLAWYQQKPGKAPKLLVYNAKSLQSGVPSRFSGSGSGTDFTLTISSLQPEDFATYYCQHFWSAPPTFGGGTKVEIK

SEQ ID NO: 927 - protein, 127 aa, synthetic construct
QVQLVQSGAEVKKPGASVKVSCKASGYTFTRYVMHWVRQAPGQRLEWMGWINPHNGGTKYSQKFKGRVTLTRDTSASTAYMELSSLRSEDTAVYYCARSDLEILPYYDHARNAMDYWGQGTLVTVSS

SEQ ID NO: 928 - protein, 107 aa, synthetic construct; identical to SEQ ID NO: 862

SEQ ID NO: 929 - protein, 127 aa, synthetic construct; identical to SEQ ID NO: 927

SEQ ID NO: 930 - protein, 107 aa, synthetic construct; identical to SEQ ID NO: 870

SEQ ID NO: 931 - protein, 127 aa, synthetic construct
QVQLVQSGAEVKKPGASVKVSCKASGYTFTRYVMHWVRQAPGQRPEWMGWINPKNEGTKYSQKFKGRVTLTRDTSASTAYMELSSLRSEDTAVYYCARSDLEILPYYDHARNAMDYWGQGTLVTVSS

SEQ ID NO: 932 - protein, 107 aa, synthetic construct; identical to SEQ ID NO: 870

SEQ ID NO: 933 - protein, 127 aa, synthetic construct
QVQLVQSGAEVKKPGASVKVSCKASGYTFTRYVMHWVRQAPGQRLEWMGWINPNNEETKYSQKFKGRVTLTRDTSASTAYMELSSLRSEDTAVYYCARSDLEILPYYDHARNAMDYWGQGTLVTVSS

SEQ ID NO: 934 - protein, 107 aa, synthetic construct
DIQMTQSPSSVSASVGDRVTITCRASENIHSYLAWYQQKPGKAPKLLVYNAKSLQSGVPSRFSGSGSGTDFTLTISSLQPEDFATYYCQHFWSTPPTFGGGTKVEIK

SEQ ID NO: 935 - protein, 127 aa, synthetic construct; identical to SEQ ID NO: 933

SEQ ID NO: 936 - protein, 107 aa, synthetic construct; identical to SEQ ID NO: 842

SEQ ID NO: 937 - protein, 127 aa, synthetic construct; identical to SEQ ID NO: 933

SEQ ID NO: 938 - protein, 107 aa, synthetic construct; identical to SEQ ID NO: 862

SEQ ID NO: 939 - protein, 127 aa, synthetic construct
QVQLVQSGAEVKKPGASVKVSCKASGYTFTRYVMHWVRQAPGQRLEWMGWINPNNEKTKYSQKFKGRVTLTRDTSASTAYMELSSLRSEDTAVYYCARSDLEILPYYDHARNAMDYWGQGTLVTVSS

SEQ ID NO: 940 - protein, 107 aa, synthetic construct; identical to SEQ ID NO: 862

SEQ ID NO: 941 - protein, 127 aa, synthetic construct
QVQLVQSGAEVKKPGASVKVSCKASGYTFTRYVMHWVRQAPGQRLEWMGWINPNNGEAKYSQKFKGRVTLTRDTSASTAYMELSSLRSEDTAVYYCARSDLEILPYYDHARNAMDYWGQGTLVTVSS

SEQ ID NO: 942 - protein, 107 aa, synthetic construct
YIQMTQSPSSVSASVGDRVTITCRASENIYSYLAWYQQKPGKAPKLLVYSVKSLQSGVPSRFSGSGSGTDFTLTISSLQPEDFATYYCQHFWSTPPTFGGGTKVEIK

SEQ ID NO: 943 - protein, 127 aa, synthetic construct
QVQLVQSGAEVKKPGASVKVSCKASGYTFTRYVMHWVRQAPGQRLEWMGWINPNNGETKYSQKFKGGVTLTRDTFGSTAYMELSSLRSEDTAVYYCARSDLEILPYYDHARNAMDYWGQGTLVTVSS

SEQ ID NO: 944 - protein, 107 aa, synthetic construct; identical to SEQ ID NO: 870

SEQ ID NO: 945 - protein, 127 aa, synthetic construct
QVQLVQSGAEVKKPGASVKVSCKASGYTFTRYVMHWVRQAPGQRLEWMGWINPNNGETKYSQKFKGRVTLTRDTSASTAYMELSSLRSEDTAVYYCARSDLEILPYYDHARNAMDYWGQGTLVTVSS

SEQ ID NO: 946 - protein, 107 aa, synthetic construct; identical to SEQ ID NO: 862

SEQ ID NO: 947 - protein, 127 aa, synthetic construct; identical to SEQ ID NO: 945

SEQ ID NO: 948 - protein, 107 aa, synthetic construct
DIQMTQSPSSVSASVGDRVTITCRASKNIYSYLAWYQQKPGKAPKLLVYSAKSLQSGVPSRFSGSGSGTDFTLTISSLQPEDFATYYCQHFWSTPPTFGGGTKVEIK

SEQ ID NO: 949 - protein, 127 aa, synthetic construct
QVQLVQSGAEVKKPGPSVKVSCKASGYTFTRYVMHGVRQAPGQRLEGMGWINPNNGETKYSQKFKGRVTLTRDTSASTAYMELSSLRSEDTAVYYCARSDLEILPYYDHARNAMDYWGQGTLVTVSS

SEQ ID NO: 950 - protein, 107 aa, synthetic construct
YIQMTQSPSSVSASVGDRVTITCRASENIYSYLAWYQQKPGKAPKLLVYSVKSLQSGVPSRFSGSGSGRDFTLTISSLQPEDFATYYCQHFGSTPPTFGGGTKVEIK

SEQ ID NO: 951 - protein, 127 aa, synthetic construct
QVQLVQSGAEVQKPGASVKVSCKASGYTFTRYVMHWVRQAPGQRLEWMGWINPNNGETKYSQKFKGRVTLTRDTSASTAYMELSSLRSEDTAVYYCARSDLEILPYYDHARNAMDYWGQGTLVTVSS

SEQ ID NO: 952 - protein, 107 aa, synthetic construct; identical to SEQ ID NO: 862

SEQ ID NO: 953 - protein, 127 aa, synthetic construct
QVQLVQSGAEVKKPGASVKVSCKASGYTFTRYVMHWVRQAPGQRLEWMGWINPQNEATKYSQKFKGRVTLTRDTSASTAYMELSSLRSEDTAVYYCARSDLEILPYYDHARNAMDYWGQGTLVTVSS

SEQ ID NO: 954 - protein, 107 aa, synthetic construct; identical to SEQ ID NO: 842

SEQ ID NO: 955 - protein, 127 aa, synthetic construct
QVQLVQSGAEVKKPGASVKVSCKASGYTFTRYVMHWVRQAPGQRLEWMGWINPQNERTKYSQKFKGRVTLTRDTSASTAYMELSSLRSEDTAVYYCARSDLEILPYYDHARNAMDYWGQGTLVTVSS

SEQ ID NO: 956 - protein, 107 aa, synthetic construct; identical to SEQ ID NO: 862

SEQ ID NO: 957 - protein, 127 aa, synthetic construct
QVQLVQSGAEVKKPGASVKVSCKASGYTFTRYVMHWVRQAPGQRLEWMGWINPNNGETKYSQKFKGRVTLPRDTSASTAYMELSSLRSEDTAVYYCARSDLEILPYYDHARNPMDYWGQGTLVTVSS

SEQ ID NO: 958 - protein, 107 aa, synthetic construct; identical to SEQ ID NO: 870

SEQ ID NO: 959 - protein, 127 aa, synthetic construct
QVQLVQSGAEVKKPGASVKVSCKASGYTFTRYVMHWVRQAPGQRLEWMGWINPNNGETKYSQKFKGRVTLPRDTSASTAYMELSSLRSEDTAVYYSARSDLEILPYYDHARNPMDYWGQGTLVTVSS

SEQ ID NO: 960 - protein, 107 aa, synthetic construct; identical to SEQ ID NO: 870

SEQ ID NO: 961 - protein, 127 aa, synthetic construct
QVQLVQSGAEVQKPGASVKVSCKASGYTFTRYVMHWVRQAPGQRLEWMGWINPNNGETKYSQKFKGRVTLPRDTSASTAYMELSSLRSEDTAVYYCARSDLEILPYYDHARNPMDYWGQGTLVTVSS

SEQ ID NO: 962 - protein, 107 aa, synthetic construct; identical to SEQ ID NO: 870

SEQ ID NO: 963 - protein, 127 aa, synthetic construct
QVQLVQSGAEVKKPGASVKVSCKASGYTFTRYVMHWVRQAPGQRLEWMGWINPHNGNTKYSQKFKGRVTLTRDTSASTAYMELSSLRSEDTAVYYCARVDLEILPYYDHARNAMDYWGQGTLVTVSS

SEQ ID NO: 964 - protein, 107 aa, synthetic construct
DIQMTQSPSSVSASVGDRVTITCRASENIYSYLAWYQQKPGKAPKLLVYQAKSLQSGVPSRFSGSGSGTDFTLTISSLQPEDFATYYCQHFWSTPPTFGGGTKVETK

SEQ ID NO: 965 - protein, 127 aa, synthetic construct
QVQLVQSGAEVKKPGASVKVSCKASGYTFTRYVMHWVRQAPGQRLEWMGWINPQNEETKYSQKFKGRVTLTRDTSASTAYMELSSLRSEDTAVYYCARVDLEILPYYDHARNAMDYWGQGTLVTVSS

SEQ ID NO: 966 - protein, 107 aa, synthetic construct; identical to SEQ ID NO: 862

SEQ ID NO: 967 - protein, 127 aa, synthetic construct
QVQLVQSGAEVKKPGASVKVSCKASGYTFTRYVMHWVRQAPGQRPEWMGWINPQNEGTKYSQKFKGRVTLTRDTSASTAYMELSSLRSEDTAVYYCARVDLEILPYYDHARNAMDYWGQGTLVTVSS

SEQ ID NO: 968 - protein, 107 aa, synthetic construct; identical to SEQ ID NO: 856

SEQ ID NO: 969 - protein, 122 aa, synthetic construct
QVQLVQPGAEVKKPGSSVKVSCKASGNTFSNYWISWVRQAPGQGLEWMGGILPGDESTNYAAKFQGQVTFTADTSTSTAYMQFSSLRSEDTAVYYCARSGAGGYESGSFAYWGQGTLVTVSS

SEQ ID NO: 970 - protein, 111 aa, synthetic construct
AIQLTQSPSSLSASVGDRVTITCRASKSIDSNGDSFLAWYQQKPGKPPKLLVYRTSSLESGVPSRFSGSGSGTDFTLTISSLQPEDFATYYCQQSNEDPWTFGGGTKVEIK

SEQ ID NO: 971 - protein, 122 aa, synthetic construct
QVQLVQPGAEVKKPGSSVKVSCKASGYTFSSYWISWVRQAPGQGLEWMGGILPGSGSTNYAAKFQGQVTFTADTSTSTAYMQFSSLRSEDTAVYYCARSGPGKRDSGGFAYWGQGTLVTVSS

SEQ ID NO: 972 - protein, 111 aa, synthetic construct
AIQLTQSPSSLSASVGDRVTITCRASESIDSKGYSFLAWYQQKPGKPPKLLVYRTSSLESGVPSRFSGSGSGTDFTLTISSLQPEDFATYYCQQSNEDPWTFGGGTKVEIK

SEQ ID NO: 973 - protein, 122 aa, synthetic construct
QVQLVQPGAEVKKPGSSVKVSCKASGNTFSNYWISWVRQAPGQGLEWMGGILPGEGSTNYAAKFQGQVTFTADTSTSTAYMQFSSLRSEDTAVYYCARSGRGGYESGAFAYWGQGTLVTVSS

SEQ ID NO: 974 - protein, 111 aa, synthetic construct
AIQLTQSPSSLSASVGDRVTITCRASESVDTEGYRFLAWYQQKPGKPPKLLVYRTSSLESGVPSRFSGSGSGTDFTLTISSLQPEDFATYYCQQSNEDPWTFGGGTKVEIK

SEQ ID NO: 975 - protein, 122 aa, synthetic construct
QVQLVQPGAEVKKPGSSVKVSCKASGYTFSNSWISWVRQAPGQGLEWMGGILPGEGSTNYAAKFQGQVTFTADTSTSTAYMQFSSLRSEDTAVYYCARSGRGHYDSGRFAYWGQGTLVTVSS

SEQ ID NO: 976 - protein, 111 aa, synthetic construct
AIQLTQSPSSLSASVGDRVTITCRASESVDSHGYRFLAWYQQKPGKPPKLLVYRTSSLESGVPSRFSGSGSGTDFTLTISSLQPEDFATYYCQQSNEDPWTFGGGTKVEIK

SEQ ID NO: 977 - protein, 122 aa, synthetic construct
QVQLVQPGAEVKKPGSSVKVSCKASGYTFSSYWISWVRQAPGQGLEWMGGILPGSGSTNYAAKFQGQVTFTADTSTSTAYMQFSSLRSEDTAVYYCARSGAGKRDSGAFAYWGQGTLVTVSS

SEQ ID NO: 978 - protein, 111 aa, synthetic construct
AIQLTQSPSSLSASVGDRVTITCRASESIDTKGYSFLAWYQQKPGKPPKLLVYRTSSLESGVPSRFSGSGSGTDFTLTISSLQPEDFATYYCQQSNEDPWTFGGGTKVEIK

SEQ ID NO: 979 - protein, 127 aa, synthetic construct; identical to SEQ ID NO: 855

SEQ ID NO: 980 - protein, 107 aa, synthetic construct; identical to SEQ ID NO: 854

SEQ ID NO: 981 - protein, 107 aa, synthetic construct
DIQMTQSPSSVSASVGDRVTITCRASGNIHNYLAWYQQKPGKAPKLLVYNAKSLQSGVPSRFSGSGSGTDFTLTISSLQPEDFATYYCQHFWSSPPTFGGGTKVEIK

SEQ ID NO: 982 - protein, 107 aa, synthetic construct
DIQMTQSPSSVSASVGDRVTITCRASGNIHNYLAWYQQKPGKAPKLLVYNAKSLQSGVPSRFSGSGSGTDFTLTISSLQPEDFATYYCQHFWSTPPTFGGGTKVEIK

SEQ ID NO: 983 - protein, 107 aa, synthetic construct
DIQMTQSPSSVSASVGDRVTITCRASGNIHNYLAWYQQKPGKAPKLLVYNAKTLAEGVPSRFSGSGSGTDFTLTISSLQPEDFATYYCQHFWSSPPTFGGGTKVEIK

SEQ ID NO: 984 - protein, 107 aa, synthetic construct
DIQMTQSPSSVSASVGDRVTITCRASGNIHNYLAWYQQKPGKAPKLLVYNAKTLASGVPSRFSGSGSGTDFTLTISSLQPEDFATYYCQHFWSSPPTFGGGTKVEIK

SEQ ID NO: 985 - protein, 127 aa, synthetic construct
QVQLVQSGAEVKKPGASVKVSCKASGYTFTRYVMHWVRQAPGQRPEWMGWINPNNEETKYSQKFKGRVTLTRDTSASTAYMELSSLRSEDTAVYYCARADLEILPYYDHARNAMDYWGQGTLVTVSS

SEQ ID NO: 986 - protein, 107 aa, synthetic construct
DIQMTQSPSSVSASVGDRVTITCRASKNIYSHLAWYQQKPGKAPKLLVYNAKSLQSGVPSRFSGSGSGTDFTLTISSLQPEDFATYYCQHFWSTPPTFGGGTKVEIK

SEQ ID NO: 987 - protein, 127 aa, synthetic construct
QVQLVQSGAEVKKPGASVKVSCKASGYTFTRYVMHWVRQAPGQRLEWMGWINPNNEETKYSQKFKGRVTLTRDTSASTAYMELSSLRSEDTAVYYCARADLEILPYYDHARYAMDYWGQGTLVTVSS

SEQ ID NO: 988 - protein, 107 aa, synthetic construct; identical to SEQ ID NO: 854

SEQ ID NO: 989 - protein, 107 aa, synthetic construct; identical to SEQ ID NO: 981

SEQ ID NO: 990 - protein, 107 aa, synthetic construct; identical to SEQ ID NO: 982

SEQ ID NO: 991 - protein, 107 aa, synthetic construct; identical to SEQ ID NO: 983

SEQ ID NO: 992 - protein, 107 aa, synthetic construct; identical to SEQ ID NO: 984

SEQ ID NO: 993 - protein, 127 aa, synthetic construct
QVQLVQSGAEVKKPGASVKVSCKASGYTFTRYVMHWVRQAPGQRLEWMGWINPQNEETKYSQKFKGRVTLTRDTSASTAYMELSSLRSEDTAVYYCARCDLEILPYYDHARNAMDYWGQGTLVTVSS

SEQ ID NO: 994 - protein, 107 aa, synthetic construct; identical to SEQ ID NO: 862

SEQ ID NO: 995 - protein, 127 aa, synthetic construct; identical to SEQ ID NO: 905

SEQ ID NO: 996 - protein, 107 aa, synthetic construct; identical to SEQ ID NO: 862

SEQ ID NO: 997 - protein, 127 aa, synthetic construct; identical to SEQ ID NO: 917

SEQ ID NO: 998 - protein, 107 aa, synthetic construct; identical to SEQ ID NO: 862

SEQ ID NO: 999 - protein, 127 aa, synthetic construct; identical to SEQ ID NO: 933

SEQ ID NO: 1000 - protein, 107 aa, synthetic construct; identical to SEQ ID NO: 854

SEQ ID NO: 1001 - protein, 107 aa, synthetic construct; identical to SEQ ID NO: 981

SEQ ID NO: 1002 - protein, 107 aa, synthetic construct; identical to SEQ ID NO: 982

SEQ ID NO: 1003 - protein, 107 aa, synthetic construct; identical to SEQ ID NO: 983

SEQ ID NO: 1004 - protein, 107 aa, synthetic construct; identical to SEQ ID NO: 984

SEQ ID NO: 1005 - protein, 127 aa, synthetic construct; identical to SEQ ID NO: 933

SEQ ID NO: 1006 - protein, 107 aa, synthetic construct; identical to SEQ ID NO: 870

SEQ ID NO: 1007 - protein, 127 aa, synthetic construct
QVQLVQSGAEVKKPGASVKVSCKASGYTFTRYVMHWVRQAPGQRLEWMGWINPQNEETKYSQKFKGRVTLTRDASASTAYMELSSLRSEDTAVYYCARSDLEILPYYDHARNAMDYWGQGTLVTVSS

SEQ ID NO: 1008 - protein, 107 aa, synthetic construct; identical to SEQ ID NO: 862

SEQ ID NO: 1009 - protein, 107 aa, synthetic construct; identical to SEQ ID NO: 981

SEQ ID NO: 1010 - protein, 107 aa, synthetic construct; identical to SEQ ID NO: 982

SEQ ID NO: 1011 - protein, 127 aa, synthetic construct; identical to SEQ ID NO: 945

SEQ ID NO: 1012 - protein, 107 aa, synthetic construct; identical to SEQ ID NO: 870

SEQ ID NO: 1013 - protein, 127 aa, synthetic construct
QVQLVQSGAEVKKPGASVKVSCKASGYTFTRYVMHWVRQAPGQRLEWIGYINPNNEETKYNEKFKGKVTLTSDKSASTAYMELSSLRSEDTAVYYCARADLEILPYYDHARNAMDYWGQGTLVTVSS

SEQ ID NO: 1014 - protein, 107 aa, synthetic construct
DIQMTQSPSSVSASVGDRVTITCRASENIYSHLAWYQQKPGKAPKLLVYNAKSLAEGVPSRFSGSGSGTDFTLTISSLQPEDFATYYCQHFWSTPPTFGGGTKVEIK

SEQ ID NO: 1015 - protein, 127 aa, synthetic construct
QVQLVQSGAEVKKPGASVKVSCKASGYTFTRYVMHWVRQAPGQRLEWIGYINPNNEETKYNEKFKGKVTLTSDKSASTAYMELSSLRSEDTAVYYCARADLEILPYYDHARYAMDYWGQGTLVTVSS

SEQ ID NO: 1016 - protein, 107 aa, synthetic construct; identical to SEQ ID NO: 1014

SEQ ID NO: 1017 - protein, 127 aa, synthetic construct
QVQLVQSGAEVKKPGASVKVSCKASGYTFTRYVMHWVRQAPGQRLEWIGYINPQNEETKYNEKFKGKVTLTSDKSASTAYMELSSLRSEDTAVYYCARCDLEILPYYDHARNAMDYWGQGTLVTVSS

SEQ ID NO: 1018 - protein, 107 aa, synthetic construct
DIQMTQSPSSVSASVGDRVTITCRASENIYSYLAWYQQKPGKAPKLLVYSAKSLAEGVPSRFSGSGSGTDFTLTISSLQPEDFATYYCQHFWSTPPTFGGGTKVEIK

SEQ ID NO: 1019 - protein, 122 aa, synthetic construct
QVQLVQSGAEVKKPGASVKVSCKASGYTFTSYWIEWVRQAPGQGLEWIGEILPGDGSTNYNAKFKGRVTFTRDTSTSTAYMELSSLRSEDTAVYYCARSGAGGYDSGAFAYWGQGTLVTVSS

SEQ ID NO: 1020 - protein, 111 aa, synthetic construct
EIVLTQSPATLSLSPGERATLSCRASESVDTYGNSFMHWYQQKPGQAPRLLVYRTSNLESGIPARFSGSGSGTDFTLTISSLEPEDFAVYYCQQSNEDPWTFGGGTKVEIK

SEQ ID NO: 1021 - protein, 122 aa, synthetic construct
QVQLVQSGAEVKKPGASVKVSCKASGYTFTSYWIEWVRQAPGQGLEWIGEILPGDGSTNYNAKFKGRVTFTTDTSTSTAYMELRSLRSDDTAVYYCARSGAGGYDSGAFAYWGQGTLVTVSS

SEQ ID NO: 1022 - protein, 111 aa, synthetic construct
AIQLTQSPSSLSASVGDRVTITCRASESVDTYGNSFMHWYQQKPGKAPKLLVYRTSNLESGVPSRFSGSGSGTDFTLTISSLQPEDFATYYCQQSNEDPWTFGGGTKVEIK

SEQ ID NO: 1023 - protein, 122 aa, synthetic construct
QVQLVQSGAEVKKPGASVKVSCKASGYTFTSYWIEWVRQAPGQGLEWIGEILPGSGSTNYNAKFKGRVTFTRDTSTSTAYMELSSLRSEDTAVYYCARSGPGKRDSGGFAYWGQGTLVTVSS

SEQ ID NO: 1024 - protein, 111 aa, synthetic construct
EIVLTQSPATLSLSPGERATLSCRASESIDSKGYSFMHWYQQKPGQAPRLLVYRTSNLESGIPARFSGSGSGTDFTLTISSLEPEDFAVYYCQQSNEDPWTFGGGTKVEIK

SEQ ID NO: 1025 - protein, 122 aa, synthetic construct
QVQLVQSGAEVKKPGASVKVSCKASGYTFTSYWIEWVRQAPGQGLEWIGEILPGSGSTNYNAKFKGRVTFTTDTSTSTAYMELRSLRSDDTAVYYCARSGPGKRDSGGFAYWGQGTLVTVSS

SEQ ID NO: 1026 - protein, 111 aa, synthetic construct
AIQLTQSPSSLSASVGDRVTITCRASESIDSKGYSFMHWYQQKPGKAPKLLVYRTSNLESGVPSRFSGSGSGTDFTLTISSLQPEDFATYYCQQSNEDPWTFGGGTKVEIK

SEQ ID NO: 1027 - protein, 122 aa, synthetic construct
QVQLVQSGAEVKKPGASVKVSCKASGYTFTNYWIEWVRQAPGQGLEWIGEILPGEGSTNYNAKFKGRVTFTRDTSTSTAYMELSSLRSEDTAVYYCARSGRGGYESGAFAYWGQGTLVTVSS

SEQ ID NO: 1028 - protein, 111 aa, synthetic construct
EIVLTQSPATLSLSPGERATLSCRASESVDTEGYRFMHWYQQKPGQAPRLLVYRTSNLESGIPARFSGSGSGTDFTLTISSLEPEDFAVYYCQQSNEDPWTFGGGTKVEIK

SEQ ID NO: 1029 - protein, 122 aa, synthetic construct
QVQLVQSGAEVKKPGASVKVSCKASGYTFSNSWIEWVRQAPGQGLEWIGEILPGEGSTNYNAKFKGRVTFTRDTSTSTAYMELSSLRSEDTAVYYCARSGRGHYDSGRFAYWGQGTLVTVSS

SEQ ID NO: 1030 - protein, 111 aa, synthetic construct
EIVLTQSPATLSLSPGERATLSCRASESVDSHGYRFMHWYQQKPGQAPRLLVYRTSNLESGIPARFSGSGSGTDFTLTISSLEPEDFAVYYCQQSNEDPWTFGGGTKVEIK

SEQ ID NO: 1031 - protein, 122 aa, synthetic construct
QVQLVQSGAEVKKPGASVKVSCKASGYTFTSYWIEWVRQAPGQGLEWIGEILPGSGSTNYNAKFKGRVTFTTDTSTSTAYMELRSLRSDDTAVYYCARSGAGKRDSGAFAYWGQGTLVTVSS

SEQ ID NO: 1032 - protein, 111 aa, synthetic construct
AIQLTQSPSSLSASVGDRVTITCRASESIDTKGYSFMHWYQQKPGKAPKLLVYRTSNLESGVPSRFSGSGSGTDFTLTISSLQPEDFATYYCQQSNEDPWTFGGGTKVEIK

SEQ ID NO: 1033 - protein, 122 aa, synthetic construct
QVQLVQSGAEVKKPGASVKVSCKASGYTFTSYWIEWVRQAPGQGLEWIGEILPGEGSTNYNAKFKGRVTFTRDTSTSTAYMELSSLRSEDTAVYYCARSGAGGYDSGAFAYWGQGTLVTVSS

SEQ ID NO: 1034 - protein, 111 aa, synthetic construct; identical to SEQ ID NO: 1020

SEQ ID NO: 1035 - protein, 122 aa, synthetic construct
QVQLVQSGAEVKKPGASVKVSCKASGYTFTSYWIEWVRQAPGQGLEWIGEILPGEGSTNYNAKFKGRVTFTTDTSTSTAYMELRSLRSDDTAVYYCARSGAGGYDSGAFAYWGQGTLVTVSS

SEQ ID NO: 1036 - protein, 111 aa, synthetic construct; identical to SEQ ID NO: 1022

SEQ ID NO: 1037 - protein, 122 aa, synthetic construct
QVQLVQSGAEVKKPGASVKVSCKASGYTFTSYWIEWVRQAPGQGLEWIGEILPGSGSTNYNAKFKGRVTFTRDTSTSTAYMELSSLRSEDTAVYYCARSGAGGYDSGAFAYWGQGTLVTVSS

SEQ ID NO: 1038 - protein, 111 aa, synthetic construct; identical to SEQ ID NO: 1020

SEQ ID NO: 1039 - protein, 122 aa, synthetic construct
QVQLVQSGAEVKKPGASVKVSCKASGYTFTSYWIEWVRQAPGQGLEWIGEILPGSGSTNYNAKFKGRVTFTTDTSTSTAYMELRSLRSDDTAVYYCARSGAGGYDSGAFAYWGQGTLVTVSS

SEQ ID NO: 1040 - protein, 111 aa, synthetic construct; identical to SEQ ID NO: 1022

SEQ ID NO: 1041 - protein, 122 aa, synthetic construct
QVQLVQSGAEVKKPGASVKVSCKASGYTFTSYWIEWVRQAPGQGLEWIGEILPGDESTNYNAKFKGRVTFTRDTSTSTAYMELSSLRSEDTAVYYCARSGAGGYDSGAFAYWGQGTLVTVSS

SEQ ID NO: 1042 - protein, 111 aa, synthetic construct; identical to SEQ ID NO: 1020

SEQ ID NO: 1043 - protein, 122 aa, synthetic construct
QVQLVQSGAEVKKPGASVKVSCKASGYTFTSYWIEWVRQAPGQGLEWIGEILPGDESTNYNAKFKGRVTFTTDTSTSTAYMELRSLRSDDTAVYYCARSGAGGYDSGAFAYWGQGTLVTVSS

SEQ ID NO: 1044 - protein, 111 aa, synthetic construct; identical to SEQ ID NO: 1022

SEQ ID NO: 1045 - protein, 122 aa, synthetic construct
QVQLVQSGAEVKKPGASVKVSCKASGYTFSSYWIEWVRQAPGQGLEWIGEILPGSGSTNYNAKFKGQATFTADTSTSTAYMELSSLRSEDTAVYYCARSGAGGYDSGAFAYWGQGTLVTVSS

SEQ ID NO: 1046 - protein, 111 aa, synthetic construct
EIVLTQSPATLSLSPGERATLSCRASESVDTYGNSFMHWYQQKPGQAPRLLIYRTSNLESGIPARFSGSGSGTDFTLTISSLEPEDFAVYYCQQSNEDPWTFGGGTKVEIK

SEQ ID NO: 1047 - protein, 122 aa, synthetic construct
QVQLVQSGAEVKKPGASVKVSCKASGYTFSSYWIEWVRQAPGQGLEWIGEILPGEGSTNYNAKFKGQATFTADTSTSTAYMELSSLRSEDTAVYYCARSGAGGYDSGAFAYWGQGTLVTVSS

SEQ ID NO: 1048 - protein, 111 aa, synthetic construct; identical to SEQ ID NO: 1046

SEQ ID NO: 1049 - protein, 122 aa, synthetic construct
QVQLVQSGAEVKKPGASVKVSCKASGYTFSSYWIEWVRQAPGQGLEWIGEILPGDESTNYNAKFKGQATFTADTSTSTAYMELSSLRSEDTAVYYCARSGAGGYDSGAFAYWGQGTLVTVSS

SEQ ID NO: 1050 - protein, 111 aa, synthetic construct; identical to SEQ ID NO: 1046

SEQ ID NO: 1051 - protein, 122 aa, synthetic construct; identical to SEQ ID NO: 1045

SEQ ID NO: 1052 - protein, 111 aa, synthetic construct
AIQLTQSPSSLSASVGDRVTITCRASESVDTYGNSFMHWYQQKPGKAPKLLIYRTSNLESGVPSRFSGSGSGTDFTLTISSLQPEDFATYYCQQSNEDPWTFGGGTKVEIK

SEQ ID NO: 1053 - protein, 122 aa, synthetic construct; identical to SEQ ID NO: 1047

SEQ ID NO: 1054 - protein, 111 aa, synthetic construct; identical to SEQ ID NO: 1052

SEQ ID NO: 1055 - protein, 122 aa, synthetic construct; identical to SEQ ID NO: 1049

SEQ ID NO: 1056 - protein, 111 aa, synthetic construct; identical to SEQ ID NO: 1052

SEQ ID NO: 1057 - protein, 214 aa, synthetic construct
DIQMTQSPASLSTFLGEPVTIECRASEDIYNDLAWYQQKPGKSPQLLIYNANSLHTGVPSRFSGSGSGTQYSLKINSLQSEDVASYFCQQYYDYPLTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1058 - protein, 450 aa, Homo sapiens
QVTLKESGPGILQPSHTLSLTCSFSGFSLNTYAMGVSWIRQPSGKGLEWLASIWWNGNKYNNPSLKSRLTVSKDTSNNQAFLKVTSVDTADTATYYCAHSRIIRFTDYVMDAWGQGASVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1059 - protein, 213 aa, synthetic construct
DIQMTQSPSSLSASVGDRVTITCRTSQDISNYLNWYQQKPGKAVKLLISYTSRLHSGVPSRFSGSGSGTDYTLTISSLQPEDFATYYCQQGNTLPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1060 - protein, 447 aa, Homo sapiens
DVQLQGSGPGLVKPSETLSLTCSVTGYSITSGYYWNWIRQFPGKKLEWMGYISYDGSNNYNPSLKNRITISRDTDKNQFSLKLNSVTAADTATYYCAHGYSYYYAMDAWGQGTSSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1061 - protein, 218 aa, synthetic construct
DIQLTQSPSFLSASVGDRVTITCRASESVDYYGNSFMYWYQQKPGKAPKLLIYFASNLESGVPSRFSGSGSGTEFTLTISSLQPEDFATYYCQQNNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1062 - protein, 455 aa, Homo sapiens
QVQLVQSGAEVKKPGASVKVSCKASGYTFTDYYINWVRQAPGQGLEWMGNVNPNDGGTTYNQKFKGRVTMTTDTSTSTAYMELRSLRSDDTAVYYCARREIYFYGTIYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1063 - protein, 8 aa, synthetic construct
GYTFTRYV

SEQ ID NO: 1064 - protein, 8 aa, synthetic construct
INPNNEET

SEQ ID NO: 1065 - protein, 20 aa, synthetic construct
ARADLEILPYYDHARYAMDY

SEQ ID NO: 1066 - protein, 6 aa, synthetic construct
ENIYSH

SEQ ID NO: 1067 - skipped

SEQ ID NO: 1068 - protein, 9 aa, synthetic construct
QHFWSTPPT

SEQ ID NO: 1069 - protein, 5 aa, synthetic construct
RYVMH

SEQ ID NO: 1070 - protein, 17 aa, synthetic construct
WINPNNEETKYSQKFKG

SEQ ID NO: 1071 - protein, 18 aa, synthetic construct
ADLEILPYYDHARYAMDY

SEQ ID NO: 1072 - protein, 11 aa, synthetic construct
RASENIYSHLA

SEQ ID NO: 1073 - protein, 7 aa, synthetic construct
NAKSLQS

SEQ ID NO: 1074 - protein, 9 aa, synthetic construct
QHFWSTPPT

SEQ ID NO: 1075 - protein, 7 aa, synthetic construct
GYTFTRY

SEQ ID NO: 1076 - protein, 6 aa, synthetic construct
NPNNEE

SEQ ID NO: 1077 - protein, 18 aa, synthetic construct
ADLEILPYYDHARYAMDY

SEQ ID NO: 1078 - protein, 11 aa, synthetic construct
RASENIYSHLA

SEQ ID NO: 1079 - protein, 7 aa, synthetic construct
NAKSLQS

SEQ ID NO: 1080 - protein, 9 aa, synthetic construct
QHFWSTPPT

SEQ ID NO: 1081 - protein, 13 aa, synthetic construct
KASGYTFTRYVMH

SEQ ID NO: 1082 - protein, 10 aa, synthetic construct
WINPNNEETK

SEQ ID NO: 1083 - protein, 20 aa, synthetic construct
ARADLEILPYYDHARYAMDY

SEQ ID NO: 1084 - protein, 11 aa, synthetic construct
RASENIYSHLA

SEQ ID NO: 1085 - protein, 8 aa, synthetic construct
YNAKSLQS

SEQ ID NO: 1086 - protein, 9 aa, synthetic construct
QHFWSTPPT

SEQ ID NO: 1087 - protein, 8 aa, synthetic construct
GYTFTRYV

SEQ ID NO: 1088 - protein, 8 aa, synthetic construct
INPNNEET

SEQ ID NO: 1089 - protein, 20 aa, synthetic construct
ARADLEILPYYDHARYAMDY

SEQ ID NO: 1090 - protein, 6 aa, synthetic construct
ENIYSH

SEQ ID NO: 1091 - skipped

SEQ ID NO: 1092 - protein, 9 aa, synthetic construct
QHFWSTPPT

SEQ ID NO: 1093 - protein, 5 aa, synthetic construct
RYVMH

SEQ ID NO: 1094 - protein, 17 aa, synthetic construct
YINPNNEETKYSQKFKG

SEQ ID NO: 1095 - protein, 18 aa, synthetic construct
ADLEILPYYDHARYAMDY

SEQ ID NO: 1096 - protein, 11 aa, synthetic construct
RASENIYSHLA

SEQ ID NO: 1097 - protein, 7 aa, synthetic construct
NAKSLQS

SEQ ID NO: 1098 - protein, 9 aa, synthetic construct
QHFWSTPPT

SEQ ID NO: 1099 - protein, 7 aa, synthetic construct
GYTFTRY

SEQ ID NO: 1100 - protein, 6 aa, synthetic construct
NPNNEE

SEQ ID NO: 1101 - protein, 21 aa, synthetic construct
ADLEILPYYDHARYAMDYSEQ

SEQ ID NO: 1102 - protein, 11 aa, synthetic construct
RASENIYSHLA

SEQ ID NO: 1103 - protein, 7 aa, synthetic construct
NAKSLQS

SEQ ID NO: 1104 - protein, 9 aa, synthetic construct
QHFWSTPPT

SEQ ID NO: 1105 - protein, 13 aa, synthetic construct
KASGYTFTRYVMH

SEQ ID NO: 1106 - protein, 10 aa, synthetic construct
YINPNNEETK

SEQ ID NO: 1107 - protein, 20 aa, synthetic construct
ARADLEILPYYDHARYAMDY

SEQ ID NO: 1108 - protein, 11 aa, synthetic construct
RASENIYSHLA

SEQ ID NO: 1109 - protein, 8 aa, synthetic construct
YNAKSLQS

SEQ ID NO: 1110 - protein, 9 aa, synthetic construct
QHFWSTPPT

SEQ ID NO: 1111 - protein, 8 aa, synthetic construct
GYTFTRYV

SEQ ID NO: 1112 - protein, 8 aa, synthetic construct
INPNNEET

SEQ ID NO: 1113 - protein, 20 aa, synthetic construct
ARADLEILPYYDHARYAMDY

SEQ ID NO: 1114 - protein, 6 aa, synthetic construct
ENIYSH

SEQ ID NO: 1115 - skipped

SEQ ID NO: 1116 - protein, 9 aa, synthetic construct
QHFWSTPPT

SEQ ID NO: 1117 - protein, 5 aa, synthetic construct
RYVMH

SEQ ID NO: 1118 - protein, 17 aa, synthetic construct
YINPNNEETKYNEKFKG

SEQ ID NO: 1119 - protein, 18 aa, synthetic construct
ADLEILPYYDHARYAMDY

SEQ ID NO: 1120 - protein, 11 aa, synthetic construct
RASENIYSHLA

SEQ ID NO: 1121 - protein, 7 aa, synthetic construct
NAKSLQS

SEQ ID NO: 1122 - protein, 9 aa, synthetic construct
QHFWSTPPT

SEQ ID NO: 1123 - protein, 7 aa, synthetic construct
GYTFTRY

SEQ ID NO: 1124 - protein, 6 aa, synthetic construct
NPNNEE

SEQ ID NO: 1125 - protein, 18 aa, synthetic construct
ADLEILPYYDHARYAMDY

SEQ ID NO: 1126 - protein, 11 aa, synthetic construct
RASENIYSHLA

SEQ ID NO: 1127 - protein, 7 aa, synthetic construct
NAKSLQS

SEQ ID NO: 1128 - protein, 9 aa, synthetic construct
QHFWSTPPT

SEQ ID NO: 1129 - protein, 13 aa, synthetic construct
KASGYTFTRYVMH

SEQ ID NO: 1130 - protein, 10 aa, synthetic construct
YINPNNEETK

SEQ ID NO: 1131 - protein, 20 aa, synthetic construct
ARADLEILPYYDHARYAMDY

SEQ ID NO: 1132 - protein, 11 aa, synthetic construct
RASENIYSHLA

SEQ ID NO: 1133 - protein, 8 aa, synthetic construct
YNAKSLQS

SEQ ID NO: 1134 - protein, 9 aa, synthetic construct
QHFWSTPPT

SEQ ID NO: 1135 - protein, 8 aa, synthetic construct
GYTFTRYV

SEQ ID NO: 1136 - protein, 8 aa, synthetic construct
INPNNEET

SEQ ID NO: 1137 - protein, 20 aa, synthetic construct
ARADLEILPYYDHARYAMDY

SEQ ID NO: 1138 - protein, 6 aa, synthetic construct
GNIHNY

SEQ ID NO: 1139 - skipped

SEQ ID NO: 1140 - protein, 9 aa, synthetic construct
QHFWSSPPT

SEQ ID NO: 1141 - protein, 5 aa, synthetic construct
RYVMH

SEQ ID NO: 1142 - protein, 17 aa, synthetic construct
WINPNNEETKYSQKFKG

SEQ ID NO: 1143 - protein, 18 aa, synthetic construct
ADLEILPYYDHARYAMDY

SEQ ID NO: 1144 - protein, 11 aa, synthetic construct
RASGNIHNYLA

SEQ ID NO: 1145 - protein, 7 aa, synthetic construct
NAKSLQS

SEQ ID NO: 1146 - protein, 9 aa, synthetic construct
QHFWSSPPT

SEQ ID NO: 1147 - protein, 7 aa, synthetic construct
GYTFTRY

SEQ ID NO: 1148 - protein, 6 aa, synthetic construct
NPNNEE

SEQ ID NO: 1149 - protein, 18 aa, synthetic construct
ADLEILPYYDHARYAMDY

SEQ ID NO: 1150 - protein, 11 aa, synthetic construct
RASGNIHNYLA

SEQ ID NO: 1151 - protein, 7 aa, synthetic construct
NAKSLQS

SEQ ID NO: 1152 - protein, 9 aa, synthetic construct
QHFWSSPPT

SEQ ID NO: 1153 - protein, 13 aa, synthetic construct
KASGYTFTRYVMH

SEQ ID NO: 1154 - protein, 10 aa, synthetic construct
WINPNNEETK

SEQ ID NO: 1155 - protein, 20 aa, synthetic construct
ARADLEILPYYDHARYAMDY

SEQ ID NO: 1156 - protein, 11 aa, synthetic construct
RASGNIHNYLA

SEQ ID NO: 1157 - protein, 8 aa, synthetic construct
YNAKSLQS

SEQ ID NO: 1158 - protein, 9 aa, synthetic construct
QHFWSSPPT

SEQ ID NO: 1159 - protein, 8 aa, synthetic construct
GYTFTRYV

SEQ ID NO: 1160 - protein, 8 aa, synthetic construct
INPNNEET

SEQ ID NO: 1161 - protein, 20 aa, synthetic construct
ARADLEILPYYDHARYAMDY

SEQ ID NO: 1162 - protein, 6 aa, synthetic construct
GNIHNY

SEQ ID NO: 1163 - skipped

SEQ ID NO: 1164 - protein, 9 aa, synthetic construct
QHFWSTPPT

SEQ ID NO: 1165 - protein, 5 aa, synthetic construct
RYVMH

SEQ ID NO: 1166 - protein, 17 aa, synthetic construct
WINPNNEETKYSQKFKG

SEQ ID NO: 1167 - protein, 18 aa, synthetic construct
ADLEILPYYDHARYAMDY

SEQ ID NO: 1168 - protein, 11 aa, synthetic construct
RASGNIHNYLA

SEQ ID NO: 1169 - protein, 7 aa, synthetic construct
NAKSLQS

SEQ ID NO: 1170 - protein, 9 aa, synthetic construct
QHFWSTPPT

SEQ ID NO: 1171 - protein, 7 aa, synthetic construct
GYTFTRY

SEQ ID NO: 1172 - protein, 6 aa, synthetic construct
NPNNEE

SEQ ID NO: 1173 - protein, 18 aa, synthetic construct
ADLEILPYYDHARYAMDY

SEQ ID NO: 1174 - protein, 11 aa, synthetic construct
RASGNIHNYLA

SEQ ID NO: 1175 - protein, 7 aa, synthetic construct
NAKSLQS

SEQ ID NO: 1176 - protein, 9 aa, synthetic construct
QHFWSTPPT

SEQ ID NO: 1177 - protein, 13 aa, synthetic construct
KASGYTFTRYVMH

SEQ ID NO: 1178 - protein, 10 aa, synthetic construct
WINPNNEETK

SEQ ID NO: 1179 - protein, 20 aa, synthetic construct
ARADLEILPYYDHARYAMDY

SEQ ID NO: 1180 - protein, 11 aa, synthetic construct
RASGNIHNYLA

SEQ ID NO: 1181 - protein, 8 aa, synthetic construct
YNAKSLQS

SEQ ID NO: 1182 - protein, 9 aa, synthetic construct
QHFWSTPPT

SEQ ID NO: 1183 - protein, 8 aa, synthetic construct
GYTFTRYV

SEQ ID NO: 1184 - protein, 8 aa, synthetic construct
INPNNEET

SEQ ID NO: 1185 - protein, 20 aa, synthetic construct
ARADLEILPYYDHARYAMDY

SEQ ID NO: 1186 - protein, 6 aa, synthetic construct
GNIHNY

SEQ ID NO: 1187 - skipped

SEQ ID NO: 1188 - protein, 9 aa, synthetic construct
QHFWSSPPT

SEQ ID NO: 1189 - protein, 5 aa, synthetic construct
RYVMH

SEQ ID NO: 1190 - protein, 17 aa, synthetic construct
WINPNNEETKYSQKFKG

SEQ ID NO: 1191 - protein, 18 aa, synthetic construct
ADLEILPYYDHARYAMDY

SEQ ID NO: 1192 - protein, 11 aa, synthetic construct
RASGNIHNYLA

SEQ ID NO: 1193 - protein, 7 aa, synthetic construct
NAKTLAE

SEQ ID NO: 1194 - protein, 9 aa, synthetic construct
QHFWSSPPT

SEQ ID NO: 1195 - protein, 7 aa, synthetic construct
GYTFTRY

SEQ ID NO: 1196 - protein, 6 aa, synthetic construct
NPNNEE

SEQ ID NO: 1197 - protein, 18 aa, synthetic construct
ADLEILPYYDHARYAMDY

SEQ ID NO: 1198 - protein, 11 aa, synthetic construct
RASGNIHNYLA

SEQ ID NO: 1199 - protein, 7 aa, synthetic construct
NAKTLAE

SEQ ID NO: 1200 - protein, 9 aa, synthetic construct
QHFWSSPPT

SEQ ID NO: 1201 - protein, 13 aa, synthetic construct
KASGYTFTRYVMH

SEQ ID NO: 1202 - protein, 10 aa, synthetic construct
WINPNNEETK

SEQ ID NO: 1203 - protein, 20 aa, synthetic construct
ARADLEILPYYDHARYAMDY

SEQ ID NO: 1204 - protein, 11 aa, synthetic construct
RASGNIHNYLA

SEQ ID NO: 1205 - protein, 8 aa, synthetic construct
YNAKTLAE

SEQ ID NO: 1206 - protein, 9 aa, synthetic construct
QHFWSSPPT

SEQ ID NO: 1207 - protein, 8 aa, synthetic construct
GYTFTRYV

SEQ ID NO: 1208 - protein, 8 aa, synthetic construct
INPNNEET

SEQ ID NO: 1209 - protein, 20 aa, synthetic construct
ARADLEILPYYDHARYAMDY

SEQ ID NO: 1210 - protein, 6 aa, synthetic construct
GNIHNY

SEQ ID NO: 1211 - skipped

SEQ ID NO: 1212 - protein, 9 aa, synthetic construct
QHFWSSPPT

SEQ ID NO: 1213 - protein, 5 aa, synthetic construct
RYVMH

SEQ ID NO: 1214 - protein, 17 aa, synthetic construct
WINPNNEETKYSQKFKG

SEQ ID NO: 1215 - protein, 18 aa, synthetic construct
ADLEILPYYDHARYAMDY

SEQ ID NO: 1216 - protein, 11 aa, synthetic construct
RASGNIHNYLA

SEQ ID NO: 1217 - protein, 7 aa, synthetic construct
NAKTLAS

SEQ ID NO: 1218 - protein, 9 aa, synthetic construct
QHFWSSPPT

SEQ ID NO: 1219 - protein, 7 aa, synthetic construct
GYTFTRY

SEQ ID NO: 1220 - protein, 6 aa, synthetic construct
NPNNEE

SEQ ID NO: 1221 - protein, 18 aa, synthetic construct
ADLEILPYYDHARYAMDY

SEQ ID NO: 1222 - protein, 11 aa, synthetic construct
RASGNIHNYLA

SEQ ID NO: 1223 - protein, 7 aa, synthetic construct
NAKTLAS

SEQ ID NO: 1224 - protein, 9 aa, synthetic construct
QHFWSSPPT

SEQ ID NO: 1225 - protein, 13 aa, synthetic construct
KASGYTFTRYVMH

SEQ ID NO: 1226 - protein, 10 aa, synthetic construct
WINPNNEETK

SEQ ID NO: 1227 - protein, 20 aa, synthetic construct
ARADLEILPYYDHARYAMDY

SEQ ID NO: 1228 - protein, 11 aa, synthetic construct
RASGNIHNYLA

SEQ ID NO: 1229 - protein, 8 aa, synthetic construct
YNAKTLAS

SEQ ID NO: 1230 - protein, 9 aa, synthetic construct
QHFWSSPPT

SEQ ID NO: 1231 - protein, 8 aa, synthetic construct
GYTFTSYW

SEQ ID NO: 1232 - protein, 8 aa, synthetic construct
ILPGDGST

SEQ ID NO: 1233 - protein, 15 aa, synthetic construct
ARSGAGGYDSGAFAY

SEQ ID NO: 1234 - protein, 10 aa, synthetic construct
ESVDTYGNSF

SEQ ID NO: 1235 - skipped

SEQ ID NO: 1236 - protein, 9 aa, synthetic construct
QQSNEDPWT

SEQ ID NO: 1237 - protein, 5 aa, synthetic construct
SYWIE

SEQ ID NO: 1238 - protein, 17 aa, synthetic construct
EILPGDGSTNYNAKFKG

SEQ ID NO: 1239 - protein, 13 aa, synthetic construct
SGAGGYDSGAFAY

SEQ ID NO: 1240 - protein, 15 aa, synthetic construct
RASESVDTYGNSFMH

SEQ ID NO: 1241 - protein, 7 aa, synthetic construct
RTSNLES

SEQ ID NO: 1242 - protein, 9 aa, synthetic construct
QQSNEDPWT

SEQ ID NO: 1243 - protein, 7 aa, synthetic construct
GYTFTSY

SEQ ID NO: 1244 - protein, 6 aa, synthetic construct
LPGDGS

SEQ ID NO: 1245 - protein, 13 aa, synthetic construct
SGAGGYDSGAFAY

SEQ ID NO: 1246 - protein, 15 aa, synthetic construct
RASESVDTYGNSFMH

SEQ ID NO: 1247 - protein, 7 aa, synthetic construct
RTSNLES

SEQ ID NO: 1248 - protein, 9 aa, synthetic construct
QQSNEDPWT

SEQ ID NO: 1249 - protein, 13 aa, synthetic construct
KASGYTFTSYWIE

SEQ ID NO: 1250 - protein, 10 aa, synthetic construct
EILPGDGSTN

SEQ ID NO: 1251 - protein, 15 aa, synthetic construct
ARSGAGGYDSGAFAY

SEQ ID NO: 1252 - protein, 15 aa, synthetic construct
RASESVDTYGNSFMH

SEQ ID NO: 1253 - protein, 8 aa, synthetic construct
YRTSNLES

SEQ ID NO: 1254 - protein, 9 aa, synthetic construct
QQSNEDPWT

SEQ ID NO: 1255 - protein, 8 aa, synthetic construct
GYTFTSYW

SEQ ID NO: 1256 - protein, 8 aa, synthetic construct
ILPGDGST

SEQ ID NO: 1257 - protein, 15 aa, synthetic construct
ARSGAGGYDSGAFAY

SEQ ID NO: 1258 - protein, 10 aa, synthetic construct
ESVDTYGNSF

SEQ ID NO: 1259 - skipped

SEQ ID NO: 1260 - protein, 9 aa, synthetic construct
QQSNEDPWT

SEQ ID NO: 1261 - protein, 5 aa, synthetic construct
SYWIE

SEQ ID NO: 1262 - protein, 17 aa, synthetic construct
EILPGDGSTNYNAKFKG

SEQ ID NO: 1263 - protein, 13 aa, synthetic construct
SGAGGYDSGAFAY

SEQ ID NO: 1264 - protein, 15 aa, synthetic construct
RASESVDTYGNSFMH

SEQ ID NO: 1265 - protein, 7 aa, synthetic construct
RTSNLES

SEQ ID NO: 1266 - protein, 9 aa, synthetic construct
QQSNEDPWT

SEQ ID NO: 1267 - protein, 7 aa, synthetic construct
GYTFTSY

SEQ ID NO: 1268 - protein, 6 aa, synthetic construct
LPGDGS

SEQ ID NO: 1269 - protein, 13 aa, synthetic construct
SGAGGYDSGAFAY

SEQ ID NO: 1270 - protein, 15 aa, synthetic construct
RASESVDTYGNSFMH

SEQ ID NO: 1271 - protein, 7 aa, synthetic construct
RTSNLES

SEQ ID NO: 1272 - protein, 9 aa, synthetic construct
QQSNEDPWT

SEQ ID NO: 1273 - protein, 13 aa, synthetic construct
KASGYTFTSYWIE

SEQ ID NO: 1274 - protein, 10 aa, synthetic construct
EILPGDGSTN

SEQ ID NO: 1275 - protein, 15 aa, synthetic construct
ARSGAGGYDSGAFAY

SEQ ID NO: 1276 - protein, 15 aa, synthetic construct
RASESVDTYGNSFMH

SEQ ID NO: 1277 - protein, 8 aa, synthetic construct
YRTSNLES

SEQ ID NO: 1278 - protein, 9 aa, synthetic construct
QQSNEDPWT

SEQ ID NO: 1279 - protein, 8 aa, synthetic construct
GYTFTSYW

SEQ ID NO: 1280 - protein, 8 aa, synthetic construct
ILPGSGST

SEQ ID NO: 1281 - protein, 15 aa, synthetic construct
ARSGPGKRDSGGFAY

SEQ ID NO: 1282 - protein, 10 aa, synthetic construct
ESIDSKGYSF

SEQ ID NO: 1283 - skipped

SEQ ID NO: 1284 - protein, 9 aa, synthetic construct
QQSNEDPWT

SEQ ID NO: 1285 - protein, 5 aa, synthetic construct
SYWIE

SEQ ID NO: 1286 - protein, 17 aa, synthetic construct
EILPGSGSTNYNAKFKG

SEQ ID NO: 1287 - protein, 13 aa, synthetic construct
SGPGKRDSGGFAY

SEQ ID NO: 1288 - protein, 15 aa, synthetic construct
RASESIDSKGYSFMH

SEQ ID NO: 1289 - protein, 7 aa, synthetic construct
RTSNLES

SEQ ID NO: 1290 - protein, 9 aa, synthetic construct
QQSNEDPWT

SEQ ID NO: 1291 - protein, 7 aa, synthetic construct
GYTFTSY

SEQ ID NO: 1292 - protein, 6 aa, synthetic construct
LPGSGS

SEQ ID NO: 1293 - protein, 13 aa, synthetic construct
SGPGKRDSGGFAY

SEQ ID NO: 1294 - protein, 15 aa, synthetic construct
RASESIDSKGYSFMH

SEQ ID NO: 1295 - protein, 7 aa, synthetic construct
RTSNLES

SEQ ID NO: 1296 - protein, 9 aa, synthetic construct
QQSNEDPWT

SEQ ID NO: 1297 - protein, 13 aa, synthetic construct
KASGYTFTSYWIE

SEQ ID NO: 1298 - protein, 10 aa, synthetic construct
EILPGSGSTN

SEQ ID NO: 1299 - protein, 15 aa, synthetic construct
ARSGPGKRDSGGFAY

SEQ ID NO: 1300 - protein, 15 aa, synthetic construct
RASESIDSKGYSFMH

SEQ ID NO: 1301 - protein, 8 aa, synthetic construct
YRTSNLES

SEQ ID NO: 1302 - protein, 9 aa, synthetic construct
QQSNEDPWT

SEQ ID NO: 1303 - protein, 8 aa, synthetic construct
GYTFTSYW

SEQ ID NO: 1304 - protein, 8 aa, synthetic construct
ILPGSGST

SEQ ID NO: 1305 - protein, 15 aa, synthetic construct
ARSGPGKRDSGGFAY

SEQ ID NO: 1306 - protein, 10 aa, synthetic construct
ESIDSKGYSF

SEQ ID NO: 1307 - skipped

SEQ ID NO: 1308 - protein, 9 aa, synthetic construct
QQSNEDPWT

SEQ ID NO: 1309 - protein, 5 aa, synthetic construct
SYWIE

SEQ ID NO: 1310 - protein, 17 aa, synthetic construct
EILPGSGSTNYNAKFKG

SEQ ID NO: 1311 - protein, 13 aa, synthetic construct
SGPGKRDSGGFAY

SEQ ID NO: 1312 - protein, 15 aa, synthetic construct
RASESIDSKGYSFMH

SEQ ID NO: 1313 - protein, 7 aa, synthetic construct
RTSNLES

SEQ ID NO: 1314 - protein, 9 aa, synthetic construct
QQSNEDPWT

SEQ ID NO: 1315 - protein, 7 aa, synthetic construct
GYTFTSY

SEQ ID NO: 1316 - protein, 6 aa, synthetic construct
LPGSGS

SEQ ID NO: 1317 - protein, 13 aa, synthetic construct
SGPGKRDSGGFAY

SEQ ID NO: 1318 - protein, 15 aa, synthetic construct
RASESIDSKGYSFMH

SEQ ID NO: 1319 - protein, 7 aa, synthetic construct
RTSNLES

SEQ ID NO: 1320 - protein, 9 aa, synthetic construct
QQSNEDPWT

SEQ ID NO: 1321 - protein, 13 aa, synthetic construct
KASGYTFTSYWIE

SEQ ID NO: 1322 - protein, 10 aa, synthetic construct
EILPGSGSTN

SEQ ID NO: 1323 - protein, 15 aa, synthetic construct
ARSGPGKRDSGGFAY

SEQ ID NO: 1324 - protein, 15 aa, synthetic construct
RASESIDSKGYSFMH

SEQ ID NO: 1325 - protein, 8 aa, synthetic construct
YRTSNLES

SEQ ID NO: 1326 - protein, 9 aa, synthetic construct
QQSNEDPWT

SEQ ID NO: 1327 - protein, 8 aa, synthetic construct
GYTFTNYW

SEQ ID NO: 1328 - protein, 8 aa, synthetic construct
ILPGEGST

SEQ ID NO: 1329 - protein, 15 aa, synthetic construct
ARSGRGGYESGAFAY

SEQ ID NO: 1330 - protein, 10 aa, synthetic construct
ESVDTEGYRF

SEQ ID NO: 1331 - skipped

SEQ ID NO: 1332 - protein, 9 aa, synthetic construct
QQSNEDPWT

SEQ ID NO: 1333 - protein, 5 aa, synthetic construct
NYWIE

SEQ ID NO: 1334 - protein, 17 aa, synthetic construct
EILPGEGSTNYNAKFKG

SEQ ID NO: 1335 - protein, 13 aa, synthetic construct
SGRGGYESGAFAY

SEQ ID NO: 1336 - protein, 15 aa, synthetic construct
RASESVDTEGYRFMH

SEQ ID NO: 1337 - protein, 7 aa, synthetic construct
RTSNLES

SEQ ID NO: 1338 - protein, 9 aa, synthetic construct
QQSNEDPWT

SEQ ID NO: 1339 - protein, 7 aa, synthetic construct
GYTFTNY

SEQ ID NO: 1340 - protein, 6 aa, synthetic construct
LPGEGS

SEQ ID NO: 1341 - protein, 13 aa, synthetic construct
SGRGGYESGAFAY

SEQ ID NO: 1342 - protein, 15 aa, synthetic construct
RASESVDTEGYRFMH

SEQ ID NO: 1343 - protein, 7 aa, synthetic construct
RTSNLES

SEQ ID NO: 1344 - protein, 9 aa, synthetic construct
QQSNEDPWT

SEQ ID NO: 1345 - protein, 13 aa, synthetic construct
KASGYTFTNYWIE

SEQ ID NO: 1346 - protein, 10 aa, synthetic construct
EILPGEGSTN

SEQ ID NO: 1347 - protein, 15 aa, synthetic construct
ARSGRGGYESGAFAY

SEQ ID NO: 1348 - protein, 15 aa, synthetic construct
RASESVDTEGYRFMH

SEQ ID NO: 1349 - protein, 8 aa, synthetic construct
YRTSNLES

SEQ ID NO: 1350 - protein, 9 aa, synthetic construct
QQSNEDPWT

SEQ ID NO: 1351 - protein, 8 aa, synthetic construct
GYTFSNSW

SEQ ID NO: 1352 - protein, 8 aa, synthetic construct
ILPGEGST

SEQ ID NO: 1353 - protein, 15 aa, synthetic construct
ARSGRGHYDSGRFAY

SEQ ID NO: 1354 - protein, 10 aa, synthetic construct
ESVDSHGYRF

SEQ ID NO: 1355 - skipped

SEQ ID NO: 1356 - protein, 9 aa, synthetic construct
QQSNEDPWT

SEQ ID NO: 1357 - protein, 5 aa, synthetic construct
NSWIE

SEQ ID NO: 1358 - protein, 17 aa, synthetic construct
EILPGEGSTNYNAKFKG

SEQ ID NO: 1359 - protein, 13 aa, synthetic construct
SGRGHYDSGRFAY

SEQ ID NO: 1360 - protein, 15 aa, synthetic construct
RASESVDSHGYRFMH

SEQ ID NO: 1361 - protein, 7 aa, synthetic construct
RTSNLES

SEQ ID NO: 1362 - protein, 9 aa, synthetic construct
QQSNEDPWT

SEQ ID NO: 1363 - protein, 7 aa, synthetic construct
GYTFSNS

SEQ ID NO: 1364 - protein, 6 aa, synthetic construct
LPGEGS

SEQ ID NO: 1365 - protein, 13 aa, synthetic construct
SGRGHYDSGRFAY

SEQ ID NO: 1366 - protein, 15 aa, synthetic construct
RASESVDSHGYRFMH

SEQ ID NO: 1367 - protein, 7 aa, synthetic construct
RTSNLES

SEQ ID NO: 1368 - protein, 9 aa, synthetic construct
QQSNEDPWT

SEQ ID NO: 1369 - protein, 13 aa, synthetic construct
KASGYTFSNSWIE

SEQ ID NO: 1370 - protein, 10 aa, synthetic construct
EILPGEGSTN

SEQ ID NO: 1371 - protein, 15 aa, synthetic construct
ARSGRGHYDSGRFAY

SEQ ID NO: 1372 - protein, 15 aa, synthetic construct
RASESVDSHGYRFMH

SEQ ID NO: 1373 - protein, 8 aa, synthetic construct
YRTSNLES

SEQ ID NO: 1374 - protein, 9 aa, synthetic construct
QQSNEDPWT

SEQ ID NO: 1375 - protein, 8 aa, synthetic construct
GYTFTSYW

SEQ ID NO: 1376 - protein, 8 aa, synthetic construct
ILPGSGST

SEQ ID NO: 1377 - protein, 15 aa, synthetic construct
ARSGAGKRDSGAFAY

SEQ ID NO: 1378 - protein, 10 aa, synthetic construct
ESIDTKGYSF

SEQ ID NO: 1379 - skipped

SEQ ID NO: 1380 - protein, 9 aa, synthetic construct
QQSNEDPWT

SEQ ID NO: 1381 - protein, 5 aa, synthetic construct
SYWIE

SEQ ID NO: 1382 - protein, 17 aa, synthetic construct
EILPGSGSTNYNAKFKG

SEQ ID NO: 1383 - protein, 13 aa, synthetic construct
SGAGKRDSGAFAY

SEQ ID NO: 1384 - protein, 15 aa, synthetic construct
RASESIDTKGYSFMH

SEQ ID NO: 1385 - protein, 7 aa, synthetic construct
RTSNLES

SEQ ID NO: 1386 - protein, 9 aa, synthetic construct
QQSNEDPWT

SEQ ID NO: 1387 - protein, 7 aa, synthetic construct
GYTFTSY

SEQ ID NO: 1388 - protein, 6 aa, synthetic construct
LPGSGS

SEQ ID NO: 1389 - protein, 13 aa, synthetic construct
SGAGKRDSGAFAY

SEQ ID NO: 1390 - protein, 15 aa, synthetic construct
RASESIDTKGYSFMH

SEQ ID NO: 1391 - protein, 7 aa, synthetic construct
RTSNLES

SEQ ID NO: 1392 - protein, 9 aa, synthetic construct
QQSNEDPWT

SEQ ID NO: 1393 - protein, 13 aa, synthetic construct
KASGYTFTSYWIE

SEQ ID NO: 1394 - protein, 10 aa, synthetic construct
EILPGSGSTN

SEQ ID NO: 1395 - protein, 15 aa, synthetic construct
ARSGAGKRDSGAFAY

SEQ ID NO: 1396 - protein, 15 aa, synthetic construct
RASESIDTKGYSFMH

SEQ ID NO: 1397 - protein, 8 aa, synthetic construct
YRTSNLES

SEQ ID NO: 1398 - protein, 9 aa, synthetic construct
QQSNEDPWT

SEQ ID NO: 1399 - protein, 8 aa, synthetic construct
GYTFTSYW

SEQ ID NO: 1400 - protein, 8 aa, synthetic construct
ILPGEGST

SEQ ID NO: 1401 - protein, 15 aa, synthetic construct
ARSGAGGYDSGAFAY

SEQ ID NO: 1402 - protein, 10 aa, synthetic construct
ESVDTYGNSF

SEQ ID NO: 1403 - skipped

SEQ ID NO: 1404 - protein, 9 aa, synthetic construct
QQSNEDPWT

SEQ ID NO: 1405 - protein, 5 aa, synthetic construct
SYWIE

SEQ ID NO: 1406 - protein, 17 aa, synthetic construct
EILPGEGSTNYNAKFKG

SEQ ID NO: 1407 - protein, 13 aa, synthetic construct
SGAGGYDSGAFAY

SEQ ID NO: 1408 - protein, 15 aa, synthetic construct
RASESVDTYGNSFMH

SEQ ID NO: 1409 - protein, 7 aa, synthetic construct
RTSNLES

SEQ ID NO: 1410 - protein, 9 aa, synthetic construct
QQSNEDPWT

SEQ ID NO: 1411 - protein, 7 aa, synthetic construct
GYTFTSY

SEQ ID NO: 1412 - protein, 6 aa, synthetic construct
LPGEGS

SEQ ID NO: 1413 - protein, 13 aa, synthetic construct
SGAGGYDSGAFAY

SEQ ID NO: 1414 - protein, 15 aa, synthetic construct
RASESVDTYGNSFMH

SEQ ID NO: 1415 - protein, 7 aa, synthetic construct
RTSNLES

SEQ ID NO: 1416 - protein, 9 aa, synthetic construct
QQSNEDPWT

SEQ ID NO: 1417 - protein, 13 aa, synthetic construct
KASGYTFTSYWIE

SEQ ID NO: 1418 - protein, 10 aa, synthetic construct
EILPGEGSTN

SEQ ID NO: 1419 - protein, 15 aa, synthetic construct
ARSGAGGYDSGAFAY

SEQ ID NO: 1420 - protein, 15 aa, synthetic construct
RASESVDTYGNSFMH

SEQ ID NO: 1421 - protein, 8 aa, synthetic construct
YRTSNLES

SEQ ID NO: 1422 - protein, 9 aa, synthetic construct
QQSNEDPWT

SEQ ID NO: 1423 - protein, 8 aa, synthetic construct
GYTFTSYW

SEQ ID NO: 1424 - protein, 8 aa, synthetic construct
ILPGEGST

SEQ ID NO: 1425 - protein, 15 aa, synthetic construct
ARSGAGGYDSGAFAY

SEQ ID NO: 1426 - protein, 10 aa, synthetic construct
ESVDTYGNSF

SEQ ID NO: 1427 - skipped

SEQ ID NO: 1428 - protein, 9 aa, synthetic construct
QQSNEDPWT

SEQ ID NO: 1429 - protein, 5 aa, synthetic construct
SYWIE

SEQ ID NO: 1430 - protein, 17 aa, synthetic construct
EILPGEGSTNYNAKFKG

SEQ ID NO: 1431 - protein, 13 aa, synthetic construct
SGAGGYDSGAFAY

SEQ ID NO: 1432 - protein, 15 aa, synthetic construct
RASESVDTYGNSFMH

SEQ ID NO: 1433 - protein, 7 aa, synthetic construct
RTSNLES

SEQ ID NO: 1434 - protein, 9 aa, synthetic construct
QQSNEDPWT

SEQ ID NO: 1435 - protein, 7 aa, synthetic construct
GYTFTSY

SEQ ID NO: 1436 - protein, 6 aa, synthetic construct
LPGEGS

SEQ ID NO: 1437 - protein, 13 aa, synthetic construct
SGAGGYDSGAFAY

SEQ ID NO: 1438 - protein, 15 aa, synthetic construct
RASESVDTYGNSFMH

SEQ ID NO: 1439 - protein, 7 aa, synthetic construct
RTSNLES

SEQ ID NO: 1440 - protein, 9 aa, synthetic construct
QQSNEDPWT

SEQ ID NO: 1441 - protein, 13 aa, synthetic construct
KASGYTFTSYWIE

SEQ ID NO: 1442 - protein, 10 aa, synthetic construct
EILPGEGSTN

SEQ ID NO: 1443 - protein, 15 aa, synthetic construct
ARSGAGGYDSGAFAY

SEQ ID NO: 1444 - protein, 15 aa, synthetic construct
RASESVDTYGNSFMH

SEQ ID NO: 1445 - protein, 8 aa, synthetic construct
YRTSNLES

SEQ ID NO: 1446 - protein, 9 aa, synthetic construct
QQSNEDPWT

SEQ ID NO: 1447 - protein, 8 aa, synthetic construct
GYTFTSYW

SEQ ID NO: 1448 - protein, 8 aa, synthetic construct
ILPGSGST

SEQ ID NO: 1449 - protein, 15 aa, synthetic construct
ARSGAGGYDSGAFAY

SEQ ID NO: 1450 - protein, 10 aa, synthetic construct
ESVDTYGNSF

SEQ ID NO: 1451 - skipped

SEQ ID NO: 1452 - protein, 9 aa, synthetic construct
QQSNEDPWT

SEQ ID NO: 1453 - protein, 5 aa, synthetic construct
SYWIE

SEQ ID NO: 1454 - protein, 17 aa, synthetic construct
EILPGSGSTNYNAKFKG

SEQ ID NO: 1455 - protein, 13 aa, synthetic construct
SGAGGYDSGAFAY

SEQ ID NO: 1456 - protein, 15 aa, synthetic construct
RASESVDTYGNSFMH

SEQ ID NO: 1457 - protein, 7 aa, synthetic construct
RTSNLES

SEQ ID NO: 1458 - protein, 9 aa, synthetic construct
QQSNEDPWT

SEQ ID NO: 1459 - protein, 7 aa, synthetic construct
GYTFTSY

SEQ ID NO: 1460 - protein, 6 aa, synthetic construct
LPGSGS

SEQ ID NO: 1461 - protein, 13 aa, synthetic construct
SGAGGYDSGAFAY

SEQ ID NO: 1462 - protein, 15 aa, synthetic construct
RASESVDTYGNSFMH

SEQ ID NO: 1463 - protein, 7 aa, synthetic construct
RTSNLES

SEQ ID NO: 1464 - protein, 9 aa, synthetic construct
QQSNEDPWT

SEQ ID NO: 1465 - protein, 13 aa, synthetic construct
KASGYTFTSYWIE

SEQ ID NO: 1466 - protein, 10 aa, synthetic construct
EILPGSGSTN

SEQ ID NO: 1467 - protein, 15 aa, synthetic construct
ARSGAGGYDSGAFAY

SEQ ID NO: 1468 - protein, 15 aa, synthetic construct
RASESVDTYGNSFMH

SEQ ID NO: 1469 - protein, 8 aa, synthetic construct
YRTSNLES

SEQ ID NO: 1470 - protein, 9 aa, synthetic construct
QQSNEDPWT

SEQ ID NO: 1471 - protein, 8 aa, synthetic construct
GYTFTSYW

SEQ ID NO: 1472 - protein, 8 aa, synthetic construct
ILPGSGST

SEQ ID NO: 1473 - protein, 15 aa, synthetic construct
ARSGAGGYDSGAFAY

SEQ ID NO: 1474 - protein, 10 aa, synthetic construct
ESVDTYGNSF

SEQ ID NO: 1475 - skipped

SEQ ID NO: 1476 - protein, 9 aa, synthetic construct
QQSNEDPWT

SEQ ID NO: 1477 - protein, 5 aa, synthetic construct
SYWIE

SEQ ID NO: 1478 - protein, 17 aa, synthetic construct
EILPGSGSTNYNAKFKG

SEQ ID NO: 1479 - protein, 13 aa, synthetic construct
SGAGGYDSGAFAY

SEQ ID NO: 1480 - protein, 15 aa, synthetic construct
RASESVDTYGNSFMH

SEQ ID NO: 1481 - protein, 7 aa, synthetic construct
RTSNLES

SEQ ID NO: 1482 - protein, 9 aa, synthetic construct
QQSNEDPWT

SEQ ID NO: 1483 - protein, 7 aa, synthetic construct
GYTFTSY

SEQ ID NO: 1484 - protein, 6 aa, synthetic construct
LPGSGS

SEQ ID NO: 1485 - protein, 13 aa, synthetic construct
SGAGGYDSGAFAY

SEQ ID NO: 1486 - protein, 15 aa, synthetic construct
RASESVDTYGNSFMH

SEQ ID NO: 1487 - protein, 7 aa, synthetic construct
RTSNLES

SEQ ID NO: 1488 - protein, 9 aa, synthetic construct
QQSNEDPWT

SEQ ID NO: 1489 - protein, 13 aa, synthetic construct
KASGYTFTSYWIE

SEQ ID NO: 1490 - protein, 10 aa, synthetic construct
EILPGSGSTN

SEQ ID NO: 1491 - protein, 15 aa, synthetic construct
ARSGAGGYDSGAFAY

SEQ ID NO: 1492 - protein, 15 aa, synthetic construct
RASESVDTYGNSFMH

SEQ ID NO: 1493 - protein, 8 aa, synthetic construct
YRTSNLES

SEQ ID NO: 1494 - protein, 9 aa, synthetic construct
QQSNEDPWT

SEQ ID NO: 1495 - protein, 8 aa, synthetic construct
GYTFTSYW

SEQ ID NO: 1496 - protein, 8 aa, synthetic construct
ILPGDEST

SEQ ID NO: 1497 - protein, 15 aa, synthetic construct
ARSGAGGYDSGAFAY

SEQ ID NO: 1498 - protein, 10 aa, synthetic construct
ESVDTYGNSF

SEQ ID NO: 1499 - skipped

SEQ ID NO: 1500 - protein, 9 aa, synthetic construct
QQSNEDPWT

SEQ ID NO: 1501 - protein, 5 aa, synthetic construct
SYWIE

SEQ ID NO: 1502 - protein, 17 aa, synthetic construct
EILPGDESTNYNAKFKG

SEQ ID NO: 1503 - protein, 13 aa, synthetic construct
SGAGGYDSGAFAY

SEQ ID NO: 1504 - protein, 15 aa, synthetic construct
RASESVDTYGNSFMH

SEQ ID NO: 1505 - protein, 7 aa, synthetic construct
RTSNLES

SEQ ID NO: 1506 - protein, 9 aa, synthetic construct
QQSNEDPWT

SEQ ID NO: 1507 - protein, 7 aa, synthetic construct
GYTFTSY

SEQ ID NO: 1508 - protein, 6 aa, synthetic construct
LPGDES

SEQ ID NO: 1509 - protein, 13 aa, synthetic construct
SGAGGYDSGAFAY

SEQ ID NO: 1510 - protein, 15 aa, synthetic construct
RASESVDTYGNSFMH

SEQ ID NO: 1511 - protein, 7 aa, synthetic construct
RTSNLES

SEQ ID NO: 1512 - protein, 9 aa, synthetic construct
QQSNEDPWT

SEQ ID NO: 1513 - protein, 13 aa, synthetic construct
KASGYTFTSYWIE

SEQ ID NO: 1514 - protein, 10 aa, synthetic construct
EILPGDESTN

SEQ ID NO: 1515 - protein, 15 aa, synthetic construct
ARSGAGGYDSGAFAY

SEQ ID NO: 1516 - protein, 15 aa, synthetic construct
RASESVDTYGNSFMH

SEQ ID NO: 1517 - protein, 8 aa, synthetic construct
YRTSNLES

SEQ ID NO: 1518 - protein, 9 aa, synthetic construct
QQSNEDPWT

SEQ ID NO: 1519 - protein, 8 aa, synthetic construct
GYTFTSYW

SEQ ID NO: 1520 - protein, 8 aa, synthetic construct
ILPGDEST

SEQ ID NO: 1521 - protein, 15 aa, synthetic construct
ARSGAGGYDSGAFAY

SEQ ID NO: 1522 - protein, 10 aa, synthetic construct
ESVDTYGNSF

SEQ ID NO: 1523 - skipped

SEQ ID NO: 1524 - protein, 9 aa, synthetic construct
QQSNEDPWT

SEQ ID NO: 1525 - protein, 5 aa, synthetic construct
SYWIE

SEQ ID NO: 1526 - protein, 17 aa, synthetic construct
EILPGDESTNYNAKFKG

SEQ ID NO: 1527 - protein, 13 aa, synthetic construct
SGAGGYDSGAFAY

SEQ ID NO: 1528 - protein, 15 aa, synthetic construct
RASESVDTYGNSFMH

SEQ ID NO: 1529 - protein, 7 aa, synthetic construct
RTSNLES

SEQ ID NO: 1530 - protein, 9 aa, synthetic construct
QQSNEDPWT

SEQ ID NO: 1531 - protein, 7 aa, synthetic construct
GYTFTSY

SEQ ID NO: 1532 - protein, 6 aa, synthetic construct
LPGDES

SEQ ID NO: 1533 - protein, 13 aa, synthetic construct
SGAGGYDSGAFAY

SEQ ID NO: 1534 - protein, 15 aa, synthetic construct
RASESVDTYGNSFMH

SEQ ID NO: 1535 - protein, 7 aa, synthetic construct
RTSNLES

SEQ ID NO: 1536 - protein, 9 aa, synthetic construct
QQSNEDPWT

SEQ ID NO: 1537 - protein, 13 aa, synthetic construct
KASGYTFTSYWIE

SEQ ID NO: 1538 - protein, 10 aa, synthetic construct
EILPGDESTN

SEQ ID NO: 1539 - protein, 15 aa, synthetic construct
ARSGAGGYDSGAFAY

SEQ ID NO: 1540 - protein, 15 aa, synthetic construct
RASESVDTYGNSFMH

SEQ ID NO: 1541 - protein, 8 aa, synthetic construct
YRTSNLES

SEQ ID NO: 1542 - protein, 9 aa, synthetic construct
QQSNEDPWT

SEQ ID NO: 1543 - protein, 8 aa, synthetic construct
GYTFSSYW

SEQ ID NO: 1544 - protein, 8 aa, synthetic construct
ILPGSGST

SEQ ID NO: 1545 - protein, 15 aa, synthetic construct
ARSGAGGYDSGAFAY

SEQ ID NO: 1546 - protein, 10 aa, synthetic construct
ESVDTYGNSF

SEQ ID NO: 1547 - skipped

SEQ ID NO: 1548 - protein, 9 aa, synthetic construct
QQSNEDPWT

SEQ ID NO: 1549 - protein, 5 aa, synthetic construct
SYWIE

SEQ ID NO: 1550 - protein, 17 aa, synthetic construct
EILPGSGSTNYNAKFKG

SEQ ID NO: 1551 - protein, 13 aa, synthetic construct
SGAGGYDSGAFAY

SEQ ID NO: 1552 - protein, 15 aa, synthetic construct
RASESVDTYGNSFMH

SEQ ID NO: 1553 - protein, 7 aa, synthetic construct
RTSNLES

SEQ ID NO: 1554 - protein, 9 aa, synthetic construct
QQSNEDPWT

SEQ ID NO: 1555 - protein, 7 aa, synthetic construct
GYTFSSY

SEQ ID NO: 1556 - protein, 6 aa, synthetic construct
LPGSGS

SEQ ID NO: 1557 - protein, 13 aa, synthetic construct
SGAGGYDSGAFAY

SEQ ID NO: 1558 - protein, 15 aa, synthetic construct
RASESVDTYGNSFMH

SEQ ID NO: 1559 - protein, 7 aa, synthetic construct
RTSNLES

SEQ ID NO: 1560 - protein, 9 aa, synthetic construct
QQSNEDPWT

SEQ ID NO: 1561 - protein, 13 aa, synthetic construct
KASGYTFSSYWIE

SEQ ID NO: 1562 - protein, 10 aa, synthetic construct
EILPGSGSTN

SEQ ID NO: 1563 - protein, 15 aa, synthetic construct
ARSGAGGYDSGAFAY

SEQ ID NO: 1564 - protein, 15 aa, synthetic construct
RASESVDTYGNSFMH

SEQ ID NO: 1565 - protein, 8 aa, synthetic construct
YRTSNLES

SEQ ID NO: 1566 - protein, 9 aa, synthetic construct
QQSNEDPWT

SEQ ID NO: 1567 - protein, 8 aa, synthetic construct
GYTFSSYW

SEQ ID NO: 1568 - protein, 8 aa, synthetic construct
ILPGEGST

SEQ ID NO: 1569 - protein, 15 aa, synthetic construct
ARSGAGGYDSGAFAY

SEQ ID NO: 1570 - protein, 10 aa, synthetic construct
ESVDTYGNSF

SEQ ID NO: 1571 - skipped

SEQ ID NO: 1572 - protein, 9 aa, synthetic construct
QQSNEDPWT

SEQ ID NO: 1573 - protein, 5 aa, synthetic construct
SYWIE

SEQ ID NO: 1574 - protein, 17 aa, synthetic construct
EILPGEGSTNYNAKFKG

SEQ ID NO: 1575 - protein, 13 aa, synthetic construct
SGAGGYDSGAFAY

SEQ ID NO: 1576 - protein, 15 aa, synthetic construct
RASESVDTYGNSFMH

SEQ ID NO: 1577 - protein, 7 aa, synthetic construct
RTSNLES

SEQ ID NO: 1578 - protein, 9 aa, synthetic construct
QQSNEDPWT

SEQ ID NO: 1579 - protein, 7 aa, synthetic construct
GYTFSSY

SEQ ID NO: 1580 - protein, 6 aa, synthetic construct
LPGEGS

SEQ ID NO: 1581 - protein, 13 aa, synthetic construct
SGAGGYDSGAFAY

SEQ ID NO: 1582 - protein, 15 aa, synthetic construct
RASESVDTYGNSFMH

SEQ ID NO: 1583 - protein, 7 aa, synthetic construct
RTSNLES

SEQ ID NO: 1584 - protein, 9 aa, synthetic construct
QQSNEDPWT

SEQ ID NO: 1585 - protein, 13 aa, synthetic construct
KASGYTFSSYWIE

SEQ ID NO: 1586 - protein, 10 aa, synthetic construct
EILPGEGSTN

SEQ ID NO: 1587 - protein, 15 aa, synthetic construct
ARSGAGGYDSGAFAY

SEQ ID NO: 1588 - protein, 15 aa, synthetic construct
RASESVDTYGNSFMH

SEQ ID NO: 1589 - protein, 8 aa, synthetic construct
YRTSNLES

SEQ ID NO: 1590 - protein, 9 aa, synthetic construct
QQSNEDPWT

SEQ ID NO: 1591 - protein, 8 aa, synthetic construct
GYTFSSYW

SEQ ID NO: 1592 - protein, 8 aa, synthetic construct
ILPGDEST

SEQ ID NO: 1593 - protein, 15 aa, synthetic construct
ARSGAGGYDSGAFAY

SEQ ID NO: 1594 - protein, 10 aa, synthetic construct
ESVDTYGNSF

SEQ ID NO: 1595 - skipped

SEQ ID NO: 1596 - protein, 9 aa, synthetic construct
QQSNEDPWT

SEQ ID NO: 1597 - protein, 5 aa, synthetic construct
SYWIE

SEQ ID NO: 1598 - protein, 17 aa, synthetic construct
EILPGDESTNYNAKFKG

SEQ ID NO: 1599 - protein, 13 aa, synthetic construct
SGAGGYDSGAFAY

SEQ ID NO: 1600 - protein, 15 aa, synthetic construct
RASESVDTYGNSFMH

SEQ ID NO: 1601 - protein, 7 aa, synthetic construct
RTSNLES

SEQ ID NO: 1602 - protein, 9 aa, synthetic construct
QQSNEDPWT

SEQ ID NO: 1603 - protein, 7 aa, synthetic construct
GYTFSSY

SEQ ID NO: 1604 - protein, 6 aa, synthetic construct
LPGDES

SEQ ID NO: 1605 - protein, 13 aa, synthetic construct
SGAGGYDSGAFAY

SEQ ID NO: 1606 - protein, 15 aa, synthetic construct
RASESVDTYGNSFMH

SEQ ID NO: 1607 - protein, 7 aa, synthetic construct
RTSNLES

SEQ ID NO: 1608 - protein, 9 aa, synthetic construct
QQSNEDPWT

SEQ ID NO: 1609 - protein, 13 aa, synthetic construct
KASGYTFSSYWIE

SEQ ID NO: 1610 - protein, 10 aa, synthetic construct
EILPGDESTN

SEQ ID NO: 1611 - protein, 15 aa, synthetic construct
ARSGAGGYDSGAFAY

SEQ ID NO: 1612 - protein, 15 aa, synthetic construct
RASESVDTYGNSFMH

SEQ ID NO: 1613 - protein, 8 aa, synthetic construct
YRTSNLES

SEQ ID NO: 1614 - protein, 9 aa, synthetic construct
QQSNEDPWT

SEQ ID NO: 1615 - protein, 8 aa, synthetic construct
GYTFSSYW

SEQ ID NO: 1616 - protein, 8 aa, synthetic construct
ILPGSGST

SEQ ID NO: 1617 - protein, 15 aa, synthetic construct
ARSGAGGYDSGAFAY

SEQ ID NO: 1618 - protein, 10 aa, synthetic construct
ESVDTYGNSF

SEQ ID NO: 1619 - skipped

SEQ ID NO: 1620 - protein, 9 aa, synthetic construct
QQSNEDPWT

SEQ ID NO: 1621 - protein, 5 aa, synthetic construct
SYWIE

SEQ ID NO: 1622 - protein, 17 aa, synthetic construct
EILPGSGSTNYNAKFKG

SEQ ID NO: 1623 - protein, 13 aa, synthetic construct
SGAGGYDSGAFAY

SEQ ID NO: 1624 - protein, 15 aa, synthetic construct
RASESVDTYGNSFMH

SEQ ID NO: 1625 - protein, 7 aa, synthetic construct
RTSNLES

SEQ ID NO: 1626 - protein, 9 aa, synthetic construct
QQSNEDPWT

SEQ ID NO: 1627 - protein, 7 aa, synthetic construct
GYTFSSY

SEQ ID NO: 1628 - protein, 6 aa, synthetic construct
LPGSGS

SEQ ID NO: 1629 - protein, 13 aa, synthetic construct
SGAGGYDSGAFAY

SEQ ID NO: 1630 - protein, 15 aa, synthetic construct
RASESVDTYGNSFMH

SEQ ID NO: 1631 - protein, 7 aa, synthetic construct
RTSNLES

SEQ ID NO: 1632 - protein, 9 aa, synthetic construct
QQSNEDPWT

SEQ ID NO: 1633 - protein, 13 aa, synthetic construct
KASGYTFSSYWIE

SEQ ID NO: 1634 - protein, 10 aa, synthetic construct
EILPGSGSTN

SEQ ID NO: 1635 - protein, 15 aa, synthetic construct
ARSGAGGYDSGAFAY

SEQ ID NO: 1636 - protein, 15 aa, synthetic construct
RASESVDTYGNSFMH

SEQ ID NO: 1637 - protein, 8 aa, synthetic construct
YRTSNLES

SEQ ID NO: 1638 - protein, 9 aa, synthetic construct
QQSNEDPWT

SEQ ID NO: 1639 - protein, 8 aa, synthetic construct
GYTFSSYW

SEQ ID NO: 1640 - protein, 8 aa, synthetic construct
ILPGEGST

SEQ ID NO: 1641 - protein, 15 aa, synthetic construct
ARSGAGGYDSGAFAY

SEQ ID NO: 1642 - protein, 10 aa, synthetic construct
ESVDTYGNSF

SEQ ID NO: 1643 - skipped

SEQ ID NO: 1644 - protein, 9 aa, synthetic construct
QQSNEDPWT

SEQ ID NO: 1645 - protein, 5 aa, synthetic construct
SYWIE

SEQ ID NO: 1646 - protein, 17 aa, synthetic construct
EILPGEGSTNYNAKFKG

SEQ ID NO: 1647 - protein, 13 aa, synthetic construct
SGAGGYDSGAFAY

SEQ ID NO: 1648 - protein, 15 aa, synthetic construct
RASESVDTYGNSFMH

SEQ ID NO: 1649 - protein, 7 aa, synthetic construct
RTSNLES

SEQ ID NO: 1650 - protein, 9 aa, synthetic construct
QQSNEDPWT

SEQ ID NO: 1651 - protein, 7 aa, synthetic construct
GYTFSSY

SEQ ID NO: 1652 - protein, 6 aa, synthetic construct
LPGEGS

SEQ ID NO: 1653 - protein, 13 aa, synthetic construct
SGAGGYDSGAFAY

SEQ ID NO: 1654 - protein, 15 aa, synthetic construct
RASESVDTYGNSFMH

SEQ ID NO: 1655 - protein, 7 aa, synthetic construct
RTSNLES

SEQ ID NO: 1656 - protein, 9 aa, synthetic construct
QQSNEDPWT

SEQ ID NO: 1657 - protein, 13 aa, synthetic construct
KASGYTFSSYWIE

SEQ ID NO: 1658 - protein, 10 aa, synthetic construct
EILPGEGSTN

SEQ ID NO: 1659 - protein, 15 aa, synthetic construct
ARSGAGGYDSGAFAY

SEQ ID NO: 1660 - protein, 15 aa, synthetic construct
RASESVDTYGNSFMH

SEQ ID NO: 1661 - protein, 8 aa, synthetic construct
YRTSNLES

SEQ ID NO: 1662 - protein, 9 aa, synthetic construct
QQSNEDPWT

SEQ ID NO: 1663 - protein, 8 aa, synthetic construct
GYTFSSYW

SEQ ID NO: 1664 - protein, 8 aa, synthetic construct
ILPGDEST

SEQ ID NO: 1665 - protein, 15 aa, synthetic construct
ARSGAGGYDSGAFAY

SEQ ID NO: 1666 - protein, 10 aa, synthetic construct
ESVDTYGNSF

SEQ ID NO: 1667 - skipped

SEQ ID NO: 1668 - protein, 9 aa, synthetic construct
QQSNEDPWT

SEQ ID NO: 1669 - protein, 5 aa, synthetic construct
SYWIE

SEQ ID NO: 1670 - protein, 17 aa, synthetic construct
EILPGDESTNYNAKFKG

SEQ ID NO: 1671 - protein, 13 aa, synthetic construct
SGAGGYDSGAFAY

SEQ ID NO: 1672 - protein, 15 aa, synthetic construct
RASESVDTYGNSFMH

SEQ ID NO: 1673 - protein, 7 aa, synthetic construct
RTSNLES

SEQ ID NO: 1674 - protein, 9 aa, synthetic construct
QQSNEDPWT

SEQ ID NO: 1675 - protein, 7 aa, synthetic construct
GYTFSSY

SEQ ID NO: 1676 - protein, 6 aa, synthetic construct
LPGDES

SEQ ID NO: 1677 - protein, 13 aa, synthetic construct
SGAGGYDSGAFAY

SEQ ID NO: 1678 - protein, 15 aa, synthetic construct
RASESVDTYGNSFMH

SEQ ID NO: 1679 - protein, 7 aa, synthetic construct
RTSNLES

SEQ ID NO: 1680 - protein, 9 aa, synthetic construct
QQSNEDPWT

SEQ ID NO: 1681 - protein, 13 aa, synthetic construct
KASGYTFSSYWIE

SEQ ID NO: 1682 - protein, 10 aa, synthetic construct
EILPGDESTN

SEQ ID NO: 1683 - protein, 15 aa, synthetic construct
ARSGAGGYDSGAFAY

SEQ ID NO: 1684 - protein, 15 aa, synthetic construct
RASESVDTYGNSFMH

SEQ ID NO: 1685 - protein, 8 aa, synthetic construct
YRTSNLES

SEQ ID NO: 1686 - protein, 9 aa, synthetic construct
QQSNEDPWT

SEQ ID NO: 1687 - protein, 127 aa, synthetic construct
QVQLVQSGAEVKKPGASVKVSCKASGYTFTRYVMHWVRQAPGQRLEWIGYINPNNEETKYSQKFKGRVTLTRDTSASTAYMELSSLRSEDTAVYYCARADLEILPYYDHARYAMDYWGQGTLVTVSS

SEQ ID NO: 1688 - protein, 127 aa, synthetic construct
QVQLVQSGAEVKKPGASVKVSCKASGYTFTRYVMHWVRQAPGQRLEWIGYINPNNEETKYNEKFKGRVTLTRDTSASTAYMELSSLRSEDTAVYYCARADLEILPYYDHARYAMDYWGQGTLVTVSS

SEQ ID NO: 1689 - protein, 111 aa, synthetic construct
DIQLTQSPSSLSASVGDRVTITCRASESVDTYGNSFMHWYQQKPGKAPKLLVYRTSNLESGVPSRFSGSGSGTDFTLTISSLQPEDFATYYCQQSNEDPWTFGGGTKVEIK

SEQ ID NO: 1690 - protein, 111 aa, synthetic construct
DIQMTQSPSSLSASVGDRVTITCRASESVDTYGNSFMHWYQQKPGKAPKLLVYRTSNLESGVPSRFSGSGSGTDFTLTISSLQPEDFATYYCQQSNEDPWTFGGGTKVEIK

SEQ ID NO: 1691 - protein, 457 aa, synthetic construct
QVQLVQSGAEVKKPGASVKVSCKASGYTFTRYVMHWVRQAPGQRLEWMGWINPNNEETKYSQKFKGRVTLTRDTSASTAYMELSSLRSEDTAVYYCARADLEILPYYDHARYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1692 - protein, 214 aa, synthetic construct
DIQMTQSPSSVSASVGDRVTITCRASENIYSHLAWYQQKPGKAPKLLVYNAKSLQSGVPSRFSGSGSGTDFTLTISSLQPEDFATYYCQHFWSTPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1693 - protein, 457 aa, synthetic construct
QVQLVQSGAEVKKPGASVKVSCKASGYTFTRYVMHWVRQAPGQRLEWIGYINPNNEETKYSQKFKGRVTLTRDTSASTAYMELSSLRSEDTAVYYCARADLEILPYYDHARYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1694 - protein, 452 aa, synthetic construct
QVQLVQSGAEVKKPGASVKVSCKASGYTFTSYWIEWVRQAPGQGLEWIGEILPGDESTNYNAKFKGRVTFTTDTSTSTAYMELRSLRSDDTAVYYCARSGAGGYDSGAFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1695 - protein, 218 aa, synthetic construct
AIQLTQSPSSLSASVGDRVTITCRASESVDTYGNSFMHWYQQKPGKAPKLLVYRTSNLESGVPSRFSGSGSGTDFTLTISSLQPEDFATYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0171534 A1
(43) Pub. Date: May 29, 2025

(54) ANTI-LILRB1/2 ANTIBODIES AND USES THEREOF

(71) Applicant: D2M Biotherapeutics Limited, Grand Cayman (KY)

(72) Inventors: Dong Zhang, Natick, MA (US); Guangan Hu, Natick, MA (US); Nan Bing, Natick, MA (US); Quanju Zhao; Lai Shi, Natick, MA (US)

(21) Appl. No.: 18/840,622

(22) PCT Filed: Mar. 6, 2023
(86) PCT No.: PCT/US2023/063763

§ 371 Date: Aug. 22, 2024

Related U.S. Application Data
(60) Provisional application No. 63/316,918, filed on Mar. 4, 2022.

(51) Int. Cl. C07K 16/28; A61P 35/00

(52) U.S. Cl. CPC C07K 16/2803; A61P 35/00; C07K 2317/24; C07K 2317/33; C07K 2317/92; C07K 2317/94

(57) ABSTRACT

This disclosure relates to anti-LILRB1 (Leukocyte immunoglobulin-like receptor subfamily B member 1) and/or anti-LILRB2 (Leukocyte immunoglobulin-like receptor subfamily B member 2) antibodies, antigen-binding fragments, and the uses thereof.

### CLAIM OF PRIORITY

This application claims the benefit of U.S. Provisional Application App. No. 63/316,918, filed on Mar. 4, 2022.

### TECHNICAL FIELD

This disclosure relates to antibodies that can bind to Leukocyte immunoglobulin-like receptor subfamily B member 1 and/or Leukocyte immunoglobulin-like receptor subfamily B member 2, and the uses thereof.

### BACKGROUND

Several mechanisms to escape immune destruction are induced throughout tumor progression. For example, nascent transformed cells can lose their antigenicity and/or immunogenicity, and then acquire the expression of inhibitory molecules regulating T cell activation. Secretion of suppressive cytokines can also promote an immunosuppressive microenvironment. Overall, these mechanisms can result in tumor escape via T cell exhaustion and suppressed cell induction.

A breakthrough in cancer therapy to control tumor progression and to re-establish immune function has been developed, e.g., immunotherapies, which are mainly based on antibodies targeting inhibitory molecules regulating T cell activation or their ligands. Successive clinical results have been achieved by administering these antibodies to restore T cell co-stimulatory signals and reactivate anti-tumor T cell responses. Despite the success of these therapies, a considerable proportion of patients remains unresponsive. Thus, there is a need to continue to develop anti-cancer antibodies for use in various antibody-based therapeutics to treat cancers.

### SUMMARY

This disclosure relates to anti-LILRB1/2 antibodies, antigen-binding fragment thereof, and the uses thereof.

In one aspect, the disclosure is related to an antibody or antigen-binding fragment thereof that binds to LILRB1 (Leukocyte immunoglobulin-like receptor subfamily B member 1) and/or LILRB2 (Leukocyte immunoglobulin-like receptor subfamily B member 2), comprising: a heavy chain variable region (VH) comprising complementarity determining regions (CDRs) 1, 2, and 3, in some embodiments, the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and a light chain variable region (VL) comprising CDRs 1, 2, and 3, in some embodiments, the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence, in some embodiments, the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 7, 8, 9, respectively, and the selected VL CDRs 1, 2, 3 amino acid sequences are set forth in SEQ ID NOs: 10, 11, 12, respectively;
(2) the selected VH CDRs 1, 2, 3 amino acid sequences are set forth in SEQ ID NOs: 37, 38, 39, respectively, and the selected VL CDRs 1, 2, 3 amino acid sequences are set forth in SEQ ID NOs: 40, 41, 42, respectively;
(3) the selected VH CDRs 1, 2, 3 amino acid sequences are set forth in SEQ ID NOs: 67, 68, 69, respectively, and the selected VL CDRs 1, 2, 3 amino acid sequences are set forth in SEQ ID NOs: 70, 71, 72, respectively;
(4) the selected VH CDRs 1, 2, 3 amino acid sequences are set forth in SEQ ID NOs: 97, 98, 99, respectively, and the selected VL CDRs 1, 2, 3 amino acid sequences are set forth in SEQ ID NOs: 100, 101, 102, respectively;
(5) the selected VH CDRs 1, 2, 3 amino acid sequences are set forth in SEQ ID NOs: 127, 128, 129, respectively, and the selected VL CDRs 1, 2, 3 amino acid sequences are set forth in SEQ ID NOs: 130, 131, 132, respectively;
(6) the selected VH CDRs 1, 2, 3 amino acid sequences are set forth in SEQ ID NOs: 157, 158, 159, respectively, and the selected VL CDRs 1, 2, 3 amino acid sequences are set forth in SEQ ID NOs: 160, 161, 162, respectively;
(7) the selected VH CDRs 1, 2, 3 amino acid sequences are set forth in SEQ ID NOs: 187, 188, 189, respectively, and the selected VL CDRs 1, 2, 3 amino acid sequences are set forth in SEQ ID NOs: 190, 191, 192, respectively;
(8) the selected VH CDRs 1, 2, 3 amino acid sequences are set forth in SEQ ID NOs: 217, 218, 219, respectively, and the selected VL CDRs 1, 2, 3 amino acid sequences are set forth in SEQ ID NOs: 220, 221, 222, respectively;
(9) the selected VH CDRs 1, 2, 3 amino acid sequences are set forth in SEQ ID NOs: 247, 248, 249, respectively, and the selected VL CDRs 1, 2, 3 amino acid sequences are set forth in SEQ ID NOs: 250, 251, 252, respectively;
(10) the selected VH CDRs 1, 2, 3 amino acid sequences are set forth in SEQ ID NOs: 277, 278, 279, respectively, and the selected VL CDRs 1, 2, 3 amino acid sequences are set forth in SEQ ID NOs: 280, 281, 282, respectively;
(11) the selected VH CDRs 1, 2, 3 amino acid sequences are set forth in SEQ ID NOs: 307, 308, 309, respectively, and the selected VL CDRs 1, 2, 3 amino acid sequences are set forth in SEQ ID NOs: 310, 311, 312, respectively;
(12) the selected VH CDRs 1, 2, 3 amino acid sequences are set forth in SEQ ID NOs: 337, 338, 339, respectively, and the selected VL CDRs 1, 2, 3 amino acid sequences are set forth in SEQ ID NOs: 340, 341, 342, respectively;
(13) the selected VH CDRs 1, 2, 3 amino acid sequences are set forth in SEQ ID NOs: 367, 368, 369, respectively, and the selected VL CDRs 1, 2, 3 amino acid sequences are set forth in SEQ ID NOs: 370, 371, 372, respectively;
(14) the selected VH CDRs 1, 2, 3 amino acid sequences are set forth in SEQ ID NOs: 397, 398, 399, respectively, and the selected VL CDRs 1, 2, 3 amino acid sequences are set forth in SEQ ID NOs: 400, 401, 402, respectively;
(15) the selected VH CDRs 1, 2, 3 amino acid sequences are set forth in SEQ ID NOs: 427, 428, 429, respectively, and the selected VL CDRs 1, 2, 3 amino acid sequences are set forth in SEQ ID NOs: 430, 431, 432, respectively;
(16) the selected VH CDRs 1, 2, 3 amino acid sequences are set forth in SEQ ID NOs: 457, 458, 459, respectively, and the selected VL CDRs 1, 2, 3 amino acid sequences are set forth in SEQ ID NOs: 460, 461, 462, respectively;
(17) the selected VH CDRs 1, 2, 3 amino acid sequences are set forth in SEQ ID NOs: 487, 488, 489, respectively, and the selected VL CDRs 1, 2, 3 amino acid sequences are set forth in SEQ ID NOs: 490, 491, 492, respectively;
(18) the selected VH CDRs 1, 2, 3 amino acid sequences are set forth in SEQ ID NOs: 517, 518, 519, respectively, and the selected VL CDRs 1, 2, 3 amino acid sequences are set forth in SEQ ID NOs: 520, 521, 522, respectively;
(19) the selected VH CDRs 1, 2, 3 amino acid sequences are set forth in SEQ ID NOs: 547, 548, 549, respectively, and the selected VL CDRs 1, 2, 3 amino acid sequences are set forth in SEQ ID NOs: 550, 551, 552, respectively;
(20) the selected VH CDRs 1, 2, 3 amino acid sequences are set forth in SEQ ID NOs: 577, 578, 579, respectively, and the selected VL CDRs 1, 2, 3 amino acid sequences are set forth in SEQ ID NOs: 580, 581, 582, respectively;
(21) the selected VH CDRs 1, 2, 3 amino acid sequences are set forth in SEQ ID NOs: 607, 608, 609, respectively, and the selected VL CDRs 1, 2, 3 amino acid sequences are set forth in SEQ ID NOs: 610, 611, 612, respectively;
(22) the selected VH CDRs 1, 2, 3 amino acid sequences are set forth in SEQ ID NOs: 637, 638, 639, respectively, and the selected VL CDRs 1, 2, 3 amino acid sequences are set forth in SEQ ID NOs: 640, 641, 642, respectively;
(23) the selected VH CDRs 1, 2, 3 amino acid sequences are set forth in SEQ ID NOs: 667, 668, 669, respectively, and the selected VL CDRs 1, 2, 3 amino acid sequences are set forth in SEQ ID NOs: 670, 671, 672, respectively;
(24) the selected VH CDRs 1, 2, 3 amino acid sequences are set forth in SEQ ID NOs: 697, 698, 699, respectively, and the selected VL CDRs 1, 2, 3 amino acid sequences are set forth in SEQ ID NOs: 700, 701, 702, respectively;
(25) the selected VH CDRs 1, 2, 3 amino acid sequences are set forth in SEQ ID NOs: 727, 728, 729, respectively, and the selected VL CDRs 1, 2, 3 amino acid sequences are set forth in SEQ ID NOs: 730, 731, 732, respectively (26) the selected VH CDRs 1, 2, 3 amino acid sequences are set forth in SEQ ID NOs: 1069, 1070, 1071, respectively, and the selected VL CDRs 1, 2, 3 amino acid sequences are set forth in SEQ ID NOs: 1072, 1073, 1074, respectively;
(27) the selected VH CDRs 1, 2, 3 amino acid sequences are set forth in SEQ ID NOs: 1093, 1094, 1095, respectively, and the selected VL CDRs 1, 2, 3 amino acid sequences are set forth in SEQ ID NOs: 1096, 1097, 1098, respectively;
(28) the selected VH CDRs 1, 2, 3 amino acid sequences are set forth in SEQ ID NOs: 1117, 1118, 1119, respectively, and the selected VL CDRs 1, 2, 3 amino acid sequences are set forth in SEQ ID NOs: 1120, 1121, 1122, respectively;
(29) the selected VH CDRs 1, 2, 3 amino acid sequences are set forth in SEQ ID NOs: 1141, 1142, 1143, respectively, and the selected VL CDRs 1, 2, 3 amino acid sequences are set forth in SEQ ID NOs: 1144, 1145, 1146, respectively;
(30) the selected VH CDRs 1, 2, 3 amino acid sequences are set forth in SEQ ID NOs: 1165, 1166, 1167, respectively, and the selected VL CDRs 1, 2, 3 amino acid sequences are set forth in SEQ ID NOs: 1168, 1169, 1170, respectively;
(31) the selected VH CDRs 1, 2, 3 amino acid sequences are set forth in SEQ ID NOs: 1189, 1190, 1191, respectively, and the selected VL CDRs 1, 2, 3 amino acid sequences are set forth in SEQ ID NOs: 1192, 1193, 1194, respectively;
(32) the selected VH CDRs 1, 2, 3 amino acid sequences are set forth in SEQ ID NOs: 1213, 1214, 1215, respectively, and the selected VL CDRs 1, 2, 3 amino acid sequences are set forth in SEQ ID NOs: 1216, 1217, 1218, respectively;
(33) the selected VH CDRs 1, 2, 3 amino acid sequences are set forth in SEQ ID NOs: 1237, 1238, 1239, respectively, and the selected VL CDRs 1, 2, 3 amino acid sequences are set forth in SEQ ID NOs: 1240, 1241, 1242, respectively;
(34) the selected VH CDRs 1, 2, 3 amino acid sequences are set forth in SEQ ID NOs: 1261, 1262, 1263, respectively, and the selected VL CDRs 1, 2, 3 amino acid sequences are set forth in SEQ ID NOs: 1264, 1265, 1266, respectively;
(35) the selected VH CDRs 1, 2, 3 amino acid sequences are set forth in SEQ ID NOs: 1285, 1286, 1287, respectively, and the selected VL CDRs 1, 2, 3 amino acid sequences are set forth in SEQ ID NOs: 1288, 1289, 1290, respectively;
(36) the selected VH CDRs 1, 2, 3 amino acid sequences are set forth in SEQ ID NOs: 1309, 1310, 1311, respectively, and the selected VL CDRs 1, 2, 3 amino acid sequences are set forth in SEQ ID NOs: 1312, 1313, 1314, respectively;
(37) the selected VH CDRs 1, 2, 3 amino acid sequences are set forth in SEQ ID NOs: 1333, 1334, 1335, respectively, and the selected VL CDRs 1, 2, 3 amino acid sequences are set forth in SEQ ID NOs: 1336, 1337, 1338, respectively;
(38) the selected VH CDRs 1, 2, 3 amino acid sequences are set forth in SEQ ID NOs: 1357, 1358, 1359, respectively, and the selected VL CDRs 1, 2, 3 amino acid sequences are set forth in SEQ ID NOs: 1360, 1361, 1362, respectively;
(39) the selected VH CDRs 1, 2, 3 amino acid sequences are set forth in SEQ ID NOs: 1381, 1382, 1383, respectively, and the selected VL CDRs 1, 2, 3 amino acid sequences are set forth in SEQ ID NOs: 1384, 1385, 1386, respectively;
(40) the selected VH CDRs 1, 2, 3 amino acid sequences are set forth in SEQ ID NOs: 1405, 1406, 1407, respectively, and the selected VL CDRs 1, 2, 3 amino acid sequences are set forth in SEQ ID NOs: 1408, 1409, 1410, respectively;
(41) the selected VH CDRs 1, 2, 3 amino acid sequences are set forth in SEQ ID NOs: 1429, 1430, 1431, respectively, and the selected VL CDRs 1, 2, 3 amino acid sequences are set forth in SEQ ID NOs: 1432, 1433, 1434, respectively;
(42) the selected VH CDRs 1, 2, 3 amino acid sequences are set forth in SEQ ID NOs: 1453, 1454, 1455, respectively, and the selected VL CDRs 1, 2, 3 amino acid sequences are set forth in SEQ ID NOs: 1456, 1457, 1458, respectively;
(43) the selected VH CDRs 1, 2, 3 amino acid sequences are set forth in SEQ ID NOs: 1477, 1478, 1479, respectively, and the selected VL CDRs 1, 2, 3 amino acid sequences are set forth in SEQ ID NOs: 1480, 1481, 1482, respectively;
(44) the selected VH CDRs 1, 2, 3 amino acid sequences are set forth in SEQ ID NOs: 1501, 1502, 1503, respectively, and the selected VL CDRs 1, 2, 3 amino acid sequences are set forth in SEQ ID NOs: 1504, 1505, 1506, respectively;
(45) the selected VH CDRs 1, 2, 3 amino acid sequences are set forth in SEQ ID NOs: 1525, 1526, 1527, respectively, and the selected VL CDRs 1, 2, 3 amino acid sequences are set forth in SEQ ID NOs: 1528, 1529, 1530, respectively;
(46) the selected VH CDRs 1, 2, 3 amino acid sequences are set forth in SEQ ID NOs: 1549, 1550, 1551, respectively, and the selected VL CDRs 1, 2, 3 amino acid sequences are set forth in SEQ ID NOs: 1552, 1553, 1554, respectively;
(47) the selected VH CDRs 1, 2, 3 amino acid sequences are set forth in SEQ ID NOs: 1573, 1574, 1575, respectively, and the selected VL CDRs 1, 2, 3 amino acid sequences are set forth in SEQ ID NOs: 1576, 1577, 1578, respectively;
(48) the selected VH CDRs 1, 2, 3 amino acid sequences are set forth in SEQ ID NOs: 1597, 1598, 1599, respectively, and the selected VL CDRs 1, 2, 3 amino acid sequences are set forth in SEQ ID NOs: 1600, 1601, 1602, respectively;
(49) the selected VH CDRs 1, 2, 3 amino acid sequences are set forth in SEQ ID NOs: 1621, 1622, 1623, respectively, and the selected VL CDRs 1, 2, 3 amino acid sequences are set forth in SEQ ID NOs: 1624, 1625, 1626, respectively;
(50) the selected VH CDRs 1, 2, 3 amino acid sequences are set forth in SEQ ID NOs: 1645, 1646, 1647, respectively, and the selected VL CDRs 1, 2, 3 amino acid sequences are set forth in SEQ ID NOs: 1648, 1649, 1650, respectively; and
(51) the selected VH CDRs 1, 2, 3 amino acid sequences are set forth in SEQ ID NOs: 1669, 1670, 1671, respectively, and the selected VL CDRs 1, 2, 3 amino acid sequences are set forth in SEQ ID NOs: 1672, 1673, 1674, respectively.

In some embodiments, the VH comprises CDRs 1, 2, 3 with the amino acid sequences set forth in SEQ ID NOs: 7, 8, and 9 respectively, and the VL comprises CDRs 1, 2, 3 with the amino acid sequences set forth in SEQ ID NOs: 10, 11, and 12, respectively. In some embodiments, the VH comprises CDRs 1, 2, 3 with the amino acid sequences set forth in SEQ ID NOs: 37, 38, and 39 respectively, and the VL comprises CDRs 1, 2, 3 with the amino acid sequences set forth in SEQ ID NOs: 40, 41, and 42, respectively. In some embodiments, the VH comprises CDRs 1, 2, 3 with the amino acid sequences set forth in SEQ ID NOs: 67, 68, and 69 respectively, and the VL comprises CDRs 1, 2, 3 with the amino acid sequences set forth in SEQ ID NOs: 70, 71, and 72, respectively. In some embodiments, the VH comprises CDRs 1, 2, 3 with the amino acid sequences set forth in SEQ ID NOs: 97, 98, and 99 respectively, and the VL comprises CDRs 1, 2, 3 with the amino acid sequences set forth in SEQ ID NOs: 100, 101, and 102, respectively. In some embodiments, the VH comprises CDRs 1, 2, 3 with the amino acid sequences set forth in SEQ ID NOs: 127, 128, and 129 respectively, and the VL comprises CDRs 1, 2, 3 with the amino acid sequences set forth in SEQ ID NOs: 130, 131, and 132, respectively.

In some embodiments, the VH comprises CDRs 1, 2, 3 with the amino acid sequences set forth in SEQ ID NOs: 157, 158, and 159 respectively, and the VL comprises CDRs 1, 2, 3 with the amino acid sequences set forth in SEQ ID NOs: 160, 161, and 162, respectively. In some embodiments, the VH comprises CDRs 1, 2, 3 with the amino acid sequences set forth in SEQ ID NOs: 187, 188, and 189 respectively, and the VL comprises CDRs 1, 2, 3 with the amino acid sequences set forth in SEQ ID NOs: 190, 191, and 192, respectively. In some embodiments, the VH comprises CDRs 1, 2, 3 with the amino acid sequences set forth in SEQ ID NOs: 217, 218, and 219 respectively, and the VL comprises CDRs 1, 2, 3 with the amino acid sequences set forth in SEQ ID NOs: 220, 221, and 222, respectively. In some embodiments, the VH comprises CDRs 1, 2, 3 with the amino acid sequences set forth in SEQ ID NOs: 247, 248, and 249 respectively, and the VL comprises CDRs 1, 2, 3 with the amino acid sequences set forth in SEQ ID NOs: 250, 251, and 252, respectively. In some embodiments, the VH comprises CDRs 1, 2, 3 with the amino acid sequences set forth in SEQ ID NOs: 277, 278, and 279 respectively, and the VL comprises CDRs 1, 2, 3 with the amino acid sequences set forth in SEQ ID NOs: 280, 281, and 282, respectively. In some embodiments, the VH comprises CDRs 1, 2, 3 with the amino acid sequences set forth in SEQ ID NOs: 307, 308, and 309 respectively, and the VL comprises CDRs 1, 2, 3 with the amino acid sequences set forth in SEQ ID NOs: 310, 311, and 312, respectively. In some embodiments, the VH comprises CDRs 1, 2, 3 with the amino acid sequences set forth in SEQ ID NOs: 337, 338, and 339 respectively, and the VL comprises CDRs 1, 2, 3 with the amino acid sequences set forth in SEQ ID NOs: 340, 341, and 342, respectively. In some embodiments, the VH comprises CDRs 1, 2, 3 with the amino acid sequences set forth in SEQ ID NOs: 367, 368, and 369 respectively, and the VL comprises CDRs 1, 2, 3 with the amino acid sequences set forth in SEQ ID NOs: 370, 371, and 372, respectively. In some embodiments, the VH comprises CDRs 1, 2, 3 with the amino acid sequences set forth in SEQ ID NOs: 397, 398, and 399 respectively, and the VL comprises CDRs 1, 2, 3 with the amino acid sequences set forth in SEQ ID NOs: 400, 401, and 402, respectively. In some embodiments, the VH comprises CDRs 1, 2, 3 with the amino acid sequences set forth in SEQ ID NOs: 427, 428, and 429 respectively, and the VL comprises CDRs 1, 2, 3 with the amino acid sequences set forth in SEQ ID NOs: 430, 431, and 432, respectively. In some embodiments, the VH comprises CDRs 1, 2, 3 with the amino acid sequences set forth in SEQ ID NOs: 457, 458, and 459 respectively, and the VL comprises CDRs 1, 2, 3 with the amino acid sequences set forth in SEQ ID NOs: 460, 461, and 462, respectively. In some embodiments, the VH comprises CDRs 1, 2, 3 with the amino acid sequences set forth in SEQ ID NOs: 487, 488, and 489 respectively, and the VL comprises CDRs 1, 2, 3 with the amino acid sequences set forth in SEQ ID NOs: 490, 491, and 492, respectively. In some embodiments, the VH comprises CDRs 1, 2, 3 with the amino acid sequences set forth in SEQ ID NOs: 517, 518, and 519 respectively, and the VL comprises CDRs 1, 2, 3 with the amino acid sequences set forth in SEQ ID NOs: 520, 521, and 522, respectively. In some embodiments, the VH comprises CDRs 1, 2, 3 with the amino acid sequences set forth in SEQ ID NOs: 547, 548, and 549 respectively, and the VL comprises CDRS 1, 2, 3 with the amino acid sequences set forth in SEQ ID NOs: 550, 551, and 552, respectively. In some embodiments, the VH comprises CDRs 1, 2, 3 with the amino acid sequences set forth in SEQ ID NOs: 577, 578, and 579 respectively, and the VL comprises CDRs 1, 2, 3 with the amino acid sequences set forth in SEQ ID NOs: 580, 581, and 582, respectively. In some embodiments, the VH comprises CDRs 1, 2, 3 with the amino acid sequences set forth in SEQ ID NOs: 607, 608, and 609 respectively, and the VL comprises CDRs 1, 2, 3 with the amino acid sequences set forth in SEQ ID NOs: 610, 611, and 612, respectively. In some embodiments, the VH comprises CDRs 1, 2, 3 with the amino acid sequences set forth in SEQ ID NOs: 637, 638, and 639 respectively, and the VL comprises CDRs 1, 2, 3 with the amino acid sequences set forth in SEQ ID NOs: 640, 641, and 642, respectively. In some embodiments, the VH comprises CDRs 1, 2, 3 with the amino acid sequences set forth in SEQ ID NOs: 667, 668, and 669 respectively, and the VL comprises CDRs 1, 2, 3 with the amino acid sequences set forth in SEQ ID NOs: 670, 671, and 672, respectively. In some embodiments, the VH comprises CDRs 1, 2, 3 with the amino acid sequences set forth in SEQ ID NOs: 697, 698, and 699 respectively, and the VL comprises CDRs 1, 2, 3 with the amino acid sequences set forth in SEQ ID NOs: 700, 701, and 702, respectively. In some embodiments, the VH comprises CDRs 1, 2, 3 with the amino acid sequences set forth in SEQ ID NOs: 727, 728, and 729 respectively, and the VL comprises CDRs 1, 2, 3 with the amino acid sequences set forth in SEQ ID NOs: 730, 731, and 732, respectively. In some embodiments the VH comprises CDRs 1, 2, 3 with the amino acid sequences set forth in SEQ ID NOs: 1069, 1070, and 1071, respectively, and the VL comprises CDRs 1, 2, 3 with the amino acid sequences set forth in SEQ ID NOs: 1072, 1073, and 1074, respectively. In some embodiments the VH comprises CDRs 1, 2, 3 with the amino acid sequences set forth in SEQ ID NOs: 1093, 1094, and 1095, respectively, and the VL comprises CDRs 1, 2, 3 with the amino acid sequences set forth in SEQ ID NOs: 1096, 1097, and 1098, respectively. In some embodiments the VH comprises CDRs 1, 2, 3 with the amino acid sequences set forth in SEQ ID NOs: 1117, 1118, and 1119, respectively, and the VL comprises CDRs 1, 2, 3 with the amino acid sequences set forth in SEQ ID NOs: 1120, 1121, and 1122, respectively. In some embodiments the VH comprises CDRs 1, 2, 3 with the amino acid sequences set forth in SEQ ID NOs: 1141, 1142, and 1143, respectively, and the VL comprises CDRs 1, 2, 3 with the amino acid sequences set forth in SEQ ID NOs: 1144, 1145, and 1146, respectively. In some embodiments the VH comprises CDRs 1, 2, 3 with the amino acid sequences set forth in SEQ ID NOs: 1165, 1166, and 1167, respectively, and the VL comprises CDRs 1, 2, 3 with the amino acid sequences set forth in SEQ ID NOs: 1168, 1169, and 1170, respectively. In some embodiments the VH comprises CDRs 1, 2, 3 with the amino acid sequences set forth in SEQ ID NOs: 1189, 1190, and 1191, respectively, and the VL comprises CDRs 1, 2, 3 with the amino acid sequences set forth in SEQ ID NOs: 1192, 1193, and 1194, respectively. In some embodiments the VH comprises CDRs 1, 2, 3 with the amino acid sequences set forth in SEQ ID NOs: 1213, 1214, and 1215, respectively, and the VL comprises CDRs 1, 2, 3 with the amino acid sequences set forth in SEQ ID NOs: 1216, 1217, and 1218, respectively. In some embodiments the VH comprises CDRs 1, 2, 3 with the amino acid sequences set forth in SEQ ID NOs: 1237, 1238, and 1239, respectively, and the VL comprises CDRs 1, 2, 3 with the amino acid sequences set forth in SEQ ID NOs: 1240, 1241, and 1242, respectively. In some embodiments the VH comprises CDRs 1, 2, 3 with the amino acid sequences set forth in SEQ ID NOs: 1261, 1262, and 1263, respectively, and the VL comprises CDRs 1, 2, 3 with the amino acid sequences set forth in SEQ ID NOs: 1264, 1265, and 1266, respectively. In some embodiments the VH comprises CDRs 1, 2, 3 with the amino acid sequences set forth in SEQ ID NOs: 1285, 1286, and 1287, respectively, and the VL comprises CDRs 1, 2, 3 with the amino acid sequences set forth in SEQ ID NOs: 1288, 1289, and 1290, respectively. In some embodiments the VH comprises CDRs 1, 2, 3 with the amino acid sequences set forth in SEQ ID NOs: 1309, 1310, and 1311, respectively, and the VL comprises CDRs 1, 2, 3 with the amino acid sequences set forth in SEQ ID NOs: 1312, 1313, and 1314, respectively. In some embodiments the VH comprises CDRs 1, 2, 3 with the amino acid sequences set forth in SEQ ID NOs: 1333, 1334, and 1335, respectively, and the VL comprises CDRs 1, 2, 3 with the amino acid sequences set forth in SEQ ID NOs: 1336, 1337, and 1338, respectively. In some embodiments the VH comprises CDRs 1, 2, 3 with the amino acid sequences set forth in SEQ ID NOs: 1357, 1358, and 1359, respectively, and the VL comprises CDRs 1, 2, 3 with the amino acid sequences set forth in SEQ ID NOs: 1360, 1361, and 1362, respectively. In some embodiments the VH comprises CDRs 1, 2, 3 with the amino acid sequences set forth in SEQ ID NOs: 1381, 1382, and 1383, respectively, and the VL comprises CDRs 1, 2, 3 with the amino acid sequences set forth in SEQ ID NOs: 1384, 1385, and 1386, respectively. In some embodiments the VH comprises CDRs 1, 2, 3 with the amino acid sequences set forth in SEQ ID NOs: 1405, 1406, and 1407, respectively, and the VL comprises CDRs 1, 2, 3 with the amino acid sequences set forth in SEQ ID NOs: 1408, 1409, and 1410, respectively. In some embodiments the VH comprises CDRs 1, 2, 3 with the amino acid sequences set forth in SEQ ID NOs: 1429, 1430, and 1431, respectively, and the VL comprises CDRs 1, 2, 3 with the amino acid sequences set forth in SEQ ID NOs: 1432, 1433, and 1434, respectively. In some embodiments the VH comprises CDRs 1, 2, 3 with the amino acid sequences set forth in SEQ ID NOs: 1453, 1454, and 1455, respectively, and the VL comprises CDRs 1, 2, 3 with the amino acid sequences set forth in SEQ ID NOs: 1456, 1457, and 1458, respectively. In some embodiments the VH comprises CDRs 1, 2, 3 with the amino acid sequences set forth in SEQ ID NOs: 1477, 1478, and 1479, respectively, and the VL comprises CDRs 1, 2, 3 with the amino acid sequences set forth in SEQ ID NOs: 1480, 1481, and 1482, respectively. In some embodiments the VH comprises CDRs 1, 2, 3 with the amino acid sequences set forth in SEQ ID NOs: 1501, 1502, and 1503, respectively, and the VL comprises CDRs 1, 2, 3 with the amino acid sequences set forth in SEQ ID NOs: 1504, 1505, and 1506, respectively. In some embodiments the VH comprises CDRs 1, 2, 3 with the amino acid sequences set forth in SEQ ID NOs: 1525, 1526, and 1527, respectively, and the VL comprises CDRs 1, 2, 3 with the amino acid sequences set forth in SEQ ID NOs: 1528, 1529, and 1530, respectively. In some embodiments the VH comprises CDRs 1, 2, 3 with the amino acid sequences set forth in SEQ ID NOs: 1549, 1550, and 1551, respectively, and the VL comprises CDRs 1, 2, 3 with the amino acid sequences set forth in SEQ ID NOs: 1552, 1553, and 1554, respectively. In some embodiments the VH comprises CDRs 1, 2, 3 with the amino acid sequences set forth in SEQ ID NOs: 1573, 1574, and 1575, respectively, and the VL comprises CDRs 1, 2, 3 with the amino acid sequences set forth in SEQ ID NOs: 1576, 1577, and 1578, respectively. In some embodiments the VH comprises CDRs 1, 2, 3 with the amino acid sequences set forth in SEQ ID NOs: 1597, 1598, and 1599, respectively, and the VL comprises CDRs 1, 2, 3 with the amino acid sequences set forth in SEQ ID NOs: 1600, 1601, and 1602, respectively. In some embodiments the VH comprises CDRs 1, 2, 3 with the amino acid sequences set forth in SEQ ID NOs: 1621, 1622, and 1623, respectively, and the VL comprises CDRs 1, 2, 3 with the amino acid sequences set forth in SEQ ID NOs: 1624, 1625, and 1626, respectively. In some embodiments the VH comprises CDRs 1, 2, 3 with the amino acid sequences set forth in SEQ ID NOs: 1645, 1646, and 1647, respectively, and the VL comprises CDRs 1, 2, 3 with the amino acid sequences set forth in SEQ ID NOs: 1648, 1649, and 1650, respectively. In some embodiments the VH comprises CDRs 1, 2, 3 with the amino acid sequences set forth in SEQ ID NOs: 1669, 1670, and 1671, respectively, and the VL comprises CDRs 1, 2, 3 with the amino acid sequences set forth in SEQ ID NOs: 1672, 1673, and 1674, respectively.

In some embodiments, the antibody or antigen-binding fragment thereof specifically binds to human LILRB1 and/or human LILRB2. In some embodiments, the antibody or antigen-binding fragment thereof specifically binds to monkey LILRB1 and/or monkey LILRB2. In some embodiments, the antibody or antigen-binding fragment thereof can block the interaction between LILRB1 and MHC Class I molecules (e.g., HLA-G), or the interaction between LILRB2 and MHC Class I molecules (e.g., HLA-G). In some embodiments, the antibody or antigen-binding fragment thereof is a humanized antibody or antigen-binding fragment thereof. In some embodiments, the antibody or antigen-binding fragment thereof is a single-chain variable fragment (scFV) or a multi-specific antibody (e.g., a bispecific antibody). In some embodiments, the antibody or antigen-binding fragment thereof specifically binds to LILRB3 (e.g., human LILRB3).

In one aspect, the disclosure is related to a nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 7, 8, and 9, respectively, and in some embodiments, the VH, when paired with a light chain variable region (VL) comprising the amino acid sequence set forth in SEQ ID NO: 752 binds to LILRB1 and/or LILRB2;
(2) an immunoglobulin light chain or a fragment thereof comprising a VL comprising CDRs 1, 2, and 3 comprising the amino acid sequences set forth in SEQ ID NOs: 10, 11, and 12, respectively, and in some embodiments, the VL, when paired with a VH comprising the amino acid sequence set forth in SEQ ID NO: 751 binds to LILRB1 and/or LILRB2;
(3) an immunoglobulin heavy chain or a fragment thereof comprising a VH comprising CDRs 1, 2, and 3 comprising the amino acid sequences set forth in SEQ ID NOs: 37, 38, and 39, respectively, and in some embodiments, the VH, when paired with a VL comprising the amino acid sequence set forth in SEQ ID NO: 754 binds to LILRB1 and/or LILRB2;
(4) an immunoglobulin light chain or a fragment thereof comprising a VL comprising CDRs 1, 2, and 3 comprising the amino acid sequences set forth in SEQ ID NOs: 40, 41, and 42, respectively, and in some embodiments, the VL, when paired with a VH comprising the amino acid sequence set forth in SEQ ID NO: 753 binds to LILRB1 and/or LILRB2;
(5) an immunoglobulin heavy chain or a fragment thereof comprising a VH comprising CDRs 1, 2, and 3 comprising the amino acid sequences set forth in SEQ ID NOs: 67, 68, and 69, respectively, and in some embodiments, the VH, when paired with a VL comprising the amino acid sequence set forth in SEQ ID NO: 756 binds to LILRB1 and/or LILRB2;
(6) an immunoglobulin light chain or a fragment thereof comprising a VL comprising CDRs 1, 2, and 3 comprising the amino acid sequences set forth in SEQ ID NOs: 70, 71, and 72, respectively, and in some embodiments, the VL, when paired with a VH comprising the amino acid sequence set forth in SEQ ID NO: 755 binds to LILRB1 and/or LILRB2;
(7) an immunoglobulin heavy chain or a fragment thereof comprising a VH comprising CDRs 1, 2, and 3 comprising the amino acid sequences set forth in SEQ ID NOs: 97, 98, and 99, respectively, and in some embodiments, the VH, when paired with a VL comprising the amino acid sequence set forth in SEQ ID NO: 758 binds to LILRB1 and/or LILRB2;
(8) an immunoglobulin light chain or a fragment thereof comprising a VL comprising CDRs 1, 2, and 3 comprising the amino acid sequences set forth in SEQ ID NOs: 100, 101, and 102, respectively, and in some embodiments, the VL, when paired with a VH comprising the amino acid sequence set forth in SEQ ID NO: 757 binds to LILRB1 and/or LILRB2;
(9) an immunoglobulin heavy chain or a fragment thereof comprising a VH comprising CDRs 1, 2, and 3 comprising the amino acid sequences set forth in SEQ ID NOs: 127, 128, and 129, respectively, and in some embodiments, the VH, when paired with a VL comprising the amino acid sequence set forth in SEQ ID NO: 760 binds to LILRB1 and/or LILRB2;
(10) an immunoglobulin light chain or a fragment thereof comprising a VL comprising CDRs 1, 2, and 3 comprising the amino acid sequences set forth in SEQ ID NOs: 130, 131, and 132, respectively, and in some embodiments, the VL, when paired with a VH comprising the amino acid sequence set forth in SEQ ID NO: 759 binds to LILRB1 and/or LILRB2;
(11) an immunoglobulin heavy chain or a fragment thereof comprising a VH comprising CDRs 1, 2, and 3 comprising the amino acid sequences set forth in SEQ ID NOs: 157, 158, and 159, respectively, and in some embodiments, the VH, when paired with a VL comprising the amino acid sequence set forth in SEQ ID NO: 762 binds to LILRB1 and/or LILRB2;
(12) an immunoglobulin light chain or a fragment thereof comprising a VL comprising CDRs 1, 2, and 3 comprising the amino acid sequences set forth in SEQ ID NOs: 160, 161, and 162, respectively, and in some embodiments, the VL, when paired with a VH comprising the amino acid sequence set forth in SEQ ID NO: 761 binds to LILRB1 and/or LILRB2;
(13) an immunoglobulin heavy chain or a fragment thereof comprising a VH comprising CDRs 1, 2, and 3 comprising the amino acid sequences set forth in SEQ ID NOs: 187, 188, and 189, respectively, and in some embodiments, the VH, when paired with a VL comprising the amino acid sequence set forth in SEQ ID NO: 764 binds to LILRB1 and/or LILRB2;
(14) an immunoglobulin light chain or a fragment thereof comprising a VL comprising CDRs 1, 2, and 3 comprising the amino acid sequences set forth in SEQ ID NOs: 190, 191, and 192, respectively, and in some embodiments, the VL, when paired with a VH comprising the amino acid sequence set forth in SEQ ID NO: 763 binds to LILRB1 and/or LILRB2;
(15) an immunoglobulin heavy chain or a fragment thereof comprising a VH comprising CDRs 1, 2, and 3 comprising the amino acid sequences set forth in SEQ ID NOs: 217, 218, and 219, respectively, and in some embodiments, the VH, when paired with a VL comprising the amino acid sequence set forth in SEQ ID NO: 766 binds to LILRB1 and/or LILRB2;
(16) an immunoglobulin light chain or a fragment thereof comprising a VL comprising CDRs 1, 2, and 3 comprising the amino acid sequences set forth in SEQ ID NOs: 220, 221, and 222, respectively, and in some embodiments, the VL, when paired with a VH comprising the amino acid sequence set forth in SEQ ID NO: 765 binds to LILRB1 and/or LILRB2;
(17) an immunoglobulin heavy chain or a fragment thereof comprising a VH comprising CDRs 1, 2, and 3 comprising the amino acid sequences set forth in SEQ ID NOs: 247, 248, and 249, respectively, and in some embodiments, the VH, when paired with a VL comprising the amino acid sequence set forth in SEQ ID NO: 768 binds to LILRB1 and/or LILRB2;
(18) an immunoglobulin light chain or a fragment thereof comprising a VL comprising CDRs 1, 2, and 3 comprising the amino acid sequences set forth in SEQ ID NOs: 250, 251, and 252, respectively, and in some embodiments, the VL, when paired with a VH comprising the amino acid sequence set forth in SEQ ID NO: 767 binds to LILRB1 and/or LILRB2;
(19) an immunoglobulin heavy chain or a fragment thereof comprising a VH comprising CDRs 1, 2, and 3 comprising the amino acid sequences set forth in SEQ ID NOs: 277, 278, and 279, respectively, and in some embodiments, the VH, when paired with a VL comprising the amino acid sequence set forth in SEQ ID NO: 770 binds to LILRB1 and/or LILRB2;
(20) an immunoglobulin light chain or a fragment thereof comprising a VL comprising CDRs 1, 2, and 3 comprising the amino acid sequences set forth in SEQ ID NOs: 280, 281, and 282, respectively, and in some embodiments, the VL, when paired with a VH comprising the amino acid sequence set forth in SEQ ID NO: 769 binds to LILRB1 and/or LILRB2;
(21) an immunoglobulin heavy chain or a fragment thereof comprising a VH comprising CDRs 1, 2, and 3 comprising the amino acid sequences set forth in SEQ ID NOs: 307, 308, and 309, respectively, and in some embodiments, the VH, when paired with a VL comprising the amino acid sequence set forth in SEQ ID NO: 772 binds to LILRB1 and/or LILRB2;
(22) an immunoglobulin light chain or a fragment thereof comprising a VL comprising CDRs 1, 2, and 3 comprising the amino acid sequences set forth in SEQ ID NOs: 310, 311, and 312, respectively, and in some embodiments, the VL, when paired with a VH comprising the amino acid sequence set forth in SEQ ID NO: 771 binds to LILRB1 and/or LILRB2;
(23) an immunoglobulin heavy chain or a fragment thereof comprising a VH comprising CDRs 1, 2, and 3 comprising the amino acid sequences set forth in SEQ ID NOs: 337, 338, and 339, respectively, and in some embodiments, the VH, when paired with a VL comprising the amino acid sequence set forth in SEQ ID NO: 774 binds to LILRB1 and/or LILRB2;
(24) an immunoglobulin light chain or a fragment thereof comprising a VL comprising CDRs 1, 2, and 3 comprising the amino acid sequences set forth in SEQ ID NOs: 340, 341, and 342, respectively, and in some embodiments, the VL, when paired with a VH comprising the amino acid sequence set forth in SEQ ID NO: 773 binds to LILRB1 and/or LILRB2;
(25) an immunoglobulin heavy chain or a fragment thereof comprising a VH comprising CDRs 1, 2, and 3 comprising the amino acid sequences set forth in SEQ ID NOs: 367, 368, and 369, respectively, and in some embodiments, the VH, when paired with a VL comprising the amino acid sequence set forth in SEQ ID NO: 776 binds to LILRB1 and/or LILRB2;
(26) an immunoglobulin light chain or a fragment thereof comprising a VL comprising CDRs 1, 2, and 3 comprising the amino acid sequences set forth in SEQ ID NOs: 370, 371, and 372, respectively, and in some embodiments, the VL, when paired with a VH comprising the amino acid sequence set forth in SEQ ID NO: 775 binds to LILRB1 and/or LILRB2;
(27) an immunoglobulin heavy chain or a fragment thereof comprising a VH comprising CDRs 1, 2, and 3 comprising the amino acid sequences set forth in SEQ ID NOs: 397, 398, and 399, respectively, and in some embodiments, the VH, when paired with a VL comprising the amino acid sequence set forth in SEQ ID NO: 778 binds to LILRB1 and/or LILRB2;
(28) an immunoglobulin light chain or a fragment thereof comprising a VL comprising CDRs 1, 2, and 3 comprising the amino acid sequences set forth in SEQ ID NOs: 400, 401, and 402, respectively, and in some embodiments, the VL, when paired with a VH comprising the amino acid sequence set forth in SEQ ID NO: 777 binds to LILRB1 and/or LILRB2;
(29) an immunoglobulin heavy chain or a fragment thereof comprising a VH comprising CDRs 1, 2, and 3 comprising the amino acid sequences set forth in SEQ ID NOs: 427, 428, and 429, respectively, and in some embodiments, the VH, when paired with a VL comprising the amino acid sequence set forth in SEQ ID NO: 780 binds to LILRB1 and/or LILRB2;
(30) an immunoglobulin light chain or a fragment thereof comprising a VL comprising CDRs 1, 2, and 3 comprising the amino acid sequences set forth in SEQ ID NOs: 430, 431, and 432, respectively, and in some embodiments, the VL, when paired with a VH comprising the amino acid sequence set forth in SEQ ID NO: 779 binds to LILRB1 and/or LILRB2;
(31) an immunoglobulin heavy chain or a fragment thereof comprising a VH comprising CDRs 1, 2, and 3 comprising the amino acid sequences set forth in SEQ ID NOs: 457, 458, and 459, respectively, and in some embodiments, the VH, when paired with a VL comprising the amino acid sequence set forth in SEQ ID NO: 782 binds to LILRB1 and/or LILRB2;
(32) an immunoglobulin light chain or a fragment thereof comprising a VL comprising CDRs 1, 2, and 3 comprising the amino acid sequences set forth in SEQ ID NOs: 460, 461, and 462, respectively, and in some embodiments, the VL, when paired with a VH comprising the amino acid sequence set forth in SEQ ID NO: 781 binds to LILRB1 and/or LILRB2;
(33) an immunoglobulin heavy chain or a fragment thereof comprising a VH comprising CDRs 1, 2, and 3 comprising the amino acid sequences set forth in SEQ ID NOs: 487, 488, and 489, respectively, and in some embodiments, the VH, when paired with a VL comprising the amino acid sequence set forth in SEQ ID NO: 784 binds to LILRB1 and/or LILRB2;
(34) an immunoglobulin light chain or a fragment thereof comprising a VL comprising CDRs 1, 2, and 3 comprising the amino acid sequences set forth in SEQ ID NOs: 490, 491, and 492, respectively, and in some embodiments, the VL, when paired with a VH comprising the amino acid sequence set forth in SEQ ID NO: 783 binds to LILRB1 and/or LILRB2;
(35) an immunoglobulin heavy chain or a fragment thereof comprising a VH comprising CDRs 1, 2, and 3 comprising the amino acid sequences set forth in SEQ ID NOs: 517, 518, and 519, respectively, and in some embodiments, the VH, when paired with a VL comprising the amino acid sequence set forth in SEQ ID NO: 786 binds to LILRB1 and/or LILRB2;
(36) an immunoglobulin light chain or a fragment thereof comprising a VL comprising CDRs 1, 2, and 3 comprising the amino acid sequences set forth in SEQ ID NOs: 520, 521, and 522, respectively, and in some embodiments, the VL, when paired with a VH comprising the amino acid sequence set forth in SEQ ID NO: 785 binds to LILRB1 and/or LILRB2;
(37) an immunoglobulin heavy chain or a fragment thereof comprising a VH comprising CDRs 1, 2, and 3 comprising the amino acid sequences set forth in SEQ ID NOs: 547, 548, and 549, respectively, and in some embodiments, the VH, when paired with a VL comprising the amino acid sequence set forth in SEQ ID NO: 788 binds to LILRB1 and/or LILRB2;
(38) an immunoglobulin light chain or a fragment thereof comprising a VL comprising CDRs 1, 2, and 3 comprising the amino acid sequences set forth in SEQ ID NOs: 550, 551, and 552, respectively, and in some embodiments, the VL, when paired with a VH comprising the amino acid sequence set forth in SEQ ID NO: 787 binds to LILRB1 and/or LILRB2;
(39) an immunoglobulin heavy chain or a fragment thereof comprising a VH comprising CDRs 1, 2, and 3 comprising the amino acid sequences set forth in SEQ ID NOs: 577, 578, and 579, respectively, and in some embodiments, the VH, when paired with a VL comprising the amino acid sequence set forth in SEQ ID NO: 790 binds to LILRB1 and/or LILRB2;
(40) an immunoglobulin light chain or a fragment thereof comprising a VL comprising CDRs 1, 2, and 3 comprising the amino acid sequences set forth in SEQ ID NOs: 580, 581, and 582, respectively, and in some embodiments, the VL, when paired with a VH comprising the amino acid sequence set forth in SEQ ID NO: 789 binds to LILRB1 and/or LILRB2;
(41) an immunoglobulin heavy chain or a fragment thereof comprising a VH comprising CDRs 1, 2, and 3 comprising the amino acid sequences set forth in SEQ ID NOs: 607, 608, and 609, respectively, and in some embodiments, the VH, when paired with a VL comprising the amino acid sequence set forth in SEQ ID NO: 792 binds to LILRB1 and/or LILRB2;
(42) an immunoglobulin light chain or a fragment thereof comprising a VL comprising CDRs 1, 2, and 3 comprising the amino acid sequences set forth in SEQ ID NOs: 610, 611, and 612, respectively, and in some embodiments, the VL, when paired with a VH comprising the amino acid sequence set forth in SEQ ID NO: 791 binds to LILRB1 and/or LILRB2;
(43) an immunoglobulin heavy chain or a fragment thereof comprising a VH comprising CDRs 1, 2, and 3 comprising the amino acid sequences set forth in SEQ ID NOs: 637, 638, and 639, respectively, and in some embodiments, the VH, when paired with a VL comprising the amino acid sequence set forth in SEQ ID NO: 794 binds to LILRB1 and/or LILRB2;
(44) an immunoglobulin light chain or a fragment thereof comprising a VL comprising CDRs 1, 2, and 3 comprising the amino acid sequences set forth in SEQ ID NOs: 640, 641, and 642, respectively, and in some embodiments, the VL, when paired with a VH comprising the amino acid sequence set forth in SEQ ID NO: 793 binds to LILRB1 and/or LILRB2;
(45) an immunoglobulin heavy chain or a fragment thereof comprising a VH comprising CDRs 1, 2, and 3 comprising the amino acid sequences set forth in SEQ ID NOs: 667, 668, and 669, respectively, and in some embodiments, the VH, when paired with a VL comprising the amino acid sequence set forth in SEQ ID NO: 796 binds to LILRB1 and/or LILRB2;
(46) an immunoglobulin light chain or a fragment thereof comprising a VL comprising CDRs 1, 2, and 3 comprising the amino acid sequences set forth in SEQ ID NOs: 670, 671, and 672, respectively, and in some embodiments, the VL, when paired with a VH comprising the amino acid sequence set forth in SEQ ID NO: 795 binds to LILRB1 and/or LILRB2;
(47) an immunoglobulin heavy chain or a fragment thereof comprising a VH comprising CDRs 1, 2, and 3 comprising the amino acid sequences set forth in SEQ ID NOs: 697, 698, and 699, respectively, and in some embodiments, the VH, when paired with a VL comprising the amino acid sequence set forth in SEQ ID NO: 798 binds to LILRB1 and/or LILRB2;
(48) an immunoglobulin light chain or a fragment thereof comprising a VL comprising CDRs 1, 2, and 3 comprising the amino acid sequences set forth in SEQ ID NOs: 700, 701, and 702, respectively, and in some embodiments, the VL, when paired with a VH comprising the amino acid sequence set forth in SEQ ID NO: 797 binds to LILRB1 and/or LILRB2;
(49) an immunoglobulin heavy chain or a fragment thereof comprising a VH comprising CDRs 1, 2, and 3 comprising the amino acid sequences set forth in SEQ ID NOs: 727, 728, and 729, respectively, and in some embodiments, the VH, when paired with a VL comprising the amino acid sequence set forth in SEQ ID NO: 800 binds to LILRB1 and/or LILRB2;
(50) an immunoglobulin light chain or a fragment thereof comprising a VL comprising CDRs 1, 2, and 3 comprising the amino acid sequences set forth in SEQ ID NOs: 730, 731, and 732, respectively, and in some embodiments, the VL, when paired with a VH comprising the amino acid sequence set forth in SEQ ID NO: 799 binds to LILRB1 and/or LILRB2 (51) an immunoglobulin heavy chain or a fragment thereof comprising a VH comprising CDRs 1, 2, and 3 comprising the amino acid sequences set forth in SEQ ID NOs: 1069, 1070, and 1071, respectively, and wherein the VH, when paired with a VL comprising the amino acid sequence set forth in SEQ ID NO: 988 binds to LILRB1 and/or LILRB2;
(52) an immunoglobulin light chain or a fragment thereof comprising a VL comprising CDRs 1, 2, and 3 comprising the amino acid sequences set forth in SEQ ID NOs: 1072, 1073, and 1074, respectively, and wherein the VL, when paired with a VH comprising the amino acid sequence set forth in SEQ ID NO: 987 binds to LILRB1 and/or LILRB2;
(53) an immunoglobulin heavy chain or a fragment thereof comprising a VH comprising CDRs 1, 2, and 3 comprising the amino acid sequences set forth in SEQ ID NOs: 1093, 1094, and 1095, respectively, and wherein the VH, when paired with a VL comprising the amino acid sequence set forth in SEQ ID NO: 988 binds to LILRB1 and/or LILRB2;
(54) an immunoglobulin light chain or a fragment thereof comprising a VL comprising CDRs 1, 2, and 3 comprising the amino acid sequences set forth in SEQ ID NOs: 1096, 1097, and 1098, respectively, and wherein the VL, when paired with a VH comprising the amino acid sequence set forth in SEQ ID NO: 1687 binds to LILRB1 and/or LILRB2;
(55) an immunoglobulin heavy chain or a fragment thereof comprising a VH comprising CDRs 1, 2, and 3 comprising the amino acid sequences set forth in SEQ ID NOs: 1117, 1118, and 1119, respectively, and wherein the VH, when paired with a VL comprising the amino acid sequence set forth in SEQ ID NO: 988 binds to LILRB1 and/or LILRB2;
(56) an immunoglobulin light chain or a fragment thereof comprising a VL comprising CDRs 1, 2, and 3 comprising the amino acid sequences set forth in SEQ ID NOs: 1120, 1121, and 1122, respectively, and wherein the VL, when paired with a VH comprising the amino acid sequence set forth in SEQ ID NO: 1688 binds to LILRB1 and/or LILRB2;
(57) an immunoglobulin heavy chain or a fragment thereof comprising a VH comprising CDRs 1, 2, and 3 comprising the amino acid sequences set forth in SEQ ID NOs: 1141, 1142, and 1143, respectively, and wherein the VH, when paired with a VL comprising the amino acid sequence set forth in SEQ ID NO: 989 binds to LILRB1 and/or LILRB2;
(58) an immunoglobulin light chain or a fragment thereof comprising a VL comprising CDRs 1, 2, and 3 comprising the amino acid sequences set forth in SEQ ID NOs: 1144, 1145, and 1146, respectively, and wherein the VL, when paired with a VH comprising the amino acid sequence set forth in SEQ ID NO: 987 binds to LILRB1 and/or LILRB2;
(59) an immunoglobulin heavy chain or a fragment thereof comprising a VH comprising CDRs 1, 2, and 3 comprising the amino acid sequences set forth in SEQ ID NOs: 1165, 1166, and 1167, respectively, and wherein the VH, when paired with a VL comprising the amino acid sequence set forth in SEQ ID NO: 990 binds to LILRB1 and/or LILRB2;
(60) an immunoglobulin light chain or a fragment thereof comprising a VL comprising CDRs 1, 2, and 3 comprising the amino acid sequences set forth in SEQ ID NOs: 1168, 1169, and 1170, respectively, and wherein the VL, when paired with a VH comprising the amino acid sequence set forth in SEQ ID NO: 987 binds to LILRB1 and/or LILRB2;
(61) an immunoglobulin heavy chain or a fragment thereof comprising a VH comprising CDRs 1, 2, and 3 comprising the amino acid sequences set forth in SEQ ID NOs: 1189, 1190, and 1191, respectively, and wherein the VH, when paired with a VL comprising the amino acid sequence set forth in SEQ ID NO: 991 binds to LILRB1 and/or LILRB2;
(62) an immunoglobulin light chain or a fragment thereof comprising a VL comprising CDRs 1, 2, and 3 comprising the amino acid sequences set forth in SEQ ID NOs: 1192, 1193, and 1194, respectively, and wherein the VL, when paired with a VH comprising the amino acid sequence set forth in SEQ ID NO: 987 binds to LILRB1 and/or LILRB2;
(63) an immunoglobulin heavy chain or a fragment thereof comprising a VH comprising CDRs 1, 2, and 3 comprising the amino acid sequences set forth in SEQ ID NOs: 1213, 1214, and 1215, respectively, and wherein the VH, when paired with a VL comprising the amino acid sequence set forth in SEQ ID NO: 992 binds to LILRB1 and/or LILRB2;
(64) an immunoglobulin light chain or a fragment thereof comprising a VL comprising CDRs 1, 2, and 3 comprising the amino acid sequences set forth in SEQ ID NOs: 1216, 1217, and 1218, respectively, and wherein the VL, when paired with a VH comprising the amino acid sequence set forth in SEQ ID NO: 987 binds to LILRB1 and/or LILRB2;
(65) an immunoglobulin heavy chain or a fragment thereof comprising a VH comprising CDRs 1, 2, and 3 comprising the amino acid sequences set forth in SEQ ID NOs: 1237, 1238, and 1239, respectively, and wherein the VH, when paired with a VL comprising the amino acid sequence set forth in SEQ ID NO: 1020 binds to LILRB1 and/or LILRB2;
(66) an immunoglobulin light chain or a fragment thereof comprising a VL comprising CDRs 1, 2, and 3 comprising the amino acid sequences set forth in SEQ ID NOs: 1240, 1241, and 1242, respectively, and wherein the VL, when paired with a VH comprising the amino acid sequence set forth in SEQ ID NO: 1019 binds to LILRB1 and/or LILRB2;
(67) an immunoglobulin heavy chain or a fragment thereof comprising a VH comprising CDRs 1, 2, and 3 comprising the amino acid sequences set forth in SEQ ID NOs: 1261, 1262, and 1263, respectively, and wherein the VH, when paired with a VL comprising the amino acid sequence set forth in SEQ ID NO: 1022 binds to LILRB1 and/or LILRB2;
(68) an immunoglobulin light chain or a fragment thereof comprising a VL comprising CDRs 1, 2, and 3 comprising the amino acid sequences set forth in SEQ ID NOs: 1264, 1265, and 1266, respectively, and wherein the VL, when paired with a VH comprising the amino acid sequence set forth in SEQ ID NO: 1021 binds to LILRB1 and/or LILRB2;
(69) an immunoglobulin heavy chain or a fragment thereof comprising a VH comprising CDRs 1, 2, and 3 comprising the amino acid sequences set forth in SEQ ID NOs: 1285, 1286, and 1287, respectively, and wherein the VH, when paired with a VL comprising the amino acid sequence set forth in SEQ ID NO: 1024 binds to LILRB1 and/or LILRB2;
(70) an immunoglobulin light chain or a fragment thereof comprising a VL comprising CDRs 1, 2, and 3 comprising the amino acid sequences set forth in SEQ ID NOs: 1288, 1289, and 1290, respectively, and wherein the VL, when paired with a VH comprising the amino acid sequence set forth in SEQ ID NO: 1023 binds to LILRB1 and/or LILRB2;
(71) an immunoglobulin heavy chain or a fragment thereof comprising a VH comprising CDRs 1, 2, and 3 comprising the amino acid sequences set forth in SEQ ID NOs: 1309, 1310, and 1311, respectively, and wherein the VH, when paired with a VL comprising the amino acid sequence set forth in SEQ ID NO: 1026 binds to LILRB1 and/or LILRB2;
(72) an immunoglobulin light chain or a fragment thereof comprising a VL comprising CDRs 1, 2, and 3 comprising the amino acid sequences set forth in SEQ ID NOs: 1312, 1313, and 1314, respectively, and wherein the VL, when paired with a VH comprising the amino acid sequence set forth in SEQ ID NO: 1025 binds to LILRB1 and/or LILRB2;
(73) an immunoglobulin heavy chain or a fragment thereof comprising a VH comprising CDRs 1, 2, and 3 comprising the amino acid sequences set forth in SEQ ID NOs: 1333, 1334, and 1335, respectively, and wherein the VH, when paired with a VL comprising the amino acid sequence set forth in SEQ ID NO: 1028 binds to LILRB1 and/or LILRB2;
(74) an immunoglobulin light chain or a fragment thereof comprising a VL comprising CDRs 1, 2, and 3 comprising the amino acid sequences set forth in SEQ ID NOs: 1336, 1337, and 1338, respectively, and wherein the VL, when paired with a VH comprising the amino acid sequence set forth in SEQ ID NO: 1027 binds to LILRB1 and/or LILRB2;
(75) an immunoglobulin heavy chain or a fragment thereof comprising a VH comprising CDRs 1, 2, and 3 comprising the amino acid sequences set forth in SEQ ID NOs: 1357, 1358, and 1359, respectively, and wherein the VH, when paired with a VL comprising the amino acid sequence set forth in SEQ ID NO: 1030 binds to LILRB1 and/or LILRB2;
(76) an immunoglobulin light chain or a fragment thereof comprising a VL comprising CDRs 1, 2, and 3 comprising the amino acid sequences set forth in SEQ ID NOs: 1360, 1361, and 1362, respectively, and wherein the VL, when paired with a VH comprising the amino acid sequence set forth in SEQ ID NO: 1029 binds to LILRB1 and/or LILRB2;
(77) an immunoglobulin heavy chain or a fragment thereof comprising a VH comprising CDRs 1, 2, and 3 comprising the amino acid sequences set forth in SEQ ID NOs: 1381, 1382, and 1383, respectively, and wherein the VH, when paired with a VL comprising the amino acid sequence set forth in SEQ ID NO: 1032 binds to LILRB1 and/or LILRB2;
(78) an immunoglobulin light chain or a fragment thereof comprising a VL comprising CDRs 1, 2, and 3 comprising the amino acid sequences set forth in SEQ ID NOs: 1384, 1385, and 1386, respectively, and wherein the VL, when paired with a VH comprising the amino acid sequence set forth in SEQ ID NO: 1031 binds to LILRB1 and/or LILRB2;
(79) an immunoglobulin heavy chain or a fragment thereof comprising a VH comprising CDRs 1, 2, and 3 comprising the amino acid sequences set forth in SEQ ID NOs: 1405, 1406, and 1407, respectively, and wherein the VH, when paired with a VL comprising the amino acid sequence set forth in SEQ ID NO: 1034 binds to LILRB1 and/or LILRB2;
(80) an immunoglobulin light chain or a fragment thereof comprising a VL comprising CDRs 1, 2, and 3 comprising the amino acid sequences set forth in SEQ ID NOs: 1408, 1409, and 1410, respectively, and wherein the VL, when paired with a VH comprising the amino acid sequence set forth in SEQ ID NO: 1033 binds to LILRB1 and/or LILRB2;
(81) an immunoglobulin heavy chain or a fragment thereof comprising a VH comprising CDRs 1, 2, and 3 comprising the amino acid sequences set forth in SEQ ID NOs: 1429, 1430, and 1431, respectively, and wherein the VH, when paired with a VL comprising the amino acid sequence set forth in SEQ ID NO: 1036 binds to LILRB1 and/or LILRB2;
(82) an immunoglobulin light chain or a fragment thereof comprising a VL comprising CDRs 1, 2, and 3 comprising the amino acid sequences set forth in SEQ ID NOs: 1432, 1433, and 1434, respectively, and wherein the VL, when paired with a VH comprising the amino acid sequence set forth in SEQ ID NO: 1035 binds to LILRB1 and/or LILRB2;
(83) an immunoglobulin heavy chain or a fragment thereof comprising a VH comprising CDRs 1, 2, and 3 comprising the amino acid sequences set forth in SEQ ID NOs: 1453, 1454, and 1455, respectively, and wherein the VH, when paired with a VL comprising the amino acid sequence set forth in SEQ ID NO: 1038 binds to LILRB1 and/or LILRB2;
(84) an immunoglobulin light chain or a fragment thereof comprising a VL comprising CDRs 1, 2, and 3 comprising the amino acid sequences set forth in SEQ ID NOs: 1456, 1457, and 1458, respectively, and wherein the VL, when paired with a VH comprising the amino acid sequence set forth in SEQ ID NO: 1037 binds to LILRB1 and/or LILRB2;
(85) an immunoglobulin heavy chain or a fragment thereof comprising a VH comprising CDRs 1, 2, and 3 comprising the amino acid sequences set forth in SEQ ID NOs: 1477, 1478, and 1479, respectively, and wherein the VH, when paired with a VL comprising the amino acid sequence set forth in SEQ ID NO: 1040 binds to LILRB1 and/or LILRB2;
(86) an immunoglobulin light chain or a fragment thereof comprising a VL comprising CDRs 1, 2, and 3 comprising the amino acid sequences set forth in SEQ ID NOs: 1480, 1481, and 1482, respectively, and wherein the VL, when paired with a VH comprising the amino acid sequence set forth in SEQ ID NO: 1039 binds to LILRB1 and/or LILRB2;
(87) an immunoglobulin heavy chain or a fragment thereof comprising a VH comprising CDRs 1, 2, and 3 comprising the amino acid sequences set forth in SEQ ID NOs: 1501, 1502, and 1503, respectively, and wherein the VH, when paired with a VL comprising the amino acid sequence set forth in SEQ ID NO: 1042 binds to LILRB1 and/or LILRB2;
(88) an immunoglobulin light chain or a fragment thereof comprising a VL comprising CDRs 1, 2, and 3 comprising the amino acid sequences set forth in SEQ ID NOs: 1504, 1505, and 1506, respectively, and wherein the VL, when paired with a VH comprising the amino acid sequence set forth in SEQ ID NO: 1041 binds to LILRB1 and/or LILRB2;
(89) an immunoglobulin heavy chain or a fragment thereof comprising a VH comprising CDRs 1, 2, and 3 comprising the amino acid sequences set forth in SEQ ID NOs: 1525, 1526, and 1527, respectively, and wherein the VH, when paired with a VL comprising the amino acid sequence set forth in SEQ ID NO: 1044, 1689, or 1690 binds to LILRB1 and/or LILRB2;
(90) an immunoglobulin light chain or a fragment thereof comprising a VL comprising CDRs 1, 2, and 3 comprising the amino acid sequences set forth in SEQ ID NOs: 1528, 1529, and 1530, respectively, and wherein the VL, when paired with a VH comprising the amino acid sequence set forth in SEQ ID NO: 1043 binds to LILRB1 and/or LILRB2;
(91) an immunoglobulin heavy chain or a fragment thereof comprising a VH comprising CDRs 1, 2, and 3 comprising the amino acid sequences set forth in SEQ ID NOs: 1549, 1550, and 1551, respectively, and wherein the VH, when paired with a VL comprising the amino acid sequence set forth in SEQ ID NO: 1046 binds to LILRB1 and/or LILRB2;
(92) an immunoglobulin light chain or a fragment thereof comprising a VL comprising CDRs 1, 2, and 3 comprising the amino acid sequences set forth in SEQ ID NOs: 1552, 1553, and 1554, respectively, and wherein the VL, when paired with a VH comprising the amino acid sequence set forth in SEQ ID NO: 1045 binds to LILRB1 and/or LILRB2;
(93) an immunoglobulin heavy chain or a fragment thereof comprising a VH comprising CDRs 1, 2, and 3 comprising the amino acid sequences set forth in SEQ ID NOs: 1573, 1574, and 1575, respectively, and wherein the VH, when paired with a VL comprising the amino acid sequence set forth in SEQ ID NO: 1048 binds to LILRB1 and/or LILRB2;
(94) an immunoglobulin light chain or a fragment thereof comprising a VL comprising CDRs 1, 2, and 3 comprising the amino acid sequences set forth in SEQ ID NOs: 1576, 1577, and 1578, respectively, and wherein the VL, when paired with a VH comprising the amino acid sequence set forth in SEQ ID NO: 1047 binds to LILRB1 and/or LILRB2;
(95) an immunoglobulin heavy chain or a fragment thereof comprising a VH comprising CDRs 1, 2, and 3 comprising the amino acid sequences set forth in SEQ ID NOs: 1597, 1598, and 1599, respectively, and wherein the VH, when paired with a VL comprising the amino acid sequence set forth in SEQ ID NO: 1050 binds to LILRB1 and/or LILRB2;
(96) an immunoglobulin light chain or a fragment thereof comprising a VL comprising CDRs 1, 2, and 3 comprising the amino acid sequences set forth in SEQ ID NOs: 1600, 1601, and 1602, respectively, and wherein the VL, when paired with a VH comprising the amino acid sequence set forth in SEQ ID NO: 1049 binds to LILRB1 and/or LILRB2;
(97) an immunoglobulin heavy chain or a fragment thereof comprising a VH comprising CDRs 1, 2, and 3 comprising the amino acid sequences set forth in SEQ ID NOs: 1621, 1622, and 1623, respectively, and wherein the VH, when paired with a VL comprising the amino acid sequence set forth in SEQ ID NO: 1052 binds to LILRB1 and/or LILRB2;
(98) an immunoglobulin light chain or a fragment thereof comprising a VL comprising CDRs 1, 2, and 3 comprising the amino acid sequences set forth in SEQ ID NOs: 1624, 1625, and 1626, respectively, and wherein the VL, when paired with a VH comprising the amino acid sequence set forth in SEQ ID NO: 1051 binds to LILRB1 and/or LILRB2;
(99) an immunoglobulin heavy chain or a fragment thereof comprising a VH comprising CDRs 1, 2, and 3 comprising the amino acid sequences set forth in SEQ ID NOs: 1645, 1646, and 1647, respectively, and wherein the VH, when paired with a VL comprising the amino acid sequence set forth in SEQ ID NO: 1054 binds to LILRB1 and/or LILRB2;
(100) an immunoglobulin light chain or a fragment thereof comprising a VL comprising CDRs 1, 2, and 3 comprising the amino acid sequences set forth in SEQ ID NOs: 1648, 1649, and 1650, respectively, and wherein the VL, when paired with a VH comprising the amino acid sequence set forth in SEQ ID NO: 1053 binds to LILRB1 and/or LILRB2;
(101) an immunoglobulin heavy chain or a fragment thereof comprising a VH comprising CDRs 1, 2, and 3 comprising the amino acid sequences set forth in SEQ ID NOs: 1669, 1670, and 1671, respectively, and wherein the VH, when paired with a VL comprising the amino acid sequence set forth in SEQ ID NO: 1056 binds to LILRB1 and/or LILRB2; or
(102) an immunoglobulin light chain or a fragment thereof comprising a VL comprising CDRs 1, 2, and 3 comprising the amino acid sequences set forth in SEQ ID NOs: 1672, 1673, and 1674, respectively, and wherein the VL, when paired with a VH comprising the amino acid sequence set forth in SEQ ID NO: 1055 binds to LILRB1 and/or LILRB2.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 7, 8, and 9, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 10, 11, and 12, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 37, 38, and 39, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 40, 41, and 42, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 67, 68, and 69, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 70, 71, and 72, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 97, 98, and 99, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 100, 101, and 102, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 127, 128, and 129, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 130, 131, and 132, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 157, 158, and 159, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 160, 161, and 162, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 187, 188, and 189, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 190, 191, and 192, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 217, 218, and 219, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 220, 221, and 222, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 247, 248, and 249, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 250, 251, and 252, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 277, 278, and 279 respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 280, 281, and 282, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 307, 308, and 309, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 310, 311, and 312, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 337, 338, and 339, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 340, 341, and 342, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 367, 368, and 369, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 370, 371, and 372, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 397, 398, and 399, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 400, 401, and 402, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 427, 428, and 429, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 430, 431, and 432, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 457, 458, and 459, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 460, 461, and 462, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 487, 488, and 489, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 490, 491, and 492, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 517, 518, and 519, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 520, 521, and 522, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 547, 548, and 549, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 550, 551, and 552, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 577, 578, and 579, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 580, 581, and 582, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 607, 608, and 609, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 610, 611, and 612, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 637, 638, and 639, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 640, 641, and 642, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 667, 668, and 669, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 700, 701, and 702, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 697, 698, and 699, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 700, 701, and 702, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 727, 728, and 729, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 730, 731, and 732, respectively. In some embodiments the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1069, 1070, and 1071, respectively. In some embodiments the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1072, 1073, and 1074, respectively. In some embodiments the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1093, 1094, and 1095, respectively. In some embodiments the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1096, 1097, and 1098, respectively. In some embodiments the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1117, 1118, and 1119, respectively. In some embodiments the nucleic acid comprises a polynucleotide encoding a polypeptide comprising an immunoglobulin light chain or a fragment thereof comprising a VL comprising CDRS 1, 2, and 3 comprising the amino acid sequences set forth in SEQ ID NOs: 1120, 1121, and 1122, respectively. In some embodiments the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1141, 1142, and 1143, respectively. In some embodiments the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1144, 1145, and 1146, respectively. In some embodiments the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1165, 1166, and 1167, respectively. In some embodiments the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1168, 1169, and 1170, respectively. In some embodiments the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1189, 1190, and 1191, respectively. In some embodiments the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1192, 1193, and 1194, respectively. In some embodiments the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1213, 1214, and 1215, respectively. In some embodiments the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1216, 1217, and 1218, respectively. In some embodiments the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1237, 1238, and 1239, respectively. In some embodiments the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1240, 1241, and 1242, respectively. In some embodiments the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1261, 1262, and 1263, respectively. In some embodiments the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1264, 1265, and 1266, respectively. In some embodiments the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1285, 1286, and 1287, respectively. In some embodiments the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1288, 1289, and 1290, respectively. In some embodiments the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1309, 1310, and 1311, respectively. In some embodiments the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1312, 1313, and 1314, respectively. In some embodiments the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1333, 1334, and 1335, respectively. In some embodiments the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1336, 1337, and 1338, respectively. In some embodiments the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1357, 1358, and 1359, respectively. In some embodiments the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1360, 1361, and 1362, respectively. In some embodiments the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1381, 1382, and 1383, respectively. In some embodiments the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1384, 1385, and 1386, respectively. In some embodiments the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1405, 1406, and 1407, respectively. In some embodiments the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1408, 1409, and 1410, respectively. In some embodiments the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1429, 1430, and 1431, respectively. In some embodiments the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1432, 1433, and 1434, respectively. In some embodiments the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1453, 1454, and 1455, respectively. In some embodiments the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1456, 1457, and 1458, respectively. In some embodiments the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1477, 1478, and 1479, respectively. In some embodiments the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1480, 1481, and 1482, respectively. In some embodiments the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1501, 1502, and 1503, respectively. In some embodiments the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1504, 1505, and 1506, respectively. In some embodiments the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1525, 1526, and 1527, respectively. In some embodiments the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1528, 1529, and 1530, respectively. In some embodiments the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1549, 1550, and 1551, respectively. In some embodiments the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1552, 1553, and 1554, respectively. In some embodiments the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1573, 1574, and 1575, respectively. In some embodiments the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1576, 1577, and 1578, respectively. In some embodiments the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1597, 1598, and 1599, respectively. In some embodiments the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1600, 1601, and 1602, respectively. In some embodiments the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1621, 1622, and 1623, respectively. In some embodiments the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1624, 1625, and 1626, respectively. In some embodiments the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1645, 1646, and 1647, respectively. In some embodiments the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1648, 1649, and 1650, respectively. In some embodiments the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1669, 1670, and 1671, respectively. In some embodiments the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1672, 1673, and 1674, respectively.

In some embodiments, the VH when paired with a VL specifically binds to human LILRB1 and/or LILRB2; or the VL when paired with a VH specifically binds to human LILRB1 and/or LILRB2. In some embodiments, the immunoglobulin heavy chain or the fragment thereof is a humanized immunoglobulin heavy chain or a fragment thereof, and the immunoglobulin light chain or the fragment thereof is a humanized immunoglobulin light chain or a fragment thereof. In some embodiments, the nucleic acid encodes a single-chain variable fragment (scFv) or a multi-specific antibody (e.g., a bispecific antibody). In some embodiments, the nucleic acid is cDNA.

In one aspect, the disclosure is related to a vector comprising one or more of the nucleic acids as described herein. In one aspect, the disclosure is related to vector comprising two of the nucleic acids as described herein, in some embodiments, the vector encodes the VL region and the VH region that together bind to LILRB1 and/or LILRB2. In one aspect, the disclosure is related to a pair of vectors, in some embodiments, each vector comprises one of the nucleic acids as described herein, in some embodiments, together the pair of vectors encodes the VL region and the VH region that together bind to LILRB1 and/or LILRB2.

In one aspect, the disclosure is related to a cell comprising the vector or the pair of vectors as described herein. In some embodiments, the cell is a CHO cell. In one aspect, the disclosure is related to a cell comprising one or more of the nucleic acids as described herein. In one aspect, the disclosure is related to a cell comprising two of the nucleic acids as described herein. In some embodiments, the two nucleic acids together encode the VL region and the VH region that together bind to LILRB1 and/or LILRB2.

In one aspect, the disclosure is related to a method of producing an antibody or an antigen-binding fragment thereof, the method comprising (a) culturing the cell as described herein under conditions sufficient for the cell to produce the antibody or the antigen-binding fragment; and (b) collecting the antibody or the antigen-binding fragment produced by the cell.

In one aspect, the disclosure is related to an antibody or antigen-binding fragment thereof that binds to LILRB1 and/or LILRB2 comprising a heavy chain variable region (VH) comprising an amino acid sequence that is at least 90% identical to a selected VH sequence, and a light chain variable region (VL) comprising an amino acid sequence that is at least 90% identical to a selected VL sequence, in some embodiments, the selected VH sequence and the selected VL sequence are one of the following: (1) the selected VH sequence is SEQ ID NO: 751, and the selected VL sequence is SEQ ID NO: 752; (2) the selected VH sequence is SEQ ID NO: 753, and the selected VL sequence is SEQ ID NO: 754; (3) the selected VH sequence is SEQ ID NO: 755, and the selected VL sequence is SEQ ID NO: 756; (4) the selected VH sequence is SEQ ID NO: 757, and the selected VL sequence is SEQ ID NO: 758; (5) the selected VH sequence is SEQ ID NO: 759, and the selected VL sequence is SEQ ID NO: 760; (6) the selected VH sequence is SEQ ID NO: 761, and the selected VL sequence is SEQ ID NO: 762; (7) the selected VH sequence is SEQ ID NO: 763, and the selected VL sequence is SEQ ID NO: 764; (8) the selected VH sequence is SEQ ID NO: 765, and the selected VL sequence is SEQ ID NO: 766; (9) the selected VH sequence is SEQ ID NO: 767, and the selected VL sequence is SEQ ID NO: 768; (10) the selected VH sequence is SEQ ID NO: 769, and the selected VL sequence is SEQ ID NO: 770; (11) the selected VH sequence is SEQ ID NO: 771, and the selected VL sequence is SEQ ID NO: 772; (12) the selected VH sequence is SEQ ID NO: 773, and the selected VL sequence is SEQ ID NO: 774; (13) the selected VH sequence is SEQ ID NO: 775, and the selected VL sequence is SEQ ID NO: 776; (14) the selected VH sequence is SEQ ID NO: 777, and the selected VL sequence is SEQ ID NO: 778; (15) the selected VH sequence is SEQ ID NO: 779, and the selected VL sequence is SEQ ID NO: 780; (16) the selected VH sequence is SEQ ID NO: 781, and the selected VL sequence is SEQ ID NO: 782; (17) the selected VH sequence is SEQ ID NO: 783, and the selected VL sequence is SEQ ID NO: 784; (18) the selected VH sequence is SEQ ID NO: 785, and the selected VL sequence is SEQ ID NO: 786; (19) the selected VH sequence is SEQ ID NO: 787, and the selected VL sequence is SEQ ID NO: 788; (20) the selected VH sequence is SEQ ID NO: 789, and the selected VL sequence is SEQ ID NO: 790; (21) the selected VH sequence is SEQ ID NO: 791, and the selected VL sequence is SEQ ID NO: 792; (22) the selected VH sequence is SEQ ID NO: 793, and the selected VL sequence is SEQ ID NO: 794; (23) the selected VH sequence is SEQ ID NO: 795, and the selected VL sequence is SEQ ID NO: 796; (24) the selected VH sequence is SEQ ID NO: 797, and the selected VL sequence is SEQ ID NO: 798; (25) the selected VH sequence is SEQ ID NO: 799, and the selected VL sequence is SEQ ID NO: 800; (26) the selected VH sequence is SEQ ID NO: 841, and the selected VL sequence is SEQ ID NO: 842; (27) the selected VH sequence is SEQ ID NO: 843, and the selected VL sequence is SEQ ID NO: 844; (28) the selected VH sequence is SEQ ID NO: 845, and the selected VL sequence is SEQ ID NO: 846; (29) the selected VH sequence is SEQ ID NO: 847, and the selected VL sequence is SEQ ID NO: 848; (30) the selected VH sequence is SEQ ID NO: 849, and the selected VL sequence is SEQ ID NO: 850; (31) the selected VH sequence is SEQ ID NO: 851, and the selected VL sequence is SEQ ID NO: 852; (32) the selected VH sequence is SEQ ID NO: 853, and the selected VL sequence is SEQ ID NO: 854; (33) the selected VH sequence is SEQ ID NO: 855, and the selected VL sequence is SEQ ID NO: 856; (34) the selected VH sequence is SEQ ID NO: 857, and the selected VL sequence is SEQ ID NO: 858; (35) the selected VH sequence is SEQ ID NO: 859, and the selected VL sequence is SEQ ID NO: 860; (36) the selected VH sequence is SEQ ID NO: 861, and the selected VL sequence is SEQ ID NO: 862; (37) the selected VH sequence is SEQ ID NO: 863, and the selected VL sequence is SEQ ID NO: 864; (38) the selected VH sequence is SEQ ID NO: 865, and the selected VL sequence is SEQ ID NO: 866; (39) the selected VH sequence is SEQ ID NO: 867, and the selected VL sequence is SEQ ID NO: 868; (40) the selected VH sequence is SEQ ID NO: 869, and the selected VL sequence is SEQ ID NO: 870; (41) the selected VH sequence is SEQ ID NO: 871, and the selected VL sequence is SEQ ID NO: 872; (42) the selected VH sequence is SEQ ID NO: 873, and the selected VL sequence is SEQ ID NO: 874; (43) the selected VH sequence is SEQ ID NO: 875, and the selected VL sequence is SEQ ID NO: 876; (44) the selected VH sequence is SEQ ID NO: 877, and the selected VL sequence is SEQ ID NO: 878; (45) the selected VH sequence is SEQ ID NO: 879, and the selected VL sequence is SEQ ID NO: 880; (46) the selected VH sequence is SEQ ID NO: 881, and the selected VL sequence is SEQ ID NO: 882; (47) the selected VH sequence is SEQ ID NO: 883, and the selected VL sequence is SEQ ID NO: 884; (48) the selected VH sequence is SEQ ID NO: 885, and the selected VL sequence is SEQ ID NO: 886; (49) the selected VH sequence is SEQ ID NO: 887, and the selected VL sequence is SEQ ID NO: 888; (50) the selected VH sequence is SEQ ID NO: 889, and the selected VL sequence is SEQ ID NO: 890; (51) the selected VH sequence is SEQ ID NO: 891, and the selected VL sequence is SEQ ID NO: 892; (52) the selected VH sequence is SEQ ID NO: 893, and the selected VL sequence is SEQ ID NO: 894; (53) the selected VH sequence is SEQ ID NO: 895, and the selected VL sequence is SEQ ID NO: 896; (54) the selected VH sequence is SEQ ID NO: 897, and the selected VL sequence is SEQ ID NO: 898; (55) the selected VH sequence is SEQ ID NO: 899, and the selected VL sequence is SEQ ID NO: 900; (56) the selected VH sequence is SEQ ID NO: 901, and the selected VL sequence is SEQ ID NO: 902; (57) the selected VH sequence is SEQ ID NO: 903, and the selected VL sequence is SEQ ID NO: 904; (58) the selected VH sequence is SEQ ID NO: 905, and the selected VL sequence is SEQ ID NO: 906; (59) the selected VH sequence is SEQ ID NO: 907, and the selected VL sequence is SEQ ID NO: 908; (60) the selected VH sequence is SEQ ID NO: 909, and the selected VL sequence is SEQ ID NO: 910; (61) the selected VH sequence is SEQ ID NO: 911, and the selected VL sequence is SEQ ID NO: 912; (62) the selected VH sequence is SEQ ID NO: 913, and the selected VL sequence is SEQ ID NO: 914; (63) the selected VH sequence is SEQ ID NO: 915, and the selected VL sequence is SEQ ID NO: 916; (64) the selected VH sequence is SEQ ID NO: 917, and the selected VL sequence is SEQ ID NO: 918; (65) the selected VH sequence is SEQ ID NO: 919, and the selected VL sequence is SEQ ID NO: 920; (66) the selected VH sequence is SEQ ID NO: 921, and the selected VL sequence is SEQ ID NO: 922; (67) the selected VH sequence is SEQ ID NO: 923, and the selected VL sequence is SEQ ID NO: 924; (68) the selected VH sequence is SEQ ID NO: 925, and the selected VL sequence is SEQ ID NO: 926; (69) the selected VH sequence is SEQ ID NO: 927, and the selected VL sequence is SEQ ID NO: 928; (70) the selected VH sequence is SEQ ID NO: 929, and the selected VL sequence is SEQ ID NO: 930; (71) the selected VH sequence is SEQ ID NO: 931, and the selected VL sequence is SEQ ID NO: 932; (72) the selected VH sequence is SEQ ID NO: 933, and the selected VL sequence is SEQ ID NO: 934; (73) the selected VH sequence is SEQ ID NO: 935, and the selected VL sequence is SEQ ID NO: 936; (74) the selected VH sequence is SEQ ID NO: 937, and the selected VL sequence is SEQ ID NO: 938; (75) the selected VH sequence is SEQ ID NO: 939, and the selected VL sequence is SEQ ID NO: 940; (76) the selected VH sequence is SEQ ID NO: 941, and the selected VL sequence is SEQ ID NO: 942; (77) the selected VH sequence is SEQ ID NO: 943, and the selected VL sequence is SEQ ID NO: 944; (78) the selected VH sequence is SEQ ID NO: 945, and the selected VL sequence is SEQ ID NO: 946; (79) the selected VH sequence is SEQ ID NO: 947, and the selected VL sequence is SEQ ID NO: 948; (80) the selected VH sequence is SEQ ID NO: 949, and the selected VL sequence is SEQ ID NO: 950; (81) the selected VH sequence is SEQ ID NO: 951, and the selected VL sequence is SEQ ID NO: 952; (82) the selected VH sequence is SEQ ID NO: 953, and the selected VL sequence is SEQ ID NO: 954; (83) the selected VH sequence is SEQ ID NO: 955, and the selected VL sequence is SEQ ID NO: 956; (84) the selected VH sequence is SEQ ID NO: 957, and the selected VL sequence is SEQ ID NO: 958; (85) the selected VH sequence is SEQ ID NO: 959, and the selected VL sequence is SEQ ID NO: 960; (86) the selected VH sequence is SEQ ID NO: 961, and the selected VL sequence is SEQ ID NO: 962; (87) the selected VH sequence is SEQ ID NO: 963, and the selected VL sequence is SEQ ID NO: 964; (88) the selected VH sequence is SEQ ID NO: 965, and the selected VL sequence is SEQ ID NO: 966; (89) the selected VH sequence is SEQ ID NO: 967, and the selected VL sequence is SEQ ID NO: 968; (90) the selected VH sequence is SEQ ID NO: 969, and the selected VL sequence is SEQ ID NO: 970; (91) the selected VH sequence is SEQ ID NO: 971, and the selected VL sequence is SEQ ID NO: 972; (92) the selected VH sequence is SEQ ID NO: 973, and the selected VL sequence is SEQ ID NO: 974; (93) the selected VH sequence is SEQ ID NO: 975, and the selected VL sequence is SEQ ID NO: 976; (94) the selected VH sequence is SEQ ID NO: 977, and the selected VL sequence is SEQ ID NO: 978; (95) the selected VH sequence is SEQ ID NO: 979, and the selected VL sequence is SEQ ID NO: 980, 981, 982, 983, or 984; (96) the selected VH sequence is SEQ ID NO: 985, and the selected VL sequence is SEQ ID NO: 986; (97) the selected VH sequence is SEQ ID NO: 987, 1687, or 1688, and the selected VL sequence is SEQ ID NO: 988, 989, 990, 991, or 992; (98) the selected VH sequence is SEQ ID NO: 993, and the selected VL sequence is SEQ ID NO: 994; (99) the selected VH sequence is SEQ ID NO: 995, and the selected VL sequence is SEQ ID NO: 996; (100) the selected VH sequence is SEQ ID NO: 997, and the selected VL sequence is SEQ ID NO: 998; (101) the selected VH sequence is SEQ ID NO: 999, and the selected VL sequence is SEQ ID NO: 1000, 1001, 1002, 1003, or 1004; (102) the selected VH sequence is SEQ ID NO: 1005, and the selected VL sequence is SEQ ID NO: 1006; (103) the selected VH sequence is SEQ ID NO: 1007, and the selected VL sequence is SEQ ID NO: 1008, 1009, or 1010; (104) the selected VH sequence is SEQ ID NO: 1011, and the selected VL sequence is SEQ ID NO: 1012; (105) the selected VH sequence is SEQ ID NO: 1013, and the selected VL sequence is SEQ ID NO: 1014; (106) the selected VH sequence is SEQ ID NO: 1015, and the selected VL sequence is SEQ ID NO: 1016; (107) the selected VH sequence is SEQ ID NO: 1017, and the selected VL sequence is SEQ ID NO: 1018; (108) the selected VH sequence is SEQ ID NO: 1019, and the selected VL sequence is SEQ ID NO: 1020; (109) the selected VH sequence is SEQ ID NO: 1021, and the selected VL sequence is SEQ ID NO: 1022; (110) the selected VH sequence is SEQ ID NO: 1023, and the selected VL sequence is SEQ ID NO: 1024; (111) the selected VH sequence is SEQ ID NO: 1025, and the selected VL sequence is SEQ ID NO: 1026; (112) the selected VH sequence is SEQ ID NO: 1027, and the selected VL sequence is SEQ ID NO: 1028; (113) the selected VH sequence is SEQ ID NO: 1029, and the selected VL sequence is SEQ ID NO: 1030; (114) the selected VH sequence is SEQ ID NO: 1031, and the selected VL sequence is SEQ ID NO: 1032; (115) the selected VH sequence is SEQ ID NO: 1033, and the selected VL sequence is SEQ ID NO: 1034; (116) the selected VH sequence is SEQ ID NO: 1035, and the selected VL sequence is SEQ ID NO: 1036; (117) the selected VH sequence is SEQ ID NO: 1037, and the selected VL sequence is SEQ ID NO: 1038; (118) the selected VH sequence is SEQ ID NO: 1039, and the selected VL sequence is SEQ ID NO: 1040; (119) the selected VH sequence is SEQ ID NO: 1041, and the selected VL sequence is SEQ ID NO: 1042; (120) the selected VH sequence is SEQ ID NO: 1043, and the selected VL sequence is SEQ ID NO: 1044, 1689, or 1690; (121) the selected VH sequence is SEQ ID NO: 1045, and the selected VL sequence is SEQ ID NO: 1046; (122) the selected VH sequence is SEQ ID NO: 1047, and the selected VL sequence is SEQ ID NO: 1048; (123) the selected VH sequence is SEQ ID NO: 1049, and the selected VL sequence is SEQ ID NO: 1050; (124) the selected VH sequence is SEQ ID NO: 1051, and the selected VL sequence is SEQ ID NO: 1052; (125) the selected VH sequence is SEQ ID NO: 1053, and the selected VL sequence is SEQ ID NO: 1054; and (126) the selected VH sequence is SEQ ID NO: 1055, and the selected VL sequence is SEQ ID NO: 1056.

In some embodiments, the VH comprises the sequence of SEQ ID NO: 751 and the VL comprises the sequence of SEQ ID NO: 752. In some embodiments, the VH comprises the sequence of SEQ ID NO: 753 and the VL comprises the sequence of SEQ ID NO: 754. In some embodiments, the VH comprises the sequence of SEQ ID NO: 755 and the VL comprises the sequence of SEQ ID NO: 756. In some embodiments, the VH comprises the sequence of SEQ ID NO: 757 and the VL comprises the sequence of SEQ ID NO: 758. In some embodiments, the VH comprises the sequence of SEQ ID NO: 759 and the VL comprises the sequence of SEQ ID NO: 760. In some embodiments, the VH comprises the sequence of SEQ ID NO: 761 and the VL comprises the sequence of SEQ ID NO: 762. In some embodiments, the VH comprises the sequence of SEQ ID NO: 763 and the VL comprises the sequence of SEQ ID NO: 764. In some embodiments, the VH comprises the sequence of SEQ ID NO: 765 and the VL comprises the sequence of SEQ ID NO: 766. In some embodiments, the VH comprises the sequence of SEQ ID NO: 767 and the VL comprises the sequence of SEQ ID NO: 768. In some embodiments, the VH comprises the sequence of SEQ ID NO: 769 and the VL comprises the sequence of SEQ ID NO: 770. In some embodiments, the VH comprises the sequence of SEQ ID NO: 771 and the VL comprises the sequence of SEQ ID NO: 772. In some embodiments, the VH comprises the sequence of SEQ ID NO: 773 and the VL comprises the sequence of SEQ ID NO: 774. In some embodiments, the VH comprises the sequence of SEQ ID NO: 775 and the VL comprises the sequence of SEQ ID NO: 776. In some embodiments, the VH comprises the sequence of SEQ ID NO: 777 and the VL comprises the sequence of SEQ ID NO: 778. In some embodiments, the VH comprises the sequence of SEQ ID NO: 779 and the VL comprises the sequence of SEQ ID NO: 780. In some embodiments, the VH comprises the sequence of SEQ ID NO: 781 and the VL comprises the sequence of SEQ ID NO: 782. In some embodiments, the VH comprises the sequence of SEQ ID NO: 783 and the VL comprises the sequence of SEQ ID NO: 784. In some embodiments, the VH comprises the sequence of SEQ ID NO: 785 and the VL comprises the sequence of SEQ ID NO: 786. In some embodiments, the VH comprises the sequence of SEQ ID NO: 787 and the VL comprises the sequence of SEQ ID NO: 788. In some embodiments, the VH comprises the sequence of SEQ ID NO: 789 and the VL comprises the sequence of SEQ ID NO: 790. In some embodiments, the VH comprises the sequence of SEQ ID NO: 791 and the VL comprises the sequence of SEQ ID NO: 792. In some embodiments, the VH comprises the sequence of SEQ ID NO: 793 and the VL comprises the sequence of SEQ ID NO: 794. In some embodiments, the VH comprises the sequence of SEQ ID NO: 795 and the VL comprises the sequence of SEQ ID NO: 796. In some embodiments, the VH comprises the sequence of SEQ ID NO: 797 and the VL comprises the sequence of SEQ ID NO: 798. In some embodiments, the VH comprises the sequence of SEQ ID NO: 799 and the VL comprises the sequence of SEQ ID NO: 800. In some embodiments, the VH comprises the sequence of SEQ ID NO: 841 and the VL comprises the sequence of SEQ ID NO: 842. In some embodiments, the VH comprises the sequence of SEQ ID NO: 843 and the VL comprises the sequence of SEQ ID NO: 844. In some embodiments, the VH comprises the sequence of SEQ ID NO: 845 and the VL comprises the sequence of SEQ ID NO: 846. In some embodiments, the VH comprises the sequence of SEQ ID NO: 847 and the VL comprises the sequence of SEQ ID NO: 848. In some embodiments, the VH comprises the sequence of SEQ ID NO: 849 and the VL comprises the sequence of SEQ ID NO: 850. In some embodiments, the VH comprises the sequence of SEQ ID NO: 851 and the VL comprises the sequence of SEQ ID NO: 852. In some embodiments, the VH comprises the sequence of SEQ ID NO: 853 and the VL comprises the sequence of SEQ ID NO: 854. In some embodiments, the VH comprises the sequence of SEQ ID NO: 855 and the VL comprises the sequence of SEQ ID NO: 856. In some embodiments, the VH comprises the sequence of SEQ ID NO: 857 and the VL comprises the sequence of SEQ ID NO: 858. In some embodiments, the VH comprises the sequence of SEQ ID NO: 859 and the VL comprises the sequence of SEQ ID NO: 860. In some embodiments, the VH comprises the sequence of SEQ ID NO: 861 and the VL comprises the sequence of SEQ ID NO: 862. In some embodiments, the VH comprises the sequence of SEQ ID NO: 863 and the VL comprises the sequence of SEQ ID NO: 864. In some embodiments, the VH comprises the sequence of SEQ ID NO: 865 and the VL comprises the sequence of SEQ ID NO: 866. In some embodiments, the VH comprises the sequence of SEQ ID NO: 867 and the VL comprises the sequence of SEQ ID NO: 868. In some embodiments, the VH comprises the sequence of SEQ ID NO: 869 and the VL comprises the sequence of SEQ ID NO: 870. In some embodiments, the VH comprises the sequence of SEQ ID NO: 871 and the VL comprises the sequence of SEQ ID NO: 872. In some embodiments, the VH comprises the sequence of SEQ ID NO: 873 and the VL comprises the sequence of SEQ ID NO: 874. In some embodiments, the VH comprises the sequence of SEQ ID NO: 875 and the VL comprises the sequence of SEQ ID NO: 876. In some embodiments, the VH comprises the sequence of SEQ ID NO: 877 and the VL comprises the sequence of SEQ ID NO: 878. In some embodiments, the VH comprises the sequence of SEQ ID NO: 879 and the VL comprises the sequence of SEQ ID NO: 880. In some embodiments, the VH comprises the sequence of SEQ ID NO: 881 and the VL comprises the sequence of SEQ ID NO: 882. In some embodiments, the VH comprises the sequence of SEQ ID NO: 883 and the VL comprises the sequence of SEQ ID NO: 884. In some embodiments, the VH comprises the sequence of SEQ ID NO: 885 and the VL comprises the sequence of SEQ ID NO: 886. In some embodiments, the VH comprises the sequence of SEQ ID NO: 887 and the VL comprises the sequence of SEQ ID NO: 888. In some embodiments, the VH comprises the sequence of SEQ ID NO: 889 and the VL comprises the sequence of SEQ ID NO: 890. In some embodiments, the VH comprises the sequence of SEQ ID NO: 891 and the VL comprises the sequence of SEQ ID NO: 892. In some embodiments, the VH comprises the sequence of SEQ ID NO: 893 and the VL comprises the sequence of SEQ ID NO: 894. In some embodiments, the VH comprises the sequence of SEQ ID NO: 895 and the VL comprises the sequence of SEQ ID NO: 896. In some embodiments, the VH comprises the sequence of SEQ ID NO: 897 and the VL comprises the sequence of SEQ ID NO: 898. In some embodiments, the VH comprises the sequence of SEQ ID NO: 899 and the VL comprises the sequence of SEQ ID NO: 900. In some embodiments, the VH comprises the sequence of SEQ ID NO: 901 and the VL comprises the sequence of SEQ ID NO: 902. In some embodiments, the VH comprises the sequence of SEQ ID NO: 903 and the VL comprises the sequence of SEQ ID NO: 904. In some embodiments, the VH comprises the sequence of SEQ ID NO: 905 and the VL comprises the sequence of SEQ ID NO: 906. In some embodiments, the VH comprises the sequence of SEQ ID NO: 907 and the VL comprises the sequence of SEQ ID NO: 908. In some embodiments, the VH comprises the sequence of SEQ ID NO: 909 and the VL comprises the sequence of SEQ ID NO: 910. In some embodiments, the VH comprises the sequence of SEQ ID NO: 911 and the VL comprises the sequence of SEQ ID NO: 912. In some embodiments, the VH comprises the sequence of SEQ ID NO: 913 and the VL comprises the sequence of SEQ ID NO: 914. In some embodiments, the VH comprises the sequence of SEQ ID NO: 915 and the VL comprises the sequence of SEQ ID NO: 916. In some embodiments, the VH comprises the sequence of SEQ ID NO: 917 and the VL comprises the sequence of SEQ ID NO: 918. In some embodiments, the VH comprises the sequence of SEQ ID NO: 919 and the VL comprises the sequence of SEQ ID NO: 920. In some embodiments, the VH comprises the sequence of SEQ ID NO: 921 and the VL comprises the sequence of SEQ ID NO: 922. In some embodiments, the VH comprises the sequence of SEQ ID NO: 923 and the VL comprises the sequence of SEQ ID NO: 924. In some embodiments, the VH comprises the sequence of SEQ ID NO: 925 and the VL comprises the sequence of SEQ ID NO: 926. In some embodiments, the VH comprises the sequence of SEQ ID NO: 927 and the VL comprises the sequence of SEQ ID NO: 928. In some embodiments, the VH comprises the sequence of SEQ ID NO: 929 and the VL comprises the sequence of SEQ ID NO: 930. In some embodiments, the VH comprises the sequence of SEQ ID NO: 931 and the VL comprises the sequence of SEQ ID NO: 932. In some embodiments, the VH comprises the sequence of SEQ ID NO: 933 and the VL comprises the sequence of SEQ ID NO: 934. In some embodiments, the VH comprises the sequence of SEQ ID NO: 935 and the VL comprises the sequence of SEQ ID NO: 936. In some embodiments, the VH comprises the sequence of SEQ ID NO: 937 and the VL comprises the sequence of SEQ ID NO: 938. In some embodiments, the VH comprises the sequence of SEQ ID NO: 939 and the VL comprises the sequence of SEQ ID NO: 940. In some embodiments, the VH comprises the sequence of SEQ ID NO: 941 and the VL comprises the sequence of SEQ ID NO: 942. In some embodiments, the VH comprises the sequence of SEQ ID NO: 943 and the VL comprises the sequence of SEQ ID NO: 944. In some embodiments, the VH comprises the sequence of SEQ ID NO: 945 and the VL comprises the sequence of SEQ ID NO: 946. In some embodiments, the VH comprises the sequence of SEQ ID NO: 947 and the VL comprises the sequence of SEQ ID NO: 948. In some embodiments, the VH comprises the sequence of SEQ ID NO: 949 and the VL comprises the sequence of SEQ ID NO: 950. In some embodiments, the VH comprises the sequence of SEQ ID NO: 951 and the VL comprises the sequence of SEQ ID NO: 952. In some embodiments, the VH comprises the sequence of SEQ ID NO: 953 and the VL comprises the sequence of SEQ ID NO: 954. In some embodiments, the VH comprises the sequence of SEQ ID NO: 955 and the VL comprises the sequence of SEQ ID NO: 956. In some embodiments, the VH comprises the sequence of SEQ ID NO: 957 and the VL comprises the sequence of SEQ ID NO: 958. In some embodiments, the VH comprises the sequence of SEQ ID NO: 959 and the VL comprises the sequence of SEQ ID NO: 960. In some embodiments, the VH comprises the sequence of SEQ ID NO: 961 and the VL comprises the sequence of SEQ ID NO: 962. In some embodiments, the VH comprises the sequence of SEQ ID NO: 963 and the VL comprises the sequence of SEQ ID NO: 964. In some embodiments, the VH comprises the sequence of SEQ ID NO: 965 and the VL comprises the sequence of SEQ ID NO: 966. In some embodiments, the VH comprises the sequence of SEQ ID NO: 967 and the VL comprises the sequence of SEQ ID NO: 968. In some embodiments, the VH comprises the sequence of SEQ ID NO: 969 and the VL comprises the sequence of SEQ ID NO: 970. In some embodiments, the VH comprises the sequence of SEQ ID NO: 971 and the VL comprises the sequence of SEQ ID NO: 972. In some embodiments, the VH comprises the sequence of SEQ ID NO: 973 and the VL comprises the sequence of SEQ ID NO: 974. In some embodiments, the VH comprises the sequence of SEQ ID NO: 975 and the VL comprises the sequence of SEQ ID NO: 976. In some embodiments, the VH comprises the sequence of SEQ ID NO: 977 and the VL comprises the sequence of SEQ ID NO: 978. In some embodiments, the VH comprises the sequence of SEQ ID NO: 979 and the VL comprises the sequence of SEQ ID NO: 980, 981, 982, 983, or 984. In some embodiments, the VH comprises the sequence of SEQ ID NO: 985 and the VL comprises the sequence of SEQ ID NO: 986. In some embodiments, the VH comprises the sequence of SEQ ID NO: 987, 1687, or 1688, and the VL comprises the sequence of SEQ ID NO: 988, 989, 990, 991, or 992. In some embodiments, the VH comprises the sequence of SEQ ID NO: 993 and the VL comprises the sequence of SEQ ID NO: 994. In some embodiments, the VH comprises the sequence of SEQ ID NO: 995 and the VL comprises the sequence of SEQ ID NO: 996. In some embodiments, the VH comprises the sequence of SEQ ID NO: 997 and the VL comprises the sequence of SEQ ID NO: 998. In some embodiments, the VH comprises the sequence of SEQ ID NO: 999 and the VL comprises the sequence of SEQ ID NO: 1000, 1001, 1002, 1003, or 1004. In some embodiments, the VH comprises the sequence of SEQ ID NO: 1005 and the VL comprises the sequence of SEQ ID NO: 1006. In some embodiments, the VH comprises the sequence of SEQ ID NO: 1007 and the VL comprises the sequence of SEQ ID NO: 1008, 1009, 1010. In some embodiments, the VH comprises the sequence of SEQ ID NO: 1011 and the VL comprises the sequence of SEQ ID NO: 1012. In some embodiments, the VH comprises the sequence of SEQ ID NO: 1013 and the VL comprises the sequence of SEQ ID NO: 1014. In some embodiments, the VH comprises the sequence of SEQ ID NO: 1015 and the VL comprises the sequence of SEQ ID NO: 1016. In some embodiments, the VH comprises the sequence of SEQ ID NO: 1017 and the VL comprises the sequence of SEQ ID NO: 1018. In some embodiments, the VH comprises the sequence of SEQ ID NO: 1019 and the VL comprises the sequence of SEQ ID NO: 1020. In some embodiments, the VH comprises the sequence of SEQ ID NO: 1021 and the VL comprises the sequence of SEQ ID NO: 1022. In some embodiments, the VH comprises the sequence of SEQ ID NO: 1023 and the VL comprises the sequence of SEQ ID NO: 1024. In some embodiments, the VH comprises the sequence of SEQ ID NO: 1025 and the VL comprises the sequence of SEQ ID NO: 1026. In some embodiments, the VH comprises the sequence of SEQ ID NO: 1027 and the VL comprises the sequence of SEQ ID NO: 1028. In some embodiments, the VH comprises the sequence of SEQ ID NO: 1029 and the VL comprises the sequence of SEQ ID NO: 1030. In some embodiments, the VH comprises the sequence of SEQ ID NO: 1031 and the VL comprises the sequence of SEQ ID NO: 1032. In some embodiments, the VH comprises the sequence of SEQ ID NO: 1033 and the VL comprises the sequence of SEQ ID NO: 1034. In some embodiments, the VH comprises the sequence of SEQ ID NO: 1035 and the VL comprises the sequence of SEQ ID NO: 1036. In some embodiments, the VH comprises the sequence of SEQ ID NO: 1037 and the VL comprises the sequence of SEQ ID NO: 1038. In some embodiments, the VH comprises the sequence of SEQ ID NO: 1039 and the VL comprises the sequence of SEQ ID NO: 1040. In some embodiments, the VH comprises the sequence of SEQ ID NO: 1041 and the VL comprises the sequence of SEQ ID NO: 1042. In some embodiments, the VH comprises the sequence of SEQ ID NO: 1043 and the VL comprises the sequence of SEQ ID NO: 1044, 1689, or 1690. In some embodiments, the VH comprises the sequence of SEQ ID NO: 1045 and the VL comprises the sequence of SEQ ID NO: 1046. In some embodiments, the VH comprises the sequence of SEQ ID NO: 1047 and the VL comprises the sequence of SEQ ID NO: 1048. In some embodiments, the VH comprises the sequence of SEQ ID NO: 1049 and the VL comprises the sequence of SEQ ID NO: 1050. In some embodiments, the VH comprises the sequence of SEQ ID NO: 1051 and the VL comprises the sequence of SEQ ID NO: 1052. In some embodiments, the VH comprises the sequence of SEQ ID NO: 1053 and the VL comprises the sequence of SEQ ID NO: 1054. In some embodiments, the VH comprises the sequence of SEQ ID NO: 1055 and the VL comprises the sequence of SEQ ID NO: 1056.

In some embodiments, the antibody or antigen-binding fragment specifically binds to human LILRB1 and/or LILRB2. In some embodiments, the antibody or antigen-binding fragment is a humanized antibody or antigen-binding fragment thereof. In some embodiments, the antibody or antigen-binding fragment is a single-chain variable fragment (scFV) or a multi-specific antibody (e.g., a bispecific antibody).

In one aspect, the disclosure is related to an antibody-drug conjugate comprising the antibody or antigen-binding fragment thereof as described herein covalently bound to a therapeutic agent. In some embodiments, the therapeutic agent is a cytotoxic or cytostatic agent.

In one aspect, the disclosure is related to a method of treating a subject having cancer, the method comprising administering a therapeutically effective amount of a composition comprising the antibody or antigen-binding fragment thereof, or the antibody-drug conjugate as described herein, to the subject. In some embodiments, the subject has a solid tumor or hematological cancer. In some embodiments, the cancer is glioma, thyroid cancer, lung cancer, colorectal cancer, head and neck cancer, stomach cancer, liver cancer, pancreatic cancer, renal cancer, urothelial cancer, prostate cancer, testis cancer, breast cancer, cervical cancer, endomentrial cancer, ovarian cancer, or melanoma.

In one aspect, the disclosure is related to a method of decreasing the rate of tumor growth, the method comprising contacting a tumor cell with an effective amount of a composition comprising an antibody or antigen-binding fragment thereof, or the antibody-drug conjugate as described herein.

In one aspect, the disclosure is related to a method of killing a tumor cell, the method comprising contacting a tumor cell with an effective amount of a composition comprising the antibody or antigen-binding fragment thereof, or the antibody-drug conjugate as described herein.

In one aspect, the disclosure is related to a pharmaceutical composition comprising the antibody or antigen-binding fragment thereof, and a pharmaceutically acceptable carrier. In one aspect, the disclosure is related to a pharmaceutical composition comprising the antibody drug conjugate as described herein, and a pharmaceutically acceptable carrier.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′)₂, and Fv fragments.

As used herein, the term “human antibody” refers to an antibody that is encoded by an endogenous nucleic acid (e.g., rearranged human immunoglobulin heavy or light chain locus) present in a human. In some embodiments, a human antibody is collected from a human or produced in a human cell culture (e.g., human hybridoma cells). In some embodiments, a human antibody is produced in a non-human cell (e.g., a mouse or hamster cell line). In some embodiments, a human antibody is produced in a bacterial or yeast cell. In some embodiments, a human antibody is produced in a transgenic non-human animal (e.g., a bovine) containing an unrearranged or rearranged human immunoglobulin locus (e.g., heavy or light chain human immunoglobulin locus).

As used herein, the term “chimeric antibody” refers to an antibody that contains a sequence present in at least two different antibodies (e.g., antibodies from two different mammalian species such as a human and a mouse antibody). A non-limiting example of a chimeric antibody is an antibody containing the variable domain sequences (e.g., all or part of a light chain and/or heavy chain variable domain sequence) of a non-human (e.g., mouse) antibody and the constant domains of a human antibody. Additional examples of chimeric antibodies are described herein and are known in the art.

As used herein, the term “humanized antibody” refers to a non-human antibody which contains minimal sequence derived from a non-human (e.g., mouse) immunoglobulin and contains sequences derived from a human immunoglobulin. In non-limiting examples, humanized antibodies are human antibodies (recipient antibody) in which hypervariable (e.g., CDR) region residues of the recipient antibody are replaced by hypervariable (e.g., CDR) region residues from a non-human antibody (e.g., a donor antibody), e.g., a mouse, rat, or rabbit antibody, having the desired specificity, affinity, and capacity. In some embodiments, the Fv framework residues of the human immunoglobulin are replaced by corresponding non-human (e.g., mouse) immunoglobulin residues. In some embodiments, humanized antibodies may contain residues which are not found in the recipient antibody or in the donor antibody. These modifications can be made to further refine antibody performance. In some embodiments, the humanized antibody contains substantially all of at least one, and typically two, variable domains, in which all or substantially all of the hypervariable loops (CDRs) correspond to those of a non-human (e.g., mouse) immunoglobulin and all or substantially all of the framework regions are those of a human immunoglobulin. The humanized antibody can also contain at least a portion of an immunoglobulin constant region (Fc), typically, that of a human immunoglobulin.

Humanized antibodies can be produced using molecular biology methods known in the art. Non-limiting examples of methods for generating humanized antibodies are described herein.

As used herein, the term “single-chain antibody” refers to a single polypeptide that contains at least two immunoglobulin variable domains (e.g., a variable domain of a mammalian immunoglobulin heavy chain or light chain) that is capable of specifically binding to an antigen. Non-limiting examples of single-chain antibodies are described herein.

As used herein, the term “multimeric antibody” refers to an antibody that contains four or more (e.g., six, eight, or ten) immunoglobulin variable domains. In some embodiments, the multimeric antibody is able to crosslink one target molecule (e.g., LILRB1, LILRB2, or LILRB3) to at least one second target molecule (e.g., LILRB1, LILRB2, or LILRB3) on the surface of a mammalian cell (e.g., a human T-cell).

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody, the phrases “specifically binding” and “specifically binds” mean that the antibody interacts with its target molecule (e.g., LILRB1 and/or LILRB2) preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to a LILRB1 and/or LILRB2 molecule may be referred to as a LILRB1 and/or LILRB2-specific antibody or an anti-LILRB1/2 antibody.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1F are graphs showing 19 out of 46 antibodies bind to both LILRB1 and LILRB2, and block the LILRB1/HLA-G and LILRB2/HLA-G interaction. CHO-LILRB1 or CHO-LILRB2 cells were incubated with titrated anti-LILRB1/2 antibodies and then Violet™ 421-labeled anti-human Fc antibody and APC-labeled tetramer of HLA-G/B2M/peptide trimer or tetramer of HLA-G monomer (HLA-G free chain). The antibody binding signals of Violet™ 421 (gMFI) to LILRB1 or LILRB2, and the antibody blocking signals of APC HLA-G were measured by flow cytometry. 3C1 and 1E1(QE) are reference antibodies. IgG1 is an isotype control (IgG). Each one of 48 figures contains a panel which includes 4 plots with vertical Y-axis of MFI and horizontal X-axis of the titration of antibody (5, 1, 0.1 and 0 μg/mL). HLA-G binding signals (MFI) were normalized to the signal without antibody blocking (0 μg/mL). Up-left plot shows HLA-G binding to CHO cells expressed RB1; up-right plot shows HLA-G binding to CHO cells expressed RB2; low-left plot shows binding of anti-RB1/2 to CHO cells expressed RB1, low-right shows binding of anti-RB1/2 to CHO cells expressed RB2.

FIG. 1G is a schematic diagram to describe the information on FIGS. 1A-IF, and representative plot labels and legends for each figure of each sample in FIGS. 1A-1F.

FIG. 2A shows the anti-LILRB1/2 antibodies binding to LILRB1 in CHO-LILRB1 cells. CHO-LILRB1 cells were incubated with titrated anti-LILRB1/2 antibodies, and then Violet™ 421-labeled anti-human Fc antibody and APC-labeled tetrameric HLA-G trimer. The antibody binding signals of Violet™ 421 (gMFI) to LILRB1 were measured by flow cytometry.

FIG. 2B shows the anti-LILRB1/2 antibodies blocking the LILRB1/HLA-G interaction. CHO-LILRB1 cells were incubated with titrated anti-LILRB1/2 antibodies, and then Violet™ 421-labeled anti-human Fc antibody and APC-labeled tetrameric HLA-G trimer. The antibody blocking signals of APC (gMFI) to HLA-G were measured by flow cytometry.

FIG. 2C shows the blocking efficiencies (IC50) and binding EC50 values.

FIG. 3A shows humanized and optimized anti-LILRB1/2 antibodies binding to LILRB1 in Jurkat-LILRB1 cells. Jurkat-LILRB1 cells were incubated with titrated anti-LILRB1/2 antibodies, and then Violet™ 421-labeled anti-human Fc antibody and APC-labeled tetrameric HLA-G trimer (recombinant HLA-G/B2M/peptide). The antibody binding signals of Violet™ 421 (gMFI) to LILRB1 were measured by flow cytometry.

FIG. 3B shows humanized and optimized anti-LILRB1/2 antibodies blocking the LILRB1/HLA-G interaction. Jurkat-LILRB1 cells were incubated with titrated anti-LILRB1/2 antibodies, and then Violet™ 421-labeled anti-human Fc antibody and APC-labeled tetrameric HLA-G trimer. The antibody blocking signals of APC (gMFI) to HLA-G were measured by flow cytometry.

FIG. 3C shows humanized and optimized anti-LILRB1/2 antibodies binding to LILRB2 in Jurkat-LILRB2 cells. Jurkat-LILRB2 cells were incubated with titrated anti-LILRB1/2 antibodies, and then Violet™ 421-labeled anti-human Fc antibody and APC-labeled tetrameric HLA-G monomer (free-chain HLA-G). The antibody binding signals of Violet™ 421 (gMFI) to LILRB2 were measured by flow cytometry.

FIG. 3D shows humanized and optimized anti-LILRB1/2 antibodies blocking the LILRB2/HLA-G interaction. Jurkat-LILRB2 cells were incubated with titrated anti-LILRB1/2 antibodies, and then Violet™ 421-labeled anti-human Fc antibody and APC-labeled tetrameric HLA-G monomer. The antibody blocking signals of APC (gMFI) to HLA-G were measured by flow cytometry.

FIG. 3E shows the binding EC50 values to LILRB1/B2 and the inhibition efficiencies (IC50) of blocking HLA-G binding to LILRB1/B2.

FIG. 4A shows the binding affinities of anti-LILRB1/2 antibodies against LILRB1 or LILRB2 measured by Carterra© LSA.

FIGS. 4B-4C show the binding affinities of D2M002-002 to LILRB1 and LILRB2 measured by Gator, respectively.

FIG. 5A shows the binding kinetic plots and fits of hit D2M002-002 and its humanized variant D2M002-103 against LILRB1 or LILRB2 measured by Carterra® LSA.

FIG. 5B shows the binding kinetic plots and fits of hit D2M002-026 and its humanized and optimized variant D2M002-126 against LILRB1 or LILRB2 measured by Carterra® LSA.

FIG. 5C shows the binding affinities of hits and selected humanized and optimized anti-LILRB1/2 against LILRB1 and LILRB2 measured by by Carterra® LSA, respectively.

FIG. 6 shows the binding affinities of hits and selected humanized and optimized anti-LILRB1/2 to human LILRB1, LILRB2 and cynomolgus monkey LILRB2 measured by Gator, respectively.

FIGS. 7A-7B show the anti-LILRB1/2 antibodies bound to LILRB1 and LILRB2 in CHO cell lines, respectively. CHO-LILRB1 or CHO-LILRB2 cells were incubated with titrated anti-LILRB1/2 antibodies and then Violet™ 421-labeled anti-human Fc antibody. Anti-LILRB1 antibody 3C1 and anti-LILRB2 antibody 1E1(QE) are reference antibodies. IgG1 is an isotype control (IgG). The antibody binding signals of Violet™ 421 (gMFI) to LILRB1 and LILRB2 were measured by flow cytometry.

FIG. 7C is a table showing the maximum MFI and calculated EC50 of anti-LILRB1/2 antibodies against LILRB1 and LILRB2. 3C1 and 1E1(QE) are reference antibodies. IgG1 is an isotype control (IgG).

FIG. 8A show Jurkat T cell activation from co-cultures of C1R-OKT3-HLAG with Jurkat-LILRB1, in the presence of anti-LILRB1/2 antibodies. The anti-LILRB1/2 antibodies reversed the activation of Jurkat cells suppressed by the HLA-G. 3C1 is a reference antibody. IgG1 is an isotype control (IgG). Co-culture of C1R-OKT3 is the control without HLA-G inhibitory signal. The luciferase signal was measured based on the luciferase-luciferin signal (RLU) driven by NFAT under membrane anti-CD3 stimulation.

FIG. 8B show Jurkat T cell activation from co-cultures of C1R-OKT3-HLAG with Jurkat-LILRB2, in the presence of anti-LILRB1/2 antibodies. The anti-LILRB1/2 antibodies reversed the activation of Jurkat cells suppressed by the HLA-G. 1E1(QE) is a reference antibody. IgG1 is an isotype control (IgG). Coculture of C1R-OKT3 is the control without HLA-G inhibitory signal. The luciferase signal was measured based on the luciferase-luciferin signal (RLU) driven by NFAT under membrane anti-CD3 stimulation.

FIG. 9A shows Jurkat T cell activation from co-cultures of C1R-OKT3-HLAG with Jurkat-LILRB1, in the presence of titrated anti-LILRB1/2 antibodies. The anti-LILRB1/2 antibodies restored the activation of Jurkat cells suppressed by the HLA-G. IgG1 is an isotype control (IgG). The luciferase signal was measured based on the luciferase-luciferin signal (RLU) driven by NFAT under membrane anti-CD3 stimulation.

FIG. 9B shows Jurkat T cell activation from co-cultures of C1R-OKT3-HLAG with Jurkat-LILRB2, in the presence of titrated anti-LILRB1/2 antibodies. The anti-LILRB1/2 antibodies reversed the activation of Jurkat cells suppressed by the HLA-G. IgG1 is an isotype control (IgG). The luciferase signal was measured based on the luciferase-luciferin signal (RLU) driven by NFAT under membrane anti-CD3 stimulation.

FIG. 9C summarizes the EC50 values of anti-LILRB1/2 antibodies to restore the activation of Jurkat cells suppressed by the HLA-G.

FIG. 10A shows TNFα concentration in anti-LILRB1/2 antibody-treated PBMC culture supernatants after LPS stimulation. PBMCs were stimulated by 50 ng/mL LPS in the presence of anti-LILRB1/2 antibodies (#2, #8, #11, #22, #26, or #27) or reference antibodies (3C1 or 1E1(QE)). IgG1 is an isotype control (IgG). TNFα concentrations were quantified by ELISA.

FIG. 10B shows IL10 concentration in anti-LILRB1/2 antibody-treated PBMC culture supernatants after LPS stimulation. PBMCs were stimulated by 50 ng/mL LPS in the presence of anti-LILRB1/2 antibodies (#2, #8, #11, #22, #26, or #27) or reference antibodies (3C1 or 1E1(QE)). IgG1 is an isotype control (IgG). IL10 concentrations were quantified by ELISA.

FIG. 11A shows the measurement results of TNFα concentrations in titrated anti-LILRB1/2 antibody-treated PBMC culture supernatants after LPS stimulation. PBMCs were stimulated by 50 ng/mL LPS in the presence of titrated anti-LILRB1/2 antibody (D2M002-126) or reference antibody (Hz73D1.v1). IgG1 is an isotype control (IgG1-LALA). TNFα concentrations were quantified by ELISA. Plots were assembled from data of 3 healthy donors.

FIG. 11B shows the measurement results of IL10 concentrations in titrated anti-LILRB1/2 antibody-treated PBMC culture supernatants after LPS stimulation. PBMCs were stimulated by 50 ng/mL LPS in the presence of anti-LILRB1/2 antibody (D2M002-126) or reference antibody (Hz73D1.v1). IgG1 is an isotype control (IgG1-LALA). IL10 concentrations were quantified by ELISA. Plots were assembled from data of 3 healthy donors.

FIGS. 12A-12F show the expression profiles of phenotypic markers of CD14, CD11B, CD86, CD163, CD206 and PD-L1, respectively, as quantified by flow cytometry. Human monocytes were differentiated into macrophages with M-SCF in the presence of anti-LILRB1/2 antibodies (#2, #8, #11, #22, #26, or #27), reference antibodies (3C1 or 1E1(QE)), or IgG1 isotype control (IgG). Each dot represents one donor. P-values were calculated by t-test. * P<0.05, ** P<0.01, *** P<0.001, **** P<0.0001, n.s. not significant.

FIGS. 13A-13D show that humanized and optimized anti-LILRB1/2 promote inflammatory expression profiles of phenotypic markers of CD14, CD11B, CD206, and PD-L1, respectively, as quantified by flow cytometry. Human monocytes were differentiated into macrophages with M-SCF in the presence of anti-LILRB1/2 antibodies as indicated or IgG1-LALA-PA isotype control or PBS. Each dot represents one donor. P-values were calculated by t-test. * P<0.05, ** P<0.01, *** P<0.001, **** P<0.0001, n.s. not significant.

FIGS. 14A-14C show the expression profiles of PD-L1, TNFα, and IL10, respectively. Differentiated macrophages treated with anti-LILRB1/2 antibodies (#2, #8, #11, #22, #26, or #27), reference antibodies (3C1 or 1E1(QE)), or IgG1 isotype control (IgG) were stimulated with LPS (100 ng/mL). Expression of phenotypic marker PD-L1 was quantified by flow cytometry, and concentrations of cytokines (TNFα and IL10) in the culture supernatants were quantified by ELISA. Each dot represents one donor. P-values were calculated by t-test. * P<0.05, ** P<0.01, *** P<0.001, **** P<0.0001, n.s. not significant.

FIGS. 15A-15B show that humanized and optimized anti-LILRB1/2 induce functional phenotype, represented by the expression profiles of TNFα and IL10, respectively. Differentiated macrophages treated with anti-LILRB1/2 antibodies or IgG1_LALA-PA isotype control or PBS were stimulated with LPS (100 ng/mL). Concentrations of cytokines (TNFα and IL10) in the culture supernatants were quantified by ELISA. Each dot represents one donor. P-values were calculated by t-test. * P<0.05, ** P<0.01, *** P<0.001, **** P<0.0001, n.s. not significant.

FIGS. 16A-16F show the expression profiles of phenotypic markers of CD14, CD11B, CD86, CD163, CD206 and PD-L1, respectively, as quantified by flow cytometry. Human monocytes were co-cultured with human breast cancer cells MDA-MB-231 to induce TAMs in the presence of anti-LILRB1/2 antibodies (#2, #8, #11, #22, #26, or #27), reference antibodies (3C1 or 1E1(QE)), or IgG1 isotype control (IgG). Each dot represents one donor. P-values were calculated by t-test. * P<0.05, ** P<0.01, *** P<0.001, **** P<0.0001, n.s. not significant.

FIG. 16G shows proportion of tumor cells in the co-culture. Human monocytes were co-cultured with human breast cancer cells MDA-MB-231 to induce TAMs in the presence of anti-LILRB1/2 antibodies (#2, #8, #11, #22, #26, or #27), reference antibodies (3C1 or 1E1(QE)), or IgG1 isotype control (IgG). The anti-LILRB1/2 antibodies and 1E1(QE) inhibited the growth of tumor cells. Each dot represents one donor. P-values were calculated by t-test. * P<0.05, ** P<0.01, *** P<0.001, **** P<0.0001, n.s. not significant.

FIGS. 17A-17D show that humanized and optimized anti-LILRB1/2 promote inflammatory expression profiles of phenotypic markers of CD14, CD86, CD206 and PD-L1, respectively, as quantified by flow cytometry. Human monocytes were co-cultured with human breast cancer cells MDA-MB-231 to induce TAMs in the presence of anti-LILRB1/2 antibodies as indicated, or IgG1LALA-PA isotype control or PBS. Each dot represents one donor. P-values were calculated by t-test. * P<0.05, ** P<0.01, *** P<0.001, **** P<0.0001, n.s. not significant.

FIG. 17E shows that humanized and optimized anti-LILRB1/2 inhibited tumor growth in vitro. Human monocytes were co-cultured with human breast cancer cells MDA-MB-231 to induce TAMs in the presence of anti-LILRB1/2 antibodies or IgG1-LALA-PA isotype control or PBS. The proportion of tumor cells in the co-culture was measured using FACS. Each dot represents one donor. P-values were calculated by t-test. * P<0.05, ** P<0.01, *** P<0.001, **** P<0.0001, n.s. not significant.

FIGS. 18A-18C show the expression profiles of PD-L1, TNFα, and IL10, respectively. TAMs treated with anti-LILRB1/2 antibodies (#2, #8, #11, #22, #26, or #27), reference antibodies (3C1 or 1E1(QE)), or IgG1 isotype control (IgG) were stimulated with LPS (100 ng/mL). Expression of phenotypic marker PD-L1 was quantified by flow cytometry, and concentrations of cytokines (TNFα and IL10) in the culture supernatants were quantified by ELISA. Each dot represents one donor. P-values were calculated by t-test. * P<0.05, ** P<0.01, *** P<0.001, **** P<0.0001, n.s. not significant.

FIGS. 19A-19B show the expression profiles of TNFα and IL10, respectively. TAMs treated with anti-LILRB1/2 antibodies or IgG1_LALA-PA isotype control or PBS were stimulated with LPS (100 ng/mL). Concentrations of cytokines (TNFα and IL10) in the culture supernatants were quantified by ELISA. Each dot represents one donor. P-values were calculated by t-test. * P<0.05, ** P<0.01, *** P<0.001, **** P<0.0001, n.s. not significant.

FIG. 20 shows IFN-γ concentrations in MLR culture supernatants by ELISA. DCs were generated from monocytes with GM-CSF/IL4, and matured by LPS. Mixed lymphocyte reaction (MLR) was performed by co-culturing mature DCs and allotype T cells (negatively isolated from human PBMCs) in the presence of differentiated hMDM by anti-LILRB1/2 antibodies ((#2, #8, #11, #22, #26, or #27). 3C1 and 1E1(QE) are reference antibodies. IgG1 is an isotype control (IgG). T cell activation was measured based on the expression of IFN-γ in MLR supernatants. “w/o Mac” stands for without macrophages. Each dot represents one combination of MLR donor pair and hMDM. P-values were calculated by t-test. * P<0.05, ** P<0.01, *** P<0.001, P<0.0001, n.s. not significant.

FIG. 21 shows the proportion of GFP+ hMDMs in total hMDMs (indicating phogocytosis) as measured by flow cytometry. C1R-HLAG-GFP cells were equally co-cultured with the hMDMs in the presence of anti-LILRB1/2 antibodies (#2, #8, #11, #26, or #27). 3C1 and 1E1(QE) are reference antibodies. IgG1 is an isotype control (IgG). Clone #4 can only bind to LILRB1 and clone #44 can only bind to LILRB2. Each dot represents one donor. P-values were calculated by t-test. * P<0.05, ** P<0.01, *** P<0.001, **** P<0.0001, n.s. not significant.

FIG. 22 shows the proportion of GFP+ hMDMs in total hMDMs (indicating antibody-dependent cellular phagocytosis, or ADCP) as measured by flow cytometry. The tumor cells were labeled with Rituximab (anti-CD20) for ADCP, and equally co-cultured with the hMDMs in the presence of anti-LILRB1/2 antibodies (#2, #8, #11, #26, or #27). 3C1 and 1E1(QE) are reference antibodies. IgG1 is an isotype control (IgG). Clone #4 can only bind to LILRB1 and clone #44 can only bind to LILRB2. Each dot represents one donor. P-values were calculated by t-test. * P<0.05, ** P<0.01, *** P<0.001, **** P<0.0001, n.s. not significant.

FIGS. 23A-23B show the proportion of GFP+ hMDMs in total hMDMs (indicating antibody-dependent cellular phagocytosis, or ADCP) as measured by flow cytometry. The A375 tumor cells were labeled with anti-CD47 for ADCP, and equally co-cultured with the hMDMs in the presence of anti-LILRB1/2 antibodies. 3C1 and 15G8 are anti-LILRB1 reference antibodies. 1E1(QE) and J-19-H1 are anti-LILRB2 reference antibodies. Hz73D1.v1 is the anti-LILRB1/2 reference antibody. IgG1-LALA is an isotype control. P-values were calculated by t-test. * P<0.05, ** P<0.01, *** P<0.001, **** P<0.0001, n.s. not significant.

FIG. 24A shows histograms of APC signals from CHO-cynoLILRB1 or CHO-cynoLILRB2 cells after incubation with 2 μg/mL anti-LILRB1/2 antibodies followed with APC anti-hFc. 3C1 and 1E1(QE) are reference antibodies. IgG1 is an isotype control (IgG).

FIG. 24B shows the antibody binding signals to monkey LILRB2 (cynoLILRB2) as measured by flow cytometry. CHO-cynoLILRB2 cells were incubated with titrated anti-LILRB1/2 antibodies followed with APC anti-hFc. IgG1 is an isotype control (IgG).

FIG. 25A shows the binding signals of anti-LILRB1/2 antibodies to LILRB1, LILRB2, LILRB3, LILRB4, and LILRB5. 293T cells transiently expressing different LILRB proteins were incubated with anti-LILRB1/2 antibodies (5 μg/mL), followed by APC anti-human Fc. The antibody binding signals were measured by flow cytometry with geometric MFI (gMFI). 3C1 and 1E1(QE) are reference antibodies. IgG1 is an isotype control (IgG).

FIG. 25B shows the binding signals of anti-LILRB1/2 antibodies to LILRA1, LILRA2, LILRA3, LILRA4, LILRA5 and LILRA6. 293T cells transiently expressing different LILRB proteins were incubated with anti-LILRB1/2 antibodies (5 μg/mL), followed by APC anti-human Fc. The antibody binding signals were measured by flow cytometry with geometric MFI (gMFI). 3C1 and 1E1(QE) are reference antibodies. IgG1 is an isotype control (IgG).

FIG. 25C shows the antibody binding signals to LILRB3 as measured by flow cytometry. CHO-LILRB3 cells were incubated with titrated anti-LILRB1/2 antibodies followed with APC anti-hFc. IgG1 is an isotype control (IgG).

FIGS. 26A-26F show the binding characteristics of the anti-LILRB1/2 antibodies to human LILRB1, LILRB2, LILRB3, cynomolgus monkey LILRB2 (cynoLILRB2), human LILRA1 and LILRA6 in CHO cell lines, respectively. CHO-LILRB1, or CHO-LILRB2, or CHO-LILRB3, or CHO-cynoLILRB2, or CHO-LILRA1 or CHO-LILRA6 cells were incubated with titrated anti-LILRB1/2 antibodies and then Violet™ 421-labeled anti-human Fc antibody. IgG1 is an isotype control (IgG). The antibody binding signals of Violet™ 421 (gMFI) to LILRB1, LILRB2, LILRB3, cynoLILRB2, LILRA1 and LILRA6 were measured by flow cytometry.

FIG. 26G is a table summarized the maximum MFI and calculated EC50 of anti-LILRB1/2 antibodies against LILRB1, LILRB2, LILRB3, cynoLILRB2, LILRA1 and LILRA6. N.B. indicates NOT BIND.

FIGS. 27A-27F show the anti-LILRB1/2 antibody D2M002-126 blocking the interactions of LILRB2 and reported putative ligands SEMA4A, MAG, CD1C, CD1D, ANGPTL2 and ANGPTL5. Plates coated ligands were incubated with the mixture of the biotinylated LILRB2-hFc and titrated D2M002-126 or isotype control (IgG1-LALA). The binding signals of ligand and LILRB2 were detected by streptavidin HRP and substrate TMB.

FIG. 28 shows the cytotoxicity of RB1-transduced NK cells against A253 tumor cells measured by flow cytometry. The human primary NK cells were transduced with a lentiviral particle to express LILRB1 and cocultured with the A253-GFP tumor cells equally in the presence of anti-LILRB1/2 antibodies or isotype control or PBS. The proportion of dead tumor cells (GFP+DAPI+) in total tumor cells (GFP+) were quantified by flow cytometry. Hz73D1.v1 is the anti-LILRB1/2 reference antibody. IgG1-LALA is an isotype control. P-values were calculated by t-test. * P<0.05, ** P<0.01, *** P<0.001, **** P<0.0001, n.s. not significant.

FIG. 29 shows the cytotoxicity of RB1-transduced CD8+ T cells against CIR tumor cells expressing anti-CD3 scFv and HLA-G measured by flow cytometry. The human primary CD8+ T cells were activated and transduced with a lentiviral particle to express LILRB1. CD8+ T cells were cocultured equally with the C1R-GFP cells engineered to express anti-CD3 scFv and HLA-G, in the presence of anti-LILRB1/2 antibodies or isotype control. The proportion of dead tumor cells (GFP+DAPI+) in total tumor cells (GFP+) were quantified by flow cytometry. 3C1 and 15G8 are anti-LILRB1 reference antibodies. Hz73D1.v1 is the anti-LILRB1/2 reference antibody. IgG1-LALA is an isotype control.

FIG. 30A is a plot showing the cell-based binding affinity of humanized and optimized anti-LILRB1/2 antibody that had been stressed in human serum or PBS at 37° C. for 1, 2 and 3 weeks in CHO-LILRB1 cells. CHO-LILRB1 cells were incubated with titrated anti-LILRB1/2 antibody as indicated, and then Violet™ 421-labeled anti-human Fc antibody. The antibody binding signals of Violet™ 421 (gMFI) to LILRB1 were measured by flow cytometry.

FIG. 30B is a plot showing the cell-based binding affinity of humanized and optimized anti-LILRB1/2 antibody that had been stressed in human serum or PBS at 37 degrees for 1, 2 and 3 weeks in CHO-LILRB2 cells. CHO-LILRB2 cells were incubated with titrated anti-LILRB1/2 antibody as indicated, and then Violet™ 421-labeled anti-human Fc antibody. The antibody binding signals of Violet™ 421 (gMFI) to LILRB2 were measured by flow cytometry.

FIG. 31 shows the abundance of D2M002-126 in vivo plotted over time. Single dose (10 mg/kg) of D2M002-126 was dosed i.v. into C57BL/6 mice. Blood was sampled at designated timepoints. D2M002-126 was quantified by a sandwich ELISA using anti-human Fab as capture reagent and anti-human FC as detection reagent.

FIG. 32 shows the tumor volume of NCG mice that were subcutaneously co-implanted with a mixture of A375 tumor cells and human MDMs and treated with D2M002-126 and an isotype control (IgG-1-LALA-PA) twice weekly for total 3 weeks.

FIG. 33 shows the tumor volume of NCG mice that were implanted with A375 tumor cells. When tumors grow to 50-100 mm³, human MDMs were injected intratumorally, and tumor-bearing mice were treated with antibodies and an isotype control twice weekly for total 3 weeks.

FIG. 34 shows the tumor volume of NCG mice with immune system reconstituted with human PBMC, that were implanted with A375 tumor cells. Mice were grouped and treated with D2M002-126 and an isotype control (IgG1) twice weekly for total 4 weeks.

FIGS. 35A-35B show D2M002-002 variants and D2M026 variants have distinct binding epitopes. Epitope binning analyses were performed by the Gator™ biolayer interferometry platform with GatorPrime.

FIG. 35C shows the competitive binding of D2M002-126 against LILRB1. D2M002-126 does not compete with reference anti-LILRB1 antibody 15G8. 15G8 is the anti-LILRB1 reference antibody. Hz73D1.v1 is the anti-LILRB1/2 reference antibody.

FIG. 35D shows the competitive binding of D2M002-126 against LILRB2. Although D2M002-126 competes with Hz73D1.v1, D2M002-126 does not compete with anti-LILRB2 reference antibody J-19.H1 whereas 1E1(QE) and Hz73D1.v1. competes with J-19.H1. 1E1(QE) and J-19-H1 are anti-LILRB2 reference antibodies. Hz73D1.v1 is the anti-LILRB1/2 reference antibody.

FIG. 36 lists CDR sequences of anti-LILRB1/2 antibodies (D2M002-001, D2M002-002, D2M002-003, D2M002-004, D2M002-008, D2M002-011, D2M002-012, D2M002-016, D2M002-017, D2M002-018, D2M002-020, D2M002-021, D2M002-022, D2M002-023, D2M002-026, D2M002-027, D2M002-028, D2M002-029, D2M002-031, D2M002-035, D2M002-036, D2M002-040, D2M002-041, D2M002-044, and D2M002-045) as defined by IMGT, Kabat, Chothia, North, or Aho numbering scheme.

FIG. 37 lists amino acid sequences of heavy chain variable regions and light chain variable regions of anti-LILRB1/2 antibodies.

FIG. 38 lists amino acid sequences of relevant antibodies or fragments thereof.

FIG. 39 lists amino acid sequences of the heavy chain and light chain of D2M002-002 formatted in mouse IgG1.

FIG. 40 lists amino acid sequences of customized proteins.

FIG. 41 lists amino acid sequences of other proteins discussed in the disclosure.

FIG. 42 lists CDR sequences of D2M002-002 lineage humanized and optimized variants as defined by IMGT, Kabat, Chothia, or North numbering.

FIG. 43 lists CDR sequence of D2M002-026 lineage humanized and optimized variants as defined by IMGT, Kabat, Chothia, or North numbering.

FIG. 44 lists VH and VL sequences of D2M002-002 lineage humanized and optimized variants.

FIG. 45 lists VH and VL sequences of D2M002-026 lineage humanized and optimized variants.

FIG. 46 lists heavy and light chain sequences of humanized and optimized variants.

### DETAILED DESCRIPTION

The present disclosure provides examples of antibodies, antigen-binding fragment thereof, that bind to LILRB1 (Leukocyte immunoglobulin-like receptor subfamily B member 1; also known as ILT2) and/or LILRB2 (Leukocyte immunoglobulin-like receptor subfamily B member 2; also known as ILT4).

### LILRB1, LILRB2 and HLA-G

The major histocompatibility complex (MHC) plays a key role in the defense mechanism of a body induced by T cell immune responses by presenting a cancer- or virus-derived antigen peptide. Humans have MHC class I and class II genes. The classical class I genes are termed HLA-A, HLA-B and HLA-C. Minor MHC Class I genes in HLA are HLA-E, HLA-F and HLA-G.

HLA-G is a nonclassical MHC class I molecule that plays a crucial role in fetal-maternal tolerance. It is an inhibitory molecule regulating T cell activation. HLA-G via interaction of the LILRB1 (ILT2) and LILRB2 (ILT4) receptors inhibits cytotoxic T cells, natural killer (NK) cells, and B cells, induces T cell anergy, modulates myeloid cells, and promotes T regulatory cells (Tregs). Moreover, HLA-G expressed on antigen-presenting cells (APCs), such as myeloid-derived suppressor cells (MDSCs) or tolerogenic dendritic cells (DCs), promotes T cell hyporesponsiveness and induces Treg differentiation. LILRB1 is expressed on different leukemia and solid tumors. While LILRB1 prevents primary cutaneous T cell lymphoma cell death and enhances gastric tumor growth, HLA-G/LILRB1 interaction inhibits neoplastic B cell proliferation. Blocking LILRB1 on myeloma or lymphoblastic cells does not prevent NK-mediated lysis. LILRB2 is expressed in various solid tumors such as clear cell renal carcinoma (ccRCC) and stroma cells within the tumor microenvironment. LILRB2 expression correlates with poor cell differentiation and advanced metastasis. One underlying mechanism is the upregulation of vascular endothelial growth factor (VEGF)-C, a well-characterized and efficient growth factor involved in lymphangiogenesis and lymphatic metastases.

In immunocompetent mice, HLA-G-expressing tumor cells proliferate, while in immunodeficient mice, administration of HLA-G-expressing tumor cells results in widespread metastasis, suggesting that HLA-G plays a role in immune escape in tumors. Moreover, de novo expression of HLA-G is found in most human tumors analyzed, but not in surrounding healthy voluntary tissues. These findings are consistent with the involvement of the checkpoint HLA-G in tumor progression and invasiveness. Moreover, HLA-G/LILRBs affect a wider spectrum of immune cells compared to those modulated by cytotoxic T lymphocyte-associated antigen (CTLA)-4/B7 and programmed death (PD)-1/PD ligand-1; known targets of current IC blockade therapies. Therefore, the checkpoint HLA-G/LILRB interaction is promising as an additional target for cancer immunotherapy.

LILR (also known as immunoglobulin-like transcripts (ILTs) or LIR) are a family of 11 receptors that have two to four extracellular Ig-like domains, and are categorized as inhibitory or activating. Most LILR are expressed as membrane-bound receptors, except LILRA3 which is exclusively expressed in a soluble form. The inhibitory receptors (LILRB1 to B5) possess long cytoplasmic tails containing ITIMs, whilst the activating receptors (LILRA1 to A6, excluding A3) possess short cytoplasmic tails and couple with ITAM-bearing FcεRIγ. Individual LILR receptors are classified as group 1 (LILRB1, LILRB2, and LILRA1-3) or group 2 (LILRB3-5 and LILRA4-6) members, based on conservation of LILRB1 residues that recognize human leukocyte antigen (HLA) class I molecules. Expression of individual LILR has been documented for a range of immune cells including neutrophils, eosinophils, macrophage, dendritic cells, NK cells, B cells, T cells, and osteoclasts and non-immune cells such as endothelial cells and neurons. Most LILR genes additionally encode soluble forms of LILR produced by alternative splicing. LILR orthologs found in mice are called PIR; however, there are fundamental differences within human LILR. For example, PIR possess six Ig-like domains and there are only two inhibitory receptors called PIR-B and gp49b1. Human LILRB and PIR-B can modulate the functions of ITAM-bearing receptors such as FcR, B cell receptor (BCR), and T cell receptor (TCR). LILR also modulate toll-like receptor (TLR) signaling and functions. Thus, LILR can modulate a broad set of immune functions, including immune cell function, cytokine release, antibody production, and antigen presentation.

LILRB1 and LILRB2 belong to the subfamily B class of LIR receptors which contain two or four extracellular immunoglobulin domains, a transmembrane domain, and two to four cytoplasmic immunoreceptor tyrosine-based inhibitory motifs (ITIMs). The receptor is expressed on immune cells where it binds to MHC class I molecules on antigen-presenting cells and transduces a negative signal that inhibits stimulation of an immune response. It is thought to control inflammatory responses and cytotoxicity to help focus the immune response and limit autoreactivity. Multiple transcript variants encoding different isoforms have been found. Both LILRB1 and LILRB2 are expressed on a variety of human immune cells. This includes all B cells, monocytes and macrophages, dendritic cells and subsets of NK cells and T cells. The percentage of LILRB1+ NK cells is significantly higher in patients with advanced stage prostate and breast cancer than in healthy voluntary donors or patients with localized cancer. Blockade of LILRB1 signaling in immune cells was capable of activating the activity of NK cells against solid tumor and leukemia, and activating T cells or macrophages against solid tumors, using in vitro models.

Further, LILRB1 was found on the surface of macrophages, and it can bind to a portion of MHC class I on cancer cells that is widely shared across individuals. This binding inhibits the ability of macrophages to engulf and kill the cancer cells, both when growing a laboratory dish and in mice with human tumors. Thus, LILRB1 was considered as the second “don't eat me” signal found on cancer cells, after the CD47/SIRPα pathway.

As LILRB1 and LILRB2 are promising immunomodulatory targets for cancer immunotherapy, LILRB1 and/or LILRB2-specific antibodies can be designed, e.g., to block LILRB1/HLA-G and/or LILRB2/HLA-G interactions, thereby increasing immune response. A detailed description regarding LILRB1, LILRB2, and their roles for cancer immunotherapy can be found, e.g., in Carosella, E. D., et al. “HLA-G/LILRBs: A Cancer Immunotherapy Challenge.” Trends in Cancer 7.5 (2021): 389-392; Chen, H., et al. “Antagonistic anti-LILRB1 monoclonal antibody regulates antitumor functions of natural killer cells.” Journal for Immunotherapy of Cancer 8.2 (2020); Lewis M., et al. “Leukocyte immunoglobulin-like receptors (LILRs) on human neutrophils: modulators of infection and immunity.” Frontiers in immunology (2020): 857; Wang, Q., et al. “Structures of the four Ig-like domain LILRB2 and the four-domain LILRB1 and HLA-G1 complex.” Cellular & Molecular Immunology 17.9 (2020): 966-975; and Barkal, A., et al. “Engagement of MHC class I by the inhibitory receptor LILRB1 suppresses macrophages and is a target of cancer immunotherapy.” Nature Immunology 19.1 (2018): 76-84; which are incorporated herein by reference in the entirety.

The present disclosure provides several anti-LILRB1/2 antibodies, antigen-binding fragments thereof, and methods of using these antibodies and antigen-binding fragments to inhibit tumor growth and to treat cancers.

### Anti-LILRB1/2 Antibodies and Antigen-Binding Fragments

The disclosure provides antibodies and antigen-binding fragments thereof that specifically bind to LILRB1 and/or LILRB2. The antibodies and antigen-binding fragments described herein are capable of binding to LILRB1 and/or LILRB2, and can inhibit the suppressive signaling pathway thus increase immune response. The disclosure provides e.g., mouse anti-LILRB1/2 antibodies D2M002-001 (“#1”), D2M002-002 (“#2”), D2M002-003 (“#3”), D2M002-004 (“#4”), D2M002-008 (“#8”), D2M002-011 (“#11”), D2M002-012 (“#12”), D2M002-016 (“#16”), D2M002-017 (“#17”), D2M002-018 (“#18”), D2M002-020 (“#20”), D2M002-021 (“#21”), D2M002-022 (“#22”), D2M002-023 (“#23”), D2M002-026 (“#26”), D2M002-027 (“#27”), D2M002-028 (“#28”), D2M002-029 (“#29”), D2M002-031 (“#31”), D2M002-035 (“#35”), D2M002-036 (“#36”), D2M002-040 (“#40”), D2M002-041 (“#41”), D2M002-044 (“#44”), D2M002-045 (“#45”), D2M002-002-H12, D2M002-002-H26, D2M002-026-H14, D2M002-026-H25, P215_C06, P215_H02, P212_E10, P211_E01, P211_B01, P211_G04, P211_C01, P212_C04, P212_F06, P211_B02, P213_C03, P211_C02, P211_C09, P212_A03, P212_B07, P213_C05, P213_H11, P211_E07, P213_G10, P212_E03, P212_G10, P215_A01, P211_B05, P215_H01, P212_D01, P215_F02, P212_H04, P212_D04, P211_E02, P213_F05, P212_B12, P211_H07, P213_C11, P211_D03, P215_F06, P211_C05, P211_C06, P212_F11, P211_A06, P211_B10, P212_A12, P213_C02, P211_A03, P211_C10, P215_D05, P213_D04, P212_E06, P212_E12, P211_A10, P212_C01, P212_C08, P211_D12, P211_C07, P215_D06, P211_H09, P213_E05, P213_H03, P215_C01, P214_A10, P211_G09, P215_C07, P216_E06, P216_H01, P215_E11, P215_A07, D2M002-101 (P211_A12), D2M002-102 (P212_E04), D2M002-103 (P212_F01), D2M002-104 (P212_E01), D2M002-105 (P211_B12), D2M002-106 (P213_A03), D2M002-107 (P213_H05), D2M002-108 (P211_B09), D2M002-109 (P212_D03), D2M002-110 (P211_E08), D2M002-111, D2M002-112, D2M002-113, D2M002-114, D2M002-115, D2M002-116, D2M002-117, D2M002-118, D2M002-119, D2M002-120, D2M002-121, D2M002-122, D2M002-123, D2M002-124, D2M002-125, D2M002-126, D2M002-127, D2M002-128, D2M002-129, D2M002-130, D2M002-131, D2M002-132, D2M002-103HV1, D2M002-103HV2, D2M002-103LV1, D2M002-103LV3, D2M002-103LV4, D2M002-103LV5, D2M002-126LV1, D2M002-126LV2, chimeric antibodies thereof, and humanized antibodies thereof.

The CDR sequences for D2M002-001, and D2M002-001 derived antibodies (e.g., humanized antibodies) include CDRs of the heavy chain variable domain, SEQ ID NOs: 7-9, and CDRs of the light chain variable domain, SEQ ID NOs: 10-12 as defined by Kabat numbering. The CDRs can also be defined by Chothia system. Under the Chothia numbering, the CDR sequences of the heavy chain variable domain are set forth in SEQ ID NOs: 13-15, and CDR sequences of the light chain variable domain are set forth in SEQ ID NOs: 16-18.

Similarly, the CDR sequences for D2M002-002, and D2M002-002 derived antibodies include CDRs of the heavy chain variable domain, SEQ ID NOs: 37-39, and CDRs of the light chain variable domain, SEQ ID NOs: 40-42, as defined by Kabat numbering. Under Chothia numbering, the CDR sequences of the heavy chain variable domain are set forth in SEQ ID NOs: 43-45, and CDRs of the light chain variable domain are set forth in SEQ ID NOs: 46-48.

The CDR sequences for D2M002-003, and D2M002-003 derived antibodies include CDRs of the heavy chain variable domain, SEQ ID NOs: 67-69, and CDRs of the light chain variable domain, SEQ ID NOs: 70-72, as defined by Kabat numbering. Under Chothia numbering, the CDR sequences of the heavy chain variable domain are set forth in SEQ ID NOs: 73-75, and CDRs of the light chain variable domain are set forth in SEQ ID NOs: 76-78.

The CDR sequences for D2M002-004, and D2M002-004 derived antibodies include CDRs of the heavy chain variable domain, SEQ ID NOs: 97-99, and CDRs of the light chain variable domain, SEQ ID NOs: 100-102, as defined by Kabat numbering. Under Chothia numbering, the CDR sequences of the heavy chain variable domain are set forth in SEQ ID NOs: 103-105, and CDRs of the light chain variable domain are set forth in SEQ ID NOs: 106-108.

The CDR sequences for D2M002-008, and D2M002-008 derived antibodies include CDRs of the heavy chain variable domain, SEQ ID NOs: 127-129, and CDRs of the light chain variable domain, SEQ ID NOs: 130-132, as defined by Kabat numbering. Under Chothia numbering, the CDR sequences of the heavy chain variable domain are set forth in SEQ ID NOs: 133-135, and CDRs of the light chain variable domain are set forth in SEQ ID NOs: 136-138.

The CDR sequences for D2M002-011, and D2M002-011 derived antibodies include CDRs of the heavy chain variable domain, SEQ ID NOs: 157-159, and CDRs of the light chain variable domain, SEQ ID NOs: 160-162, as defined by Kabat numbering. Under Chothia numbering, the CDR sequences of the heavy chain variable domain are set forth in SEQ ID NOs: 163-165, and CDRs of the light chain variable domain are set forth in SEQ ID NOs: 166-168.

The CDR sequences for D2M002-012, and D2M002-012 derived antibodies include CDRs of the heavy chain variable domain, SEQ ID NOs: 187-189, and CDRs of the light chain variable domain, SEQ ID NOs: 190-192, as defined by Kabat numbering. Under Chothia numbering, the CDR sequences of the heavy chain variable domain are set forth in SEQ ID NOs: 193-195, and CDRs of the light chain variable domain are set forth in SEQ ID NOs: 196-198.

The CDR sequences for D2M002-016, and D2M002-016 derived antibodies include CDRs of the heavy chain variable domain, SEQ ID NOs: 217-219, and CDRs of the light chain variable domain, SEQ ID NOs: 220-222, as defined by Kabat numbering. Under Chothia numbering, the CDR sequences of the heavy chain variable domain are set forth in SEQ ID NOs: 223-225, and CDRs of the light chain variable domain are set forth in SEQ ID NOs: 226-228.

The CDR sequences for D2M002-017, and D2M002-017 derived antibodies include CDRs of the heavy chain variable domain, SEQ ID NOs: 247-249, and CDRs of the light chain variable domain, SEQ ID NOs: 250-252, as defined by Kabat numbering. Under Chothia numbering, the CDR sequences of the heavy chain variable domain are set forth in SEQ ID NOs: 253-255, and CDRs of the light chain variable domain are set forth in SEQ ID NOs: 256-258.

The CDR sequences for D2M002-018, and D2M002-018 derived antibodies include CDRs of the heavy chain variable domain, SEQ ID NOs: 277-279, and CDRs of the light chain variable domain, SEQ ID NOs: 280-282, as defined by Kabat numbering. Under Chothia numbering, the CDR sequences of the heavy chain variable domain are set forth in SEQ ID NOs: 283-285, and CDRs of the light chain variable domain are set forth in SEQ ID NOs: 286-288.

The CDR sequences for D2M002-020, and D2M002-020 derived antibodies include CDRs of the heavy chain variable domain, SEQ ID NOs: 307-309, and CDRs of the light chain variable domain, SEQ ID NOs: 310-312, as defined by Kabat numbering. Under Chothia numbering, the CDR sequences of the heavy chain variable domain are set forth in SEQ ID NOs: 313-315, and CDRs of the light chain variable domain are set forth in SEQ ID NOs: 316-318.

The CDR sequences for D2M002-021, and D2M002-021 derived antibodies include CDRs of the heavy chain variable domain, SEQ ID NOs: 337-339, and CDRs of the light chain variable domain, SEQ ID NOs: 340-342, as defined by Kabat numbering. Under Chothia numbering, the CDR sequences of the heavy chain variable domain are set forth in SEQ ID NOs: 343-345, and CDRs of the light chain variable domain are set forth in SEQ ID NOs: 346-348.

The CDR sequences for D2M002-022, and D2M002-022 derived antibodies include CDRs of the heavy chain variable domain, SEQ ID NOs: 367-369, and CDRs of the light chain variable domain, SEQ ID NOs: 370-372, as defined by Kabat numbering. Under Chothia numbering, the CDR sequences of the heavy chain variable domain are set forth in SEQ ID NOs: 373-375, and CDRs of the light chain variable domain are set forth in SEQ ID NOs: 376-378.

The CDR sequences for D2M002-023, and D2M002-023 derived antibodies include CDRs of the heavy chain variable domain, SEQ ID NOs: 397-399, and CDRs of the light chain variable domain, SEQ ID NOs: 400-402, as defined by Kabat numbering. Under Chothia numbering, the CDR sequences of the heavy chain variable domain are set forth in SEQ ID NOs: 403-405, and CDRs of the light chain variable domain are set forth in SEQ ID NOs: 406-408.

The CDR sequences for D2M002-026, and D2M002-026 derived antibodies include CDRs of the heavy chain variable domain, SEQ ID NOs: 427-429, and CDRs of the light chain variable domain, SEQ ID NOs: 430-432, as defined by Kabat numbering. Under Chothia numbering, the CDR sequences of the heavy chain variable domain are set forth in SEQ ID NOs: 433-435, and CDRs of the light chain variable domain are set forth in SEQ ID NOs: 436-438.

The CDR sequences for D2M002-027, and D2M002-027 derived antibodies include CDRs of the heavy chain variable domain, SEQ ID NOs: 457-459, and CDRs of the light chain variable domain, SEQ ID NOs: 460-462, as defined by Kabat numbering. Under Chothia numbering, the CDR sequences of the heavy chain variable domain are set forth in SEQ ID NOs: 463-465, and CDRs of the light chain variable domain are set forth in SEQ ID NOs: 466-468.

The CDR sequences for D2M002-028, and D2M002-028 derived antibodies include CDRs of the heavy chain variable domain, SEQ ID NOs: 487-489, and CDRs of the light chain variable domain, SEQ ID NOs: 490-492, as defined by Kabat numbering. Under Chothia numbering, the CDR sequences of the heavy chain variable domain are set forth in SEQ ID NOs: 493-495, and CDRs of the light chain variable domain are set forth in SEQ ID NOs: 496-498.

The CDR sequences for D2M002-029, and D2M002-029 derived antibodies include CDRs of the heavy chain variable domain, SEQ ID NOs: 517-519, and CDRs of the light chain variable domain, SEQ ID NOs: 520-522, as defined by Kabat numbering. Under Chothia numbering, the CDR sequences of the heavy chain variable domain are set forth in SEQ ID NOs: 523-525, and CDRs of the light chain variable domain are set forth in SEQ ID NOs: 526-528.

The CDR sequences for D2M002-031, and D2M002-031 derived antibodies include CDRs of the heavy chain variable domain, SEQ ID NOs: 547-549, and CDRs of the light chain variable domain, SEQ ID NOs: 550-552, as defined by Kabat numbering. Under Chothia numbering, the CDR sequences of the heavy chain variable domain are set forth in SEQ ID NOs: 553-555, and CDRs of the light chain variable domain are set forth in SEQ ID NOs: 556-558.

The CDR sequences for D2M002-035, and D2M002-035 derived antibodies include CDRs of the heavy chain variable domain, SEQ ID NOs: 577-579, and CDRs of the light chain variable domain, SEQ ID NOs: 580-582, as defined by Kabat numbering. Under Chothia numbering, the CDR sequences of the heavy chain variable domain are set forth in SEQ ID NOs: 583-585, and CDRs of the light chain variable domain are set forth in SEQ ID NOs: 586-588.

The CDR sequences for D2M002-036, and D2M002-036 derived antibodies include CDRs of the heavy chain variable domain, SEQ ID NOs: 607-609, and CDRs of the light chain variable domain, SEQ ID NOs: 610-612, as defined by Kabat numbering. Under Chothia numbering, the CDR sequences of the heavy chain variable domain are set forth in SEQ ID NOs: 613-615, and CDRs of the light chain variable domain are set forth in SEQ ID NOs: 616-618.

The CDR sequences for D2M002-040, and D2M002-040 derived antibodies include CDRs of the heavy chain variable domain, SEQ ID NOs: 637-639, and CDRs of the light chain variable domain, SEQ ID NOs: 640-642, as defined by Kabat numbering. Under Chothia numbering, the CDR sequences of the heavy chain variable domain are set forth in SEQ ID NOs: 643-645, and CDRs of the light chain variable domain are set forth in SEQ ID NOs: 646-648.

The CDR sequences for D2M002-041, and D2M002-041 derived antibodies include CDRs of the heavy chain variable domain, SEQ ID NOs: 667-669, and CDRs of the light chain variable domain, SEQ ID NOs: 670-672, as defined by Kabat numbering. Under Chothia numbering, the CDR sequences of the heavy chain variable domain are set forth in SEQ ID NOs: 673-675, and CDRs of the light chain variable domain are set forth in SEQ ID NOs: 676-678.

The CDR sequences for D2M002-044, and D2M002-044 derived antibodies include CDRs of the heavy chain variable domain, SEQ ID NOs: 697-699, and CDRs of the light chain variable domain, SEQ ID NOs: 700-702, as defined by Kabat numbering. Under Chothia numbering, the CDR sequences of the heavy chain variable domain are set forth in SEQ ID NOs: 703-705, and CDRs of the light chain variable domain are set forth in SEQ ID NOs: 706-708.

The CDR sequences for D2M002-045, and D2M002-045 derived antibodies include CDRs of the heavy chain variable domain, SEQ ID NOs: 727-729, and CDRs of the light chain variable domain, SEQ ID NOs: 730-732, as defined by Kabat numbering. Under Chothia numbering, the CDR sequences of the heavy chain variable domain are set forth in SEQ ID NOs: 733-735, and CDRs of the light chain variable domain are set forth in SEQ ID NOs: 736-738.

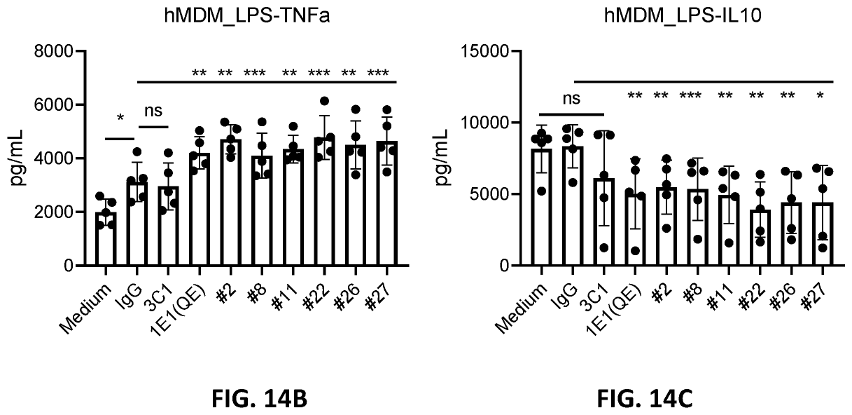

The VH and VL CDR sequences of the above antibodies under IMGT, North, or Aho numberings are also provided in FIG. 14.

In some embodiments, the antibodies and antigen-binding fragments described herein can be categorized as a D2M002-002 lineage, e.g., D2M002-002, D2M002-103, D2M002-103HV1, D2M002-103HV2, D2M002-103LV1, D2M002-103LV3, D2M002-103LV4, and D2M002-103LV5. In some embodiments, the antibodies and antigen-binding fragments described herein can be categorized as a D2M002-026 lineage, e.g., D2M002-026, D2M002-114, D2M002-115, D2M002-116, D2M002-117, D2M002-118, D2M002-119, D2M002-120, D2M002-121, D2M002-122, D2M002-123, D2M002-124, D2M002-125, D2M002-126, D2M002-127, D2M002-128, D2M002-129, D2M002-130, D2M002-131, D2M002-132, D2M002-126LV1, and D2M002-126LV2. In some embodiments, the above antibodies or variants have humanized and/or optimized sequences.

The CDR sequences for D2M002-103, and D2M002-103 derived antibodies include CDRs of the heavy chain variable domain, SEQ ID NOs: 1069-1071, and CDRs of the light chain variable domain, SEQ ID NOs: 1072-1074, as defined by Kabat numbering. Under Chothia numbering, the CDR sequences of the heavy chain variable domain are set forth in SEQ ID NOs: 1075-1077, and CDRs of the light chain variable domain are set forth in SEQ ID NOs: 1078-1080.

The CDR sequences for D2M002-103HV1, and D2M002-103HV1 derived antibodies include CDRs of the heavy chain variable domain, SEQ ID NOs: 1093-1095, and CDRs of the light chain variable domain, SEQ ID NOs: 1096-1098, as defined by Kabat numbering. Under Chothia numbering, the CDR sequences of the heavy chain variable domain are set forth in SEQ ID NOs: 1099-1101, and CDRs of the light chain variable domain are set forth in SEQ ID NOs: 1102-1104.

The CDR sequences for D2M002-103HV2, and D2M002-103HV2 derived antibodies include CDRs of the heavy chain variable domain, SEQ ID NOs: 1117-1119, and CDRs of the light chain variable domain, SEQ ID NOs: 1120-1122, as defined by Kabat numbering. Under Chothia numbering, the CDR sequences of the heavy chain variable domain are set forth in SEQ ID NOs: 1123-1125, and CDRs of the light chain variable domain are set forth in SEQ ID NOs: 1126-1128.

The CDR sequences for D2M002-103LV1, and D2M002-103LV1 derived antibodies include CDRs of the heavy chain variable domain, SEQ ID NOs: 1141-1143, and CDRs of the light chain variable domain, SEQ ID NOs: 1144-1146, as defined by Kabat numbering. Under Chothia numbering, the CDR sequences of the heavy chain variable domain are set forth in SEQ ID NOs: 1147-1149, and CDRs of the light chain variable domain are set forth in SEQ ID NOs: 1150-1152.

The CDR sequences for D2M002-103LV3, and D2M002-103LV3 derived antibodies include CDRs of the heavy chain variable domain, SEQ ID NOs: 1165-1167, and CDRs of the light chain variable domain, SEQ ID NOs: 1168-1170, as defined by Kabat numbering. Under Chothia numbering, the CDR sequences of the heavy chain variable domain are set forth in SEQ ID NOs: 1171-1173, and CDRs of the light chain variable domain are set forth in SEQ ID NOs: 1174-1176.

The CDR sequences for D2M002-103LV4, and D2M002-103LV4 derived antibodies include CDRs of the heavy chain variable domain, SEQ ID NOs: 1189-1191, and CDRs of the light chain variable domain, SEQ ID NOs: 1192-1194, as defined by Kabat numbering. Under Chothia numbering, the CDR sequences of the heavy chain variable domain are set forth in SEQ ID NOs: 1195-1197, and CDRs of the light chain variable domain are set forth in SEQ ID NOs: 1198-1200.

The CDR sequences for D2M002-103LV5, and D2M002-103LV5 derived antibodies include CDRs of the heavy chain variable domain, SEQ ID NOs: 1213-1215, and CDRs of the light chain variable domain, SEQ ID NOs: 1216-1218, as defined by Kabat numbering. Under Chothia numbering, the CDR sequences of the heavy chain variable domain are set forth in SEQ ID NOs: 1219-1221, and CDRs of the light chain variable domain are set forth in SEQ ID NOs: 1222-1224.

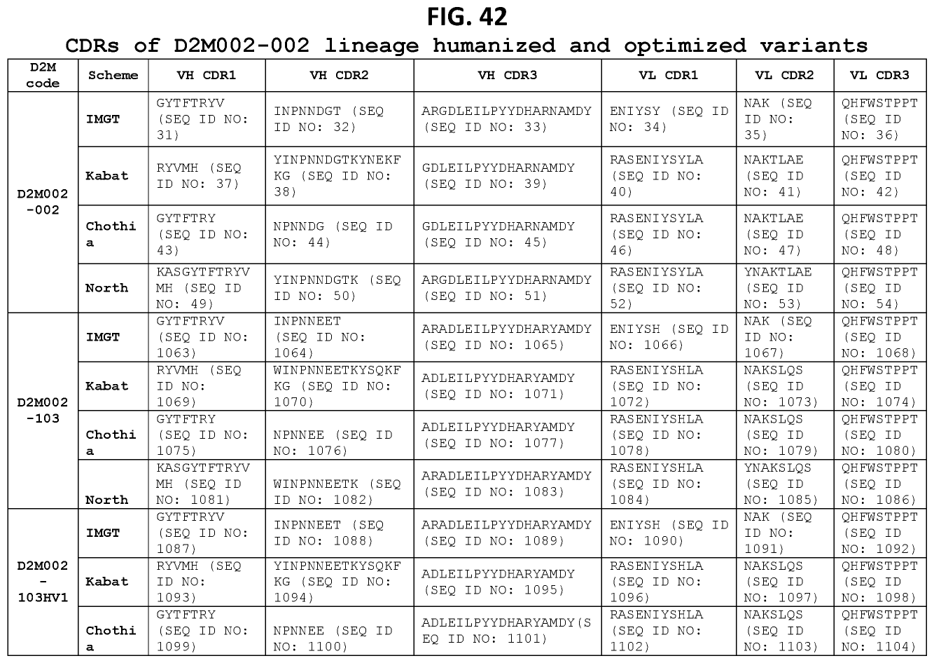

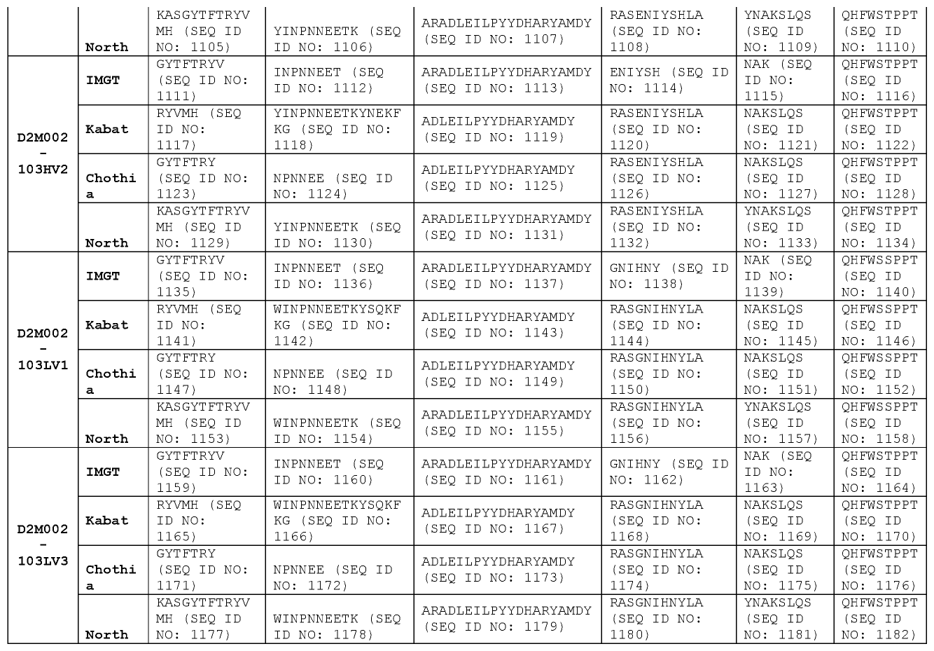

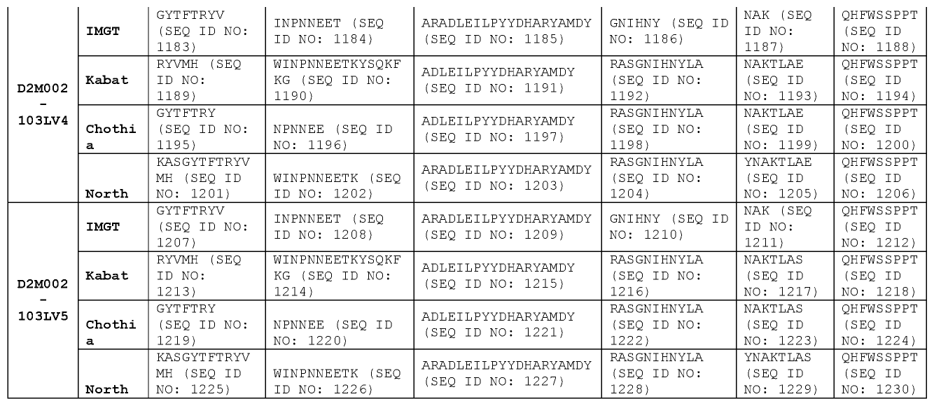

The VH and VL CDR sequences of the above antibodies (e.g., D2M002-002 lineage humanized and/or optimized variants) under IMGT or North numberings are also provided in FIG. 42.

The CDR sequences for D2M002-114, and D2M002-114 derived antibodies include CDRs of the heavy chain variable domain, SEQ ID NOs: 1237-1239, and CDRs of the light chain variable domain, SEQ ID NOs: 1240-1242, as defined by Kabat numbering. Under Chothia numbering, the CDR sequences of the heavy chain variable domain are set forth in SEQ ID NOs: 1243-1245, and CDRs of the light chain variable domain are set forth in SEQ ID NOs: 1246-1248.

The CDR sequences for D2M002-115, and D2M002-115 derived antibodies include CDRs of the heavy chain variable domain, SEQ ID NOs: 1261-1263, and CDRs of the light chain variable domain, SEQ ID NOs: 1264-1266, as defined by Kabat numbering. Under Chothia numbering, the CDR sequences of the heavy chain variable domain are set forth in SEQ ID NOs: 1267-1269, and CDRs of the light chain variable domain are set forth in SEQ ID NOs: 1270-1272.

The CDR sequences for D2M002-116, and D2M002-116 derived antibodies include CDRs of the heavy chain variable domain, SEQ ID NOs: 1285-1287, and CDRs of the light chain variable domain, SEQ ID NOs: 1288-1290, as defined by Kabat numbering. Under Chothia numbering, the CDR sequences of the heavy chain variable domain are set forth in SEQ ID NOs: 1291-1293, and CDRs of the light chain variable domain are set forth in SEQ ID NOs: 1294-1296.

The CDR sequences for D2M002-117, and D2M002-117 derived antibodies include CDRs of the heavy chain variable domain, SEQ ID NOs: 1309-1311, and CDRs of the light chain variable domain, SEQ ID NOs: 1312-1314, as defined by Kabat numbering. Under Chothia numbering, the CDR sequences of the heavy chain variable domain are set forth in SEQ ID NOs: 1315-1317, and CDRs of the light chain variable domain are set forth in SEQ ID NOs: 1318-1320.

The CDR sequences for D2M002-118, and D2M002-118 derived antibodies include CDRs of the heavy chain variable domain, SEQ ID NOs: 1333-1335, and CDRs of the light chain variable domain, SEQ ID NOs: 1336-1338, as defined by Kabat numbering. Under Chothia numbering, the CDR sequences of the heavy chain variable domain are set forth in SEQ ID NOs: 1339-1341, and CDRs of the light chain variable domain are set forth in SEQ ID NOs: 1342-1344.

The CDR sequences for D2M002-119, and D2M002-119 derived antibodies include CDRs of the heavy chain variable domain, SEQ ID NOs: 1357-1359, and CDRs of the light chain variable domain, SEQ ID NOs: 1360-1362, as defined by Kabat numbering. Under Chothia numbering, the CDR sequences of the heavy chain variable domain are set forth in SEQ ID NOs: 1362-1365, and CDRs of the light chain variable domain are set forth in SEQ ID NOs: 1366-1368.

The CDR sequences for D2M002-120, and D2M002-120 derived antibodies include CDRs of the heavy chain variable domain, SEQ ID NOs: 1381-1383, and CDRs of the light chain variable domain, SEQ ID NOs: 1384-1386, as defined by Kabat numbering. Under Chothia numbering, the CDR sequences of the heavy chain variable domain are set forth in SEQ ID NOs: 1387-1389, and CDRs of the light chain variable domain are set forth in SEQ ID NOs: 1390-1392.

The CDR sequences for D2M002-121, and D2M002-121 derived antibodies include CDRs of the heavy chain variable domain, SEQ ID NOs: 1405-1407, and CDRs of the light chain variable domain, SEQ ID NOs: 1408-1410, as defined by Kabat numbering. Under Chothia numbering, the CDR sequences of the heavy chain variable domain are set forth in SEQ ID NOs: 1411-1413, and CDRs of the light chain variable domain are set forth in SEQ ID NOs: 1414-1416.

The CDR sequences for D2M002-122, and D2M002-122 derived antibodies include CDRs of the heavy chain variable domain, SEQ ID NOs: 1429-1431, and CDRs of the light chain variable domain, SEQ ID NOs: 1432-1434, as defined by Kabat numbering. Under Chothia numbering, the CDR sequences of the heavy chain variable domain are set forth in SEQ ID NOs: 1435-1437, and CDRs of the light chain variable domain are set forth in SEQ ID NOs: 1438-1440.

The CDR sequences for D2M002-123, and D2M002-123 derived antibodies include CDRs of the heavy chain variable domain, SEQ ID NOs: 1453-1455, and CDRs of the light chain variable domain, SEQ ID NOs: 1456-1458, as defined by Kabat numbering. Under Chothia numbering, the CDR sequences of the heavy chain variable domain are set forth in SEQ ID NOs: 1459-1461, and CDRs of the light chain variable domain are set forth in SEQ ID NOs: 1462-1464.

The CDR sequences for D2M002-124, and D2M002-124 derived antibodies include CDRs of the heavy chain variable domain, SEQ ID NOs: 1477-1479, and CDRs of the light chain variable domain, SEQ ID NOs: 1480-1482, as defined by Kabat numbering. Under Chothia numbering, the CDR sequences of the heavy chain variable domain are set forth in SEQ ID NOs: 1483-1485, and CDRs of the light chain variable domain are set forth in SEQ ID NOs: 1486-1488.

The CDR sequences for D2M002-125, and D2M002-125 derived antibodies include CDRs of the heavy chain variable domain, SEQ ID NOs: 1501-1503, and CDRs of the light chain variable domain, SEQ ID NOs: 1504-1506, as defined by Kabat numbering. Under Chothia numbering, the CDR sequences of the heavy chain variable domain are set forth in SEQ ID NOs: 1507-1509, and CDRs of the light chain variable domain are set forth in SEQ ID NOs: 1510-1512.

The CDR sequences for D2M002-126, and D2M002-126 derived antibodies include CDRs of the heavy chain variable domain, SEQ ID NOs: 1525-1527, and CDRs of the light chain variable domain, SEQ ID NOs: 1528-1530, as defined by Kabat numbering. Under Chothia numbering, the CDR sequences of the heavy chain variable domain are set forth in SEQ ID NOs: 1531-1533, and CDRs of the light chain variable domain are set forth in SEQ ID NOs: 1534-1536.

The CDR sequences for D2M002-127, and D2M002-127 derived antibodies include CDRs of the heavy chain variable domain, SEQ ID NOs: 1549-1551, and CDRs of the light chain variable domain, SEQ ID NOs: 1552-1554, as defined by Kabat numbering. Under Chothia numbering, the CDR sequences of the heavy chain variable domain are set forth in SEQ ID NOs: 1555-1557, and CDRs of the light chain variable domain are set forth in SEQ ID NOs: 1558-1560.

The CDR sequences for D2M002-128, and D2M002-128 derived antibodies include CDRs of the heavy chain variable domain, SEQ ID NOs: 1573-1575, and CDRs of the light chain variable domain, SEQ ID NOs: 1576-1578, as defined by Kabat numbering. Under Chothia numbering, the CDR sequences of the heavy chain variable domain are set forth in SEQ ID NOs: 1579-1581, and CDRs of the light chain variable domain are set forth in SEQ ID NOs: 1582-1584.

The CDR sequences for D2M002-129, and D2M002-129 derived antibodies include CDRs of the heavy chain variable domain, SEQ ID NOs: 1597-1599, and CDRs of the light chain variable domain, SEQ ID NOs: 1600-1602, as defined by Kabat numbering. Under Chothia numbering, the CDR sequences of the heavy chain variable domain are set forth in SEQ ID NOs: 1603-1605, and CDRs of the light chain variable domain are set forth in SEQ ID NOs: 1606-1608.

The CDR sequences for D2M002-130, and D2M002-130 derived antibodies include CDRs of the heavy chain variable domain, SEQ ID NOs: 1621-1623, and CDRs of the light chain variable domain, SEQ ID NOs: 1624-1636, as defined by Kabat numbering. Under Chothia numbering, the CDR sequences of the heavy chain variable domain are set forth in SEQ ID NOs: 1627-1629, and CDRs of the light chain variable domain are set forth in SEQ ID NOs: 1630-1632.

The CDR sequences for D2M002-131, and D2M002-131 derived antibodies include CDRs of the heavy chain variable domain, SEQ ID NOs: 1645-1647, and CDRs of the light chain variable domain, SEQ ID NOs: 1648-1650, as defined by Kabat numbering. Under Chothia numbering, the CDR sequences of the heavy chain variable domain are set forth in SEQ ID NOs: 1651-1653, and CDRs of the light chain variable domain are set forth in SEQ ID NOs: 1654-1656.

The CDR sequences for D2M002-132, and D2M002-132 derived antibodies include CDRs of the heavy chain variable domain, SEQ ID NOs: 1669-1671, and CDRs of the light chain variable domain, SEQ ID NOs: 1672-1674, as defined by Kabat numbering. Under Chothia numbering, the CDR sequences of the heavy chain variable domain are set forth in SEQ ID NOs: 1675-1677, and CDRs of the light chain variable domain are set forth in SEQ ID NOs: 1678-1680.

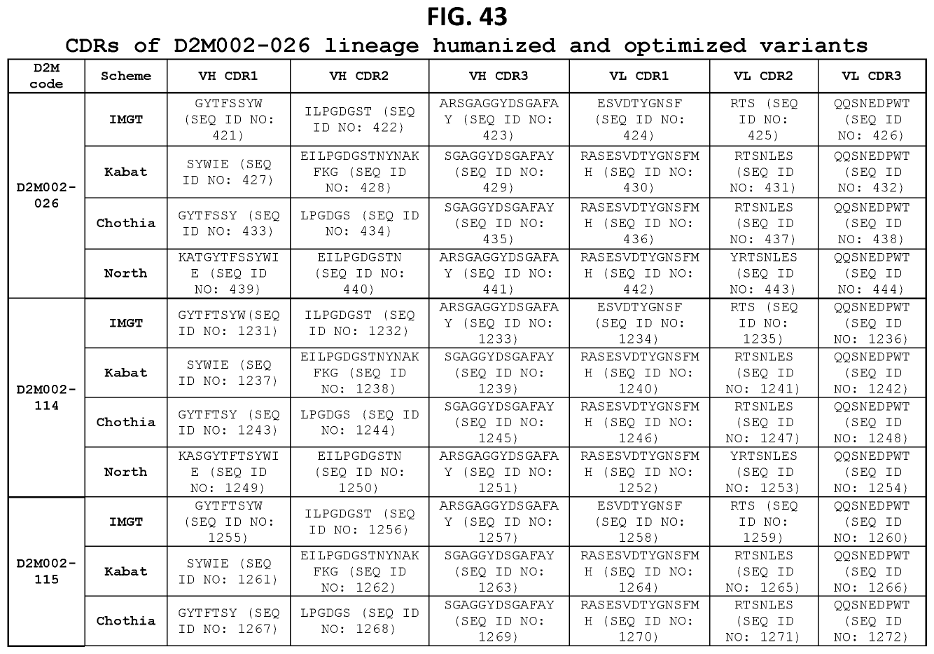

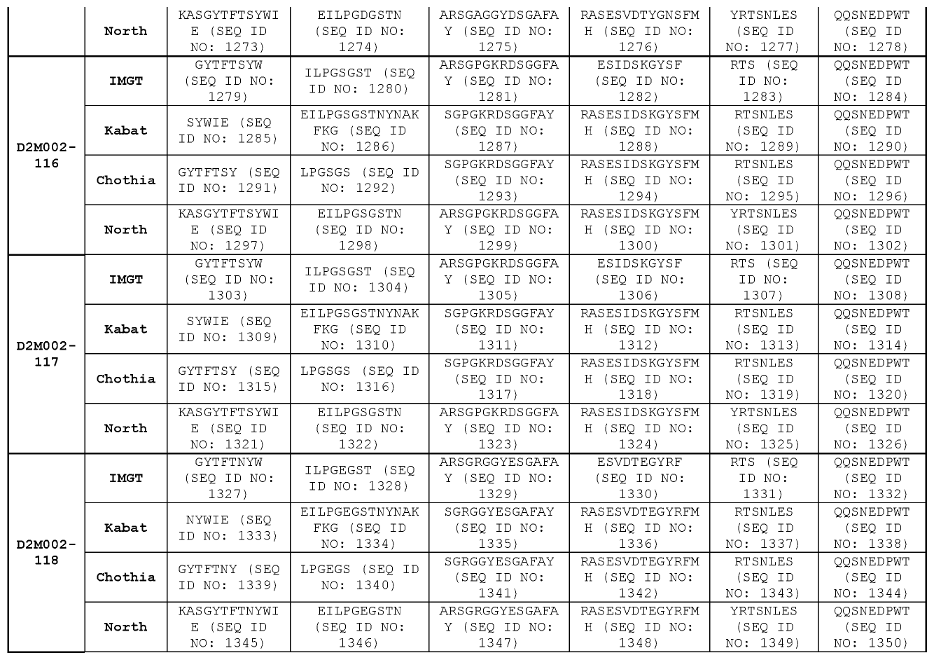

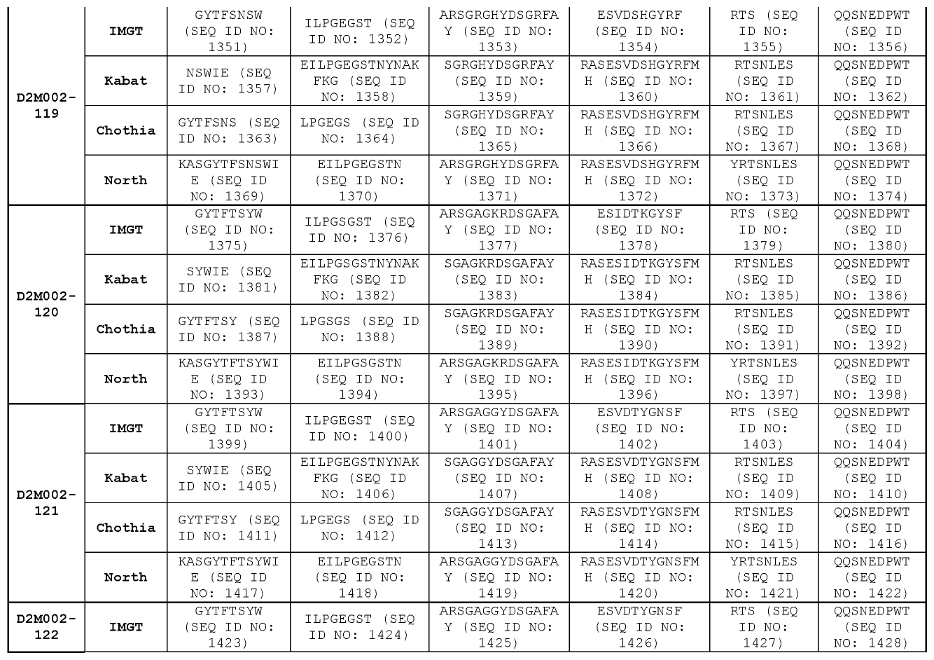

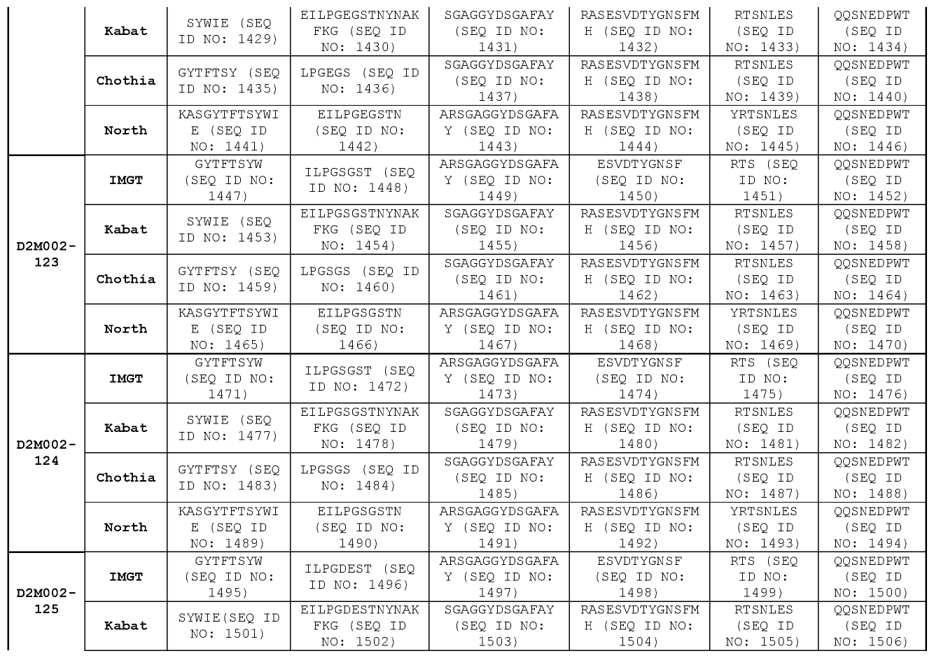

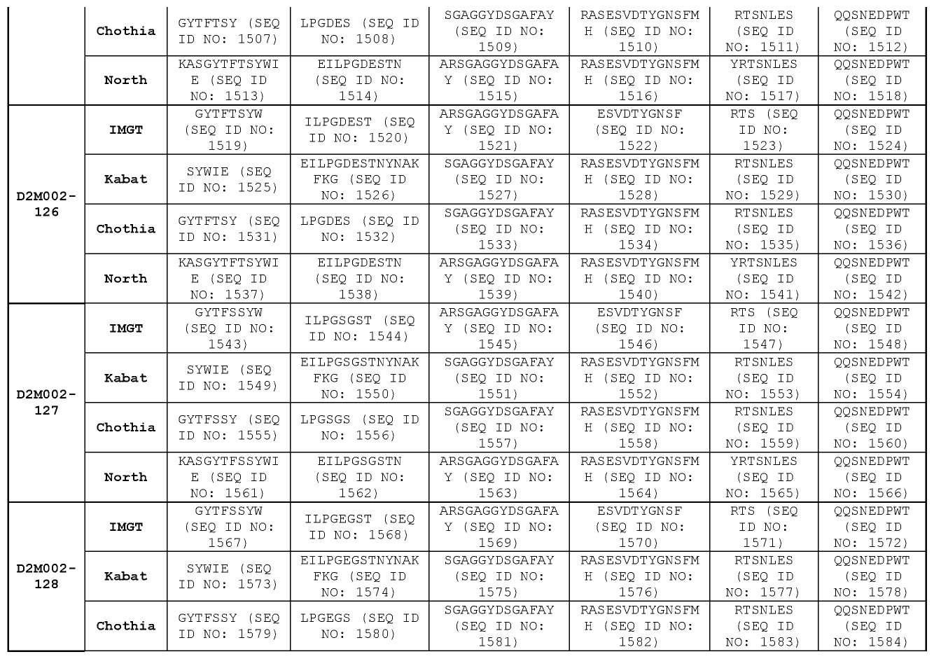

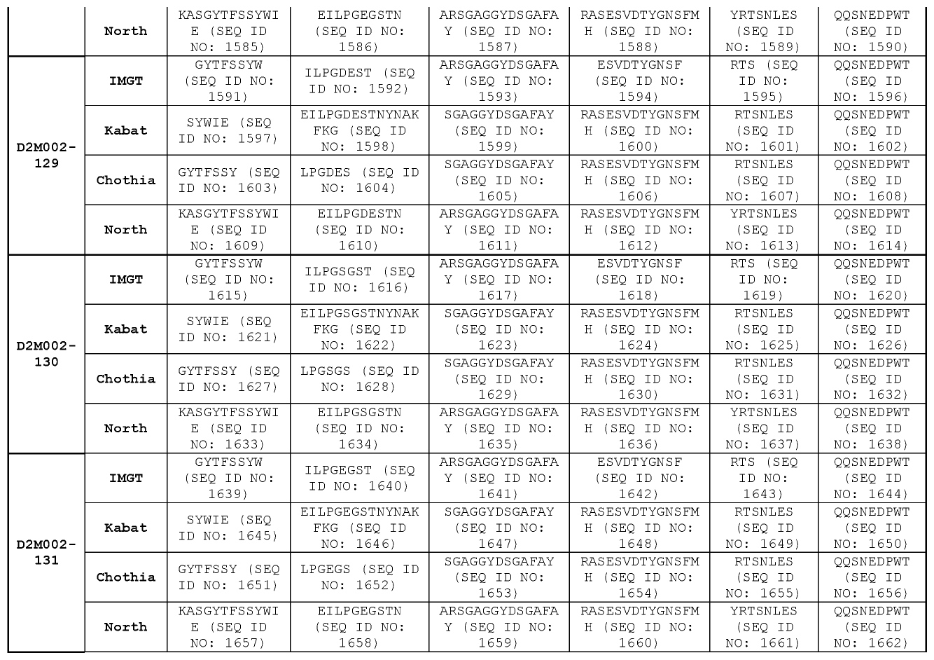

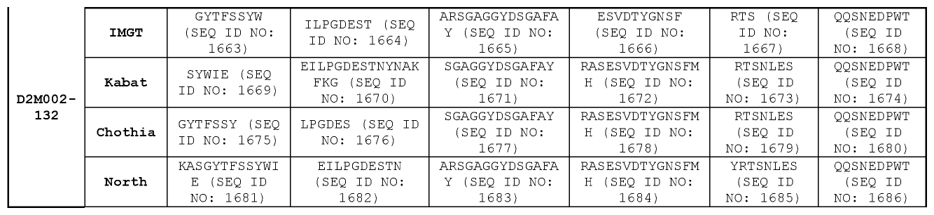

The VH and VL CDR sequences of the above antibodies (e.g., D2M002-026 lineage humanized and/or optimized variants) under IMGT or North numberings are also provided in FIG. 43. The amino acid sequence for heavy chain variable region and light variable region of humanized antibodies are also provided. The amino acid sequence for the heavy chain variable region (VH) of D2M002-001 is set forth in SEQ ID NO: 751. The amino acid sequence for the light chain variable region (VL) of D2M002-001 is set forth in SEQ ID NO: 752. The amino acid sequence for the VH of D2M002-002 is set forth in SEQ ID NO: 753. The amino acid sequence for the VL of D2M002-002 is set forth in SEQ ID NO: 754. The amino acid sequence for the VH of D2M002-003 is set forth in SEQ ID NO: 755. The amino acid sequence for the VL of D2M002-003 is set forth in SEQ ID NO: 756. The amino acid sequence for the VH of D2M002-004 is set forth in SEQ ID NO: 757. The amino acid sequence for the VL of D2M002-004 is set forth in SEQ ID NO: 758. The amino acid sequence for the VH of D2M002-008 is set forth in SEQ ID NO: 759. The amino acid sequence for the VL of D2M002-008 is set forth in SEQ ID NO: 760. The amino acid sequence for the VH of D2M002-011 is set forth in SEQ ID NO: 761. The amino acid sequence for the VL of D2M002-011 is set forth in SEQ ID NO: 762. The amino acid sequence for the VH of D2M002-012 is set forth in SEQ ID NO: 763. The amino acid sequence for the VL of D2M002-012 is set forth in SEQ ID NO: 764. The amino acid sequence for the VH of D2M002-016 is set forth in SEQ ID NO: 765. The amino acid sequence for the VL of D2M002-016 is set forth in SEQ ID NO: 766. The amino acid sequence for the VH of D2M002-017 is set forth in SEQ ID NO: 767. The amino acid sequence for the VL of D2M002-017 is set forth in SEQ ID NO: 768. The amino acid sequence for the VH of D2M002-018 is set forth in SEQ ID NO: 769. The amino acid sequence for the VL of D2M002-018 is set forth in SEQ ID NO: 770. The amino acid sequence for the VH of D2M002-020 is set forth in SEQ ID NO: 771. The amino acid sequence for the VL of D2M002-020 is set forth in SEQ ID NO: 772. The amino acid sequence for the VH of D2M002-021 is set forth in SEQ ID NO: 773. The amino acid sequence for the VL of D2M002-021 is set forth in SEQ ID NO: 774. The amino acid sequence for the VH of D2M002-022 is set forth in SEQ ID NO: 775. The amino acid sequence for the VL of D2M002-022 is set forth in SEQ ID NO: 776. The amino acid sequence for the VH of D2M002-023 is set forth in SEQ ID NO: 777. The amino acid sequence for the VL of D2M002-023 is set forth in SEQ ID NO: 778. The amino acid sequence for the VH of D2M002-026 is set forth in SEQ ID NO: 779. The amino acid sequence for the VL of D2M002-026 is set forth in SEQ ID NO: 780. The amino acid sequence for the VH of D2M002-027 is set forth in SEQ ID NO: 781. The amino acid sequence for the VL of D2M002-027 is set forth in SEQ ID NO: 782. The amino acid sequence for the VH of D2M002-028 is set forth in SEQ ID NO: 783. The amino acid sequence for the VL of D2M002-028 is set forth in SEQ ID NO: 784. The amino acid sequence for the VH of D2M002-029 is set forth in SEQ ID NO: 785. The amino acid sequence for the VL of D2M002-029 is set forth in SEQ ID NO: 786. The amino acid sequence for the VH of D2M002-031 is set forth in SEQ ID NO: 787. The amino acid sequence for the VL of D2M002-031 is set forth in SEQ ID NO: 788. The amino acid sequence for the VH of D2M002-035 is set forth in SEQ ID NO: 789. The amino acid sequence for the VL of D2M002-035 is set forth in SEQ ID NO: 790. The amino acid sequence for the VH of D2M002-036 is set forth in SEQ ID NO: 791. The amino acid sequence for the VL of D2M002-036 is set forth in SEQ ID NO: 792. The amino acid sequence for the VH of D2M002-040 is set forth in SEQ ID NO: 793. The amino acid sequence for the VL of D2M002-040 is set forth in SEQ ID NO: 794. The amino acid sequence for the VH of D2M002-041 is set forth in SEQ ID NO: 795. The amino acid sequence for the VL of D2M002-041 is set forth in SEQ ID NO: 796. The amino acid sequence for the VH of D2M002-044 is set forth in SEQ ID NO: 797. The amino acid sequence for the VL of D2M002-044 is set forth in SEQ ID NO: 798. The amino acid sequence for the VH of D2M002-045 is set forth in SEQ ID NO: 799. The amino acid sequence for the VL of D2M002-045 is set forth in SEQ ID NO: 800. The amino acid sequence for the VH of D2M002-002-H12 is set forth in SEQ ID NO: 841. The amino acid sequence for the VL of D2M002-002-H12 is set forth in SEQ ID NO: 842. The amino acid sequence for the VH of D2M002-002-H26 is set forth in SEQ ID NO: 843. The amino acid sequence for the VL of D2M002-002-H26 is set forth in SEQ ID NO: 844. The amino acid sequence for the VH of D2M002-026-H14 is set forth in SEQ ID NO: 845. The amino acid sequence for the VL of D2M002-026-H14 is set forth in SEQ ID NO: 846. The amino acid sequence for the VH of D2M002-026-H25 is set forth in SEQ ID NO: 847. The amino acid sequence for the VL of D2M002-026-H25 is set forth in SEQ ID NO: 848. The amino acid sequence for the VH of P215_C06 is set forth in SEQ ID NO: 849. The amino acid sequence for the VL of P215_C06 is set forth in SEQ ID NO: 850. The amino acid sequence for the VH of P215_H02 is set forth in SEQ ID NO: 851. The amino acid sequence for the VL of P215_H02 is set forth in SEQ ID NO: 852. The amino acid sequence for the VH of P212_E10 is set forth in SEQ ID NO: 853. The amino acid sequence for the VL of P212_E10 is set forth in SEQ ID NO: 854. The amino acid sequence for the VH of P211_E01 is set forth in SEQ ID NO: 855. The amino acid sequence for the VL of P211_E01 is set forth in SEQ ID NO: 856. The amino acid sequence for the VH of P211_B01 is set forth in SEQ ID NO: 857. The amino acid sequence for the VL of P211_B01 is set forth in SEQ ID NO: 858. The amino acid sequence for the VH of P211_G04 is set forth in SEQ ID NO: 859. The amino acid sequence for the VL of P211_G04 is set forth in SEQ ID NO: 860. The amino acid sequence for the VH of P211_C01 is set forth in SEQ ID NO: 861. The amino acid sequence for the VL of P211_C01 is set forth in SEQ ID NO: 862. The amino acid sequence for the VH of P212_C04 is set forth in SEQ ID NO: 863. The amino acid sequence for the VL of P212_C04 is set forth in SEQ ID NO: 864. The amino acid sequence for the VH of P212_F06 is set forth in SEQ ID NO: 865. The amino acid sequence for the VL of P212_F06 is set forth in SEQ ID NO: 866. The amino acid sequence for the VH of P211_B02 is set forth in SEQ ID NO: 867. The amino acid sequence for the VL of P211_B02 is set forth in SEQ ID NO: 868. The amino acid sequence for the VH of P213_C03 is set forth in SEQ ID NO: 869. The amino acid sequence for the VL of P213_C03 is set forth in SEQ ID NO: 870. The amino acid sequence for the VH of P211_C02 is set forth in SEQ ID NO: 871. The amino acid sequence for the VL of P211_C02 is set forth in SEQ ID NO: 872. The amino acid sequence for the VH of P211_C09 is set forth in SEQ ID NO: 873. The amino acid sequence for the VL of P211_C09 is set forth in SEQ ID NO: 874. The amino acid sequence for the VH of P212_A03 is set forth in SEQ ID NO: 875. The amino acid sequence for the VL of P212_A03 is set forth in SEQ ID NO: 876. The amino acid sequence for the VH of P212_B07 is set forth in SEQ ID NO: 877. The amino acid sequence for the VL of P212_B07 is set forth in SEQ ID NO: 878. The amino acid sequence for the VH of P213_C05 is set forth in SEQ ID NO: 879. The amino acid sequence for the VL of P213_C05 is set forth in SEQ ID NO: 880. The amino acid sequence for the VH of P213_H11 is set forth in SEQ ID NO: 881. The amino acid sequence for the VL of P213_H11 is set forth in SEQ ID NO: 882. The amino acid sequence for the VH of P211_E07 is set forth in SEQ ID NO: 883. The amino acid sequence for the VL of P211_E07 is set forth in SEQ ID NO: 884. The amino acid sequence for the VH of P213_G10 is set forth in SEQ ID NO: 885. The amino acid sequence for the VL of P213_G10 is set forth in SEQ ID NO: 886. The amino acid sequence for the VH of P212_E03 is set forth in SEQ ID NO: 887. The amino acid sequence for the VL of P212_E03 is set forth in SEQ ID NO: 888. The amino acid sequence for the VH of P212_G10 is set forth in SEQ ID NO: 889. The amino acid sequence for the VL of P212_G10 is set forth in SEQ ID NO: 890. The amino acid sequence for the VH of P215_A01 is set forth in SEQ ID NO: 891. The amino acid sequence for the VL of P215_A01 is set forth in SEQ ID NO: 892. The amino acid sequence for the VH of P211_B05 is set forth in SEQ ID NO: 893. The amino acid sequence for the VL of P211_B05 is set forth in SEQ ID NO: 894. The amino acid sequence for the VH of P215_H01 is set forth in SEQ ID NO: 895. The amino acid sequence for the VL of P215_H01 is set forth in SEQ ID NO: 896. The amino acid sequence for the VH of P212_D01 is set forth in SEQ ID NO: 897. The amino acid sequence for the VL of P212_D01 is set forth in SEQ ID NO: 898. The amino acid sequence for the VH of P215_F02 is set forth in SEQ ID NO: 899. The amino acid sequence for the VL of P215_F02 is set forth in SEQ ID NO: 900. The amino acid sequence for the VH of P212_H04 is set forth in SEQ ID NO: 901. The amino acid sequence for the VL of P212_H04 is set forth in SEQ ID NO: 902. The amino acid sequence for the VH of P212_D04 is set forth in SEQ ID NO: 903. The amino acid sequence for the VL of P212_D04 is set forth in SEQ ID NO: 904. The amino acid sequence for the VH of P211_E02 is set forth in SEQ ID NO: 905. The amino acid sequence for the VL of P211_E02 is set forth in SEQ ID NO: 906. The amino acid sequence for the VH of P213_F05 is set forth in SEQ ID NO: 907. The amino acid sequence for the VL of P213_F05 is set forth in SEQ ID NO: 908. The amino acid sequence for the VH of P212_B12 is set forth in SEQ ID NO: 909. The amino acid sequence for the VL of P212_B12 is set forth in SEQ ID NO: 910. The amino acid sequence for the VH of P211_H07 is set forth in SEQ ID NO: 911. The amino acid sequence for the VL of P211_H07 is set forth in SEQ ID NO: 912. The amino acid sequence for the VH of P213_C11 is set forth in SEQ ID NO: 913. The amino acid sequence for the VL of P213_C11 is set forth in SEQ ID NO: 914. The amino acid sequence for the VH of P211_D03 is set forth in SEQ ID NO: 915. The amino acid sequence for the VL of P211_D03 is set forth in SEQ ID NO: 916. The amino acid sequence for the VH of P215_F06 is set forth in SEQ ID NO: 917. The amino acid sequence for the VL of P215_F06 is set forth in SEQ ID NO: 918. The amino acid sequence for the VH of P211_C05 is set forth in SEQ ID NO: 919. The amino acid sequence for the VL of P211_C05 is set forth in SEQ ID NO: 920. The amino acid sequence for the VH of P211_C06 is set forth in SEQ ID NO: 921. The amino acid sequence for the VL of P211_C06 is set forth in SEQ ID NO: 922. The amino acid sequence for the VH of P212_F11 is set forth in SEQ ID NO: 923. The amino acid sequence for the VL of P212_F11 is set forth in SEQ ID NO: 924. The amino acid sequence for the VH of P211_A06 is set forth in SEQ ID NO: 925. The amino acid sequence for the VL of P211_A06 is set forth in SEQ ID NO: 926. The amino acid sequence for the VH of P211_B10 is set forth in SEQ ID NO: 927. The amino acid sequence for the VL of P211_B10 is set forth in SEQ ID NO: 928. The amino acid sequence for the VH of P212_A12 is set forth in SEQ ID NO: 929. The amino acid sequence for the VL of P212_A12 is set forth in SEQ ID NO: 930. The amino acid sequence for the VH of P213_C02 is set forth in SEQ ID NO: 931. The amino acid sequence for the VL of P213_C02 is set forth in SEQ ID NO: 932. The amino acid sequence for the VH of P211_A03 is set forth in SEQ ID NO: 933. The amino acid sequence for the VL of P211_A03 is set forth in SEQ ID NO: 934. The amino acid sequence for the VH of P211_C10 is set forth in SEQ ID NO: 935. The amino acid sequence for the VL of P211_C10 is set forth in SEQ ID NO: 936. The amino acid sequence for the VH of P215_D05 is set forth in SEQ ID NO: 937. The amino acid sequence for the VL of P215_D05 is set forth in SEQ ID NO: 938. The amino acid sequence for the VH of P213_D04 is set forth in SEQ ID NO: 939. The amino acid sequence for the VL of P213_D04 is set forth in SEQ ID NO: 940. The amino acid sequence for the VH of P212_E06 is set forth in SEQ ID NO: 941. The amino acid sequence for the VL of P212_E06 is set forth in SEQ ID NO: 942. The amino acid sequence for the VH of P212_E12 is set forth in SEQ ID NO: 943. The amino acid sequence for the VL of P212_E12 is set forth in SEQ ID NO: 944. The amino acid sequence for the VH of P211_A10 is set forth in SEQ ID NO: 945. The amino acid sequence for the VL of P211_A10 is set forth in SEQ ID NO: 946. The amino acid sequence for the VH of P212_C01 is set forth in SEQ ID NO: 947. The amino acid sequence for the VL of P212_C01 is set forth in SEQ ID NO: 948. The amino acid sequence for the VH of P212_C08 is set forth in SEQ ID NO: 949. The amino acid sequence for the VL of P212_C08 is set forth in SEQ ID NO: 950. The amino acid sequence for the VH of P211_D12 is set forth in SEQ ID NO: 951. The amino acid sequence for the VL of P211_D12 is set forth in SEQ ID NO: 952. The amino acid sequence for the VH of P211_C07 is set forth in SEQ ID NO: 953. The amino acid sequence for the VL of P211_C07 is set forth in SEQ ID NO: 954. The amino acid sequence for the VH of P215 D06 is set forth in SEQ ID NO: 955. The amino acid sequence for the VL of P215_D06 is set forth in SEQ ID NO: 956. The amino acid sequence for the VH of P211_H09 is set forth in SEQ ID NO: 957. The amino acid sequence for the VL of P211_H09 is set forth in SEQ ID NO: 958. The amino acid sequence for the VH of P213_E05 is set forth in SEQ ID NO: 959. The amino acid sequence for the VL of P213_E05 is set forth in SEQ ID NO: 960. The amino acid sequence for the VH of P213_H03 is set forth in SEQ ID NO: 961. The amino acid sequence for the VL of P213_H03 is set forth in SEQ ID NO: 962. The amino acid sequence for the VH of P215_C01 is set forth in SEQ ID NO: 963. The amino acid sequence for the VL of P215_C01 is set forth in SEQ ID NO: 964. The amino acid sequence for the VH of P214_A10 is set forth in SEQ ID NO: 965. The amino acid sequence for the VL of P214_A10 is set forth in SEQ ID NO: 966. The amino acid sequence for the VH of P211_G09 is set forth in SEQ ID NO: 967. The amino acid sequence for the VL of P211_G09 is set forth in SEQ ID NO: 968. The amino acid sequence for the VH of P215_C07 is set forth in SEQ ID NO: 969. The amino acid sequence for the VL of P215_C07 is set forth in SEQ ID NO: 970. The amino acid sequence for the VH of P216_E06 is set forth in SEQ ID NO: 971. The amino acid sequence for the VL of P216_E06 is set forth in SEQ ID NO: 972. The amino acid sequence for the VH of P216_H01 is set forth in SEQ ID NO: 973. The amino acid sequence for the VL of P216_H01 is set forth in SEQ ID NO: 974. The amino acid sequence for the VH of P215_E11 is set forth in SEQ ID NO: 975. The amino acid sequence for the VL of P215_E11 is set forth in SEQ ID NO: 976. The amino acid sequence for the VH of P215_A07 is set forth in SEQ ID NO: 977. The amino acid sequence for the VL of P215_A07 is set forth in SEQ ID NO: 978. The amino acid sequence for the VH of D2M002-101 is set forth in SEQ ID NO: 979. The amino acid sequence for the VL of D2M002-101 is set forth in SEQ ID NO: 980. The amino acid sequence for the VH of D2M002-101 is set forth in SEQ ID NO: 979. The amino acid sequence for the VL of D2M002-101 is set forth in SEQ ID NO: 980, 981, 982, 983, or 984. The amino acid sequence for the VH of D2M002-102 is set forth in SEQ ID NO: 985. The amino acid sequence for the VL of D2M002-102 is set forth in SEQ ID NO: 986. The amino acid sequence for the VH of D2M002-103 is set forth in SEQ ID NO: 987. The amino acid sequence for the VL of D2M002-103 is set forth in SEQ ID NO: 988, 989, 990, 991, or 992. The amino acid sequence for the VH of D2M002-104 is set forth in SEQ ID NO: 993. The amino acid sequence for the VL of D2M002-104 is set forth in SEQ ID NO: 994. The amino acid sequence for the VH of D2M002-105 is set forth in SEQ ID NO: 995. The amino acid sequence for the VL of D2M002-105 is set forth in SEQ ID NO: 996. The amino acid sequence for the VH of D2M002-106 is set forth in SEQ ID NO: 997. The amino acid sequence for the VL of D2M002-106 is set forth in SEQ ID NO: 998. The amino acid sequence for the VH of D2M002-107 is set forth in SEQ ID NO: 999. The amino acid sequence for the VL of D2M002-107 is set forth in SEQ ID NO: 1000, 1001, 1002, 1003, or 1004. The amino acid sequence for the VH of D2M002-108 is set forth in SEQ ID NO: 1005. The amino acid sequence for the VL of D2M002-108 is set forth in SEQ ID NO: 1006. The amino acid sequence for the VH of D2M002-109 is set forth in SEQ ID NO: 1007. The amino acid sequence for the VL of D2M002-109 is set forth in SEQ ID NO: 1008, 1009, or 1010.

The amino acid sequence for the VH of D2M002-110 is set forth in SEQ ID NO: 1011. The amino acid sequence for the VL of D2M002-110 is set forth in SEQ ID NO: 1012. The amino acid sequence for the VH of D2M002-111 is set forth in SEQ ID NO: 1013. The amino acid sequence for the VL of D2M002-111 is set forth in SEQ ID NO: 1014. The amino acid sequence for the VH of D2M002-112 is set forth in SEQ ID NO: 1015. The amino acid sequence for the VL of D2M002-112 is set forth in SEQ ID NO: 1016. The amino acid sequence for the VH of D2M002-113 is set forth in SEQ ID NO: 1017. The amino acid sequence for the VL of D2M002-113 is set forth in SEQ ID NO: 1018. The amino acid sequence for the VH of D2M002-114 is set forth in SEQ ID NO: 1019. The amino acid sequence for the VL of D2M002-114 is set forth in SEQ ID NO: 1020. The amino acid sequence for the VH of D2M002-115 is set forth in SEQ ID NO: 1021. The amino acid sequence for the VL of D2M002-115 is set forth in SEQ ID NO: 1022. The amino acid sequence for the VH of D2M002-116 is set forth in SEQ ID NO: 1023. The amino acid sequence for the VL of D2M002-116 is set forth in SEQ ID NO: 1024. The amino acid sequence for the VH of D2M002-117 is set forth in SEQ ID NO: 1025. The amino acid sequence for the VL of D2M002-117 is set forth in SEQ ID NO: 1026. The amino acid sequence for the VH of D2M002-118 is set forth in SEQ ID NO: 1027. The amino acid sequence for the VL of D2M002-118 is set forth in SEQ ID NO: 1028. The amino acid sequence for the VH of D2M002-119 is set forth in SEQ ID NO: 1029. The amino acid sequence for the VL of D2M002-119 is set forth in SEQ ID NO: 1030. The amino acid sequence for the VH of D2M002-120 is set forth in SEQ ID NO: 1031. The amino acid sequence for the VL of D2M002-120 is set forth in SEQ ID NO: 1032. The amino acid sequence for the VH of D2M002-121 is set forth in SEQ ID NO: 1033. The amino acid sequence for the VL of D2M002-121 is set forth in SEQ ID NO: 1034. The amino acid sequence for the VH of D2M002-122 is set forth in SEQ ID NO: 1035. The amino acid sequence for the VL of D2M002-122 is set forth in SEQ ID NO: 1036. The amino acid sequence for the VH of D2M002-123 is set forth in SEQ ID NO: 1037. The amino acid sequence for the VL of D2M002-123 is set forth in SEQ ID NO: 1038. The amino acid sequence for the VH of D2M002-124 is set forth in SEQ ID NO: 1039. The amino acid sequence for the VL of D2M002-124 is set forth in SEQ ID NO: 1040. The amino acid sequence for the VH of D2M002-125 is set forth in SEQ ID NO: 1041. The amino acid sequence for the VL of D2M002-125 is set forth in SEQ ID NO: 1042. The amino acid sequence for the VH of D2M002-126 is set forth in SEQ ID NO: 1043. The amino acid sequence for the VL of D2M002-126 is set forth in SEQ ID NO: 1044. The amino acid sequence for the VH of D2M002-127 is set forth in SEQ ID NO: 1045. The amino acid sequence for the VL of D2M002-127 is set forth in SEQ ID NO: 1046. The amino acid sequence for the VH of D2M002-128 is set forth in SEQ ID NO: 1047. The amino acid sequence for the VL of D2M002-128 is set forth in SEQ ID NO: 1048. The amino acid sequence for the VH of D2M002-129 is set forth in SEQ ID NO: 1049. The amino acid sequence for the VL of D2M002-129 is set forth in SEQ ID NO: 1050. The amino acid sequence for the VH of D2M002-130 is set forth in SEQ ID NO: 1051. The amino acid sequence for the VL of D2M002-130 is set forth in SEQ ID NO: 1052. The amino acid sequence for the VH of D2M002-131 is set forth in SEQ ID NO: 1053. The amino acid sequence for the VL of D2M002-131 is set forth in SEQ ID NO: 1054. The amino acid sequence for the VH of D2M002-132 is set forth in SEQ ID NO: 1055. The amino acid sequence for the VL of D2M002-132 is set forth in SEQ ID NO: 1056.

The amino acid sequence for the VH of D2M002-103HV1 is set forth in SEQ ID NO: 1687. The amino acid sequence for the VL of D2M002-103HV1 is set forth in SEQ ID NO: 988. The amino acid sequence for the VH of D2M002-103HV2 is set forth in SEQ ID NO: 1688. The amino acid sequence for the VL of D2M002-103HV2 is set forth in SEQ ID NO: 988. The amino acid sequence for the VH of D2M002-103LV1 is set forth in SEQ ID NO: 987. The amino acid sequence for the VL of D2M002-103LV1 is set forth in SEQ ID NO: 989. The amino acid sequence for the VH of D2M002-103LV3 is set forth in SEQ ID NO: 987. The amino acid sequence for the VL of D2M002-103LV3 is set forth in SEQ ID NO: 990. The amino acid sequence for the VH of D2M002-103LV4 is set forth in SEQ ID NO: 987. The amino acid sequence for the VL of D2M002-103LV4 is set forth in SEQ ID NO: 991. The amino acid sequence for the VH of D2M002-103LV5 is set forth in SEQ ID NO: 987. The amino acid sequence for the VL of D2M002-103LV5 is set forth in SEQ ID NO: 992. The amino acid sequence for the VH of D2M002-126LV1 is set forth in SEQ ID NO: 1043. The amino acid sequence for the VL of D2M002-126LV1 is set forth in SEQ ID NO: 1689. The amino acid sequence for the VH of D2M002-126LV2 is set forth in SEQ ID NO: 1043. The amino acid sequence for the VL of D2M002-126LV2 is set forth in SEQ ID NO: 1690.

Any of these heavy chain variable region sequences (SEQ ID NO: 751, 753, 755, 757, 759, 761, 763, 765, 767, 769, 771, 773, 775, 777, 779, 781, 783, 785, 787, 789, 791, 793, 795, 797, 799, 841, 843, 845, 847, 849, 851, 853, 855, 857, 859, 861, 863, 865, 867, 869, 871, 873, 875, 877, 879, 881, 883, 885, 887, 889, 891, 893, 895, 897, 899, 901, 903, 905, 907, 909, 911, 913, 915, 917, 919, 921, 923, 925, 927, 929, 931, 933, 935, 937, 939, 941, 943, 945, 947, 949, 951, 953, 955, 957, 959, 961, 963, 965, 967, 969, 971, 973, 975, 977, 979, 985, 987, 993, 995, 997, 999, 1005, 1007, 1011, 1013, 1015, 1017, 1019, 1021, 1023, 1025, 1027, 1029, 1031, 1033, 1035, 1037, 1039, 1041, 1043, 1045, 1047, 1049, 1051, 1053, 1055, 1067, or 1068) can be paired with any of these light chain variable region sequences (SEQ ID NO: 752, 754, 756, 758, 760, 762, 764, 766, 768, 770, 772, 774, 776, 778, 780, 782, 784, 786, 788, 790, 792, 794, 796, 798, 800, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 981, 982, 983, 984, 986, 988, 989, 990, 991, 992, 994, 996, 998, 1000, 1001, 1002, 1003, 1004, 1006, 1008, 1009, 1010, 1012, 1014, 1016, 1018, 1020, 1022, 1024, 1026, 1028, 1030, 1032, 1034, 1036, 1038, 1040, 1042, 1044, 1046, 1048, 1050, 1052, 1054, 1056, 1689, or 1690).

In some embodiments, humanization percentage means the percentage identity of the heavy chain or light chain variable region sequence as compared to human antibody sequences in International Immunogenetics Information System (IMGT) database. The top hit means that the heavy chain or light chain variable region sequence is closer to a particular species than to other species. For example, top hit to human means that the sequence is closer to human than to other species. Top hit to human and Macacafascicularis means that the sequence has the same percentage identity to the human sequence and the Macacafascicularis sequence, and these percentages identities are highest as compared to the sequences of other species. In some embodiments, humanization percentage is greater than 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art, and is described, e.g., in Jones, Tim D., et al. “The INNs and outs of antibody nonproprietary names.” MAbs. Vol. 8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects.

Furthermore, in some embodiments, the antibodies or antigen-binding fragments thereof described herein can also contain one, two, or three heavy chain variable region CDRs selected from the group of SEQ ID NOs: 7-9, SEQ ID NOs: 37-39, SEQ ID NOs: 67-69, SEQ ID NOs: 97-99, SEQ ID NOs: 127-129, SEQ ID NOs: 157-159, SEQ ID NOs: 187-189, SEQ ID NOs: 217-219, SEQ ID NOs: 247-249, SEQ ID NOs: 277-279, SEQ ID NOs: 307-309, SEQ ID NOs: 337-339, SEQ ID NOs: 367-369, SEQ ID NOs: 397-399, SEQ ID NOs: 427-429, SEQ ID NOs: 457-459, SEQ ID NOs: 487-489, SEQ ID NOs: 517-519, SEQ ID NOs: 547-549, SEQ ID NOs: 577-579, SEQ ID NOs: 607-609, SEQ ID NOs: 637-639, SEQ ID NOs: 667-669, SEQ ID NOs: 697-699, SEQ ID NOs: 727-729, SEQ ID NOs: 1069-1071, SEQ ID NOs: 1093-1095, SEQ ID NOs: 1117-1119, SEQ ID NOs: 1141-1143, SEQ ID NOs: 1165-1167, SEQ ID NOs: 1189-1191, SEQ ID NOs: 1213-1215, SEQ ID NOs: 1237-1239, SEQ ID NOs: 1261-1263, SEQ ID NOs: 1285-1287, SEQ ID NOs: 1309-1311, SEQ ID NOs: 1333-1335, SEQ ID NOs: 1357-1359, SEQ ID NOs: 1381-1383, SEQ ID NOs: 1405-1407, SEQ ID NOs: 1429-1431, SEQ ID NOs: 1453-1455, SEQ ID NOs: 1477-1479, SEQ ID NOs: 1501-1503, SEQ ID NOs: 1525-1527, SEQ ID NOs: 1549-1551, SEQ ID NOs: 1573-1575, SEQ ID NOs: 1597-1599, SEQ ID NOs: 1621-1623, SEQ ID NOs: 1645-1647, and SEQ ID NOs: 1669-1671; and/or one, two, or three light chain variable region CDRs selected from the group of SEQ ID NOs: 10-12, SEQ ID NOs: 40-42, SEQ ID NOs: 70-72, SEQ ID NOs: 100-102, SEQ ID NOs: 130-132, SEQ ID NOs: 160-162, SEQ ID NOs: 190-192, SEQ ID NOs: 220-222, SEQ ID NOs: 250-252, SEQ ID NOs: 280-282, SEQ ID NOs: 310-312, SEQ ID NOs: 340-342, SEQ ID NOs: 370-372, SEQ ID NOs: 400-402, SEQ ID NOs: 430-432, SEQ ID NOs: 460-462, SEQ ID NOs: 490-492, SEQ ID NOs: 520-522, SEQ ID NOs: 550-552, SEQ ID NOs: 580-582, SEQ ID NOs: 610-612, SEQ ID NOs: 640-642, SEQ ID NOs: 670-672, SEQ ID NOs: 700-702, SEQ ID NOs: 730-732, SEQ ID NOs: 1072-1074, SEQ ID NOs: 1096-1098, SEQ ID NOs: 1120-1122, SEQ ID NOs: 1144-1146, SEQ ID NOs: 1168-1170, SEQ ID NOs: 1192-1194, SEQ ID NOs: 1216-1218, SEQ ID NOs: 1240-1242, SEQ ID NOs: 1264-1266, SEQ ID NOs: 1288-1290, SEQ ID NOs: 1312-1314, SEQ ID NOs: 1336-1338, SEQ ID NOs: 1360-1362, SEQ ID NOs: 1384-1386, SEQ ID NOs: 1408-1410, SEQ ID NOs: 1432-1434, SEQ ID NOs: 1456-1458, SEQ ID NOs: 1480-1482, SEQ ID NOs: 1504-1506, SEQ ID NOs: 1528-1530, SEQ ID NOs: 1552-1554, SEQ ID NOs: 1576-1578, SEQ ID NOs: 1600-1602, SEQ ID NOs: 1624-1636, SEQ ID NOs: 1648-1650, and SEQ ID NOs: 1672-1674. In some embodiments, the antibodies or antigen-binding fragments thereof described herein can also contain one, two, or three heavy chain variable region CDRs as shown in FIGS. 14, 42, and 43 under Chothia, IMGT, North, or Aho numbering scheme; and/or one, two, or three light chain variable region CDRs as shown in FIGS. 14, 42, and 43 under Chothia, IMGT, North, or Aho numbering scheme.

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence, and a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIGS. 14, 42, and 43.

In some embodiments, the antibody or an antigen-binding fragment described herein can contain a heavy chain variable domain containing one, two, or three of any one of the VH CDR1 shown in FIGS. 14, 42, and 43 with zero, one or two amino acid insertions, deletions, or substitutions; any one of the VH CDR2 shown in FIGS. 14, 42, and 43 with zero, one or two amino acid insertions, deletions, or substitutions; any one of the VH CDR3 shown in FIGS. 14, 42, and 43 with zero, one or two amino acid insertions, deletions, or substitutions. For example, the D2M002-001 antibody or an antigen-binding fragment described herein can contain a heavy chain variable domain containing one, two, or three of the CDRs of SEQ ID NO: 7 with zero, one or two amino acid insertions, deletions, or substitutions; SEQ ID NO: 8 with zero, one or two amino acid insertions, deletions, or substitutions; SEQ ID NO: 9 with zero, one or two amino acid insertions, deletions, or substitutions.

In some embodiments, the antibody or an antigen-binding fragment described herein can contain a light chain variable domain containing one, two, or three of any one of the VL CDR1 shown in FIGS. 14, 42, and 43 with zero, one or two amino acid insertions, deletions, or substitutions; any one of the VL CDR2 shown in FIGS. 14, 42, and 43 with zero, one or two amino acid insertions, deletions, or substitutions; any one of the VL CDR3 shown in FIGS. 14, 42, and 43 with zero, one or two amino acid insertions, deletions, or substitutions. In some embodiments, the D2M002-001 antibody or an antigen-binding fragment described herein can contain a light chain variable domain containing one, two, or three of the CDRs of SEQ ID NO: 10 with zero, one or two amino acid insertions, deletions, or substitutions; SEQ ID NO: 11 with zero, one or two amino acid insertions, deletions, or substitutions; SEQ ID NO: 12 with zero, one or two amino acid insertions, deletions, or substitutions.

The insertions, deletions, and substitutions can be within the CDR sequence, or at one or both terminal ends of the CDR sequence.

The disclosure also provides antibodies or antigen-binding fragments thereof that bind to LILRB1 and/or LILRB2. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 751, 753, 755, 757, 759, 761, 763, 765, 767, 769, 771, 773, 775, 777, 779, 781, 783, 785, 787, 789, 791, 793, 795, 797, 799, 841, 843, 845, 847, 849, 851, 853, 855, 857, 859, 861, 863, 865, 867, 869, 871, 873, 875, 877, 879, 881, 883, 885, 887, 889, 891, 893, 895, 897, 899, 901, 903, 905, 907, 909, 911, 913, 915, 917, 919, 921, 923, 925, 927, 929, 931, 933, 935, 937, 939, 941, 943, 945, 947, 949, 951, 953, 955, 957, 959, 961, 963, 965, 967, 969, 971, 973, 975, 977, 979, 985, 987, 993, 995, 997, 999, 1005, 1007, 1011, 1013, 1015, 1017, 1019, 1021, 1023, 1025, 1027, 1029, 1031, 1033, 1035, 1037, 1039, 1041, 1043, 1045, 1047, 1049, 1051, 1053, 1055, 1067, or 1068. In some embodiments, the selected VL sequence is 752, 754, 756, 758, 760, 762, 764, 766, 768, 770, 772, 774, 776, 778, 780, 782, 784, 786, 788, 790, 792, 794, 796, 798, 800, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 981, 982, 983, 984, 986, 988, 989, 990, 991, 992, 994, 996, 998, 1000, 1001, 1002, 1003, 1004, 1006, 1008, 1009, 1010, 1012, 1014, 1016, 1018, 1020, 1022, 1024, 1026, 1028, 1030, 1032, 1034, 1036, 1038, 1040, 1042, 1044, 1046, 1048, 1050, 1052, 1054, 1056, 1689, or 1690. In some embodiments, the selected VH sequence is SEQ ID NO: 751, and the selected VL sequence is SEQ ID NO: 752. In some embodiments, the selected VH sequence is SEQ ID NO: 753, and the selected VL sequence is SEQ ID NO: 754. In some embodiments, the selected VH sequence is SEQ ID NO: 755, and the selected VL sequence is SEQ ID NO: 756. In some embodiments, the selected VH sequence is SEQ ID NO: 757, and the selected VL sequence is SEQ ID NO: 758. In some embodiments, the selected VH sequence is SEQ ID NO: 759, and the selected VL sequence is SEQ ID NO: 760. In some embodiments, the selected VH sequence is SEQ ID NO: 761, and the selected VL sequence is SEQ ID NO: 762. In some embodiments, the selected VH sequence is SEQ ID NO: 763, and the selected VL sequence is SEQ ID NO: 764. In some embodiments, the selected VH sequence is SEQ ID NO: 765, and the selected VL sequence is SEQ ID NO: 766. In some embodiments, the selected VH sequence is SEQ ID NO: 767, and the selected VL sequence is SEQ ID NO: 768. In some embodiments, the selected VH sequence is SEQ ID NO: 769, and the selected VL sequence is SEQ ID NO: 770. In some embodiments, the selected VH sequence is SEQ ID NO: 771, and the selected VL sequence is SEQ ID NO: 772. In some embodiments, the selected VH sequence is SEQ ID NO: 773, and the selected VL sequence is SEQ ID NO: 774. In some embodiments, the selected VH sequence is SEQ ID NO: 775, and the selected VL sequence is SEQ ID NO: 776. In some embodiments, the selected VH sequence is SEQ ID NO: 777, and the selected VL sequence is SEQ ID NO: 778. In some embodiments, the selected VH sequence is SEQ ID NO: 779, and the selected VL sequence is SEQ ID NO: 780. In some embodiments, the selected VH sequence is SEQ ID NO: 781, and the selected VL sequence is SEQ ID NO: 782. In some embodiments, the selected VH sequence is SEQ ID NO: 783, and the selected VL sequence is SEQ ID NO: 784. In some embodiments, the selected VH sequence is SEQ ID NO: 785, and the selected VL sequence is SEQ ID NO: 786. In some embodiments, the selected VH sequence is SEQ ID NO: 787, and the selected VL sequence is SEQ ID NO: 788. In some embodiments, the selected VH sequence is SEQ ID NO: 789, and the selected VL sequence is SEQ ID NO: 790. In some embodiments, the selected VH sequence is SEQ ID NO: 791, and the selected VL sequence is SEQ ID NO: 792. In some embodiments, the selected VH sequence is SEQ ID NO: 793, and the selected VL sequence is SEQ ID NO: 794. In some embodiments, the selected VH sequence is SEQ ID NO: 795, and the selected VL sequence is SEQ ID NO: 796. In some embodiments, the selected VH sequence is SEQ ID NO: 797, and the selected VL sequence is SEQ ID NO: 798. In some embodiments, the selected VH sequence is SEQ ID NO: 799, and the selected VL sequence is SEQ ID NO: 800. In some embodiments, the selected VH sequence is SEQ ID NO: 841, and the selected VL sequence is SEQ ID NO: 842. In some embodiments, the selected VH sequence is SEQ ID NO: 843, and the selected VL sequence is SEQ ID NO: 844. In some embodiments, the selected VH sequence is SEQ ID NO: 845, and the selected VL sequence is SEQ ID NO: 846. In some embodiments, the selected VH sequence is SEQ ID NO: 847, and the selected VL sequence is SEQ ID NO: 848. In some embodiments, the selected VH sequence is SEQ ID NO: 849, and the selected VL sequence is SEQ ID NO: 850. In some embodiments, the selected VH sequence is SEQ ID NO: 851, and the selected VL sequence is SEQ ID NO: 852. In some embodiments, the selected VH sequence is SEQ ID NO: 853, and the selected VL sequence is SEQ ID NO: 854. In some embodiments, the selected VH sequence is SEQ ID NO: 855, and the selected VL sequence is SEQ ID NO: 856. In some embodiments, the selected VH sequence is SEQ ID NO: 857, and the selected VL sequence is SEQ ID NO: 858. In some embodiments, the selected VH sequence is SEQ ID NO: 859, and the selected VL sequence is SEQ ID NO: 860. In some embodiments, the selected VH sequence is SEQ ID NO: 861, and the selected VL sequence is SEQ ID NO: 862. In some embodiments, the selected VH sequence is SEQ ID NO: 863, and the selected VL sequence is SEQ ID NO: 864. In some embodiments, the selected VH sequence is SEQ ID NO: 865, and the selected VL sequence is SEQ ID NO: 866. In some embodiments, the selected VH sequence is SEQ ID NO: 867, and the selected VL sequence is SEQ ID NO: 868. In some embodiments, the selected VH sequence is SEQ ID NO: 869, and the selected VL sequence is SEQ ID NO: 870. In some embodiments, the selected VH sequence is SEQ ID NO: 871, and the selected VL sequence is SEQ ID NO: 872. In some embodiments, the selected VH sequence is SEQ ID NO: 873, and the selected VL sequence is SEQ ID NO: 874. In some embodiments, the selected VH sequence is SEQ ID NO: 875, and the selected VL sequence is SEQ ID NO: 876. In some embodiments, the selected VH sequence is SEQ ID NO: 877, and the selected VL sequence is SEQ ID NO: 878. In some embodiments, the selected VH sequence is SEQ ID NO: 879, and the selected VL sequence is SEQ ID NO: 880. In some embodiments, the selected VH sequence is SEQ ID NO: 881, and the selected VL sequence is SEQ ID NO: 882. In some embodiments, the selected VH sequence is SEQ ID NO: 883, and the selected VL sequence is SEQ ID NO: 884. In some embodiments, the selected VH sequence is SEQ ID NO: 885, and the selected VL sequence is SEQ ID NO: 886. In some embodiments, the selected VH sequence is SEQ ID NO: 887, and the selected VL sequence is SEQ ID NO: 888. In some embodiments, the selected VH sequence is SEQ ID NO: 889, and the selected VL sequence is SEQ ID NO: 890. In some embodiments, the selected VH sequence is SEQ ID NO: 891, and the selected VL sequence is SEQ ID NO: 892. In some embodiments, the selected VH sequence is SEQ ID NO: 893, and the selected VL sequence is SEQ ID NO: 894. In some embodiments, the selected VH sequence is SEQ ID NO: 895, and the selected VL sequence is SEQ ID NO: 896. In some embodiments, the selected VH sequence is SEQ ID NO: 897, and the selected VL sequence is SEQ ID NO: 898. In some embodiments, the selected VH sequence is SEQ ID NO: 899, and the selected VL sequence is SEQ ID NO: 900. In some embodiments, the selected VH sequence is SEQ ID NO: 901, and the selected VL sequence is SEQ ID NO: 902. In some embodiments, the selected VH sequence is SEQ ID NO: 903, and the selected VL sequence is SEQ ID NO: 904. In some embodiments, the selected VH sequence is SEQ ID NO: 905, and the selected VL sequence is SEQ ID NO: 906. In some embodiments, the selected VH sequence is SEQ ID NO: 907, and the selected VL sequence is SEQ ID NO: 908. In some embodiments, the selected VH sequence is SEQ ID NO: 909, and the selected VL sequence is SEQ ID NO: 910. In some embodiments, the selected VH sequence is SEQ ID NO: 911, and the selected VL sequence is SEQ ID NO: 912. In some embodiments, the selected VH sequence is SEQ ID NO: 913, and the selected VL sequence is SEQ ID NO: 914. In some embodiments, the selected VH sequence is SEQ ID NO: 915, and the selected VL sequence is SEQ ID NO: 916. In some embodiments, the selected VH sequence is SEQ ID NO: 917, and the selected VL sequence is SEQ ID NO: 918. In some embodiments, the selected VH sequence is SEQ ID NO: 919, and the selected VL sequence is SEQ ID NO: 920. In some embodiments, the selected VH sequence is SEQ ID NO: 921, and the selected VL sequence is SEQ ID NO: 922. In some embodiments, the selected VH sequence is SEQ ID NO: 923, and the selected VL sequence is SEQ ID NO: 924. In some embodiments, the selected VH sequence is SEQ ID NO: 925, and the selected VL sequence is SEQ ID NO: 926. In some embodiments, the selected VH sequence is SEQ ID NO: 927, and the selected VL sequence is SEQ ID NO: 928. In some embodiments, the selected VH sequence is SEQ ID NO: 929, and the selected VL sequence is SEQ ID NO: 930. In some embodiments, the selected VH sequence is SEQ ID NO: 931, and the selected VL sequence is SEQ ID NO: 932. In some embodiments, the selected VH sequence is SEQ ID NO: 933, and the selected VL sequence is SEQ ID NO: 934. In some embodiments, the selected VH sequence is SEQ ID NO: 935, and the selected VL sequence is SEQ ID NO: 936. In some embodiments, the selected VH sequence is SEQ ID NO: 937, and the selected VL sequence is SEQ ID NO: 938. In some embodiments, the selected VH sequence is SEQ ID NO: 939, and the selected VL sequence is SEQ ID NO: 940. In some embodiments, the selected VH sequence is SEQ ID NO: 941, and the selected VL sequence is SEQ ID NO: 942. In some embodiments, the selected VH sequence is SEQ ID NO: 943, and the selected VL sequence is SEQ ID NO: 944. In some embodiments, the selected VH sequence is SEQ ID NO: 945, and the selected VL sequence is SEQ ID NO: 946. In some embodiments, the selected VH sequence is SEQ ID NO: 947, and the selected VL sequence is SEQ ID NO: 948. In some embodiments, the selected VH sequence is SEQ ID NO: 949, and the selected VL sequence is SEQ ID NO: 950. In some embodiments, the selected VH sequence is SEQ ID NO: 951, and the selected VL sequence is SEQ ID NO: 952. In some embodiments, the selected VH sequence is SEQ ID NO: 953, and the selected VL sequence is SEQ ID NO: 954. In some embodiments, the selected VH sequence is SEQ ID NO: 955, and the selected VL sequence is SEQ ID NO: 956. In some embodiments, the selected VH sequence is SEQ ID NO: 957, and the selected VL sequence is SEQ ID NO: 958. In some embodiments, the selected VH sequence is SEQ ID NO: 959, and the selected VL sequence is SEQ ID NO: 960. In some embodiments, the selected VH sequence is SEQ ID NO: 961, and the selected VL sequence is SEQ ID NO: 962. In some embodiments, the selected VH sequence is SEQ ID NO: 963, and the selected VL sequence is SEQ ID NO: 964. In some embodiments, the selected VH sequence is SEQ ID NO: 965, and the selected VL sequence is SEQ ID NO: 966. In some embodiments, the selected VH sequence is SEQ ID NO: 967, and the selected VL sequence is SEQ ID NO: 968. In some embodiments, the selected VH sequence is SEQ ID NO: 969, and the selected VL sequence is SEQ ID NO: 970. In some embodiments, the selected VH sequence is SEQ ID NO: 971, and the selected VL sequence is SEQ ID NO: 972. In some embodiments, the selected VH sequence is SEQ ID NO: 973, and the selected VL sequence is SEQ ID NO: 974. In some embodiments, the selected VH sequence is SEQ ID NO: 975, and the selected VL sequence is SEQ ID NO: 976. In some embodiments, the selected VH sequence is SEQ ID NO: 977, and the selected VL sequence is SEQ ID NO: 978. In some embodiments, the selected VH sequence is SEQ ID NO: 979, and the selected VL sequence is SEQ ID NO: 980, 981, 982, 983, or 984. In some embodiments, the selected VH sequence is SEQ ID NO: 985, and the selected VL sequence is SEQ ID NO: 986. In some embodiments, the selected VH sequence is SEQ ID NO: 987, 1687, or 1688, and the selected VL sequence is SEQ ID NO: 988, 989, 990, 991, 992. In some embodiments, the selected VH sequence is SEQ ID NO: 987, and the selected VL sequence is SEQ ID NO: 988. In some embodiments, the selected VH sequence is SEQ ID NO: 1687, and the selected VL sequence is SEQ ID NO: 988. In some embodiments, the selected VH sequence is SEQ ID NO: 1688, and the selected VL sequence is SEQ ID NO: 988. In some embodiments, the selected VH sequence is SEQ ID NO: 987, and the selected VL sequence is SEQ ID NO: 989. In some embodiments, the selected VH sequence is SEQ ID NO: 987, and the selected VL sequence is SEQ ID NO: 990. In some embodiments, the selected VH sequence is SEQ ID NO: 987, and the selected VL sequence is SEQ ID NO: 991. In some embodiments, the selected VH sequence is SEQ ID NO: 987, and the selected VL sequence is SEQ ID NO: 992. In some embodiments, the selected VH sequence is SEQ ID NO: 993, and the selected VL sequence is SEQ ID NO: 994. In some embodiments, the selected VH sequence is SEQ ID NO: 995, and the selected VL sequence is SEQ ID NO: 996. In some embodiments, the selected VH sequence is SEQ ID NO: 997, and the selected VL sequence is SEQ ID NO: 998. In some embodiments, the selected VH sequence is SEQ ID NO: 999, and the selected VL sequence is SEQ ID NO: 1000, 1001, 1002, 1003, or 1004. In some embodiments, the selected VH sequence is SEQ ID NO: 1005, and the selected VL sequence is SEQ ID NO: 1006. In some embodiments, the selected VH sequence is SEQ ID NO: 1007, and the selected VL sequence is SEQ ID NO: 1008, 1009, or 1010. In some embodiments, the selected VH sequence is SEQ ID NO: 1011, and the selected VL sequence is SEQ ID NO: 1012. In some embodiments, the selected VH sequence is SEQ ID NO: 1013, and the selected VL sequence is SEQ ID NO: 1014. In some embodiments, the selected VH sequence is SEQ ID NO: 1015, and the selected VL sequence is SEQ ID NO: 1016. In some embodiments, the selected VH sequence is SEQ ID NO: 1017, and the selected VL sequence is SEQ ID NO: 1018. In some embodiments, the selected VH sequence is SEQ ID NO: 1019, and the selected VL sequence is SEQ ID NO: 1020. In some embodiments, the selected VH sequence is SEQ ID NO: 1021, and the selected VL sequence is SEQ ID NO: 1022.

In some embodiments, the selected VH sequence is SEQ ID NO: 1023, and the selected VL sequence is SEQ ID NO: 1024. In some embodiments, the selected VH sequence is SEQ ID NO: 1025, and the selected VL sequence is SEQ ID NO: 1026. In some embodiments, the selected VH sequence is SEQ ID NO: 1027, and the selected VL sequence is SEQ ID NO: 1028. In some embodiments, the selected VH sequence is SEQ ID NO: 1029, and the selected VL sequence is SEQ ID NO: 1030. In some embodiments, the selected VH sequence is SEQ ID NO: 1031, and the selected VL sequence is SEQ ID NO: 1032. In some embodiments, the selected VH sequence is SEQ ID NO: 1033, and the selected VL sequence is SEQ ID NO: 1034. In some embodiments, the selected VH sequence is SEQ ID NO: 1035, and the selected VL sequence is SEQ ID NO: 1036. In some embodiments, the selected VH sequence is SEQ ID NO: 1037, and the selected VL sequence is SEQ ID NO: 1038. In some embodiments, the selected VH sequence is SEQ ID NO: 1039, and the selected VL sequence is SEQ ID NO: 1040. In some embodiments, the selected VH sequence is SEQ ID NO: 1041, and the selected VL sequence is SEQ ID NO: 1042.

In some embodiments, the selected VH sequence is SEQ ID NO: 1043, and the selected VL sequence is SEQ ID NO: 1044, 1689, or 1690. In some embodiments, the selected VH sequence is SEQ ID NO: 1043, and the selected VL sequence is SEQ ID NO: 1044. In some embodiments, the selected VH sequence is SEQ ID NO: 1043, and the selected VL sequence is SEQ ID NO: 1689. In some embodiments, the selected VH sequence is SEQ ID NO: 1043, and the selected VL sequence is SEQ ID NO: 1690. In some embodiments, the selected VH sequence is SEQ ID NO: 1045, and the selected VL sequence is SEQ ID NO: 1046. In some embodiments, the selected VH sequence is SEQ ID NO: 1047, and the selected VL sequence is SEQ ID NO: 1048.

In some embodiments, the selected VH sequence is SEQ ID NO: 1049, and the selected VL sequence is SEQ ID NO: 1050. In some embodiments, the selected VH sequence is SEQ ID NO: 1051, and the selected VL sequence is SEQ ID NO: 1052. In some embodiments, the selected VH sequence is SEQ ID NO: 1053, and the selected VL sequence is SEQ ID NO: 1054. In some embodiments, the selected VH sequence is SEQ ID NO: 1055, and the selected VL sequence is SEQ ID NO: 1056.

The disclosure also provides antibodies or antigen-binding fragments thereof that bind to LILRB1 and/or LILRB2. The antibodies or antigen-binding fragments thereof contain a heavy chain comprising or consisting of an amino acid sequence that is at least 80%, 85%, 90%, or 95% identical to a selected heavy chain sequence, and a light chain comprising or consisting of an amino acid sequence that is at least 80%, 85%, 90%, or 95% identical to a selected light chain sequence. In some embodiments, the selected heavy chain is SEQ ID NO: 1691, and the selected light chain is SEQ ID NO: 1692. In some embodiments, the selected heavy chain is SEQ ID NO: 1693, and the selected light chain is SEQ ID NO: 1692. In some embodiments, the selected heavy chain is SEQ ID NO: 1694, and the selected light chain is SEQ ID NO: 1695.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

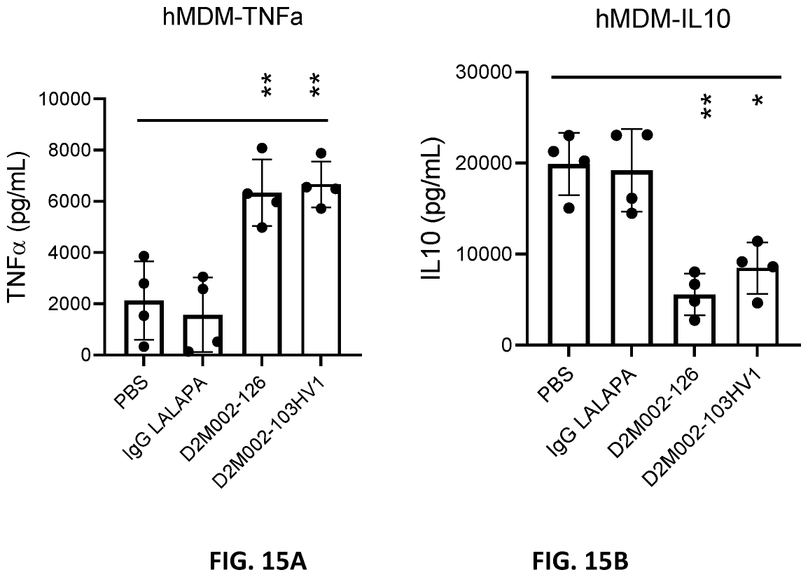

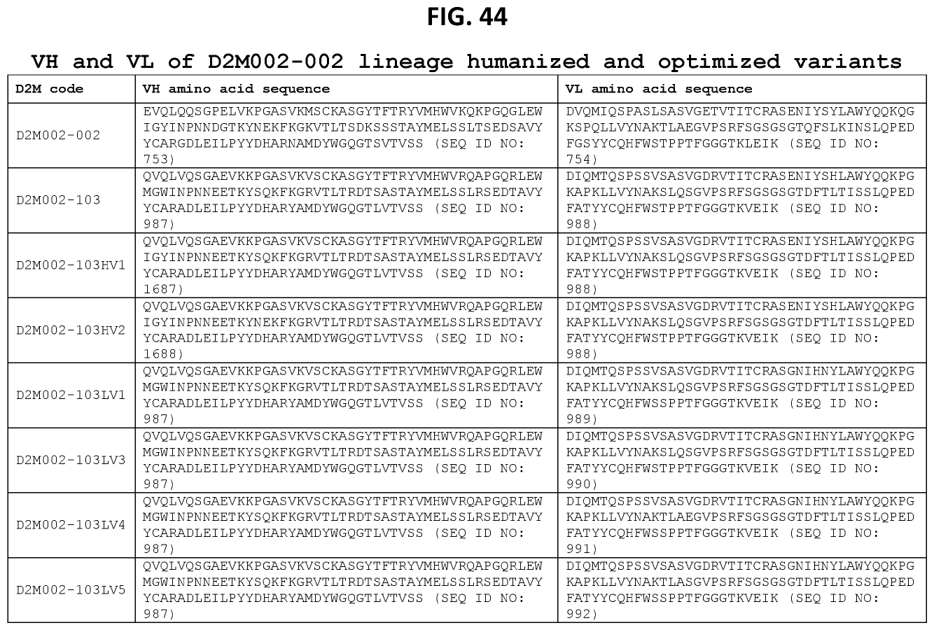

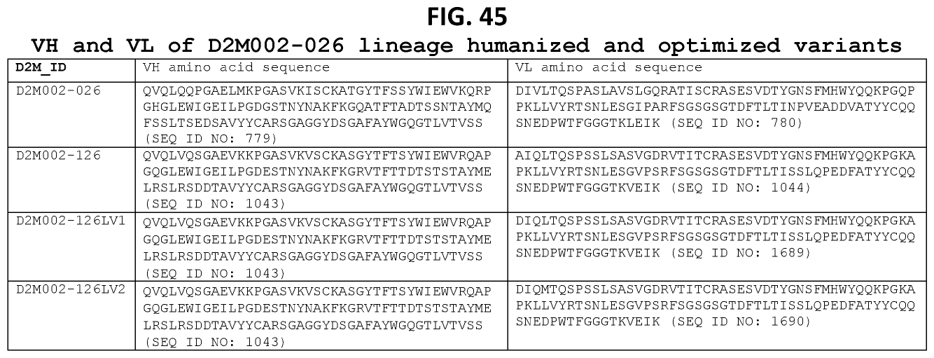

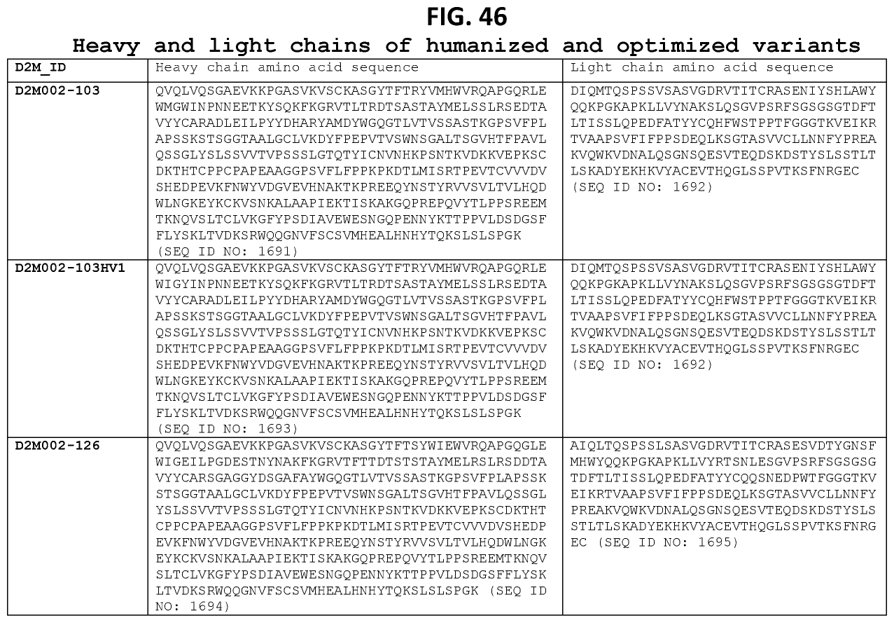

The disclosure also provides nucleic acid comprising a polynucleotide encoding a polypeptide comprising an immunoglobulin heavy chain or an immunoglobulin heavy chain. The immunoglobulin heavy chain or immunoglobulin light chain comprises CDRs (under Kabat, Chothia, IMGT, North, or Aho numbering) as shown in FIGS. 14, 42, and 43, or have sequences as shown in FIGS. 15, 44, and 45. When the polypeptides are paired with corresponding polypeptide (e.g., a corresponding heavy chain variable region or a corresponding light chain variable region), the paired polypeptides bind to LILRB1 (e.g., human LILRB1) and/or LILRB2 (e.g., human LILRB2).

The anti-LILRB1/2 antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to LILRB1 and/or LILRB2 will retain an ability to bind to LILRB1 and/or LILRB2. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

Single-chain Fv or (scFv) antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding.

The Fab fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

Diabodies are small antibody fragments with two antigen-binding sites, which fragments comprise a VH connected to a VL in the same polypeptide chain (VH and VL). By using a linker that is too short to allow pairing between the two domains on the same chain, the domains are forced to pair with the complementary domains of another chain and create two antigen-binding sites.

Linear antibodies comprise a pair of tandem Fd segments (VH-CH1-VH-CH1) which, together with complementary light chain polypeptides, form a pair of antigen binding regions. Linear antibodies can be bispecific or monospecific.

Antibodies and antibody fragments of the present disclosure can be modified in the Fc region to provide desired effector functions or serum half-life.

Multimerization of antibodies may be accomplished through natural aggregation of antibodies or through chemical or recombinant linking techniques known in the art. For example, some percentage of purified antibody preparations (e.g., purified IgG₁ molecules) spontaneously form protein aggregates containing antibody homodimers and other higher-order antibody multimers.

Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) can be used to form antibody multimers. An exemplary protocol for the formation of antibody homodimers is described in Ghetie et al. (Proc. Natl. Acad. Sci. U.S.A. 94: 7509-7514, 1997). Antibody homodimers can be converted to Fab′₂ homodimers through digestion with pepsin. Another way to form antibody homodimers is through the use of the autophilic T15 peptide described in Zhao et al. (J. Immunol. 25:396-404, 2002).

In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Bi-specific antibodies include cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art and are disclosed in U.S. Pat. No. 4,676,980, which is incorporated herein by reference in its entirety.

Methods for generating bi-specific antibodies from antibody fragments are also known in the art. For example, bi-specific antibodies can be prepared using chemical linkage. Brennan et al. (Science 229:81, 1985) describes a procedure where intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′ TNB derivatives is then reconverted to the Fab′ thiol by reduction with mercaptoethylamine, and is mixed with an equimolar amount of another Fab′ TNB derivative to form the bi-specific antibody.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibodies or antigen-binding fragments described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

### Antibodies and Antigen Binding Fragments

The present disclosure provides anti-LILRB1/2 antibodies and antigen-binding fragments thereof. In general, antibodies (also called immunoglobulins) are made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and two identical copies of a heavy chain. The heavy chains, which each contain one variable domain (or variable region, V_{H}) and multiple constant domains (or constant regions), bind to one another via disulfide bonding within their constant domains to form the “stem” of the antibody. The light chains, which each contain one variable domain (or variable region, V_{L}) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132: 211-250; Martin et al., Methods Enzymol. 203:121-53 (1991); Morea et al., Biophys Chem. 68(1-3):9-16 (October 1997); Morea et al., J Mol Biol. 275(2):269-94 (January 1998); Chothia et al., Nature 342(6252):877-83 (December 1989); Ponomarenko and Bourne, BMC Structural Biology 7:64 (2007); each of which is incorporated herein by reference in its entirety. Unless specifically indicated in the present disclosure, Kabat numbering is used in the present disclosure as a default.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)₂, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

In some embodiments, the scFV has one heavy chain variable domain, and one light chain variable domain.

### Antibody Characteristics

The antibodies or antigen-binding fragments thereof described herein can block the binding between LILRB1 and/or LILRB2 with MHC Class I molecules (e.g., HLA-G).

LILRB1 and LILRB2 are expressed on immune cells where it binds to MHC class I molecules on antigen-presenting cells and transduces a negative signal that inhibits stimulation of an immune response. In some embodiments, by binding to LILRB1 and/or LILRB2, the antibodies described herein can block the negative signal that inhibits stimulation of an immune response, thereby upregulating the immune response. Thus, in some embodiments, the antibodies or antigen-binding fragments thereof as described herein are agonists of LILRB1 and/or LILRB2. In some embodiments, the antibodies or antigen-binding fragments thereof are antagonists of LILRB1 and/or LILRB2.

In some embodiments, the antibodies or antigen-binding fragments thereof as described herein can increase immune response, activity or number of T cells (e.g., CD8+ and/or CD4+ cells), or cytokine production (e.g., TNFα) by at least 10%, 20%, 30%, 40%, 50%, 60%, 70%, 80%, 90%, 100%, 2 folds, 3 folds, 5 folds, 10 folds, or 20 folds. In some embodiments, the antibodies or antigen-binding fragments thereof as described herein can decrease the activity or number of Treg, or cytokine production (e.g., IL10) by at least 10%, 20%, 30%, 40%, 50%, 60%, 70%, 80%, 90%, 100%, 2 folds, 3 folds, 5 folds, 10 folds, or 20 folds.

In some implementations, the antibody (or antigen-binding fragments thereof) specifically binds to LILRB1 (e.g., human LILRB1, monkey LILRB1, mouse LILRB1, and/or chimeric LILRB1) or LILRB2 (e.g., human LILRB2, monkey LILRB2, mouse LILRB2, and/or chimeric LILRB2) with a dissociation rate (koff) of less than 0.1 s⁻¹, less than 0.01 s⁻¹, less than 0.001 s⁻¹, less than 0.0001 s⁻¹, or less than 0.0001 s⁻¹. In some embodiments, the dissociation rate (koff) is greater than 0.01 s⁻¹, greater than 0.001 s⁻¹, greater than 0.0001 s⁻¹, greater than 0.0001 s₋₁, or greater than 0.00001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (KD=koff/kon). In some embodiments, KD is less than 1×10⁻⁶ M, less than 1×10⁻⁷ M, less than 1×10⁻⁸ M, less than 1×10⁻⁹ M, or less than 1×10⁻¹⁰ M. In some embodiments, the KD is less than 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷ M, greater than 1×10⁻⁸ M, greater than 1×10⁻⁹ M, greater than 1×10⁻¹⁰ M, greater than 1×10⁻⁴ M, or greater than 1×10⁻¹² M.

General techniques for measuring the affinity of an antibody for an antigen include, e.g., ELISA, RIA, and surface plasmon resonance (SPR). In some embodiments, the measurement is conducted using Carterra® SPR imaging system or Gator Prime BLI system. In some embodiments, the antibody binds to human LILRB1, monkey LILRB1 (e.g., cynomolgus LILRB1), and/or chimeric LILRB1. In some embodiments, the antibody binds to human LILRB2, monkey LILRB2 (e.g., cynomolgus LILRB2), and/or chimeric LILRB2.

In some embodiments, the anti-LILRB1/2 antibodies or antigen-binding fragments thereof described herein can bind to human LILRB1 and/or LILRB2 with an EC50 value of less than 10 μg/mL, less than 9 μg/mL, less than 8 μg/mL, less than 7 μg/mL, less than 6 μg/mL, less than 5 μg/mL, less than 4 μg/mL, less than 3 μg/mL, less than 2 μg/mL, less than 1 μg/mL, less than 0.9 μg/mL, less than 0.8 μg/mL, less than 0.7 μg/mL, less than 0.6 μg/mL, less than 0.5 μg/mL, less than 0.4 μg/mL, less than 0.3 μg/mL, less than 0.2 μg/mL, or less than 0.1 μg/mL. In some embodiments, the antibodies described herein can block the LILRB1/HLA-G interaction and/or LILRB2/HLA-G interaction with an IC50 value of less than less than 2 μg/mL, less than 1 μg/mL, less than 0.9 μg/mL, less than 0.8 μg/mL, less than 0.7 μg/mL, less than 0.6 μg/mL, less than 0.5 μg/mL, less than 0.4 μg/mL, less than 0.3 μg/mL, less than 0.2 μg/mL, less than 0.1 μg/mL, less than 0.05 μg/mL, less than 0.04 μg/mL, less than 0.03 μg/mL, less than 0.02 μg/mL, or less than 0.01 μg/mL.

In some embodiments, the anti-LILRB1/2 antibodies or antigen-binding fragments thereof described herein can bind to monkey LILRB1 or LILRB2 with an EC50 value of less than 10 μg/mL, less than 9 μg/mL, less than 8 μg/mL, less than 7 μg/mL, less than 6 μg/mL, less than 5 μg/mL, less than 4 μg/mL, less than 3 μg/mL, less than 2 μg/mL, or less than 1 μg/mL. In some embodiments, the antibodies described herein can bind to monkey LILRB1 or LILRB2 with a KD value of less than 1×10⁻⁶ M, less than 1×10⁻⁷ M, less than 1×10⁻⁸ M, less than 1×10⁻⁹ M, or less than 1×10⁻¹⁰ M. In some embodiments, the antibodies described herein do not bind to monkey LILRB1 or LILRB2.

In some embodiments, the anti-LILRB1/2 antibodies or antigen-binding fragments thereof described herein can bind to any one of LILRBA/B family members including LILRA family members (e.g., LILRA1, LILRA2, LILRA3, LILRA4, LILRA5, and/or LILRA6), and LILRB family members (e.g., LILRB1, LILRB2, LILRB3, LILRB4, and/or LILRB5). In some embodiments, the antibodies described herein do not bind to any one of LILRBA/B family members including LILRA family members (e.g., LILRA1, LILRA2, LILRA3, LILRA4, LILRA5, and/or LILRA6), and LILRB family members (e.g., LILRB1, LILRB2, LILRB3, LILRB4, and/or LILRB5). In some embodiments, the antibodies described herein (e.g., any of the D2M002-002 and/or D2M002-026 lineage antibodies or variants described herein) can bind to LILRB1 (e.g., human LILRB1), LILRB2 (e.g., human LILRB2), and/or LILRA1 (e.g., human LILRA1) with an EC50 value of less than 10 μg/mL, less than 10 μg/mL, less than 9 μg/mL, less than 8 μg/mL, less than 7 μg/mL, less than 6 μg/mL, less than 5 μg/mL, less than 4 μg/mL, less than 3 μg/mL, less than 2 μg/mL, less than 1 μg/mL, less than 0.5 μg/mL, or less than 0.1 μg/mL. In some embodiments, the antibodies described herein (e.g., any of the D2M002-002 lineage antibodies or variants described herein) can bind to LILRB3 (e.g., human LILRB3), LILRA6 (e.g., human LILRA6), and/or LILRB2 (e.g., monkey LILRB2) with an EC50 value of less than 10 μg/mL, less than 10 μg/mL, less than 9 μg/mL, less than 8 μg/mL, less than 7 μg/mL, less than 6 μg/mL, less than 5 μg/mL, less than 4 μg/mL, less than 3 μg/mL, less than 2 μg/mL, less than 1 μg/mL, less than 0.5 μg/mL, or less than 0.1 μg/mL. In some embodiments, the corresponding EC50 value is less than 50 nM, less than 40 nM, less than 30 nM, less than 20 nM, or less than 10 nM.

In some embodiments, the anti-LILRB1/2 antibodies or antigen-binding fragments thereof described herein (e.g., any of the D2M002-026 lineage antibodies or variants described herein) can block LILRB2 binding to its ligands (e.g., SEMA4A, MAG, CD1C, CD1D, ANGPTL2 and/or ANGPTL5).

In some embodiments, thermal stabilities are determined. The antibodies or antigen-binding fragments thereof as described herein can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. As IgG can be described as a multi-domain protein, the melting curve sometimes shows two transitions, with a first denaturation temperature, Tm D1, and a second denaturation temperature Tm D2. The presence of these two peaks often indicate the denaturation of the Fc domains (Tm D1) and Fab domains (Tm D2), respectively. When there are two peaks, Tm usually refers to Tm D2.

Thus, in some embodiments, the antibodies or antigen-binding fragments thereof as described herein has a Tm D1 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, the antibodies or antigen binding fragments as described herein has a Tm D2 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, Tm, Tm D1, Tm D2 are less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the anti-LILRB1/2 antibodies or antigen-binding fragments thereof described herein (e.g., any of the D2M002-026 lineage antibodies or variants described herein) can be mixed with fresh human plasma, and stored at about 30-40° C. (e.g., about 37° C.) for at least 1 week, 2 weeks, 3 weeks, 4 weeks, or 5 weeks, without showing significant loss of activities (e.g., binding to LILRB1 and/or LILRB2).

In some embodiments, the anti-LILRB1/2 antibodies or antigen-binding fragments thereof described herein (e.g., any of the D2M002-026 lineage antibodies or variants described herein) can maintain an in vivo concentration of at least 95%, at least 90%, at least 85%, at least 80%, at least 75%, at least 70%, at least 65%, at least 60%, at least 65%, or at least 50% as compared to the original in vivo concentration (determined immediately after injection), after about 1 day, 2 days, 3 days, 4 days, 5 days, 6 days, 7 days, 8 days, 9 days, or 10 days post injection into an animal (e.g., mouse).

In some embodiments, the antibody has a tumor growth inhibition percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

$$TGI ( \% ) = [ 1 - ( Ti - T 0 ) / ( Vi - V 0 ) ] \times 100$$

Ti is the average tumor volume in the treatment group on day i. T0 is the average tumor volume in the treatment group on day zero. Vi is the average tumor volume in the control group on day i. V0 is the average tumor volume in the control group on day zero.

In some embodiments, the anti-LILRB1/LILRB2 antibody or antigen-binding fragment thereof described herein can be mixed with macrophages (e.g., hMDM) for treating cancer. In some embodiments, the mixture is injected into subjects (e.g., immuno-compromised animals), and tumor volume is monitored over time. In some embodiments, the mixture is administered by subcutaneous injection, or intratumor injection. In some embodiments, the antibodies can reprogram the macrophages, and significantly inhibit tumor growth, e.g., with a TGI that is at least 10%, at least 20%, at least 30%, at least 40%, or at least 50% higher than that of an isotype control.

In some embodiments, the anti-LILRB1/2 anti-LILRB1/LILRB2 antibody or antigen-binding fragment thereof described herein can be administered to subjects (e.g., immuno-compromised animals) that are pre-injected with human PBMC. In some embodiments, the antibodies can significantly inhibit tumor growth in the PBMC-reconstituted animal model, e.g., with a TGI that is at least 10%, at least 20%, at least 30%, at least 40%, or at least 50% higher than that of an isotype control.

In some embodiments, the anti-LILRB1/2 antibody or antigen-binding fragment thereof described herein has a functional Fc. In some embodiments, the Fc is from human IgG1, human IgG2, human IgG3, or human IgG4. In some embodiments, effector function of a functional Fc is antibody-dependent cell-mediated cytotoxicity (ADCC). In some embodiments, effector function of a functional Fc is phagocytosis (e.g., antibody-dependent cellular phagocytosis, or ADCP). In some embodiments, effector function of a functional Fc is ADCC and phagocytosis. In some embodiments, the antibody or antigen-binding fragment thereof as described herein have an Fc region without effector function. In some embodiments, the Fc is a human IgG4 Fc. In some embodiments, the Fc is a human IgG1 Fc. In some embodiments, the Fc does not have a functional Fc region. For example, the Fc region has LALA mutations (L234A and L235A mutations in EU numbering), LALA-PA mutations (L234A, L235A, and P329A), or LALA-PG mutations (L234A, L235A, P329G mutations in EU numbering). Details of the LALA-PG mutations can be found, e.g., in PCT Application Publication No. WO2012/130831, U.S. Pat. Nos. 8,969,526 and 10,093,714; each of which is incorporated herein by reference in its entirety. Details of the LALA-PA mutations can be found, e.g., U.S. Patent Application Publication No. US20210087271A1, which is incorporated herein by reference in its entirety.

Additional references regarding the LALA mutations can be found, e.g., in Wines, B. D., et al. “The IgG Fc contains distinct Fc receptor (FcR) binding sites: the leukocyte receptors FcγRI and FcγRIIa bind to a region in the Fc distinct from that recognized by neonatal FcR and protein A.” The Journal of Immunology 164.10 (2000): 5313-5318; Chappel, M. S., et al. “Identification of the Fc gamma receptor class I binding site in human IgG through the use of recombinant IgG1/IgG2 hybrid and point-mutated antibodies.” Proceedings of the National Academy of Sciences 88.20 (1991): 9036-9040; Wilkinson, I., et al. “Fc-engineered antibodies with immune effector functions completely abolished.” Plos one 16.12 (2021): e0260954; and Lund, J., et al. “Human Fc gamma RI and Fc gamma RII interact with distinct but overlapping sites on human IgG.” Journal of immunology (Baltimore, Md.: 1950) 147.8 (1991): 2657-2662; each of which is incorporated herein by reference in its entirety. Additional references regarding the P329A mutation can be found, e.g., in U.S. Pat. No. 6,528,624; Sondermann, P., et al. “The 3.2-Å crystal structure of the human IgG1 Fc fragment-FcγRIII complex.” Nature 406.6793 (2000): 267-273; Idusogie, E. E., et al. “Mapping of the C1q binding site on rituxan, a chimeric antibody with a human IgG1 Fc.” The Journal of Immunology 164.8 (2000): 4178-4184; and Shields, R. L., et al. “High resolution mapping of the binding site on human IgG1 for FcγRI, FcγRII, FcγRIII, and FcRn and design of IgG1 variants with improved binding to the FcγR.” Journal of Biological Chemistry 276.9 (2001): 6591-6604; each of which is incorporated herein by reference in its entirety. In some embodiments, the Fc region includes an amino acid sequence that is at least 80%, at least 85%, at least 90%, at least 95%, or 100% identical to SEQ ID NO: 801, 802, 803, 804, or 805. For example, the antibodies or antigen binding fragments are Fab, Fab′, F(ab′)₂, and Fv fragments.

In some embodiments, the anti-LILRB1/2 antibodies or antigen-binding fragments thereof described herein can only bind to LILRB1 (e.g., #4), therefore can also be named as an anti-LILRB1 antibody. In some embodiments, the anti-LILRB1/2 antibodies described herein can only bind to LILRB2 (e.g., #44), therefore can also be named as an anti-LILRB2 antibody.

In some embodiments, the blocking efficiency (e.g., between LILRB1 or LILRB2 with MHC Class I molecules) as described herein is greater than 50%, 55%, 60%, 65%, 70%, 75%, 80%, 85%, 90%, or 95%. In some embodiments, the binding EC50 concentration of the anti-LILRB1/2 antibodies described herein is less than 5 μg/mL, 4 μg/mL, 3 μg/mL, 2 μg/mL, 1 μg/mL, 0.9 μg/mL, 0.8 μg/mL, 0.7 μg/mL, 0.6 μg/mL, 0.5 μg/mL, 0.4 μg/mL, 0.3 μg/mL, 0.2 μg/mL, 0.1 μg/mL, or 0.05 μg/mL.

In some embodiments, the anti-LILRB1/2 antibodies or antigen-binding fragments thereof described herein can release the suppressive signal to activated T cells by blocking LILRB1/HLA-G interaction or LILRB2/HLA-G interaction. As a result, the activation level of T cells is increased by at least 10%, at least 20%, at least 30%, at least 40%, at least 50%, at least 60%, at least 70%, at least 80%, at least 90%, or at least 100% as compared to that of negative control cells (e.g., T cells without treatment of the anti-LILRB1/2 antibodies). In some embodiments, the T cells (e.g., Jurkat cells) are co-cultured with tumor cells (e.g., CIR cells) expressing MHC class I molecules (e.g., HLA-G) during antibody treatment. In some embodiments, the EC50 value of the anti-LILRB1/2 antibodies described herein to restore the activation of activated T cells (e.g., by blocking LILRB1/HLA-G interaction or LILRB2/HLA-G interaction) is less than 3 μg/mL, less than 2 μg/mL, less than 1 μg/mL, less than 0.9 μg/mL, less than 0.8 μg/mL, less than 0.7 μg/mL, less than 0.6 μg/mL, less than 0.5 μg/mL, less than 0.4 μg/mL, less than 0.3 μg/mL, less than 0.2 μg/mL, less than 0.1 μg/mL, less than 0.05 μg/mL, less than 0.04 μg/mL, less than 0.03 μg/mL, less than 0.02 μg/mL, or less than 0.01 μg/mL.

In some embodiments, the immune cells (T cells or PBMCs) are activated by LPS or anti-CD3 antibodies.

In some embodiments, the anti-LILRB1/2 antibodies or antigen-binding fragments thereof described herein can enhance the production of pro-inflammatory cytokines (e.g., TNFα) of immune cells (e.g., PBMCs) by at least 10%, at least 20%, at least 30%, at least 40%, at least 50%, at least 60%, at least 70%, at least 80%, at least 90%, or at least 100% as compared to negative control cells (e.g., cells without treatment or treated with PBS or isotype control). In some embodiments, the anti-LILRB1/2 antibodies described herein can reduce the production of anti-inflammatory cytokines (e.g., IL10) of immune cells (e.g., PBMCs) to less than 90%, less than 80%, less than 70%, less than 60%, less than 50%, less than 40%, less than 30%, less than 20%, or less than 10% as compared to negative control cells (e.g., cells without treatment or treated with PBS or isotype control). In some embodiments, the anti-LILRB1/2 antibodies described herein can enhance the production of pro-inflammatory cytokines (e.g., TNFα) of immune cells (e.g., PBMCs) with an EC50 value that is less than 90%, less than 80%, less than 70%, less than 60%, less than 50%, less than 40%, less than 30%, less than 20%, or less than 10% as compared to a reference antibody (e.g., Hz73D1.v1). In some embodiments, the anti-LILRB1/2 antibodies described herein can reduce the production of anti-inflammatory cytokines (e.g., IL10) of immune cells (e.g., PBMCs) with an IC50 value that is less than 90%, less than 80%, less than 70%, less than 60%, less than 50%, less than 40%, less than 30%, less than 20%, or less than 10% as compared to a reference antibody (e.g., Hz73D1.v1). In some embodiments, the immune cells described herein are activated by a low dose of LPS (e.g., less than about 100 ng/mL, 80 ng/mL, 50 ng/mL, 30 ng/mL, or 10 ng/mL).

In some embodiments, the anti-LILRB1/2 antibodies or antigen-binding fragments thereof described herein can polarize human monocytes to pro-inflammatory macrophages. For example, treatment of the anti-LILRB1/2 antibodies can decrease the expression of myeloid cell markers (e.g., CD14) or anti-inflammatory markers (e.g., CD163 and/or CD206) to less than 90%, less than 80%, less than 70%, less than 60%, less than 50%, less than 40%, less than 30%, less than 20%, or less than 10% as compared to that of negative control cells (e.g., cells treated with medium, PBS, or isotype control). In some embodiments, treatment of the anti-LILRB1/2 antibodies can increase the expression of myeloid cell markers (e.g., CD11B) by at least 10%, at least 20%, at least 30%, at least 40%, at least 50%, at least 60%, at least 70%, at least 80%, at least 90%, or at least 100% as compared to that of negative control cells (e.g., cells treated with medium, PBS, or isotype control). In some embodiments, treatment of the anti-LILRB1/2 antibodies can decrease the expression of immunosuppressive markers or inhibitory immune checkpoint molecules (e.g., PD-L1) to less than 90%, less than 80%, less than 70%, less than 60%, less than 50%, less than 40%, less than 30%, less than 20%, or less than 10% as compared to negative control cells (e.g., cells treated with PBS or isotype control).

In some embodiments, treatment of the anti-LILRB1/2 antibodies or antigen-binding fragments thereof described herein to LPS-treated microphages (e.g., differentiated microphages) can decrease the expression of inhibitory immune checkpoint molecules (e.g., PD-L1) and/or anti-inflammatory cytokines (e.g., IL10) to less than 90%, less than 80%, less than 70%, less than 60%, less than 50%, less than 40%, less than 30%, less than 20%, or less than 10% as compared to negative control cells (e.g., cells treated with medium, PBS, or isotype control). In some embodiments, treatment of anti-LILRB1/2 antibodies described herein to LPS-treated microphages (e.g., differentiated microphages) can increase the expression of pro-inflammatory cytokines (e.g., TNFα) by at least 10%, at least 20%, at least 30%, at least 40%, at least 50%, at least 60%, at least 70%, at least 80%, at least 90%, at least 100%, at least 150%, at least 200%, or at least 300% as compared to that of negative control cells (e.g., cells treated with medium, PBS, or isotype control). In some embodiments, the macrophages are treated with LPS prior to or after treatment of the anti-LILRB1/2 antibodies. In some embodiments, the LPS is about 100 ng/mL.

In some embodiments, the anti-LILRB1/2 antibodies or antigen-binding fragments thereof described herein can reprogram tumor-associated macrophages (TAMs). For example, treatment of the anti-LILRB1/2 antibodies can decrease the expression of CD14, anti-inflammatory markers (e.g., CD163 and/or CD206), or inhibitory immune checkpoint molecules (e.g., PD-L1) to less than 90%, less than 80%, less than 70%, less than 60%, less than 50%, less than 40%, less than 30%, less than 20%, or less than 10% as compared to that of negative control cells (e.g., cells treated with medium, PBS, or isotype control). In some embodiments, treatment of the anti-LILRB1/2 antibodies can decrease the proportion of tumor cells when co-cultured with TAMs to less than 90%, less than 80%, less than 70%, less than 60%, less than 50%, less than 40%, less than 30%, less than 20%, or less than 10% as compared to negative control cells (e.g., cells treated with medium, PBS, or isotype control).

In some embodiments, treatment of the anti-LILRB1/2 antibodies or antigen-binding fragments thereof described herein to LPS-treated TAMs can decrease the expression of inhibitory immune checkpoint molecules (e.g., PD-L1) and/or anti-inflammatory cytokines (e.g., IL10) to less than 90%, less than 80%, less than 70%, less than 60%, less than 50%, less than 40%, less than 30%, less than 20%, or less than 10% as compared to negative control cells (e.g., cells treated with medium, PBS, or isotype control). In some embodiments, treatment of anti-LILRB1/2 antibodies described herein to LPS-treated TAMs can increase the expression of pro-inflammatory cytokines (e.g., TNFα) by at least 10%, at least 20%, at least 30%, at least 40%, at least 50%, at least 60%, at least 70%, at least 80%, at least 90%, at least 100%, or at least 2-fold, 3-fold, 4-fold, 5-fold, 6-fold, 7-fold, 8-fold, 9-fold, 10-fold, 20-fold, 50-fold, 100-fold, 200-fold, 500-fold, or 1000-fold as compared to that of negative control cells (e.g., cells treated with medium, PBS, or isotype control). In some embodiments, the TAMs are treated with LPS prior to or after treatment of the anti-LILRB1/2 antibodies. In some embodiments, the LPS is 10⁻³⁰⁰ ng/mL (e.g., about 100 ng/mL).

In some embodiments, the anti-LILRB1/2 antibodies or antigen-binding fragments thereof described herein can inhibit the myeloid-dependent suppression of T cells activation. For example, treatment of the anti-LILRB1/2 antibodies described herein can increase IFN-γ secretion in mixed lymphocyte reactions (MVLR) by at least 10%, at least 20%, at least 30%, at least 40%, at least 50%, at least 60%, at least 70%, at least 80%, at least 90%, or at least 100% as compared to that when the anti-LILRB1/2 antibodies are not present.

In some embodiments, the anti-LILRB1/2 antibodies or antigen-binding fragments thereof described herein can increase phagocytosis of tumor cells (e.g., tumor cells expressing MHC Class I molecules) by macrophages by at least 10%, at least 20%, at least 30%, at least 40%, at least 50%, at least 60%, at least 70%, at least 80%, at least 90%, or at least 100% as compared to negative control cells (e.g., cells treated with PBS or isotype control). In some embodiments, the anti-LILRB1/2 antibodies described herein can increase antibody-dependent cellular phagocytosis (ADCP) of tumor cells by macrophages by at least 10%, at least 20%, at least 30%, at least 40%, at least 50%, at least 60%, at least 70%, at least 80%, at least 90%, or at least 100% as compared to negative control cells (e.g., cells treated with PBS or isotype control).

In some embodiments, the anti-LILRB1/2 antibodies or antigen-binding fragments thereof described herein can enhance antibody-dependent cellular phagocytosis (ADCP), in combination with an anti-CD47 antibody. In some embodiments, the anti-CD47 antibody has a concentration of about 0.01 μg/mL to about 10 μg/mL (e.g., about 0.1-1 μg/mL). In some embodiments, the combination of the anti-LILRB1/2 antibodies described herein and the anti-CD47 antibody can increase ADCP of tumor cells (e.g., melanoma) by macrophages by at least 10%, at least 20%, at least 30%, at least 40%, at least 50%, at least 60%, at least 70%, at least 80%, at least 90%, or at least 100% as compared to a reference antibody (e.g., 15G8, 1C1, 1E1(QE), or J-10.H1) or negative control cells (e.g., cells treated with PBS or isotype control).

In some embodiments, the anti-LILRB1/2 antibodies or antigen-binding fragments thereof described herein can enhance the cytotoxicity of human NK cells against tumor cells. In some embodiments, the NK cells express LILRB1. In some embodiments, the anti-LILRB1/2 antibodies described herein (e.g., any of the D2M002-026 lineage antibodies or variants described herein) can increase the cytotoxicity of the NK cells by at least at least 10%, at least 20%, at least 30%, at least 40%, at least 50%, at least 60%, at least 70%, at least 80%, at least 90%, or at least 100% as compared to a reference antibody (e.g., Hz73D1.v1) or negative control cells (e.g., cells treated with PBS or isotype control).

In some embodiments, the anti-LILRB1/2 antibodies or antigen-binding fragments thereof described herein can enhance the cytotoxicity of human CD8+ T cells against tumor cells. In some embodiments, the CD8+ T cells express LILRB1. In some embodiments, the anti-LILRB1/2 antibodies described herein (e.g., any of the D2M002-026 lineage antibodies or variants described herein) can increase the cytotoxicity of the NK cells by at least at least 10%, at least 20%, at least 30%, at least 40%, at least 50%, at least 60%, at least 70%, at least 80%, at least 90%, at least 100%, at least 150%, at least 200%, at least 300%, at least 400%, or at least 500% as compared to a reference antibody (e.g., 3C1, 15G8, or Hz73D1.v1) or negative control cells (e.g., cells treated with PBS or isotype control). In some embodiments, the anti-LILRB1/2 antibodies or antigen-binding fragments thereof described herein share the same binding epitopes with the reference antibodies (e.g., any of the reference antibodies described herein). In some embodiments, the anti-LILRB1/2 antibodies described herein have different binding epitopes with the reference antibodies (e.g., any of the reference antibodies described herein). In some embodiments, the anti-LILRB1/2 antibodies described herein have overlapped binding epitopes with the reference antibodies (e.g., any of the reference antibodies described herein).

Anti-LILRB1 antibody 3C1 comprises a heavy chain with amino acid sequence set forth in SEQ ID NO: 840, and a light chain with amino acid sequence set forth in SEQ ID NO: 839. Details of 3C1 can be found, e.g., in PCT Publication No. WO2020023268A1, which is incorporated herein by reference in its entirety. Anti-LILRB2 antibody 1E1(QE) comprises a heavy chain with amino acid sequence set forth in SEQ ID NO: 838, and a light chain with amino acid sequence set forth in SEQ ID NO: 837. Details of 1E1(QE) can be found, e.g., in PCT Publication No. WO2018187518A1 and U.S. Patent Application No. US20180298096A1; each of which is incorporated herein by reference in its entirety. In some embodiments, 1E1(QE) described herein is the 1E1 or Q1E clone disclosed in WO2018187518A1 or US20180298096A1.

Anti-LILRB1/2 antibody Hz73D1.v1 (or NGM707) comprises a heavy chain with amino acid sequence set forth in SEQ ID NO: 1062, and a light chain with amino acid sequence set forth in SEQ ID NO: 1061. Anti-LILRB2 antibody J-19.H1 (or JTX-8064) comprises a heavy chain with amino acid sequence set forth in SEQ ID NO: 1058, and a light chain with amino acid sequence set forth in SEQ ID NO: 1057. Anti-LILRB1 antibody 15G8 (or BND-22) comprises a heavy chain with amino acid sequence set forth in SEQ ID NO: 1060, and a light chain with amino acid sequence set forth in SEQ ID NO: 1059.

### Methods of Making Anti-LILRB1/2 Antibodies

An isolated fragment of human LILRB1 and/or LILRB2 can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of LILRB1 and/or LILRB2, and encompasses an epitope of the protein such that an antibody raised against the peptide forms a specific immune complex with the protein. As described above, the full length sequence of human LILRB1 is known in the art (SEQ ID NO: 813), and the full length sequence of human LILRB2 is known in the art (SEQ ID NO: 814).

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide (e.g., a fragment of human LILRB1 and/or LILRB2). The preparation can further include an adjuvant, such as Freund's complete or incomplete adjuvant, or a similar immunostimulatory agent.

Polyclonal antibodies can be prepared as described above by immunizing a suitable subject with LILRB1 and/or LILRB2 polypeptides, or antigenic peptides thereof (e.g., part of LILRB1 and/or LILRB2) as immunogens. The antibody titer in the immunized subject can be monitored over time by standard techniques, such as with an enzyme-linked immunosorbent assay (ELISA) using the immobilized LILRB1 and/or LILRB2 polypeptides or peptides. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target proteins, e.g., LILRB1 and/or LILRB2. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs. A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al., Nature, 321:522-525 (1986); Riechmann et al., Nature, 332:323-327 (1988); Verhoeyen et al., Science, 239:1534-1536 (1988); each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

The choice of human VH and VL domains to be used in making the humanized antibodies is very important for reducing immunogenicity. According to the so-called “best-fit” method, the sequence of the V domain of a mouse antibody is screened against the entire library of known human-domain sequences. The human sequence which is closest to that of the mouse is then accepted as the human FR for the humanized antibody (Sims et al., J. Immunol., 151:2296 (1993); Chothia et al., J. Mol. Biol., 196:901 (1987)).

In some embodiments, yeast display is performed to achieve affinity maturation. Details can be found, e.g., in Boder, E. T., et al. “Yeast surface display for screening combinatorial polypeptide libraries.” Nature Biotechnology 15.6 (1997): 553-557; Feldhaus, M. J., et al. “Flow-cytometric isolation of human antibodies from a nonimmune Saccharomyces cerevisiae surface display library.” Nature Biotechnology 21.2 (2003): 163-170; and Chao, G., et al. “Isolating and engineering human antibodies using yeast surface display.” Nature Protocols 1.2 (2006): 755-768; each of which is incorporated herein by reference in its entirety.

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

In some embodiments, humanization of the anti-LILRB1/LILRB2 antibodies or antigen-binding fragments thereof described herein is achieved in silicon, e.g., using MOE computer software.

Ordinarily, amino acid sequence variants of the human, humanized, or chimeric anti-LILRB1/2 antibody will contain an amino acid sequence having at least 75%, 80%, 85%, 90%, 95%, 96%, 97%, 98%, or 99% percent identity with a sequence present in the light or heavy chain of the original antibody.

Identity or homology with respect to an original sequence is usually the percentage of amino acid residues present within the candidate sequence that are identical with a sequence present within the human, humanized, or chimeric anti-LILRB1/2 antibody or fragment, after aligning the sequences and introducing gaps, if necessary, to achieve the maximum percent sequence identity, and not considering any conservative substitutions as part of the sequence identity.

Additional modifications to the anti-LILRB1/2 antibodies or antigen-binding fragments can be made. For example, a cysteine residue(s) can be introduced into the Fc region, thereby allowing interchain disulfide bond formation in this region. The homodimeric antibody thus generated may have any increased half-life in vitro and/or in vivo. Homodimeric antibodies with increased half-life in vitro and/or in vivo can also be prepared using heterobifunctional cross-linkers as described, for example, in Wolff et al. (Cancer Res. 53:2560-2565, 1993). Alternatively, an antibody can be engineered which has dual Fc regions (see, for example, Stevenson et al., Anti-Cancer Drug Design 3:219-230, 1989).

In some embodiments, a covalent modification can be made to the anti-LILRB1/2 antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about ±3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH may be used. For reviews, see Ausubel et al. (1989) Current Protocols in Molecular Biology, John Wiley & Sons, New York, N.Y, and Grant et al., Methods Enzymol., 153: 516-544 (1997).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986).

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

### Methods of Treatment

The antibodies or antibody or antigen-binding fragments thereof of the present disclosure can be used for various therapeutic purposes. In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of an antibody or antigen-binding fragment thereof disclosed herein to a subject in need thereof (e.g., a subject having, or identified or diagnosed as having, a cancer), e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy. In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the subject has a solid tumor or hematological cancer. In some embodiments, the cancer is squamous cell carcinoma of the head and neck (SCCHN), renal cell carcinoma (RCC), triple-negative breast cancer (TNBC), or colorectal carcinoma.

In some embodiments, the cancer is glioma, thyroid cancer, lung cancer, colorectal cancer, head and neck cancer, stomach cancer, liver cancer, pancreatic cancer, renal cancer, urothelial cancer, prostate cancer, testis cancer, breast cancer, cervical cancer, endomentrial cancer, ovarian cancer, or melanoma.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the antibody, antigen binding fragment, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody or an antigen binding fragment is an amount sufficient to ameliorate, stop, stabilize, reverse, inhibit, slow and/or delay progression of a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody or antigen binding fragment may vary, depending on, inter alia, patient history as well as other factors such as the type (and/or dosage) of antibody used.

Effective amounts and schedules for administering the antibodies, antibody-encoding polynucleotides, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, and/or compositions disclosed herein, the route of administration, the particular type of antibodies, antibody-encoding polynucleotides, antigen binding fragments, and/or compositions disclosed herein used and other drugs being administered to the mammal. Guidance in selecting appropriate doses for antibody or antigen binding fragment can be found in the literature on therapeutic uses of antibodies and antigen binding fragments, e.g., Handbook of Monoclonal Antibodies, Ferrone et al., eds., Noges Publications, Park Ridge, N.J., 1985, ch. 22 and pp. 303-357; Smith et al., Antibodies in Human Diagnosis and Therapy, Haber et al., eds., Raven Press, New York, 1977, pp. 365-389.

A typical daily dosage of an effective amount of an antibody is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, antigen-binding fragment thereof, or pharmaceutical composition (e.g., any of the antibodies, antigen-binding fragments, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody or antigen-binding fragment and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of antibodies or antigen-binding antibody fragments (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-PD1 antibody, an anti-PD-L1 antibody, an anti-LAG-3 antibody, an anti-TIGIT antibody, an anti-BTLA antibody, an anti-CTLA-4 antibody, or an anti-GITR antibody.

### Pharmaceutical Compositions and Routes of Administration

Also provided herein are pharmaceutical compositions that contain at least one (e.g., one, two, three, or four) of the antibodies or antigen-binding fragments described herein. Two or more (e.g., two, three, or four) of any of the antibodies or antigen-binding fragments described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antibodies or antigen-binding fragments described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies or antigen-binding fragments thereof (e.g., any of the antibodies or antibody fragments described herein) will be an amount that treats the disease in a subject (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 1 μg/kg to about 50 μg/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof for various uses as described herein.

### Examples

The invention is further described in the following examples, which do not limit the scope of the invention described in the claims.

### Example 1. Generation of Anti-LILRB1/2 Antibodies

A panel of antibodies that selectively bind human LILRB1 (RB1) and LILRB2 (RB2) were generated in BALB/c mice using a combination of recombinant human LILRB1-D1/D2 murine IgG2a fusion protein (with amino acid sequence set forth in SEQ ID NO: 823) and human LILRB2-D1/D2 murine IgG2a fusion protein (with amino acid sequence set forth in SEQ ID NO: 824); or a combination of human LILRB1-ECD-HIS (SinoBiological, Cat #:16014-H02H) and LILRB2-ECD-HIS (SinoBiological, Cat #: 14132-H02H) recombinant proteins as immunogens. The immunogens were resuspended with RIBI adjuvant in phosphate-buffered saline (PBS), and then administered by intraperitoneal injection.

Mice which showed desired serum titers were sacrificed. Spleens and bone marrow were collected. Splenocytes or plasma B cells isolated using mouse CD138+ Plasma Cell Isolation Kit (Miltenyi Biotec Inc., Cat #: 130-092-530) were separately processed to isolate single cells. The mRNAs were isolated from single cells. Nucleic acid sequences encoding the heavy chain variable region (VH) and light chain variable region (VL) were amplified by RT-PCR. The amplified PCR products encoding LILRB1/2-specific single-chain variable fragment (scFV) were engineered into yeast libraries.

The obtained yeast libraries were subject to 5 rounds of selection using Miltenyi MidiMACS™ system with biotinylated immunogens and fluorescence-activated cell sorting (FACS). Specifically, the libraries were treated sequentially with biotinylated immunogens, florescent streptavidin or Fc fusion immunogens, and florescent secondary antibodies. After the final round of selection, the yeast cells were plated.

Single yeast colonies were picked and inoculated into a 96-well plate with yeast growth medium for scFv production. Supernatants of the yeast culture were harvested and screened for protein binding using ELISA and Carterra®. Flow cytometry was also used to measure the binding to RB1 and RB2 expressing cells. Nucleic acid sequences encoding the heavy chain variable region (VH) and light chain variable region (VL) of LILRB1/2-specific antibodies were amplified by RT-PCR. Nucleic acid sequences encoding the VH and VL of the hits were obtained by Sanger sequencing. Afterwards, the sequences encoding VH and VL were cloned into a vector expressing human IgG1 (with mouse variable regions and human constant regions) with LALA mutations. The produced antibodies were subject to further screening and characterization.

To humanize the hits identified above, Molecular Operating Environment (MOE) was used to construct humanization template. Libraries were constructed by mutating in CDRs through modeling for affinity optimization and remove hypothetical manufacturing liabilities.

The humanization template and library sequences were synthesized by GenScript Biotech. DNA fragments of the humanization template and library sequences were amplified and introduced into yeast. Yeast screenings were performed similarly as the hit selection process as described above, except that more stringent threshold was used to achieve higher binding affinities.

### Example 2. Anti-LILRB1/2 Antibodies Blocked LILRB1 and LILRB2 Binding to HLA-G

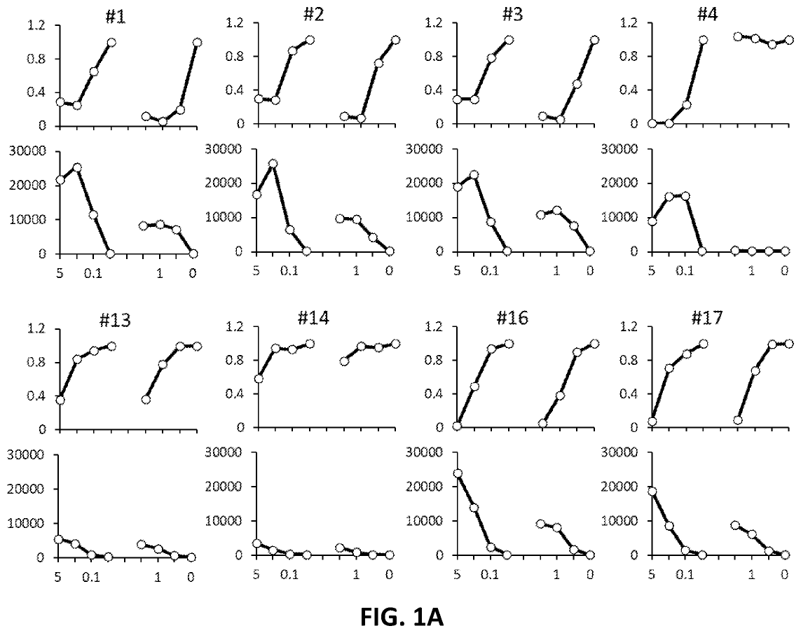

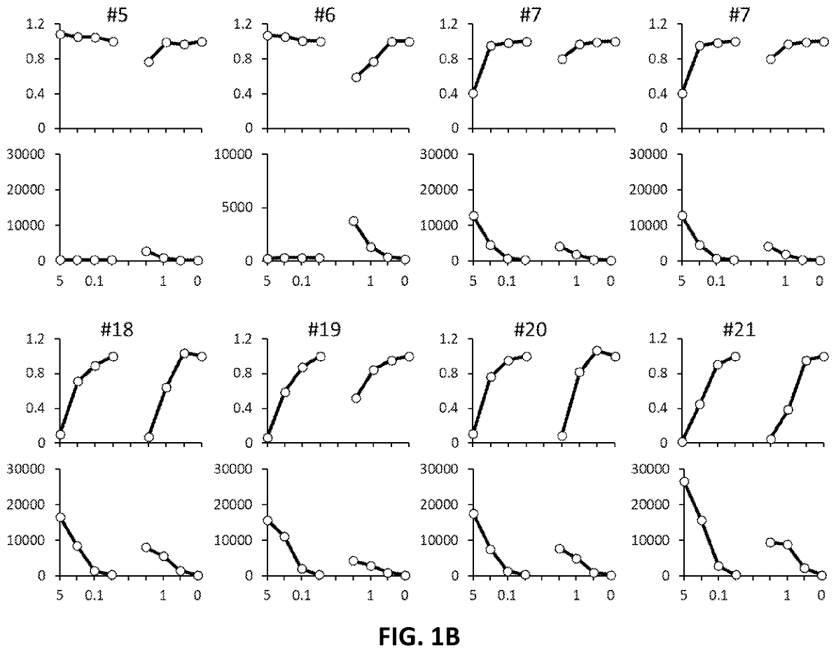

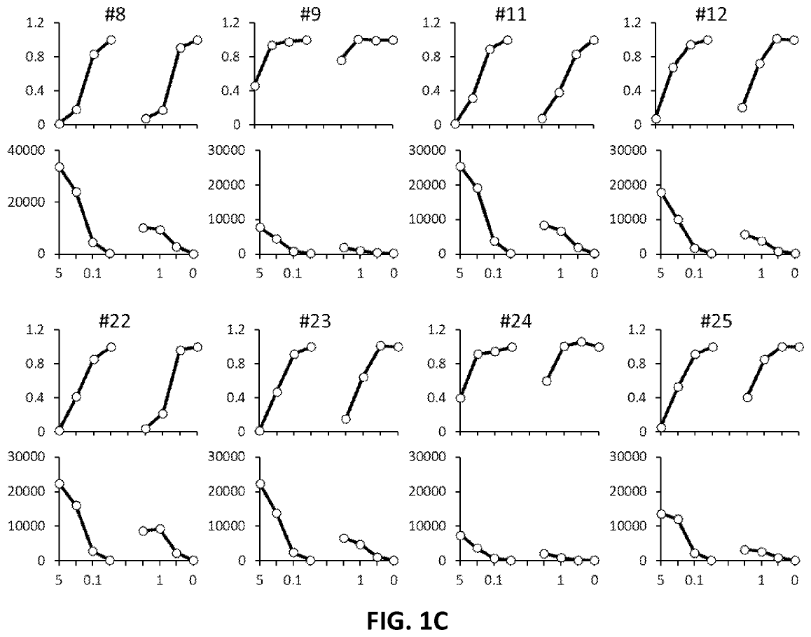

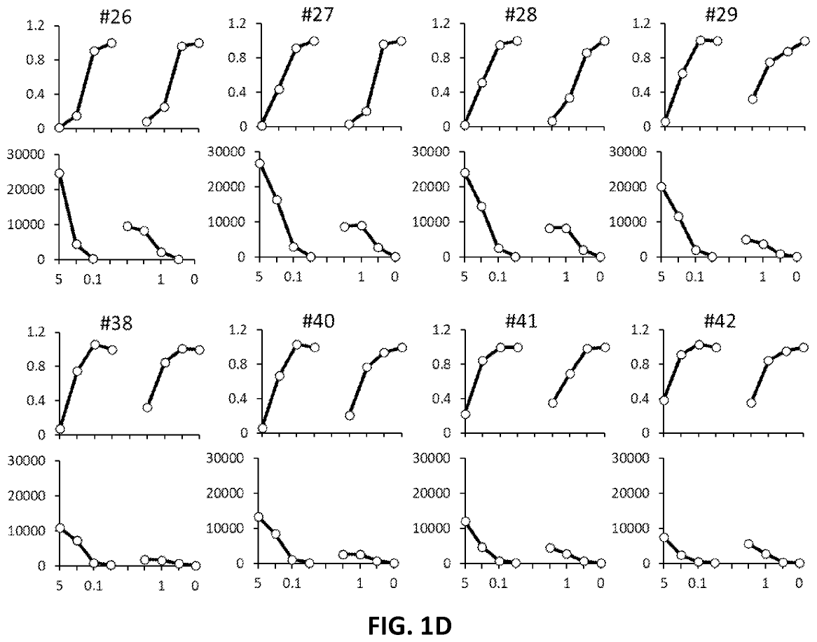

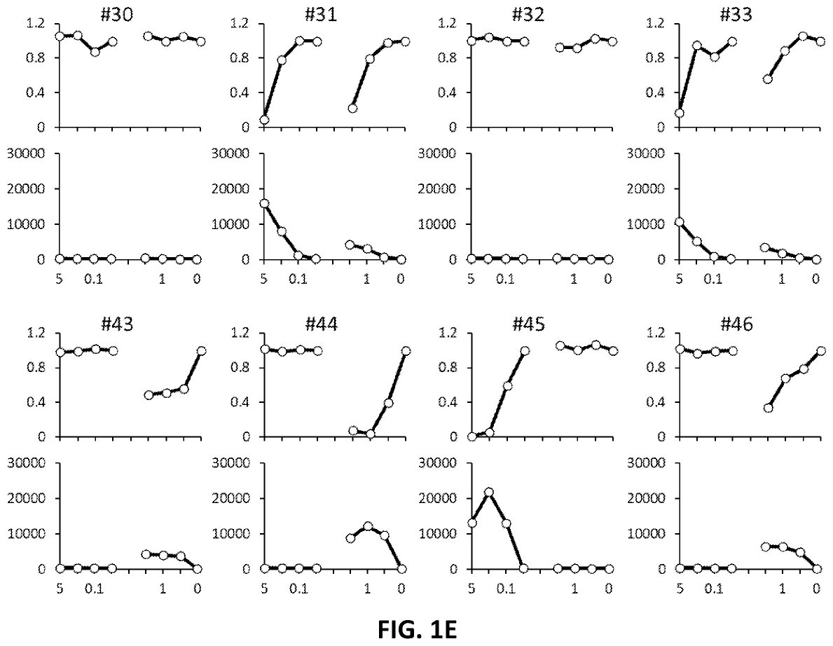

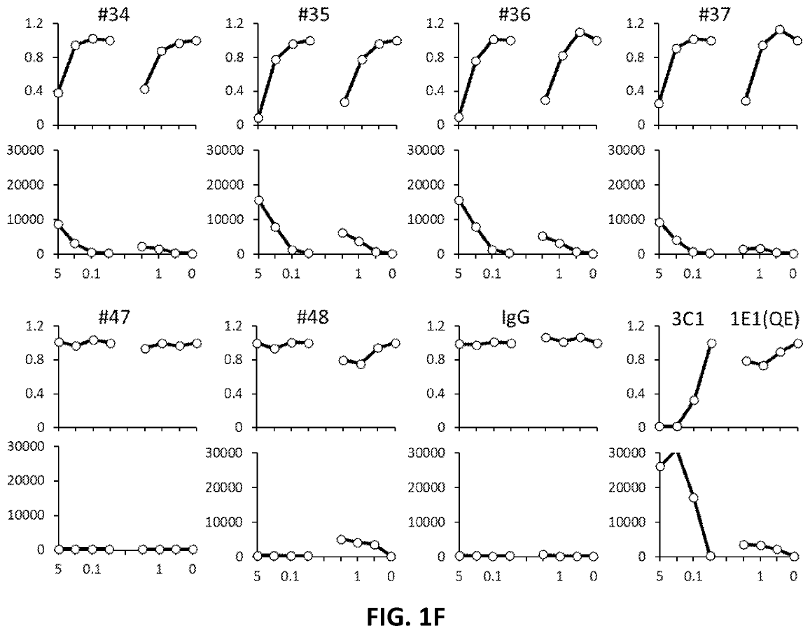

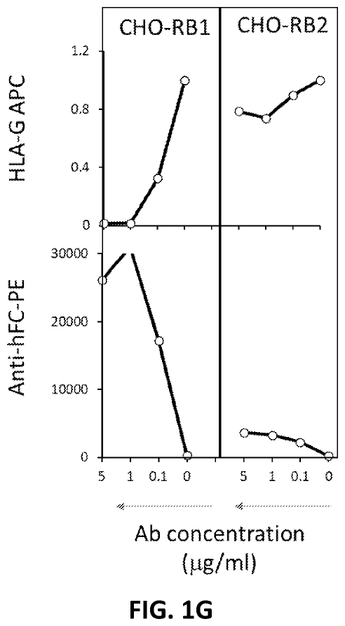

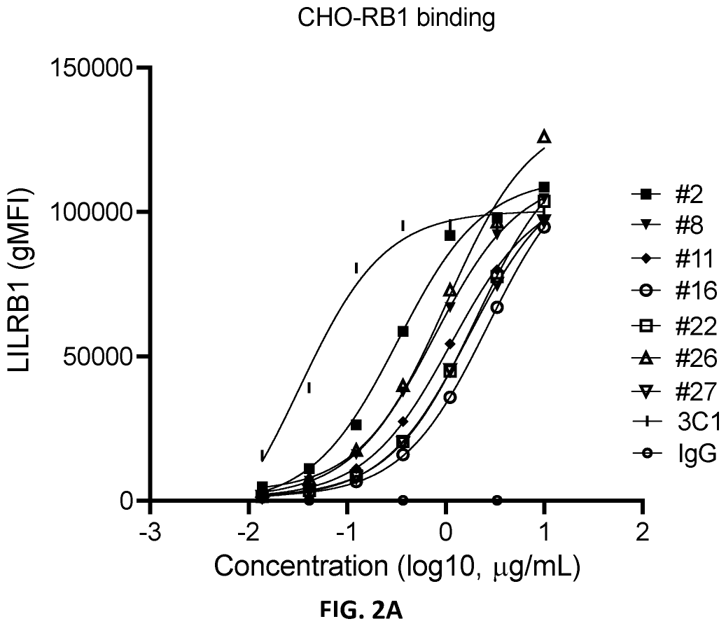

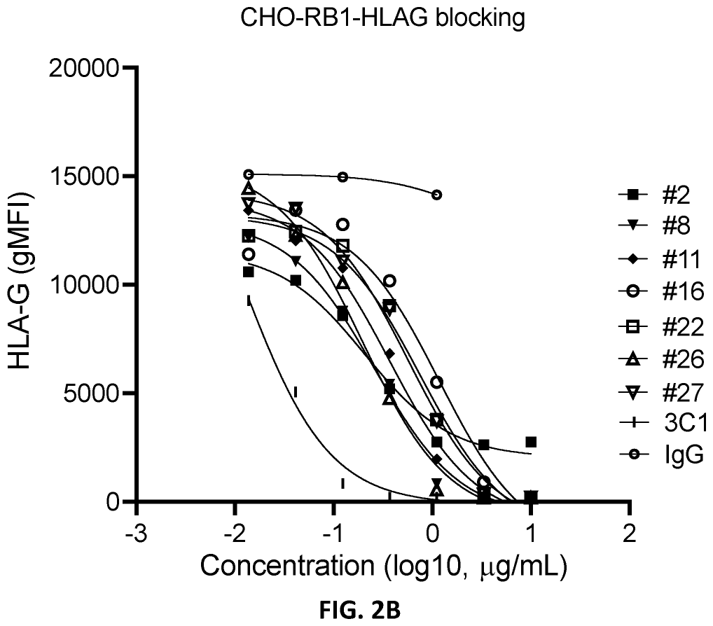

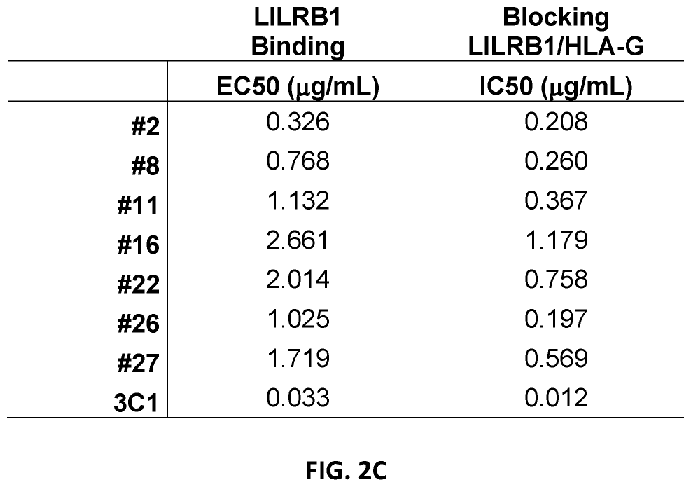

HLA-G/B2M/peptide trimer (Kactus Biosystems, Cat #: HLG-HM41C) and HLA-G monomer (Kactus Biosystems, Cat #: HLG-HE41F) were biotinylated by Enzymatic Protein Biotinylation kit (Sigma-Aldrich, Cat #: CS0008) and tetramerized with APC-conjugated Streptavidin (BioLegend, Cat #: 405243). The finally obtained tetrameric HLA-G complex was purified by Zeba™ Spin columns (Thermofisher, Cat #: 87768). Cell-based blocking assays were performed to determine whether the anti-LILRB1/2 antibodies can block the binding between LILRB1 over-expressed on CHO and tetramer of HLA-G/B2M/peptide trimer, and the binding between LILRB2 over-expressed on CHO and tetramer of HLA-G monomer (free-chain) using flow cytometry. 50 μL CHO-LILRB1 or CHO-LILRB2 cells (4×10⁴ cells) were added to each well in a 96-well U-bottom plate. Then, 50 μL titrated antibodies were added to final concentrations of 5 μg/mL, 1 μg/mL, 0.1 μg/mL and 0 μg/mL in FIGS. 1A-1F; or 10 μg/mL, 3.33 μg/mL, 1.11 μg/mL, 0.370 μg/mL, 0.123 μg/mL, 0.041 μg/mL, 0.0137 μg/mL, and 0 μg/mL in FIGS. 2A-2B. After an incubation at room temperature for 30 minutes, the plate was centrifuged, and cells were washed with PBS 3 times. Afterwards, 50 μL cocktail of Brilliant Violet™ 421-goat anti-hFc (200× final) and APC-tetramer of HAL-G trimer (2 μg/mL final) were added to each well. After mixing for 30 minutes at room temperature, cells were washed with PBS twice. Signals of Violet™ 421 (antibody binding) and APC (HLA-G binding) were determined by flow cytometry within the population of live cells (viability dye PI negative).

Alternatively, 50 μL Jurkat-LILRB1 or Jurkat-LILRB2 cells (5×10⁴ cells) were added to each well in a 96-well U-bottom plate. Then, 50 μL titrated antibodies were added to final concentrations of 30 μg/mL, 7.5 μg/mL, 1.875 μg/mL, 0.469 μg/mL, 0.117 μg/mL, 0.0293 μg/mL, 0.0073 μg/mL, 0.0018 μg/mL, and 0 μg/mL at room temperature for 30 minutes, the plate was centrifuged, and cells were washed with PBS 3 times. Afterwards, 50 μL cocktail of Brilliant Violet™ 421-goat anti-hFc (200× final) and APC-tetramer of HAL-G trimer (2 μg/mL final) were added to each well. After mixing for 30 minutes at room temperature, cells were washed with PBS twice. Signals of Violet™ 421 (antibody binding) and APC (HLA-G binding) were determined by flow cytometry within the population of live cells (viability dye PI negative).

As shown in FIGS. 1A-1F, when the concentration of the anti-LILRB1/2 antibodies or 3C1 increased, their binding LILRB1 (as shown by geometric MFI, or gMFI) increased while the signal of HLA-G decreased; when the concentration of anti-LILRB1/2 antibodies or 1E1(QE) increased, their binding LILRB2 increased while the signal of HLA-G decreased. The isotype control IgG1 bind to neither LILRB1 or LILRB2. The result led to the discovery of the anti-LILRB1/2 antibodies which bind to both LILRB1 or LILRB2 and block the interaction of HLA-G with LILRB1 or LILRB2.

As shown in FIGS. 2A-2B, when the concentration of the anti-LILRB1/2 antibodies or 3C1 increased, their binding LILRB1 (FIG. 2A) increased while the signal of HLA-G decreased (FIG. 2B). The isotype control IgG1 did not bind to LILRB1. As shown in FIG. 2C, the blocking efficiencies were calculated based on the signal of HLA-G normalized to the HLA-G signal when the antibody concentration was at 0 μg/mL. In addition, the binding EC50 and blocking IC50 concentrations were calculated based on the non-linear fitting curves generated by GraphPad Prism software.

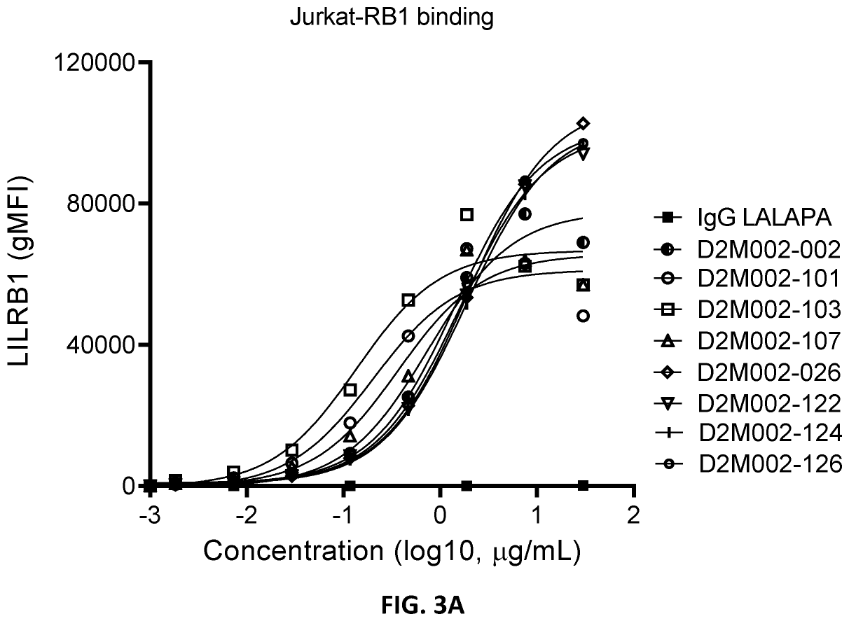

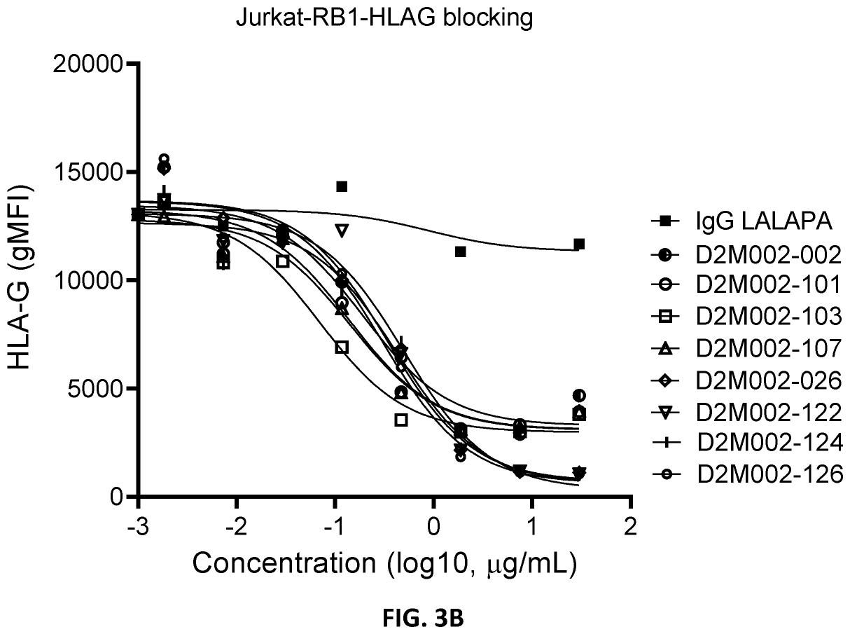

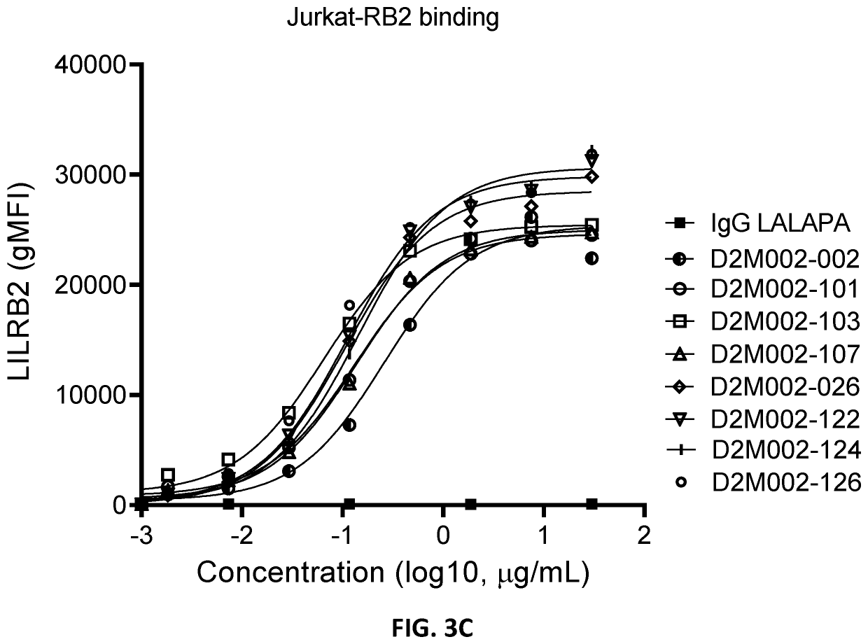

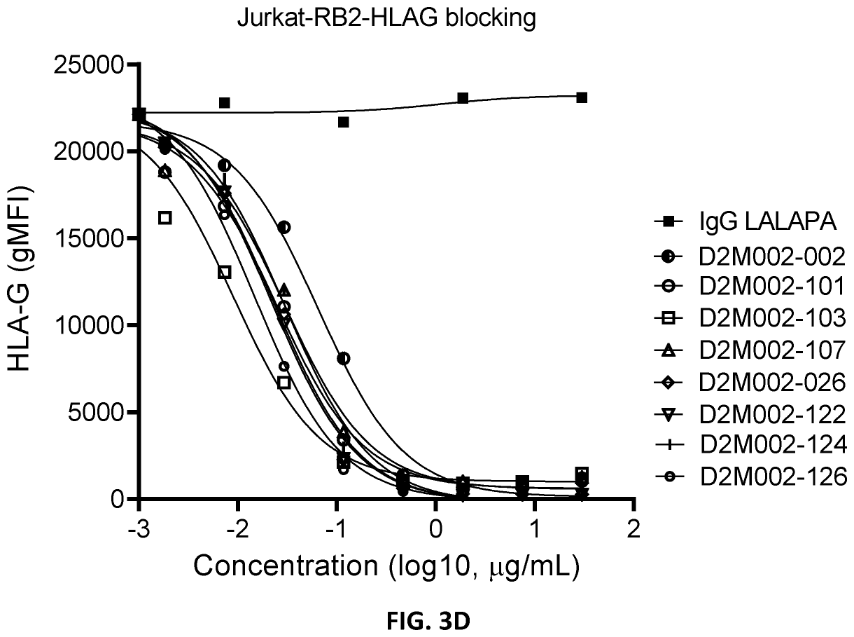

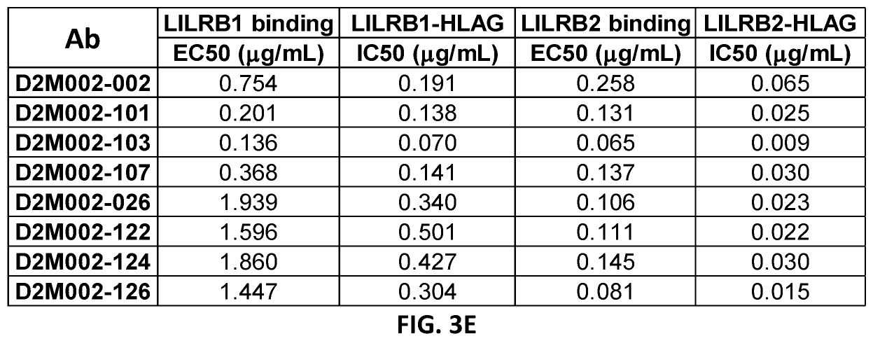

Humanized and optimized anti-LILRB1/2 antibodies also efficiently bound to LILRB1/2 and blocked the binding of HLA-G to LILRB1/2. As shown in FIGS. 3A-3B, when the concentration of the anti-LILRB1/2 antibodies increased, their binding LILRB1 (FIG. 3A) increased while the signal of HLA-G decreased (FIG. 3B). The isotype control IgG-LALA-PA did not bind to LILRB1. As shown in FIGS. 3C-3D, when the concentration of the anti-LILRB1/2 antibodies increased, their binding LILRB2 (FIG. 3C) increased while the signal of HLA-G decreased (FIG. 3D). The isotype control IgG-LALA-PA did not bind to LILRB2. The blocking efficiencies were calculated based on the signal of HLA-G normalized to the HLA-G signal when the antibody concentration was at 0 μg/mL. As shown in FIG. 3E, the binding EC50 and blocking IC50 concentrations were calculated based on the non-linear fitting curves generated by GraphPad Prism software.

The cell binding affinities and blocking efficiencies of humanized and optimized hits have been improved, represented by D2M002-103 from D2M002-002 lineage and D2M002-126 from D2M002-026 lineage.

### Example 3. Characterization of Anti-LILRB1/2 Antibodies Binding to LILRB1 and LILRB2

The binding kinetics of the antibodies were measured using Carterra® SPR imaging system (Carterra USA). Specifically, the anti-LILRB1/2 antibodies were captured by anti-human Fc (SouthernBiotech, Cat #: 2047-01) immobilized on a HC200M-Polycarboxylate chip (Carterra Bio, Cat #: HC200M) or a HC30M-Polycarboxylate chip (Carterra Bio, Cat #: HC30M) via amine-based coupling. Then, the corresponding monomeric antigen LILRB1-ECD-HIS (SinoBiological Cat #: 16014-H02H) or LILRB2-ECD-HIS (SinoBiological Cat #: 14132-H02H) were injected for 2-3 minutes, at 25° C. in running buffer. The dissociation was monitored for 5 or 10 minutes. Chip surface was regenerated between binding cycles with 10 mM Glycine HCl, pH 1.7. Binding kinetics was analyzed using software supplied by the manufacturer.

The binding kinetics of the antibodies were also measured using Gator Prime BLI (Biolayer Interferometry) system (Gator Bio, USA). Specifically, the anti-LILRB1/2 antibodies were loaded with HFC (Anti-hIgG Fc, Gator Bio, Cat #: 160003). Then, probes were incubated with corresponding monomeric antigens for 3-4 minutes, at 25° C. in K buffer (Gator Bio, Cat #: 120011). The dissociation was monitored for 5 or 10 minutes. The probes were regenerated between binding cycles with Regeneration Buffer (Gator Bio, Cat #: 120012). Binding kinetics was analyzed using software supplied by the manufacturer.

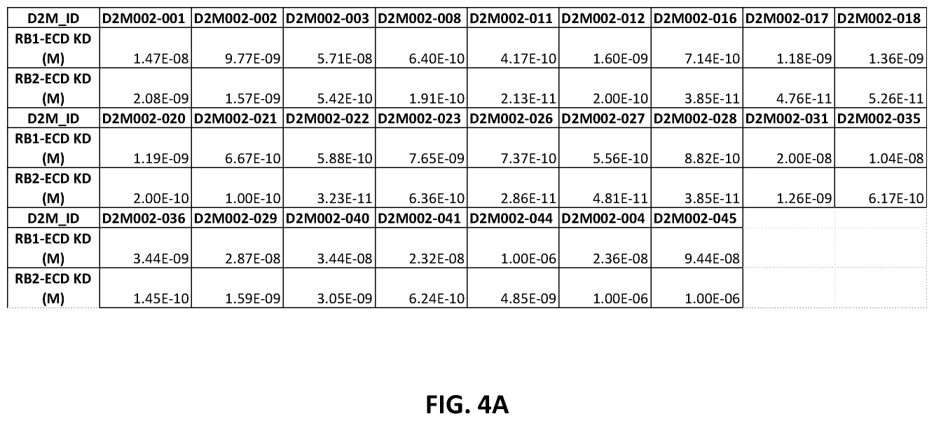

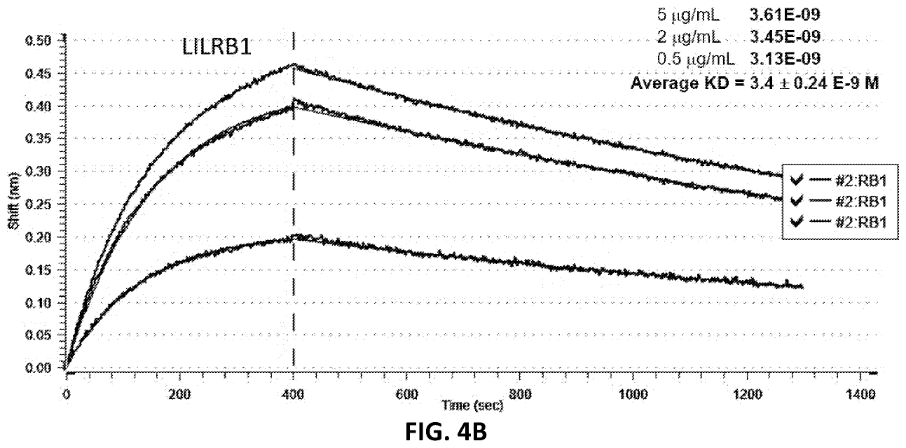

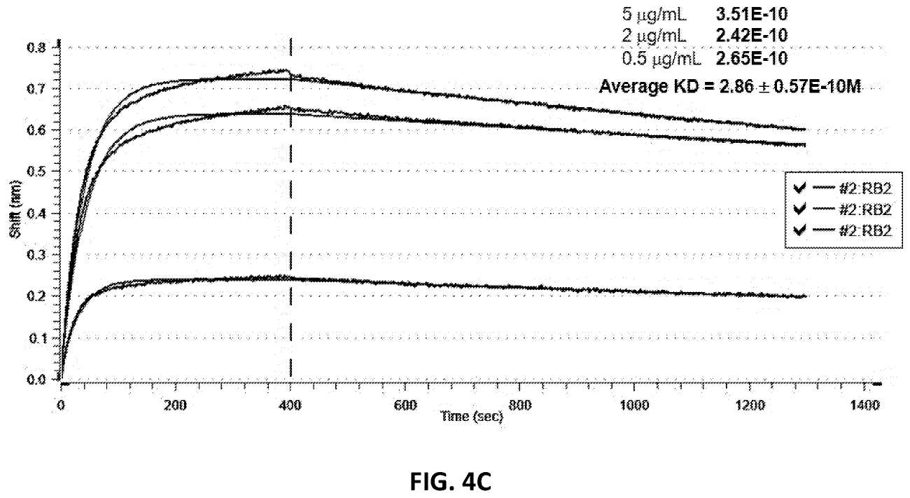

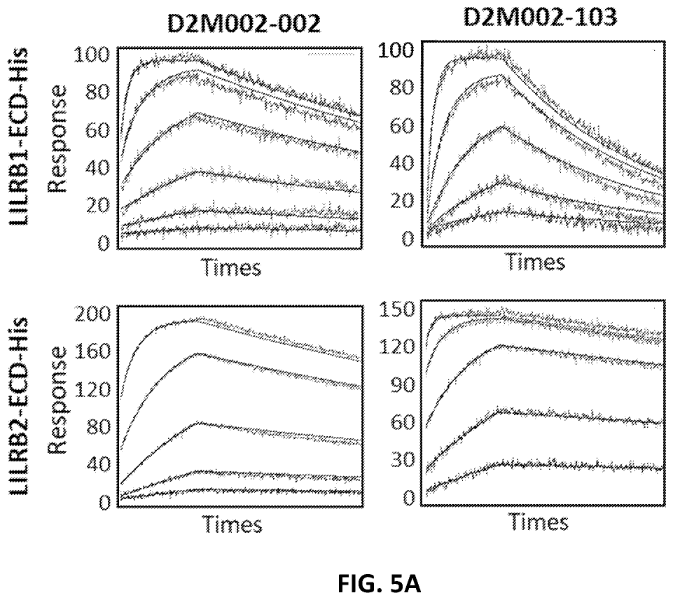

Binding affinities measured by the Carterra® system are shown in FIG. 4A. As shown in FIGS. 4B-4C, when the Gator system was used, D2M002-002 bound to LILRB1-ECD-HIS (SinoBiological Cat #: 16014-H02H) with an affinity of KD=3.4±0.24 nM, and bound to LILRB2-ECD-HIS (SinoBiological Cat #: 14132-H02H) with an affinity of KD=0.286±0.057 nM.

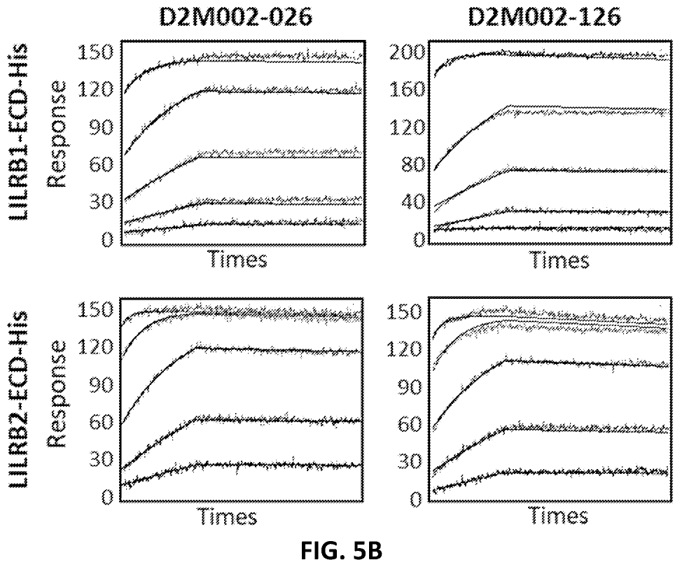

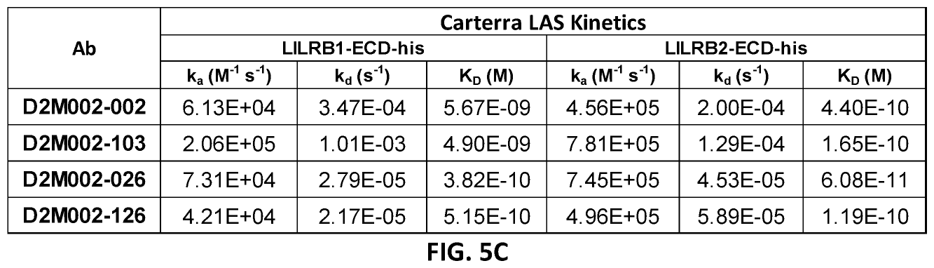

Humanized and optimized variants also showed improved binding affinity to both LILRB1 and LILRB2. As shown in FIGS. 5A-5B, the kinetic plots and fits of D2M002-103 and its parental D2M002-002, D2M002-126 and its parental D2M002-026, exhibited strong binding to LILRB1 and LILRB2. Binding affinities measured by the Carterra® system are shown in FIG. 5C. D2M002-126 exhibited sub nM affinities to both LILRB1 and LILRB2.

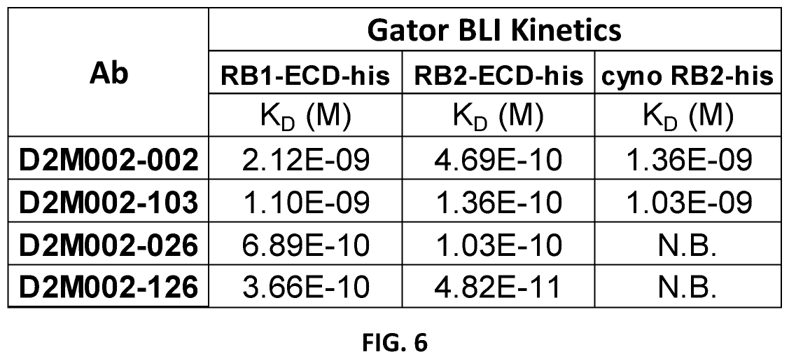

As shown in FIG. 6, D2M002 lineage but not D2M002-026 lineage can bind to cynomolgus monkey putative LILRB2, indicating that the two lineages have very distinct epitopes.

Cell-based antibody binding affinities were measured by incubating titrated anti-LILRB1/2 antibodies, a reference antibody, or an isotype control antibody (IgG1) with engineered CHO cells overexpressing human LILRB1 or LILRB2 (CHO-LILRB1 or CHO-LILRB2, respectively). Specifically, 50 μL CHO-LILRB1 or CHO-LILRB2 cells (4×10⁴ cells) were added to each well of a 96-well U-bottom plate. Then, 50 μL titrated anti-LILRB1/2 antibodies were added at final concentrations of 30 μg/mL, 10 μg/mL, 3.33 μg/mL, 1.11 μg/mL, 0.370 μg/mL, 0.123 μg/mL, 0.041 μg/mL, 0.0137 μg/mL, 0.0046 μg/mL, 0.0015 μg/mL and 0 μg/mL at room temperature. After an incubation for 30 minutes, the plate was centrifuged and cells were washed with PBS 3 times. Afterwards, 50 μL Brilliant Violet™ 421-goat anti-hFc (γ fragment specific, Jackson ImmunoResearch Laboratories Inc., Cat #:109-675-098, 200×) was added to each well. After mixing for 30 minutes at room temperature, cells were washed with PBS twice. Signals of Violet™ 421 were determined by flow cytometry (Attune™ CytPix™ ThermoFisher) within the population of live cells (viability dye PI negative).

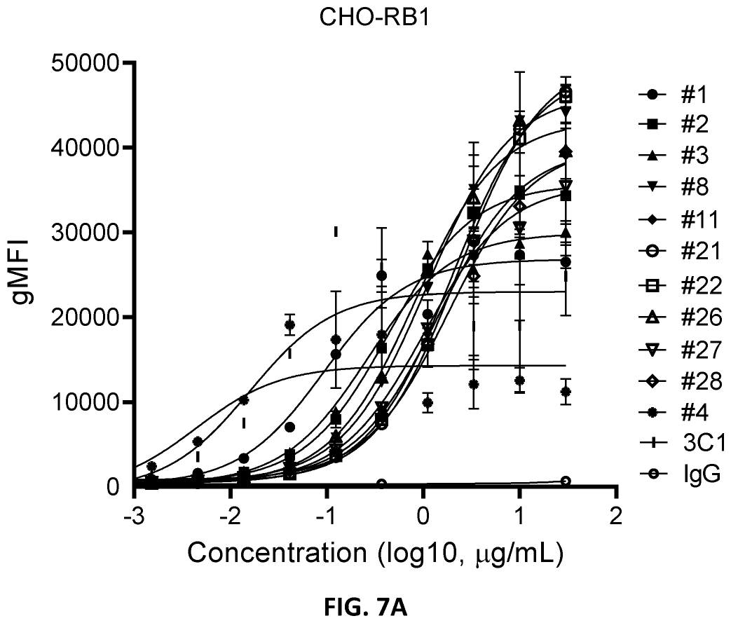

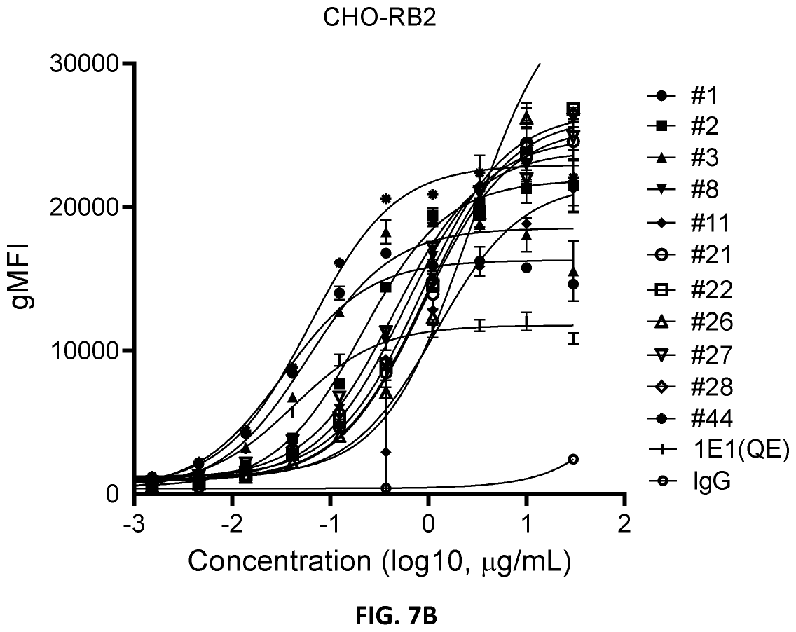

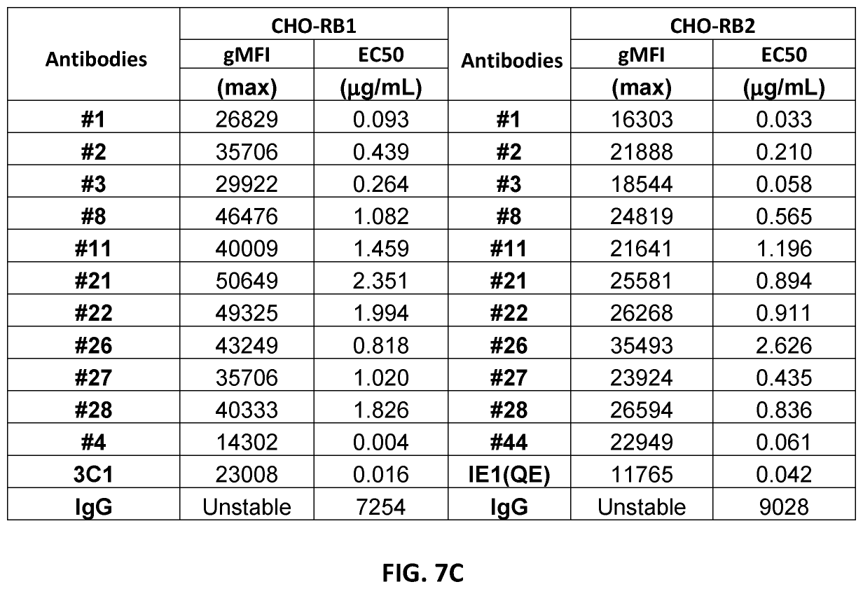

As shown in FIG. 7A, the tested anti-LILRB1/2 antibodies and the reference anti-LILRB1 antibody (3C1; heavy chain sequence: SEQ ID NO: 840; light chain sequence: SEQ ID NO: 839) exhibited strong binding abilities to LILRB1 expressed on CHO cells, whereas the isotype control antibody IgG1 did not. EC50 values (shown in the left panel of FIG. 7C) were calculated based on the non-linear fitting curves by GraphPad Prism software. As shown in FIG. 7B, the tested anti-LILRB1/2 antibodies and the reference anti-LILRB2 antibody 1E1(QE) (1E1; heavy chain sequence: SEQ ID NO: 838; light chain sequence: SEQ ID NO: 837) exhibited strong binding abilities to LILRB2 expressed on CHO cells, whereas isotype control antibody IgG1 did not. EC50 values (shown in right panel of FIG. 7C) were calculated based on the non-linear fitting curves by GraphPad Prism software.

### Example 4. Anti-LILRB1/2 Antibodies Released the Suppression of Anti-CD3 Activation in Jurkat Cells by Tumor Cells Expressing HLA-G

HLA-G expressed by tumor cells can suppress the activation of T cells through binding to LILRB1 or LILRB2. Cell-based functional assays were performed to determine whether anti-LILRB1/2 antibodies can block the suppressive signaling in Jurkat cells mediated by HLA-G on C1R tumor cells.

pLVX-NFAT-Luciferase-SV40 PolyA-PKG-Neomycin vector was designed to express Luciferase driven by a mini promoter incorporating three NFAT binding elements using pLVX-IRES-Puro vector (Takara Bio). pLVX-EF1a-OKTsFvB7.1-IRES-Puro was designed to express a membrane form of anti-CD3 scFv (OKT clone scFv, linker and membrane and cytosol fragment of gene B7.1/CD80). Lenti-X 293T were transfected with plasmids by Lenti-X Packaging Single Shots (Takara Bio) to generate fresh lentivirus. Jurkat-NFAT reporter cells were generated by transducing Jurkat cells with fresh lentivirus of pLVX-NFAT-Luciferase and selected with neomycin for one week. Single clones were isolated and identified by serial dilution culture. Jurkat-NFAT cells were transduced with fresh lentivirus of pLVX-EF1a-LILRB1-Puromycin or pLVX-EF1a-LILRB2-Puromycin and selected by puromycin for one week. Single clones were isolated and identified by serial dilution culture. Jurkat-NFAT-LILRB1 (Jurkat-RB1) or Jurkat-NFAT-LILRB2 (Jurkat-RB2) were cultured and maintained in complete medium with puromycin and neomycin. C1R-B7 lymphoma cells were transfected with pCMV3-HLA-G (Sino Biological) and selected with hygromycin. Single clones were isolated and identified by serial dilution culture. C1R or C1R-HLAG cells were transduced with fresh lentivirus of pLVX-EF1a-OKTsFvB7.1-IRES-Puro, named C1R-OKT3 or C1R-OKT3-HLAG cells.

To text fixed amount of hits, 50 μL Jurkat-RB1 or Jurkat-RB2 cells (5×10⁴ cells) were added to each well in a 96-well U-bottom plate. 25 μL antibodies (20 μg/mL) were added to a final concentration of 5 μg/mL and incubated for 30 minutes at room temperature. 25 μL C1R-OKT3-HLAG cells (1×10⁴ cells) were added to each well. After mixing, cells were co-cultured overnight for about 16 hours. Coculture of C1R-OKT3 with Jurkat-RB1 or Jurkat-RB2 were setup as controls without the HLA-G inhibitory signal. 100 μL luciferin (final 150 μg/mL) were added to each well, mixed and moved to a 96-well white flat-bottom plate. The plate was incubated for 10 minutes at room temperature, and then placed in a multimode plate reader (Varioskan™ Lux, Thermo Scientific) to measure luciferase activity.

To test titrated humanized and optimized hits, 50 μL Jurkat-RB1 or Jurkat-RB2 cells (5×10⁴ cells) were added to each well in a 96-well U-bottom plate. 25 μL titrated antibodies (20 μg/mL) were added to a final concentration of 30 μg/mL, 7.5 μg/mL, 1.875 μg/mL, 0.469 μg/mL, 0.117 μg/mL, 0.0293 μg/mL, 0.0073 μg/mL, 0.0018 μg/mL, and 0 μg/mL at room temperature. 5 μg/mL and incubated for 30 minutes at room temperature. 25 μL C1R-OKT3-HLAG cells (1×10⁴ cells) were added to each well. After mixing, cells were co-cultured overnight for about 16 hours. 100 μL luciferin (final 150 μg/mL) were added to each well, mixed and moved to a 96-well white flat-bottom plate. The plate was incubated for 10 minutes at room temperature, and then placed in a multimode plate reader (Varioskan™ Lux, Thermo Scientific) to measure luciferase activity.

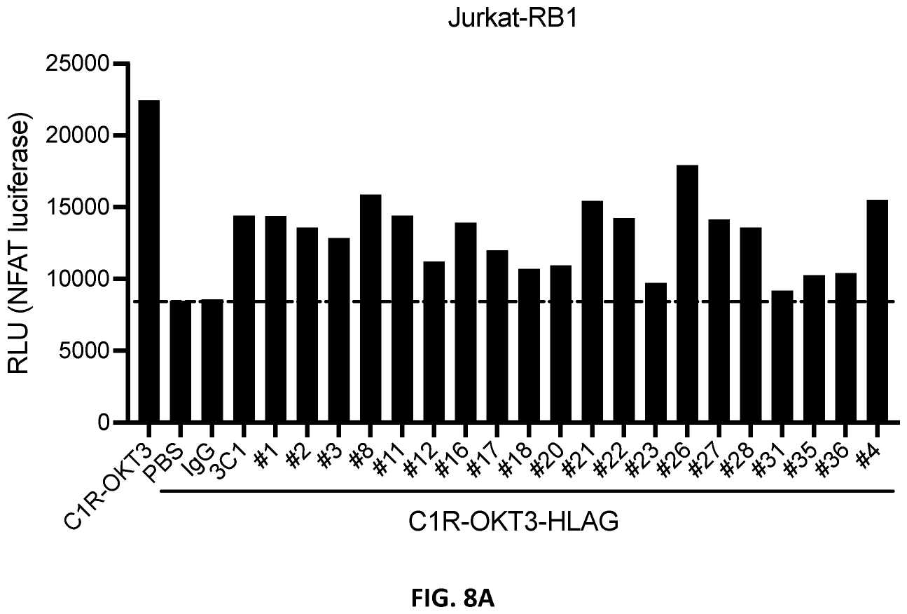

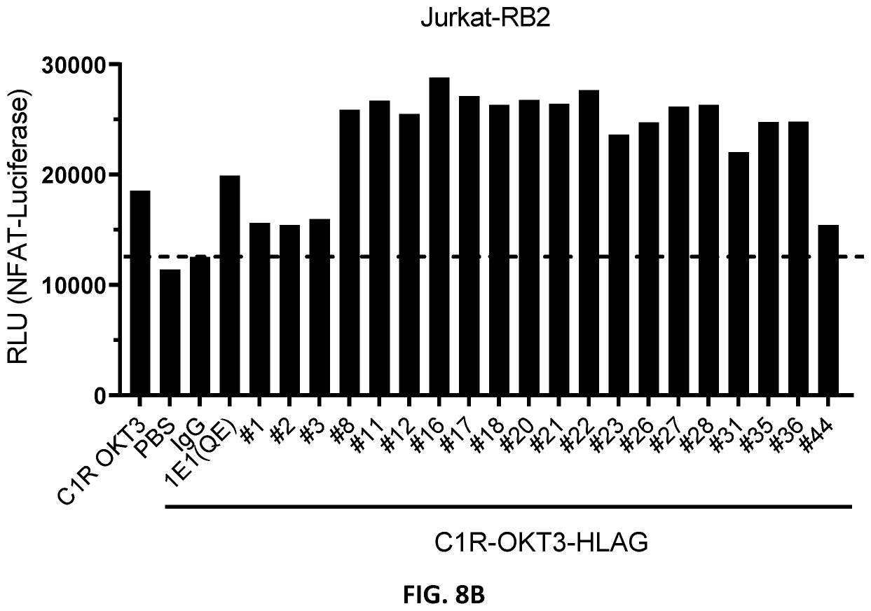

As shown in FIGS. 8A, luciferase signal of C1R-OKT3-HLAG co-cultured with Jurkat-RB1 decreased as compared to that of the co-culture of C1R-OKT3 and Jurkat-RB1, demonstrating HLA-G suppressed the activation of Jurkat-RB1 cells under anti-CD3 stimulation. The luciferase signal of C1R-OKT3-HLAG and Jurkat-RB1 increased when Jurkat-RB1 cells were treated with 5 μg/mL anti-LILRB1/2 antibodies or the reference antibody 3C1, as compared to the luciferase signal when Jurkat-RB1 cells were treated with the IgG1 isotype control or in control wells where no antibody was added.

As shown in FIGS. 8B, the luciferase signal of C1R-OKT3-HLAG co-cultured with Jurkat-RB2 decreased as compared to that of the co-culture of C1R-OKT3 and Jurkat-RB2, demonstrating HLA-G suppressed the activation of Jurkat-RB2 cells under anti-CD3 stimulation.

The luciferase signal of C1R-OKT3-HLAG and Jurkat-RB2 increased when Jurkat-RB2 cells were treated with 5 μg/mL anti-LILRB1/2 antibodies or the reference antibody 1E1(QE), as compared to the luciferase signal when Jurkat-RB2 cells were treated with the IgG1 isotype control or in control wells where no antibody was added.

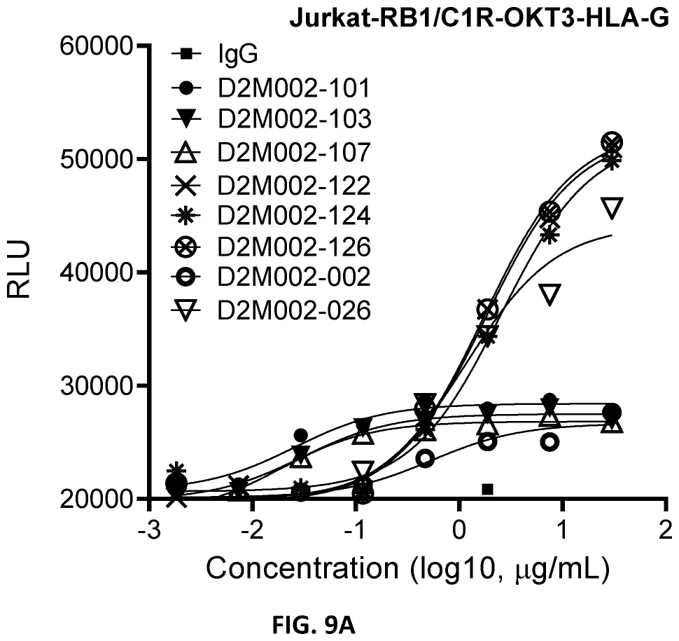

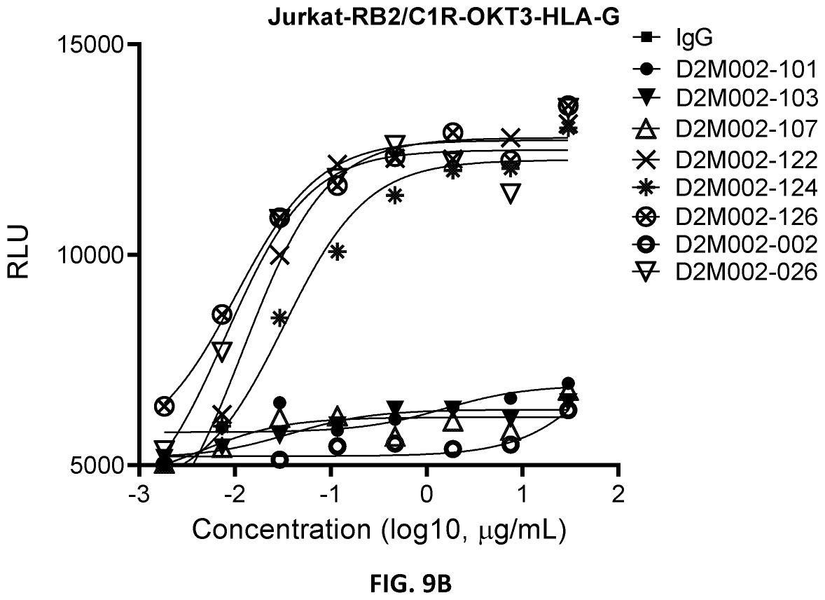

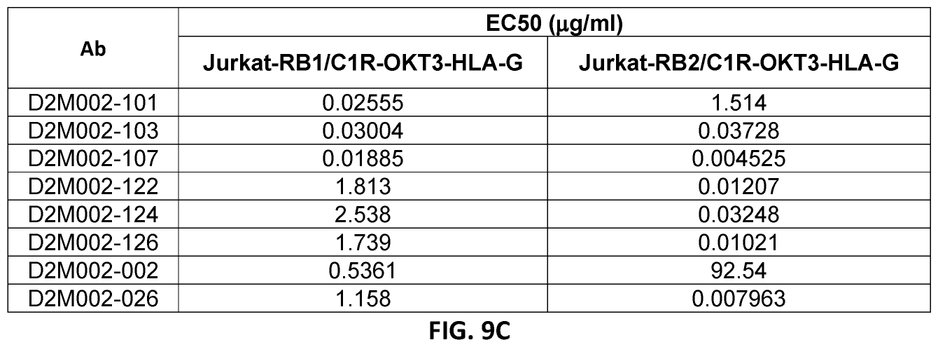

Humanized and optimized anti-LILRB1/2 antibodies also efficiently restored the T cell activation suppressed by HLA-G in Jurkat T expressing LILRB1 or LILRB2. As shown in FIG. 9A-9B, the luciferase signal of C1R-OKT3-HLAG co-cultured with Jurkat-RB1 or Jurkat-RB2 increased over anti-LILRB1/2 in a dose-dependent manner, demonstrating the anti-LILRB1/2 antibodies reversed the activation of Jurkat-RB1 or -RB2 cells suppressed by the HLA-G. The isotype control IgG1-LALA-PA did not change the luciferase signal in Jurkat-RB1 or -RB2 cells activated by C1R-OKT3-HLAG cells. EC50 values (shown in FIG. 9C) were calculated based on the non-linear fitting curves by GraphPad Prism software.

Although the humanized and optimized D2M002-002 variants have improved potency significantly over parental D2M002-002, the results indicate that D2M002-026 variants are more efficacious and potent than D2M002-002 variants in release HLA-G suppression in this assay, and D2M002-026 variants can lead to much higher extent of activation than D2M002-002 variants do.

### Example 5. Anti-LILRB1/2 Antibodies Promoted the Activation of Human PBMCs by Low LPS

Both LILRB1 and LILRB2 can be expressed in human monocytes. Human PBMC-based functional assays were performed to evaluate the effects of anti-LILRB1/2 antibodies on monocyte activation. Specifically, human PBMCs were purified from fresh blood of heathy donors (Research Blood Components, LLC) using Ficoll®-Pague (Cytiva, Cat #: 17544202) gradient centrifuge. 50 μL PBMCs (2×10⁵ cells) were added to each well in a 96-well U-bottom culture plate. 25 μL anti-LILRB1/2 antibodies, the reference antibodies 3C1 or 1E1(QE), and the IgG1 isotype control (20 μg/mL) were added. The plate was incubated at room temperature for 10 minutes. After mixing to a final concentration of 5 μg/mL, 25 μL LPS (E. coli 0111:B4, Sigma, Cat #: L2630-10MG, 200 μg/mL) were added to a final concentration of 50 ng/mL. The plate was incubated in culture hood for 24 hours.

To examine the effects of titrated antibodies, 50 μL PBMCs (2×10⁵ cells) were added to each well in a 96-well U-bottom culture plate. 25 μL titrated anti-LILRB1/2 antibody (D2M002-126) or a reference antibody (Hz73D1.v1; heavy chain sequence: SEQ ID NO: 1062, light chain sequence: SEQ ID NO: 1061), or an isotype control (IgG1-LALAPA) was added to a final concentration of 15 μg/mL, 5 μg/mL, 1.667 μg/mL, 0.556 μg/mL, 0.185 μg/mL, 0.0617 μg/mL, 0.0206 μg/mL, 0.0069 μg/mL, and 0 μg/mL. The plate was incubated at room temperature for 10 minutes. 25 μL LPS (E. coli 0111:B4, Sigma, Cat #: L2630-10MG, 200 μg/mL) were added to a final concentration of 50 ng/mL. The plate was incubated in culture hood for 24 hours. PBMC from three healthy donors were used and performed independently.

To assess the activation of human monocytes in PBMC culture, cytokine levels of TNFα and IL10 in the cell culture supernatants were quantified by ELISA kits of human TNFα and IL10 (BioLegend, Cat #: 430204 and #430604).

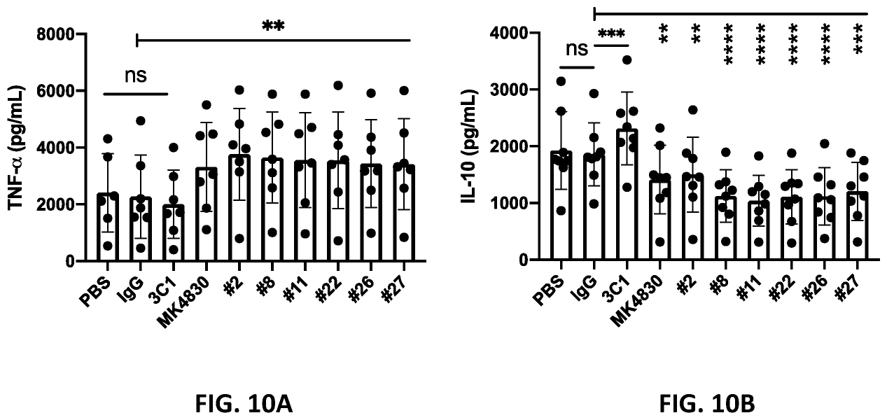

As shown in FIGS. 10A-10B, anti-LILRB1/2 antibodies and 1E1(QE) enhanced the production of TNFα (FIG. 10A) and inhibited the production of IL-10 (FIG. 10B) in PBMCs stimulated by low LPS stimulation across multiple donors, whereas 3C1 and the IgG1 isotype control did not promote TNFα production. 3C1 promoted the production of IL-10 comparing to IgG1 isotype.

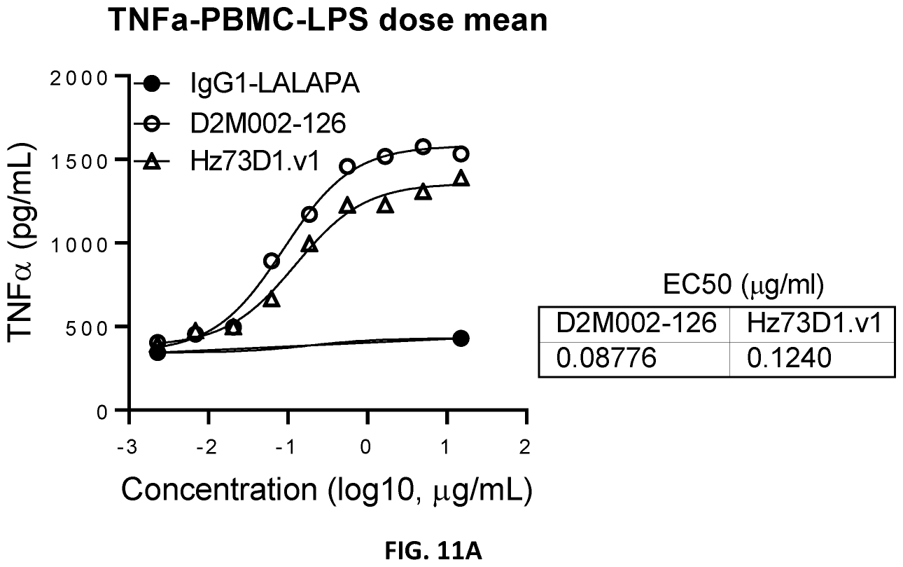

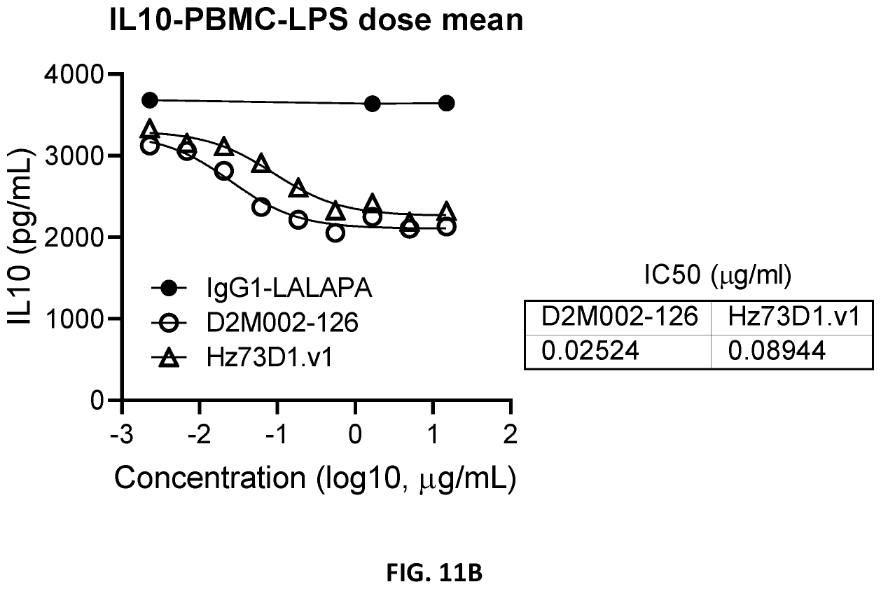

With respect to humanized and optimized anti-LILRB1/2 antibodies, D2M002-126 enhanced the PBMC activation by low LPS in a dose-dependent manner, more efficiently than the reference antibody, Hz73D1.v1. As shown in FIGS. 11A-11B, anti-LILRB1/2 antibody D2M002-126 and Hz73D1.v1 enhanced the production of TNFα (FIG. 11A) and inhibited the production of IL-10 (FIG. 11B) in PBMCs stimulated by low LPS, in a dose-dependent manner, whereas the IgG1 isotype control did not change the productions of TNFα and IL-10. D2M002-126 exhibits stronger effects to enhance the production of TNFα and inhibited the production of IL-10 across multiple donors compared to Hz73D1.v1.

### Example 6: Anti-LILRB1/2 Antibodies Promoted Inflammatory Macrophage Derived from Human Monocytes

As anti-LILRB1/2 antibody blockade amplified monocyte activation in response to LPS, the differentiation and function of human monocyte-derived macrophages (hMDMs) in the presence of anti-LILRB1/2 antibodies and M-CSF (macrophage colony-stimulating factor) were investigated. Specifically, human monocytes were isolated from PBMCs of heathy donors by Human CD14+ Monocytes Isolation Kit (MojoSort™ BioLegend, Cat #: 480038). 50 μL human monocytes (1×10⁵ cells) supplemented with 100 ng/mL M-CSF (BioLegend, Cat #: 574806) were added to a 96-well flat-bottom culture plate and then 50 μL antibodies (2 μg/mL) were added. hMDMs were generated by treating human monocytes with human M-CSF (hM-CSF) in the presence or absence of the anti-LILRB1/2 antibodies, the reference antibodies, or the IgG1 isotype control for 6 days. To assess the macrophage phenotype, hMDMs were detached by Accutase® (BioLegend, Cat #: 480038) and stained with APC anti-human CD14 (BioLegend, Cat #: 301808), BV605 anti-human CD11B (BioLegend, Cat #: 301332), FITC anti-human CD86 (BioLegend, Cat #: 374204), PerCP-Cy5.5 anti-human CD163 (BioLegend, Cat #: 326512), APC-Cy7 anti-human CD206 (BioLegend, Cat #: 321120), or BV785 anti-human PD-L1(BioLegend, Cat #: 124321). The expression profile of each phenotypic marker was quantified by flow cytometry (Attune™).

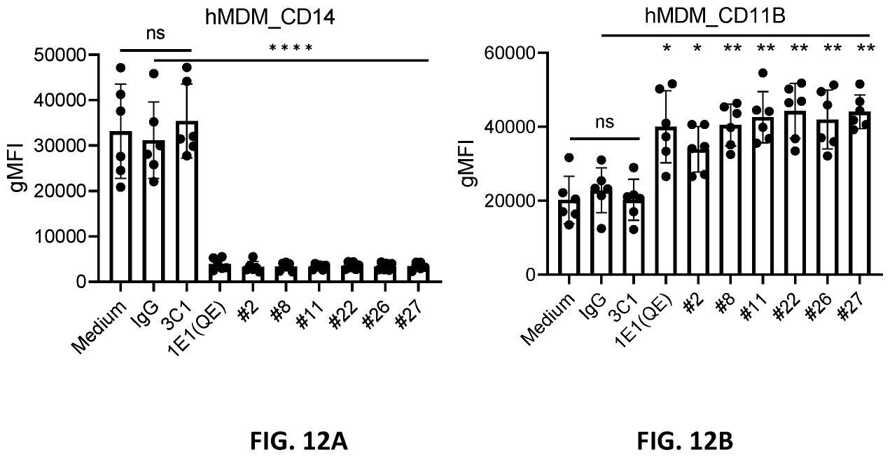

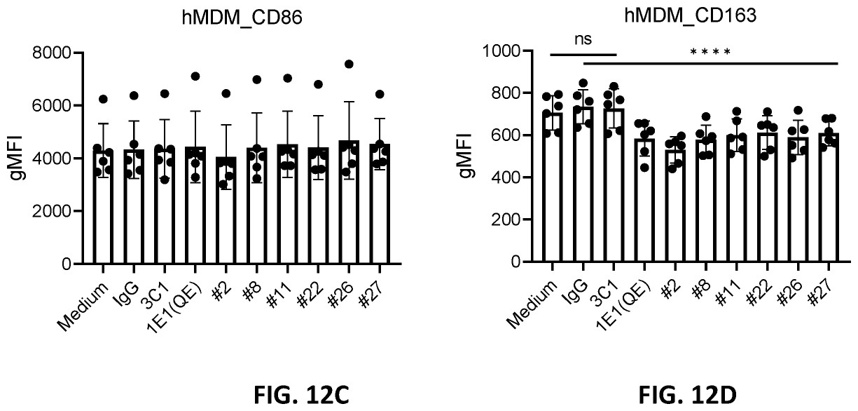

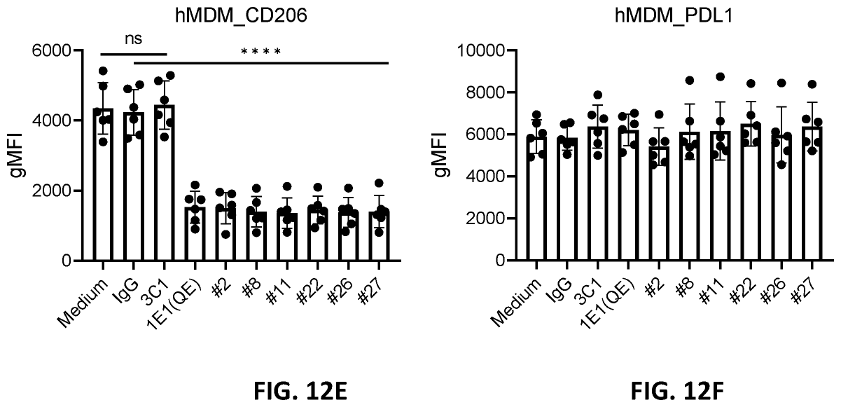

As shown in FIGS. 12A-12F, anti-LILRB1/2 antibodies and 1E1(QE) decreased the expression of CD14, anti-inflammatory makers CD163 and CD206; and increased the expression of CD11B across multiple donors. Similar to the IgG1 isotype control, 3C1 did not change the differentiation of hMDMs.

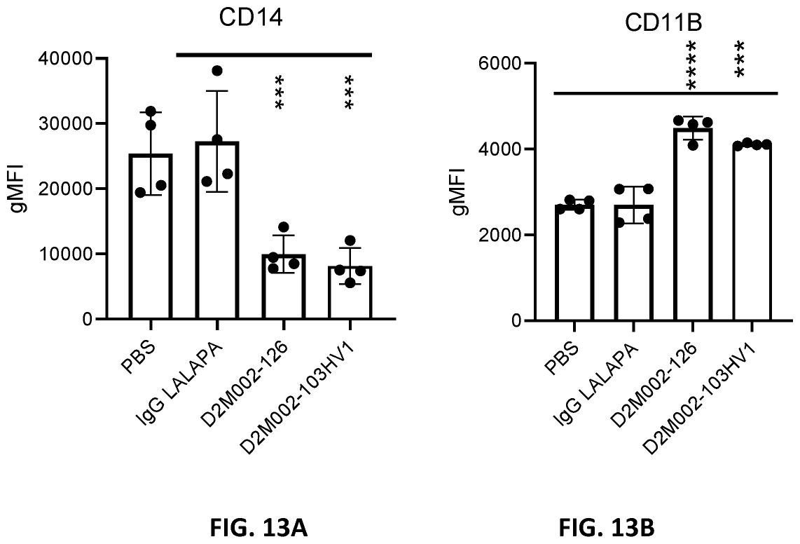

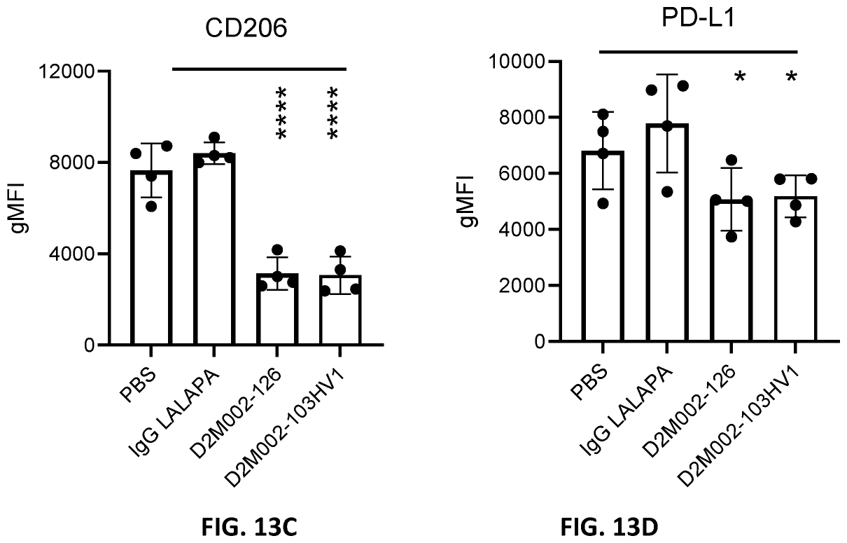

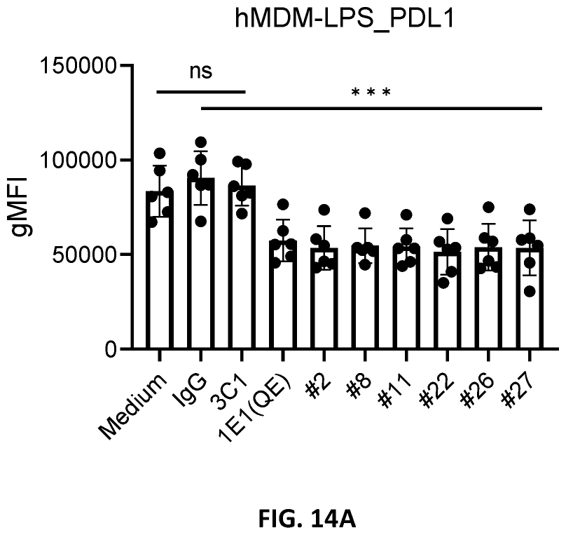

Humanized and optimized anti-LILRB1/2 also promoted inflammatory macrophage derived from human monocytes. As shown in FIGS. 13A-13D, D2M002-126 and D2M002-103HV1 decreased the expression of CD14, anti-inflammatory maker CD206 and immunosuppressive marker PD-L1 (an inhibitory immune checkpoint molecule); and increased the expression of CD11B across multiple donors compared to the isotype control (IgG1-LALAPA). The results indicate that the anti-LILRB1/2 antibodies polarized human monocytes to pro-inflammatory macrophages.

To determine whether hMDMs can display a differential response to LPS stimulation as was shown for monocytes, hMDMs in another plate with the same treatment were stimulated by replacing with fresh medium supplemented with 100 ng/mL LPS (E. coli 0111::B4, Sigma, #L2360) for 24 hours. Cytokines of TNFα and IL10 were quantified in the culture supernatants by ELISA kits against human TNFα and IL10 (BioLegend, Cat #: 430204 and #430604). Stimulated hMDMs were detached by Accutase and stained with APC anti-human CD14, BV605 anti-human CD11B, FITC anti-human CD86, PerCP-Cy5.5 anti-human CD163, APC-Cy7 anti-human CD206, or BV785 anti-human PD-L1. The expression profile of each phenotypic marker was quantified by flow cytometry (Attune™).

As shown in FIGS. 14A-14C, in the presence of the anti-LILRB1/2 antibodies and 1E1(QE), differentiated hMDMs consistently had a less expression level of immunosuppressive marker of PD-L1, increased TNFα and decreased IL10 secretion upon LPS stimulation across multiple donors. As shown in FIGS. 15A-15B, D2M002-126 and D2M002-103HV1 promoted differentiated hMDMs to produce more TNFα and less IL10 secretion upon LPS stimulation across multiple donors.

### Example 7. Anti-LILRB1/2 Antibodies Reprogramed Tumor-Associated Macrophages Induced by Tumor Cells and Promoted Anti-Tumor Immunity

Human monocytes were isolated from PBMCs of healthy voluntary donors by Human CD14+ Monocytes Isolation Kit. Tumor-associated macrophages (TAMs) were generated by co-culturing the human monocytes and tumor cells in the presence or absence of the anti-LILRB1/2 antibodies, the reference antibodies, or IgG1 isotype control for 6 days. 50 μL mixture of human monocytes (1×10⁵ cells) and human breast cancer cells MDA-MB-231 (1×10³ cells) were added to a 96-well flat-bottom culture plate and then 50 μL antibodies (2 μg/mL) were added to a final concentration of 1 μg/mL. To assess the macrophage phenotype, TAMs and tumor cells were detached by Accutase and stained with APC anti-humanCD14, BV605 anti-human CD11B, FITC anti-human CD86, PerCP-Cy5.5 anti-human CD163, APC-Cy7 anti-human CD206, or BV785 anti-human PD-L1. The expression profile of each phenotypic marker in TAMs and proportion of tumor cells were quantified by flow cytometry (Attune™).

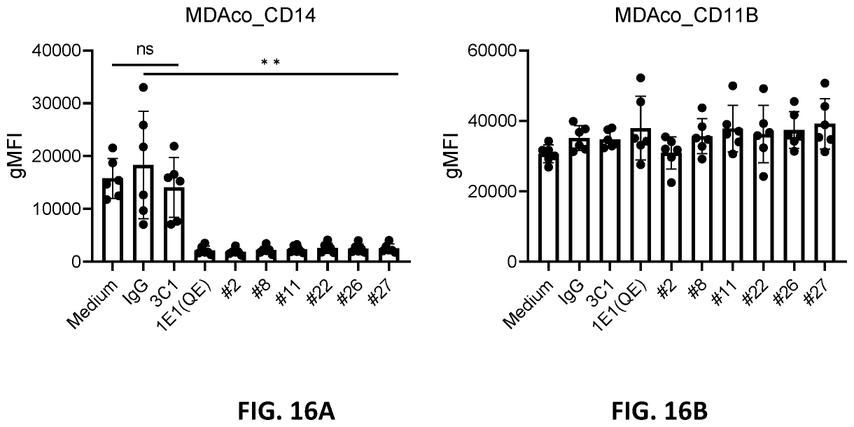

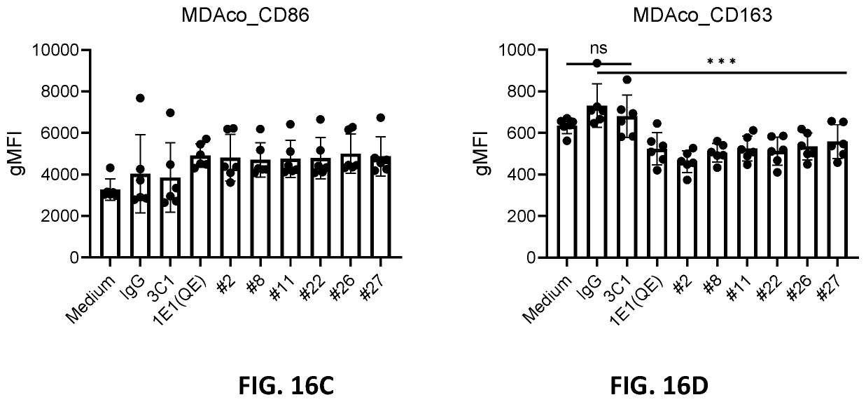

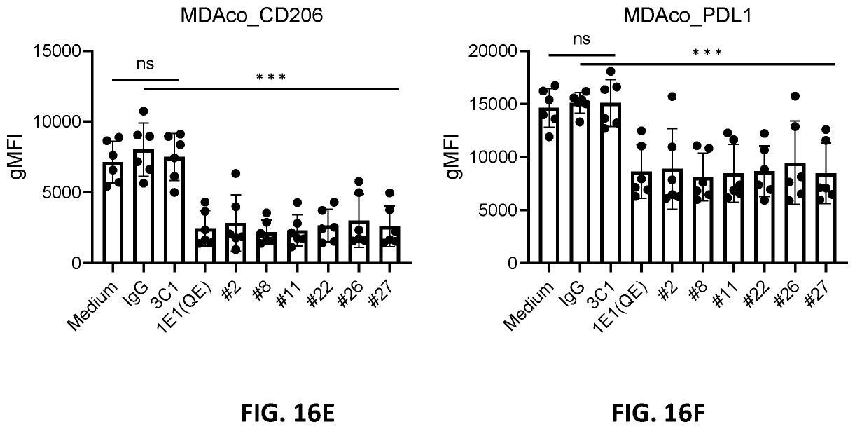

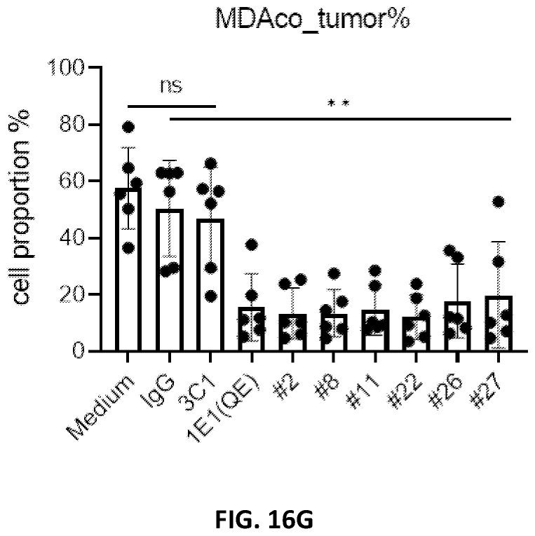

As shown in FIGS. 16A-16F, anti-LILRB1/2 antibodies and 1E1(QE) decreased the expression of CD14, anti-inflammatory makers CD163 and CD206, and immunosuppressive marker PD-L1 (an inhibitory immune checkpoint molecule) in TAMs across multiple donors. Similar to the IgG1 isotype control, 3C1 did not change the differentiation of TAMs. The results indicate that the anti-LILRB1/2 antibodies reprogramed TAMs. Meanwhile, there were significantly less tumor cells in the co-culture where the anti-LILRB1/2 antibodies and 1E1(QE) were present (FIG. 16G).

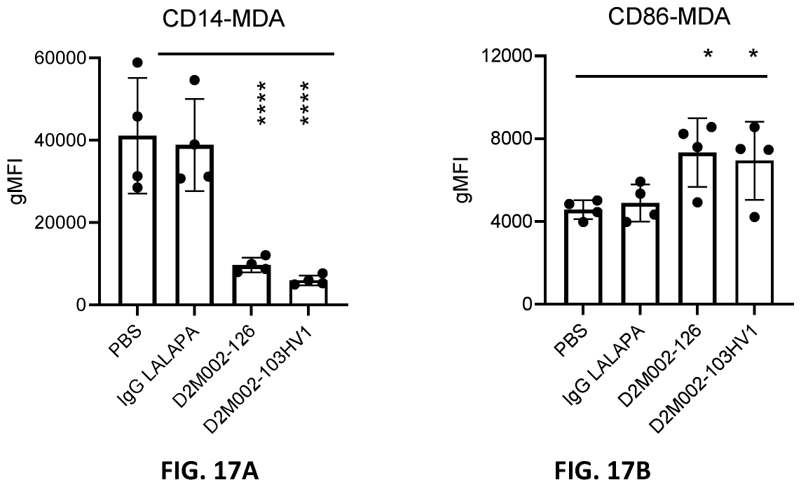

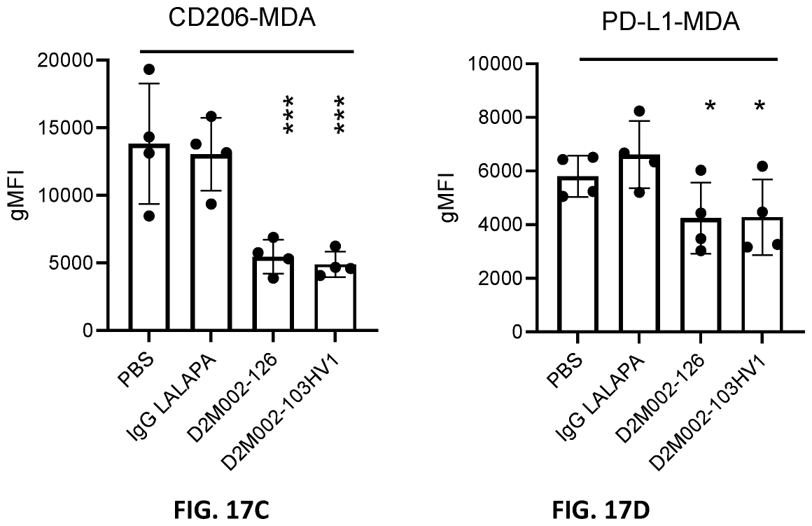

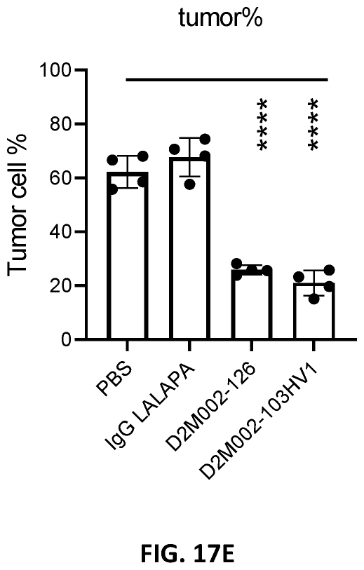

Humanized and optimized anti-LILRB1/2 antibodies can also reprogram tumor-associated macrophages induced by tumor cells, promote anti-tumor immunity, and inhibit tumor growth in vitro. As shown in FIGS. 17A-17D, D2M002-126 and D2M002-103HV1 decreased the expression of CD14, anti-inflammatory maker CD206, and immunosuppressive marker PD-L1 (an inhibitory immune checkpoint molecule) in TAMs across multiple donors as compared to the isotype control IgG1-LALA-PA. As shown in FIGS. 17E, the anti-LILRB1/2 antibodies D2M002-126 and D2M002-103HV1 reprogramed TAMs and inhibit the growth of tumor cells in the co-culture.

To determine whether TAMs can display a differential response to LPS stimulation as shown for monocytes, TAMs in another plate with the same treatment were stimulated by replacing with fresh medium supplemented with 100 ng/mL LPS for 24 hours. Cytokines of TNFα and IL10 were quantified in the culture supernatants by ELISA kits against human TNFα and IL10 (BioLegend, Cat #: 430204 and #430604). Stimulated TAMs were detached by Accutase and stained with APC anti-humanCD14, BV605 anti-human CD11B, FITC anti-human CD86, PerCP-Cy5.5 anti-human CD163, APC-Cy7 anti-human CD206, or BV785 anti-human PD-L1. The expression profile of each phenotypic marker was quantified by flow cytometry (Attune™).

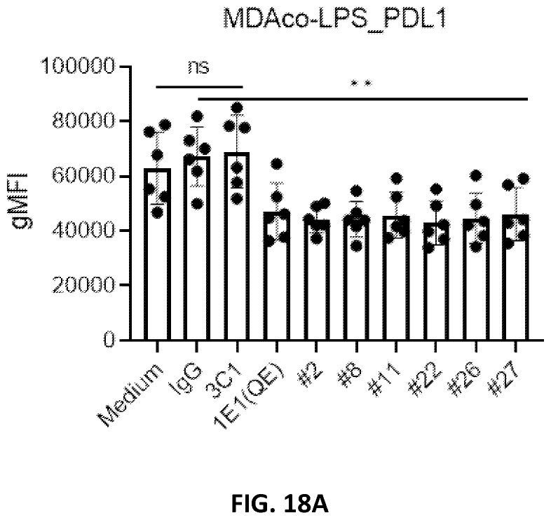

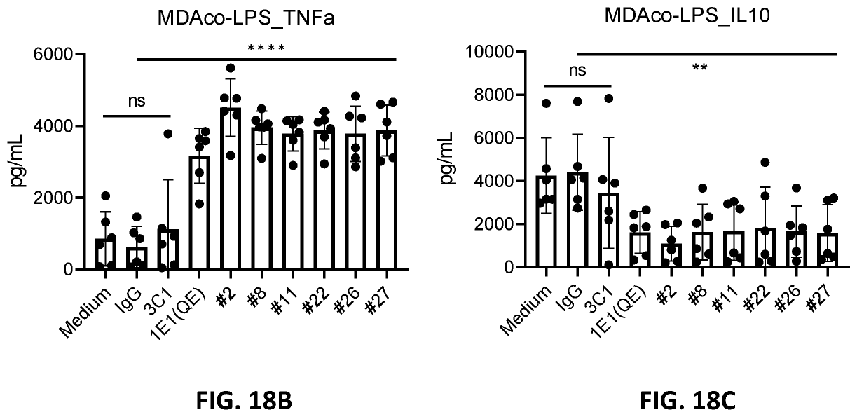

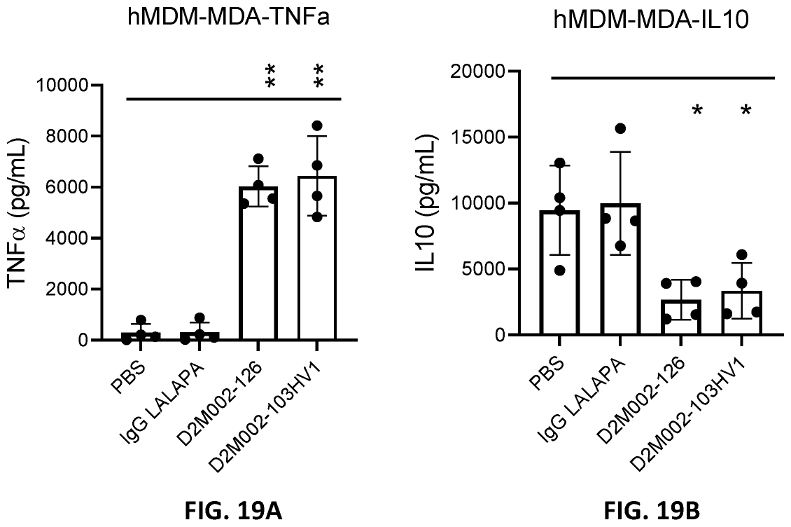

As shown in FIGS. 18A-18C, in the presence of the anti-LILRB1/2 antibodies and 1E1(QE), differentiated TAMs consistently had a less expression level of immunosuppressive marker of PD-L1, increased TNFα and decreased IL10 secretion upon LPS stimulation across multiple donors. Humanized and optimized anti-LILRB1/2 antibodies can also induce functional phenotype. As shown in FIGS. 19A-19B, in the presence of D2M002-126 or D2M002-103HV1, differentiated TAMs increased TNFα and decreased IL10 secretion upon LPS stimulation across multiple donors.

### Example 8. Anti-LILRB1/2 Antibodies Inhibited the Myeloid-Dependent Suppression of Effector T Cells

Macrophages are known as suppressors of effector T cell responses, using a variety of mechanisms including PD-L1/L2 expression, and cytokine-dependent stimulation. To test the suppressive nature of anti-LILRB1/2-matured macrophages in native adaptive T cell responses, mixed lymphocyte reactions (MLR) were performed as follows.

Human monocytes were purified from PBMCs of heathy donors by Human CD14+ Monocytes Isolation Kit. hMDMs were generated by treating human monocytes with hM-CSF (50 ng/mL) in the presence or absence of the anti-LILRB1/2 antibodies, the reference antibodies, or IgG1 isotype control (1 μg/mL) for 6 days. At the same time, immature dendritic cells (DCs) were generated from human monocytes by treatment of GM-CSF (granulocyte-macrophage colony-stimulating factor; 50 ng/mL, BioLegend, Cat #: 766106) and IL4 (10 ng/mL, BioLegend, Cat #: 766206) for 5 days. The DCs were matured by 100 ng/mL LPS stimulation for 24 hours. Allogeneic CD3+ T cells were isolated from human PBMCs of healthy voluntary donors using the Human CD3 T cells Isolation Kit according to manufacturer's instruction (BioLegend, Cat #: 480131). 100 μL mixture of mature DCs (5×10³ cells), hMDMs (2×10⁴ cells), and allotype T cells (5×10⁴ cells) were added to each well of a 96-well U-bottom plate. The cells were co-cultured in culture hood for 3 days. IFN-γ levels were quantified in the culture supernatants by the human IFN-γ ELISA kit (BioLegend, #430115).

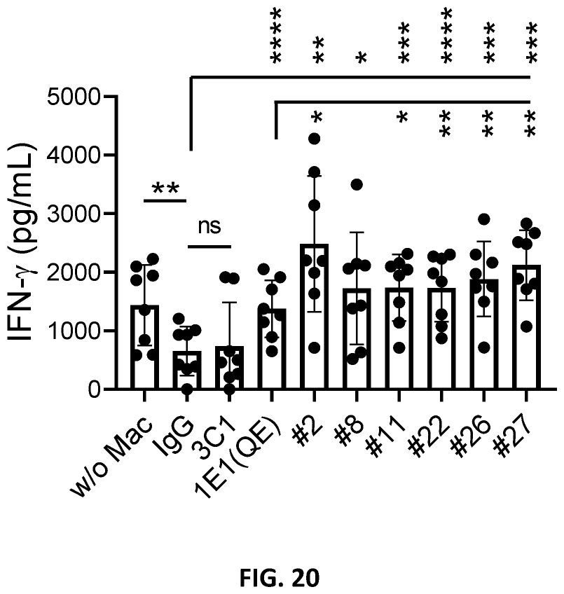

As shown in FIG. 20, as compared to MLR without hMDMs, IFN-γ secretion in MLR with differentiated hMDMs decreased, demonstrating macrophage-dependent suppression of T-cell activation. Differentiated hMDMs in the presence of the anti-LILRB1/2 antibodies or 1E1(QE), but not 3C1, enhanced the IFN-γ secretion in MLR as compared to differentiated hMDMs treated with the IgG1 isotype control, demonstrating that the anti-LILRB1/2 antibodies inhibited the myeloid-dependent suppression of T-cell activation. Moreover, the anti-LILRB1/2 antibodies showed significantly higher activity than the anti-LILRB1 antibody (3C1) in inhibiting myeloid-dependent suppression of effector T cells. The results indicate that primed human macrophages by the anti-LILRB1/2 antibodies promoted the activation of human T cells stimulated by allotype DCs.

### Example 9. Anti-LILRB1/2 Antibodies Enhanced Phagocytosis of Tumor Cells by Microphages In Vitro

LILRB1 can inhibit the phagocytosis function of macrophages as a “don't eat me” signal. Whether anti-LILRB1/2 antibodies can enhance the phagocytosis function of macrophages to tumor cells expressing HLA-G was investigated. C1R-HLAG lymphoma cells (single clone as described in Example 4) were transduced with fresh lentivirus generated from pLVX-EF1a-GFP-IRES-Puro plasmid with Lenti-X Packaging System (Takara Bio, Cat #: 631275). Clonal C1R-HLAG-GFP cell line was maintained with hygromycin and puromycin for assays. Human monocytes were purified from PBMCs of heathy donors by Human CD14+ Monocytes Isolation Kit. hMDMs were generated from monocytes in the presence of hM-CSF (50 ng/mL) for 6 days.

For direct phagocytosis assays, 25 μL hMDMs (1×10⁴ cells) were added to each well of a 96-well U-bottom plate. 25 μL of 20 μg/mL anti-LILRB1/2 antibodies, the reference antibodies 3C1 or 1E1(QE), or the IgG1 isotype control (final concentration at 5 μg/mL) were added and incubated for 30 minutes at room temperature. 50 μL equal number of C1R-HLAG-GFP tumor cells (1×10⁴ cells) were added and mixed to co-culture for 2 hours in the culture hood. hMDMs and tumor cells were detached by Accutase® and stained with APC anti-human CD14. GFP- and GFP+ CD14+ macrophages were quantified by flow cytometry (Attune™). The percentage of phagocytosis was calculated based the proportion of GFP+ macrophages in the total macrophage population.

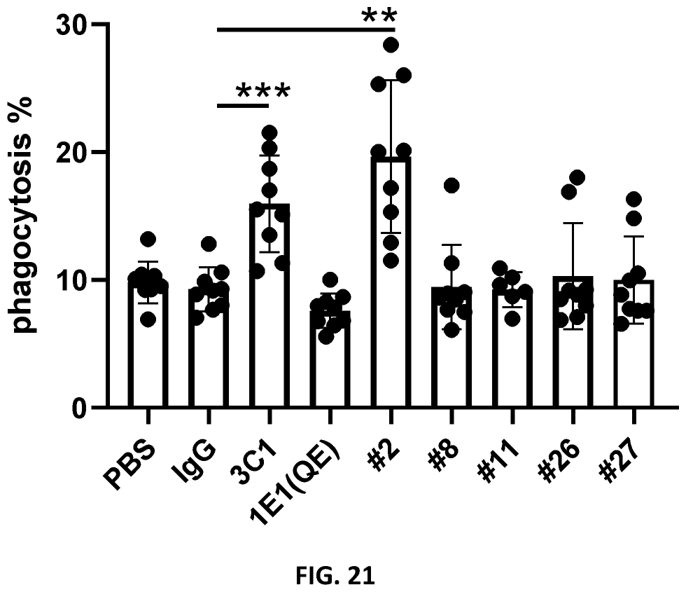

As shown in FIG. 21, one anti-LILRB1/2 antibody (clone #2) and the anti-LILRB1 antibody 3C1 enhanced the hMDMs' phagocytosis of C1R-HLAG tumor cells; whereas the anti-LILRB2 antibody 1E1(QE) and other anti-LILRB1/2 antibody clones did not enhance the phagocytosis. The results indicate that some anti-LILRB1/2 antibodies can enhance phagocytosis of the C1R lymphoma cells expressing HLA-G by macrophages.

For antibody-dependent cellular phagocytosis (ADCP) assays, C1R cells (B cells derived lymphoma expressing CD20) were used. 1 mL C1R-HLAG-GFP cells (5×10⁶ cells) were incubated with 0.1 μg/mL anti-CD20 antibody Rituximab (Bio X Cell, Cat #SIM0008) for 30 minutes at 4° C. 25 μL hMDMs (1×10⁴ cells) were added to each well in a 96-well U-bottom plate. 25 μL of 20 μg/mL anti-LILRB1/2 antibodies, the reference antibodies 3C1 or 1E1(QE), or the IgG1 isotype control (final concentration at 5 μg/mL) were added and incubated for 30 minutes at room temperature. 50 μL equal number of C1R-HLAG-GFP tumor cells (1×10⁴ cells) pre-incubated with Rituximab were added and mixed to co-culture for 2 hours in the culture hood. hMDMs and tumor cells were detached by Accutase® and stained with APC anti-human CD14. GFP− and GFP+ CD14+ macrophages were quantified by flow cytometry (Attune™). The percentage of ADCP was calculated based the proportion of GFP+ macrophages in the total macrophage population.

Anti-CD47 antibodies (“anti-CD47”) can block CD47-mediated “don't eat me” signals and therefore enhances phagocytosis, meanwhile anti-CD47 can bind CD47 on tumor cells to mediate antibody-dependent cellular phagocytosis (ADCP). To test whether anti-LILRB1/2 can enhance ADCP in the combination with anti-CD47, A375 tumor cells (human melanoma) were transfected to express GFP, 2 mL A375-GFP tumor cells (8×10⁵/mL) were incubated with 1 μg/mL or 0.1 μg/mL anti-CD47 antibody (Biolntron, Cat #: B3048) for 30 minutes at 4° C. 50 μL hMDMs (2×10⁴ cells) were added to each well in a 96-well U-bottom plate. 25 μL of 40 μg/mL anti-LILRB1/2 antibodies or the reference antibodies, or the IgG1 isotype control were added to the final concentration of 10 μg/mL and incubated for 30 minutes at room temperature. 25 μL equal number of A375-GFP tumor cells (2×10⁴ cells) pre-incubated with anti-CD47 were added and mixed to co-culture for 2 hours in the culture hood. hMDMs and tumor cells were detached by Accutase® and stained with APC anti-human CD14. GFP− and GFP+ CD14+ macrophages were quantified by flow cytometry (Attune™). The percentage of ADCP was calculated based on the proportion of GFP+ macrophages in the total macrophage population.

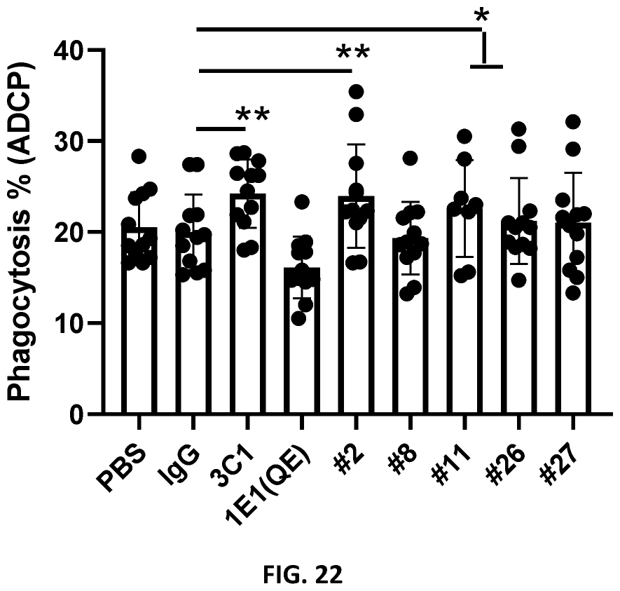

As shown in FIG. 22, some anti-LILRB1/2 antibodies (clones #2 and #26), the anti-LILRB1 antibody 3C1 enhanced the hMDMs' ADCP of C1R-HLAG tumor cells; whereas the anti-LILRB2 antibody 1E1(QE), and other anti-LILRB1/2 antibody clones did not enhance ADCP. The results indicate that some anti-LILRB1/2 antibodies can enhance the ADCP of the CIR lymphoma cells expressing HLA-G by macrophages.

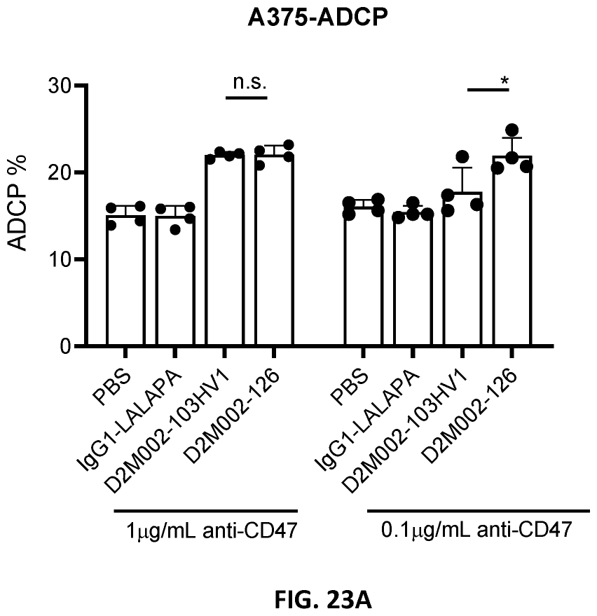

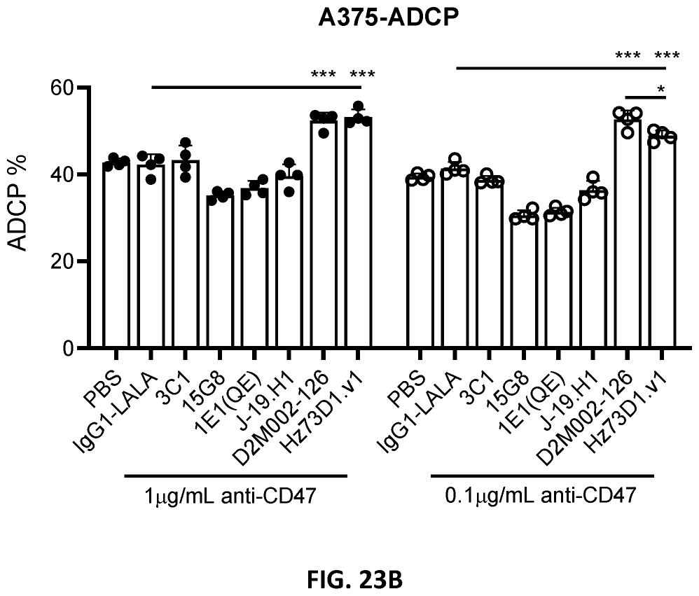

Humanized and optimized anti-LILRB1/2 can also enhance ADCP mediated by the anti-CD47. As shown in FIG. 23A, anti-LILRB1/2 antibodies of D2M002-126 and D2M002-103HV1 both enhanced the hMDMs' ADCP of A375 tumor cells. D2M002-126 may be more active than D2M002-103. As shown in FIG. 23B, only anti-LILRB1/2 D2M002-126 and reference antibody Hz73D1.v1 enhanced the hMDMs' ADCP of A375 tumor cells, whereas the anti-LILRB1 reference antibodies of 3C1 and 15G8 (heavy chain sequence: SEQ ID NO: 1060; light chain sequence: SEQ ID NO: 1059), anti-LILRB2 reference antibodies of 1E1(QE) and J-19.H1 (heavy chain sequence: SEQ ID NO: 1058; light chain sequence: SEQ ID NO: 1057) did not enhance ADCP mediated by anti-CD47. Moreover, in the presence of a low concentration of anti-CD47, e.g., at 0.1 μg/mL, D2M002-126 appeared to be more active to enhance the hMDMs' ADCP of A375 tumor cells as compared to the reference antibody Hz73D1.v1. Taken together, the anti-LILRB1/2 antibodies and anti-CD47 may synergistically enhance antibody-dependent cellular phagocytosis (ADCP) to eliminate tumor cells, D2M002-126 appeared to be more active than Hz73D1.v1 and other references.

### Example 10. Anti-LILRB1/2 Antibody D2M002-002 Lineage Recognized Monkey LILRB1 and LILRB2

CynoLILRB1 or cynoLILRB2 genes are predicted based on the sequence homology and intracellular inhibitory motifs (ITIM) to human LILRB1 and LILRB2, respectively. They were synthesized based on sequences or predicted sequences found in GenBank and cloned into expression vectors. To evaluate whether anti-LILRB1/2 antibodies can cross-react with cynomolgus (monkey) LILRB1 and LILRB2, CHO cells were transfected with pCMV3-cynoLILRB1 or pCMV3-cynoLILRB2 (Synthesized at Twist Bio) by electroporation (Neon™ Thermo Scientific, Cat #: MPK10025B). CHO-cynoLILRB1 or CHO-cynoLILRB2 cells were selected for one week and cloned by serial dilution culture. Single clone cell lines of CHO-cynoLILRB1 and CHO-cynoLILRB2 were maintained with hygromycin for assays.

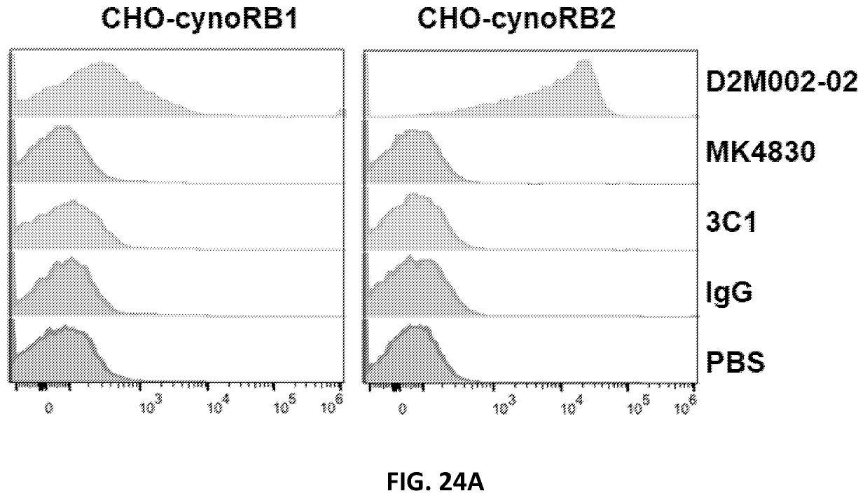

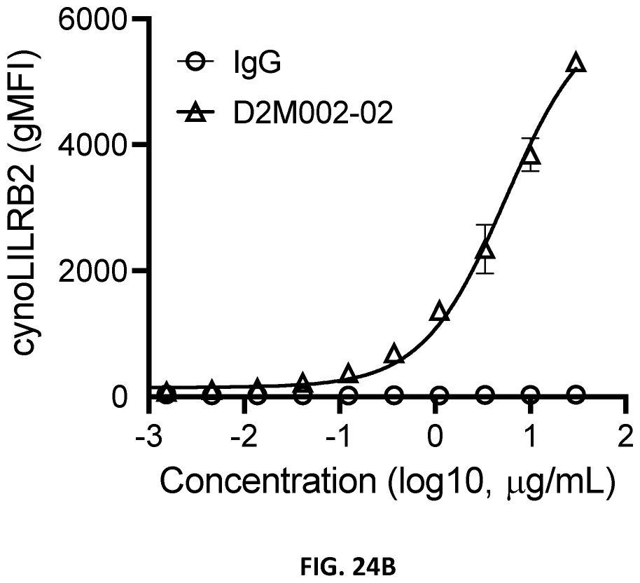

5×10⁴ CHO-cynoLILRB1 or CHO-cynoLILRB2 cells were incubated with 2 μg/mL anti-LILRB1/2 antibodies for 30 minutes, and then with APC anti-hFc (Jackson ImmunoResearch Laboratories Inc., 200×). The APC signals were quantified by flow cytometry. As shown in FIG. 24A, anti-LILRB1/2 clone #2 exhibited weak cross-reactivity with monkey LILRB1, but relatively strong cross-reactivity with monkey LILRB2. The IgG1 isotype control, and the reference antibodies 3C1 and 1E1(QE) did not bind to monkey LILRB2.

To further determine the binding affinity of anti-LILRB1/2 antibodies to monkey LILRB2, 50 μL CHO-cynoLILRB2 cells (5×10⁴ cells) were incubated with 50 μL titrated anti-LILRB1/2 antibodies at final concentrations of 30 μg/mL, 10 μg/mL, 3.33 μg/mL, 1.11 μg/mL, 0.370 μg/mL, 0.123 μg/mL, 0.041 μg/mL, 0.0137 μg/mL, 0.0046 μg/mL, 0.0015 μg/mL and 0 μg/mL at room temperature. After an incubation for 30 minutes in a 96-well U-bottom plate, the plate was centrifuged and washed with PBS 3 times. 50 μL APC anti-hFC (200×) were added to each well. After mixing for 30 minutes at room temperature, cells were washed with PBS twice. The APC signals were determined by flow cytometry (Attune™ CytPix™, ThermoFisher) within the population of live cells (viability dye DAPI negative).

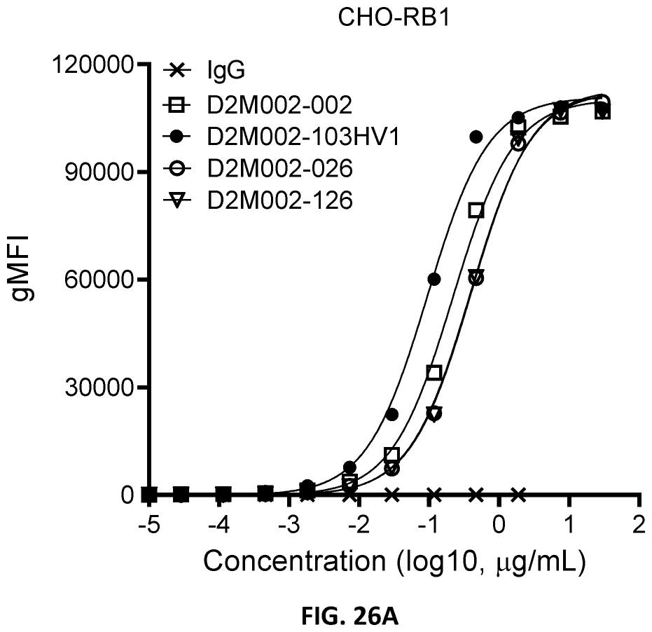

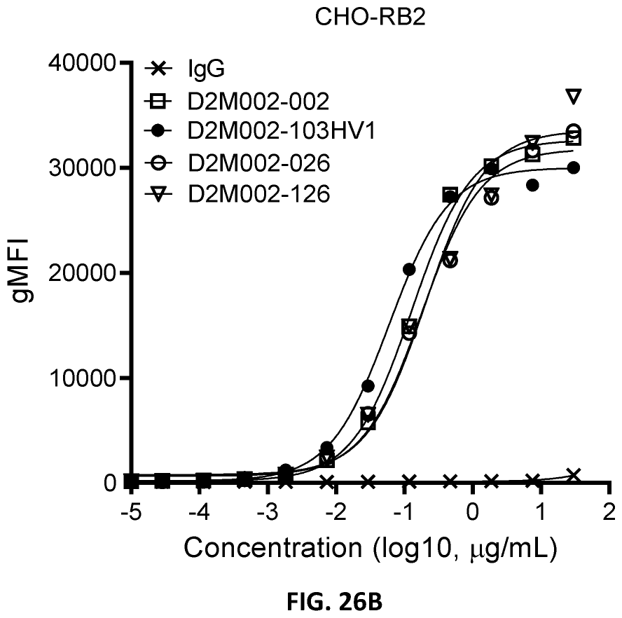

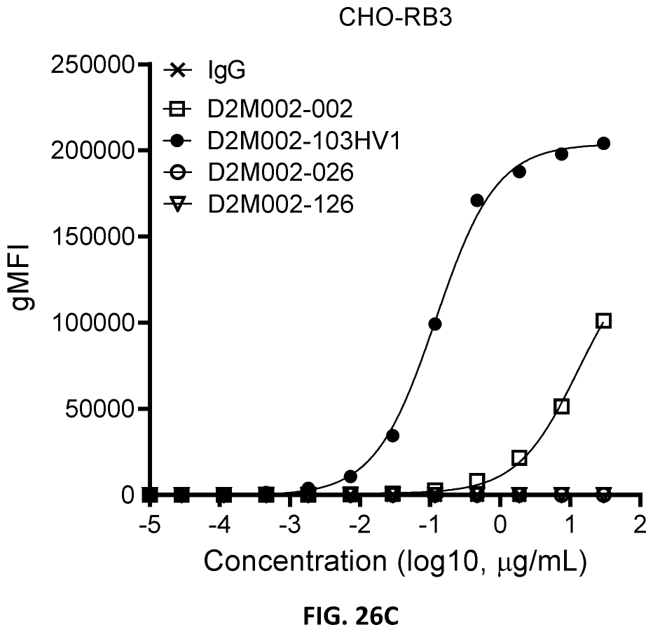

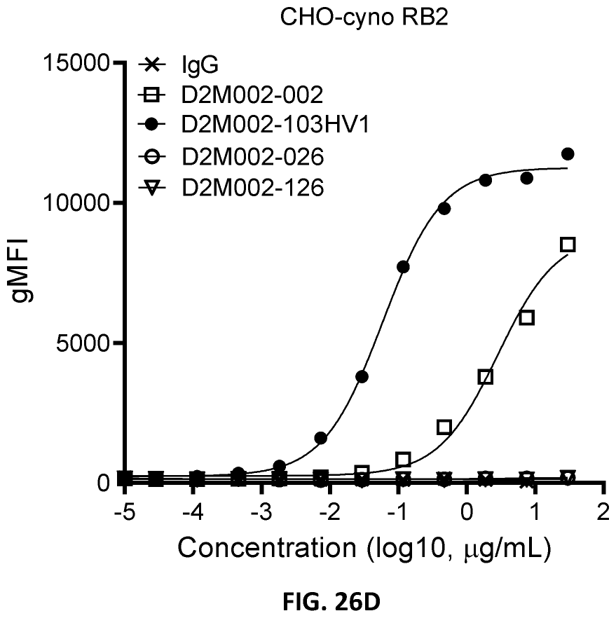

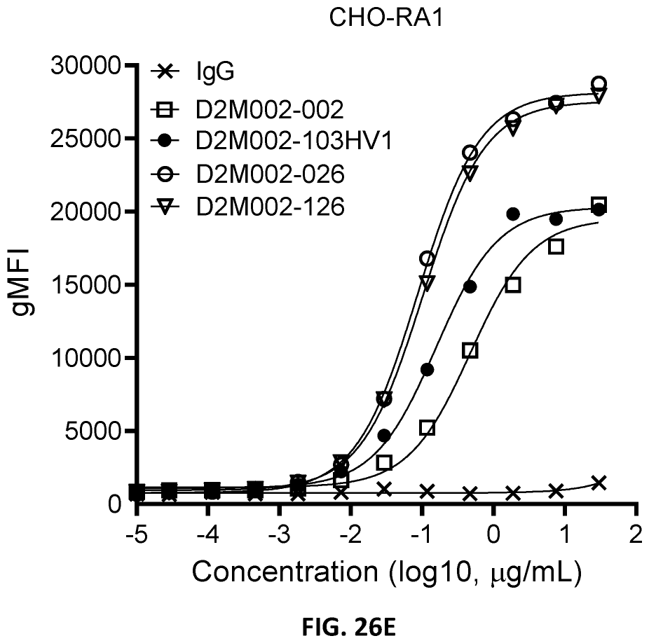

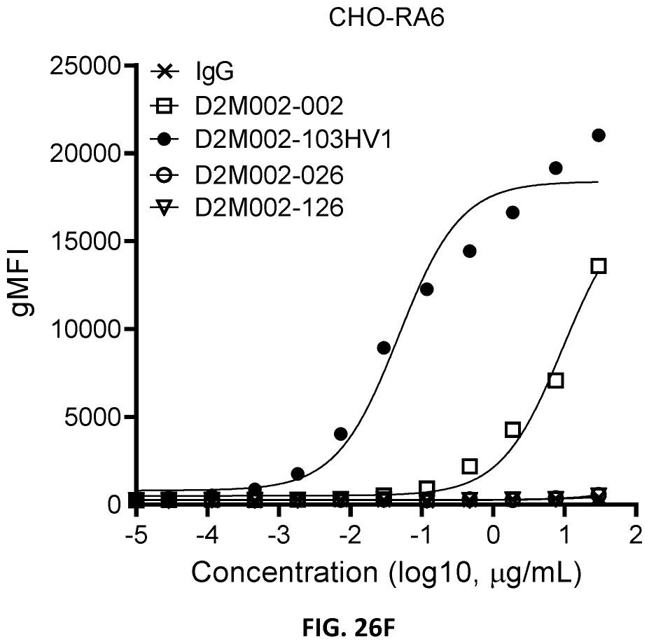

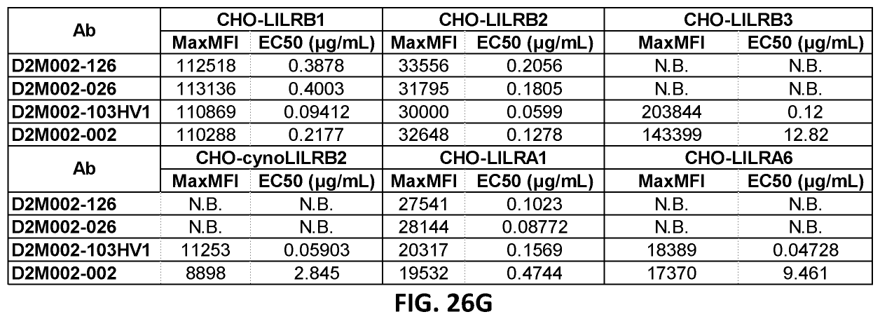

As shown in FIG. 24B and FIG. 26D, only anti-LILRB1/2 clone #2 (D2M002-002) and its humanized variant D2M002-103HV1 exhibited a relatively strong binding ability to putative monkey LILRB2 expressed in CHO cells. EC50 was also determined as 5.5 μg/mL and 0.059 μg/mL, respectively, which was calculated based on the non-linear fitting curves by GraphPad Prism software. The results indicate that some anti-LILRB1/2 antibodies can bind to cynomolgus (monkey) LILRB1 and/or LILRB2.

### Example 11. Anti-LILRB1/2 Antibodies Showed Distinct Binding Profiles to LILRA and LILRB Family Members

LILR family consists of activating LILRA receptors and inhibitory LILRB receptors. The LILRA family includes immune activating receptors of LILRA1, LILRA2, LILRA3, LILRA4, LILRA5 and LILRA6. The LILRB family includes immune suppressive receptors of LILRB1, LILRB2, LILRB3, LILRB4, LILRB5. To evaluate whether the anti-LILRB1/2 antibodies can recognize with other LILR receptors, 293T cells were transfected with pCMV3-LILRB1-GFP (SinoBiological, Cat: HG16014-ACG), pCMV3-LILRB2-GFP (SinoBiological, Cat #: HG14132-ACG), pCMV3-LILRB3-GFP(SinoBiological, Cat #: HG11978-ACG), pCMV3-LILRB4-GFP (SinoBiological, Cat #: HG16742-ACG), pCMV3-LILRB5-GFP (Sino Biological, Cat #:HG17221-ACG), pCMV3-LILRA1-GFP (SinoBiological, Cat #: HG17220-ACG), pCMV3-LILRA2-GFP (SinoBiological, Cat #: HG13273-ACG), pCMV3-LILRA3-GFP(SinoBiological, Cat #: HG13549-ACG), pCMV3-LILRA4-GFP (SinoBiological, Cat #: HG16058-ACG), pCMV3-LILRA5-GFP (SinoBiological, Cat #: HG16059-ACG) or pCMV3-LILRA6-GFP (SinoBiological, Cat #: HG29835-ACG), by Lipofectamine3000 (Thermo Scientific, Cat #L3000008). After 24 hours, 5×10⁴ transiently transfected cells (pool) were stained with the anti-LILRB1/2 antibodies (5 μg/mL), and then with APC anti-hFc as the secondary antibody. The APC signals in GFP+ cells were quantified by flow cytometry.

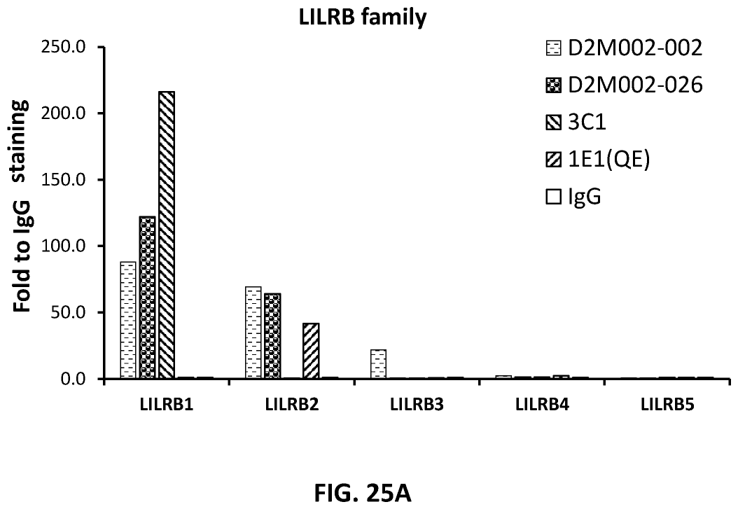

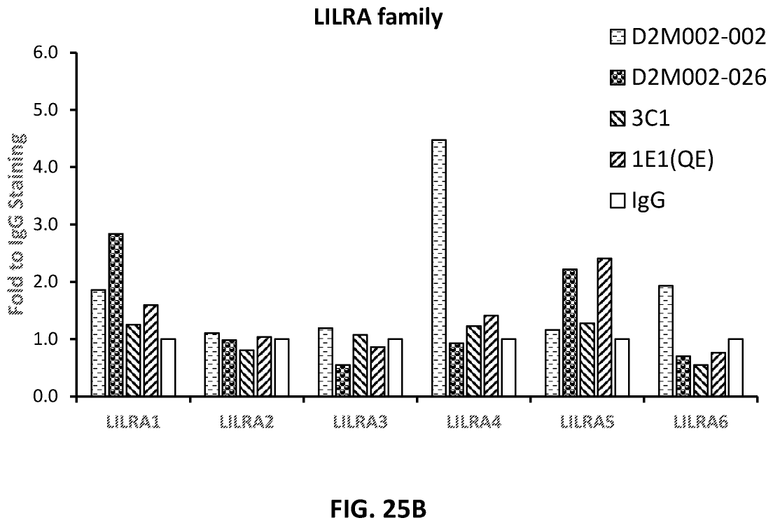

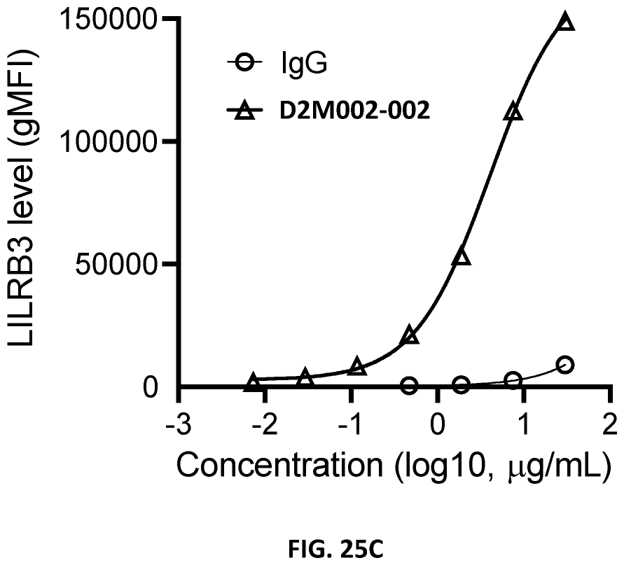

As shown in FIG. 25A, the anti-LILRB1/2 antibody D2M002-026 only binds to LILRB1 and LILRB2, whereas D2M002-002 can not only bind to LILRB1 and LILRB2, but also LILRB3. As shown in FIG. 25B, D2M002-002 can bind to LILRA1, LILRA4 and LILRA6 but no other LILRA members, whereas D2M002-026 can bind to LILRA1 and LILRA5 but no other LILRA members. Reference anti-LILRB2 1E1(QE) can bind LILRA1 and LILRA5 too.

To further determine the binding affinity of anti-LILRB1/2 antibodies to LILRB3, LILRA1 and LILRA6, CHO cells were transfected with pCMV3-LILRB3-GFP(SinoBiological, Cat #: HG11978-ACG), pCMV3-LILRA1-GFP (SinoBiological, Cat #: HG17220-ACG) or pCMV3-LILRA6-GFP (SinoBiological, Cat #: HG29835-ACG), by electroporation (Neon™ Thermo Scientific). 24 hours later, CHO-LILRB3, CHO-LILRA1 or CHO-LILRA6 cells were selected for one week and single clones were isolated and identified by serial dilution culture. Single clone cell line of CHO-LILRB3, CHO-LILRA1 or CHO-LILRA6 was maintained with hygromycin for assays. 50 μL CHO-cynoLILRB2, CHO-LILRA1 or CHO-LILRA6 cells (5×10⁴ cells) were incubated with 50 μL titrated anti-LILRB1/2 antibodies at final concentrations of 30 μg/mL, 10 μg/mL, 3.33 μg/mL, 1.11 μg/mL, 0.370 μg/mL, 0.123 μg/mL, 0.041 μg/mL, 0.0137 μg/mL, 0.0046 μg/mL, 0.0015 μg/mL and 0 μg/mL at room temperature. After an incubation for 30 minutes in a 96-well U-bottom plate, the plate was centrifuged and then washed with PBS 3 times. 50 μL APC anti-hFC (200×) were added to each well. After mixing for 30 minutes at room temperature, cells were washed with PBS twice. The APC signals were determined by flow cytometry (Attune™ CytPix™, ThermoFisher) within the population of live cells (viability dye DAPI negative).

As shown in FIG. 25C, the anti-LILRB1/2 antibody D2M002-002 exhibited a strong binding ability to LILRB3 expressed in CHO cells. EC50 was also determined as 4 μg/mL or 26.6 nM, which was calculated based on the non-linear fitting curves by GraphPad Prism software.

Humanized and optimized anti-LILRB1/2 antibodies also showed distinct binding profiles to LILRA/B family members. As shown in FIGS. 26A-26B, and 26E, the anti-LILRB1/2 antibodies, D2M002-126 and its parental D2M002-026, D2M002-103HV1 and its parental D2M002-002, exhibited a strong binding ability to LILRB1, LILRB2 and LILRA1 expressed in CHO cells. As shown in FIGS. 26C-26D, and 26F, only D2M002-103HV1 and its parental D2M002-002, exhibited binding ability to human LILRB3, human LILRA6 and putative monkey LILRB2 (cynoRB2) expressed in CHO cells. As shown in FIG. 26G, EC50 values were also determined based on the non-linear fitting curves by GraphPad Prism software. Humanization and optimization have improved the binding of D2M002-002 variants to LILRB3, LILRA6 and putative cyno LILRB2 significantly compared to parental D2M002-002. The results confirmed D2M002-126 does not bind putative cyno LILRB2.

### Example 12. Humanized Anti-LILRB1/2 Antibodies Blocked LILRB2 Binding to Reported Putative Ligands

LILRB2 has been reported to bind multiple potential ligands to activate LILRB2 signaling, including SEMA4A, MAG, CD1C, CD1D and ANGPTL2 and ANGPTL5. To examine whether anti-LILRB1/2 antibody blocks the interactions of LILRB2 and its potential ligands, ELISA-based assays were developed. The 96-well assay plates were coated with 100 μL ligand proteins (3 μg/mL) of SEMA4A (SinoBiological, Cat #: 11756-H08H), MAG (SinoBiological, Cat #: 13186-H08H), CD1C (SinoBiological, Cat #: CT057-C08H), CD1D (SinoBiological, Cat #: CT042-H08H), ANGPTL2 (SinoBiological, Cat #: 11014-H07H) or ANGPTL5 (SinoBiological Cat #: 11094-H09B) overnight at 4° C. After two washes with PBS, the plate was blocked with 1% BSA for 1 hour at room temperature. LILRB2-ECD-His (SinoBiological, Cat #: 14132-H08H-B) at the final concentration of 5 μg/mL were preincubated with titrated anti-LILRB1/2 antibody D2M002-126 or isotype control (IgG) at the final concentrations of 15 μg/mL, 7.5 μg/mL, 3.75 μg/mL, 1.875 μg/mL, 0.938 μg/mL, 0.469 μg/mL, 0.235 μg/mL, and 0 μg/mL at room temperature for 30 minutes.

The preincubated mixtures were added into wells and incubated for 2 hours at room temperature on a plate shaker. The plates were washed twice with plate washer and 100 μL Streptavidin-HRP (1:200, R&D Systems, Cat #: 890803) were added into each well and incubated for 1 hour at room temperature on a plate shaker. The plates were washed twice with plate washer and developed with TMB substrate to detect the binding signals with a multimode plate reader (Varioskan™ Lux, Thermo Scientific).

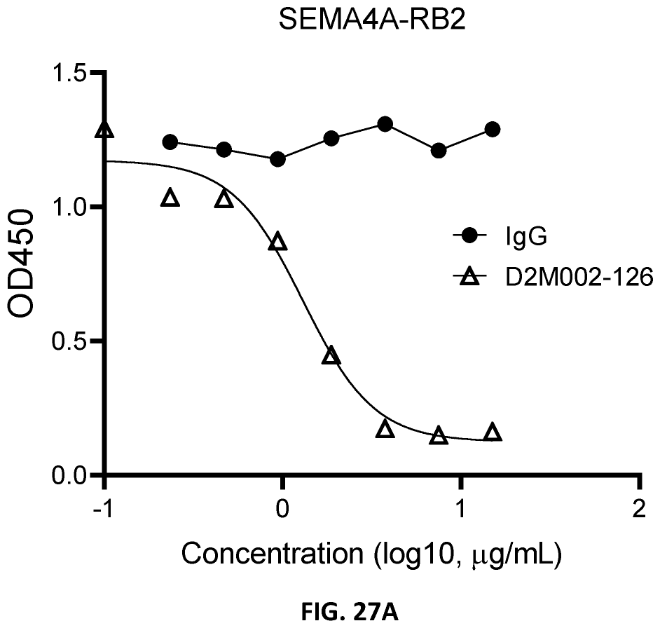

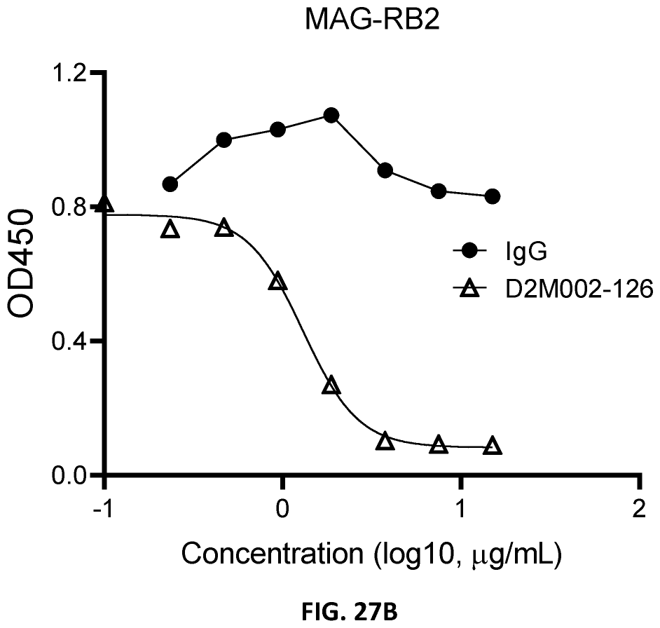

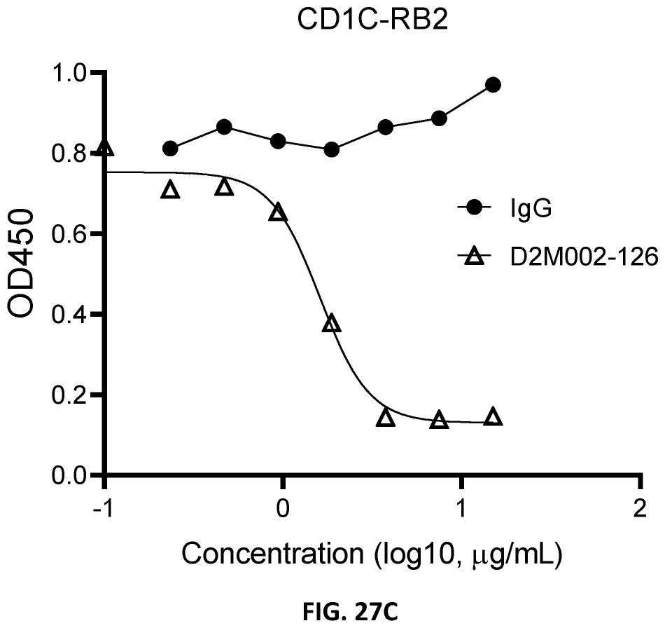

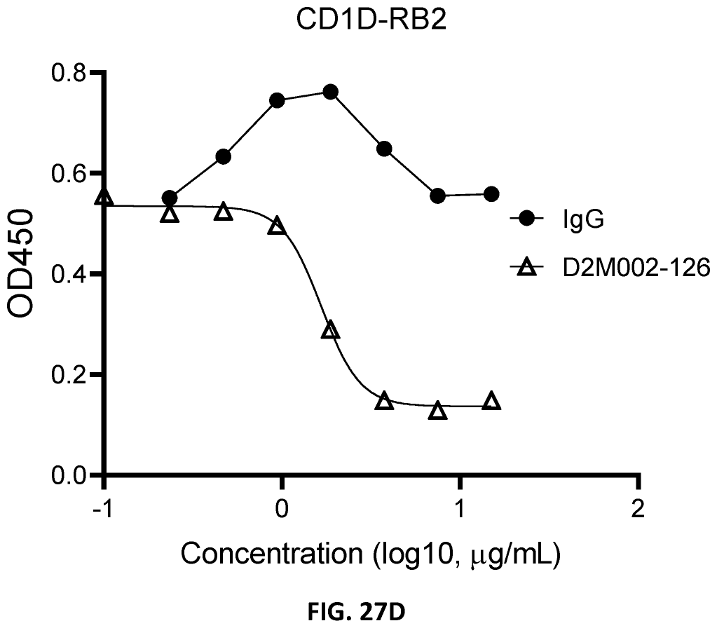

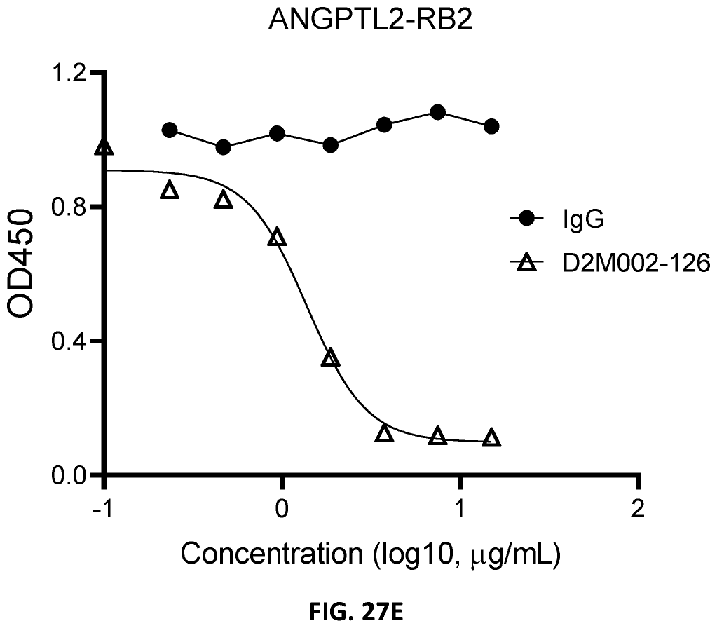

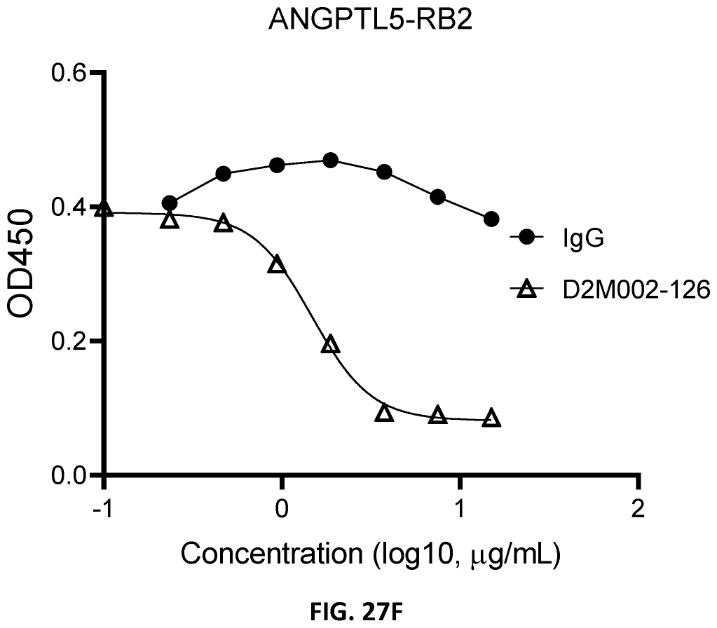

As shown in FIGS. 27A-27F, D2M002-126 demonstrated the capability to block the interactions of LILRB2 with putative ligands SEMA4A (FIG. 27A), MAG (FIG. 27B), CD1C (FIG. 27C), CD1D (FIG. 27D), ANGPTL2 (FIG. 27E) and ANGPTL5 (FIG. 27F). D2M002-126 can efficiently block the interaction of LILRB2 with HLA-G and other putative ligands, therefore, can maximally release the immunosuppression mediated by LILRB2.

### Example 13. Humanized Anti-LILRB1/2 Antibodies Enhanced the Cytotoxicity of Human NK Cells Against Tumor Cells

LILRB1 expresses in human NK cells and serves as an immunosuppressive receptor through interaction with the ligand HLA-G. Whether anti-LILRB1/2 antibodies can enhance the cytotoxicity function of human NK cells to tumor cells expressing HLA-G was investigated. Human primary NK cells were isolated from heathy PBMC and activated overnight in the presence of 500 IU/mL rhIL2 (BioLegend). The NK cells were transduced with the lentiviral particles expressing LILRB1 by spin infection. 48 hours post transduction, 50 μL transduced NK cells (5,000/well) were cocultured with 50 μL A253-GFP tumor cells equally in the presence of 10 μg/mL D2M002-126, the reference antibody Hz73D1.v1, or the isotype control IgG1-LALA or PBS for 4 hours in the culture hood. The cytotoxicity (killing) of NK cells was quantified by flow cytometry based on the proportion of DAPI+GFP+ cells in total GFP+ cells.

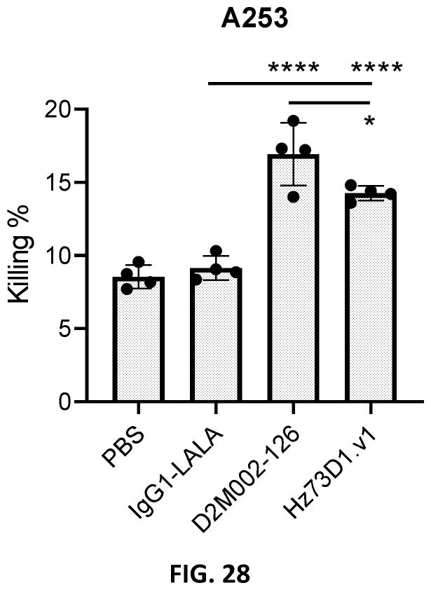

The results showed that D2M002-126 enhanced NK function. As shown in FIG. 28, the anti-LILRB1/2 antibody D2M002-126 and reference antibody Hz73D1.v1 enhanced the cytotoxicity of NK cells against A253 tumor cells. Representative NK data from multiple donors and independent experiments are shown. Moreover, D2M002-126 induced significantly higher NK killing as compared with reference antibody Hz73D1.v1.

### Example 14. Humanized Anti-LILRB1/2 Antibodies Enhanced the Cytotoxicity of Human CD8+ T Cells Against Tumor Cells

LILRB1 expresses in human CD8+ T cells and serves as an immunosuppressive receptor through interaction with the ligand HLA-G. Whether anti-LILRB1/2 antibodies can enhance the cytotoxicity function of human CD8+ T cells to tumor cells expressing HLA-G was investigated. Human primary CD8+ T cells were isolated from healthy donor's PBMC and activated for 24 hours in the presence of anti-CD3/CD28/CD2 beads (STEMCELL) and 100 IU/mL rhIL2 (BioLegend). The activated CD8+ T cells were transduced with the lentiviral particles expressing LILRB1 by spin infection. 48 hours post transduction, 50 μL transduced CD8+ T cells (2×10⁴/well) were cocultured with 50 μL C1R-GFP tumor cells expressing both anti-CD3 scFv (OKT3) and HLA-G (1×10⁴/well) in the presence of 10 μg/mL D2M002-126, reference antibodies of 3C1, 15G8 and Hz73D1.v1, or the isotype control IgG1-LALA for 4 hours in the culture hood. The cytotoxicity (killing) of CD8+ T cells were quantified by flow cytometry based on the proportion of DAPI+GFP+ cells in total GFP+ cells.

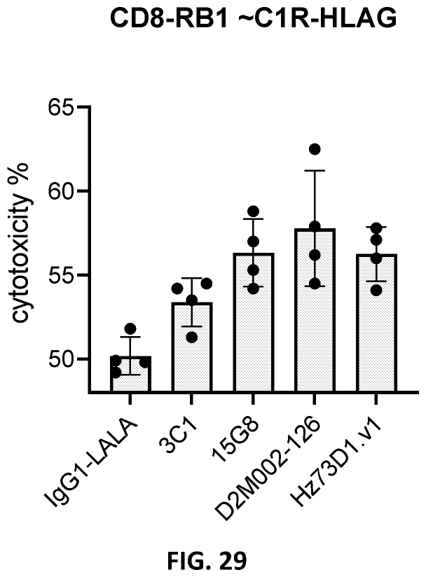

The results showed that D2M002-126 enhanced cytotoxicity function of CD8+ T cells. As shown in FIG. 29, the anti-LILRB1 and anti-LILRB1/2 antibodies enhanced the cytotoxicity of CD8+ T cells against CIR tumor cells. Representative data from multiple donors and independent experiments are shown. Moreover, D2M002-126 appeared to induce higher cytotoxicity on CD8+ T cells as compared with anti-LILRB1 reference antibodies 3C1 and 15G8, and anti-LILRB1/2 reference antibody Hz73D1.v1.

### Example 15: D2M002-126 Exhibited Stability in Human Plasma In Vitro

To further investigate the serum stabilities of D2M002-126, 200 μg D2M002-126 (1 mg/mL) was mixed with equal volume of human fresh plasma sampled from a healthy volunteer or PBS and incubated in a 37° C. water bath for a total of 3 weeks. 100 μL stressed antibody were aliquoted at week 0, week 1, week 2 and week 3 and stored at −20° C. Cell-based antibody binding affinities were measured by incubating titrated antibodies from three treatment groups of D2M002-126 with either PBS or plasma (week 0, week 1, week 2 and week 3) as well as the stock antibody with engineered CHO-LILRB1 or CHO-LILRB2 cells. 50 μL CHO-LILRB1 or CHO-LILRB2 (4×10⁴ cells) were added to each well of a 96-well U-bottom culture plate. 50 μL titrated D2M002-126 were added to final concentrations of 30 μg/mL, 7.5 μg/mL, 1.875 μg/mL, 0.469 μg/mL, 0.117 μg/mL, 0.0293 μg/mL, 0.0073 μg/mL, 0.0018 μg/mL, 0.000458 μg/mL, 0.000114 μg/mL, 0.00003 μg/mL and 0 μg/mL and incubated for 30 minutes at room temperature. The plate was centrifuged, and cells were washed with PBS three times. 50 μL Alexa Fluor® 647 Affini Pure F(ab′)₂ Fragment Goat Anti-Human IgG, Fcγ fragment specific (Jackson ImmunoResearch Laboratories Inc., Cat #: 109-606-098) was added to each well of the plate. After incubation for 30 minutes at room temperature, cells were washed with PBS twice. Signals of APC were determined by flow cytometry (Attune™ CytPix™, ThermoFisher) within the population of live cells (viability dye DAPI negative).

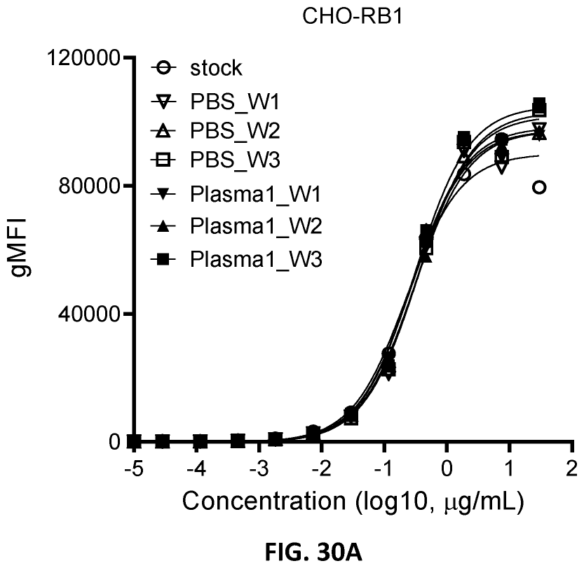

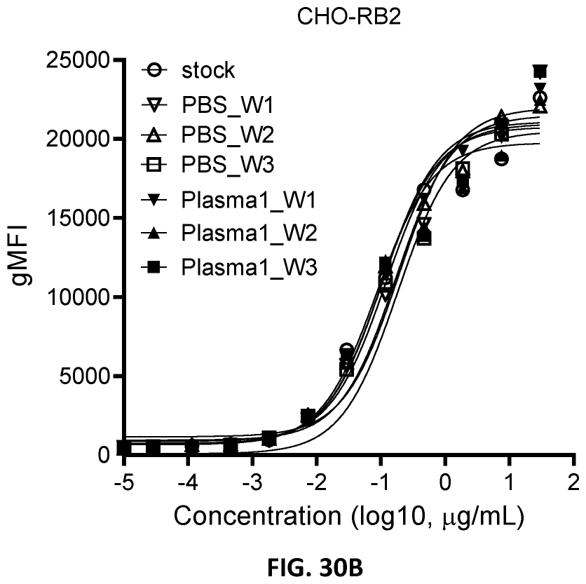

D2M002-126 has exhibited serum stability in fresh human plasma in vitro. As shown in FIGS. 30A-30B, D2M002-126 incubated in human plasma or PBS at 37° C. for 1 or 2 or 3 weeks exhibited strong binding affinities to CHO-LILRB1 (FIG. 30A) or CHO-LILRB2 (FIG. 30B) with similar EC50 values before and post human plasma or PBS incubation. The EC50 values were calculated based on non-linear fitting curves by GraphPad Prism software.

### Example 16: D2M002-126 Exhibited Long-Term Stability in Mice

To further investigate the stability of D2M002-126 in vivo, 200 μg D2M002-126 (10 mg/kg) was injected retro-orbitally into C57BL/6 mice (B6 mice). For each animal, 50 μL of blood was collected at timepoints of 2 hours, 1 day, 3 days, 6 days, 8 days and 10 days post-injection by retro-orbital bleeding. To detect the concentration of anti-LILRB1/2 in the plasma, a sandwich ELISA was developed. A 96-well assay plate was coated with 100 μl goat anti-human IgG, F(ab′)₂ specific (1 μg/mL, Jackson ImmunoResearch, Cat #: 109-005-097) overnight at 4° C. After two washes with PBS, the plate was blocked with 1% BSA for 1 hour at room temperature. After two additional PBS washes, 100 μL diluted plasma samples (30,000× dilutions) and titrated standards (D2M002-126) were added and incubated for 2 hours at room temperature. Afterwards, the plate was washed with PBS twice and 100 μL HRP anti-human hFc (1:10000, Jackson ImmunoResearch, Cat #: 109-035-008) were added and developed with TMB to detect the binding signals. The concentrations of plasma D2M002-126 were calculated based on the standards used for each assay.

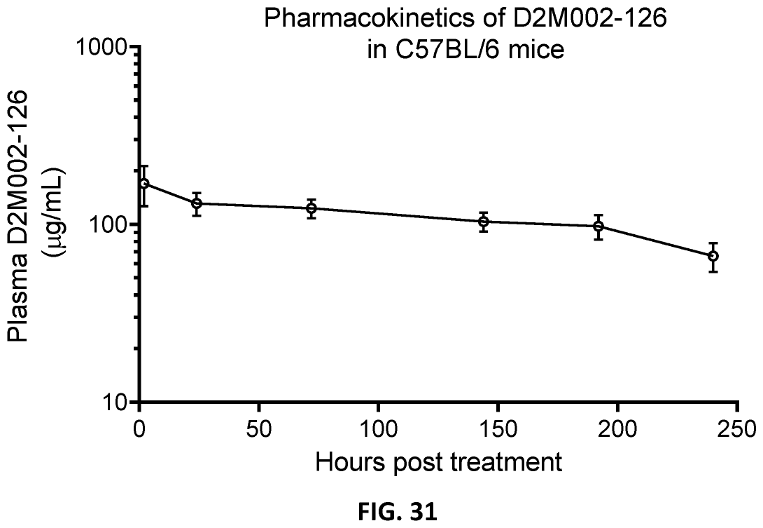

D2M002-126 has shown in vivo stability in mice. As shown in FIG. 31, about 50% of D2M002-126 persisted in vivo through 10 days post-injection, demonstrating that D2M002-126 has long-term stability in mice in vivo.

### Example 17: D2M002-126 Prevented the A375 Tumor Growth in the A375/hMDM Compliant Tumor Model

To investigate the ability of D2M002-126 to prevent tumor growth in vivo, 50 μL 1.0×10⁶ A375 cells were mixed with 50 μL 2.0×10⁵ fresh hMDM. The 100 μL mixed cells were implanted subcutaneously in the left flank of 8-week old NCG mice (Charles River). The mice were grouped and dosed i.p. with D2M002-126 or IgG-1-LALA-PA (10 mg/kg) every other day (3 doses per week) in the first two weeks from day 0 post tumor inoculation. Tumor sizes were measured twice per week from day 9 till day 25 post tumor inoculation by a digital caliper.

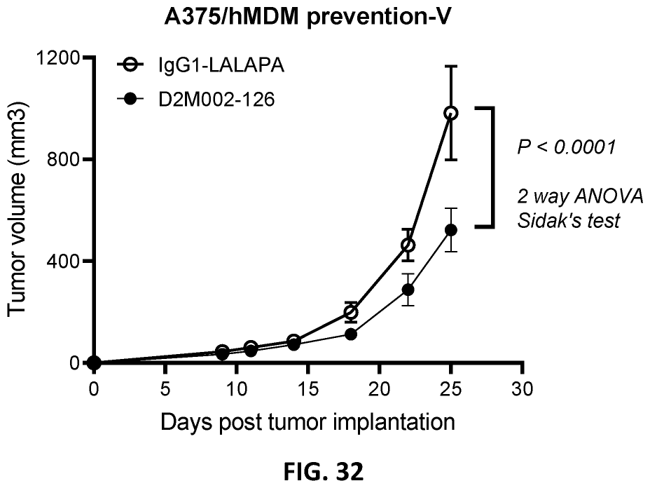

The results showed that D2M002-126 can reprogram hMDM to prevent A375 growth in NSG mouse model. As shown in FIG. 32, D2M001-126 significantly prevented the tumor growth of A375 (about 55% TGI) in mice (8-12 mice per group) relative to isotype IgG1 control group.

### Example 18: D2M002-126 Inhibited the A375 Tumor Growth Through hDMDs in the A375 Tumor Model

To investigate the ability of D2M002-126 to inhibit tumor growth through hMDMs in vivo, 100 μL 1.0×10⁶ A375 cells were implanted subcutaneously in the left flank of 8-week old NCG mice (Charles River). On day 9 post tumor inoculation and tumor sizes reach to about 30 mm³, the mice were grouped and 10 μL fresh hMDMs pre-incubated with 10 μg/mL D2M002-126 or IgG-1-LALA-PA were injected intratumorally. The mice were dosed i.p. with 10 mg/kg D2M002-126 or IgG-1-LALA-PA twice per week in the first two weeks from day 9 post tumor inoculation. Tumor sizes were measured twice per week from day 9 till day 25 post tumor inoculation by a digital caliper.

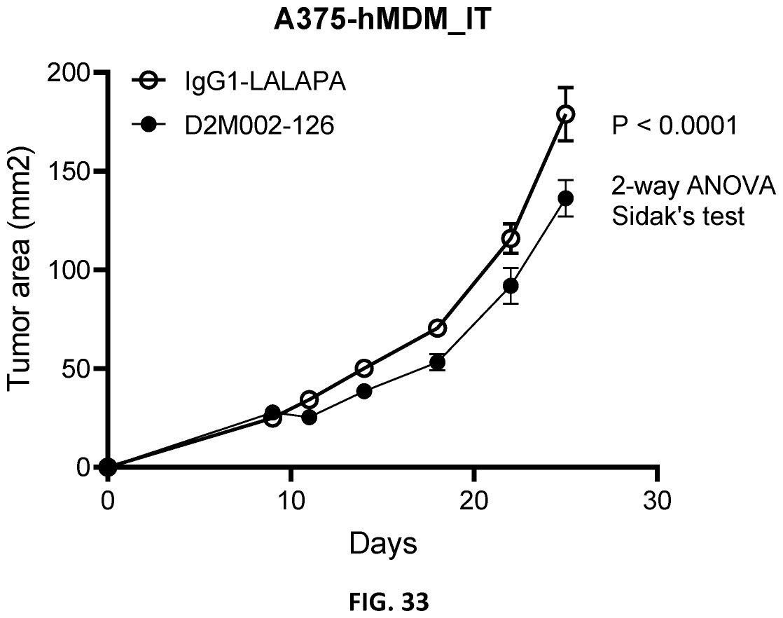

The results showed that D2M002-126 can reprogram hMDM to inhibit A375 growth in NSG mouse model. As shown in FIG. 33, D2M001-126 significantly inhibited the tumor growth of A375 (about 42% TGI) in mice (8-12 mice per group) relative to isotype IgG1 control group.

### Example 19: Humanized Anti-LILRB1/2 Antibody Inhibited the A375 Tumor Growth in the A375 Tumor huPBMC Model

To investigate the ability of D2M002-126 to inhibit tumor growth in vivo, on day −7 before tumor inoculation, 1×10⁷ human PBMC were i.v. injected into 8-week old NCG mice (Charles River). On day 0, 100 μL 5.0×10⁶ A375 cells were implanted subcutaneously in the left flank of huPBMC NCG mice. The mice were randomly grouped and dosed i.p. with 10 mg/kg D2M002-126 or IgG1-LALA isotype control twice per week for total 4 weeks. Tumor sizes were measured twice per week from day 7 till day 38 post tumor inoculation by a digital caliper.

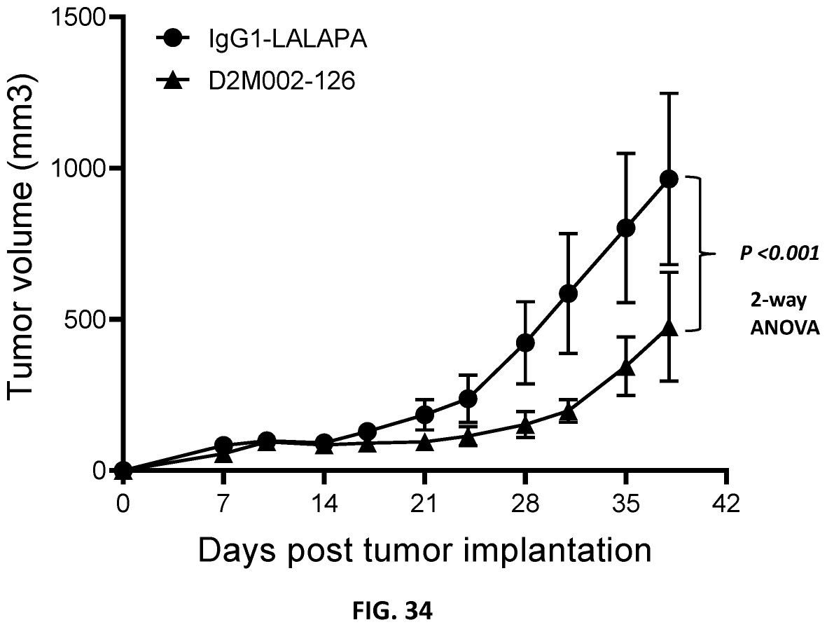

The results showed that D2M002-126 can inhibit A375 growth in human PBMC reconstituted NCG mouse model. As shown in FIG. 34, D2M001-126 significantly inhibited the tumor growth of A375 (about 50% TGI) in mice (8-12 mice per group) relative to isotype IgG1 control group.

### Example 20: Humanized Anti-LILRB1/2 Antibodies had Distinct Binding Epitopes Comparing to Reference Antibodies, as Determined by Epitope Binning Analysis

Epitope binning was carried out to characterize the binding of different anti-LILRB1, anti-LILRB2 or anti-LILRB1/2 to LILRB1 or LILRB2 protein pair-wisely against all mAbs in a set to assess whether they block one another's binding to a specific site of the antigen or not. The mAbs that block binding to the same epitope are competitive and “binned” together. To examine the binding epitopes of anti-LILRB2 antibodies and reference antibodies, the binning analysis was performed using Gator Prime BLI (Biolayer Interferometry) system (Gator Bio, USA). Specifically, the anti-LILRB1/2 antibodies were loaded to HFC (Anti-hIgG Fc, Gator Bio, Cat #: 160003). Then, the probes were incubated with corresponding monomeric antigens for 3-4 minutes, at 25° C. in K buffer (Gator Bio, Cat #: 120011). The dissociation in the other competitive antibodies was monitored for 5 or 10 minutes. The probes were regenerated between binding cycles with Regeneration Buffer (Gator Bio, Cat #: 120012). Binding kinetics was analyzed using the software supplied by the manufacturer.

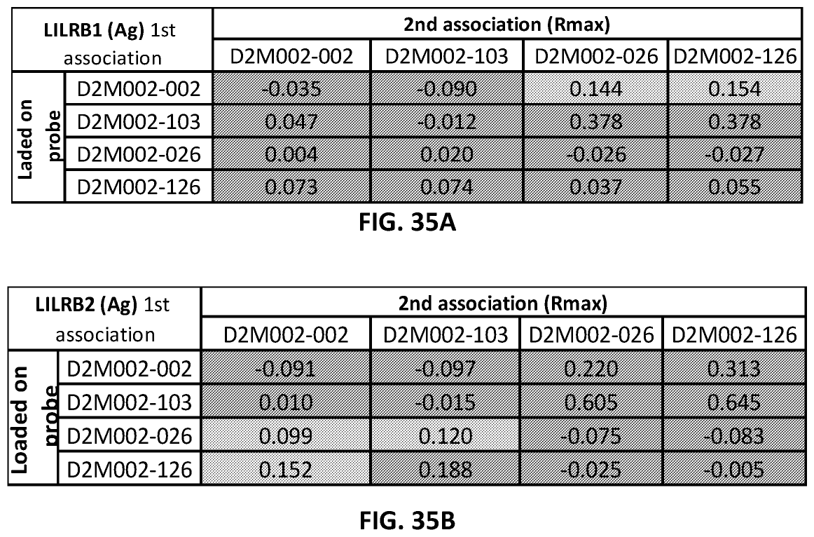

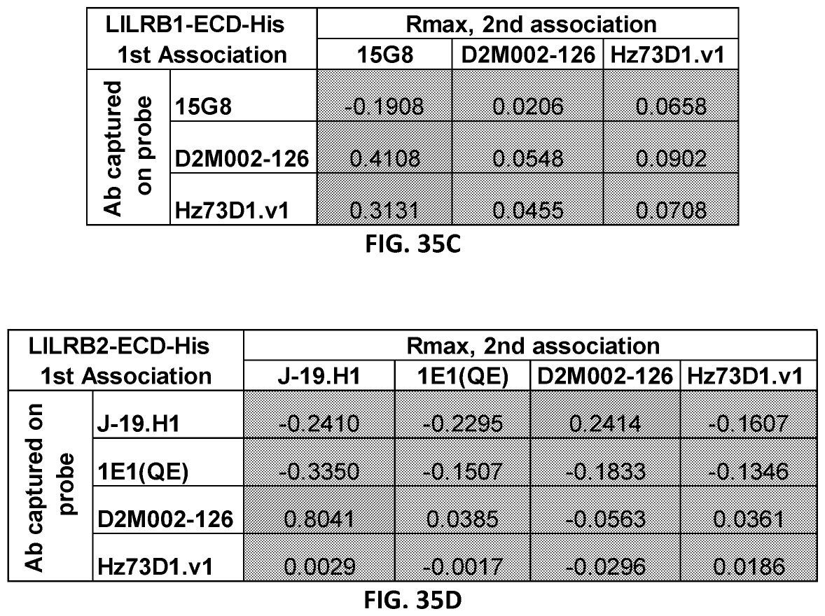

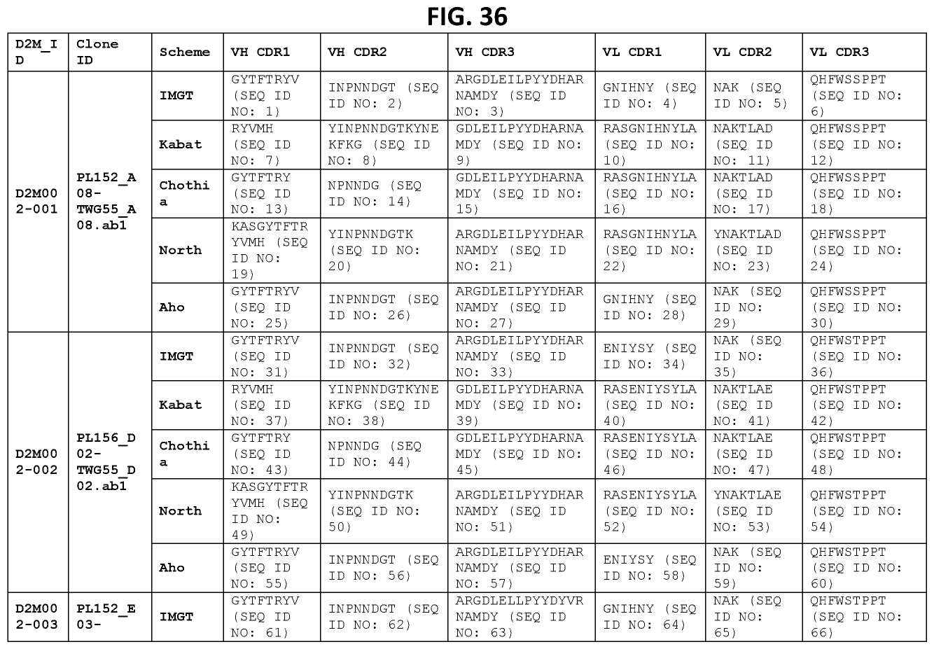

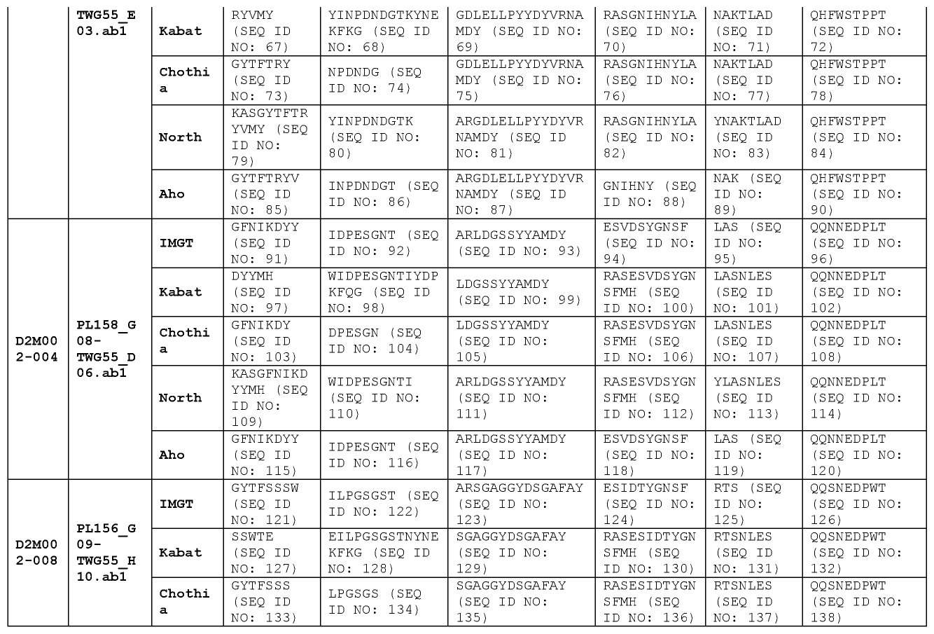

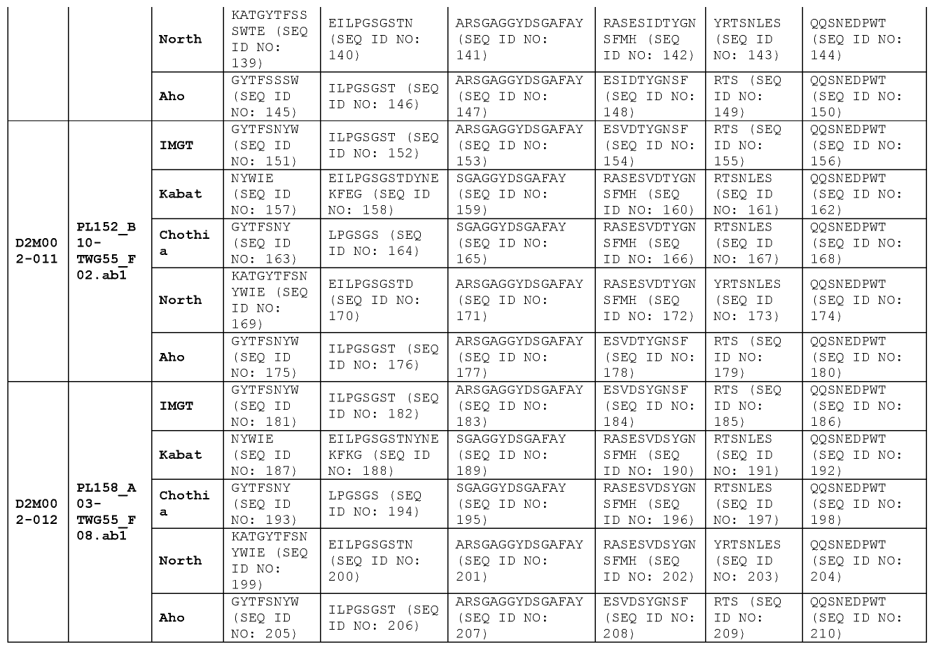

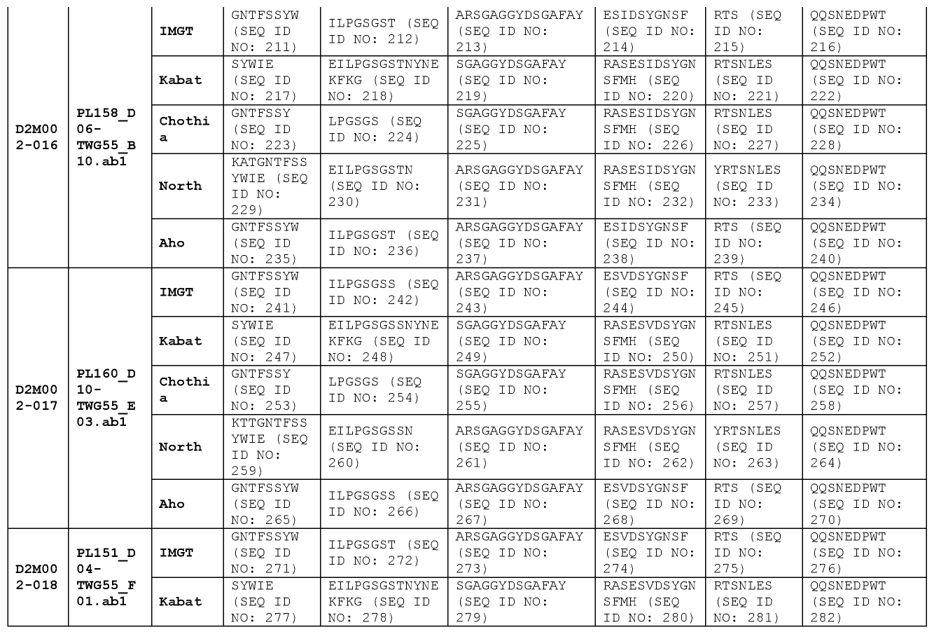

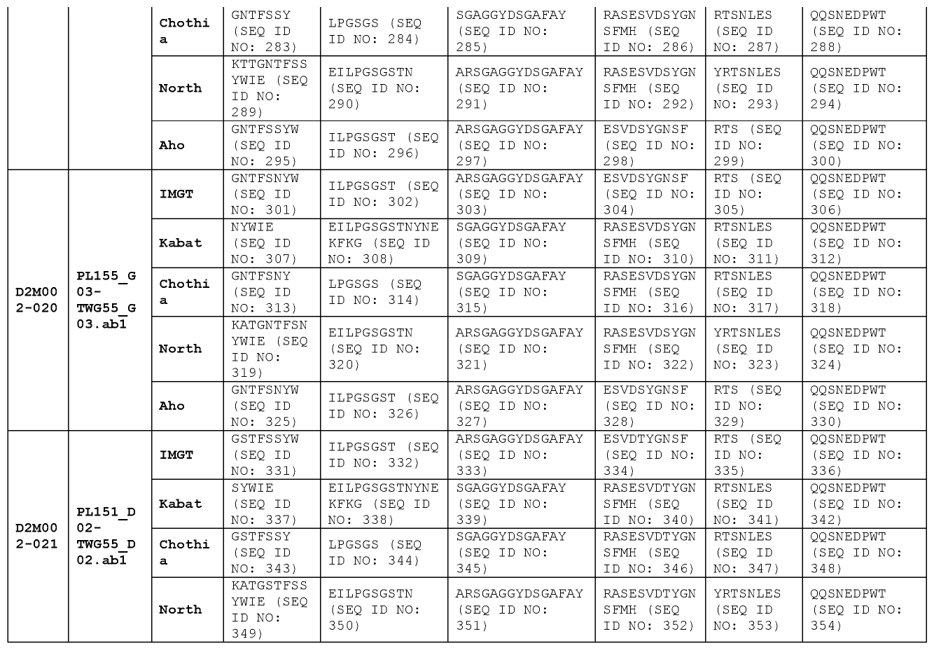

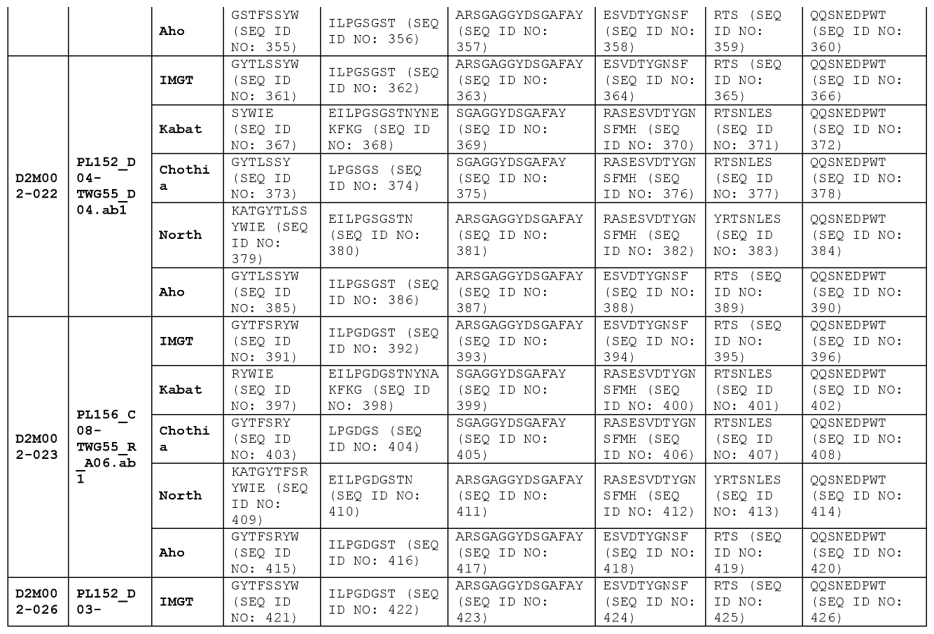

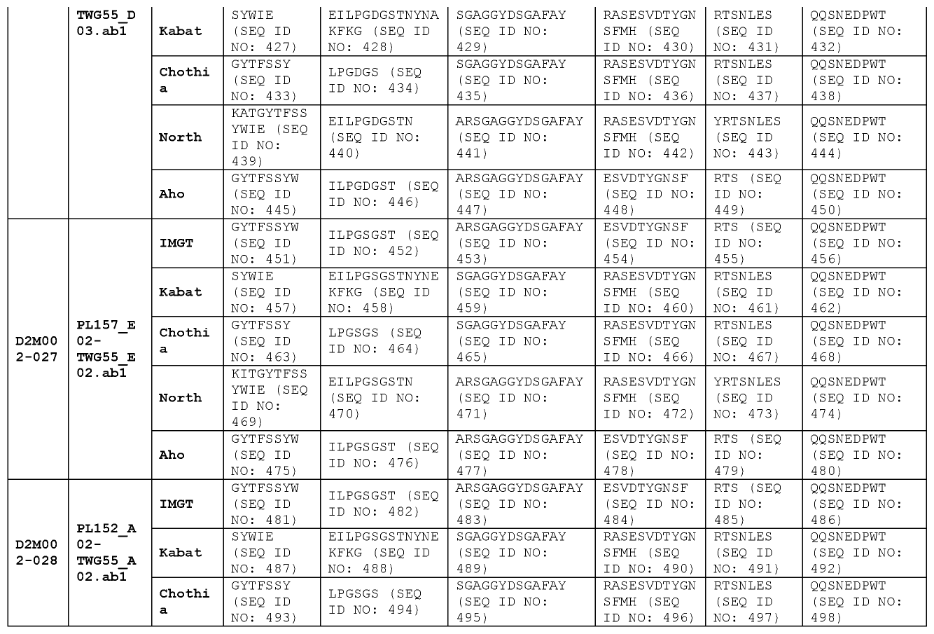

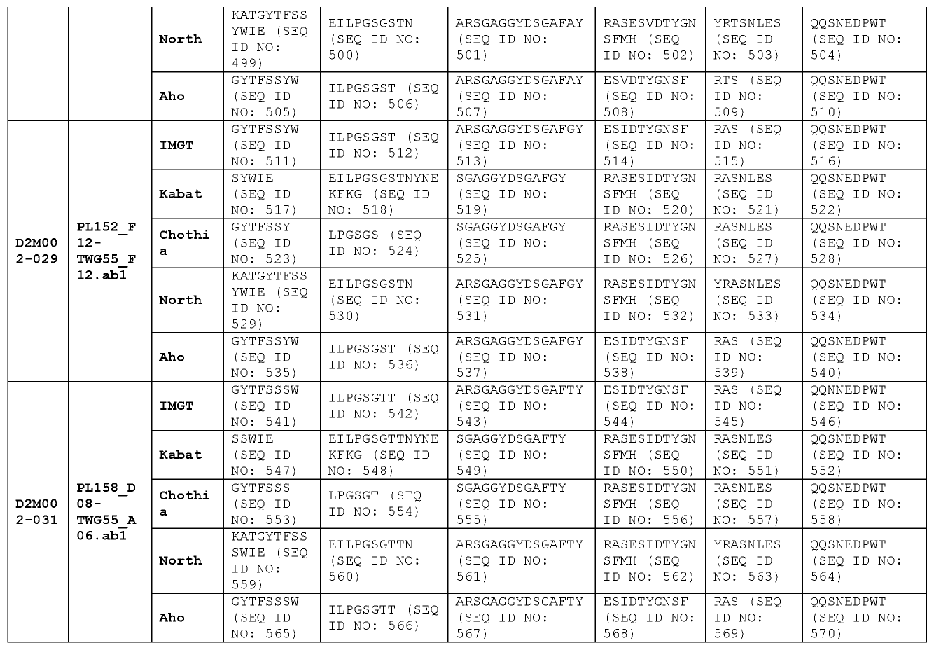

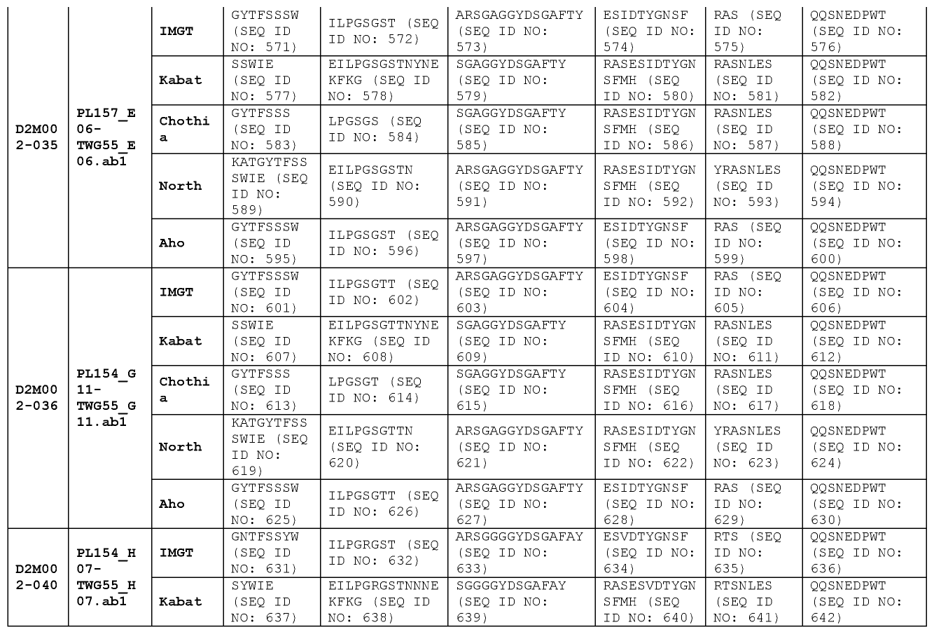

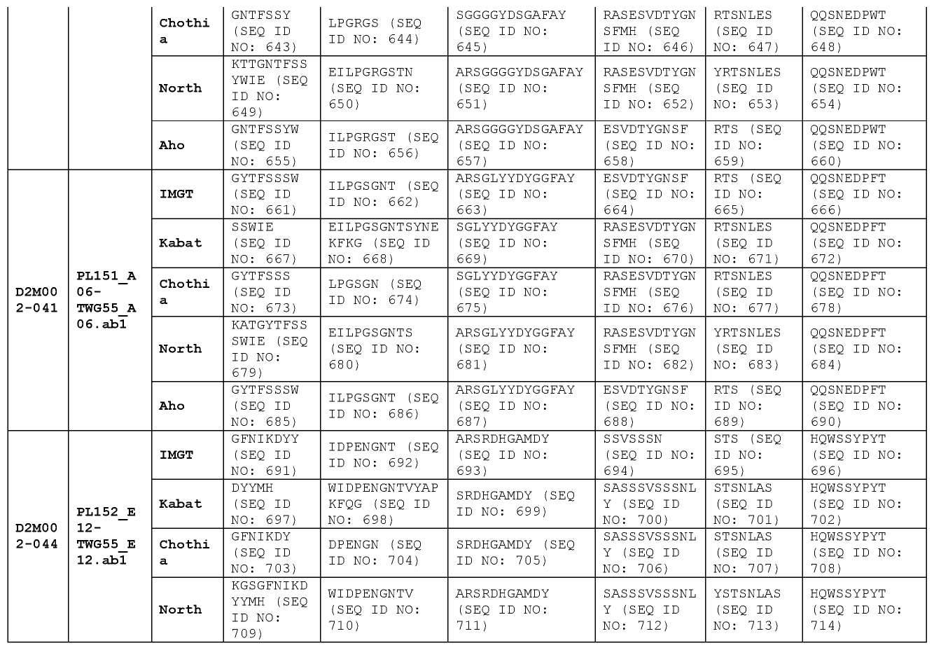

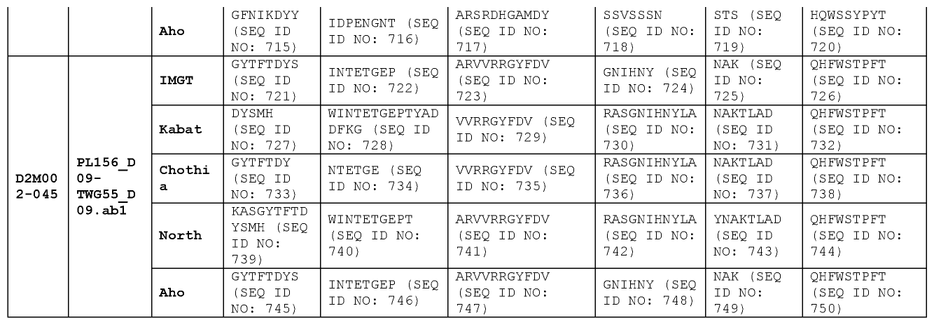

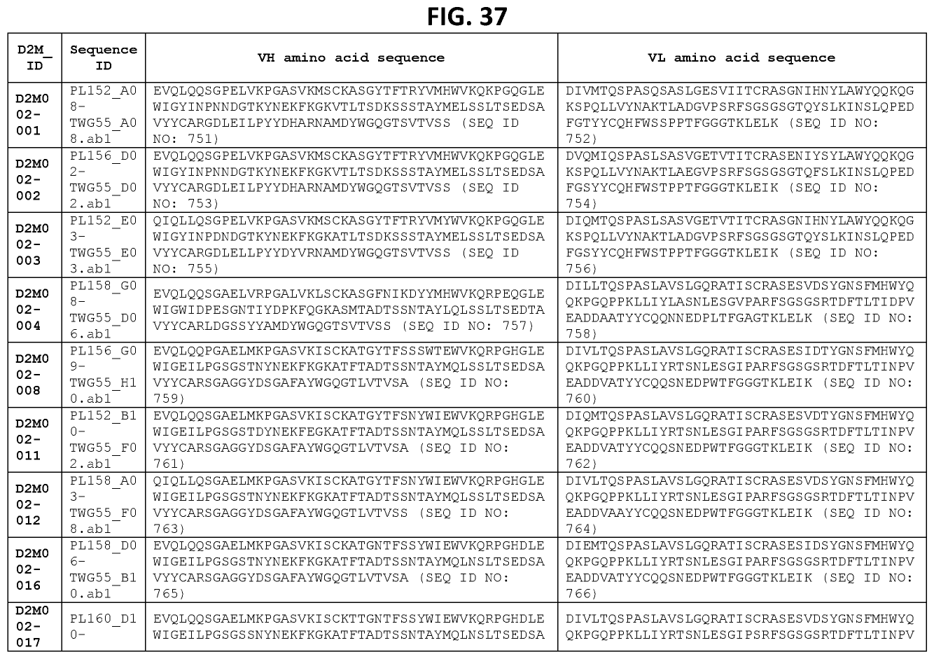

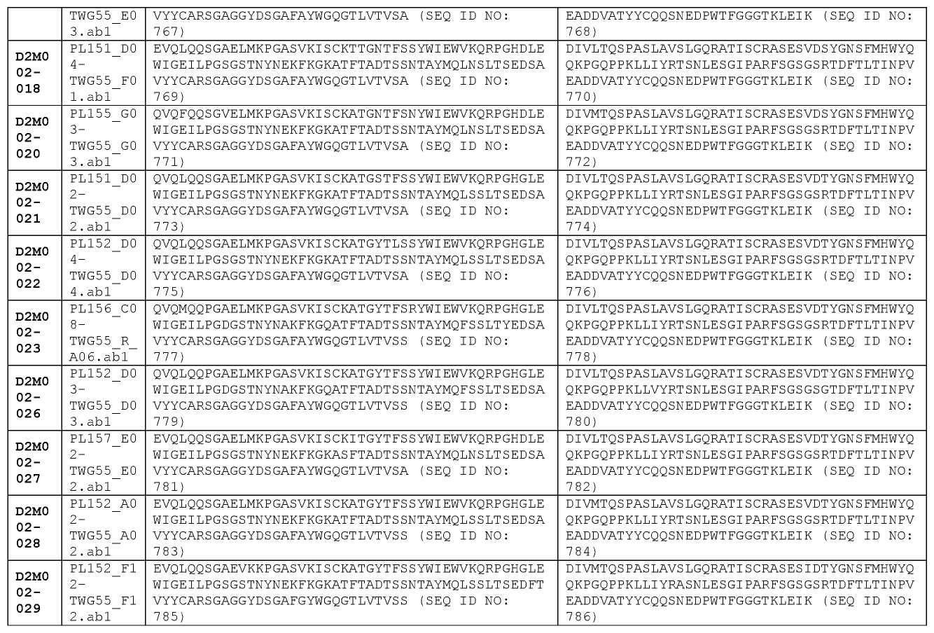

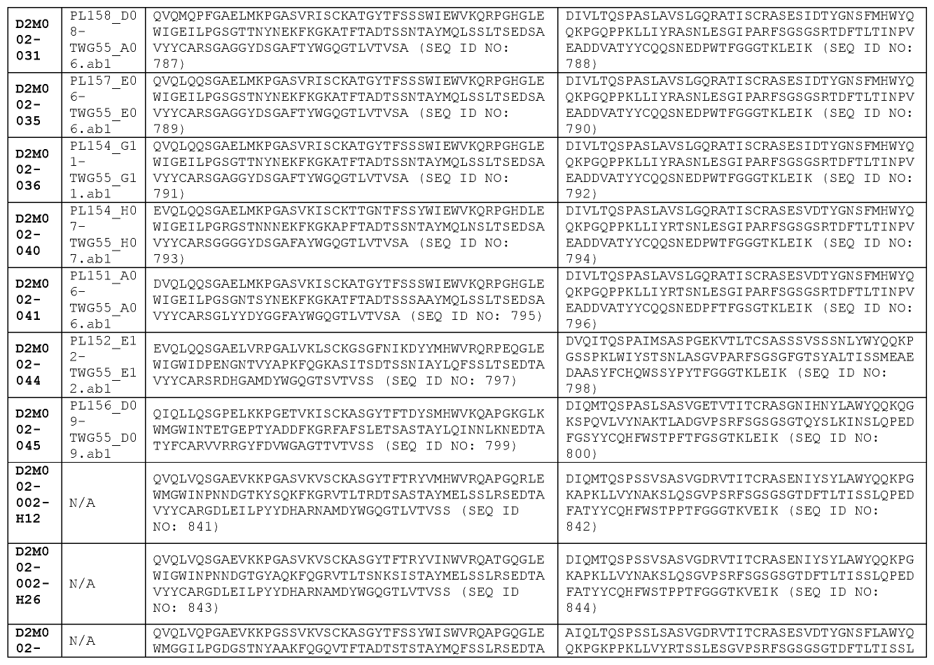

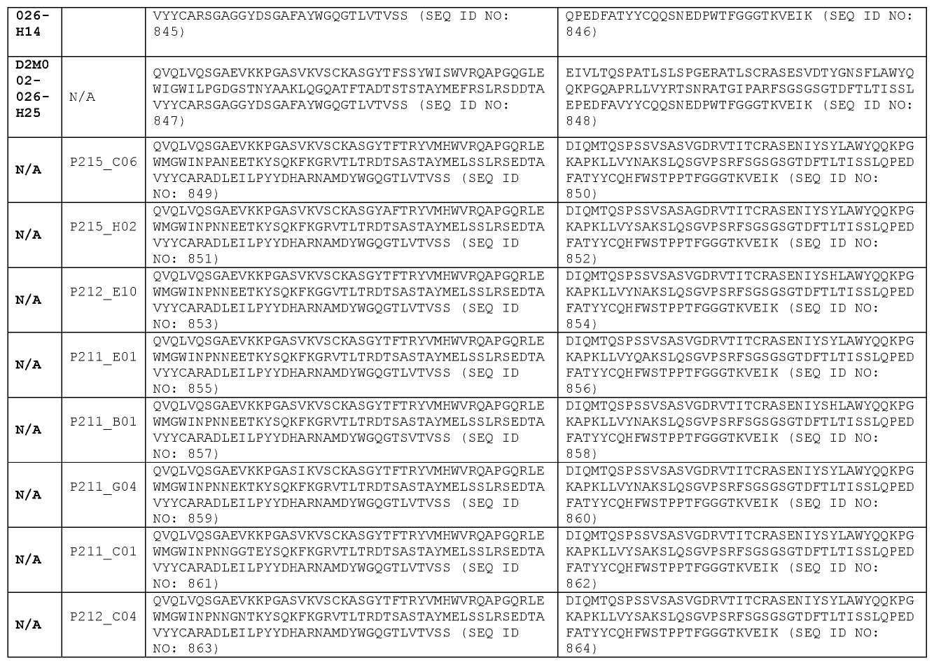

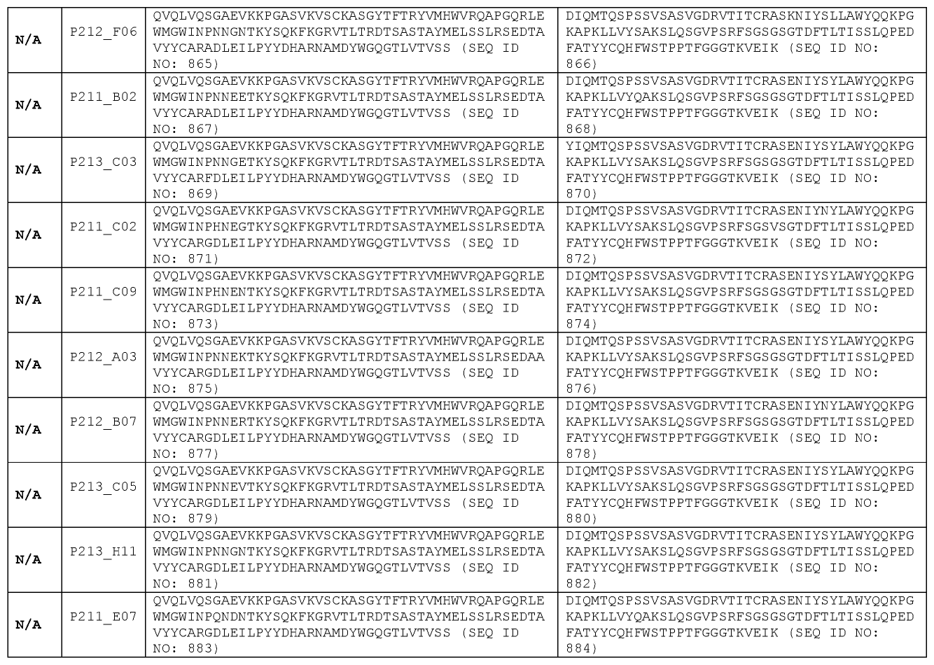

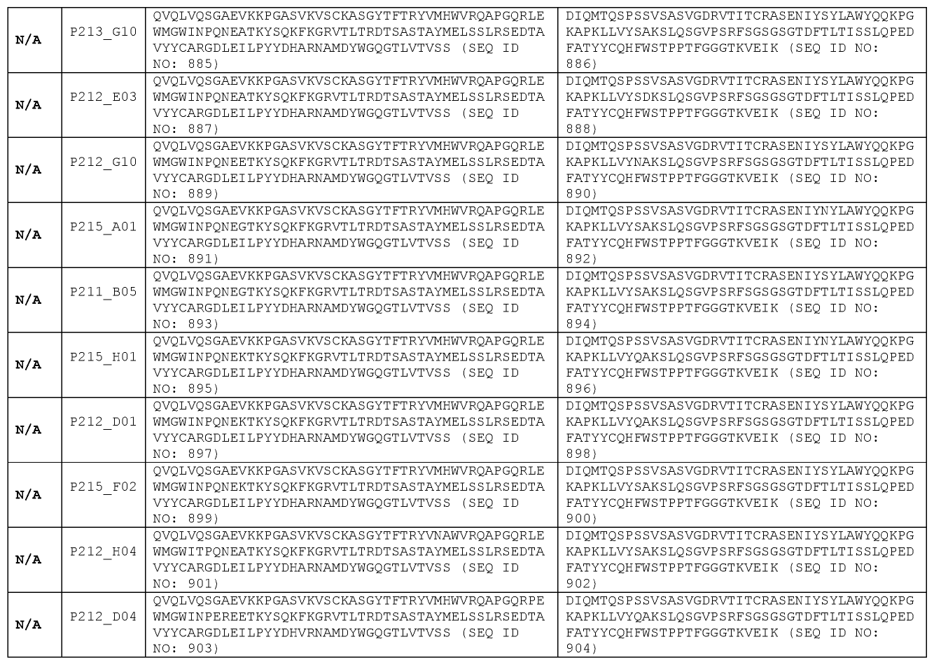

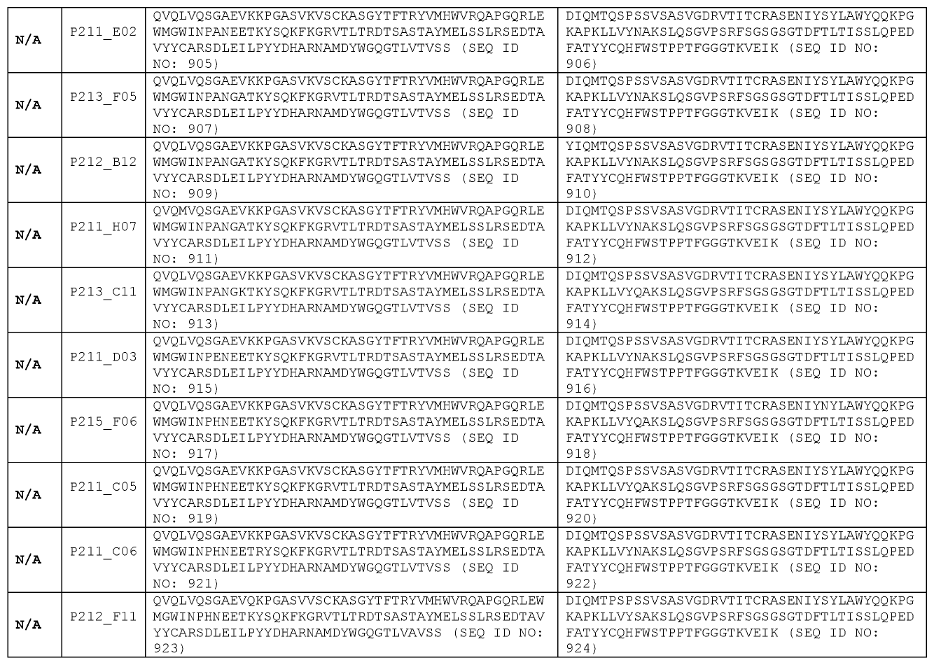

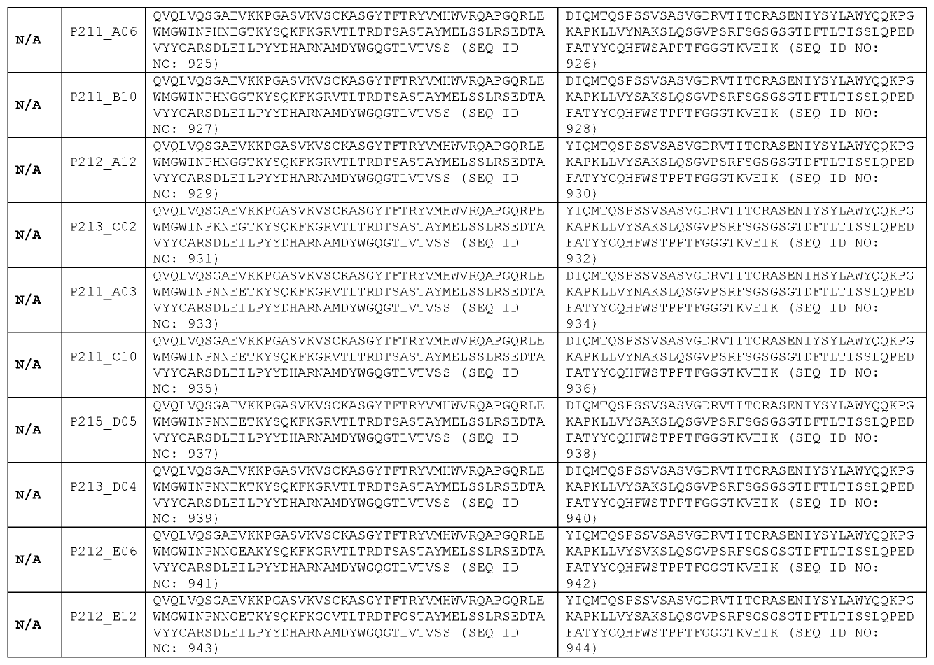

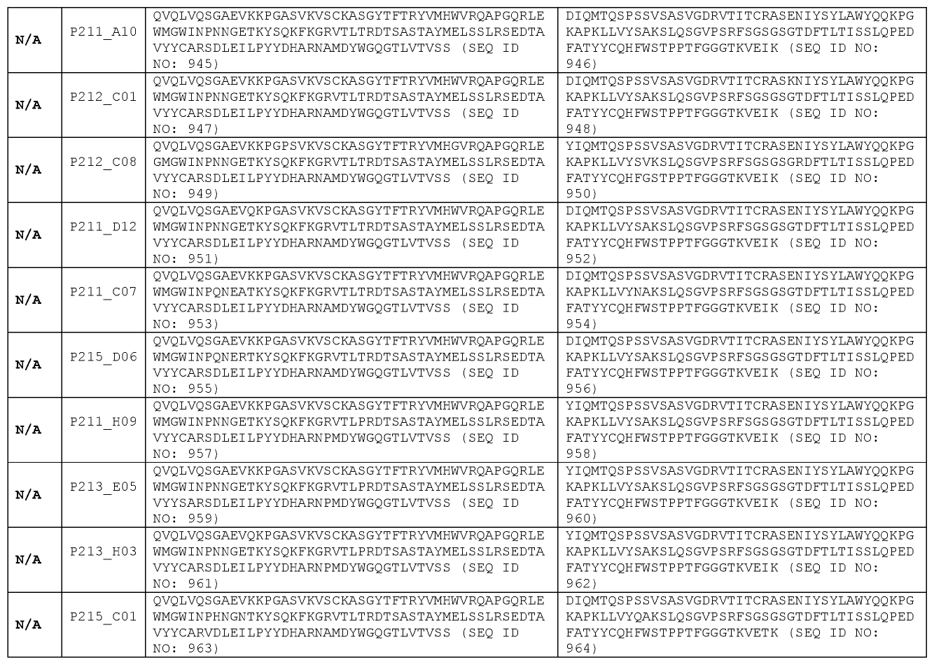

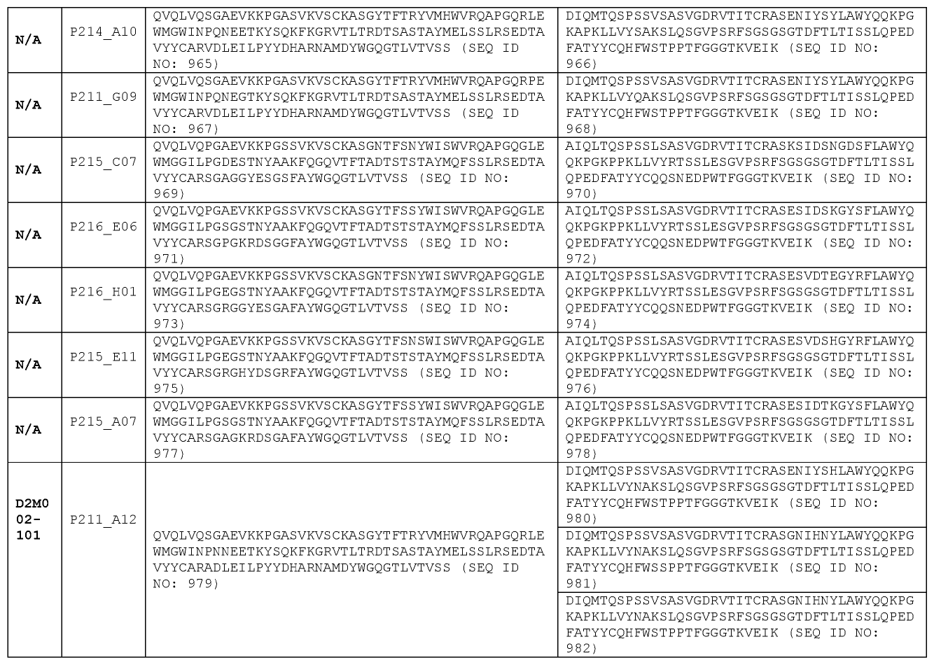

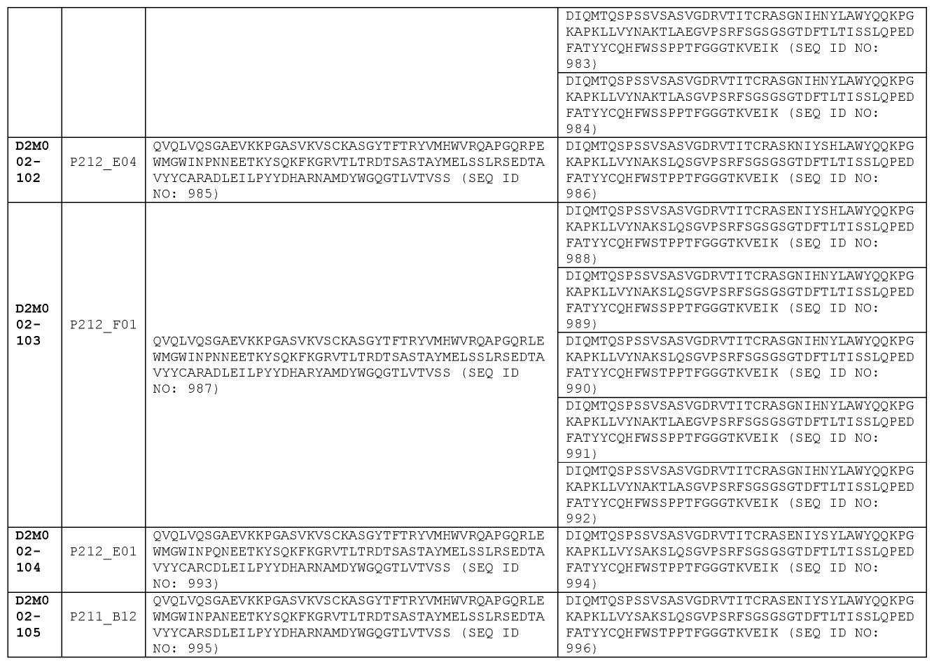

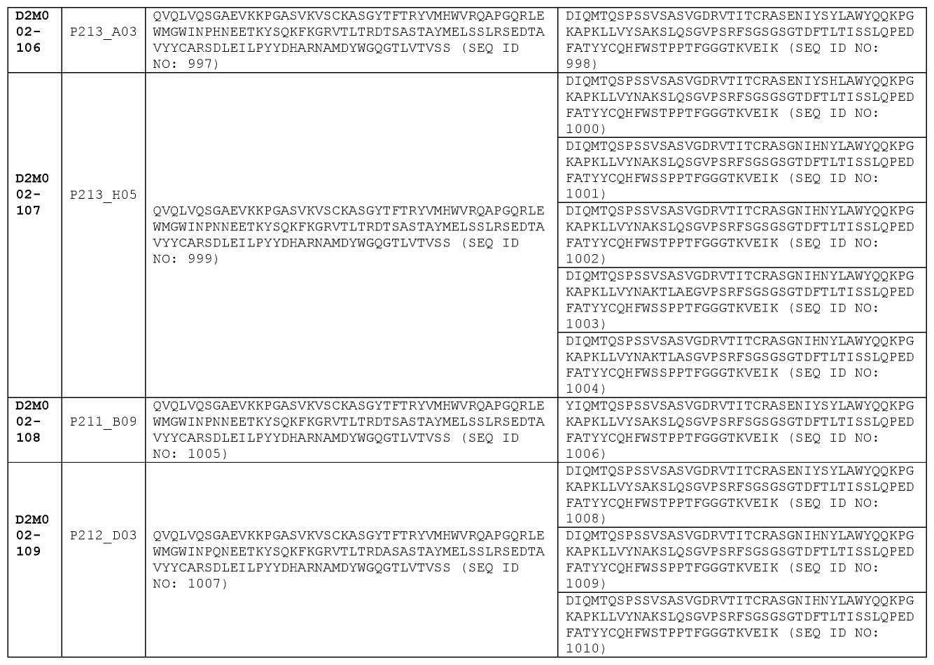

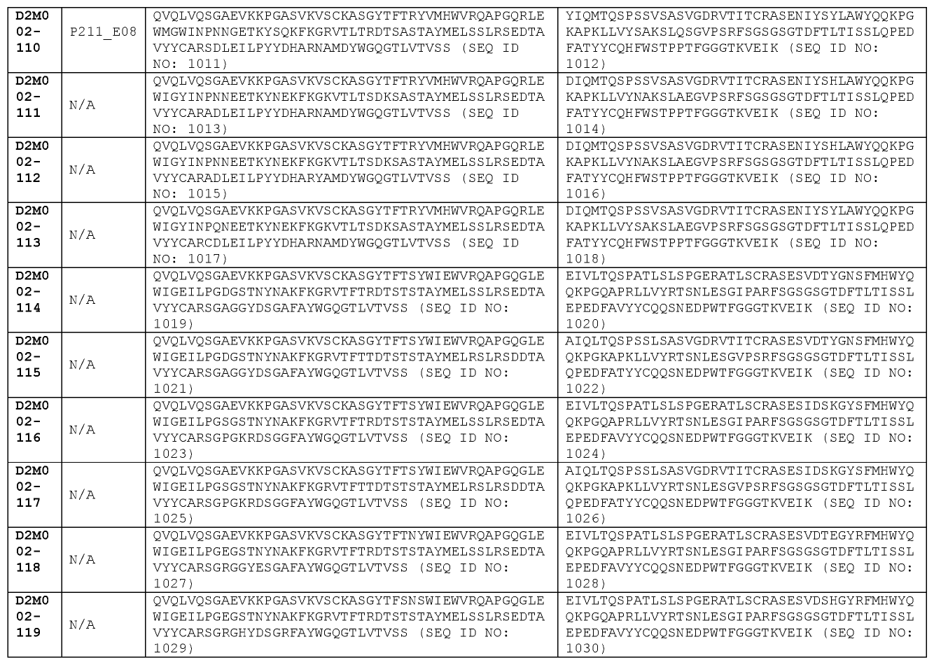

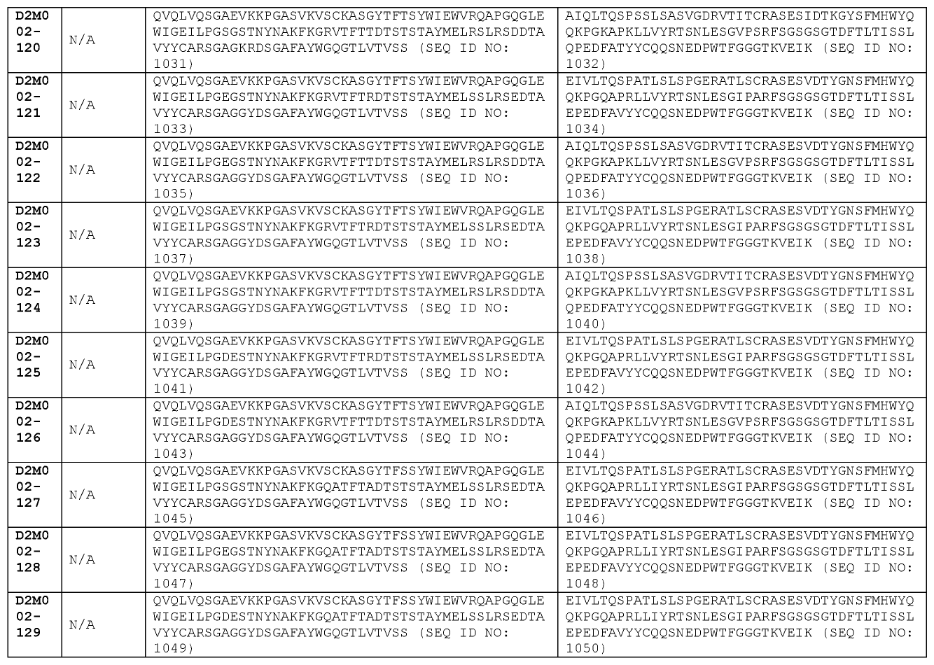

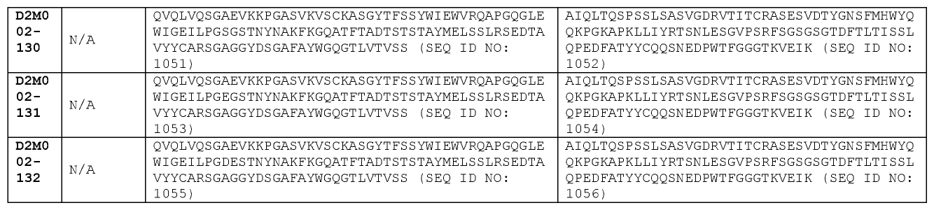

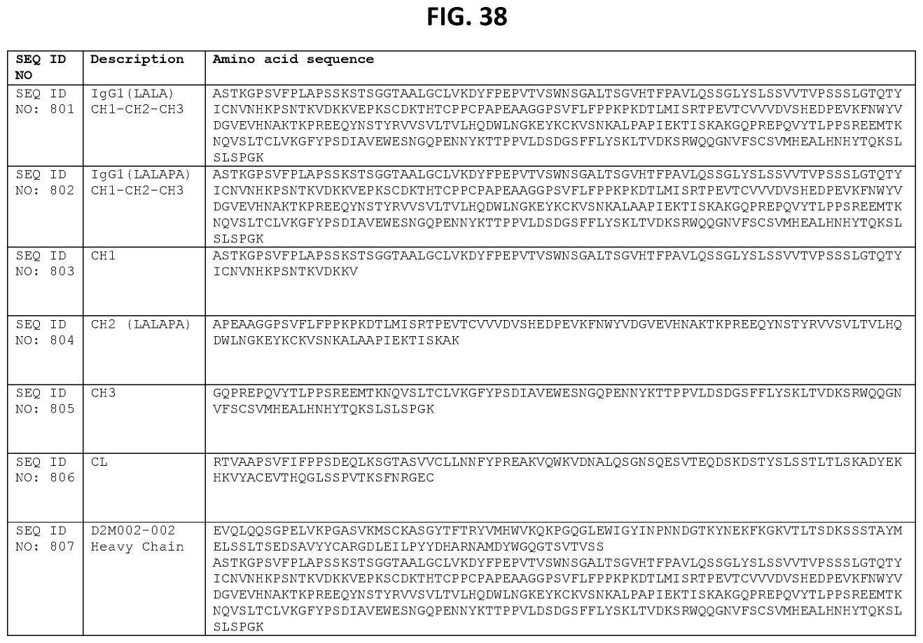

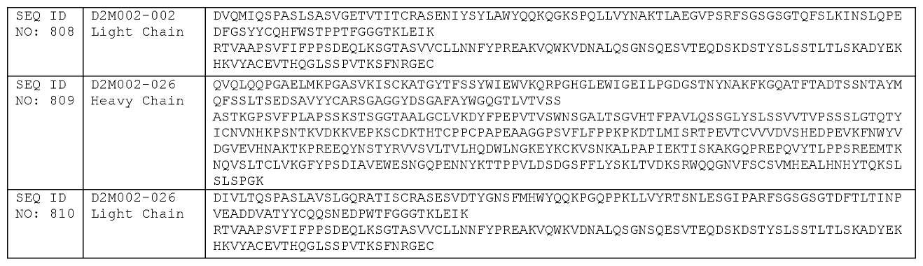

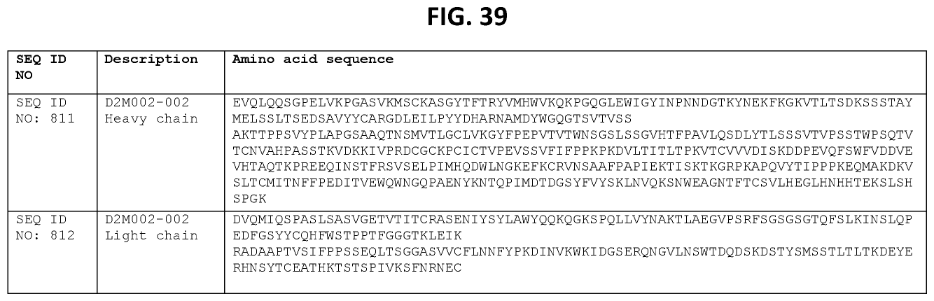

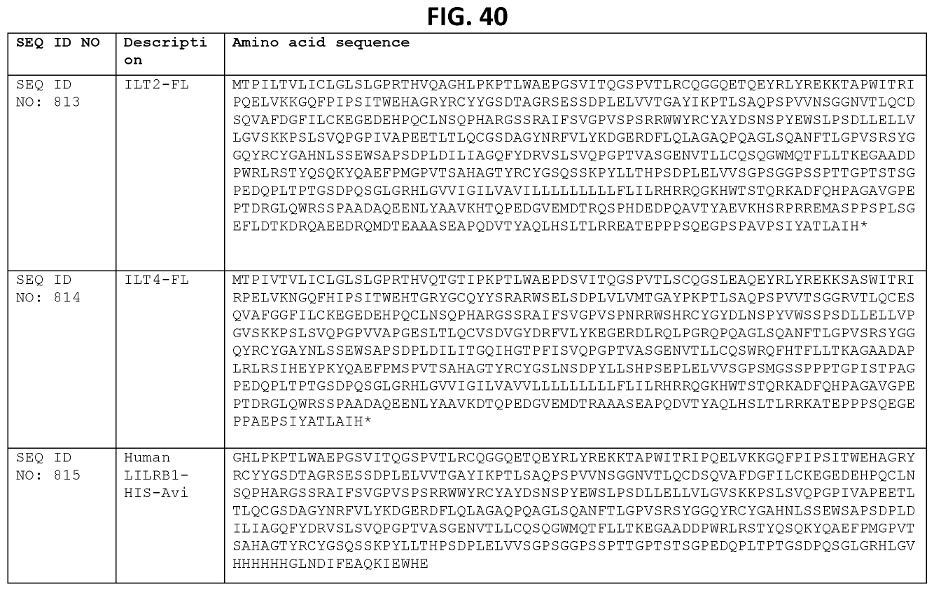

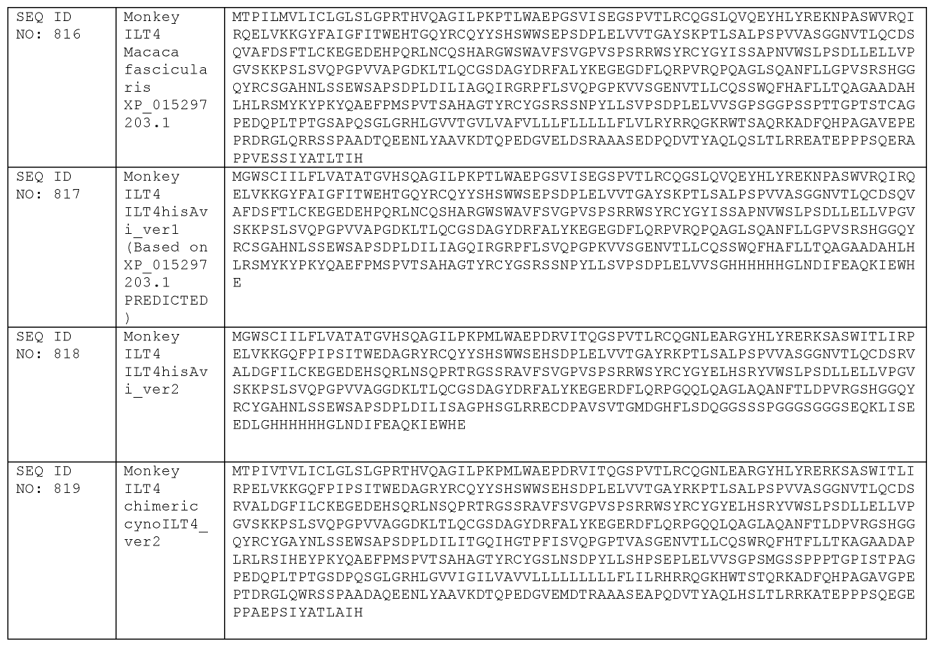

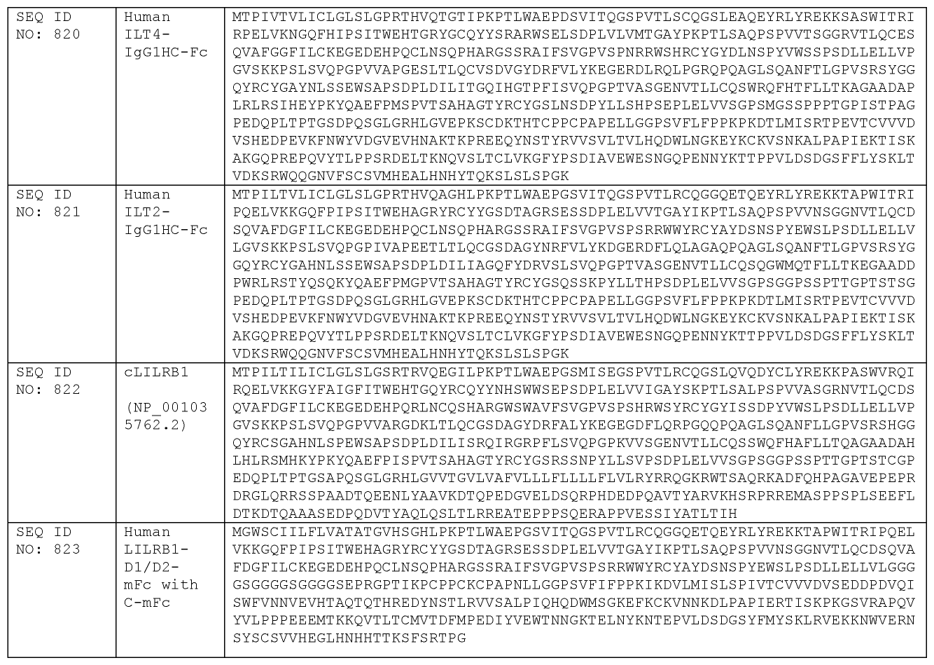

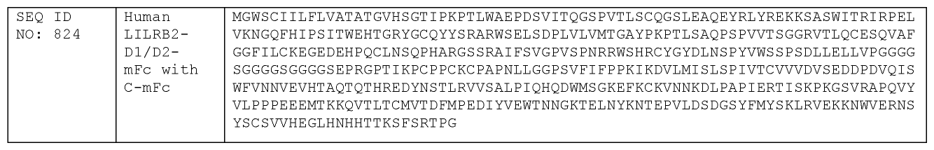

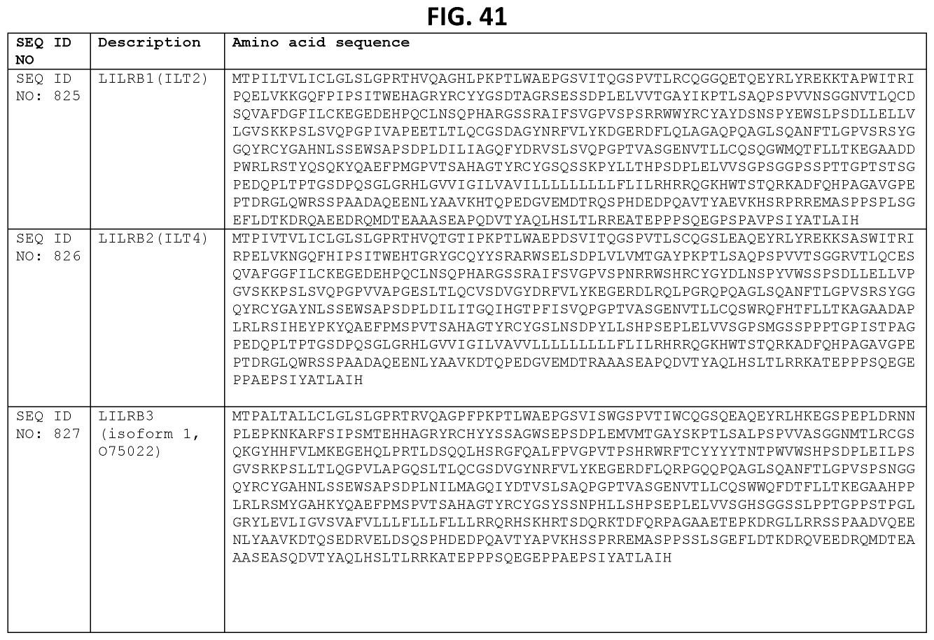

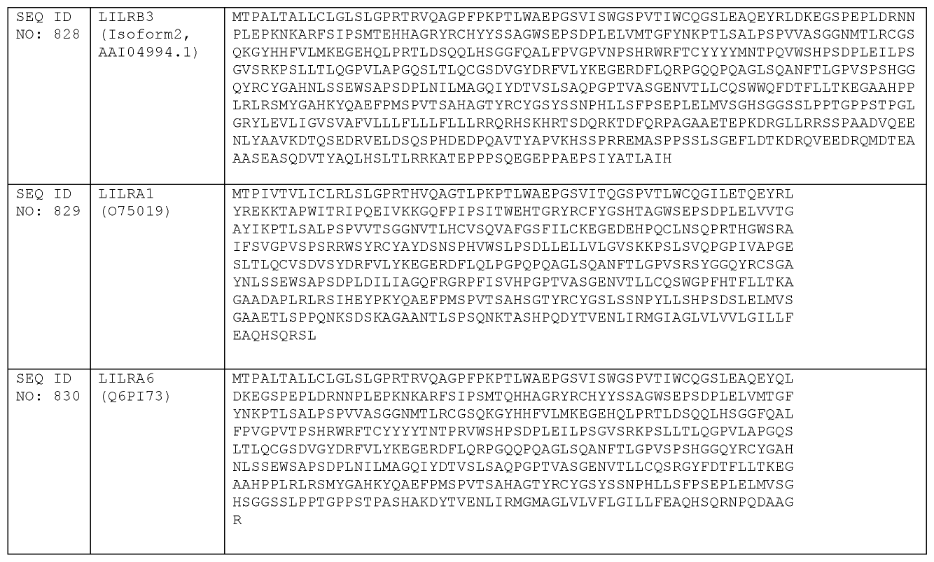

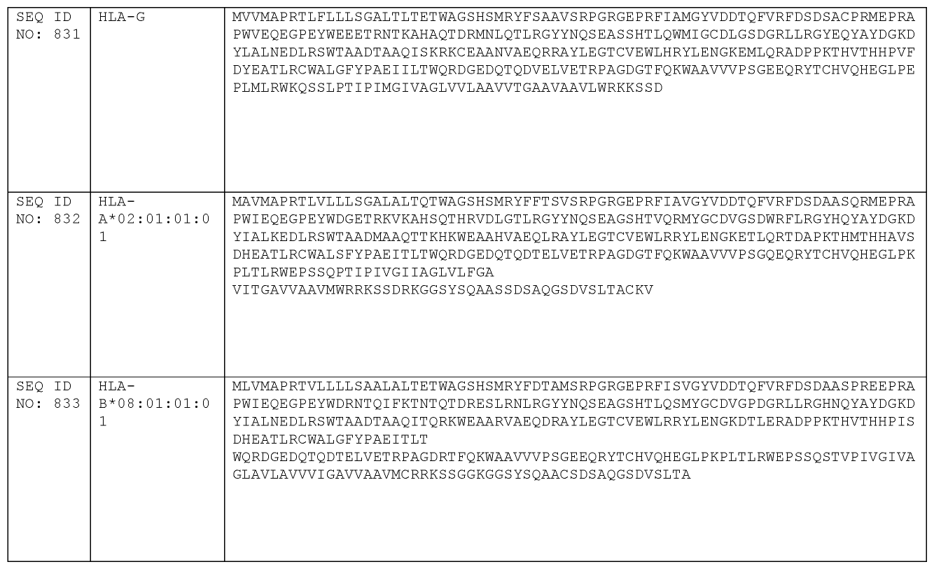

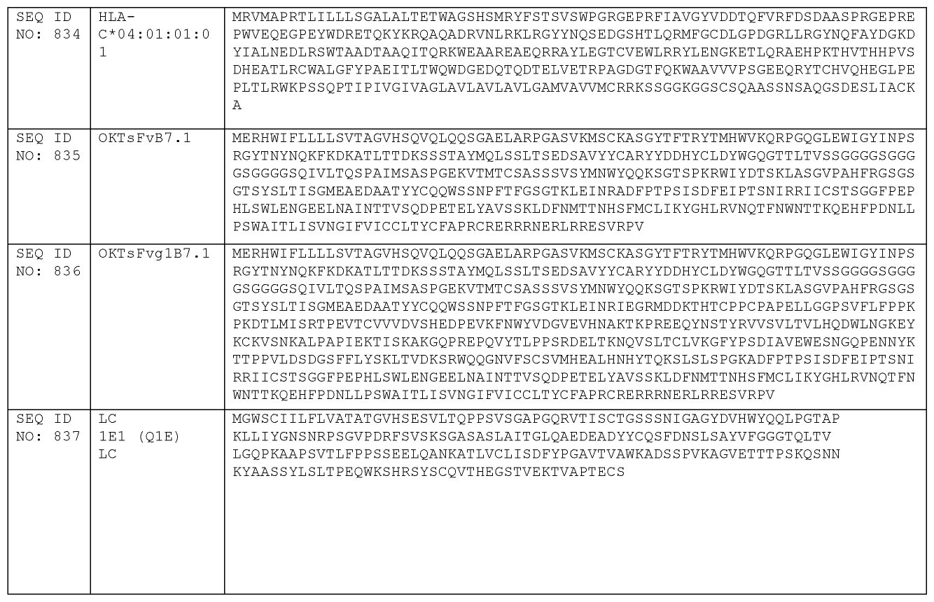

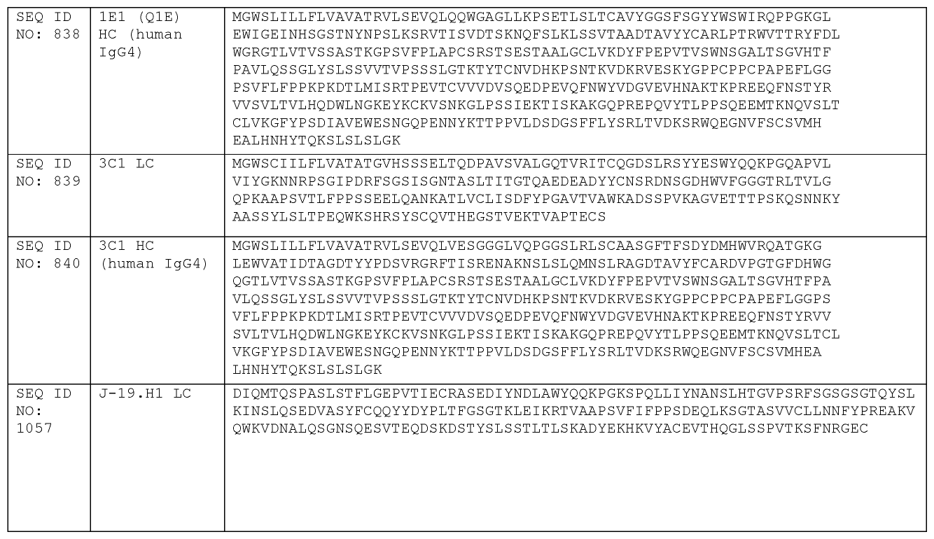

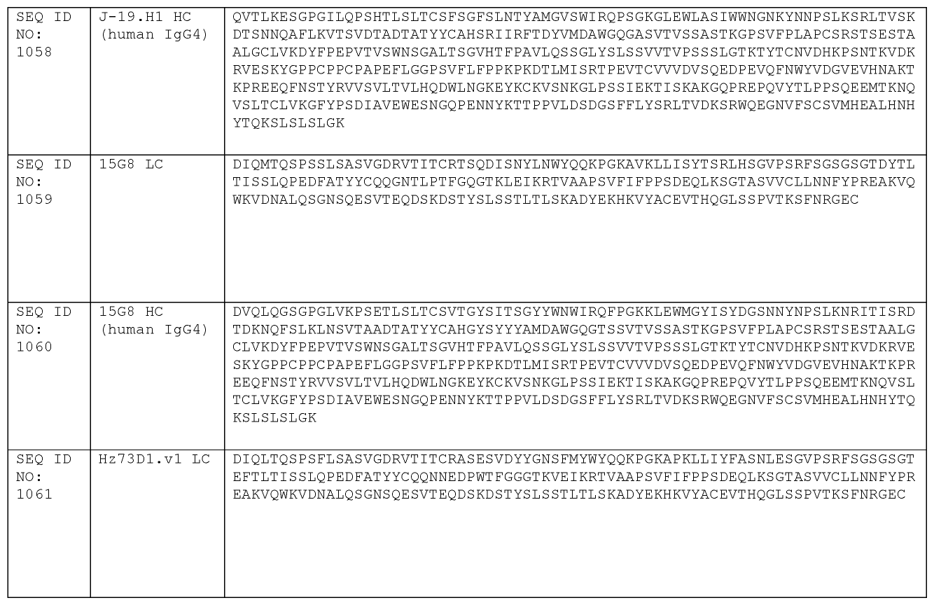

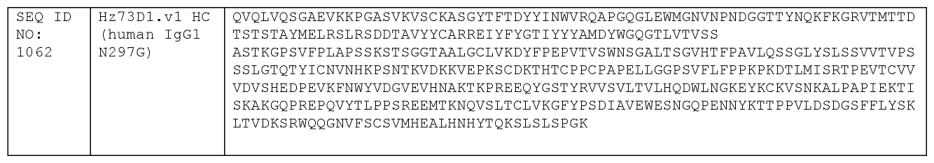

D2M002-126 exhibited a unique binning profile relative to D2M002-002. As shown in FIGS. 35A-35B, D2M002-126 and it parental D2M002-026 are distinct lineage from D2M002-103 and it parental D2M002-002. Loading D2M002-103 and it parental D2M002-002 first did not blocked the binding of D2M002-126 and it parental D2M002-026 to LILRB1 or LILRB2. However, loading D2M002-126 and it parental D2M002-026 first fully blocked the binding of D2M002-103 and it parental D2M002-002 to LILRB1 and partially blocked the binding of D2M002-103 and it parental D2M002-002 to LILRB2. The results indicate D2M002-103 and D2M002-126 have distinct binding epitopes, and D2M002-126 may affect D2M002-103 binding through steric hindrance.

D2M002-126 exhibited a unique binning profile relative to reference antibodies. As shown in FIGS. 35C-35D, D2M002-126 belongs to a bin different from the reference antibodies of 15G8, J-19.H1, 1E1(QE) and Hz73D1.v1. When 15G8 bound to LILRB1 first, 15G8 fully blocked the binding of D2M002-126 to LILRB1. However, when D2M002-126 bound to LILRB1 first, D2M002-126 did not block the binding of 15G8 to LILRB1. It suggests that D2M002-126 and 15G8 belong to different bins and have different binding epitopes on LILRB1. The competition in one direction may be due to steric hindrance. Although D2M002-126 binds to both LILRB1 and LILRB2, whereas 1E1(QE) only binds to LILRB2, D2M002-126 competes with 1E1(QE) for LILRB2 binding, suggesting that D2M002-126 and 1E1(QE) have overlapped epitope on LILRB2 binding while 1E1(QE) is lack of binding epitope on LILRB1. Although D2M002-126 and Hz73D1.v1 compete each other for binding to LILRB1 and LILRB2, D2M002-126 did not block the binding of J-19.H1 to LILRB2, whereas Hz73D1.v1 fully blocked the binding of J-19.H1 to LILRB2. Because Hz73D1 fell into J-19.H1 bin but D2M002-126 did not, D2M002-126 and Hz73D1.v1 belong to different bins. Therefore, D2M002-126 and Hz73D1.v1 have overlapped but distinct binding epitopes.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody or antigen-binding fragment thereof that binds to LILRB1 (Leukocyte immunoglobulin-like receptor subfamily B member 1) and/or LILRB2 (Leukocyte immunoglobulin-like receptor subfamily B member 2), comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 7, 8, 9, respectively, and the selected VL CDRs 1, 2, 3 amino acid sequences are set forth in SEQ ID NOs: 10, 11, 12, respectively;
(2) the selected VH CDRs 1, 2, 3 amino acid sequences are set forth in SEQ ID NOs: 37, 38, 39, respectively, and the selected VL CDRs 1, 2, 3 amino acid sequences are set forth in SEQ ID NOs: 40, 41, 42, respectively;
(3) the selected VH CDRs 1, 2, 3 amino acid sequences are set forth in SEQ ID NOs: 67, 68, 69, respectively, and the selected VL CDRs 1, 2, 3 amino acid sequences are set forth in SEQ ID NOs: 70, 71, 72, respectively;
(4) the selected VH CDRs 1, 2, 3 amino acid sequences are set forth in SEQ ID NOs: 97, 98, 99, respectively, and the selected VL CDRs 1, 2, 3 amino acid sequences are set forth in SEQ ID NOs: 100, 101, 102, respectively;
(5) the selected VH CDRs 1, 2, 3 amino acid sequences are set forth in SEQ ID NOs: 127, 128, 129, respectively, and the selected VL CDRs 1, 2, 3 amino acid sequences are set forth in SEQ ID NOs: 130, 131, 132, respectively;
(6) the selected VH CDRs 1, 2, 3 amino acid sequences are set forth in SEQ ID NOs: 157, 158, 159, respectively, and the selected VL CDRs 1, 2, 3 amino acid sequences are set forth in SEQ ID NOs: 160, 161, 162, respectively;
(7) the selected VH CDRs 1, 2, 3 amino acid sequences are set forth in SEQ ID NOs: 187, 188, 189, respectively, and the selected VL CDRs 1, 2, 3 amino acid sequences are set forth in SEQ ID NOs: 190, 191, 192, respectively;
(8) the selected VH CDRs 1, 2, 3 amino acid sequences are set forth in SEQ ID NOs: 217, 218, 219, respectively, and the selected VL CDRs 1, 2, 3 amino acid sequences are set forth in SEQ ID NOs: 220, 221, 222, respectively;
(9) the selected VH CDRs 1, 2, 3 amino acid sequences are set forth in SEQ ID NOs: 247, 248, 249, respectively, and the selected VL CDRs 1, 2, 3 amino acid sequences are set forth in SEQ ID NOs: 250, 251, 252, respectively;
(10) the selected VH CDRs 1, 2, 3 amino acid sequences are set forth in SEQ ID NOs: 277, 278, 279, respectively, and the selected VL CDRs 1, 2, 3 amino acid sequences are set forth in SEQ ID NOs: 280, 281, 282, respectively;
(11) the selected VH CDRs 1, 2, 3 amino acid sequences are set forth in SEQ ID NOs: 307, 308, 309, respectively, and the selected VL CDRs 1, 2, 3 amino acid sequences are set forth in SEQ ID NOs: 310, 311, 312, respectively;
(12) the selected VH CDRs 1, 2, 3 amino acid sequences are set forth in SEQ ID NOs: 337, 338, 339, respectively, and the selected VL CDRs 1, 2, 3 amino acid sequences are set forth in SEQ ID NOs: 340, 341, 342, respectively;
(13) the selected VH CDRs 1, 2, 3 amino acid sequences are set forth in SEQ ID NOs: 367, 368, 369, respectively, and the selected VL CDRs 1, 2, 3 amino acid sequences are set forth in SEQ ID NOs: 370, 371, 372, respectively;
(14) the selected VH CDRs 1, 2, 3 amino acid sequences are set forth in SEQ ID NOs: 397, 398, 399, respectively, and the selected VL CDRs 1, 2, 3 amino acid sequences are set forth in SEQ ID NOs: 400, 401, 402, respectively;
(15) the selected VH CDRs 1, 2, 3 amino acid sequences are set forth in SEQ ID NOs: 427, 428, 429, respectively, and the selected VL CDRs 1, 2, 3 amino acid sequences are set forth in SEQ ID NOs: 430, 431, 432, respectively;
(16) the selected VH CDRs 1, 2, 3 amino acid sequences are set forth in SEQ ID NOs: 457, 458, 459, respectively, and the selected VL CDRs 1, 2, 3 amino acid sequences are set forth in SEQ ID NOs: 460, 461, 462, respectively;
(17) the selected VH CDRs 1, 2, 3 amino acid sequences are set forth in SEQ ID NOs: 487, 488, 489, respectively, and the selected VL CDRs 1, 2, 3 amino acid sequences are set forth in SEQ ID NOs: 490, 491, 492, respectively;
(18) the selected VH CDRs 1, 2, 3 amino acid sequences are set forth in SEQ ID NOs: 517, 518, 519, respectively, and the selected VL CDRs 1, 2, 3 amino acid sequences are set forth in SEQ ID NOs: 520, 521, 522, respectively;
(19) the selected VH CDRs 1, 2, 3 amino acid sequences are set forth in SEQ ID NOs: 547, 548, 549, respectively, and the selected VL CDRs 1, 2, 3 amino acid sequences are set forth in SEQ ID NOs: 550, 551, 552, respectively;
(20) the selected VH CDRs 1, 2, 3 amino acid sequences are set forth in SEQ ID NOs: 577, 578, 579, respectively, and the selected VL CDRs 1, 2, 3 amino acid sequences are set forth in SEQ ID NOs: 580, 581, 582, respectively;
(21) the selected VH CDRs 1, 2, 3 amino acid sequences are set forth in SEQ ID NOs: 607, 608, 609, respectively, and the selected VL CDRs 1, 2, 3 amino acid sequences are set forth in SEQ ID NOs: 610, 611, 612, respectively;
(22) the selected VH CDRs 1, 2, 3 amino acid sequences are set forth in SEQ ID NOs: 637, 638, 639, respectively, and the selected VL CDRs 1, 2, 3 amino acid sequences are set forth in SEQ ID NOs: 640, 641, 642, respectively;
(23) the selected VH CDRs 1, 2, 3 amino acid sequences are set forth in SEQ ID NOs: 667, 668, 669, respectively, and the selected VL CDRs 1, 2, 3 amino acid sequences are set forth in SEQ ID NOs: 670, 671, 672, respectively;
(24) the selected VH CDRs 1, 2, 3 amino acid sequences are set forth in SEQ ID NOs: 697, 698, 699, respectively, and the selected VL CDRs 1, 2, 3 amino acid sequences are set forth in SEQ ID NOs: 700, 701, 702, respectively;
(25) the selected VH CDRs 1, 2, 3 amino acid sequences are set forth in SEQ ID NOs: 727, 728, 729, respectively, and the selected VL CDRs 1, 2, 3 amino acid sequences are set forth in SEQ ID NOs: 730, 731, 732, respectively;
(26) the selected VH CDRs 1, 2, 3 amino acid sequences are set forth in SEQ ID NOs: 1069, 1070, 1071, respectively, and the selected VL CDRs 1, 2, 3 amino acid sequences are set forth in SEQ ID NOs: 1072, 1073, 1074, respectively;
(27) the selected VH CDRs 1, 2, 3 amino acid sequences are set forth in SEQ ID NOs: 1093, 1094, 1095, respectively, and the selected VL CDRs 1, 2, 3 amino acid sequences are set forth in SEQ ID NOs: 1096, 1097, 1098, respectively;
(28) the selected VH CDRs 1, 2, 3 amino acid sequences are set forth in SEQ ID NOs: 1117, 1118, 1119, respectively, and the selected VL CDRs 1, 2, 3 amino acid sequences are set forth in SEQ ID NOs: 1120, 1121, 1122, respectively;
(29) the selected VH CDRs 1, 2, 3 amino acid sequences are set forth in SEQ ID NOs: 1141, 1142, 1143, respectively, and the selected VL CDRs 1, 2, 3 amino acid sequences are set forth in SEQ ID NOs: 1144, 1145, 1146, respectively;
(30) the selected VH CDRs 1, 2, 3 amino acid sequences are set forth in SEQ ID NOs: 1165, 1166, 1167, respectively, and the selected VL CDRs 1, 2, 3 amino acid sequences are set forth in SEQ ID NOs: 1168, 1169, 1170, respectively;
(31) the selected VH CDRs 1, 2, 3 amino acid sequences are set forth in SEQ ID NOs: 1189, 1190, 1191, respectively, and the selected VL CDRs 1, 2, 3 amino acid sequences are set forth in SEQ ID NOs: 1192, 1193, 1194, respectively;
(32) the selected VH CDRs 1, 2, 3 amino acid sequences are set forth in SEQ ID NOs: 1213, 1214, 1215, respectively, and the selected VL CDRs 1, 2, 3 amino acid sequences are set forth in SEQ ID NOs: 1216, 1217, 1218, respectively;
(33) the selected VH CDRs 1, 2, 3 amino acid sequences are set forth in SEQ ID NOs: 1237, 1238, 1239, respectively, and the selected VL CDRs 1, 2, 3 amino acid sequences are set forth in SEQ ID NOs: 1240, 1241, 1242, respectively;
(34) the selected VH CDRs 1, 2, 3 amino acid sequences are set forth in SEQ ID NOs: 1261, 1262, 1263, respectively, and the selected VL CDRs 1, 2, 3 amino acid sequences are set forth in SEQ ID NOs: 1264, 1265, 1266, respectively;
(35) the selected VH CDRs 1, 2, 3 amino acid sequences are set forth in SEQ ID NOs: 1285, 1286, 1287, respectively, and the selected VL CDRs 1, 2, 3 amino acid sequences are set forth in SEQ ID NOs: 1288, 1289, 1290, respectively;
(36) the selected VH CDRs 1, 2, 3 amino acid sequences are set forth in SEQ ID NOs: 1309, 1310, 1311, respectively, and the selected VL CDRs 1, 2, 3 amino acid sequences are set forth in SEQ ID NOs: 1312, 1313, 1314, respectively;
(37) the selected VH CDRs 1, 2, 3 amino acid sequences are set forth in SEQ ID NOs: 1333, 1334, 1335, respectively, and the selected VL CDRs 1, 2, 3 amino acid sequences are set forth in SEQ ID NOs: 1336, 1337, 1338, respectively;
(38) the selected VH CDRs 1, 2, 3 amino acid sequences are set forth in SEQ ID NOs: 1357, 1358, 1359, respectively, and the selected VL CDRs 1, 2, 3 amino acid sequences are set forth in SEQ ID NOs: 1360, 1361, 1362, respectively;
(39) the selected VH CDRs 1, 2, 3 amino acid sequences are set forth in SEQ ID NOs: 1381, 1382, 1383, respectively, and the selected VL CDRs 1, 2, 3 amino acid sequences are set forth in SEQ ID NOs: 1384, 1385, 1386, respectively;
(40) the selected VH CDRs 1, 2, 3 amino acid sequences are set forth in SEQ ID NOs: 1405, 1406, 1407, respectively, and the selected VL CDRs 1, 2, 3 amino acid sequences are set forth in SEQ ID NOs: 1408, 1409, 1410, respectively;
(41) the selected VH CDRs 1, 2, 3 amino acid sequences are set forth in SEQ ID NOs: 1429, 1430, 1431, respectively, and the selected VL CDRs 1, 2, 3 amino acid sequences are set forth in SEQ ID NOs: 1432, 1433, 1434, respectively;
(42) the selected VH CDRs 1, 2, 3 amino acid sequences are set forth in SEQ ID NOs: 1453, 1454, 1455, respectively, and the selected VL CDRs 1, 2, 3 amino acid sequences are set forth in SEQ ID NOs: 1456, 1457, 1458, respectively;
(43) the selected VH CDRs 1, 2, 3 amino acid sequences are set forth in SEQ ID NOs: 1477, 1478, 1479, respectively, and the selected VL CDRs 1, 2, 3 amino acid sequences are set forth in SEQ ID NOs: 1480, 1481, 1482, respectively;
(44) the selected VH CDRs 1, 2, 3 amino acid sequences are set forth in SEQ ID NOs: 1501, 1502, 1503, respectively, and the selected VL CDRs 1, 2, 3 amino acid sequences are set forth in SEQ ID NOs: 1504, 1505, 1506, respectively;
(45) the selected VH CDRs 1, 2, 3 amino acid sequences are set forth in SEQ ID NOs: 1525, 1526, 1527, respectively, and the selected VL CDRs 1, 2, 3 amino acid sequences are set forth in SEQ ID NOs: 1528, 1529, 1530, respectively;
(46) the selected VH CDRs 1, 2, 3 amino acid sequences are set forth in SEQ ID NOs: 1549, 1550, 1551, respectively, and the selected VL CDRs 1, 2, 3 amino acid sequences are set forth in SEQ ID NOs: 1552, 1553, 1554, respectively;
(47) the selected VH CDRs 1, 2, 3 amino acid sequences are set forth in SEQ ID NOs: 1573, 1574, 1575, respectively, and the selected VL CDRs 1, 2, 3 amino acid sequences are set forth in SEQ ID NOs: 1576, 1577, 1578, respectively;
(48) the selected VH CDRs 1, 2, 3 amino acid sequences are set forth in SEQ ID NOs: 1597, 1598, 1599, respectively, and the selected VL CDRs 1, 2, 3 amino acid sequences are set forth in SEQ ID NOs: 1600, 1601, 1602, respectively;
(49) the selected VH CDRs 1, 2, 3 amino acid sequences are set forth in SEQ ID NOs: 1621, 1622, 1623, respectively, and the selected VL CDRs 1, 2, 3 amino acid sequences are set forth in SEQ ID NOs: 1624, 1625, 1626, respectively;
(50) the selected VH CDRs 1, 2, 3 amino acid sequences are set forth in SEQ ID NOs: 1645, 1646, 1647, respectively, and the selected VL CDRs 1, 2, 3 amino acid sequences are set forth in SEQ ID NOs: 1648, 1649, 1650, respectively; and
(51) the selected VH CDRs 1, 2, 3 amino acid sequences are set forth in SEQ ID NOs: 1669, 1670, 1671, respectively, and the selected VL CDRs 1, 2, 3 amino acid sequences are set forth in SEQ ID NOs: 1672, 1673, 1674, respectively.

2. The antibody or antigen-binding fragment thereof of claim 1, wherein the VH comprises CDRs 1, 2, 3 with the amino acid sequences set forth in SEQ ID NOs: 7, 8, and 9 respectively, and the VL comprises CDRs 1, 2, 3 with the amino acid sequences set forth in SEQ ID NOs: 10, 11, and 12, respectively.

3. The antibody or antigen-binding fragment thereof of claim 1, wherein the VH comprises CDRs 1, 2, 3 with the amino acid sequences set forth in SEQ ID NOs: 37, 38, and 39 respectively, and the VL comprises CDRs 1, 2, 3 with the amino acid sequences set forth in SEQ ID NOs: 40, 41, and 42, respectively.

4. The antibody or antigen-binding fragment thereof of claim 1, wherein the VH comprises CDRs 1, 2, 3 with the amino acid sequences set forth in SEQ ID NOs: 67, 68, and 69 respectively, and the VL comprises CDRs 1, 2, 3 with the amino acid sequences set forth in SEQ ID NOs: 70, 71, and 72, respectively.

5. The antibody or antigen-binding fragment thereof of claim 1, wherein the VH comprises CDRs 1, 2, 3 with the amino acid sequences set forth in SEQ ID NOs: 97, 98, and 99 respectively, and the VL comprises CDRs 1, 2, 3 with the amino acid sequences set forth in SEQ ID NOs: 100, 101, and 102, respectively.

6. The antibody or antigen-binding fragment thereof of claim 1, wherein the VH comprises CDRs 1, 2, 3 with the amino acid sequences set forth in SEQ ID NOs: 127, 128, and 129 respectively, and the VL comprises CDRs 1, 2, 3 with the amino acid sequences set forth in SEQ ID NOs: 130, 131, and 132, respectively.

7. The antibody or antigen-binding fragment thereof of claim 1, wherein the VH comprises CDRs 1, 2, 3 with the amino acid sequences set forth in SEQ ID NOs: 157, 158, and 159 respectively, and the VL comprises CDRs 1, 2, 3 with the amino acid sequences set forth in SEQ ID NOs: 160, 161, and 162, respectively.

8. The antibody or antigen-binding fragment thereof of claim 1, wherein the VH comprises CDRs 1, 2, 3 with the amino acid sequences set forth in SEQ ID NOs: 187, 188, and 189 respectively, and the VL comprises CDRs 1, 2, 3 with the amino acid sequences set forth in SEQ ID NOs: 190, 191, and 192, respectively.

9. The antibody or antigen-binding fragment thereof of claim 1, wherein the VH comprises CDRs 1, 2, 3 with the amino acid sequences set forth in SEQ ID NOs: 217, 218, and 219 respectively, and the VL comprises CDRs 1, 2, 3 with the amino acid sequences set forth in SEQ ID NOs: 220, 221, and 222, respectively.

10. The antibody or antigen-binding fragment thereof of claim 1, wherein the VH comprises CDRs 1, 2, 3 with the amino acid sequences set forth in SEQ ID NOs: 247, 248, and 249 respectively, and the VL comprises CDRs 1, 2, 3 with the amino acid sequences set forth in SEQ ID NOs: 250, 251, and 252, respectively.

11. The antibody or antigen-binding fragment thereof of claim 1, wherein the VH comprises CDRs 1, 2, 3 with the amino acid sequences set forth in SEQ ID NOs: 277, 278, and 279 respectively, and the VL comprises CDRs 1, 2, 3 with the amino acid sequences set forth in SEQ ID NOs: 280, 281, and 282, respectively.

12. The antibody or antigen-binding fragment thereof of claim 1, wherein the VH comprises CDRs 1, 2, 3 with the amino acid sequences set forth in SEQ ID NOs: 307, 308, and 309 respectively, and the VL comprises CDRs 1, 2, 3 with the amino acid sequences set forth in SEQ ID NOs: 310, 311, and 312, respectively.

13. The antibody or antigen-binding fragment thereof of claim 1, wherein the VH comprises CDRs 1, 2, 3 with the amino acid sequences set forth in SEQ ID NOs: 337, 338, and 339 respectively, and the VL comprises CDRs 1, 2, 3 with the amino acid sequences set forth in SEQ ID NOs: 340, 341, and 342, respectively.

14. The antibody or antigen-binding fragment thereof of claim 1, wherein the VH comprises CDRs 1, 2, 3 with the amino acid sequences set forth in SEQ ID NOs: 367, 368, and 369 respectively, and the VL comprises CDRs 1, 2, 3 with the amino acid sequences set forth in SEQ ID NOs: 370, 371, and 372, respectively.

15. The antibody or antigen-binding fragment thereof of claim 1, wherein the VH comprises CDRs 1, 2, 3 with the amino acid sequences set forth in SEQ ID NOs: 397, 398, and 399 respectively, and the VL comprises CDRs 1, 2, 3 with the amino acid sequences set forth in SEQ ID NOs: 400, 401, and 402, respectively.

16. The antibody or antigen-binding fragment thereof of claim 1, wherein the VH comprises CDRs 1, 2, 3 with the amino acid sequences set forth in SEQ ID NOs: 427, 428, and 429 respectively, and the VL comprises CDRs 1, 2, 3 with the amino acid sequences set forth in SEQ ID NOs: 430, 431, and 432, respectively.

17. The antibody or antigen-binding fragment thereof of claim 1, wherein the VH comprises CDRs 1, 2, 3 with the amino acid sequences set forth in SEQ ID NOs: 457, 458, and 459 respectively, and the VL comprises CDRs 1, 2, 3 with the amino acid sequences set forth in SEQ ID NOs: 460, 461, and 462, respectively.

18. The antibody or antigen-binding fragment thereof of claim 1, wherein the VH comprises CDRs 1, 2, 3 with the amino acid sequences set forth in SEQ ID NOs: 487, 488, and 489 respectively, and the VL comprises CDRs 1, 2, 3 with the amino acid sequences set forth in SEQ ID NOs: 490, 491, and 492, respectively.

19. The antibody or antigen-binding fragment thereof of claim 1, wherein the VH comprises CDRs 1, 2, 3 with the amino acid sequences set forth in SEQ ID NOs: 517, 518, and 519 respectively, and the VL comprises CDRs 1, 2, 3 with the amino acid sequences set forth in SEQ ID NOs: 520, 521, and 522, respectively.

20. The antibody or antigen-binding fragment thereof of claim 1, wherein the VH comprises CDRs 1, 2, 3 with the amino acid sequences set forth in SEQ ID NOs: 547, 548, and 549 respectively, and the VL comprises CDRs 1, 2, 3 with the amino acid sequences set forth in SEQ ID NOs: 550, 551, and 552, respectively.

21. The antibody or antigen-binding fragment thereof of claim 1, wherein the VH comprises CDRs 1, 2, 3 with the amino acid sequences set forth in SEQ ID NOs: 577, 578, and 579 respectively, and the VL comprises CDRs 1, 2, 3 with the amino acid sequences set forth in SEQ ID NOs: 580, 581, and 582, respectively.

22. The antibody or antigen-binding fragment thereof of claim 1, wherein the VH comprises CDRs 1, 2, 3 with the amino acid sequences set forth in SEQ ID NOs: 607, 608, and 609 respectively, and the VL comprises CDRs 1, 2, 3 with the amino acid sequences set forth in SEQ ID NOs: 610, 611, and 612, respectively.

23. The antibody or antigen-binding fragment thereof of claim 1, wherein the VH comprises CDRs 1, 2, 3 with the amino acid sequences set forth in SEQ ID NOs: 637, 638, and 639 respectively, and the VL comprises CDRs 1, 2, 3 with the amino acid sequences set forth in SEQ ID NOs: 640, 641, and 642, respectively.

24. The antibody or antigen-binding fragment thereof of claim 1, wherein the VH comprises CDRs 1, 2, 3 with the amino acid sequences set forth in SEQ ID NOs: 667, 668, and 669 respectively, and the VL comprises CDRs 1, 2, 3 with the amino acid sequences set forth in SEQ ID NOs: 670, 671, and 672, respectively.

25. The antibody or antigen-binding fragment thereof of claim 1, wherein the VH comprises CDRs 1, 2, 3 with the amino acid sequences set forth in SEQ ID NOs: 697, 698, and 699 respectively, and the VL comprises CDRs 1, 2, 3 with the amino acid sequences set forth in SEQ ID NOs: 700, 701, and 702, respectively.

26. The antibody or antigen-binding fragment thereof of claim 1, wherein the VH comprises CDRs 1, 2, 3 with the amino acid sequences set forth in SEQ ID NOs: 727, 728, and 729 respectively, and the VL comprises CDRs 1, 2, 3 with the amino acid sequences set forth in SEQ ID NOs: 730, 731, and 732, respectively.

27. The antibody or antigen-binding fragment thereof of claim 1, wherein the VH comprises CDRs 1, 2, 3 with the amino acid sequences set forth in SEQ ID NOs: 1069, 1070, and 1071, respectively, and the VL comprises CDRs 1, 2, 3 with the amino acid sequences set forth in SEQ ID NOs: 1072, 1073, and 1074, respectively.

28. The antibody or antigen-binding fragment thereof of claim 1, wherein the VH comprises CDRs 1, 2, 3 with the amino acid sequences set forth in SEQ ID NOs: 1093, 1094, and 1095, respectively, and the VL comprises CDRs 1, 2, 3 with the amino acid sequences set forth in SEQ ID NOs: 1096, 1097, and 1098, respectively.

29. The antibody or antigen-binding fragment thereof of claim 1, wherein the VH comprises CDRs 1, 2, 3 with the amino acid sequences set forth in SEQ ID NOs: 1117, 1118, and 1119, respectively, and the VL comprises CDRs 1, 2, 3 with the amino acid sequences set forth in SEQ ID NOs: 1120, 1121, and 1122, respectively.

30. The antibody or antigen-binding fragment thereof of claim 1, wherein the VH comprises CDRs 1, 2, 3 with the amino acid sequences set forth in SEQ ID NOs: 1141, 1142, and 1143, respectively, and the VL comprises CDRs 1, 2, 3 with the amino acid sequences set forth in SEQ ID NOs: 1144, 1145, and 1146, respectively.

31. The antibody or antigen-binding fragment thereof of claim 1, wherein the VH comprises CDRs 1, 2, 3 with the amino acid sequences set forth in SEQ ID NOs: 1165, 1166, and 1167, respectively, and the VL comprises CDRs 1, 2, 3 with the amino acid sequences set forth in SEQ ID NOs: 1168, 1169, and 1170, respectively.

32. The antibody or antigen-binding fragment thereof of claim 1, wherein the VH comprises CDRs 1, 2, 3 with the amino acid sequences set forth in SEQ ID NOs: 1189, 1190, and 1191, respectively, and the VL comprises CDRs 1, 2, 3 with the amino acid sequences set forth in SEQ ID NOs: 1192, 1193, and 1194, respectively.

33. The antibody or antigen-binding fragment thereof of claim 1, wherein the VH comprises CDRs 1, 2, 3 with the amino acid sequences set forth in SEQ ID NOs: 1213, 1214, and 1215, respectively, and the VL comprises CDRs 1, 2, 3 with the amino acid sequences set forth in SEQ ID NOs: 1216, 1217, and 1218, respectively.

34. The antibody or antigen-binding fragment thereof of claim 1, wherein the VH comprises CDRs 1, 2, 3 with the amino acid sequences set forth in SEQ ID NOs: 1237, 1238, and 1239, respectively, and the VL comprises CDRs 1, 2, 3 with the amino acid sequences set forth in SEQ ID NOs: 1240, 1241, and 1242, respectively.

35. The antibody or antigen-binding fragment thereof of claim 1, wherein the VH comprises CDRs 1, 2, 3 with the amino acid sequences set forth in SEQ ID NOs: 1261, 1262, and 1263, respectively, and the VL comprises CDRs 1, 2, 3 with the amino acid sequences set forth in SEQ ID NOs: 1264, 1265, and 1266, respectively.

36. The antibody or antigen-binding fragment thereof of claim 1, wherein the VH comprises CDRs 1, 2, 3 with the amino acid sequences set forth in SEQ ID NOs: 1285, 1286, and 1287, respectively, and the VL comprises CDRs 1, 2, 3 with the amino acid sequences set forth in SEQ ID NOs: 1288, 1289, and 1290, respectively.

37. The antibody or antigen-binding fragment thereof of claim 1, wherein the VH comprises CDRs 1, 2, 3 with the amino acid sequences set forth in SEQ ID NOs: 1309, 1310, and 1311, respectively, and the VL comprises CDRs 1, 2, 3 with the amino acid sequences set forth in SEQ ID NOs: 1312, 1313, and 1314, respectively.

38. The antibody or antigen-binding fragment thereof of claim 1, wherein the VH comprises CDRs 1, 2, 3 with the amino acid sequences set forth in SEQ ID NOs: 1333, 1334, and 1335, respectively, and the VL comprises CDRs 1, 2, 3 with the amino acid sequences set forth in SEQ ID NOs: 1336, 1337, and 1338, respectively.

39. The antibody or antigen-binding fragment thereof of claim 1, wherein the VH comprises CDRs 1, 2, 3 with the amino acid sequences set forth in SEQ ID NOs: 1357, 1358, and 1359, respectively, and the VL comprises CDRs 1, 2, 3 with the amino acid sequences set forth in SEQ ID NOs: 1360, 1361, and 1362, respectively.

40. The antibody or antigen-binding fragment thereof of claim 1, wherein the VH comprises CDRs 1, 2, 3 with the amino acid sequences set forth in SEQ ID NOs: 1381, 1382, and 1383, respectively, and the VL comprises CDRs 1, 2, 3 with the amino acid sequences set forth in SEQ ID NOs: 1384, 1385, and 1386, respectively.

41. The antibody or antigen-binding fragment thereof of claim 1, wherein the VH comprises CDRs 1, 2, 3 with the amino acid sequences set forth in SEQ ID NOs: 1405, 1406, and 1407, respectively, and the VL comprises CDRs 1, 2, 3 with the amino acid sequences set forth in SEQ ID NOs: 1408, 1409, and 1410, respectively.

42. The antibody or antigen-binding fragment thereof of claim 1, wherein the VH comprises CDRs 1, 2, 3 with the amino acid sequences set forth in SEQ ID NOs: 1429, 1430, and 1431, respectively, and the VL comprises CDRs 1, 2, 3 with the amino acid sequences set forth in SEQ ID NOs: 1432, 1433, and 1434, respectively.

43. The antibody or antigen-binding fragment thereof of claim 1, wherein the VH comprises CDRs 1, 2, 3 with the amino acid sequences set forth in SEQ ID NOs: 1453, 1454, and 1455, respectively, and the VL comprises CDRs 1, 2, 3 with the amino acid sequences set forth in SEQ ID NOs: 1456, 1457, and 1458, respectively.

44. The antibody or antigen-binding fragment thereof of claim 1, wherein the VH comprises CDRs 1, 2, 3 with the amino acid sequences set forth in SEQ ID NOs: 1477, 1478, and 1479, respectively, and the VL comprises CDRs 1, 2, 3 with the amino acid sequences set forth in SEQ ID NOs: 1480, 1481, and 1482, respectively.

45. The antibody or antigen-binding fragment thereof of claim 1, wherein the VH comprises CDRs 1, 2, 3 with the amino acid sequences set forth in SEQ ID NOs: 1501, 1502, and 1503, respectively, and the VL comprises CDRs 1, 2, 3 with the amino acid sequences set forth in SEQ ID NOs: 1504, 1505, and 1506, respectively.

46. The antibody or antigen-binding fragment thereof of claim 1, wherein the VH comprises CDRs 1, 2, 3 with the amino acid sequences set forth in SEQ ID NOs: 1525, 1526, and 1527, respectively, and the VL comprises CDRs 1, 2, 3 with the amino acid sequences set forth in SEQ ID NOs: 1528, 1529, and 1530, respectively.

47. The antibody or antigen-binding fragment thereof of claim 1, wherein the VH comprises CDRs 1, 2, 3 with the amino acid sequences set forth in SEQ ID NOs: 1549, 1550, and 1551, respectively, and the VL comprises CDRs 1, 2, 3 with the amino acid sequences set forth in SEQ ID NOs: 1552, 1553, and 1554, respectively.

48. The antibody or antigen-binding fragment thereof of claim 1, wherein the VH comprises CDRs 1, 2, 3 with the amino acid sequences set forth in SEQ ID NOs: 1573, 1574, and 1575, respectively, and the VL comprises CDRs 1, 2, 3 with the amino acid sequences set forth in SEQ ID NOs: 1576, 1577, and 1578, respectively.

49. The antibody or antigen-binding fragment thereof of claim 1, wherein the VH comprises CDRs 1, 2, 3 with the amino acid sequences set forth in SEQ ID NOs: 1597, 1598, and 1599, respectively, and the VL comprises CDRs 1, 2, 3 with the amino acid sequences set forth in SEQ ID NOs: 1600, 1601, and 1602, respectively.

50. The antibody or antigen-binding fragment thereof of claim 1, wherein the VH comprises CDRs 1, 2, 3 with the amino acid sequences set forth in SEQ ID NOs: 1621, 1622, and 1623, respectively, and the VL comprises CDRs 1, 2, 3 with the amino acid sequences set forth in SEQ ID NOs: 1624, 1625, and 1626, respectively.

51. The antibody or antigen-binding fragment thereof of claim 1, wherein the VH comprises CDRs 1, 2, 3 with the amino acid sequences set forth in SEQ ID NOs: 1645, 1646, and 1647, respectively, and the VL comprises CDRs 1, 2, 3 with the amino acid sequences set forth in SEQ ID NOs: 1648, 1649, and 1650, respectively.

52. The antibody or antigen-binding fragment thereof of claim 1, wherein the VH comprises CDRs 1, 2, 3 with the amino acid sequences set forth in SEQ ID NOs: 1669, 1670, and 1671, respectively, and the VL comprises CDRs 1, 2, 3 with the amino acid sequences set forth in SEQ ID NOs: 1672, 1673, and 1674, respectively.

53. The antibody or antigen-binding fragment thereof of any one of claims 1-52, wherein the antibody or antigen-binding fragment thereof specifically binds to human LILRB1 and/or human LILRB2.

54. The antibody or antigen-binding fragment thereof of any one of claims 1-53, wherein the antibody or antigen-binding fragment thereof specifically binds to monkey LILRB1 and/or monkey LILRB2.

55. The antibody or antigen-binding fragment thereof of any one of claims 1-54, wherein the antibody or antigen-binding fragment thereof can block the interaction between LILRB1 and MHC Class I molecules (e.g., HLA-G), or the interaction between LILRB2 and MHC Class I molecules (e.g., HLA-G).

56. The antibody or antigen-binding fragment thereof of any one of claims 1-55, wherein the antibody or antigen-binding fragment thereof is a humanized antibody or antigen-binding fragment thereof.

57. The antibody or antigen-binding fragment thereof of any one of claims 1-56, wherein the antibody or antigen-binding fragment thereof is a single-chain variable fragment (scFV) or a multi-specific antibody (e.g., a bispecific antibody).

58. The antibody or antigen-binding fragment thereof of any one of claims 1-57, wherein the antibody or antigen-binding fragment thereof specifically binds to LILRB3 (e.g., human LILRB3).

59. A nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 7, 8, and 9, respectively, and wherein the VH, when paired with a light chain variable region (VL) comprising the amino acid sequence set forth in SEQ ID NO: 752 binds to LILRB1 and/or LILRB2;
(2) an immunoglobulin light chain or a fragment thereof comprising a VL comprising CDRs 1, 2, and 3 comprising the amino acid sequences set forth in SEQ ID NOs: 10, 11, and 12, respectively, and wherein the VL, when paired with a VH comprising the amino acid sequence set forth in SEQ ID NO: 751 binds to LILRB1 and/or LILRB2;
(3) an immunoglobulin heavy chain or a fragment thereof comprising a VH comprising CDRs 1, 2, and 3 comprising the amino acid sequences set forth in SEQ ID NOs: 37, 38, and 39, respectively, and wherein the VH, when paired with a VL comprising the amino acid sequence set forth in SEQ ID NO: 754 binds to LILRB1 and/or LILRB2;
(4) an immunoglobulin light chain or a fragment thereof comprising a VL comprising CDRs 1, 2, and 3 comprising the amino acid sequences set forth in SEQ ID NOs: 40, 41, and 42, respectively, and wherein the VL, when paired with a VH comprising the amino acid sequence set forth in SEQ ID NO: 753 binds to LILRB1 and/or LILRB2;
(5) an immunoglobulin heavy chain or a fragment thereof comprising a VH comprising CDRs 1, 2, and 3 comprising the amino acid sequences set forth in SEQ ID NOs: 67, 68, and 69, respectively, and wherein the VH, when paired with a VL comprising the amino acid sequence set forth in SEQ ID NO: 756 binds to LILRB1 and/or LILRB2;
(6) an immunoglobulin light chain or a fragment thereof comprising a VL comprising CDRs 1, 2, and 3 comprising the amino acid sequences set forth in SEQ ID NOs: 70, 71, and 72, respectively, and wherein the VL, when paired with a VH comprising the amino acid sequence set forth in SEQ ID NO: 755 binds to LILRB1 and/or LILRB2;
(7) an immunoglobulin heavy chain or a fragment thereof comprising a VH comprising CDRs 1, 2, and 3 comprising the amino acid sequences set forth in SEQ ID NOs: 97, 98, and 99, respectively, and wherein the VH, when paired with a VL comprising the amino acid sequence set forth in SEQ ID NO: 758 binds to LILRB1 and/or LILRB2;
(8) an immunoglobulin light chain or a fragment thereof comprising a VL comprising CDRs 1, 2, and 3 comprising the amino acid sequences set forth in SEQ ID NOs: 100, 101, and 102, respectively, and wherein the VL, when paired with a VH comprising the amino acid sequence set forth in SEQ ID NO: 757 binds to LILRB1 and/or LILRB2;
(9) an immunoglobulin heavy chain or a fragment thereof comprising a VH comprising CDRs 1, 2, and 3 comprising the amino acid sequences set forth in SEQ ID NOs: 127, 128, and 129, respectively, and wherein the VH, when paired with a VL comprising the amino acid sequence set forth in SEQ ID NO: 760 binds to LILRB1 and/or LILRB2;
(10) an immunoglobulin light chain or a fragment thereof comprising a VL comprising CDRs 1, 2, and 3 comprising the amino acid sequences set forth in SEQ ID NOs: 130, 131, and 132, respectively, and wherein the VL, when paired with a VH comprising the amino acid sequence set forth in SEQ ID NO: 759 binds to LILRB1 and/or LILRB2;
(11) an immunoglobulin heavy chain or a fragment thereof comprising a VH comprising CDRs 1, 2, and 3 comprising the amino acid sequences set forth in SEQ ID NOs: 157, 158, and 159, respectively, and wherein the VH, when paired with a VL comprising the amino acid sequence set forth in SEQ ID NO: 762 binds to LILRB1 and/or LILRB2;
(12) an immunoglobulin light chain or a fragment thereof comprising a VL comprising CDRs 1, 2, and 3 comprising the amino acid sequences set forth in SEQ ID NOs: 160, 161, and 162, respectively, and wherein the VL, when paired with a VH comprising the amino acid sequence set forth in SEQ ID NO: 761 binds to LILRB1 and/or LILRB2;
(13) an immunoglobulin heavy chain or a fragment thereof comprising a VH comprising CDRs 1, 2, and 3 comprising the amino acid sequences set forth in SEQ ID NOs: 187, 188, and 189, respectively, and wherein the VH, when paired with a VL comprising the amino acid sequence set forth in SEQ ID NO: 764 binds to LILRB1 and/or LILRB2;
(14) an immunoglobulin light chain or a fragment thereof comprising a VL comprising CDRs 1, 2, and 3 comprising the amino acid sequences set forth in SEQ ID NOs: 190, 191, and 192, respectively, and wherein the VL, when paired with a VH comprising the amino acid sequence set forth in SEQ ID NO: 763 binds to LILRB1 and/or LILRB2;
(15) an immunoglobulin heavy chain or a fragment thereof comprising a VH comprising CDRs 1, 2, and 3 comprising the amino acid sequences set forth in SEQ ID NOs: 217, 218, and 219, respectively, and wherein the VH, when paired with a VL comprising the amino acid sequence set forth in SEQ ID NO: 766 binds to LILRB1 and/or LILRB2;
(16) an immunoglobulin light chain or a fragment thereof comprising a VL comprising CDRs 1, 2, and 3 comprising the amino acid sequences set forth in SEQ ID NOs: 220, 221, and 222, respectively, and wherein the VL, when paired with a VH comprising the amino acid sequence set forth in SEQ ID NO: 765 binds to LILRB1 and/or LILRB2;
(17) an immunoglobulin heavy chain or a fragment thereof comprising a VH comprising CDRs 1, 2, and 3 comprising the amino acid sequences set forth in SEQ ID NOs: 247, 248, and 249, respectively, and wherein the VH, when paired with a VL comprising the amino acid sequence set forth in SEQ ID NO: 768 binds to LILRB1 and/or LILRB2;
(18) an immunoglobulin light chain or a fragment thereof comprising a VL comprising CDRs 1, 2, and 3 comprising the amino acid sequences set forth in SEQ ID NOs: 250, 251, and 252, respectively, and wherein the VL, when paired with a VH comprising the amino acid sequence set forth in SEQ ID NO: 767 binds to LILRB1 and/or LILRB2;
(19) an immunoglobulin heavy chain or a fragment thereof comprising a VH comprising CDRs 1, 2, and 3 comprising the amino acid sequences set forth in SEQ ID NOs: 277, 278, and 279, respectively, and wherein the VH, when paired with a VL comprising the amino acid sequence set forth in SEQ ID NO: 770 binds to LILRB1 and/or LILRB2;
(20) an immunoglobulin light chain or a fragment thereof comprising a VL comprising CDRs 1, 2, and 3 comprising the amino acid sequences set forth in SEQ ID NOs: 280, 281, and 282, respectively, and wherein the VL, when paired with a VH comprising the amino acid sequence set forth in SEQ ID NO: 769 binds to LILRB1 and/or LILRB2;
(21) an immunoglobulin heavy chain or a fragment thereof comprising a VH comprising CDRs 1, 2, and 3 comprising the amino acid sequences set forth in SEQ ID NOs: 307, 308, and 309, respectively, and wherein the VH, when paired with a VL comprising the amino acid sequence set forth in SEQ ID NO: 772 binds to LILRB1 and/or LILRB2;
(22) an immunoglobulin light chain or a fragment thereof comprising a VL comprising CDRs 1, 2, and 3 comprising the amino acid sequences set forth in SEQ ID NOs: 310, 311, and 312, respectively, and wherein the VL, when paired with a VH comprising the amino acid sequence set forth in SEQ ID NO: 771 binds to LILRB1 and/or LILRB2;
(23) an immunoglobulin heavy chain or a fragment thereof comprising a VH comprising CDRs 1, 2, and 3 comprising the amino acid sequences set forth in SEQ ID NOs: 337, 338, and 339, respectively, and wherein the VH, when paired with a VL comprising the amino acid sequence set forth in SEQ ID NO: 774 binds to LILRB1 and/or LILRB2;
(24) an immunoglobulin light chain or a fragment thereof comprising a VL comprising CDRs 1, 2, and 3 comprising the amino acid sequences set forth in SEQ ID NOs: 340, 341, and 342, respectively, and wherein the VL, when paired with a VH comprising the amino acid sequence set forth in SEQ ID NO: 773 binds to LILRB1 and/or LILRB2;
(25) an immunoglobulin heavy chain or a fragment thereof comprising a VH comprising CDRs 1, 2, and 3 comprising the amino acid sequences set forth in SEQ ID NOs: 367, 368, and 369, respectively, and wherein the VH, when paired with a VL comprising the amino acid sequence set forth in SEQ ID NO: 776 binds to LILRB1 and/or LILRB2;
(26) an immunoglobulin light chain or a fragment thereof comprising a VL comprising CDRs 1, 2, and 3 comprising the amino acid sequences set forth in SEQ ID NOs: 370, 371, and 372, respectively, and wherein the VL, when paired with a VH comprising the amino acid sequence set forth in SEQ ID NO: 775 binds to LILRB1 and/or LILRB2;
(27) an immunoglobulin heavy chain or a fragment thereof comprising a VH comprising CDRs 1, 2, and 3 comprising the amino acid sequences set forth in SEQ ID NOs: 397, 398, and 399, respectively, and wherein the VH, when paired with a VL comprising the amino acid sequence set forth in SEQ ID NO: 778 binds to LILRB1 and/or LILRB2;
(28) an immunoglobulin light chain or a fragment thereof comprising a VL comprising CDRs 1, 2, and 3 comprising the amino acid sequences set forth in SEQ ID NOs: 400, 401, and 402, respectively, and wherein the VL, when paired with a VH comprising the amino acid sequence set forth in SEQ ID NO: 777 binds to LILRB1 and/or LILRB2;
(29) an immunoglobulin heavy chain or a fragment thereof comprising a VH comprising CDRs 1, 2, and 3 comprising the amino acid sequences set forth in SEQ ID NOs: 427, 428, and 429, respectively, and wherein the VH, when paired with a VL comprising the amino acid sequence set forth in SEQ ID NO: 780 binds to LILRB1 and/or LILRB2;
(30) an immunoglobulin light chain or a fragment thereof comprising a VL comprising CDRs 1, 2, and 3 comprising the amino acid sequences set forth in SEQ ID NOs: 430, 431, and 432, respectively, and wherein the VL, when paired with a VH comprising the amino acid sequence set forth in SEQ ID NO: 779 binds to LILRB1 and/or LILRB2;
(31) an immunoglobulin heavy chain or a fragment thereof comprising a VH comprising CDRs 1, 2, and 3 comprising the amino acid sequences set forth in SEQ ID NOs: 457, 458, and 459, respectively, and wherein the VH, when paired with a VL comprising the amino acid sequence set forth in SEQ ID NO: 782 binds to LILRB1 and/or LILRB2;
(32) an immunoglobulin light chain or a fragment thereof comprising a VL comprising CDRs 1, 2, and 3 comprising the amino acid sequences set forth in SEQ ID NOs: 460, 461, and 462, respectively, and wherein the VL, when paired with a VH comprising the amino acid sequence set forth in SEQ ID NO: 781 binds to LILRB1 and/or LILRB2;
(33) an immunoglobulin heavy chain or a fragment thereof comprising a VH comprising CDRs 1, 2, and 3 comprising the amino acid sequences set forth in SEQ ID NOs: 487, 488, and 489, respectively, and wherein the VH, when paired with a VL comprising the amino acid sequence set forth in SEQ ID NO: 784 binds to LILRB1 and/or LILRB2;
(34) an immunoglobulin light chain or a fragment thereof comprising a VL comprising CDRs 1, 2, and 3 comprising the amino acid sequences set forth in SEQ ID NOs: 490, 491, and 492, respectively, and wherein the VL, when paired with a VH comprising the amino acid sequence set forth in SEQ ID NO: 783 binds to LILRB1 and/or LILRB2;
(35) an immunoglobulin heavy chain or a fragment thereof comprising a VH comprising CDRs 1, 2, and 3 comprising the amino acid sequences set forth in SEQ ID NOs: 517, 518, and 519, respectively, and wherein the VH, when paired with a VL comprising the amino acid sequence set forth in SEQ ID NO: 786 binds to LILRB1 and/or LILRB2;
(36) an immunoglobulin light chain or a fragment thereof comprising a VL comprising CDRs 1, 2, and 3 comprising the amino acid sequences set forth in SEQ ID NOs: 520, 521, and 522, respectively, and wherein the VL, when paired with a VH comprising the amino acid sequence set forth in SEQ ID NO: 785 binds to LILRB1 and/or LILRB2;
(37) an immunoglobulin heavy chain or a fragment thereof comprising a VH comprising CDRs 1, 2, and 3 comprising the amino acid sequences set forth in SEQ ID NOs: 547, 548, and 549, respectively, and wherein the VH, when paired with a VL comprising the amino acid sequence set forth in SEQ ID NO: 788 binds to LILRB1 and/or LILRB2;
(38) an immunoglobulin light chain or a fragment thereof comprising a VL comprising CDRs 1, 2, and 3 comprising the amino acid sequences set forth in SEQ ID NOs: 550, 551, and 552, respectively, and wherein the VL, when paired with a VH comprising the amino acid sequence set forth in SEQ ID NO: 787 binds to LILRB1 and/or LILRB2;
(39) an immunoglobulin heavy chain or a fragment thereof comprising a VH comprising CDRs 1, 2, and 3 comprising the amino acid sequences set forth in SEQ ID NOs: 577, 578, and 579, respectively, and wherein the VH, when paired with a VL comprising the amino acid sequence set forth in SEQ ID NO: 790 binds to LILRB1 and/or LILRB2;
(40) an immunoglobulin light chain or a fragment thereof comprising a VL comprising CDRs 1, 2, and 3 comprising the amino acid sequences set forth in SEQ ID NOs: 580, 581, and 582, respectively, and wherein the VL, when paired with a VH comprising the amino acid sequence set forth in SEQ ID NO: 789 binds to LILRB1 and/or LILRB2;
(41) an immunoglobulin heavy chain or a fragment thereof comprising a VH comprising CDRs 1, 2, and 3 comprising the amino acid sequences set forth in SEQ ID NOs: 607, 608, and 609, respectively, and wherein the VH, when paired with a VL comprising the amino acid sequence set forth in SEQ ID NO: 792 binds to LILRB1 and/or LILRB2;
(42) an immunoglobulin light chain or a fragment thereof comprising a VL comprising CDRs 1, 2, and 3 comprising the amino acid sequences set forth in SEQ ID NOs: 610, 611, and 612, respectively, and wherein the VL, when paired with a VH comprising the amino acid sequence set forth in SEQ ID NO: 791 binds to LILRB1 and/or LILRB2;
(43) an immunoglobulin heavy chain or a fragment thereof comprising a VH comprising CDRs 1, 2, and 3 comprising the amino acid sequences set forth in SEQ ID NOs: 637, 638, and 639, respectively, and wherein the VH, when paired with a VL comprising the amino acid sequence set forth in SEQ ID NO: 794 binds to LILRB1 and/or LILRB2;
(44) an immunoglobulin light chain or a fragment thereof comprising a VL comprising CDRs 1, 2, and 3 comprising the amino acid sequences set forth in SEQ ID NOs: 640, 641, and 642, respectively, and wherein the VL, when paired with a VH comprising the amino acid sequence set forth in SEQ ID NO: 793 binds to LILRB1 and/or LILRB2;
(45) an immunoglobulin heavy chain or a fragment thereof comprising a VH comprising CDRs 1, 2, and 3 comprising the amino acid sequences set forth in SEQ ID NOs: 667, 668, and 669, respectively, and wherein the VH, when paired with a VL comprising the amino acid sequence set forth in SEQ ID NO: 796 binds to LILRB1 and/or LILRB2;
(46) an immunoglobulin light chain or a fragment thereof comprising a VL comprising CDRs 1, 2, and 3 comprising the amino acid sequences set forth in SEQ ID NOs: 670, 671, and 672, respectively, and wherein the VL, when paired with a VH comprising the amino acid sequence set forth in SEQ ID NO: 795 binds to LILRB1 and/or LILRB2;
(47) an immunoglobulin heavy chain or a fragment thereof comprising a VH comprising CDRs 1, 2, and 3 comprising the amino acid sequences set forth in SEQ ID NOs: 697, 698, and 699, respectively, and wherein the VH, when paired with a VL comprising the amino acid sequence set forth in SEQ ID NO: 798 binds to LILRB1 and/or LILRB2;
(48) an immunoglobulin light chain or a fragment thereof comprising a VL comprising CDRs 1, 2, and 3 comprising the amino acid sequences set forth in SEQ ID NOs: 700, 701, and 702, respectively, and wherein the VL, when paired with a VH comprising the amino acid sequence set forth in SEQ ID NO: 797 binds to LILRB1 and/or LILRB2;
(49) an immunoglobulin heavy chain or a fragment thereof comprising a VH comprising CDRs 1, 2, and 3 comprising the amino acid sequences set forth in SEQ ID NOs: 727, 728, and 729, respectively, and wherein the VH, when paired with a VL comprising the amino acid sequence set forth in SEQ ID NO: 800 binds to LILRB1 and/or LILRB2;
(50) an immunoglobulin light chain or a fragment thereof comprising a VL comprising CDRs 1, 2, and 3 comprising the amino acid sequences set forth in SEQ ID NOs: 730, 731, and 732, respectively, and wherein the VL, when paired with a VH comprising the amino acid sequence set forth in SEQ ID NO: 799 binds to LILRB1 and/or LILRB2;
(51) an immunoglobulin heavy chain or a fragment thereof comprising a VH comprising CDRs 1, 2, and 3 comprising the amino acid sequences set forth in SEQ ID NOs: 1069, 1070, and 1071, respectively, and wherein the VH, when paired with a VL comprising the amino acid sequence set forth in SEQ ID NO: 988 binds to LILRB1 and/or LILRB2;
(52) an immunoglobulin light chain or a fragment thereof comprising a VL comprising CDRs 1, 2, and 3 comprising the amino acid sequences set forth in SEQ ID NOs: 1072, 1073, and 1074, respectively, and wherein the VL, when paired with a VH comprising the amino acid sequence set forth in SEQ ID NO: 987 binds to LILRB1 and/or LILRB2;
(53) an immunoglobulin heavy chain or a fragment thereof comprising a VH comprising CDRs 1, 2, and 3 comprising the amino acid sequences set forth in SEQ ID NOs: 1093, 1094, and 1095, respectively, and wherein the VH, when paired with a VL comprising the amino acid sequence set forth in SEQ ID NO: 988 binds to LILRB1 and/or LILRB2;
(54) an immunoglobulin light chain or a fragment thereof comprising a VL comprising CDRs 1, 2, and 3 comprising the amino acid sequences set forth in SEQ ID NOs: 1096, 1097, and 1098, respectively, and wherein the VL, when paired with a VH comprising the amino acid sequence set forth in SEQ ID NO: 1687 binds to LILRB1 and/or LILRB2;
(55) an immunoglobulin heavy chain or a fragment thereof comprising a VH comprising CDRs 1, 2, and 3 comprising the amino acid sequences set forth in SEQ ID NOs: 1117, 1118, and 1119, respectively, and wherein the VH, when paired with a VL comprising the amino acid sequence set forth in SEQ ID NO: 988 binds to LILRB1 and/or LILRB2;
(56) an immunoglobulin light chain or a fragment thereof comprising a VL comprising CDRs 1, 2, and 3 comprising the amino acid sequences set forth in SEQ ID NOs: 1120, 1121, and 1122, respectively, and wherein the VL, when paired with a VH comprising the amino acid sequence set forth in SEQ ID NO: 1688 binds to LILRB1 and/or LILRB2;
(57) an immunoglobulin heavy chain or a fragment thereof comprising a VH comprising CDRs 1, 2, and 3 comprising the amino acid sequences set forth in SEQ ID NOs: 1141, 1142, and 1143, respectively, and wherein the VH, when paired with a VL comprising the amino acid sequence set forth in SEQ ID NO: 989 binds to LILRB1 and/or LILRB2;
(58) an immunoglobulin light chain or a fragment thereof comprising a VL comprising CDRs 1, 2, and 3 comprising the amino acid sequences set forth in SEQ ID NOs: 1144, 1145, and 1146, respectively, and wherein the VL, when paired with a VH comprising the amino acid sequence set forth in SEQ ID NO: 987 binds to LILRB1 and/or LILRB2;
(59) an immunoglobulin heavy chain or a fragment thereof comprising a VH comprising CDRs 1, 2, and 3 comprising the amino acid sequences set forth in SEQ ID NOs: 1165, 1166, and 1167, respectively, and wherein the VH, when paired with a VL comprising the amino acid sequence set forth in SEQ ID NO: 990 binds to LILRB1 and/or LILRB2;
(60) an immunoglobulin light chain or a fragment thereof comprising a VL comprising CDRs 1, 2, and 3 comprising the amino acid sequences set forth in SEQ ID NOs: 1168, 1169, and 1170, respectively, and wherein the VL, when paired with a VH comprising the amino acid sequence set forth in SEQ ID NO: 987 binds to LILRB1 and/or LILRB2;
(61) an immunoglobulin heavy chain or a fragment thereof comprising a VH comprising CDRs 1, 2, and 3 comprising the amino acid sequences set forth in SEQ ID NOs: 1189, 1190, and 1191, respectively, and wherein the VH, when paired with a VL comprising the amino acid sequence set forth in SEQ ID NO: 991 binds to LILRB1 and/or LILRB2;
(62) an immunoglobulin light chain or a fragment thereof comprising a VL comprising CDRs 1, 2, and 3 comprising the amino acid sequences set forth in SEQ ID NOs: 1192, 1193, and 1194, respectively, and wherein the VL, when paired with a VH comprising the amino acid sequence set forth in SEQ ID NO: 987 binds to LILRB1 and/or LILRB2;
(63) an immunoglobulin heavy chain or a fragment thereof comprising a VH comprising CDRs 1, 2, and 3 comprising the amino acid sequences set forth in SEQ ID NOs: 1213, 1214, and 1215, respectively, and wherein the VH, when paired with a VL comprising the amino acid sequence set forth in SEQ ID NO: 992 binds to LILRB1 and/or LILRB2;
(64) an immunoglobulin light chain or a fragment thereof comprising a VL comprising CDRs 1, 2, and 3 comprising the amino acid sequences set forth in SEQ ID NOs: 1216, 1217, and 1218, respectively, and wherein the VL, when paired with a VH comprising the amino acid sequence set forth in SEQ ID NO: 987 binds to LILRB1 and/or LILRB2;
(65) an immunoglobulin heavy chain or a fragment thereof comprising a VH comprising CDRs 1, 2, and 3 comprising the amino acid sequences set forth in SEQ ID NOs: 1237, 1238, and 1239, respectively, and wherein the VH, when paired with a VL comprising the amino acid sequence set forth in SEQ ID NO: 1020 binds to LILRB1 and/or LILRB2;
(66) an immunoglobulin light chain or a fragment thereof comprising a VL comprising CDRs 1, 2, and 3 comprising the amino acid sequences set forth in SEQ ID NOs: 1240, 1241, and 1242, respectively, and wherein the VL, when paired with a VH comprising the amino acid sequence set forth in SEQ ID NO: 1019 binds to LILRB1 and/or LILRB2;
(67) an immunoglobulin heavy chain or a fragment thereof comprising a VH comprising CDRs 1, 2, and 3 comprising the amino acid sequences set forth in SEQ ID NOs: 1261, 1262, and 1263, respectively, and wherein the VH, when paired with a VL comprising the amino acid sequence set forth in SEQ ID NO: 1022 binds to LILRB1 and/or LILRB2;
(68) an immunoglobulin light chain or a fragment thereof comprising a VL comprising CDRs 1, 2, and 3 comprising the amino acid sequences set forth in SEQ ID NOs: 1264, 1265, and 1266, respectively, and wherein the VL, when paired with a VH comprising the amino acid sequence set forth in SEQ ID NO: 1021 binds to LILRB1 and/or LILRB2;
(69) an immunoglobulin heavy chain or a fragment thereof comprising a VH comprising CDRs 1, 2, and 3 comprising the amino acid sequences set forth in SEQ ID NOs: 1285, 1286, and 1287, respectively, and wherein the VH, when paired with a VL comprising the amino acid sequence set forth in SEQ ID NO: 1024 binds to LILRB1 and/or LILRB2;
(70) an immunoglobulin light chain or a fragment thereof comprising a VL comprising CDRs 1, 2, and 3 comprising the amino acid sequences set forth in SEQ ID NOs: 1288, 1289, and 1290, respectively, and wherein the VL, when paired with a VH comprising the amino acid sequence set forth in SEQ ID NO: 1023 binds to LILRB1 and/or LILRB2;
(71) an immunoglobulin heavy chain or a fragment thereof comprising a VH comprising CDRs 1, 2, and 3 comprising the amino acid sequences set forth in SEQ ID NOs: 1309, 1310, and 1311, respectively, and wherein the VH, when paired with a VL comprising the amino acid sequence set forth in SEQ ID NO: 1026 binds to LILRB1 and/or LILRB2;
(72) an immunoglobulin light chain or a fragment thereof comprising a VL comprising CDRs 1, 2, and 3 comprising the amino acid sequences set forth in SEQ ID NOs: 1312, 1313, and 1314, respectively, and wherein the VL, when paired with a VH comprising the amino acid sequence set forth in SEQ ID NO: 1025 binds to LILRB1 and/or LILRB2;
(73) an immunoglobulin heavy chain or a fragment thereof comprising a VH comprising CDRs 1, 2, and 3 comprising the amino acid sequences set forth in SEQ ID NOs: 1333, 1334, and 1335, respectively, and wherein the VH, when paired with a VL comprising the amino acid sequence set forth in SEQ ID NO: 1028 binds to LILRB1 and/or LILRB2;
(74) an immunoglobulin light chain or a fragment thereof comprising a VL comprising CDRs 1, 2, and 3 comprising the amino acid sequences set forth in SEQ ID NOs: 1336, 1337, and 1338, respectively, and wherein the VL, when paired with a VH comprising the amino acid sequence set forth in SEQ ID NO: 1027 binds to LILRB1 and/or LILRB2;
(75) an immunoglobulin heavy chain or a fragment thereof comprising a VH comprising CDRs 1, 2, and 3 comprising the amino acid sequences set forth in SEQ ID NOs: 1357, 1358, and 1359, respectively, and wherein the VH, when paired with a VL comprising the amino acid sequence set forth in SEQ ID NO: 1030 binds to LILRB1 and/or LILRB2;
(76) an immunoglobulin light chain or a fragment thereof comprising a VL comprising CDRs 1, 2, and 3 comprising the amino acid sequences set forth in SEQ ID NOs: 1360, 1361, and 1362, respectively, and wherein the VL, when paired with a VH comprising the amino acid sequence set forth in SEQ ID NO: 1029 binds to LILRB1 and/or LILRB2;
(77) an immunoglobulin heavy chain or a fragment thereof comprising a VH comprising CDRs 1, 2, and 3 comprising the amino acid sequences set forth in SEQ ID NOs: 1381, 1382, and 1383, respectively, and wherein the VH, when paired with a VL comprising the amino acid sequence set forth in SEQ ID NO: 1032 binds to LILRB1 and/or LILRB2;
(78) an immunoglobulin light chain or a fragment thereof comprising a VL comprising CDRs 1, 2, and 3 comprising the amino acid sequences set forth in SEQ ID NOs: 1384, 1385, and 1386, respectively, and wherein the VL, when paired with a VH comprising the amino acid sequence set forth in SEQ ID NO: 1031 binds to LILRB1 and/or LILRB2;
(79) an immunoglobulin heavy chain or a fragment thereof comprising a VH comprising CDRs 1, 2, and 3 comprising the amino acid sequences set forth in SEQ ID NOs: 1405, 1406, and 1407, respectively, and wherein the VH, when paired with a VL comprising the amino acid sequence set forth in SEQ ID NO: 1034 binds to LILRB1 and/or LILRB2;
(80) an immunoglobulin light chain or a fragment thereof comprising a VL comprising CDRs 1, 2, and 3 comprising the amino acid sequences set forth in SEQ ID NOs: 1408, 1409, and 1410, respectively, and wherein the VL, when paired with a VH comprising the amino acid sequence set forth in SEQ ID NO: 1033 binds to LILRB1 and/or LILRB2;
(81) an immunoglobulin heavy chain or a fragment thereof comprising a VH comprising CDRs 1, 2, and 3 comprising the amino acid sequences set forth in SEQ ID NOs: 1429, 1430, and 1431, respectively, and wherein the VH, when paired with a VL comprising the amino acid sequence set forth in SEQ ID NO: 1036 binds to LILRB1 and/or LILRB2;
(82) an immunoglobulin light chain or a fragment thereof comprising a VL comprising CDRs 1, 2, and 3 comprising the amino acid sequences set forth in SEQ ID NOs: 1432, 1433, and 1434, respectively, and wherein the VL, when paired with a VH comprising the amino acid sequence set forth in SEQ ID NO: 1035 binds to LILRB1 and/or LILRB2;
(83) an immunoglobulin heavy chain or a fragment thereof comprising a VH comprising CDRs 1, 2, and 3 comprising the amino acid sequences set forth in SEQ ID NOs: 1453, 1454, and 1455, respectively, and wherein the VH, when paired with a VL comprising the amino acid sequence set forth in SEQ ID NO: 1038 binds to LILRB1 and/or LILRB2;
(84) an immunoglobulin light chain or a fragment thereof comprising a VL comprising CDRs 1, 2, and 3 comprising the amino acid sequences set forth in SEQ ID NOs: 1456, 1457, and 1458, respectively, and wherein the VL, when paired with a VH comprising the amino acid sequence set forth in SEQ ID NO: 1037 binds to LILRB1 and/or LILRB2;
(85) an immunoglobulin heavy chain or a fragment thereof comprising a VH comprising CDRs 1, 2, and 3 comprising the amino acid sequences set forth in SEQ ID NOs: 1477, 1478, and 1479, respectively, and wherein the VH, when paired with a VL comprising the amino acid sequence set forth in SEQ ID NO: 1040 binds to LILRB1 and/or LILRB2;
(86) an immunoglobulin light chain or a fragment thereof comprising a VL comprising CDRs 1, 2, and 3 comprising the amino acid sequences set forth in SEQ ID NOs: 1480, 1481, and 1482, respectively, and wherein the VL, when paired with a VH comprising the amino acid sequence set forth in SEQ ID NO: 1039 binds to LILRB1 and/or LILRB2;
(87) an immunoglobulin heavy chain or a fragment thereof comprising a VH comprising CDRs 1, 2, and 3 comprising the amino acid sequences set forth in SEQ ID NOs: 1501, 1502, and 1503, respectively, and wherein the VH, when paired with a VL comprising the amino acid sequence set forth in SEQ ID NO: 1042 binds to LILRB1 and/or LILRB2;
(88) an immunoglobulin light chain or a fragment thereof comprising a VL comprising CDRs 1, 2, and 3 comprising the amino acid sequences set forth in SEQ ID NOs: 1504, 1505, and 1506, respectively, and wherein the VL, when paired with a VH comprising the amino acid sequence set forth in SEQ ID NO: 1041 binds to LILRB1 and/or LILRB2;
(89) an immunoglobulin heavy chain or a fragment thereof comprising a VH comprising CDRs 1, 2, and 3 comprising the amino acid sequences set forth in SEQ ID NOs: 1525, 1526, and 1527, respectively, and wherein the VH, when paired with a VL comprising the amino acid sequence set forth in SEQ ID NO: 1044, 1689, or 1690 binds to LILRB1 and/or LILRB2;
(90) an immunoglobulin light chain or a fragment thereof comprising a VL comprising CDRs 1, 2, and 3 comprising the amino acid sequences set forth in SEQ ID NOs: 1528, 1529, and 1530, respectively, and wherein the VL, when paired with a VH comprising the amino acid sequence set forth in SEQ ID NO: 1043 binds to LILRB1 and/or LILRB2;
(91) an immunoglobulin heavy chain or a fragment thereof comprising a VH comprising CDRs 1, 2, and 3 comprising the amino acid sequences set forth in SEQ ID NOs: 1549, 1550, and 1551, respectively, and wherein the VH, when paired with a VL comprising the amino acid sequence set forth in SEQ ID NO: 1046 binds to LILRB1 and/or LILRB2;
(92) an immunoglobulin light chain or a fragment thereof comprising a VL comprising CDRs 1, 2, and 3 comprising the amino acid sequences set forth in SEQ ID NOs: 1552, 1553, and 1554, respectively, and wherein the VL, when paired with a VH comprising the amino acid sequence set forth in SEQ ID NO: 1045 binds to LILRB1 and/or LILRB2;
(93) an immunoglobulin heavy chain or a fragment thereof comprising a VH comprising CDRs 1, 2, and 3 comprising the amino acid sequences set forth in SEQ ID NOs: 1573, 1574, and 1575, respectively, and wherein the VH, when paired with a VL comprising the amino acid sequence set forth in SEQ ID NO: 1048 binds to LILRB1 and/or LILRB2;
(94) an immunoglobulin light chain or a fragment thereof comprising a VL comprising CDRs 1, 2, and 3 comprising the amino acid sequences set forth in SEQ ID NOs: 1576, 1577, and 1578, respectively, and wherein the VL, when paired with a VH comprising the amino acid sequence set forth in SEQ ID NO: 1047 binds to LILRB1 and/or LILRB2;
(95) an immunoglobulin heavy chain or a fragment thereof comprising a VH comprising CDRs 1, 2, and 3 comprising the amino acid sequences set forth in SEQ ID NOs: 1597, 1598, and 1599, respectively, and wherein the VH, when paired with a VL comprising the amino acid sequence set forth in SEQ ID NO: 1050 binds to LILRB1 and/or LILRB2;
(96) an immunoglobulin light chain or a fragment thereof comprising a VL comprising CDRs 1, 2, and 3 comprising the amino acid sequences set forth in SEQ ID NOs: 1600, 1601, and 1602, respectively, and wherein the VL, when paired with a VH comprising the amino acid sequence set forth in SEQ ID NO: 1049 binds to LILRB1 and/or LILRB2;
(97) an immunoglobulin heavy chain or a fragment thereof comprising a VH comprising CDRs 1, 2, and 3 comprising the amino acid sequences set forth in SEQ ID NOs: 1621, 1622, and 1623, respectively, and wherein the VH, when paired with a VL comprising the amino acid sequence set forth in SEQ ID NO: 1052 binds to LILRB1 and/or LILRB2;
(98) an immunoglobulin light chain or a fragment thereof comprising a VL comprising CDRs 1, 2, and 3 comprising the amino acid sequences set forth in SEQ ID NOs: 1624, 1625, and 1626, respectively, and wherein the VL, when paired with a VH comprising the amino acid sequence set forth in SEQ ID NO: 1051 binds to LILRB1 and/or LILRB2;
(99) an immunoglobulin heavy chain or a fragment thereof comprising a VH comprising CDRs 1, 2, and 3 comprising the amino acid sequences set forth in SEQ ID NOs: 1645, 1646, and 1647, respectively, and wherein the VH, when paired with a VL comprising the amino acid sequence set forth in SEQ ID NO: 1054 binds to LILRB1 and/or LILRB2;
(100) an immunoglobulin light chain or a fragment thereof comprising a VL comprising CDRs 1, 2, and 3 comprising the amino acid sequences set forth in SEQ ID NOs: 1648, 1649, and 1650, respectively, and wherein the VL, when paired with a VH comprising the amino acid sequence set forth in SEQ ID NO: 1053 binds to LILRB1 and/or LILRB2;
(101) an immunoglobulin heavy chain or a fragment thereof comprising a VH comprising CDRs 1, 2, and 3 comprising the amino acid sequences set forth in SEQ ID NOs: 1669, 1670, and 1671, respectively, and wherein the VH, when paired with a VL comprising the amino acid sequence set forth in SEQ ID NO: 1056 binds to LILRB1 and/or LILRB2; or
(102) an immunoglobulin light chain or a fragment thereof comprising a VL comprising CDRs 1, 2, and 3 comprising the amino acid sequences set forth in SEQ ID NOs: 1672, 1673, and 1674, respectively, and wherein the VL, when paired with a VH comprising the amino acid sequence set forth in SEQ ID NO: 1055 binds to LILRB1 and/or LILRB2.

60. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 7, 8, and 9, respectively.

61. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 10, 11, and 12, respectively.

62. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 37, 38, and 39, respectively.

63. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 40, 41, and 42, respectively.

64. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 67, 68, and 69, respectively.

65. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 70, 71, and 72, respectively.

66. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 97, 98, and 99, respectively.

67. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 100, 101, and 102, respectively.

68. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 127, 128, and 129, respectively.

69. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 130, 131, and 132, respectively.

70. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 157, 158, and 159, respectively.

71. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 160, 161, and 162, respectively.

72. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 187, 188, and 189, respectively.

73. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 190, 191, and 192, respectively.

74. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 217, 218, and 219, respectively.

75. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 220, 221, and 222, respectively.

76. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 247, 248, and 249, respectively.

77. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 250, 251, and 252, respectively.

78. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 277, 278, and 279 respectively.

79. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 280, 281, and 282, respectively.

80. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 307, 308, and 309, respectively.

81. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 310, 311, and 312, respectively.

82. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 337, 338, and 339, respectively.

83. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 340, 341, and 342, respectively.

84. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 367, 368, and 369, respectively.

85. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 370, 371, and 372, respectively.

86. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 397, 398, and 399, respectively.

87. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 400, 401, and 402, respectively.

88. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 427, 428, and 429, respectively.

89. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 430, 431, and 432, respectively.

90. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 457, 458, and 459, respectively.

91. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 460, 461, and 462, respectively.

92. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 487, 488, and 489, respectively.

93. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 490, 491, and 492, respectively.

94. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 517, 518, and 519, respectively.

95. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 520, 521, and 522, respectively.

96. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 547, 548, and 549, respectively.

97. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 550, 551, and 552, respectively.

98. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 577, 578, and 579, respectively.

99. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 580, 581, and 582, respectively.

100. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 607, 608, and 609, respectively.

101. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 610, 611, and 612, respectively.

102. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 637, 638, and 639, respectively.

103. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 640, 641, and 642, respectively.

104. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 667, 668, and 669, respectively.

105. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 700, 701, and 702, respectively.

106. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 697, 698, and 699, respectively.

107. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 700, 701, and 702, respectively.

108. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 727, 728, and 729, respectively.

109. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 730, 731, and 732, respectively.

110. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1069, 1070, and 1071, respectively.

111. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1072, 1073, and 1074, respectively.

112. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1093, 1094, and 1095, respectively.

113. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1096, 1097, and 1098, respectively.

114. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1117, 1118, and 1119, respectively.

115. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1120, 1121, and 1122, respectively.

116. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1141, 1142, and 1143, respectively.

117. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1144, 1145, and 1146, respectively.

118. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1165, 1166, and 1167, respectively.

119. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1168, 1169, and 1170, respectively.

120. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1189, 1190, and 1191, respectively.

121. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1192, 1193, and 1194, respectively.

122. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1213, 1214, and 1215, respectively.

123. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1216, 1217, and 1218, respectively.

124. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1237, 1238, and 1239, respectively.

125. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1240, 1241, and 1242, respectively.

126. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1261, 1262, and 1263, respectively.

127. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1264, 1265, and 1266, respectively.

128. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1285, 1286, and 1287, respectively.

129. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1288, 1289, and 1290, respectively.

130. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1309, 1310, and 1311, respectively.

131. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1312, 1313, and 1314, respectively.

132. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1333, 1334, and 1335, respectively.

133. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1336, 1337, and 1338, respectively.

134. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1357, 1358, and 1359, respectively.

135. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1360, 1361, and 1362, respectively.

136. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1381, 1382, and 1383, respectively.

137. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1384, 1385, and 1386, respectively.

138. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1405, 1406, and 1407, respectively.

139. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1408, 1409, and 1410, respectively.

140. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1429, 1430, and 1431, respectively.

141. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1432, 1433, and 1434, respectively.

142. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1453, 1454, and 1455, respectively.

143. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1456, 1457, and 1458, respectively.

144. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1477, 1478, and 1479, respectively.

145. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1480, 1481, and 1482, respectively.

146. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1501, 1502, and 1503, respectively.

147. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1504, 1505, and 1506, respectively.

148. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1525, 1526, and 1527, respectively.

149. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1528, 1529, and 1530, respectively.

150. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1549, 1550, and 1551, respectively.

151. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1552, 1553, and 1554, respectively.

152. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1573, 1574, and 1575, respectively.

153. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1576, 1577, and 1578, respectively.

154. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1597, 1598, and 1599, respectively.

155. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1600, 1601, and 1602, respectively.

156. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1621, 1622, and 1623, respectively.

157. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1624, 1625, and 1626, respectively.

158. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1645, 1646, and 1647, respectively.

159. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1648, 1649, and 1650, respectively.

160. The nucleic acid of claim 59, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1669, 1670, and 1671, respectively.

161. The nucleic acid of claim 59, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1672, 1673, and 1674, respectively.

162. The nucleic acid of any one of claims 59-161, wherein the VH when paired with a VL specifically binds to human LILRB1 and/or LILRB2; or the VL when paired with a VH specifically binds to human LILRB1 and/or LILRB2.

163. The nucleic acid of any one of claims 59-162, wherein the immunoglobulin heavy chain or the fragment thereof is a humanized immunoglobulin heavy chain or a fragment thereof, and the immunoglobulin light chain or the fragment thereof is a humanized immunoglobulin light chain or a fragment thereof.

164. The nucleic acid of any one of claims 59-163, wherein the nucleic acid encodes a single-chain variable fragment (scFv) or a multi-specific antibody (e.g., a bispecific antibody).

165. The nucleic acid of any one of claims 59-164, wherein the nucleic acid is cDNA.

166. A vector comprising one or more of the nucleic acids of any one of claims 59-165.

167. A vector comprising two of the nucleic acids of any one of claims 59-165, wherein the vector encodes the VL region and the VH region that together bind to LILRB1 and/or LILRB2.

168. A pair of vectors, wherein each vector comprises one of the nucleic acids of any one of claims 59-165, wherein together the pair of vectors encodes the VL region and the VH region that together bind to LILRB1 and/or LILRB2.

169. A cell comprising the vector of claim 166 or 167, or the pair of vectors of claim 168.

170. The cell of claim 169, wherein the cell is a CHO cell.

171. A cell comprising one or more of the nucleic acids of any one of claims 59-165.

172. A cell comprising two of the nucleic acids of any one of claims 59-165.

173. The cell of claim 172, wherein the two nucleic acids together encode the VL region and the VH region that together bind to LILRB1 and/or LILRB2.

174. A method of producing an antibody or an antigen-binding fragment thereof, the method comprising
(a) culturing the cell of any one of claims 169-173 under conditions sufficient for the cell to produce the antibody or the antigen-binding fragment; and
(b) collecting the antibody or the antigen-binding fragment produced by the cell.

175. An antibody or antigen-binding fragment thereof that binds to LILRB1 and/or LILRB2 comprising a heavy chain variable region (VH) comprising an amino acid sequence that is at least 90% identical to a selected VH sequence, and a light chain variable region (VL) comprising an amino acid sequence that is at least 90% identical to a selected VL sequence, wherein the selected VH sequence and the selected VL sequence are one of the following:
(1) the selected VH sequence is SEQ ID NO: 751, and the selected VL sequence is SEQ ID NO: 752;
(2) the selected VH sequence is SEQ ID NO: 753, and the selected VL sequence is SEQ ID NO: 754;
(3) the selected VH sequence is SEQ ID NO: 755, and the selected VL sequence is SEQ ID NO: 756;
(4) the selected VH sequence is SEQ ID NO: 757, and the selected VL sequence is SEQ ID NO: 758;
(5) the selected VH sequence is SEQ ID NO: 759, and the selected VL sequence is SEQ ID NO: 760;
(6) the selected VH sequence is SEQ ID NO: 761, and the selected VL sequence is SEQ ID NO: 762;
(7) the selected VH sequence is SEQ ID NO: 763, and the selected VL sequence is SEQ ID NO: 764;
(8) the selected VH sequence is SEQ ID NO: 765, and the selected VL sequence is SEQ ID NO: 766;
(9) the selected VH sequence is SEQ ID NO: 767, and the selected VL sequence is SEQ ID NO: 768;
(10) the selected VH sequence is SEQ ID NO: 769, and the selected VL sequence is SEQ ID NO: 770;
(11) the selected VH sequence is SEQ ID NO: 771, and the selected VL sequence is SEQ ID NO: 772;
(12) the selected VH sequence is SEQ ID NO: 773, and the selected VL sequence is SEQ ID NO: 774;
(13) the selected VH sequence is SEQ ID NO: 775, and the selected VL sequence is SEQ ID NO: 776;
(14) the selected VH sequence is SEQ ID NO: 777, and the selected VL sequence is SEQ ID NO: 778;
(15) the selected VH sequence is SEQ ID NO: 779, and the selected VL sequence is SEQ ID NO: 780;
(16) the selected VH sequence is SEQ ID NO: 781, and the selected VL sequence is SEQ ID NO: 782;
(17) the selected VH sequence is SEQ ID NO: 783, and the selected VL sequence is SEQ ID NO: 784;
(18) the selected VH sequence is SEQ ID NO: 785, and the selected VL sequence is SEQ ID NO: 786;
(19) the selected VH sequence is SEQ ID NO: 787, and the selected VL sequence is SEQ ID NO: 788;
(20) the selected VH sequence is SEQ ID NO: 789, and the selected VL sequence is SEQ ID NO: 790;
(21) the selected VH sequence is SEQ ID NO: 791, and the selected VL sequence is SEQ ID NO: 792;
(22) the selected VH sequence is SEQ ID NO: 793, and the selected VL sequence is SEQ ID NO: 794;
(23) the selected VH sequence is SEQ ID NO: 795, and the selected VL sequence is SEQ ID NO: 796;
(24) the selected VH sequence is SEQ ID NO: 797, and the selected VL sequence is SEQ ID NO: 798;
(25) the selected VH sequence is SEQ ID NO: 799, and the selected VL sequence is SEQ ID NO: 800;
(26) the selected VH sequence is SEQ ID NO: 841, and the selected VL sequence is SEQ ID NO: 842;
(27) the selected VH sequence is SEQ ID NO: 843, and the selected VL sequence is SEQ ID NO: 844;
(28) the selected VH sequence is SEQ ID NO: 845, and the selected VL sequence is SEQ ID NO: 846;
(29) the selected VH sequence is SEQ ID NO: 847, and the selected VL sequence is SEQ ID NO: 848;
(30) the selected VH sequence is SEQ ID NO: 849, and the selected VL sequence is SEQ ID NO: 850;
(31) the selected VH sequence is SEQ ID NO: 851, and the selected VL sequence is SEQ ID NO: 852;
(32) the selected VH sequence is SEQ ID NO: 853, and the selected VL sequence is SEQ ID NO: 854;
(33) the selected VH sequence is SEQ ID NO: 855, and the selected VL sequence is SEQ ID NO: 856;
(34) the selected VH sequence is SEQ ID NO: 857, and the selected VL sequence is SEQ ID NO: 858;
(35) the selected VH sequence is SEQ ID NO: 859, and the selected VL sequence is SEQ ID NO: 860;
(36) the selected VH sequence is SEQ ID NO: 861, and the selected VL sequence is SEQ ID NO: 862;
(37) the selected VH sequence is SEQ ID NO: 863, and the selected VL sequence is SEQ ID NO: 864;
(38) the selected VH sequence is SEQ ID NO: 865, and the selected VL sequence is SEQ ID NO: 866;
(39) the selected VH sequence is SEQ ID NO: 867, and the selected VL sequence is SEQ ID NO: 868;
(40) the selected VH sequence is SEQ ID NO: 869, and the selected VL sequence is SEQ ID NO: 870;
(41) the selected VH sequence is SEQ ID NO: 871, and the selected VL sequence is SEQ ID NO: 872;
(42) the selected VH sequence is SEQ ID NO: 873, and the selected VL sequence is SEQ ID NO: 874;
(43) the selected VH sequence is SEQ ID NO: 875, and the selected VL sequence is SEQ ID NO: 876;
(44) the selected VH sequence is SEQ ID NO: 877, and the selected VL sequence is SEQ ID NO: 878;
(45) the selected VH sequence is SEQ ID NO: 879, and the selected VL sequence is SEQ ID NO: 880;
(46) the selected VH sequence is SEQ ID NO: 881, and the selected VL sequence is SEQ ID NO: 882;
(47) the selected VH sequence is SEQ ID NO: 883, and the selected VL sequence is SEQ ID NO: 884;
(48) the selected VH sequence is SEQ ID NO: 885, and the selected VL sequence is SEQ ID NO: 886;
(49) the selected VH sequence is SEQ ID NO: 887, and the selected VL sequence is SEQ ID NO: 888;
(50) the selected VH sequence is SEQ ID NO: 889, and the selected VL sequence is SEQ ID NO: 890;
(51) the selected VH sequence is SEQ ID NO: 891, and the selected VL sequence is SEQ ID NO: 892;
(52) the selected VH sequence is SEQ ID NO: 893, and the selected VL sequence is SEQ ID NO: 894;
(53) the selected VH sequence is SEQ ID NO: 895, and the selected VL sequence is SEQ ID NO: 896;
(54) the selected VH sequence is SEQ ID NO: 897, and the selected VL sequence is SEQ ID NO: 898;
(55) the selected VH sequence is SEQ ID NO: 899, and the selected VL sequence is SEQ ID NO: 900;
(56) the selected VH sequence is SEQ ID NO: 901, and the selected VL sequence is SEQ ID NO: 902;
(57) the selected VH sequence is SEQ ID NO: 903, and the selected VL sequence is SEQ ID NO: 904;
(58) the selected VH sequence is SEQ ID NO: 905, and the selected VL sequence is SEQ ID NO: 906;
(59) the selected VH sequence is SEQ ID NO: 907, and the selected VL sequence is SEQ ID NO: 908;
(60) the selected VH sequence is SEQ ID NO: 909, and the selected VL sequence is SEQ ID NO: 910;
(61) the selected VH sequence is SEQ ID NO: 911, and the selected VL sequence is SEQ ID NO: 912;
(62) the selected VH sequence is SEQ ID NO: 913, and the selected VL sequence is SEQ ID NO: 914;
(63) the selected VH sequence is SEQ ID NO: 915, and the selected VL sequence is SEQ ID NO: 916;
(64) the selected VH sequence is SEQ ID NO: 917, and the selected VL sequence is SEQ ID NO: 918;
(65) the selected VH sequence is SEQ ID NO: 919, and the selected VL sequence is SEQ ID NO: 920;
(66) the selected VH sequence is SEQ ID NO: 921, and the selected VL sequence is SEQ ID NO: 922;
(67) the selected VH sequence is SEQ ID NO: 923, and the selected VL sequence is SEQ ID NO: 924;
(68) the selected VH sequence is SEQ ID NO: 925, and the selected VL sequence is SEQ ID NO: 926;
(69) the selected VH sequence is SEQ ID NO: 927, and the selected VL sequence is SEQ ID NO: 928;
(70) the selected VH sequence is SEQ ID NO: 929, and the selected VL sequence is SEQ ID NO: 930;
(71) the selected VH sequence is SEQ ID NO: 931, and the selected VL sequence is SEQ ID NO: 932;
(72) the selected VH sequence is SEQ ID NO: 933, and the selected VL sequence is SEQ ID NO: 934;
(73) the selected VH sequence is SEQ ID NO: 935, and the selected VL sequence is SEQ ID NO: 936;
(74) the selected VH sequence is SEQ ID NO: 937, and the selected VL sequence is SEQ ID NO: 938;
(75) the selected VH sequence is SEQ ID NO: 939, and the selected VL sequence is SEQ ID NO: 940;
(76) the selected VH sequence is SEQ ID NO: 941, and the selected VL sequence is SEQ ID NO: 942;
(77) the selected VH sequence is SEQ ID NO: 943, and the selected VL sequence is SEQ ID NO: 944;
(78) the selected VH sequence is SEQ ID NO: 945, and the selected VL sequence is SEQ ID NO: 946;
(79) the selected VH sequence is SEQ ID NO: 947, and the selected VL sequence is SEQ ID NO: 948;
(80) the selected VH sequence is SEQ ID NO: 949, and the selected VL sequence is SEQ ID NO: 950;
(81) the selected VH sequence is SEQ ID NO: 951, and the selected VL sequence is SEQ ID NO: 952;
(82) the selected VH sequence is SEQ ID NO: 953, and the selected VL sequence is SEQ ID NO: 954;
(83) the selected VH sequence is SEQ ID NO: 955, and the selected VL sequence is SEQ ID NO: 956;
(84) the selected VH sequence is SEQ ID NO: 957, and the selected VL sequence is SEQ ID NO: 958;
(85) the selected VH sequence is SEQ ID NO: 959, and the selected VL sequence is SEQ ID NO: 960;
(86) the selected VH sequence is SEQ ID NO: 961, and the selected VL sequence is SEQ ID NO: 962;
(87) the selected VH sequence is SEQ ID NO: 963, and the selected VL sequence is SEQ ID NO: 964;
(88) the selected VH sequence is SEQ ID NO: 965, and the selected VL sequence is SEQ ID NO: 966;
(89) the selected VH sequence is SEQ ID NO: 967, and the selected VL sequence is SEQ ID NO: 968;
(90) the selected VH sequence is SEQ ID NO: 969, and the selected VL sequence is SEQ ID NO: 970;
(91) the selected VH sequence is SEQ ID NO: 971, and the selected VL sequence is SEQ ID NO: 972;
(92) the selected VH sequence is SEQ ID NO: 973, and the selected VL sequence is SEQ ID NO: 974;
(93) the selected VH sequence is SEQ ID NO: 975, and the selected VL sequence is SEQ ID NO: 976;
(94) the selected VH sequence is SEQ ID NO: 977, and the selected VL sequence is SEQ ID NO: 978;
(95) the selected VH sequence is SEQ ID NO: 979, and the selected VL sequence is SEQ ID NO: 980, 981, 982, 983, or 984;
(96) the selected VH sequence is SEQ ID NO: 985, and the selected VL sequence is SEQ ID NO: 986;
(97) the selected VH sequence is SEQ ID NO: 987, 1687, or 1688, and the selected VL sequence is SEQ ID NO: 988, 989, 990, 991, or 992;
(98) the selected VH sequence is SEQ ID NO: 993, and the selected VL sequence is SEQ ID NO: 994;
(99) the selected VH sequence is SEQ ID NO: 995, and the selected VL sequence is SEQ ID NO: 996;
(100) the selected VH sequence is SEQ ID NO: 997, and the selected VL sequence is SEQ ID NO: 998;
(101) the selected VH sequence is SEQ ID NO: 999, and the selected VL sequence is SEQ ID NO: 1000, 1001, 1002, 1003, or 1004;
(102) the selected VH sequence is SEQ ID NO: 1005, and the selected VL sequence is SEQ ID NO: 1006;
(103) the selected VH sequence is SEQ ID NO: 1007, and the selected VL sequence is SEQ ID NO: 1008, 1009, or 1010;
(104) the selected VH sequence is SEQ ID NO: 1011, and the selected VL sequence is SEQ ID NO: 1012;
(105) the selected VH sequence is SEQ ID NO: 1013, and the selected VL sequence is SEQ ID NO: 1014;
(106) the selected VH sequence is SEQ ID NO: 1015, and the selected VL sequence is SEQ ID NO: 1016;
(107) the selected VH sequence is SEQ ID NO: 1017, and the selected VL sequence is SEQ ID NO: 1018;
(108) the selected VH sequence is SEQ ID NO: 1019, and the selected VL sequence is SEQ ID NO: 1020;
(109) the selected VH sequence is SEQ ID NO: 1021, and the selected VL sequence is SEQ ID NO: 1022;
(110) the selected VH sequence is SEQ ID NO: 1023, and the selected VL sequence is SEQ ID NO: 1024;
(111) the selected VH sequence is SEQ ID NO: 1025, and the selected VL sequence is SEQ ID NO: 1026;
(112) the selected VH sequence is SEQ ID NO: 1027, and the selected VL sequence is SEQ ID NO: 1028;
(113) the selected VH sequence is SEQ ID NO: 1029, and the selected VL sequence is SEQ ID NO: 1030;
(114) the selected VH sequence is SEQ ID NO: 1031, and the selected VL sequence is SEQ ID NO: 1032;
(115) the selected VH sequence is SEQ ID NO: 1033, and the selected VL sequence is SEQ ID NO: 1034;
(116) the selected VH sequence is SEQ ID NO: 1035, and the selected VL sequence is SEQ ID NO: 1036;
(117) the selected VH sequence is SEQ ID NO: 1037, and the selected VL sequence is SEQ ID NO: 1038;
(118) the selected VH sequence is SEQ ID NO: 1039, and the selected VL sequence is SEQ ID NO: 1040;
(119) the selected VH sequence is SEQ ID NO: 1041, and the selected VL sequence is SEQ ID NO: 1042;
(120) the selected VH sequence is SEQ ID NO: 1043, and the selected VL sequence is SEQ ID NO: 1044, 1689, or 1690;
(121) the selected VH sequence is SEQ ID NO: 1045, and the selected VL sequence is SEQ ID NO: 1046;
(122) the selected VH sequence is SEQ ID NO: 1047, and the selected VL sequence is SEQ ID NO: 1048;
(123) the selected VH sequence is SEQ ID NO: 1049, and the selected VL sequence is SEQ ID NO: 1050;
(124) the selected VH sequence is SEQ ID NO: 1051, and the selected VL sequence is SEQ ID NO: 1052;
(125) the selected VH sequence is SEQ ID NO: 1053, and the selected VL sequence is SEQ ID NO: 1054; and
(126) the selected VH sequence is SEQ ID NO: 1055, and the selected VL sequence is SEQ ID NO: 1056.

176. The antibody or antigen-binding fragment thereof of claim 175, wherein the VH comprises the sequence of SEQ ID NO: 751 and the VL comprises the sequence of SEQ ID NO: 752.

177. The antibody or antigen-binding fragment thereof of claim 175, wherein the VH comprises the sequence of SEQ ID NO: 753 and the VL comprises the sequence of SEQ ID NO: 754.

178. The antibody or antigen-binding fragment thereof of claim 175, wherein the VH comprises the sequence of SEQ ID NO: 755 and the VL comprises the sequence of SEQ ID NO: 756.

179. The antibody or antigen-binding fragment thereof of claim 175, wherein the VH comprises the sequence of SEQ ID NO: 757 and the VL comprises the sequence of SEQ ID NO: 758.

180. The antibody or antigen-binding fragment thereof of claim 175, wherein the VH comprises the sequence of SEQ ID NO: 759 and the VL comprises the sequence of SEQ ID NO: 760.

181. The antibody or antigen-binding fragment thereof of claim 175, wherein the VH comprises the sequence of SEQ ID NO: 761 and the VL comprises the sequence of SEQ ID NO: 762.

182. The antibody or antigen-binding fragment thereof of claim 175, wherein the VH comprises the sequence of SEQ ID NO: 763 and the VL comprises the sequence of SEQ ID NO: 764.

183. The antibody or antigen-binding fragment thereof of claim 175, wherein the VH comprises the sequence of SEQ ID NO: 765 and the VL comprises the sequence of SEQ ID NO: 766.

184. The antibody or antigen-binding fragment thereof of claim 175, wherein the VH comprises the sequence of SEQ ID NO: 767 and the VL comprises the sequence of SEQ ID NO: 768.

185. The antibody or antigen-binding fragment thereof of claim 175, wherein the VH comprises the sequence of SEQ ID NO: 769 and the VL comprises the sequence of SEQ ID NO: 770.

186. The antibody or antigen-binding fragment thereof of claim 175, wherein the VH comprises the sequence of SEQ ID NO: 771 and the VL comprises the sequence of SEQ ID NO: 772.

187. The antibody or antigen-binding fragment thereof of claim 175, wherein the VH comprises the sequence of SEQ ID NO: 773 and the VL comprises the sequence of SEQ ID NO: 774.

188. The antibody or antigen-binding fragment thereof of claim 175, wherein the VH comprises the sequence of SEQ ID NO: 775 and the VL comprises the sequence of SEQ ID NO: 776.

189. The antibody or antigen-binding fragment thereof of claim 175, wherein the VH comprises the sequence of SEQ ID NO: 777 and the VL comprises the sequence of SEQ ID NO: 778.

190. The antibody or antigen-binding fragment thereof of claim 175, wherein the VH comprises the sequence of SEQ ID NO: 779 and the VL comprises the sequence of SEQ ID NO: 780.

191. The antibody or antigen-binding fragment thereof of claim 175, wherein the VH comprises the sequence of SEQ ID NO: 781 and the VL comprises the sequence of SEQ ID NO: 782.

192. The antibody or antigen-binding fragment thereof of claim 175, wherein the VH comprises the sequence of SEQ ID NO: 783 and the VL comprises the sequence of SEQ ID NO: 784.

193. The antibody or antigen-binding fragment thereof of claim 175, wherein the VH comprises the sequence of SEQ ID NO: 785 and the VL comprises the sequence of SEQ ID NO: 786.

194. The antibody or antigen-binding fragment thereof of claim 175, wherein the VH comprises the sequence of SEQ ID NO: 787 and the VL comprises the sequence of SEQ ID NO: 788.

195. The antibody or antigen-binding fragment thereof of claim 175, wherein the VH comprises the sequence of SEQ ID NO: 789 and the VL comprises the sequence of SEQ ID NO: 790.

196. The antibody or antigen-binding fragment thereof of claim 175, wherein the VH comprises the sequence of SEQ ID NO: 791 and the VL comprises the sequence of SEQ ID NO: 792.

197. The antibody or antigen-binding fragment thereof of claim 175, wherein the VH comprises the sequence of SEQ ID NO: 793 and the VL comprises the sequence of SEQ ID NO: 794.

198. The antibody or antigen-binding fragment thereof of claim 175, wherein the VH comprises the sequence of SEQ ID NO: 795 and the VL comprises the sequence of SEQ ID NO: 796.

199. The antibody or antigen-binding fragment thereof of claim 175, wherein the VH comprises the sequence of SEQ ID NO: 797 and the VL comprises the sequence of SEQ ID NO: 798.

200. The antibody or antigen-binding fragment thereof of claim 175, wherein the VH comprises the sequence of SEQ ID NO: 799 and the VL comprises the sequence of SEQ ID NO: 800.

201. The antibody or antigen-binding fragment thereof of claim 175, wherein the VH comprises the sequence of SEQ ID NO: 841 and the VL comprises the sequence of SEQ ID NO: 842.

202. The antibody or antigen-binding fragment thereof of claim 175, wherein the VH comprises the sequence of SEQ ID NO: 843 and the VL comprises the sequence of SEQ ID NO: 844.

203. The antibody or antigen-binding fragment thereof of claim 175, wherein the VH comprises the sequence of SEQ ID NO: 845 and the VL comprises the sequence of SEQ ID NO: 846.

204. The antibody or antigen-binding fragment thereof of claim 175, wherein the VH comprises the sequence of SEQ ID NO: 847 and the VL comprises the sequence of SEQ ID NO: 848.

205. The antibody or antigen-binding fragment thereof of claim 175, wherein the VH comprises the sequence of SEQ ID NO: 849 and the VL comprises the sequence of SEQ ID NO: 850.

206. The antibody or antigen-binding fragment thereof of claim 175, wherein the VH comprises the sequence of SEQ ID NO: 851 and the VL comprises the sequence of SEQ ID NO: 852.

207. The antibody or antigen-binding fragment thereof of claim 175, wherein the VH comprises the sequence of SEQ ID NO: 853 and the VL comprises the sequence of SEQ ID NO: 854.

208. The antibody or antigen-binding fragment thereof of claim 175, wherein the VH comprises the sequence of SEQ ID NO: 855 and the VL comprises the sequence of SEQ ID NO: 856.

209. The antibody or antigen-binding fragment thereof of claim 175, wherein the VH comprises the sequence of SEQ ID NO: 857 and the VL comprises the sequence of SEQ ID NO: 858.

210. The antibody or antigen-binding fragment thereof of claim 175, wherein the VH comprises the sequence of SEQ ID NO: 859 and the VL comprises the sequence of SEQ ID NO: 860.

211. The antibody or antigen-binding fragment thereof of claim 175, wherein the VH comprises the sequence of SEQ ID NO: 861 and the VL comprises the sequence of SEQ ID NO: 862.

212. The antibody or antigen-binding fragment thereof of claim 175, wherein the VH comprises the sequence of SEQ ID NO: 863 and the VL comprises the sequence of SEQ ID NO: 864.

213. The antibody or antigen-binding fragment thereof of claim 175, wherein the VH comprises the sequence of SEQ ID NO: 865 and the VL comprises the sequence of SEQ ID NO: 866.

214. The antibody or antigen-binding fragment thereof of claim 175, wherein the VH comprises the sequence of SEQ ID NO: 867 and the VL comprises the sequence of SEQ ID NO: 868.

215. The antibody or antigen-binding fragment thereof of claim 175, wherein the VH comprises the sequence of SEQ ID NO: 869 and the VL comprises the sequence of SEQ ID NO: 870.

216. The antibody or antigen-binding fragment thereof of claim 175, wherein the VH comprises the sequence of SEQ ID NO: 871 and the VL comprises the sequence of SEQ ID NO: 872.

217. The antibody or antigen-binding fragment thereof of claim 175, wherein the VH comprises the sequence of SEQ ID NO: 873 and the VL comprises the sequence of SEQ ID NO: 874.

218. The antibody or antigen-binding fragment thereof of claim 175, wherein the VH comprises the sequence of SEQ ID NO: 875 and the VL comprises the sequence of SEQ ID NO: 876.

219. The antibody or antigen-binding fragment thereof of claim 175, wherein the VH comprises the sequence of SEQ ID NO: 877 and the VL comprises the sequence of SEQ ID NO: 878.

220. The antibody or antigen-binding fragment thereof of claim 175, wherein the VH comprises the sequence of SEQ ID NO: 879 and the VL comprises the sequence of SEQ ID NO: 880.

221. The antibody or antigen-binding fragment thereof of claim 175, wherein the VH comprises the sequence of SEQ ID NO: 881 and the VL comprises the sequence of SEQ ID NO: 882.

222. The antibody or antigen-binding fragment thereof of claim 175, wherein the VH comprises the sequence of SEQ ID NO: 883 and the VL comprises the sequence of SEQ ID NO: 884.

223. The antibody or antigen-binding fragment thereof of claim 175, wherein the VH comprises the sequence of SEQ ID NO: 885 and the VL comprises the sequence of SEQ ID NO: 886.

224. The antibody or antigen-binding fragment thereof of claim 175, wherein the VH comprises the sequence of SEQ ID NO: 887 and the VL comprises the sequence of SEQ ID NO: 888.

225. The antibody or antigen-binding fragment thereof of claim 175, wherein the VH comprises the sequence of SEQ ID NO: 889 and the VL comprises the sequence of SEQ ID NO: 890.

226. The antibody or antigen-binding fragment thereof of claim 175, wherein the VH comprises the sequence of SEQ ID NO: 891 and the VL comprises the sequence of SEQ ID NO: 892.

227. The antibody or antigen-binding fragment thereof of claim 175, wherein the VH comprises the sequence of SEQ ID NO: 893 and the VL comprises the sequence of SEQ ID NO: 894.

228. The antibody or antigen-binding fragment thereof of claim 175, wherein the VH comprises the sequence of SEQ ID NO: 895 and the VL comprises the sequence of SEQ ID NO: 896.

229. The antibody or antigen-binding fragment thereof of claim 175, wherein the VH comprises the sequence of SEQ ID NO: 897 and the VL comprises the sequence of SEQ ID NO: 898.

230. The antibody or antigen-binding fragment thereof of claim 175, wherein the VH comprises the sequence of SEQ ID NO: 899 and the VL comprises the sequence of SEQ ID NO: 900.

231. The antibody or antigen-binding fragment thereof of claim 175, wherein the VH comprises the sequence of SEQ ID NO: 901 and the VL comprises the sequence of SEQ ID NO: 902.

232. The antibody or antigen-binding fragment thereof of claim 175, wherein the VH comprises the sequence of SEQ ID NO: 903 and the VL comprises the sequence of SEQ ID NO: 904.

233. The antibody or antigen-binding fragment thereof of claim 175, wherein the VH comprises the sequence of SEQ ID NO: 905 and the VL comprises the sequence of SEQ ID NO: 906.

234. The antibody or antigen-binding fragment thereof of claim 175, wherein the VH comprises the sequence of SEQ ID NO: 907 and the VL comprises the sequence of SEQ ID NO: 908.

235. The antibody or antigen-binding fragment thereof of claim 175, wherein the VH comprises the sequence of SEQ ID NO: 909 and the VL comprises the sequence of SEQ ID NO: 910.

236. The antibody or antigen-binding fragment thereof of claim 175, wherein the VH comprises the sequence of SEQ ID NO: 911 and the VL comprises the sequence of SEQ ID NO: 912.

237. The antibody or antigen-binding fragment thereof of claim 175, wherein the VH comprises the sequence of SEQ ID NO: 913 and the VL comprises the sequence of SEQ ID NO: 914.

238. The antibody or antigen-binding fragment thereof of claim 175, wherein the VH comprises the sequence of SEQ ID NO: 915 and the VL comprises the sequence of SEQ ID NO: 916.

239. The antibody or antigen-binding fragment thereof of claim 175, wherein the VH comprises the sequence of SEQ ID NO: 917 and the VL comprises the sequence of SEQ ID NO: 918.

240. The antibody or antigen-binding fragment thereof of claim 175, wherein the VH comprises the sequence of SEQ ID NO: 919 and the VL comprises the sequence of SEQ ID NO: 920.

241. The antibody or antigen-binding fragment thereof of claim 175, wherein the VH comprises the sequence of SEQ ID NO: 921 and the VL comprises the sequence of SEQ ID NO: 922.

242. The antibody or antigen-binding fragment thereof of claim 175, wherein the VH comprises the sequence of SEQ ID NO: 923 and the VL comprises the sequence of SEQ ID NO: 924.

243. The antibody or antigen-binding fragment thereof of claim 175, wherein the VH comprises the sequence of SEQ ID NO: 925 and the VL comprises the sequence of SEQ ID NO: 926.

244. The antibody or antigen-binding fragment thereof of claim 175, wherein the VH comprises the sequence of SEQ ID NO: 927 and the VL comprises the sequence of SEQ ID NO: 928.

245. The antibody or antigen-binding fragment thereof of claim 175, wherein the VH comprises the sequence of SEQ ID NO: 929 and the VL comprises the sequence of SEQ ID NO: 930.

246. The antibody or antigen-binding fragment thereof of claim 175, wherein the VH comprises the sequence of SEQ ID NO: 931 and the VL comprises the sequence of SEQ ID NO: 932.

247. The antibody or antigen-binding fragment thereof of claim 175, wherein the VH comprises the sequence of SEQ ID NO: 933 and the VL comprises the sequence of SEQ ID NO: 934.

248. The antibody or antigen-binding fragment thereof of claim 175, wherein the VH comprises the sequence of SEQ ID NO: 935 and the VL comprises the sequence of SEQ ID NO: 936.

249. The antibody or antigen-binding fragment thereof of claim 175, wherein the VH comprises the sequence of SEQ ID NO: 937 and the VL comprises the sequence of SEQ ID NO: 938.

250. The antibody or antigen-binding fragment thereof of claim 175, wherein the VH comprises the sequence of SEQ ID NO: 939 and the VL comprises the sequence of SEQ ID NO: 940.

251. The antibody or antigen-binding fragment thereof of claim 175, wherein the VH comprises the sequence of SEQ ID NO: 941 and the VL comprises the sequence of SEQ ID NO: 942.

252. The antibody or antigen-binding fragment thereof of claim 175, wherein the VH comprises the sequence of SEQ ID NO: 943 and the VL comprises the sequence of SEQ ID NO: 944.

253. The antibody or antigen-binding fragment thereof of claim 175, wherein the VH comprises the sequence of SEQ ID NO: 945 and the VL comprises the sequence of SEQ ID NO: 946.

254. The antibody or antigen-binding fragment thereof of claim 175, wherein the VH comprises the sequence of SEQ ID NO: 947 and the VL comprises the sequence of SEQ ID NO: 948.

255. The antibody or antigen-binding fragment thereof of claim 175, wherein the VH comprises the sequence of SEQ ID NO: 949 and the VL comprises the sequence of SEQ ID NO: 950.

256. The antibody or antigen-binding fragment thereof of claim 175, wherein the VH comprises the sequence of SEQ ID NO: 951 and the VL comprises the sequence of SEQ ID NO: 952.

257. The antibody or antigen-binding fragment thereof of claim 175, wherein the VH comprises the sequence of SEQ ID NO: 953 and the VL comprises the sequence of SEQ ID NO: 954.

258. The antibody or antigen-binding fragment thereof of claim 175, wherein the VH comprises the sequence of SEQ ID NO: 955 and the VL comprises the sequence of SEQ ID NO: 956.

259. The antibody or antigen-binding fragment thereof of claim 175, wherein the VH comprises the sequence of SEQ ID NO: 957 and the VL comprises the sequence of SEQ ID NO: 958.

260. The antibody or antigen-binding fragment thereof of claim 175, wherein the VH comprises the sequence of SEQ ID NO: 959 and the VL comprises the sequence of SEQ ID NO: 960.

261. The antibody or antigen-binding fragment thereof of claim 175, wherein the VH comprises the sequence of SEQ ID NO: 961 and the VL comprises the sequence of SEQ ID NO: 962.

262. The antibody or antigen-binding fragment thereof of claim 175, wherein the VH comprises the sequence of SEQ ID NO: 963 and the VL comprises the sequence of SEQ ID NO: 964.

263. The antibody or antigen-binding fragment thereof of claim 175, wherein the VH comprises the sequence of SEQ ID NO: 965 and the VL comprises the sequence of SEQ ID NO: 966.

264. The antibody or antigen-binding fragment thereof of claim 175, wherein the VH comprises the sequence of SEQ ID NO: 967 and the VL comprises the sequence of SEQ ID NO: 968.

265. The antibody or antigen-binding fragment thereof of claim 175, wherein the VH comprises the sequence of SEQ ID NO: 969 and the VL comprises the sequence of SEQ ID NO: 970.

266. The antibody or antigen-binding fragment thereof of claim 175, wherein the VH comprises the sequence of SEQ ID NO: 971 and the VL comprises the sequence of SEQ ID NO: 972.

267. The antibody or antigen-binding fragment thereof of claim 175, wherein the VH comprises the sequence of SEQ ID NO: 973 and the VL comprises the sequence of SEQ ID NO: 974.

268. The antibody or antigen-binding fragment thereof of claim 175, wherein the VH comprises the sequence of SEQ ID NO: 975 and the VL comprises the sequence of SEQ ID NO: 976.

269. The antibody or antigen-binding fragment thereof of claim 175, wherein the VH comprises the sequence of SEQ ID NO: 977 and the VL comprises the sequence of SEQ ID NO: 978.

270. The antibody or antigen-binding fragment thereof of claim 175, wherein the VH comprises the sequence of SEQ ID NO: 979 and the VL comprises the sequence of SEQ ID NO: 980, 981, 982, 983, or 984.

271. The antibody or antigen-binding fragment thereof of claim 175, wherein the VH comprises the sequence of SEQ ID NO: 985 and the VL comprises the sequence of SEQ ID NO: 986.

272. The antibody or antigen-binding fragment thereof of claim 175, wherein the VH comprises the sequence of SEQ ID NO: 987, 1687, or 1688, and the VL comprises the sequence of SEQ ID NO: 988, 989, 990, 991, or 992.

273. The antibody or antigen-binding fragment thereof of claim 175, wherein the VH comprises the sequence of SEQ ID NO: 993 and the VL comprises the sequence of SEQ ID NO: 994.

274. The antibody or antigen-binding fragment thereof of claim 175, wherein the VH comprises the sequence of SEQ ID NO: 995 and the VL comprises the sequence of SEQ ID NO: 996.

275. The antibody or antigen-binding fragment thereof of claim 175, wherein the VH comprises the sequence of SEQ ID NO: 997 and the VL comprises the sequence of SEQ ID NO: 998.

276. The antibody or antigen-binding fragment thereof of claim 175, wherein the VH comprises the sequence of SEQ ID NO: 999 and the VL comprises the sequence of SEQ ID NO: 1000, 1001, 1002, 1003, or 1004.

277. The antibody or antigen-binding fragment thereof of claim 175, wherein the VH comprises the sequence of SEQ ID NO: 1005 and the VL comprises the sequence of SEQ ID NO: 1006.

278. The antibody or antigen-binding fragment thereof of claim 175, wherein the VH comprises the sequence of SEQ ID NO: 1007 and the VL comprises the sequence of SEQ ID NO: 1008, 1009, 1010.

279. The antibody or antigen-binding fragment thereof of claim 175, wherein the VH comprises the sequence of SEQ ID NO: 1011 and the VL comprises the sequence of SEQ ID NO: 1012.

280. The antibody or antigen-binding fragment thereof of claim 175, wherein the VH comprises the sequence of SEQ ID NO: 1013 and the VL comprises the sequence of SEQ ID NO: 1014.

281. The antibody or antigen-binding fragment thereof of claim 175, wherein the VH comprises the sequence of SEQ ID NO: 1015 and the VL comprises the sequence of SEQ ID NO: 1016.

282. The antibody or antigen-binding fragment thereof of claim 175, wherein the VH comprises the sequence of SEQ ID NO: 1017 and the VL comprises the sequence of SEQ ID NO: 1018.

283. The antibody or antigen-binding fragment thereof of claim 175, wherein the VH comprises the sequence of SEQ ID NO: 1019 and the VL comprises the sequence of SEQ ID NO: 1020.

284. The antibody or antigen-binding fragment thereof of claim 175, wherein the VH comprises the sequence of SEQ ID NO: 1021 and the VL comprises the sequence of SEQ ID NO: 1022.

285. The antibody or antigen-binding fragment thereof of claim 175, wherein the VH comprises the sequence of SEQ ID NO: 1023 and the VL comprises the sequence of SEQ ID NO: 1024.

286. The antibody or antigen-binding fragment thereof of claim 175, wherein the VH comprises the sequence of SEQ ID NO: 1025 and the VL comprises the sequence of SEQ ID NO: 1026.

287. The antibody or antigen-binding fragment thereof of claim 175, wherein the VH comprises the sequence of SEQ ID NO: 1027 and the VL comprises the sequence of SEQ ID NO: 1028.

288. The antibody or antigen-binding fragment thereof of claim 175, wherein the VH comprises the sequence of SEQ ID NO: 1029 and the VL comprises the sequence of SEQ ID NO: 1030.

289. The antibody or antigen-binding fragment thereof of claim 175, wherein the VH comprises the sequence of SEQ ID NO: 1031 and the VL comprises the sequence of SEQ ID NO: 1032.

290. The antibody or antigen-binding fragment thereof of claim 175, wherein the VH comprises the sequence of SEQ ID NO: 1033 and the VL comprises the sequence of SEQ ID NO: 1034.

291. The antibody or antigen-binding fragment thereof of claim 175, wherein the VH comprises the sequence of SEQ ID NO: 1035 and the VL comprises the sequence of SEQ ID NO: 1036.

292. The antibody or antigen-binding fragment thereof of claim 175, wherein the VH comprises the sequence of SEQ ID NO: 1037 and the VL comprises the sequence of SEQ ID NO: 1038.

293. The antibody or antigen-binding fragment thereof of claim 175, wherein the VH comprises the sequence of SEQ ID NO: 1039 and the VL comprises the sequence of SEQ ID NO: 1040.

294. The antibody or antigen-binding fragment thereof of claim 175, wherein the VH comprises the sequence of SEQ ID NO: 1041 and the VL comprises the sequence of SEQ ID NO: 1042.

295. The antibody or antigen-binding fragment thereof of claim 175, wherein the VH comprises the sequence of SEQ ID NO: 1043 and the VL comprises the sequence of SEQ ID NO: 1044, 1689, or 1690.

296. The antibody or antigen-binding fragment thereof of claim 175, wherein the VH comprises the sequence of SEQ ID NO: 1045 and the VL comprises the sequence of SEQ ID NO: 1046.

297. The antibody or antigen-binding fragment thereof of claim 175, wherein the VH comprises the sequence of SEQ ID NO: 1047 and the VL comprises the sequence of SEQ ID NO: 1048.

298. The antibody or antigen-binding fragment thereof of claim 175, wherein the VH comprises the sequence of SEQ ID NO: 1049 and the VL comprises the sequence of SEQ ID NO: 1050.

299. The antibody or antigen-binding fragment thereof of claim 175, wherein the VH comprises the sequence of SEQ ID NO: 1051 and the VL comprises the sequence of SEQ ID NO: 1052.

300. The antibody or antigen-binding fragment thereof of claim 175, wherein the VH comprises the sequence of SEQ ID NO: 1053 and the VL comprises the sequence of SEQ ID NO: 1054.

301. The antibody or antigen-binding fragment thereof of claim 175, wherein the VH comprises the sequence of SEQ ID NO: 1055 and the VL comprises the sequence of SEQ ID NO: 1056.

302. The antibody or antigen-binding fragment thereof of any one of claims 175-301, wherein the antibody or antigen-binding fragment specifically binds to human LILRB1 and/or LILRB2.

303. The antibody or antigen-binding fragment thereof of any one of claims 175-302, wherein the antibody or antigen-binding fragment is a humanized antibody or antigen-binding fragment thereof.

304. The antibody or antigen-binding fragment thereof of any one of claims 175-303, wherein the antibody or antigen-binding fragment is a single-chain variable fragment (scFV) or a multi-specific antibody (e.g., a bispecific antibody).

305. An antibody-drug conjugate comprising the antibody or antigen-binding fragment thereof of any one of claims 1-58 and 175-304 covalently bound to a therapeutic agent.

306. The antibody drug conjugate of claim 305, wherein the therapeutic agent is a cytotoxic or cytostatic agent.

307. A method of treating a subject having cancer, the method comprising administering a therapeutically effective amount of a composition comprising the antibody or antigen-binding fragment thereof of any one of claims 1-58 and 175-304, or the antibody-drug conjugate of claims 227 or 228, to the subject.

308. The method of claim 307, wherein the subject has a solid tumor or hematological cancer.

309. The method of claim 307, wherein the cancer is glioma, thyroid cancer, lung cancer, colorectal cancer, head and neck cancer, stomach cancer, liver cancer, pancreatic cancer, renal cancer, urothelial cancer, prostate cancer, testis cancer, breast cancer, cervical cancer, endometrial cancer, ovarian cancer, or melanoma.

310. A method of decreasing the rate of tumor growth, the method comprising contacting a tumor cell with an effective amount of a composition comprising an antibody or antigen-binding fragment thereof of any one of claims 1-58 and 175-304, or the antibody-drug conjugate of claims 305 or 306.

311. A method of killing a tumor cell, the method comprising contacting a tumor cell with an effective amount of a composition comprising the antibody or antigen-binding fragment thereof of any one of claims 1-58 and 175-304, or the antibody-drug conjugate of claims 305 or 306.

312. A pharmaceutical composition comprising the antibody or antigen-binding fragment thereof of any one of claims 1-58 and 175-304, and a pharmaceutically acceptable carrier.

313. A pharmaceutical composition comprising the antibody drug conjugate of claim 305 or 306, and a pharmaceutically acceptable carrier.
